(19) United States
(12) Patent Application Publication
(10) Pub. No.: US 2024/0300951 A1
(43) Pub. Date: Sep. 12, 2024

(54) Pyrazolopyridinone Compounds

(71) Applicants: BeiGene Ltd., Grand Cayman (KY); Beigene Switzerland GMBH, Basel (CH)

(72) Inventors: Guoliang Zhang, Beijing (CN); Zhikun Ni, Beijing (CN); Jianzhuang Miao, Beijing (CN); Ce Wang, Beijing (CN)

(73) Assignees: BeiGene Ltd., Grand Cayman (KY); Beigene Switzerland GMBH, Basel (CH)

(21) Appl. No.: 18/431,626

(22) Filed: Feb. 2, 2024

Related U.S. Application Data
(63) Continuation of application No. PCT/CN2022/109663, filed on Aug. 2, 2022.

(30) Foreign Application Priority Data
Aug. 3, 2021 (WO) PCT/CN2021/110407
Jul. 5, 2022 (WO) PCT/CN2022/103862
Jul. 19, 2022 (WO) PCT/CN2022/106484

(51) Int. Cl. C07D 487/04; A61K 31/437; A61K 31/444; A61K 31/496; A61K 31/498; A61K 31/501; A61K 31/519; A61K 31/5517; C07D 471/04

(52) U.S. Cl. CPC C07D 487/04; A61K 31/437; A61K 31/444; A61K 31/496; A61K 31/498; A61K 31/501; A61K 31/519; A61K 31/5517; C07D 471/04

(57) ABSTRACT

Disclosed herein is a compound of Formula (I) for activating T cells, promoting T cell proliferation, and/or exhibiting antitumor activity, a method of using the compounds disclosed herein for treating cancer, and a pharmaceutical composition comprising the same.

### FIELD OF THE DISCLOSURE

Disclosed herein is a compound of Formula (I) for activating T cells, promoting T cell proliferation, and/or exhibiting antitumor activity, a method of using the compounds disclosed herein for treating cancer, and a pharmaceutical composition comprising the same.

### BACKGROUND OF THE DISCLOSURE

Diacylglycerol kinases (DGKs) are a family of lipid kinases that phosphorylates and converts diacylglycerol (DAG) into phosphatidic acid (PA). As the substrate of DGKs, DAG is generated from inositol phospholipids and other phospholipids at the plasma membrane by phospholipase C (PLC) hydrolysis in response to the activation of various cell-surface receptors, including G-protein coupled receptors (GPCR) and immunoreceptor tyrosine-based activation motif (ITAM)-bearing receptors (Rhee, Sue Goo. Annual review of biochemistry. 2001, 70.1: 281-312). DAG is one of the key intracellular second messengers that recruits and activates many downstream effectors including protein kinase C (PKC), protein kinase D (PKD) families, and Ras guanyl nucleotide releasing proteins (RasGRPs), which in turn activates NF-κB and extracellular regulated kinase (ERK) pathways (Mérida, Isabel, et al. Biochemical Journal. 2008, 409.1: 1-18, Joshi, Rohan P., et al. International Journal of Molecular Sciences. 2013, 14.4: 6649-6673). By consuming DAG, DGK controls and tunes the threshold and duration of DAG mediated signaling. Mammalian DGK family comprises 10 different members, in which DGKα, DGKζ and DGKδ are the three major isoforms that abundantly expressed in lymphoid tissues (Joshi, Rohan P., et al. International Journal of Molecular Sciences. 2013, 14.4: 6649-6673).

Cancer immunotherapy is a type of cancer treatment to manipulate and boost host immune system to recognize and attack cancer cells. A vast majority of studies have focused on targeting immune checkpoint inhibitors, such as CTLA-4 and PD-1/PD-L1, to reinvigorate exhausted CD8⁺ T cells within tumor sites. It was emerged that peripheral T cell tolerance, which under normal circumstances prevents detrimental autoimmune disease, can be hijacked by tumors to prevent anti-tumor immune response during carcinogenesis (Ntissing, Simone, et al. Frontiers in Immunology. 2020, 11: 2461). T cell anergy is a one of the most important mechanisms of T cell tolerance and has been reported to occur in tumor infiltrated T cells, which contributes to the immunosuppressive nature of tumor microenvironment (Abe, Brian T., and Fernando Macian. Oncoimmunology. 2013, 2.2: e22679). Anergy-associated transcription factor early growth response gene2 (Egr2) directly binds to Dgka and Dgkz promoter and increases their expression (Zheng, Yan, et al. Journal of Experimental Medicine 2012, 209.12: 2157-2163; Zheng, Yan, et al. Molecular Immunology. 2013, 55.3-4: 283-291). In anergic T cells, both DGKα and DGKζ play critical roles to negatively regulate DAG-signaling downstream of TCR and reduce the strength of TCR activation (Chen, Shelley S., et al. Frontiers in Cell and Developmental Biology. 2016, 4: 130). Thus, immune cell expressed DGKα and DGKζ were investigated as potential targets to reverse the hyporesponsiveness of the tumor infiltrated T cells. It was demonstrated that genetic deletion of DGKα or DGKζ enhanced cytokine production and proliferation of T cells (Olenchock, Benjamin A., et al. Nature immunology. 2006, 7.11: 1174-1181; Zhong, Xiao-Ping, et al. Nature immunology. 2003, 4.9: 882-890). DGKα or DGKζ single knockout in both mouse or human chimeric antigen receptor (CAR)-T cells showed superior effector function as determined by enhanced in vitro cytotoxicity and cytokine secretion when cocultured with antigen expressing titled cells (Riese, Matthew J., et al. Cancer Research. 2013, 73.12: 3566-3577; Jung, In-Young, et al. Cancer Research. 2018, 78.16: 4692-4703). MesoCAR-transduced DGKα or DGKζ deficient T cells also showed significantly elevated in vivo activity against mesotheliomas (Riese, Matthew J., et al. Cancer Research. 2013, 73.12: 3566-3577). DGKζ^{-/-} mice showed enhanced tumor suppressive efficacy with both orthotopic and subcutaneously implanted models (Wesley, Erin M., et al. Immunohorizons. 2018, 2.4: 107-118; Wee, Susan, et al. AACR; Cancer Res 2019; 79 (13 Suppl):Abstract nr 936). Besides the T cell regulatory function, both DGKα and DGKζ also involve in tuning NK cell activation at tumor site (Prinz, Petra U., et al. International Journal of Cancer. 2014. 135.8: 1832-1841; Yang, Enjun, et al. The Journal of Immunology. 2016, 197.3: 934-941). In addition, DGKζ were found to play a critical role to control the activation threshold of mature B cells (Wheeler, Matthew L., et al. Science Signaling, 2013, 6.297: ra91-ra91). In summary, all these preclinical data suggest titled inhibition of DGKα and DGKζ could be therapeutic beneficial to promote immunity against cancer.

Although the existing anti-CTLA-4 and anti-PD-1 therapies have shown clear clinical benefits in a subset of patients with various tumor types, there are still unmet medical needs to develop novel immunotherapies to achieve robust and durable clinical anti-tumor efficacy. Pre-clinical data strongly suggests there is great potential of developing DGKα and DGKζ targeted therapies to improve antitumor immunity.

### SUMMARY OF THE DISCLOSURE

The above needs have been met by providing the compounds disclosed herein which have a novel core structure and show the desired inhibition of DGKα and DGKζ. In some embodiments, the compounds disclosed herein show the dual inhibitory activity of both DGKα and DGKζ. In some embodiments, the compounds disclosed herein show the selective inhibitory activity of DGKα over DGKζ. In some embodiments, the compounds disclosed herein show the selective inhibitory activity of DGKζ over DGKα.

Disclosed herein provides a compound of formula (I),

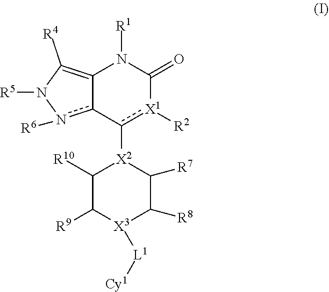

or a stereoisomer or a pharmaceutically acceptable salt thereof
wherein
X¹ is C or N,
each of X² and X³ is independently selected form —N— or —CH—;
the symbol [symbol] is a single or double bond,
R¹ is hydrogen, or alkyl optionally substituted with deuterium, halogen, hydroxy, alkoxy or cycloalkyl;
R² is hydrogen, halogen, alkyl or cyano, provided that R² is absent when X¹ is N and the bond [symbol] attached to X¹ is a double bond;
R⁴ is hydrogen, halogen or alkyl, wherein the alkyl is optionally substituted with halogen or —OR^{4a}, wherein R^{4a} is hydrogen, alkyl, alkenyl, alkynyl, cycloalkyl or heterocycloalkyl, wherein alkyl, alkenyl, alkynyl, cycloalkyl or heterocycloalkyl is optionally substituted with —C₁₋₆alkyl, —C₁₋₆alkoxy or —C₃₋₈ cycloalkyl;
R⁵ is hydrogen, alkyl, alkenyl, alkynyl, cycloalkyl, R^{5a}—C(O)—, R^{5a}—C(O)O—, R^{5a}—O—C(O)—, R^{5a}—C(O)NR^{5b}—, R^{5a}—NR^{5b}—C(O)—, R^{5a}—SO₂— or heterocyclyl, wherein said alkyl or alkenyl is unsubstituted or substituted with halogen, cyano, —C(O)OR^{5c}, —C(O)R^{5e}, —C(O)NR^{5c}R^{5d}, heterocyclyl, alkoxy, hydroxy, cycloalkyl, or NR^{5c}R^{5d}; and wherein each of said cycloalkyl and heterocyclyl is unsubstituted or substituted with alkyl, cyano or halogen substituted alkyl, cyano, —C(O)OR^{5c}, —C(O)R^{5c}, —C(O)NR^{5c}R^{5d}, heterocyclyl, alkoxy, hydroxy, cycloalkyl, NR^{5c}R^{5d}, or R^{5c}—SO₂—, wherein R^{5a} and R^{5b} are each independently hydrogen, alkyl, or cycloalkyl; and wherein R^{5c} and R^{5d} are hydrogen or alkyl;
R⁶ is hydrogen, halogen, alkyl which is unsubstituted or substituted with halogen or cyano, provided that R⁶ is absent when the bond [symbol] attached to the nitrogen to which R⁶ is attached is a double bond; each of R⁷, R⁹, R⁸, and R¹⁰ is independently hydrogen, alkyl, alkoxy, or —C(O)R^{7a}, wherein said alkyl is unsubstituted or substituted with halogen, cyano, hydroxy, or alkoxy, and wherein R^{7a} is hydrogen, alkyl, or alkoxy, provided that at least one of R⁷ and R⁹ is not hydrogen;
or R⁷ and R⁹ are each hydrogen and R⁸ and R¹⁰ together form a bridge containing at least one —CH₂-moiety in addition to the two bridgehead atoms; or R⁸ and R¹⁰ are each hydrogen and R⁷ and R⁹ together form a bridge containing at least one —CH₂— moiety in addition to the two bridgehead atoms;
L¹ is a direct bond, —O—, —N(R^{L})—, -alkylene- or —C(O)—, wherein said -alkylene- is unsubstituted or substituted with —C(O)NR^{L1}, and R^{L} is hydrogen or alkyl, wherein R^{L1} is hydrogen, alkyl, or alkoxyalkyl;
Cy¹ is aryl, heterocyclyl, heteroaryl, or cycloalkyl, each of which is unsubstituted or substituted with one, two or three substituents R^{3a}, wherein R^{3a} is selected from deuterium, hydroxy, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, cycloalkyl, heterocyclyl or heterocyclyloxy, wherein said alkyl moiety in the group alkyl or alkoxy is unsubstituted or substituted with deuterium, halogen, alkoxy, hydroxy, cyano, or heterocyclyl; said cycloalkyl or heterocyclyl is unsubstituted or substituted with alkoxy, alkyl, halogen, or hydroxy, wherein R^{3b} and R^{3c} are each independently hydrogen or alkyl.

In some embodiments, the compound of formula (I) is anyone of the following subgenus:

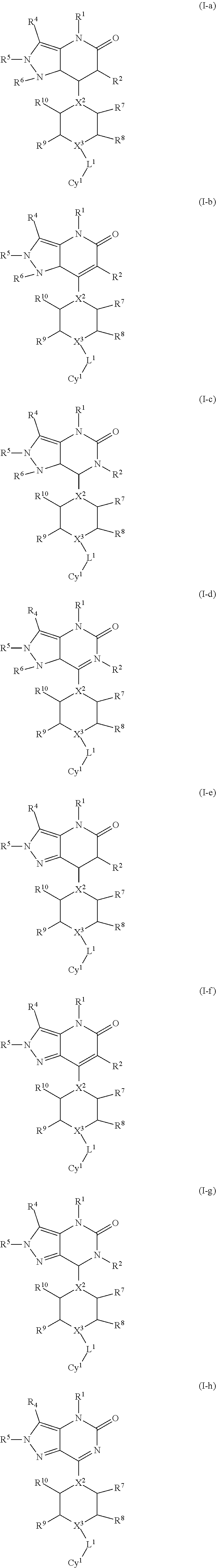

wherein the variables are defined as herein.

### The Definitions of R¹

In some embodiments, R¹ is hydrogen, or alkyl optionally substituted with deuterium, halogen, hydroxy, alkoxy or cycloalkyl. In some embodiments, R¹ is hydrogen, or C₁₋₄alkyl optionally substituted with deuterium, halogen, hydroxy, alkoxy or cycloalkyl. In some embodiments, R¹ is hydrogen, or C₁₋₃alkyl optionally substituted with deuterium, or halogen. In some embodiments, R¹ is hydrogen, or C₁₋₃alkyl optionally substituted with deuterium.

In some embodiments, R¹ is hydrogen, methyl, methyl-d3, ethyl, isopropyl, 2-hydroxyethyl, 2-methoxyethyl, 2,2,2-trifluoroethyl, 2,2-difluoroethyl, or cyclopropylmethyl. In some embodiments, R¹ is hydrogen, methyl, ethyl or methyl-d3. In some embodiments, R¹ is methyl or methyl-d3. In some embodiments, R¹ is methyl.

### The Definitions of R²

In some embodiments, R² is hydrogen, halogen, alkyl or cyano, provided that R² is absent when X¹ is N and the bond [symbol] attached to X¹ is a double bond. In some embodiments, R² is hydrogen, halogen, C₁₋₄alkyl or cyano. In some embodiments, R² is hydrogen, F, Br, Cl or CN.

### The Definitions of R⁴

In some embodiments, R⁴ is hydrogen, halogen or alkyl, wherein the alkyl is optionally substituted with halogen or —OR^{4a}, wherein R^{4a} is hydrogen, alkyl, alkenyl, alkynyl, cycloalkyl or heterocycloalkyl, wherein alkyl, alkenyl, alkynyl, cycloalkyl or heterocycloalkyl is optionally substituted with —C₁₋₆alkyl, —C₁₋₆alkoxy or —C₃₋₈cycloalkyl. In some embodiments, R⁴ is hydrogen, halogen or C₁₋₄alkyl, wherein the alkyl is optionally substituted with halogen or —OR^{4a}. In some embodiments, R⁴ is hydrogen, halogen or C₁₋₄alkyl, wherein the alkyl is optionally substituted with halogen.

In some embodiments, R⁴ is hydrogen, fluoro, chloro, bromo, methyl, trifluoromethyl, ethyl, or 2,2,2-trifluoroethyl. In some embodiments, R⁴ is hydrogen.

### The Definitions of R⁵

In some embodiments, R⁵ is hydrogen, alkyl, alkenyl, alkynyl, cycloalkyl, R^{5a}—C(O)—, R^{5a}—C(O)O—, R^{5a}—O—C(O)—, R^{5a}—C(O)NR^{5b}, R^{5a}—NR^{5b}—C(O)—, R^{5a}—SO₂— or heterocyclyl, wherein said alkyl or alkenyl is unsubstituted or substituted with halogen, cyano, —C(O)OR^{5b}, —C(O)R^{5c}, —C(O)NR^{5c}R^{5d}, heterocyclyl, alkoxy, hydroxy, cycloalkyl, or NR^{5c}R^{5d}; and wherein each of said cycloalkyl and heterocyclyl is unsubstituted or substituted with alkyl, cyano or halogen substituted alkyl, cyano, —C(O)OR^{5c}, —C(O)R^{5c}, —C(O)NR^{5c}R^{5d}, heterocyclyl, alkoxy, hydroxy, cycloalkyl, NR^{5c}R^{5d}, or R^{5c}—SO₂—, wherein R^{5a} and R^{5b} are each independently hydrogen, alkyl, or cycloalkyl; and wherein R^{5c} and R^{5d} are hydrogen or alkyl.

In some embodiments, R⁵ is hydrogen, alkyl, alkenyl or alkynyl, wherein said alkyl is unsubstituted or substituted with cyano. In some embodiments, R⁵ is C₁₋₄alkyl, C₂₋₄alkenyl or C₂₋₄alkynyl, wherein said alkyl is substituted with cyano.

In some embodiments, R⁵ is hydrogen, CN—CH₂—, —CH₂C(O)—OMe, —CH(CH₃)CN, oxiran-2-ylmethyl-, prop-2-yn-1-yl, but-2-yn-1-yl, prop-1-en-2-yl, but-2-en-1-yl, but-3-en-1-yl, methyl, isopropyl, —CH₂CH₂—O—Me, —CH₂C(O)NH₂, —CH₂CH₂—OH, cyclopropyl-CH₂—, —CH₂CH₂N(CH₃)₂, CH₃—SO₂—, cyclopropyl, cyclobutyl, cyclopropyl-C(O)—, 1-cyanocyclopropyl, 2-cyanocyclopropyl, 2-cyanocyclobutyl, 3-(cyanomethyl)-1-(ethylsulfonyl)azetidine-3-yl, or 1-cyano-2-cyclopentyleth-2-yl. Preferably, R⁵ is hydrogen, CN—CH₂—, —CH₂C(O)—OMe, —CH(CH₃)CN, oxiran-2-ylmethyl-, prop-2-yn-1-yl, but-2-yn-1-yl, or prop-1-en-2-yl. More preferably, R⁵ is CN—CH₂—, prop-2-yn-1-yl, but-2-yn-1-yl, or prop-1-en-2-yl.

In some embodiments, R⁵ is CN—CH₂—, —CH(CH₃)CN, prop-2-yn-1-yl, but-2-yn-1-yl, or prop-1-en-2-yl.

In some embodiments, R⁵ is CN—CH₂—. In some embodiments, R⁵ is —CH(CH₃)CN. In some embodiments, R⁵ is prop-2-yn-1-yl. In some embodiments, R⁵ is but-2-yn-1-yl. In some embodiments, R⁵ is prop-1-en-2-yl.

### The Definitions of R⁶

In some embedment, R⁶ is absent, hydrogen, halogen, alkyl which is unsubstituted or substituted with halogen or cyano, provided that R⁶ is absent when the bond [symbol] attached to the nitrogen to which R⁶ is attached is a double bond. In some embodiments, R⁶ is absent. In some embodiments, R⁶ is hydrogen, F, Br, Cl or C₁₋₄alkyl which is unsubstituted or substituted with cyano.

### The Definitions of R⁷/R⁹, R⁸/R¹⁰

In some embodiments, each of R⁷, R⁹, R⁸, and R¹⁰ is independently hydrogen, alkyl, or —C(O)R^{7a}, wherein said alkyl is unsubstituted or substituted with halogen, cyano, hydroxy, or alkoxy, and wherein R^{7a} is hydrogen, alkyl, or alkoxy, provided that at least one of R⁷ and R⁹ is not hydrogen.

In some embodiments, each of R⁷ and R⁹ is independently hydrogen, alkyl, or —C(O)R^{7a}, wherein said alkyl is unsubstituted or substituted with halogen, cyano, hydroxy, or alkoxy, and wherein R^{7a} is hydrogen, alkyl, or alkoxy. In some embodiments, each of R⁷ and R⁹ is independently C₁₋₄alkyl. In some embodiments, each of R⁷ and R⁹ is independently C₁₋₂alkyl.

In some embodiments, R⁷ and R⁹ are each independently hydrogen, methyl, ethyl, methoxymethyl, 1-hydroxyethyl, 2-methoxyethyl, cyanomethyl, hydroxyethyl, hydroxymethyl, methoxycarbonyl, difluoromethyl, provided that at least one of R⁷ and R⁹ is not hydrogen.

In some embodiments, R⁸ and R¹⁰ are each hydrogen.

In some embodiments, R⁷ is methyl, and R⁹ is methyl; or R⁷ is ethyl, and R⁹ is ethyl; or R⁷ is methyl, and R⁹ is ethyl; or R⁷ is methyl, and R⁹ is methoxycarbonyl; or R⁷ is hydrogen, and R⁹ is methyl; or R⁷ is hydrogen, and R⁹ is ethyl.

In some embodiments, R⁷ and R⁹ are each hydrogen and R⁸ and R¹⁰ together form a bridge containing at least one —CH₂— moiety in addition to the two bridgehead atoms. In some embodiments, R⁷ and R⁹ are each hydrogen and R⁸ and R¹⁰ together form a bridge containing one —CH₂— moiety in addition to the two bridgehead atoms. In some embodiments, R⁷ and R⁹ are each hydrogen and R⁸ and R¹⁰ together form a bridge containing two —CH₂— moieties in addition to the two bridgehead atoms.

In some embodiments, R⁸ and R¹⁰ are each hydrogen and R⁷ and R⁹ together form a bridge containing at least one —CH₂— moiety in addition to the two bridgehead atoms. In some embodiments, R⁸ and R¹⁰ are each hydrogen and R⁷ and R⁹ together form a bridge containing one —CH₂— moiety in addition to the two bridgehead atoms. In some embodiments, R⁸ and R¹⁰ are each hydrogen and R⁷ and R⁹ together form a bridge containing two —CH₂— moieties in addition to the two bridgehead atoms.

### The Definitions of L¹

In some embodiments, L¹ is a direct bond, —O—, —N(R^{L})—, -alkylene- or —C(O)—, wherein R^{L} is hydrogen or alkyl. In some embodiments, L¹ is C₁₋₄alkylene, preferably C₁₋₂alkylene. In some embodiments, L¹ is a direct bond, —CH₂—, —CH(CH₃)—, —CH(CH₂CH₃)—, —CH(CHF₂)—, —N(H)—, —N(CH₃)—, —O—, —CH(C(O)—NHCH₂CH₂OCH₃)— or —C(CH₃)₂—. In some embodiments, L¹ is —CH₂— or —CH(CH₃)—.

### The Definition of X² and X³

In some embodiments, X² and X³ are N or CH. In some embodiments, X² is N, and X³ is N; or X² is N, and X³ is CH; or X² is CH, and X³ is N; or X² is CH, and X³ is N.

### The Definitions of Cy¹

In some embodiments, Cy¹ is aryl, heterocyclyl, heteroaryl, or cycloalkyl, each of which is unsubstituted or substituted with one, two or three substituents R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, heterocyclyl, or heterocyclyloxy, wherein said alkyl moiety in the group alkyl or alkoxy is unsubstituted or substituted with deuterium, halogen, alkoxy, hydroxy, cyano or heterocyclyl; said cycloalkyl or heterocyclyl is unsubstituted or substituted with alkoxy, alkyl, halogen, or hydroxy, wherein R^{3b} and R^{3c} are each independently hydrogen or alkyl.

In some embodiments, Cy¹ is aryl, which is unsubstituted or substituted with one, two or three substituents R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, heterocyclyl, or heterocyclyloxy wherein said alkyl moiety in the group alkyl or alkoxy is unsubstituted or substituted with deuterium, halogen, alkoxy, hydroxy, cyano or heterocyclyl; said cycloalkyl or heterocyclyl is unsubstituted or substituted with alkoxy, alkyl, halogen, or hydroxy, wherein R^{3b} and R^{3c} are each independently hydrogen or alkyl.

In some embodiments, Cy¹ is aryl, which is unsubstituted or substituted with one, two or three substituents R^{3a}, wherein R^{3a} is selected from alkoxy, halogen-substituted alkoxy, alkoxyalkyl-, alkyl, halogen-substituted alkyl, deuterium-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, heterocyclyl, or heterocyclyl-substituted alkyl, said cycloalkyl, heterocyclyl or heterocyclyloxy is unsubstituted or substituted with alkoxy, alkyl, halogen, or hydroxy, wherein R^{3b} and R^{3c} are each independently hydrogen or alkyl.

In some embodiments, Cy¹ is aryl, which is unsubstituted or substituted with one, two or three substituents R^{3a}, wherein R^{3a} is selected from deuterium, fluoro, bromo, chloro, methyl, methyl-d3, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, (oxetan-3-yl)methoxy, oxetan-3-yloxy, (tetrahydrofuran-3-yl)methoxy, (tetrahydro-2H-pyran-4-yl)oxy, 1-methylazetidin-3-yl, or 1-hydroxyazetidin-3-yl.

In some embodiments, Cy¹ is phenyl. In some embodiments, Cy¹ is phenyl, which is substituted with one R^{3a} as disclosed herein at position 4 and optionally substituted with R^{3a} on the other position.

In some embodiments, Cy¹ is naphthalenyl. In some embodiments, Cy¹ is naphthalen-1-yl, naphthalen-2-yl, naphthalen-3-yl, naphthalen-4-yl.

In some embodiments, Cy¹ is 2,3-dihydrobenzo[b][1,4]dioxin-6-yl, 3,3-dimethyl-2,3-dihydrobenzo[b][1,4]dioxin-6-yl, 2,2-difluorobenzo[d][1,3]dioxol-4-yl, 3,4-dihydro-2H-benzo[b][1,4]oxazin-6-yl, or

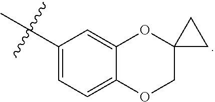 [SMILES: *c1ccc2c(c1)OC1(CC1)CO2]

In some embodiments, Cy¹ is a monocyclic 5- to 9-membered heterocyclyl or a bicyclic 7- to 10-membered heterocyclyl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with deuterium, halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl. In some embodiments, said monocyclic 5- to 9-membered heterocyclyl is tetrahydrofuranyl, tetrahydropyranyl, 1,4-dioxanyl, piperidinyl, piperazinyl, or dihydropyridinyl, each of which is unsubstituted or substituted with one, two or three R^{3a} as disclosed herein.

In some embodiments, Cy¹ is tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-1-yl, piperidin-2-yl, piperidin-3-yl, piperidin-4-yl, piperazin-1-yl, piperazin-2-yl, piperazin-3-yl, piperazin-4-yl, 1,2-dihydropyridin-3-yl, 1,2-dihydropyridin-4-yl, 1,2-dihydropyridin-5-yl, or 1,2-dihydropyridin-6-yl.

In some embodiments, Cy¹ is piperidinyl (e.g., piperidin-1-yl) or piperazinyl (e.g., piperazin-4-yl), which is substituted with one R^{3a} as disclosed herein at position 4 and optionally substituted with R^{3a} on the other position.

In some embodiments, said bicyclic 7- to 10-membered heterocyclyl is chromanyl, preferably chroman-2-yl, chroman-3-yl, chroman-4-yl, or chroman-6-yl; 2,3-dihydrofuro[2,3-b]pyridin-6-yl; 5,6,7,8-tetrahydroquinoxalin-2-yl; or benzo[d][1,3]dioxol-5-yl.

In some embodiments, Cy¹ is a monocyclic 5- to 9-membered heteroaryl or a bicyclic 7- to 10-membered heteroaryl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with deuterium, halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, methyl-d3, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, said monocyclic 5- to 9-membered heteroaryl is pyrazolyl, imidazolyl, pyridinyl, pyrimidinyl, pyrazinyl or pyridazinyl, each of which is unsubstituted or substituted with one, two or three R^{3a} as disclosed herein. In some embodiments, said monocyclic 5- to 9-membered heteroaryl is 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, pyrazin-2-yl, or pyridazin-4-yl, each of which is unsubstituted or substituted with one, two or three R^{3a} as disclosed herein.

In some embodiments, said bicyclic 7- to 10-membered heteroaryl is indolyl, benzo[d]imidazolyl, triazolopyridinyl, imidazopyridinyl, benzooxazolyl, benzo[d]thiazolyl, quinolinyl, isoquinolinyl, naphthyridinyl, dioxinopyridineyl, quinoxalinyl, benzo[d]imidazolyl, or imidazo[4,5-b]pyridinyl, each of which is unsubstituted or substituted with one, two or three R^{3a} as disclosed herein. In some embodiments, said bicyclic 7- to 10-membered heteroaryl is 1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, quinoxalin-6-yl-2,3-d2, 1H-indol-2-yl, 1H-benzo[d]imidazol-2-yl, 1-methyl-1H-benzo[d]imidazol-6-yl, 3H-imidazo[4,5-b]pyridin-2-yl, 4,5,6,7-tetrahydro-1H-benzo[d]imidazol-2-yl, 2,3-dihydro-[1,4]dioxino[2,3-b]pyridin-6-yl, or pyrazolo[1,5-a]pyridin-2-yl, each of which is unsubstituted or substituted with one, two or three R^{3a} as disclosed herein.

In some embodiments, Cy¹ is quinoxalinyl, e.g., quinoxalin-2-yl, quinoxalin-3-yl, quinoxalin-4-yl, quinoxalin-5-yl, quinoxalin-6-yl, quinoxalin-7-yl, quinoxalin-8-yl, preferably quinoxalin-6-yl, which is which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with deuterium, halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, methyl-d3, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, 1-hydroxyazetidin-3-yl, (2,2-dimethylmorpholino)methyl, 4-((2S,6R)-2,6-dimethylmorpholino)methyl, or (4,4-difluoropiperidin-1-yl)methyl.

In some embodiments, Cy¹ is quinoxalin-6-yl, which is which is unsubstituted or substituted with one, two or three R^{3a}, wherein R^{3a} is deuterium, methyl, ethyl, trifluoromethyl, methoxy, isopropoxy, difluoromethoxy, fluoro, chloro, cyano, amino, or cyclopropyl.

In some embodiments, Cy¹ is
a) phenyl, 2-(trifluoromethoxy)phenyl, 2-methoxyphenyl, 2-(methoxymethyl)phenyl, 2-(trifluoromethyl)phenyl, 4-fluoro-2-(methoxymethyl)phenyl, 4-fluoro-2-methoxyphenyl, 4-fluoro-2-methylphenyl, 2-bromo-4-fluorophenyl, 4-fluoro-2-(methylsulfonyl)phenyl, 4-methyl-2-(trifluoromethyl)phenyl, 2-chloro-4-fluorophenyl, 2,4-difluorophenyl, 2-ethoxy-4-fluorophenyl, 4-fluoro-2-isopropoxyphenyl, 4-fluoro-2-(trifluoromethoxy)phenyl, 2-(difluoromethoxy)-4-fluorophenyl, 2-(difluoromethyl)-4-fluorophenyl, 2-cyclopropyl-4-fluorophenyl, 4-fluoro-2-(1-hydroxyethyl)phenyl, 4-cyclopropyl-2-methoxyphenyl, 2-ethyl-4-fluorophenyl, 4-fluoro-2-(trifluoromethyl)phenyl, 2-methoxy-4-fluorophenyl, 2-(1,1-difluoroethyl)-4-fluorophenyl, 2-cyano-4-fluorophenyl, 4-fluoro-3-(methoxymethyl)phenyl, 3-methyl-2-(trifluoromethyl)phenyl, 4-fluoro-2,6-dimethoxyphenyl, 2,4-difluoro-6-methoxyphenyl, 2,6-dichloro-4-fluorophenyl, 4-cyclopropylphenyl, 4-methoxyphenyl, 4-fluorophenyl, 4-cyclopropyl-phenyl, 4-(trifluoromethyl)phenyl, 4-methylphenyl, 4-(difluoromethyl)phenyl, 4-isopropoxyphenyl, 2-fluoro-4-(trifluoromethyl)phenyl, 4-cyclopropyl-2-fluorophenyl, 4-fluoro-2-(trifluoromethyl)phenyl, 3-methoxy-4-(trifluoromethyl)phenyl, 4-fluoro-3-methoxyphenyl, 2,6-difluorophenyl, 4-(trifluoromethoxy)phenyl, 4-acetamidophenyl, 4-fluoro-2-(1-methoxyethyl)phenyl, 2-(cyanomethyl)-4-fluorophenyl, 3,4-difluoro-2-(trifluoromethyl)phenyl, 2-carbamoyl-4-fluorophenyl, 2-methoxycarbonyl-4-fluorophenyl, 2-(dimethylcarbamoyl)-4-fluorophenyl, 2-((difluoromethoxy)methyl)-4-fluorophenyl, 2-(1-(difluoromethoxy)ethyl)-4-fluorophenyl, 2-(azetidin-1-yl)-4-fluorophenyl, 4-fluoro-2-(2-methoxypropan-2-yl)phenyl, 4-(trifluoromethyl)phenyl, 3-(trifluoromethyl)phenyl, 4-fluoro-2-(1-methoxycyclopropyl)phenyl, 4-fluoro-2-(oxetan-2-yl)phenyl, 4-fluoro-2-(1-methylazetidin-3-yl)phenyl, 4-fluoro-2-(1-hydroxyazetidin-3-yl)phenyl, 4-cyclopropyl-2-methoxyphenyl; 2-ethyl-4-fluorophenyl; 4-fluoro-2-(trifluoromethyl)phenyl; 2-methoxy-4-fluorophenyl; 2,6-difluoro-4-methoxyphenyl; 2,5-difluoro-4-methoxyphenyl; 3-methoxy-4-(trifluoromethyl)phenyl; naphthalen-2-yl; 2-fluoro-4-methylphenyl; 4-((2,2-dimethylmorpholino)methyl)phenyl; 4-(((2S,6R)-2,6-dimethylmorpholino)methyl)phenyl; or 4-((4,4-difluoropiperidin-1-yl)methyl)phenyl; or
b) 2,3-dihydrobenzo[b][1,4]dioxin-6-yl, 3,3-dimethyl-2,3-dihydrobenzo[b][1,4]dioxin-6-yl, 2,2-difluorobenzo[d][1,3]dioxol-4-yl, 3,4-dihydro-2H-benzo[b][1,4]oxazin-6-yl, or

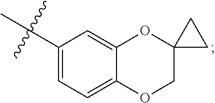 [SMILES: *c1ccc2c(c1)OC1(CC1)CO2]

or
c) tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-2-yl, piperidin-3-yl, piperidin-4-yl, piperazin-2-yl, piperazin-3-yl, 1,2-dihydropyridin-3-yl, 1,2-dihydropyridin-4-yl, 1,2-dihydropyridin-5-yl, 1,2-dihydropyridin-6-yl, 2,3-dihydrofuro[2,3-b]pyridin-6-yl, 2,3-dihydrofuro[3,2-b]pyridin-5-yl, 3,3-dimethyl-2,3-dihydrofuro[3,2-b]pyridin-5-yl, 5,6,7,8-tetrahydroquinoxalin-2-yl, 2,2-dimethylbenzo[d][1,3]dioxol-5-yl, or 2,2-difluorobenzo[d][1,3]dioxol-5-yl; or
d) chroman-2-yl, chroman-3-yl, chroman-4-yl, chroman-6-yl; or
e) 1H-pyrazol-3-yl, 1H-pyrazol-4-yl, 1H-pyrazol-5-yl, 1-ethyl-5-(trifluoromethyl)-1H-pyrazol-4-yl; 5-ethyl-1-methyl-1H-pyrazol-4-yl; 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, 3-methoxypyridin-2-yl, 3-fluoro-5-(trifluoromethyl)pyridin2-yl, 3-fluoro-5-methylpyridin-2-yl, 5-chloropyridin-2-yl, (5-(trifluoromethoxy)pyridin-2-yl, 3-fluoro-5-(trifluoromethoxy)pyridin-2-yl, 5,6-dimethylpyridin-2-yl, 6-methoxy-5-methylpyridin-2-yl, 4-fluoro-6-isopropoxypyridin-3-yl, 6-isopropoxypyridin-3-yl, 2-methoxypyridin-3-yl, 2-(trifluoromethyl)pyridin-3-yl, 6-(difluoromethoxy)pyridin-3-yl, 6-methylpyridin-3-yl, 6-cyclopropylpyridin-3-yl, 6-(difluoromethyl)pyridin-3-yl, 6-fluoropyridin-3-yl, 3-methoxypyridin-4-yl, 3-(trifluoromethyl)pyridin-4-yl, 5-fluoro-3-(trifluoromethyl)pyridin-2-yl, 5-chloro-1-ethyl-1H-imidazol-2-yl, 1-ethyl-2-(trifluoromethyl)-1H-imidazol-5-yl, -ethyl-5-(trifluoromethyl)-1H-pyrazol-4-yl, -ethyl-1-methyl-1H-pyrazol-4-yl, 1-methyl-5-(trifluoromethyl)-1H-pyrazol-4-yl, 5-(difluoromethyl)-1-methyl-1H-pyrazol-4-yl, 1-ethyl-3-(trifluoromethyl)-1H-pyrazol-5-yl, 3-chloro-1-ethyl-1H-pyrazol-5-yl, 1-ethyl-4-methyl-1H-pyrazol-5-yl, 5-isopropoxypyridin-2-yl, 6-(trifluoromethyl)pyridin-3-yl, 3,5-difluoropyridin-2-yl, 3,5-difluoropyridin-4-yl, 1-ethyl-4-cyano-1H-pyrazol-3-yl, 5-fluoropyridin-2-yl, 5-(difluoromethyl)pyridin-2-yl, 5-(trifluoromethyl)pyridine-2-yl, pyrazin-2-yl, 5,6-dimethylpyrazin-2-yl, 5-methylpyrazin-2-yl or 3-(trifluoromethyl)pyridazin-4-yl; or
f) 1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 2-methyl-[1,2,4]triazolo[1,5-a]pyridin-6-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, 2-methylbenzo[d]thiazol-5-yl, benzo[d]thiazol-6-yl, benzo[d]thiazol-7-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, 3-methylquinoxalin-6-yl, 3-methoxyquinoxalin-6-yl, quinoxalin-6-yl-2,3-d2, 1-ethyl-1H-indol-2-yl, 1-methyl-1H-benzo[d]imidazol-2-yl, 1-ethyl-1H-benzo[d]imidazol-2-yl, 1-propyl-1H-benzo[d]imidazol-2-yl, 1-ethyl-5-(trifluoromethyl)-1H-benzo[d]imidazol-2-yl, 1-ethyl-6-(trifluoromethyl)-1H-benzo[d]imidazol-2-yl, 1-ethyl-7-(trifluoromethyl)-1H-benzo[d]imidazol-2-yl, 1-methyl-1H-benzo[d]imidazol-6-yl, 3-ethyl-3H-imidazo[4,5-b]pyridin-2-yl, 1-ethyl-1H-imidazo[4,5-b]pyridin-2-yl, 3-cyclopropylquinoxalin-6-yl, 3-aminoquinoxalin-6-yl, 3-trifluoromethylquinoxalin-6-yl, 2-trifluoromethylquinoxalin-6-yl, 3-bifluoromethylquinoxalin-6-yl, 3-(1,1-bifluoroethyl)quinoxalin-6-yl, 2-deuterium-3-methylquinoxalin-6-yl, 2-deuterium-3-methoxyquinoxalin-6-yl, 3-methyl-5-methoxyquinoxalin-6-yl, 3-methyl-7-methoxyquinoxalin-6-yl, 3-methyl-5-trifluoromethylquinoxalin-6-yl, 3-methyl-7-trifluoromethylquinoxalin-6-yl, 1-ethyl-4,5,6,7-tetrahydro-1H-benzo[d]imidazol-2-yl, 2,3-dihydro-[1,4]dioxino[2,3-b]pyridin-6-yl, 2,3-dimethyl-quinoxalin-6-yl, 3-ethyl-quinoxalin-6-yl, 3-chloro-quinoxalin-6-yl, 4-methoxy-quinoxalin-6-yl, 3-(difluoromethyl)quinoxalin-6-yl, 3-(1,1-difluoroethyl)quinoxalin-6-yl, 3-cyclopropyl-quinoxalin-6-yl, 2-hydroxy-3-methylquinoxalin-6-yl, or 3-(methyl-d3)quinoxalin-6-yl; or
g) cyclobutyl, cyclopentyl, cyclohexyl, 2,3-dihydro-1H-inden-1-yl, 2,3-dihydro-1H-inden-2-yl, 1,2,3,4-tetrahydronaphthalen-1-yl, 1,2,3,4-tetrahydronaphthalen-2-yl, or 6,7,8,9-tetrahydro-5H-benzo[7]annulen-5-yl.

In some embodiments, Cy¹ is 2-(trifluoromethoxy)phenyl; 2-methoxyphenyl; 2-(methoxymethyl)phenyl; 2-(trifluoromethyl)phenyl; 4-fluoro-2-(methoxymethyl)phenyl; 4-fluoro-2-methoxyphenyl; 4-fluoro-2-(methoxymethyl)phenyl; 4-fluoro-2-methylphenyl; 2-bromo-4-fluorophenyl; 4-fluoro-2-(methylsulfonyl)phenyl; 4-methyl-2-(trifluoromethyl)phenyl; 2-chloro-4-fluorophenyl; 2,4-difluorophenyl; 2-ethoxy-4-fluorophenyl; 4-fluoro-2-isopropoxyphenyl; 4-fluoro-2-(trifluoromethoxy)phenyl; 2-(difluoromethoxy)-4-fluorophenyl; 2-(difluoromethyl)-4-fluorophenyl; 2-cyclopropyl-4-fluorophenyl; 4-fluoro-2-(1-hydroxyethyl)phenyl; 4-cyclopropyl-2-methoxyphenyl; 2-ethyl-4-fluorophenyl; 4-fluoro-2-(trifluoromethyl)phenyl; 2-methoxy-4-fluorophenyl; 2-(1,1-difluoroethyl)-4-fluorophenyl; 4-fluoro-3-(methoxymethyl)phenyl; 3-methyl-2-(trifluoromethyl)phenyl; 4-fluoro-2,6-dimethoxyphenyl; 2,4-difluoro-6-methoxyphenyl; 2,6-dichloro-4-fluorophenyl; 2,3-dihydrobenzo[b][1,4]dioxin-6-yl; 3,3-dimethyl-2,3-dihydrobenzo[b][1,4]dioxin-6-yl; 2,2-difluorobenzo[d][1,3]dioxol-4-yl; 3-methoxypyridin-2-yl; 2-methoxypyridin-3-yl; 2-(trifluoromethyl)pyridin-3-yl; 6-(difluoromethoxy)pyridin-3-yl; 3-methoxypyridin-4-yl; 5-fluoro-3-(trifluoromethyl)pyridin-2-yl; 5-chloro-1-ethyl-1H-imidazol-2-yl; 1-ethyl-2-(trifluoromethyl)-1H-imidazol-5-yl; 1-ethyl-5-(trifluoromethyl)-1H-pyrazol-4-yl; 5-ethyl-1-methyl-1H-pyrazol-4-yl; 1-methyl-5-(trifluoromethyl)-1H-pyrazol-4-yl; 5-(difluoromethyl)-1-methyl-1H-pyrazol-4-yl; 1-ethyl-3-(trifluoromethyl)-1H-pyrazol-5-yl; 3-chloro-1-ethyl-1H-pyrazol-5-yl; 1-ethyl-4-methyl-1H-pyrazol-5-yl; quinolin-3-yl; quinolin-2-yl; quinoxalin-6-yl; 3-methylquinoxalin-6-yl; 3-methylquinoxalin-6-yl; 3-methylquinoxalin-6-yl; 3-cyclopropylquinoxalin-6-yl; 3-aminoquinoxalin-6-yl; 3-trifluoromethylquinoxalin-6-yl; 3-bifluoromethylquinoxalin-6-yl; 3-(1,1-bifluoroethyl)quinoxalin-6-yl; 2-deuterium-3-methylquinoxalin-6-yl; 2-deuterium-3-methoxyquinoxalin-6-yl; 3-methyl-5-methoxyquinoxalin-6-yl; 3-methyl-7-methoxyquinoxalin-6-yl; 3-methyl-5-trifluoromethylquinoxalin-6-yl; 3-methyl-7-trifluoromethylquinoxalin-6-yl; quinoxalin-6-yl-2,3-d2; 1-ethyl-1H-indol-2-yl; 1-methyl-1H-benzo[d]imidazol-2-yl; 1-ethyl-1H-benzo[d]imidazol-2-yl; 1-propyl-1H-benzo[d]imidazol-2-yl; 1-ethyl-5-(trifluoromethyl)-1H-benzo[d]imidazol-2-yl; 1-ethyl-6-(trifluoromethyl)-1H-benzo[d]imidazol-2-yl; 1-ethyl-7-(trifluoromethyl)-1H-benzo[d]imidazol-2-yl; 1-methyl-1H-benzo[d]imidazol-6-yl; 3-ethyl-3H-imidazo[4,5-b]pyridin-2-yl; 1-ethyl-1H-imidazo[4,5-b]pyridin-2-yl; 1-ethyl-4,5,6,7-tetrahydro-1H-benzo[d]imidazol-2-yl; 3-methoxyquinoxalin-6-yl; 3-(trifluoromethyl)pyridin-4-yl; 4-cyclopropylphenyl; 4-methoxyphenyl; 4-fluorophenyl; 4-cyclopropyl-phenyl; 4-(trifluoromethyl)phenyl; 4-methylphenyl; 4-(difluoromethyl)phenyl; 4-isopropoxyphenyl; 2-fluoro-4-(trifluoromethyl)phenyl; 4-cyclopropyl-2-fluorophenyl; 2,4-difluorophenyl; 4-cyclopropyl-2-fluorophenyl; 4-fluoro-2-(trifluoromethyl)phenyl; 3-methoxy-4-(trifluoromethyl)phenyl; 4-fluoro-3-methoxyphenyl; 2,6-difluorophenyl; 2,6-difluoro-4-methoxyphenyl; 2,5-difluoro-4-methoxyphenyl; naphthalen-2-yl; 3,4-dihydro-2H-benzo[b][1,4]oxazin-6-yl; chroman-4-yl; chroman-6-yl; 4,4-difluorochroman-6-yl; 1,2,3,4-tetrahydronaphthalen-1-yl; 2,3-dihydro-1H-inden-1-yl; 5-isopropoxypyridin-2-yl; 6-isopropoxypyridin-3-yl; 6-(trifluoromethyl)pyridin-3-yl; 3,5-difluoropyridin-2-yl; 3,5-difluoropyridin-4-yl; 1-ethyl-4-cyano-1H-pyrazol-3-yl; quinolin-2-yl; isoquinolin-3-yl; isoquinolin-6-yl; isoquinolin-7-yl; 1,8-naphthyridin-2-yl; quinoxalin-6-yl; quinoxalin-2-yl; [1,2,4]triazolo[1,5-a]pyridin-7-yl; benzo[d]thiazol-2-yl; 2,3-dihydro-[1,4]dioxino[2,3-b]pyridin-6-yl; 3,3-dimethyl-2,3-dihydro-[1,4]dioxino[2,3-b]pyridin-6-yl; 4-(trifluoromethoxy)phenyl; 4-fluorophenyl; 4-acetamidophenyl; pyridin-2-yl; pyridin-3-yl; pyridin-4-yl; 5-fluoropyridin-2-yl; pyrazin-2-yl; pyrimidin-2-yl; quinolin-7-yl; 3-methylisoquinolin-7-yl; quinoxalin-6-yl; quinolin-6-yl; quinolin-7-yl; or pyrimidin-4-yl.

In some embodiments, the compound of formula (I) is a compound of formula (If)

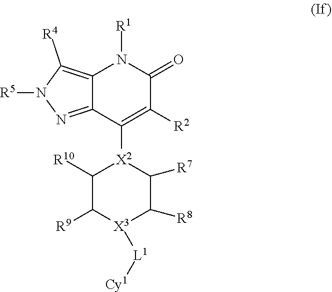

Wherein R¹, R², R⁴, R⁵, R⁷, R⁸, R⁹, R¹⁰, X², X³, L¹, Cy¹ are defined as in Formula (I).

### DETAILED DESCRIPTION OF THE DISCLOSURE

### Definitions

The following terms have the indicated meanings throughout the specification:

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups derived from an alkane by removal of one hydrogen atom from the same carbon atom, which comprises from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups. The alkyl group can be optionally enriched in deuterium, e.g., —CD₃, —CD₂CD₃ and the like.

The term “alkylene” refers to a hydrocarbon group selected from linear and branched saturated hydrocarbon groups derived from an alkane by removal of two hydrogen atoms from the same carbon atom, which comprises from 1 to 6, such as from 1 to 4, carbon atoms, further such as from 1 to 3, more further such as 1, 2 or 3 carbon atoms, include, but not limited to, methylene (—CH₂—), ethylene (—CH₂CH₂—), 1-methymethylene (—CH(CH₃)—), or trimethylene (—CH₂CH₂CH₂—).

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈ alkyl, haloC₁₋₆alkyl or halo C₁₋₄alkyl, but not limited to —CF₃, —CH₂Cl, —CH₂CF₃, —CCl₂, CF₃, and the like.

The term “alkyloxy” or “alkoxy” refers to an alkyl group as defined above attached to the parent molecular moiety through an oxygen atom. Examples of an alkyloxy, e.g., C₁₋₆alkyloxy or C₁₋₄ alkyloxy include, but not limited to, methoxy, ethoxy, isopropoxy, propoxy, n-butoxy, tert-butoxy, pentoxy and hexoxy and the like.

The term “amino” refers to —NH₂.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “deuterated” is used herein to modify a chemical structure or an organic group or radical, wherein one or more carbon-bound hydrogen(s) are replaced by one or more deuterium(s), e.g., “deuterated-alkyl”, “deuterated-cycloalkyl”, “deuterated-heterocycloalkyl”, “deuterated-aryl”, “deuterated-morpholinyl”, and the like. For example, the term “deuterated-alkyl” defined above refers to an alkyl group as defined herein, wherein at least one hydrogen atom bound to carbon is replaced by a deuterium. In a deuterated alkyl group, at least one carbon atom is bound to a deuterium; and it is possible for a carbon atom to be bound to more than one deuterium; it is also possible that more than one carbon atom in the alkyl group is bound to a deuterium.

The term “aryl” used alone or in combination with other terms refers to a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like.

In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” herein refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The term “optionally oxidized sulfur” used herein refers to —S—, SO or SO₂.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) 1H-pyrazolyl (such as 1H-pyrazol-3-yl, 1H-pyrazol-4-yl or 1H-pyrazol-5-yl), pyridyl or pyridinyl (such as 2-pyridyl, 3-pyridyl, or 4-pyridyl), cinnolinyl, pyrazinyl, pyrimidinyl (such as pyrimidin-2-yl, pyrimidin-4-yl, pyrimidin-5-yl or 2,4-pyrimidinyl, 3,5-pyrimidinyl), imidazolyl (such as 1H-imidazol-2-yl, 1H-imidazol-4-yl, 1H-imidazol-5-yl, or 2,4-imidazolyl), imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl (such as 1H-indol-2-yl, 1H-indol-3-yl, 1H-indol-4-yl, 1H-indol-5-yl, 1H-indol-6-yl or 1H-indol-7-yl), isoindolyl, indolinyl, oxadiazolyl (such as 1,2,3-oxadiazolyl, 1,2,4-oxadiazolyl, or 1,3,4-oxadiazolyl), phthalazinyl, pyrazinyl (such as pyrazin-2-yl), pyridazinyl, pyrrolyl, triazolyl (such as 1,2,3-triazolyl, 1,2,4-triazolyl, or 1,3,4-triazolyl), quinolinyl (such as quinolin-2-yl, quinolin-3-yl, quinolin-4-yl, quinolin-5-yl, quinolin-6-yl, or quinolin-7-yl), isoquinolinyl (such as isoquinolin-1-yl, isoquinolin-3-yl, isoquinolin-4-yl, isoquinolin-5-yl, isoquinolin-6-yl, isoquinolin-7-yl, or isoquinolin-8-yl), pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl (such as benzo[d]oxazol-2-yl, benzo[d]oxazol-4-yl, benzo[d]oxazol-5-yl, benzo[d]oxazol-6-yl or benzo[d]oxazol-7-yl), quinazolinyl, quinoxalinyl (such as quinoxalin-2-yl, quinoxalin-3-yl, quinoxalin-4-yl, quinoxalin-5-yl, quinoxalin-6-yl, quinoxalin-7-yl or quinoxalin-8-yl), naphthyridinyl (such as 1,8-naphthyridin-2-yl, 1,8-naphthyridin-3-yl, or 1,8-naphthyridin-4-yl), 2,3-dihydro-[1,4]dioxino[2,3-b]pyridinyl (such as 2,3-dihydro-[1,4]dioxino[2,3-b]pyridin-6-yl, 2,3-dihydro-[1,4]dioxino[2,3-b]pyridin-7-yl, or 2,3-dihydro-[1,4]dioxino[2,3-b]pyridin-8-yl), furopyridinyl, benzothiazolyl (such as benzo[d]thiazol-2-yl, benzo[d]thiazol-4-yl, benzo[d]thiazol-5-yl, benzo[d]thiazol-6-yl or benzo[d]thiazol-7-yl), benzo[d]imidazolyl (such as 1H-benzo[d]imidazol-2-yl, 1H-benzo[d]imidazol-4-yl, 1H-benzo[d]imidazol-5-yl, 1H-benzo[d]imidazol-6-yl or 1H-benzo[d]imidazol-7-yl), [1,2,4]triazolo[1,5-a]pyridinyl (such as [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, or [1,2,4]triazolo[1,5-a]pyridin-8-yl), 3H-imidazo[4,5-b]pyridinyl (such as 3H-imidazo[4,5-b]pyridin-2-yl, 3H-imidazo[4,5-b]pyridin-5-yl, 3H-imidazo[4,5-b]pyridin-6-yl or 3H-imidazo[4,5-b]pyridin-7-yl), 1H-imidazo[4,5-b]pyridinyl (such as 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl), [1,2,4]triazolo[1,5-a]pyridinyl (such as [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl or [1,2,4]triazolo[1,5-a]pyridin-8-yl), indazolyl (such as 1H-indazol-5-yl) and 5,6,7,8-tetrahydroisoquinoline.

Also, a “heteroaryl” fused with a “Heterocyclyl” is defined as “heteroaryl”.

“Heterocyclyl,” “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “monocyclic heterocyclyl” refers to monocyclic groups in which at least one ring member is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 3-membered/5-membered, 4-membered/5-membered, 4-membered/6-membered, 5-membered/5-membered, or 5-membered/6-membered mono-spiro heterocyclyl.

The term “fused heterocyclic group” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but none of the rings has a completely conjugated pi-electron system. Preferably, a fused heterocyclyl is 6 to 14-membered, and more preferably 7 to 10-membered. According to the number of membered rings, a fused heterocyclyl is divided into bicyclic, tricyclic, tetracyclic, or polycyclic fused heterocyclyl, preferably refers to bicyclic or tricyclic fused heterocyclyl, and more preferably 5-membered/5-membered, or 5-membered/6-membered bicyclic fused heterocyclyl. Representative examples of fused heterocycles include, but not limited to, the following groups octahydrocyclopenta[c]pyrrole (e.g., octahydrocyclopenta[c]pyrrol-2-yl), octahydropyrrolo[3,4-c]pyrrolyl, octahydroisoindolyl, isoindolinyl (e.g., isoindoline-2-yl), octahydro-benzo[b][1,4]dioxin.

The term “bridged heterocyclyl” refers to a 5- to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the titled stereoisomer contains no more than 35%, such as no more than 30%, further such as no more than 25%, even further such as no more than 20%, by weight of any other stereoisomer(s). In some embodiments, the term “substantially pure” means that the titled stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or flash column chromatography. Flash column chromatography can involve any number of methods including, for example: reverse-phase and normal phase; size exclusion; ion exchange; high, medium and low pressure liquid flash column chromatography methods and apparatus; small scale analytical; simulated moving bed (“SMB”) and preparative thin or thick layer flash column chromatography, as well as techniques of small scale thin layer and flash column flash column chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by flash column chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column. A single stereoisomer, e.g., a substantially pure enantiomer, may be obtained by resolution of the racemic mixture using a method such as formation of diastereomers using optically active resolving agents [Eliel, E. and Wilen, S. Stereochemistry of Organic Compounds. New York: John Wiley & Sons, Inc., 1994; Lochmuller C. H, et al. “Flash column chromatographic resolution of enantiomers: Selective review.” J. Chromatogr., 113 (3) (1975): pp. 283-302]. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer; Irving W, Ed. Drug Stereochemistry: Analytical Methods and Pharmacology. New York: Marcel Dekker, Inc., 1993. The absolute configuration of the chiral centers in a compound can be determined using methods known to one skilled in the art, e.g., single crystal X-ray crystallography or co-crystal formation of a compound of interest with the targeted proteins, sometime coupled with a spectroscopic technique, e.g., NMR spectroscopy. In some embodiments, the absolute configuration of chiral centers in a compound can be elucidated from the X-ray single-crystal structure of the compound. In some embodiments, the absolute configuration of chiral centers elucidated by the X-ray crystal structure of a compound can be used to infer the absolute configuration of the corresponding chiral centers in another compound or an intermediate obtained from the same or similar synthetic methodologies.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

“Selective inhibitory activity” or “selectivity” refers to the difference in the degree of inhibition against DGKα and DGKζ; the greater the degree of inhibition effected for a particular isoform relative to another isoform, the greater the selectivity the inhibitor exhibits for that particular isoform. In some embodiments, “a compound showing selective inhibitory activity of DGKα over DGKζ” refers a compound which shows an IC50 against DGKα is not larger than about 2000 nM with the ratio of IC50 against DGKζ and IC50 against DGKα larger than or equal to about 20; “a compound showing selective inhibitory activity of DGKζ over DGKα” refers a compound which shows an IC50 against DGKζ is not larger than about 2000 nM with the ratio of IC50 against DGKα and IC50 against DGKζ larger than or equal to about 20; and “a compound showing dual inhibitory activity” refers to a compound which shows inhibitory activities against both DGKα and DGKζ with IC50 no larger than 500 nM and the ratio of the two IC50 values no more than 20.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid Formulation such as tablets, powder, granule, capsules and the like, a liquid Formulation such as water or oil suspension or other liquid Formulation such as syrup, solution, suspension or the like; for parenteral administration, the pharmaceutical composition may be solution, water solution, oil suspension concentrate, lyophilized powder or the like. Preferably, the Formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All Formulations of the pharmaceutical composition disclosed herein can be produced by the conventional methods in the pharmaceutical field. For example, the active ingredient can be mixed with one or more excipients, then to make the desired Formulation. The “pharmaceutically acceptable excipient” refers to conventional pharmaceutical carriers suitable for the desired pharmaceutical Formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the claims which follow, unless the context requires otherwise, the term “comprise,” and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### Abbreviations

| | |
|---|---|
| Ac | Acetyl |
| AcOH | Acetic acid |
| Aq | Aqueous |
| Brine | Saturated aqueous sodium chloride solution |
| Bn | Benzyl |
| Boc | Tert-butyloxycarbonyl |
| Cbz | Benzyloxycarbonyl |
| CDI | N,N′-Carbonyldiimidazole |
| DMF | N,N-Dimethylformamide |
| Dppf | 1,1″-bis(diphenylphosphino)ferrocene |
| DBU | 1,8-diazabicyclo[5.4.0]undec-7-ene |
| DCE | 1,2-dichloroethane |
| DCM | Dichloromethane |
| DHP | 3,4-dihydro-2H-pyran |
| DIPEA | N,N-diisopropylethylamine |
| DMAP | 4-N,N-dimethylaminopyridine |
| DMB | 2,4-dimethoxybenzyl |
| DMB-Cl | 1-(chloromethyl)-2,4-dimethoxybenzene |
| DMF | N,N-dimethylformamide |
| DMSO | Dimethyl sulfoxide |
| EA | Ethyl acetate |
| eq | Equivalent |
| g | Grams |
| HATU | O-(7-Azabenzotriazol-1-yl)-N,N,N′,N′-tetramethyluronium |
| | hexafluorophosphate |
| HCl | Hydrochloric acid |
| HPLC | High-performance liquid flash column chromatography |
| IPA | Isopropyl alcohol |
| i-PrOH | Isopropyl alcohol |
| KHMDS | Potassium Bis(trimethylsilyl)amide |
| LIHMDS | Lithium bis(trimethylsilyl)amide |
| mg | Milligrams |
| mL | Milliliters |
| mmol | Millimole |
| MeCN | Acetonitrile |
| MeOH | Methanol |
| Min | Minutes |
| MS | Mass spectrum |
| NMR | Nuclear magnetic resonance |
| NCS | N-Chlorosuccinimide |
| NBS | N-Bromosuccinimide |
| NaHMDS | Sodium bis(trimethylsilyl)amide |
| OTf | Trifluoromethanesulfonate |
| Pd/C | Palladium on carbon |
| PE | Petroleum ether |
| PMB | 4-Methoxybenzyl |
| PMB-Cl | 1-(chloromethyl)-4-methoxybenzene |
| RT | room temperature |
| SEM | 2-trimethylsilylethoxymethoxy(2- |
| | (chloromethoxy)ethyl)trimethylsilane |
| TBSCl | tert-Butyldimethylsilyl chloride |
| tBuXPhos | Bis(1,1-dimethylethyl)[2′,4′,6′-tris(1- |
| | methylethyl)[1,1′-bipheny]]-2-yl]phosphine |
| tBuXPhos | Methanesulfonato(2-di-t-butylphosphino-2′,4′,6′-tri-i-propyl- |
| Pd G3 | 1,1′-biphenyl)(2′-amino-1,1′-biphenyl-2-yl)palladium(II) |
| TEA | Triethanolamine |
| TFA | Trifluoroacetic acid |
| THF | Tetrahydrofuran |
| THP | tetrahydropyran3,4-dihydro-2H-pyran |
| TLC | thin layer flash column chromatography |
| XPhos | 2-Dicyclohexylphosphino-2′,4′,6′-triisopropylbiphenyl |

### General Synthetic Schemes

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica flash column chromatography.

Chiral analytic HPLC was used for the retention time analysis of different chiral examples, the conditions were divided into the methods as below according to the column, mobile phase, solvent ration used. Preparation of homochiral examples may be carried out by techniques known to one skilled in the art. The absolute stereochemistry was not assigned at the newly formed carbon-nitrogen bond.

The compounds disclosed herein can be prepared by following Scheme I to V.

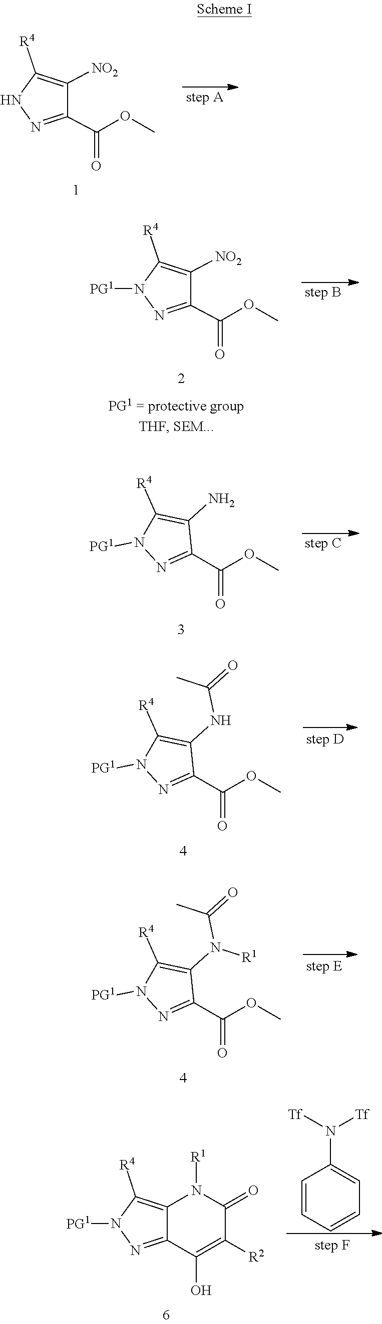

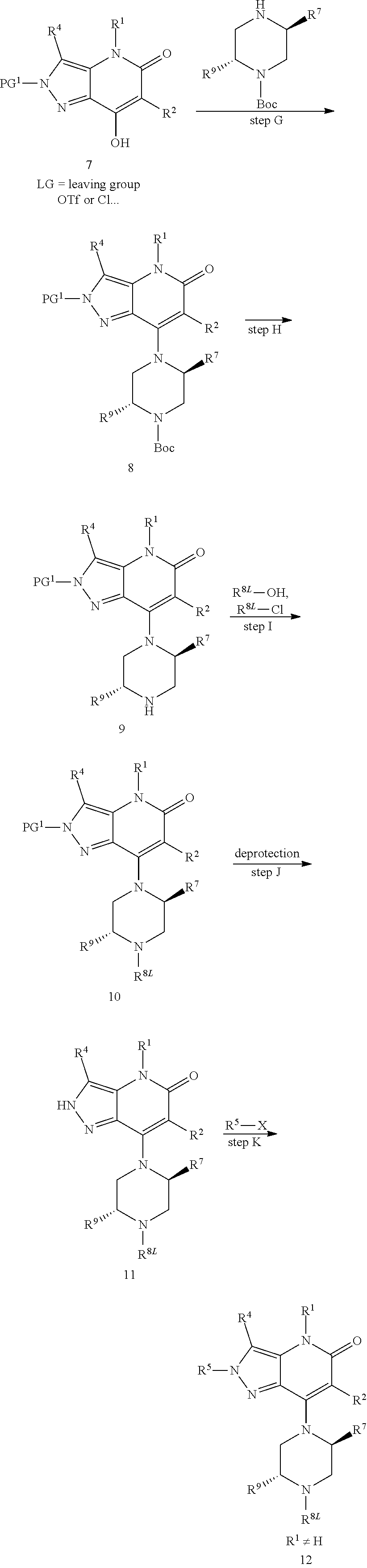

wherein the substitutions from R¹ to R⁹ and R^{8L} (corresponding to L¹-Cy¹) are as defined as mentioned in Formula (I).

In scheme I, a commercially available Compound 1 is protected with THP or SEM to give Compound 2. Compound 2 is reduced under reducing conditions (such as Pd—C/H₂, or Fe powder/NH₄Cl) to form Compound 3 as an amine. Compound 3 is acetylated by acetyl chloride or acetic anhydride to give Compound 4, and then reacted with appropriate R¹—X/R¹-OTf under basic condition (such as Cs₂CO₃, NaH, or t-BuOK) to give Compound 5. Compound 5 is further cyclized under basic condition (such as LiHMDS, NaHMDS, or KHMDS) to give Compound 6. Compound 6 is reacted with trifluoromethulfonate reagent (such as Tf₂O, or PhNTf₂) or phosphoryl chloride to give Compound 7. Compound 7 is reacted with the appropriate chiral secondary amine by nucleophilic aromatic substitution reaction to give Compound 8. The protected groups on the amine of Compound 8 are removed to give Compound 9 under acid condition (such as TFA or 4M solution of HCl in 1,4-dioxane). Tertiary amines Compound 10 is prepared by N-alkylation of secondary amines compound 9 by treatment with alkylating agents (such as alkylhalides or sulfonates) or reductive alkylation with aldehydes or ketones, the most frequently used procedures via a phosphonium salt mediated alkylation of amines with corresponding alcohols (Florencio Zaragoza and Henrik Stephensen, J. Org. Chem, 2001, 66, 2518-2521). Compound 10 is deprotected using acid condition (such as TFA or 4M solution of HCl in 1,4-dioxane) to give Compound 11. Routine reaction methods, such as alkylation, alkenylation reaction, acylation reaction or sulfonylation reaction of Compound 11 with corresponding alkenyl borate using copper-catalyzed Chan-Lam reaction or corresponding alkyl halides under basic condition (such as Cs₂CO₃, NaH, or t-BuOK), corresponding alkyl sulfonyl chloride/aliphatic acyl chloride using condensation reaction condition (such as Et₃N/HATU) to afford a compound of Formula 12 (wherein R² is H), and the halogenation of the compound of Formula 12 (wherein R² is H) with electrophilic halogenating reagents (such as NBS, NCS, or selectfluor) to afford a compound of Formula 12 (wherein R² is F, Cl, or Br), then the compound of Formula I (wherein R² is Br) also can be used to produce a compound of Formula 12 (wherein R² is CN, or Me) by normally Pd-catalyst coupling reaction with Zn(CN)₂ and 2,4,6-Trimethylboroxin.

Compound 10 disclosed herein can also be prepared by the following Scheme II.

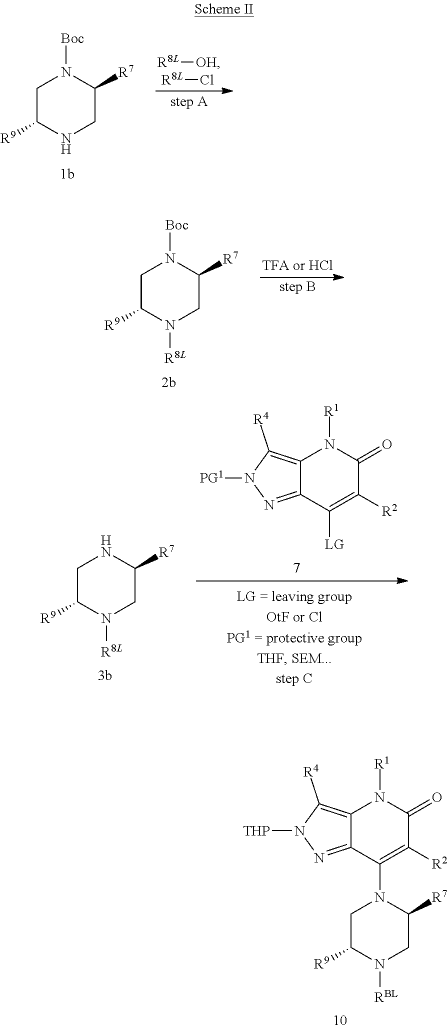

In Scheme II, Compound 2b is prepared by N-alkylation of secondary amines Compound 1b by treatment with alkylating agents (such as alkyl halides or sulfonates) or reductive alkylation with aldehydes or ketones, the most frequently used procedures via a phosphonium salt mediated alkylation of amines with corresponding alcohols. Compound 2b is deprotected using acid conditions (such as TFA or 4M solution of HCl in 1,4-dioxane) to give Compound 3b. Compound 3b is reacted with the Compound 7 by nucleophilic aromatic substitution reaction to give Compound 10.

The compound of Formula 12 disclosed herein also can be prepared by following Scheme III.

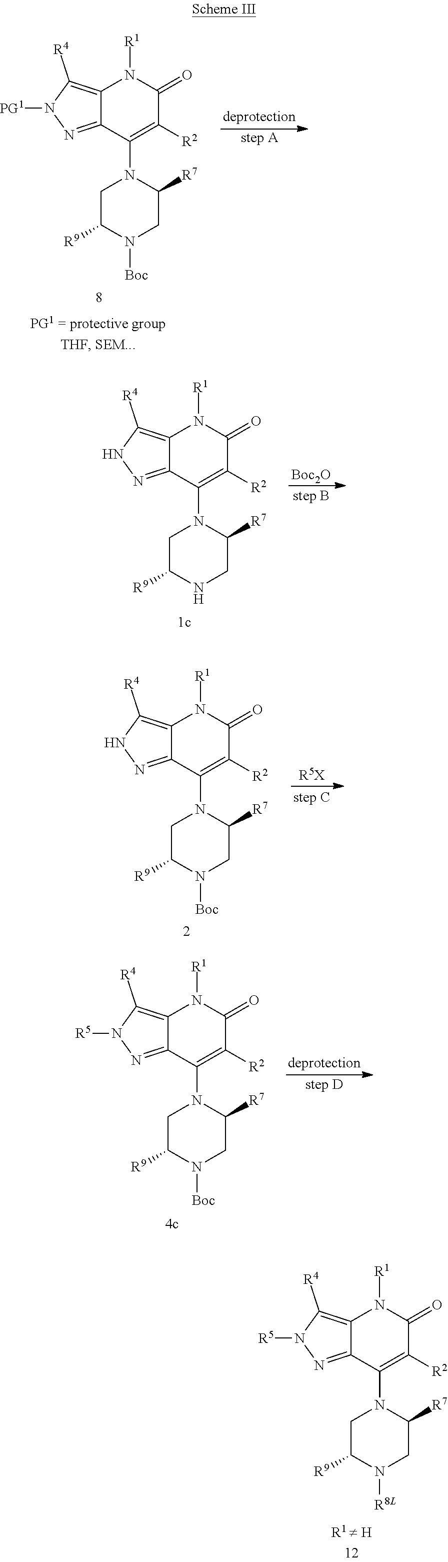

In Scheme III, the protected groups on the amine of Compound 8 are removed to give Compound 1c under acid conditions (such as TFA or 4M solution of HCl in 1,4-dioxane), Compound 1c is protected selectively with Boc₂O on the nitrogen-atoms of piperazine to give Compound 2c, alkylation reaction of compound 2c with corresponding alkyl halides under basic condition (such as Cs₂CO₃, NaH, or t-BuOK), to afford Compound 3c, which is deprotected using acid condition (such as TFA or 4M solution of HCl in 1,4-dioxane) to give Compound 4c, the Compound of Formula I was prepared by N-alkylation of secondary amines Compound 4b by treatment with alkylating agents (such as alkylhalides, sulfonates, etc.) or reductive alkylation with aldehydes or ketones, the most frequently used procedures via a phosphonium salt mediated alkylation of amines with corresponding alcohols.

The Compound 10d disclosed herein also can be prepared by following Scheme IV.

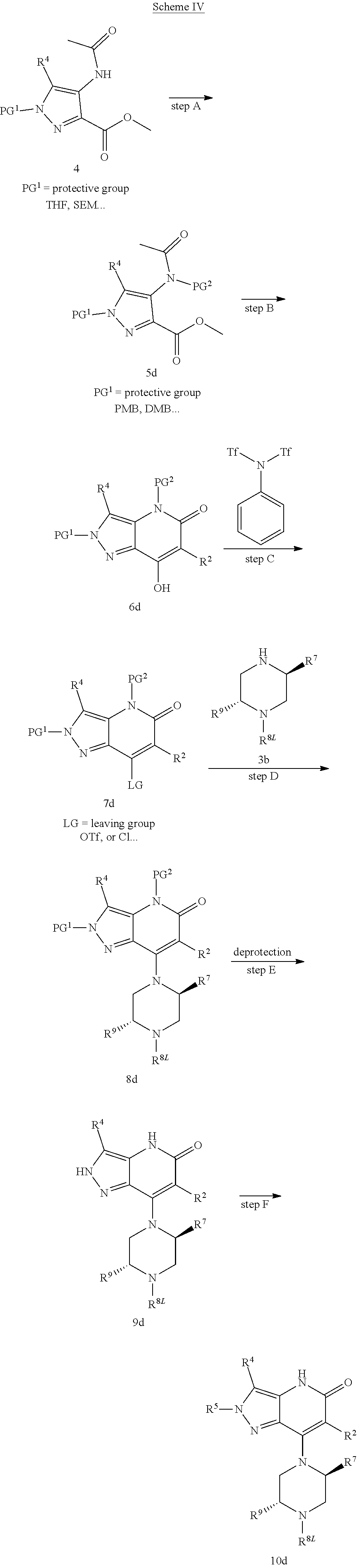

In Scheme IV, installation of PG² proceeded through alkylation reaction of compound 4 with appropriate R¹X (such as PMB-Cl, or DMB-Cl) under basic condition (such as Cs₂CO₃, or NaH) to give Compound 5d; which is cyclized under basic condition (such as lithium bis(trimethylsilyl)amide, sodium bis(trimethylsilyl)amide, potassium bis(trimethylsilyl)amide . . . ) to give Compound 6d, which subsequently react with trifluoromethulfonate reagent (such as trifluoromethanesulfonic anhydride, N-phenylbis(trifluoromethanesulfon)imide) or phosphoryl chloride to give Compound 7d, which is reacted with the corresponding secondary amine 3b by nucleophilic aromatic substitution reaction to give Compound 8d. The protective groups of Compound 8d are removed under strong acid condition (such as TfOH, or TFA) to give Compound 9d, which undergoes selective N-alkylation on pyrazole of Compound 9d by treatment with corresponding alkylhalides providing the compound of Formula 10d.

The compound of Formula 10A disclosed herein can also be prepared by following Scheme V.

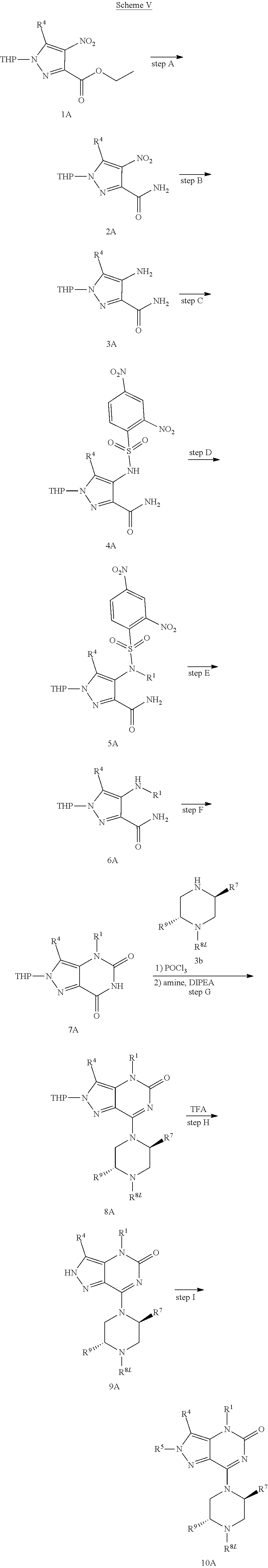

In Scheme V, commercially available Compound 1A via aminolysis reaction to afford Compound 2A which undergoes reduction providing Compound 3A with an appropriate reducing condition (such as Pd—C/H₂, or Fe powder/NH₄Cl). Compound 3A subsequently react corresponding sulfonyl chloride under basic condition (such as Et₃N, or pyridine) to afford Compound 4A, which react with appropriate R¹X/R¹OTf under basic condition (such as K₂CO₃, Cs₂CO₃, or NaH) to give Compound 5A. The protected group on the amine of Compound 5A is removed by 2-mercaptoacetic acid to give Compound 6A. Treatment of Compound 6A with 1,1′-carbonyldiimidazole under basic condition (such as NaH) afford cyclization Compound 7A, installation of the amine moiety 3b proceed through chlorination with phosphoryl chloride to give the chlorinated intermediate which is reacted with 3b to prevent hydrolysis giving Compound 8A, which is deprotected using acid condition (such as TFA or 4M solution of HCl in 1,4-dioxane) to give Compound 9A, and alkylation reaction of Compound 9A with corresponding alkyl halides under basic condition (such as Cs₂CO₃, or NaH) to afford the compound of Formula 10A.

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for flash column chromatography and silica gel (GF254) for thin-layer flash column chromatography (TLC) are commercially available from Tsingdao Haiyang Chemical Co., Ltd. or Yantai Chemical Co., Ltd. of China; all are eluted with petroleum ether (60-90° C.)/ethyl acetate (v/v), and visualized by iodine or the solution of molybdphosphoric acid in ethanol unless otherwise specified. All extraction solvents, unless otherwise specified, are dried over anhydrous Na₂SO₄.

¹H NMR spectra are recorded on Bruck-400 nuclear magnetic resonance spectrometer with TMS (tetramethylsilane) as the internal standard. LC/MS data are recorded by using Agilent1100 High Performance Liquid Flash column chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw®. Single crystal X-ray crystallography is used to elucidate the absolute configuration of a chiral center in a compound. For example, the compounds disclosed herein, e.g., Compound A2a, Compound A2b, Compound A22a, Compound A22b, Compound A133, Compound A134, Compound A269c, Compound A269d, Compound A274, Compound A301, Compound A302, Compound A303, Compound A317, Compound A318, Compound A319 or Compound A336, were determined to have the desired configurations by Single crystal X-ray crystallography.

### Synthesis

### Preparative HPLC Conditions (Method A)

| | |
|---|---|
| Column | Phenomenex Gemini NX-C18, 150 × 21.2 mm, 5 μm |
| Column Temp. | R.T. |
| Detection | DAD, UV λ = 214/254 nm |
| Wavelength | |
| Run Time | 17.0 min |
| Flow Rate | 20.0 mL/min |
| Mobile Phase A | 0.1% FA—H₂O (v/v) |
| Mobile Phase B | 0.1% FA—CH₃CN (v/v) |

### Preparative HPLC Conditions (Method B)

| Column | Waters XSelect CSH C18, | |
|---|---|---|
| | 150 × 19 mm, 5 μm | |
| Column Temp. | R.T. | |
| Detection Wavelength | DAD, UV λ = 214/254 nm | |
| Run Time | 17.0 | min |
| Flow Rate | 17 | mL/min |

| | |
|---|---|
| Mobile Phase A | 0.03% NH₃•H₂O—H₂O (v/v) |
| Mobile Phase B | CH₃CN |

### Intermediate 1: 6-(1-((2R,5S)-2,5-dimethylpiperazin-1-yl)ethyl)quinoxaline

### Step A: quinoxaline-6-carboxylic acid methoxy-methyl-amide

To a solution of quinoxaline-6-carboxylic acid (52.2 g, 0.3 mol) in DCM (1 L) were added HATU (136.8 s, 0.36 mmol), DIPEA (155 g, 1.2 mol) and N,N-dimethyl-hydroxylamine hydrochloride salt (35 g, 0.36 mol). The mixture was stirred at RT for 4 hours. The reaction was diluted with DCM and washed with water. The organic layer was separated, dried by Na₂SO₄, filtered, and concentrated to dryness. The resulting residue was purified by flash column chromotography to give the titled compound (67 g, 99%). ¹H NMR (400 MHz, DMSO): δ 9.05 (s, 2H), 8.31 (d, J=L8 Hz, 1H), 8.18 (d, J=8.6 Hz, 1H), 8.03 (d, J=8.7, 1.8 Hz, 1H), 3.59 (s, 3H), 3.36 (s, 3H) ppm, MS: M/c 218 (M+1)⁺.

### Step B: 1-(quinoxalin-6-yl)ethan-1-one

To a solution of quinoxaline-6-carboxylic acid methoxy-methyl-amide (67 g, 0.3 mol) in THF (500 mL) at 0° C. was added methyl magnesium bromide (133 mL, 3M, 0.39 mol). The reaction mixture was stirred at 0° C. for 2 hours, stirred at room temperature for 2 hours. The reaction was quenched by adding aqueous NH₄Cl. The resulting mixture was extracted with EA, and then concentrated by using a rotary evaporator, to give a residue. The resulting residue was purified by flash column chromatography (PE:EA=1:3) to give the titled compound (41 g, 77%). MS: M/e 17 (M+)⁺.

### Step C: 1-(quinoxalin-6-yl)ethan-1-ol

NaBH₄ (6.7 g, 0.17 mol) was added to 1-(quinoxalin-6-yl)ethan-1-one (40 g, 0.23 mol) in EtOH (200 ml) at 0° C. for 1 hour. The reaction was quenched by adding water. The mixture was extracted with EA and washed with brine. The organic layer was separated, dried by Na₃SO₄, filtered and concentrated to dryness. The resulting residue was purified by flash column chromatography to give the titled compound (29 g, 62%). ¹H NMR (400 MHz, DMSO) δ 8.93 (d, J=9.0 Hz, 2H), 8.11-8.00 (m, 2H), 7.86 (t, J=12.6 Hz, 1H), 5.52 (d, J=4.2 Hz, 1H), 5.07-4.92 (n, 1H), 1.44 (d, J=6.4 Hz, 3H) ppm. MS: M/e 175 (M+1)⁺.

### Step D: tert-butyl (2S,5R)-2,5-dimethyl-4-(1-(quinoxalin-6-yl)ethyl)piperazine-1-carboxylate

A solution of 1-(quinoxalin-6-yl)ethan-1-ol (22 g, 0.13 mol), tert-butyl (2S,5R)-2,5-dimethylpiperazine-1-carboxylate (32 g, 0.15 mol), (cyanomethyl)trimethylphosphonium iodide (46 g, 0.19 mol) and DIPEA (65 g, 0.5 mol) in CH₃CN (200 ml). The mixture was stirred at 105° C. for overnight and cooled to room temperature, and then the solvent was removed under reduced pressure. The resulting residue was diluted by adding water, extracted with EA and washed with brine. The organic layer was separated, dried by Na₂SO₄, filtered and concentrated to dryness. The resulting residue was purified by flash column chromatography to give the titled compound (32 g, 68%), ¹H NMR (400 MHz, DMSO): 8.92 (dq, J=5.4, 1.8 Hz, 2H), 8.09 d, J=9.0 Hz, 1H), 8.01 (dd, J=6.0, 1.3 Hz, 1H), 7.95-7.89 (m, 1H), 4.19-4.01 (m, 1H) 3.98-3.84 (m, 1H), 3.74-3.63 (m, 1H), 3.41 (d, J=12.3 Hz, 1H), 3.33-3.05 (m, 1H), 2.74-2.65 (m, 1H), 1.98 (d, J=14.2 Hz, 1H), 1.39 (d, J=2.4 Hz, 9H), 1.33-1.23 (m, 4H), 1.20-1.03 (m, 2H), 1.00-0.83 (m, 3H) ppm. MS: M/e 371 (M+1)⁺.

### Step E: 6-(1-((2R,5S)-2,5-dimethylpiperazin-1-yl)ethyl)quinoxaline (Intermediate 1)

TFA (90 mL) was added to tert-butyl (2S,5R)-2,5-dimethyl-4-(1-(quinoxalin-6-yl)ethyl)piperazine-1-carboxylate (32 g) in DCM (300 ml). The mixture was stirred at room temperature for 4 hours. The solvent was removed under reduced pressure. The resulting residue was diluted with water and extracted with EA. The aqueous layer was adjusted pH to 12-13 with saturated Na₂CO₃ solution, and then extracted with DCM. The combined organic layers were washed with brine, and then the organic layer was separated, dried by Na₂SO₄, filtered, and concentrated to give Intermediate 1.

The resulting residue Intermediate 1 (18 g) as a mixture of diastereomers was performed chiral resolution by C18 column (mobile phase B: MeOH; mobile phase A: H₂O (0.5 NH₃H₂O)) to give Intermediate 1a (the earlier peak, 8 g, 34%) and Intermediate 1b (the later peak, 8 g, 34%).

Intermediate 1a: ¹H NMR (400 MHz, DMSO-d₆) δ 8.93 (dd, J=6.8, 1.7 Hz, 2H), 8.14-8.02 (n 1H), 7.91 (s, 1H), 7.82 (dd, J=0.7, 1.7 Hz, 1H), 4.46 (q, J=7.0 Hz, 1H), 2.97 dd, J=10.7, 2.3 Hz, 1H), 2.72-2.59 (m, 2H), 2.37-2.29 (m, 1H), 2.05-2.01 (m, 1H), 1.83 (s, 1H), 1.51 (d, J=1 Hz, 3H), 1.42 (t, J=10.3 Hz, 11H), 1.08 (d, J=6.0 Hz, 3H), 0.86 (d, J=6.3 Hz, 3H) ppm. MS: M/e 271 (M+1)⁺.

Intermediate 1b: ¹H NMR (400 MHz, CD₃OD) δ 8.90 (dd, J=5.5, 1.7 Hz, 2H), 8.15 (s, 1H), 8.12-8.05 in, 2H), 4.82 (s, 1H), 3.50-3.40 (m, 1H) 3.23 (s, 2H), 3.10-3.00 (m, 1H), 2.67-2.50 (m, 2H), 1.58 (t, J=6.0 Hz, 3H), 1.44 (t, J=5.6 Hz, 3H), 1.16 (d, J=6, 5, 2.6 Hz, 3H) ppm. MS: M/e 271 (M+1)⁺.

Intermediate 2: 4-methyl-5-oxo-2-(tetrahydro-2H-pyran-2-yl)-4,5-dihydro-2H-pyrazolo[4,3-b]pyridin-7-yl trifluoromethanesulfonate

### Step A: methyl 4-nitro-1-(tetrahydro-2H-pyran-2-yl)-1H-pyrazole-3-carboxylate

To a solution of methyl 4-nitro-1H-pyrazole-3-carboxylate (85.5 g, 0.5 mol) and TsOH·H₂O (9.5 g, 0.05 mol) in THF (0.6 L) was added DHP (84 g, 1 mol) in some portions. The reaction was stirred at 80° C. for 16 hours. Then the mixture was cooled to room temperature and concentrated to dryness. The resulting residue was purified by flash column chromatography to give the titled compound (121 g, 94%). ¹HNMR (400 MHz, CDCl₃): δ 8.38 (s, 1H), 5.43 (dd, J=2.4, 8.8 Hz, 1H), 4.15-4.05 (m, 1H), 3.99 (s, 3H), 3.78-3.68 (m, 1H), 2.26-2.17 (m, 1H), 2.01-1.85 (m, 2H), 1.76-1.60 (m, 3H) ppm. MS: M/e 278 (M+23)⁺.

### Step B: methyl 4-amino-1-(tetrahydro-2H-pyran-2-yl)-1H-pyrazole-3-carboxylate

To a solution of methyl 4-nitro-1-(tetrahydro-2H-pyran-2-yl)-1H-pyrazole-3-carboxylate (64 g & 57 g, two batches) in MeOH (300 mL) was added (6 g). The mixture was stirred at room temperature under H₂ atmosphere, filtered and washed with MeOH. The combined filtrates were concentrated to give the titled compound (96 g, 90%). ¹HNMR (400 MHz, CDCl₃): δ 7.21 (s, 1H), 5.33 (dd, J=2.4, 9.6 Hz, 1H), 4.11-4.02 (m, 1H), 3.92 (s, 3H), 3.71-3.62 (m, 1H), 2.12-1.90 (m, 3H), 1.76-1.52 (m, 3H) ppm. MS: M/e 226 (M+1)⁺.

### Step C: methyl 4-acetamido-1-(tetrahydro-2H-pyran-2-yl)-1H-pyrazole-3-carboxylate

To a solution of methyl 4-amino-1-(tetrahydro-2H-pyran-2-yl)-1H-pyrazole-3-carboxylate (96 g, 426 mmol) in pyridine (0.4 L) was added Ac₂O (60 mL) dropwise to keep the temperature below 20° C. The resulting mixture was stirred at RT for 3 hours and concentrated to dryness. The residue was recrystallized from PE/EA to give the titled compound (84 g, 74%). ¹HNMR (400 MHz, CDCl₃): δ 8.99 (s, 1H), 8.43 (s, 1H), 5.46-5.36 (m, 1H), 4.12-4.03 (m, 1H), 3.96 (s, 3H), 3.74-3.60 (m, 1H), 2.20 (s, 3H), 2.14-1.96 (m, 3H), 1.75-1.54 (m, 3H) ppm. MS: M/e 268 (M+1)⁺.

### Step D: methyl 4-(N-methylacetamido)-1-(tetrahydro-2H-pyran-2-yl)-1H-pyrazole-3-carboxylate

To a solution of methyl 4-acetamido-1-(tetrahydro-2H-pyran-2-yl)-1H-pyrazole-3-carboxylate (84 g, 314.6 mmol) in DMF (0.5 L) was added Cs₂CO₃ (306 g, 943 mmol), followed by CH₃I (134 g, 943 mmol). The reaction was stirred at room temperature (RT) for 16 hours. The reaction mixture was filtered through Celite, washed with DCM. The filtrate was concentrated, and then the obtained residue was diluted with water, extracted with DCM (400 mL×2), washed with brine, dried over Na₂SO₄, filtered, and concentrated to give the titled compound (92 g, crude). ¹HNMR (400 MHz, CDCl₃): δ 7.67 (s, 1H), 5.47-5.36 (m, 1H), 4.16-4.03 (m, 1H), 3.92 (s, 3H), 3.77-3.65 (m, 1H), 3.18 (s, 3H), 2.23-2.12 (m, 1H), 2.09-1.90 (m, 2H), 1.87 (s, 3H), 1.80-1.56 (m, 3H) ppm MS: M/e 282 (M+1)⁺.

### Step E: 7-hydroxy-4-methyl-2-(tetrahydro-2H-pyran-2-yl)-2,4-dihydro-5H-pyrazolo[4,3-b]pyridin-5-one

To a solution of methyl 4-(N-methylacetamido)-1-(tetrahydro-2H-pyran-2-yl)-1H-pyrazole-3-carboxylate (88 g, 313 mmol) in THF (0.8 L) was added dropwise a solution of L1HMDS (˜500 mL, 1 mol/L, ˜1.6 eq) at −70° C. (A suspension was formed). The reaction was stirred for 0.5 hour, quenched with water, and warmed to RT slowly. The resulting mixture was extracted with EA. The water phase was collected, treated with citric acid to pH 3-4, and stirred at RT for 0.5 hours to give a suspension. The suspension was filtered, dried to give the titled compound (45.5 g). ¹HNMR (400 MHz, DMSO-d₆) δ 11.24 (s, 1H), 8.07 (s, 1H), 5.65 (s, 1H), 5.51 (dd, J=2.4 Hz, 10.0 Hz, 1H), 4.00-3.88 (m, 1H), 3.72-3.61 (m, 1H), 3.31 (s, 3H), 2.19-2.05 (m, 1H), 2.03-1.89 (m, 2H), 1.80-1.64 (m, 1H), 1.61-1.50 (m, 2H) ppm. MS: M/e 250 (M+1)⁺.

### Step F: 4-methyl-5-oxo-2-(tetrahydro-2H-pyran-2-yl)-4,5-dihydro-2H-pyrazolo[4,3-b]pyridin-7-yl trifluoromethanesulfonate (Intermediate 2)

To a solution of 7-hydroxy-4-methyl-2-(tetrahydro-2H-pyran-2-yl)-2,4-dihydro-5H-pyrazolo[4,3-b]pyridin-5-one (49.8 g, 200 mmol) in THF (0.5 L) was added K₂CO₃ (55 g, 400 mmol), followed by 1,1,1-trifluoro-N-phenyl-N-((trifluoromethyl)sulfonyl)methanesulfonamide (100 g, 943 mmol). The reaction was stirred at room temperature for 16 hours. The reaction mixture was filtered through Celite, and washed with EA. The filtrate was concentrated under reduced pressure. The resulting residue was diluted with water, washed with brine, dried by Na₂SO₄, filtered and concentrated to dryness. The resulting residue was purified by flash column chromatography and furtherly subjected to slurry with EA/PE to give the titled compound (70 g, 90%). ¹HNMR (400 MHz, CDCl₃) δ 7.65 (s, 1H), 6.58 (s, 1H), 5.58 (dd, J=2.8 Hz, 8.0 Hz, 1H), 4.11-4.00 (m, 1H), 3.82-3.70 (m, 1H), 3.52 (s, 3H), 2.26-1.96 (m, 3H), 1.81-1.61 (m, 3H) ppm. MS: M/e 382 (M+1)⁺.

### Intermediate 3: 7-((2S,5R)-2,5-diethylpiperazin-1-yl)-4-methyl-2-(tetrahydro-2H-pyran-2-yl)-2,4-dihydro-5H-pyrazolo[4,3-b]pyridin-5-one

### Step A: methyl (R)-2-(benzylamino)butanoate

To a solution of methyl (R)-2-aminobutanoate (100.0 g, 0.85 mol) in CH₃CN (1000 mL) was added benzyl bromide (146.1 g, 0.85 mol) at 0° C. under N₂ atmosphere. The reaction was stirred at room temperature overnight and concentrated under reduced pressure. The resulting residue was dissolved in EA (1000 mL) and washed with water (1000 mL×3). The organic layers were concentrated under reduced pressure. The resulting residue was purified by flash column chromatography (PE:EA=10:1) to give the titled compound (106 g, 60%). MS: M/e 208 (M+1)⁺.

### Step B: methyl (R)-2-((S)—N-benzyl-2-((tert-butoxycarbonyl)amino)butanamido)butanoate

To a solution of methyl (R)-2-(benzylamino)butanoate (117.0 g, 0.56 mol), (S)-2-((tert-butoxycarbonyl)amino)butanoic acid (170.5 g, 0.84 mmol) and 4-Methylmorpholine (113.1 g, 1.12 mmol) in DCM (2000 mL) was added HATU (319.0 g, 0.84 mmol) at 0° C. The reaction was stirred at room temperature overnight, and quenched by water and washed with water (1500 mL×2). The organic layers were concentrated under reduced pressure. The resulting residue was purified by flash column chromatography (PE:EA=10:1) to give the titled compound product (178 g, 80%). MS: M/e 393 (M+1)⁺.

### Step C: methyl (R)-2-((S)-2-amino-N-benzylbutanamido)butanoate

To a solution of methyl (R)-2-((S)—N-benzyl-2-((tert-butoxycarbonyl)amino)butanamido)butanoate (178 g, 0.45 mol) in 1,4-dioxane (100 mL) was added HCl (400 mL, 4 M in 1,4-dioxane) at room temperature.

The resulting mixture was stirred at room temperature for another 2 hours, and concentrated under vacuum to give the crude product (200 g, crude). MS: M/e 293 (M+1)⁺.

### Step D: (3S,6R)-1-benzyl-3,6-diethylpiperazine-2,5-dione

To a solution of methyl (R)-2-((S)-2-amino-N-benzylbutanamido)butanoate (200 g, crude) in EA (1000 mL) was added aq. NaHCO₃ (300 mL) at room temperature. The reaction mixture was stirred at room temperature for another 2 hours. The organic layers were concentrated under reduced pressure. The resulting residue was triturated with MTBE to give the titled compound (61 g, 52% for 2 steps, ee: 97%). MS: M/e 261 (M+1)⁺.

### Step E: (2R,5S)-1-benzyl-2,5-diethylpiperazine

To a solution of LiAlH4 (26.5 g, 0.69 mol) in THF (1000 mL) was added slowly (3S,6R)-1-benzyl-3,6-diethylpiperazine-2,5-dione (61.0 g, 0.23 mol) in THF (500 mL) at 0° C. The resulting mixture was stirred at room temperature for 2 hours, then stirred at 80° C. for overnight. The reaction was quenched by water (27 mL) slowly at 0° C. Then, 1N aqueous NaOH solution (54 mL) and water (81 mL) was added sequentially. The resulting mixture was stirred for 2 hours. The white precipitates that formed was removed by filtration. The filter cake was washed with EA (500 mL). The combined filtrates were evaporated. The resulting residue was dissolved in toluene. The solvent was removed under vacuum to dryness to afford the titled compound (51 g, 95%). MS: M/e 233 (M+1)⁺.

### Step F: 7-((2S,5R)-4-benzyl-2,5-diethylpiperazin-1-yl)-4-methyl-2-(tetrahydro-2H-pyran-2-yl)-2,4-dihydro-5H-pyrazolo[4,3-b]pyridin-5-one

A mixture of Intermediate 2 (3.8 g, 10 mmol), (2R,5S)-1-benzyl-2,5-diethylpiperazine (3.6 g, 15 mmol) and DIPEA (6.4 g, 50 mmol) in CH₃CN (50 mL) was heated to 105° C. for overnight under N₂ atmosphere. The solvent was removed under vacuum. The crude product was purified by flash column chromatography (DCM:MeOH=15:1) to give the titled compound (4.2 g, 90%). MS: M/e 464 (M+1)⁺.

### Step G: 7-((2S,5R)-2,5-diethylpiperazin-1-yl)-4-methyl-2-(tetrahydro-2H-pyran-2-yl)-2,4-dihydro-5H-pyrazolo[4,3-b]pyridin-5-one (Intermediate 3)

A mixture of 7-((2S,5R)-4-benzyl-2,5-diethylpiperazin-1-yl)-4-methyl-2-(tetrahydro-2H-pyran-2-yl)-2,4-dihydro-5H-pyrazolo[4,3-b]pyridin-5-one (4.2 g, 9.1 mmol) and Pd/C (420 mg, 10% in water) in MeOH (50 mL) was shaken under N₂ atmosphere (50 psi) at room temperature for overnight. The reaction mixture was filtered through Celite. The filter cake was washed with EA (50 mL). The combined filtrates were concentrated to dryness to give the titled compound (3.2 g, 94%). MS: M/e 374 (M+1)⁺.

### Intermediate 4: 7-((2S,5R)-2,5-diethyl-4-(1-(quinoxalin-6-yl)ethyl)piperazin-1-yl)-4-methyl-2,4-dihydro-5H-pyrazolo[4,3-b]pyridin-5-one

### Step A: tert-butyl (2S,5R)-2,5-diethyl-4-(1-(quinoxalin-6-yl)ethyl)piperazine-1-carboxylate

To a solution of 1-(quinoxalin-6-yl)ethan-1-ol (1.0 g, 5.75 mmol), tert-butyl (2S,5R)-2,5-diethylpiperazine-1-carboxylate (1.68 g, 6.90 mmol) and (cyanomethyl)trimethylphosphonium iodide (2.1 g, 8.62 mmol) in CH₃CN (12 mL) was added DIPEA (3.71 g, 28.75 mmol). The mixture solution was degassed 3 times under N₂ atmosphere. Then the mixture solution was stirred at 105° C. for 24 hours. The reaction was quenched with saturated NH₄Cl (20 mL) at room temperature. The resulting mixture was extracted with EA (35 mL×2). The combined organic layers were dried over Na₂SO₄ and concentrated under reduced pressure. The resulting residue was purified by flash column chromatography to give the titled compound (1.1 g, 48%). MS: M/e 399 (M+1)⁺.

### Step B: 6-(1-((2R,5S)-2,5-diethylpiperazin-1-yl)ethyl)quinoxaline

To a solution of tert-butyl (2S,5R)-2,5-diethyl-4-(1-(quinoxalin-6-yl)ethyl)piperazine-1-carboxylate (500 mg, 1.26 mmol) in DCM (10 mL) at room temperature was added TFA (2 mL). The reaction mixture was stirred at room temperature for 4 hours, and concentrated under reduced pressure to give the crude product (TFA salt). The crude product was basified by Na₂CO₃ (4M) to pH ˜10 and extracted with EA (35 mL×3). The combined organic layers were washed with brine (20 mL×3), dried over Na₂SO₄ and concentrated under reduced pressure to give the titled compound (300 mg, 80%). ¹H NMR (400 MHz, CDCl₃) δ 8.83 (s, 2H), 8.06 (s, 2H), 7.98-7.69 (m, 1H), 4.50 (s, 1H), 3.10-2.73 (m, 2H), 2.62-2.39 (m, 2H), 2.33-2.04 (m, 3H), 1.95-1.67 (m, 2H), 1.66-1.57 (m, 1H), 1.46-1.34 (m, 2H), 1.32-1.14 (m, 2H), 1.05-0.96 (m, 1.5H), 0.94-0.86 (m, 3H), 0.76-0.71 (m, 1.5H) ppm. MS: M/e 299 (M+1)⁺.

### Step C: 7-((2S,5R)-2,5-diethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of 6-(1-((2R,5S)-2,5-diethylpiperazin-1-yl)ethyl)quinoxaline (286 mg, 0.96 mmol) and Intermediate 2 (305 mg, 0.8 mmol) in CH₃CN (15 mL) was added DIPEA (206 mg, 1.6 mmol). The resulting mixture was heated at 90° C. under N₂ for 96 hours, cooled to room temperature, diluted with water (50 mL), and extracted with EA (60 mL×3). The combined organic layers were washed with brine, dried over Na₂SO₄, filtered, and concentrated, and The resulting residue was purified by flash column chromatography (DCM/MeOH=15/1) to give the titled compound (280 mg, 66%). MS: M/e 530 (M+1)⁺.

### Step D: 7-((2S,5R)-2,5-diethyl-4-(1-(quinoxalin-6-yl)ethyl)piperazin-1-yl)-4-methyl-2,4-dihydro-5H-pyrazolo[4,3-b]pyridin-5-one (Intermediate 4)

To a solution of 7-((2S,5R)-2,5-diethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (280 mg, 0.53 mmol) in DCM (4 mL) was added TFA (4 mL). The resulting mixture was stirred at room temperature overnight. Another portion of TFA (2 mL) was added into the reaction and stirred at room temperature for 4 hours. The reaction mixture was concentrated under reduced pressure, diluted with the mixture of water/DCM, basified with saturated NaHCO₃ solution to pH 7-8 and extracted with DCM (60 mL×2). The combined organic layers were washed with brine, dried over Na₂SO₄, filtered and concentrated, and The resulting residue was purified by flash column chromatography (DCM/MeOH=10/1) to give the titled compound (100 mg, 42%). MS: M/e 446 (M+1)⁺.

### Intermediate 5: 2-(7-((2S,5R)-2,5-diethylpiperazin-1-yl)-4-methyl-5-oxo-4,5-dihydro-2H-pyrazolo[4,3-b]pyridin-2-yl)acetonitrile

### Step A: tert-butyl (2R,5S)-2,5-diethyl-4-(4-methyl-5-oxo-4,5-dihydro-2H-pyrazolo[4,3-b]pyridin-7-yl)piperazine-1-carboxylate

To a stirred solution of Intermediate 3 (3 g, 8.04 mmol) in MeOH (20 mL) was added HCl (5 mL, 4.0 M in 1,4-dioxane). The reaction mixture was stirred over weekend, concentrated to give a residue, which was treated with THF/H₂O (50 mL/20 mL, v/v), K₂CO₃ (3.3 g, 24.1 mmol) was added, followed by Boc₂O (1.75 g, 8.04 mmol). Then the mixture was stirred for 1 hour, acidified to pH=4-5 with citric acid, extracted with EA (30 mL×3). The combined organic layers were washed with brine, dried over Na₂SO₄ and concentrated to dryness. The resulting residue was purified by flash column chromatography to give the titled compound (1.4 g, 45%). MS: M/e 390 (M+1)⁺.

### Step B: tert-butyl (2R,5S)-4-(2-(cyanomethyl)-4-methyl-5-oxo-4,5-dihydro-2H-pyrazolo[4,3-b]pyridin-7-yl)-2,5-diethylpiperazine-1-carboxylate

To a stirred solution of tert-butyl (2R,5S)-2,5-diethyl-4-(4-methyl-5-oxo-4,5-dihydro-2H-pyrazolo[4,3-b]pyridin-7-yl)piperazine-1-carboxylate (1.4 g, 3.6 mmol) in DMF/H₂O (10 mL/2 mL) was added K₂CO₃ (1.49 g, 10.8 mmol), followed by 2-iodoacetonitrile (482.4 mg, 7.2 mmol). The reaction mixture was stirred for overnight, poured into H₂O (30 mL), and extracted with EA (30 mL×3). The combined organic layers were washed with brine, dried over Na₂SO₄ and concentrated to dryness. The resulting residue was purified by flash column chromatography to give the titled compound (1 g, 65%). MS: M/e 429 (M+1)⁺.

### Step C: 2-(7-((2S,5R)-2,5-diethylpiperazin-1-yl)-4-methyl-5-oxo-4,5-dihydro-2H-pyrazolo[4,3-b]pyridin-2-yl)acetonitrile (Intermediate 5)

To a stirred solution of tert-butyl (2R,5S)-4-(2-(cyanomethyl)-4-methyl-5-oxo-4,5-dihydro-2H-pyrazolo[4,3-b]pyridin-7-yl)-2,5-diethylpiperazine-1-carboxylate (1 g, 2.34 mmol) in CH₂Cl₂ (15 mL) was added TFA (3 mL). Then reaction mixture was stirred for 5 hours, concentrated to give a residue, basified to pH=10-11 with saturated NaHCO₃ aq., and extracted with CH₂Cl₂/IPA (3/1, 30 mL×3). The combined organic layers were washed with brine, dried over Na₂SO₄, and concentrated to give the titled compound (752 mg, 98%). ¹H NMR (400 MHz, DMSO-d6) δ 7.94 (s, 1H), 5.59 (s, 2H), 5.35 (s, 1H), 4.31 (s, 1H), 4.08 (d, J=13.2 Hz, 1H), 3.23 (s, 3H), 3.04 (dd, J=12.8, 4.4 Hz, 1H), 2.80-2.72 (m, 1H), 2.65-2.59 (m, 1H), 1.83-1.72 (m, 1H), 1.64-1.41 (m, 3H), 0.87-0.75 (m, 8H) ppm. MS: M/e 329 (M+1)⁺.

### Intermediate 6: 7-((2S,5R)-5-ethyl-2-methyl-4-(1-(quinoxalin-6-yl)ethyl)piperazin-1-yl)-4-methyl-2,4-dihydro-5H-pyrazolo[4,3-b]pyridin-5-one

### Step A: methyl (R)-2-(benzylamino)butanoate

To a solution of methyl (R)-2-aminobutanoate hydrogen chloride (100 g, 651 mmol) and K₂CO₃ (225 g, 1.628 mol) in CH₃CN (700 mL) was added (bromomethyl)benzene (122.5 g, 716 mmol) dropwise at 0° C. The reaction mixture was stirred at room temperature for 12 hours. After filtration, the reaction solution was concentrated under reduced pressure. The resulting residue was purified by flash column chromatography to give the titled compound (70 g, 52%). MS: M/e 208 (M+1)⁺.

### Step B: methyl (R)-2-((S)—N-benzyl-2-((tert-butoxycarbonyl)amino)propanamido)butanoate

To a solution of methyl (R)-2-(benzylamino)butanoate (35 g, 169 mmol), (tert-butoxycarbonyl)-L-alanine (48 g, 254 mmol) and HATU (116 g, 304 mmol) in DCM (400 mL) was added NMM (43 g, 422 mmol). The reaction mixture solution was stirred at room temperature for 24 hours. The reaction was quenched with saturated NaCl (100 mL) at room temperature. The resulting mixture was extracted with DCM (300 mL×2). The combined organic layers were dried over Na₂SO₄ and concentrated under reduced pressure. The resulting residue was purified by flash column chromatography to give the titled compound (45 g, 70%). MS: M/e 379 (M+1)⁺.

### Step C: methyl (R)-2-((S)-2-amino-N-benzylpropanamido)butanoate hydrogen chloride

To a solution of methyl (R)-2-((S)—N-benzyl-2-((tert-butoxycarbonyl)amino)propanamido)butanoate (45 g, 119 mmol) in DCM (250 mL) at room temperature was added HCl (119 mL, 4 M in 1,4-dioxane). The reaction mixture was stirred at room temperature for 4 hours, concentrated under reduced pressure to give the titled compound (32 g, 86%). MS: M/e 279 (M+1)⁺.

### Step D: (3S,6R)-1-benzyl-6-ethyl-3-methylpiperazine-2,5-dione

To a solution of methyl (R)-2-((S)-2-amino-N-benzylpropanamido)butanoate hydrogen chloride (32 g, 102 mmol) in NaHCO₃ (150 mL, 4M) was added EA (150 mL). The reaction mixture was stirred at room temperature for 2 hours. The combined organic layers were dried over Na₂SO₄ and concentrated under reduced pressure to give the titled compound (22 g, 87%). MS: M/e 247 (M+1)⁺.

### Step E: (2R,5S)-1-benzyl-2-ethyl-5-methylpiperazine

To a solution of (3S,6R)-1-benzyl-6-ethyl-3-methylpiperazine-2,5-dione (20 g, 81.3 mmol) in THF (300 mL) at 0° C. was added LiAlH₄ (6.2 g, 163 mmol) in portion. The reaction mixture was stirred at 70° C. for 36 hours and added H₂O (6.2 mL), followed NaOH (6.2 mL, 20%) and H₂O (12.4 mL) at 0° C. to quench the reaction solution. After filtration, the combined organic layers were dried over Na₂SO₄ and concentrated under reduced pressure to give the titled compound (11 g, 61%). MS: M/e 219 (M+1)⁺.

### Step F: tert-butyl (2S,5R)-4-benzyl-5-ethyl-2-methylpiperazine-1-carboxylate

To a solution of (2R,5S)-1-benzyl-2-ethyl-5-methylpiperazine (11 g, 50 mmol) and Boc₂O (12 g, 55 mmol) in DCM (200 mL) was added Et₃N (7.6 g, 75 mmol). The reaction mixture was stirred at room temperature for 12 hours. The reaction was quenched with saturated NaCl (100 mL) at room temperature. The resulting mixture was extracted with DCM (200 mL×2). The combined organic layers were dried over Na₂SO₄ and concentrated under reduced pressure. The resulting residue was purified by flash column chromatography to give the titled compound (14 g, 69%). MS: M/e 319 (M+1)⁺.

### Step G: tert-butyl (2S,5R)-5-ethyl-2-methylpiperazine-1-carboxylate

To a solution of tert-butyl (2S,5R)-4-benzyl-5-ethyl-2-methylpiperazine-1-carboxylate (10 g, 31.4 mmol) and Pd/C (2.5 g, 10% in water) in MeOH (60 mL) at room temperature was added AcOH (2 mL). The resulting mixture was degassed 3 times under H₂ atmosphere, and stirred at room temperature under H₂ atmosphere for 12 hours. After filtration, the combined organic layers were concentrated under reduced pressure to give the crude product (AcOH salt). The crude product was basified by Na₂CO₃ (4M) to pH ˜10 and extracted with EA (80 mL×3). The combined organic layers were washed with brine (20 mL×3), dried over Na₂SO₄ and concentrated under reduced pressure to give the titled compound (5.5 g, 77%). ¹H NMR (400 MHz, CDCl₃) δ 4.21-4.09 (m, 1H), 3.72-3.69 (d, J=12.9 Hz, 1H), 3.24-3.19 (dd, J=13.7, 4.0 Hz, 1H), 3.14-3.10 (dd, J=12.8, 4.7 Hz, 1H), 2.86-2.75 (m, 1H), 2.53-2.49 (dd, J=12.8, 2.7 Hz, 1H), 2.33-2.12 (m, 1H), 1.65-1.54 (m, 2H), 1.46 (s, 9H), 1.25-1.24 (d, J=4.0 Hz, 3H), 0.98-0.91 (m, 3H) ppm. MS: M/e 229 (M+1)⁺.

### Step H: tert-butyl (2S,5R)-5-ethyl-2-methyl-4-(1-(quinoxalin-6-yl)ethyl)piperazine-1-carboxylate

A sealed tube was charged with 1-(quinoxalin-6-yl)ethan-1-ol (5 g, 28.7 mmol), tert-butyl (2S,5R)-5-ethyl-2-methylpiperazine-1-carboxylate (6.5 g, 28.7 mmol), (cyanomethyl)trimethylphosphonium iodide (8.3 g, 34.4 mmol), DIPEA (7.4 g, 57.4 mmol) and acetonitrile (100 ml). The reaction mixture was stirred at 105° C. for overnight, and cooled to room temperature. The solvent was removed. Water was added and the aqueous was extracted with EA. The combined organic layers were washed with brine, dried over Na₂SO₄, and concentrated to dryness. The resulting residue was purified by flash column chromatography (EA/PE) to give the titled compound (10.5 g, crude). MS: M/e 385 (M+1)⁺.

### Step I: 6-(1-((2R,5S)-2-ethyl-5-methylpiperazin-1-yl)ethyl)quinoxaline

To a solution of tert-butyl (2S,5R)-5-ethyl-2-methyl-4-(1-(quinoxalin-6-yl)ethyl)piperazine-1-carboxylate (10.5 g) in DCM (100 mL) was added TFA (30 ml). The reaction mixture was stirred at RT for 4 hours, concentrated to dryness. The resulting residue was dissolved into water. The resulting mixture was extracted with EA. The aqueous layer was adjusted pH to 12-13 with saturated Na₂CO₃ aq. and extracted with DCM (50 mL×2). The combined organic layers were washed with brine, dried over Na₂SO₄, and concentrated to dryness. The resulting residue (4.6 g) was used in the next step without further purification. MS: M/e 285 (M+1)⁺.

### Step G: 7-((2S,5R)-5-ethyl-2-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of 6-(1-((2R,5S)-2-ethyl-5-methylpiperazin-1-yl)ethyl)quinoxaline (568 mg, 2 mmol) in dioxane (5 mL) was added triethylamine hydrochloride, Intermediate 2 (914 mg, 2.4 mmol) and DIPEA (774 mg, 6 mmol). The reaction mixture was stirred at 100° C. for weekend, concentrated to dryness. The reaction mixture was quenched with water. The resulting mixture was extracted with EA (100 mL×2). The combined organic layers were washed with brine, dried over Na₂SO₄, filtered and concentrated to dryness. The resulting residue was purified by flash column chromatography (MeOH/DCM) to give the titled compound (1 g, 95%). ¹H NMR (400 MHz, CD₃OD) δ 8.87 (dd, J=6.0, 4.0 Hz, 2H), 8.16-8.01 (m, 3H), 7.99-7.93 (m, 1H), 5.65-5.52 (m, 2H), 5.49 (s, 1H), 4.09-3.91 (m, 2.5H), 3.75 (dd, J=15.1, 10.3 Hz, 1H), 3.56 (s, 1.5H), 3.44 (s, 3H), 3.25-3.01 (m, 1H), 2.95-2.80 (m, 1H), 2.26-2.12 (m, 2H), 2.07-1.98 (m, 2H), 1.77 (d, J=20.1 Hz, 2H), 1.64 (dd, J=13.3, 5.9 Hz, 3H), 1.48-1.42 (m, 4.5H), 1.22 (d, J=6.5 Hz, 1.5H), 0.91-0.85 (m, 3H) ppm. MS: M/e 516 (M+1)⁺.

### Step K: 7-((2S,5R)-5-ethyl-2-methyl-4-(1-(quinoxalin-6-yl)ethyl)piperazin-1-yl)-4-methyl-2,4-dihydro-5H-pyrazolo[4,3-b]pyridin-5-one (Intermediate 6)

To a solution of 7-((2S,5R)-5-ethyl-2-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (300 mg) in MeOH (4 mL) was added HCl (2 mL, 4 M in dioxane). The reaction mixture was stirred at room temperature for 4 hours and concentrated under reduced pressure. The resulting residue (350 mg, crude) was used to next step directly without further purification. MS: M/e 432 (M+1)⁺.

### Intermediate 7: 4-(4-methoxybenzyl)-5-oxo-2-(tetrahydro-2H-pyran-2-yl)-4,5-dihydro-2H-pyrazolo[4,3-b]pyridin-7-yl trifluoromethanesulfonate

### Step A: ethyl 4-((4-methoxybenzyl)amino)-1-(tetrahydro-2H-pyran-2-yl)-1H-pyrazole-3-carboxylate

A mixture of ethyl 4-amino-1-(tetrahydro-2H-pyran-2-yl)-1H-pyrazole-3-carboxylate (5 g, 20.92 mmol), 4-methoxybenzaldehyde (2.85 g, 20.92 mmol), sodium triacetoxyborohydride (6.65 g, 31.38 mmol) in DCM (100 mL) was stirred at RT for 3 hours. The reaction mixture was extracted with DCM (500 mL).

The organic layer was washed with water, dried and concentrated to dryness. The resulting residue was purified by flash column chromatography (PE:EA=1:1) to give the titled compound (7.23 g, 96%). MS: M/e 360 (M+1)+

### Step B: ethyl 4-(N-(4-methoxybenzyl)acetamido)-1-(tetrahydro-2H-pyran-2-yl)-1H-pyrazole-3-carboxylate

A mixture of ethyl 4-((4-methoxybenzyl)amino)-1-(tetrahydro-2H-pyran-2-yl)-1H-pyrazole-3-carboxylate (1 g, 2.785 mmol), acetic anhydride (5 mL) in pyridine (20 mL) was stirred at RT for overnight. The reaction mixture was concentrated to give the crude product, which was dissolved into water. The resulting solution was extracted with DCM (100 mL×2). The organic layer was dried and concentrated to dryness. The resulting residue was purified by flash column chromatography (PE:EA=1:1) to give the titled compound (1.1 g, 98%). MS: M/e 402 (M+1)+

### Step C: 7-hydroxy-4-(4-methoxybenzyl)-2-(tetrahydro-2H-pyran-2-yl)-2,4-dihydro-5H-pyrazolo[4,3-b]pyridin-5-one

To a solution of ethyl 4-(N-(cyclopropylmethyl)acetamido)-1-(tetrahydro-2H-pyran-2-yl)-1H-pyrazole-3-carboxylate (1.1 g, 2.743 mmol) in THF under N₂ was added L1HMDS (4.1 mL, 4.1 mmol) dropwise at −78° C. and stirred for 2 hours. The reaction mixture was quenched with AcOH/water and extracted with DCM (100 mL×2). The organic layer was dried and concentrated to dryness. The resulting residue was purified by flash column chromatography (DCM:MeOH=10:1) to give the titled compound (800 mg, 82%). MS: M/e 356 (M+1)⁺.

### Step D: 4-(4-methoxybenzyl)-5-oxo-2-(tetrahydro-2H-pyran-2-yl)-4,5-dihydro-2H-pyrazolo[4,3-b]pyridin-7-yl trifluoromethanesulfonate (Intermediate 7)

A mixture of 7-hydroxy-4-(4-methoxybenzyl)-2-(tetrahydro-2H-pyran-2-yl)-2,4-dihydro-5H-pyrazolo[4,3-b]pyridin-5-one (800 mg, 2.253 mmol), 1,1,1-trifluoro-N-phenyl-N-((trifluoromethyl)sulfonyl)methanesulfonamide (1.2 g, 3.380 mmol) and K₂CO₃ (933 mg, 6.761 mmol) in DMF (15 mL) was stirred under N₂ at room temperature for overnight. The reaction mixture was quenched with water and extracted with DCM (50 mL×2). The organic layer was dried and concentrated to dryness. The resulting residue was purified by flash column chromatography (DCM:MeOH=20:1) to give the titled compound (520 mg, 47%). MS: M/e 488 (M+1)⁺.

### Intermediate 8: 4-(3,4-dimethoxybenzyl)-5-oxo-2-(tetrahydro-2H-pyran-2-yl)-4,5-dihydro-2H-pyrazolo[4,3-b]pyridin-7-yl trifluoromethanesulfonate

### Step A: ethyl 4-((3,4-dimethoxybenzyl)amino)-1-(tetrahydro-2H-pyran-2-yl)-1H-pyrazole-3-carboxylate

A mixture of ethyl 4-amino-1-(tetrahydro-2H-pyran-2-yl)-1H-pyrazole-3-carboxylate (5 g, 20.92 mmol), 3,4-dimethoxybenzaldehyde (3.47 g, 20.92 mmol), sodium triacetoxyborohydride (6.65 g, 31.38 mmol) in DCM (100 mL) was stirred at room temperature for 3 hours. The reaction mixture was quenched with water and extracted with DCM (250 mL×2). The organic layer was dried and concentrated to dryness. The resulting residue was purified by flash column chromatography (PE:EA=1:1) to give the titled compound (7.33 g, 90%). MS: M/e 390 (M+1)⁺.

### Step B: ethyl 4-(N-(3,4-dimethoxybenzyl)acetamido)-1-(tetrahydro-2H-pyran-2-yl)-1H-pyrazole-3-carboxylate

A mixture of ethyl 4-((3,4-dimethoxybenzyl)amino)-1-(tetrahydro-2H-pyran-2-yl)-1H-pyrazole-3-carboxylate (1 g, 2.571 mmol), acetic anhydride (5 mL) in pyridine (20 mL) was stirred at RT for overnight. The reaction mixture was concentrated to give crude product which was dissolved into water. The resulting solution was extracted with DCM (100 mL×2). The organic layer was dried and concentrated to dryness. The resulting residue was purified by flash column chromatography (PE:EA=1:1) to give the titled compound (0.95 g, 86%). MS: M/e 432 (M+1)⁺.

### Step C: 4-(3,4-dimethoxybenzyl)-7-hydroxy-2-(tetrahydro-2H-pyran-2-yl)-2,4-dihydro-5H-pyrazolo[4,3-b]pyridin-5-one

To a solution of ethyl 4-(N-(3,4-dimethoxybenzyl)acetamido)-1-(tetrahydro-2H-pyran-2-yl)-1H-pyrazole-3-carboxylate (0.95 g, 2.205 mmol) in THF under N₂ was added L1HMDS (3.3 mL, 3.3 mmol) dropwise at −78° C. and stirred for 2 hours. The reaction mixture was quenched with AcOH. The resulting mixture was extracted with DCM (200 mL) and washed with water. The organic layer was dried and concentrated to dryness. The resulting residue was purified by flash column chromatography (DCM:MeOH=10:1) to give the titled compound (720 mg, 85%). MS: M/e 386 (M+1)⁺.

### Step D: 4-(3,4-dimethoxybenzyl)-5-oxo-2-(tetrahydro-2H-pyran-2-yl)-4,5-dihydro-2H-pyrazolo[4,3-b]pyridin-7-yl trifluoromethanesulfonate (Intermediate 8)

A mixture of 4-(3,4-dimethoxybenzyl)-7-hydroxy-2-(tetrahydro-2H-pyran-2-yl)-2,4-dihydro-5H-pyrazolo[4,3-b]pyridin-5-one (720 mg, 1.87 mmol), 1,1,1-trifluoro-N-phenyl-N-((trifluoromethyl)sulfonyl)methanesulfonamide (1 g, 2.805 mmol), K₂CO₃ (516 mg, 3.740 mmol) in DMF (15 mL) was stirred under N₂ at room temperature for overnight. The reaction mixture was extracted with DCM (100 mL) and washed with water. The organic layer was dried and concentrated to dryness. The resulting residue was purified by flash column chromatography (DCM:MeOH=20:1) to give the titled compound (720 mg, 75%). MS: M/e 518 (M+1)⁺.

### Intermediate 9: 4-methyl-2-(tetrahydro-2H-pyran-2-yl)-2,4-dihydro-5H-pyrazolo[4,3-d]pyrimidine-5,7 (6H)-dione

### Step A: 4-nitro-1-(tetrahydro-2H-pyran-2-yl)-1H-pyrazole-3-carboxamide

To ethyl-4-nitro-1-(tetrahydro-2H-pyran-2-yl)-1H-pyrazole-3-carboxylate (8 g, 29.7 mmol) was added a solution of NH₃ (7M) in MeOH (80 mL). The reaction mixture was stirred at 80° C. for 16 hours, cooled to room temperature and concentrated to dryness. The resulting residue was treated to slurry with PE/EA to give the titled compound (6.8 g, 95%). MS: M/e 241 (M+1)⁺.

### Step B: 4-amino-1-(tetrahydro-2H-pyran-2-yl)-1H-pyrazole-3-carboxamide

To a solution of 4-nitro-1-(tetrahydro-2H-pyran-2-yl)-1H-pyrazole-3-carboxamide (6.2 g, 25.8 mmol) in MeOH (100 mL) was added Pd/C (0.6 g, 10% in water). The resulting mixture was stirred at room temperature under H₂ atmosphere. The reaction mixture was filtered and washed with MeOH. The combined filtrate was concentrated to give the titled compound (5.1 g, 94%). MS: M/e 211 (M+1)⁺.

### Step C: 4-((2,4-dinitrophenyl)sulfonamido)-1-(tetrahydro-2H-pyran-2-yl)-1H-pyrazole-3-carboxamide

To a solution of 4-amino-1-(tetrahydro-2H-pyran-2-yl)-1H-pyrazole-3-carboxamide (4.6 g, 21.9 mmol) and TEA (3.33 g, 33 mmol) in THF (0.2 L) was added 2,4-dinitrobenzenesulfonyl chloride (6.11 g 23 mmol) dropwise. The reaction mixture was stirred at room temperature for 6 hours, and concentrated to dryness.

The resulting residue was diluted with water and extracted with DCM (100 mL×3). The combined organic layers were washed with brine, dried over Na₂SO₄, filtered and concentrated to dryness. The resulting residue was purified by column flash column chromatography to give the titled compound (5.3 g, 55%). MS: M/e 441 (M+1)⁺.

### Step D: 4-((N-methyl-2,4-dinitrophenyl)sulfonamido)-1-(tetrahydro-2H-pyran-2-yl)-1H-pyrazole-3-carboxamide

To a solution of 4-((2,4-dinitrophenyl)sulfonamido)-1-(tetrahydro-2H-pyran-2-yl)-1H-pyrazole-3-carboxamide (5.3 g, 12 mmol) in DMF (60 mL) was added K₂CO₃ (3.3 g, 24 mmol), followed by CH₃I (3.4 g, 24 mmol). The reaction mixture was stirred at RT for 16 hours. The reaction solvent was concentrated to dryness. The resulting residue was diluted with water and extracted with DCM (100 mL×2). The combined organic layers were washed with brine, dried over Na₂SO₄, filtered, and concentrated to dryness. The resulting residue was purified by flash column chromatography to give the titled compound (4.8 g, 88%). MS: M/e 455 (M+1)⁺.

### Step E: 4-(methylamino)-1-(tetrahydro-2H-pyran-2-yl)-1H-pyrazole-3-carboxamide

To a solution of 4-((N-methyl-2,4-dinitrophenyl)sulfonamido)-1-(tetrahydro-2H-pyran-2-yl)-1H-pyrazole-3-carboxamide (4.8 g, 10.57 mmol) and TEA (2.13 g, 21.14 mmol) in DCM (40 mL) was added 2-mercaptoacetic acid (1.36 g, 14.8 mmol). The reaction mixture was stirred for 6 hours. The reaction was quenched with water. The resulting mixture was extracted with DCM:IPA (3:1, 100 mL×5). The combined organic layers were washed with brine, dried over Na₂SO₄, filtered and concentrated to dryness. The resulting residue was purified by flash column chromatography (DCM:MeOH=10:1) to give the titled compound (1.8 g, 75%). MS: M/e 225 (M+1)⁺.

### Step F: 4-methyl-2-(tetrahydro-2H-pyran-2-yl)-2,4-dihydro-5H-pyrazolo[4,3-d]pyrimidine-5,7 (6H)-dione (Intermediate 9)

To a solution of 4-(methylamino)-1-(tetrahydro-2H-pyran-2-yl)-1H-pyrazole-3-carboxamide (680 mg, 3 mmol) in DMF (15 mL) was added NaH (240 mg, 6 mmol) at 0° C. After 1 hour, CDI (972 mg, 6 mmol) was added to the reaction and the resulting mixture was heated to 80° C. for overnight. The reaction mixture was cooled to room temperature, quenched with H₂O and concentrated to dryness. The resulting residue was purified by flash column chromatography (DCM:MeOH=10:1) to give the titled compound (0.6 g, 80%). ¹HNMR (400 MHz, DMSO-d₆) δ 11.19 (s, 1H), 8.15 (s, 1H), 5.55 (dd, J=2.4, 9.2 Hz, 1H), 3.98-3.89 (m, 1H), 3.74-3.63 (m, 1H), 3.27 (s, 3H), 2.15-1.85 (m, 3H), 1.78-1.63 (m, 1H), 1.62-1.50 (m, 2H) ppm. MS: M/e 251 (M+1)⁺.

### Intermediate 10: 2-(7-((2S,5R)-2,5-dimethylpiperazin-1-yl)-4-methyl-5-oxo-4,5-dihydro-2H-pyrazolo[4,3-b]pyridin-2-yl)acetonitrile

### Step A: tert-butyl (2R,5S)-2,5-dimethyl-4-(4-methyl-5-oxo-2-(tetrahydro-2H-pyran-2-yl)-4,5-dihydro-2H-pyrazolo[4,3-b]pyridin-7-yl)piperazine-1-carboxylate (Intermediate 10A)

To a solution of tert-butyl (2R,5S)-2,5-dimethylpiperazine-1-carboxylate (2.56 g, 12 mmol) and Intermediate 2 (3.81 mg, 10 mmol) in CH₃CN (15 mL) was added DIPEA (3.87 g, 30 mmol). Then the mixture was heated at 90° C. under N₂ for 16 hours. The mixture was cooled to room temperature, diluted with water (150 mL), extracted with EA (80 mL×3). The combined organic layers were washed with brine, dried over Na₂SO₄, filtered and concentrated to dryness. The resulting residue was The resulting residue was purified by flash column chromatography (DCM:MeOH=15:1) to give titled compound (4.9 g, crude). MS: M/e 446 (M+1)⁺.

### Step B: 7-((2S,5R)-2,5-dimethylpiperazin-1-yl)-4-methyl-2-(tetrahydro-2H-pyran-2-yl)-2,4-dihydro-5H-pyrazolo[4,3-b]pyridin-5-one (Intermediate 10B)

To a solution of Intermediate 10A (4.9 g, crude) in DCM (20 mL) was added TFA (5 mL). The resulting mixture was stirred at room temperature overnight. The mixture was diluted with water, basified with sat NaHCO₃ solution, extracted with DCM:IPA (3:1, 80 mL×3), dried over Na₂SO₄, filtered, and concentrated to dryness. The resulting residue was purified by flash column chromatography (DCM:MeOH=5:1, 1 mol/L NH₃ in MeOH) to give titled compound (2.9 g, 84% for 2 steps). ¹HNMR (400 MHz, CD₃OD) δ 8.03 (d, J=2.8 Hz, 1H), 5.65 (s, 1H), 5.60-5.32 (m, 1H), 4.85-4.76 (m, 1H), 4.53-4.42 (m, 1H), 4.11-3.96 (m, 1H), 3.82-3.70 (m, 1H), 3.69-3.57 (m, 2H), 3.56-3.49 (m, 1H), 3.46 (s, 3H), 3.04-2.92 (m, 1H), 2.30-2.00 (m, 3H), 1.85-1.60 (m, 3H), 1.42-1.30 (m, 6H) ppm. MS: M/e 346 (M+1)⁺.

### Step C: tert-butyl (2R,5S)-2,5-dimethyl-4-(4-methyl-5-oxo-4,5-dihydro-2H-pyrazolo[4,3-b]pyridin-7-yl)piperazine-1-carboxylate

To a stirred solution of Intermediate 10B (3 g, 8.70 mmol) in MeOH (20 mL) was added dioxane/HCl(g) (4.0 M, 5 mL). After the addition, the reaction was stirred over a weekend. The reaction mixture was concentrated to give the residue, which was treated with THF/H₂O (50 mL/20 mL), K₂CO₃ (3.60 g, 26.08 mmol) was added, followed by Boc₂O (1.90 g, 8.70 mmol). Then the mixture was stirred for 1 hour. The mixture was acidified to pH=4-5 with citric acid, extracted with EtOAc (30 mL×3). The combined organic layers were washed with brine, dried over Na₂SO₄, concentrated. The resulting residue was purified by flash column chromatography to give the titled compound (1.7 g, 54%). MS: M/e 362 (M+1)⁺.

### Step D: tert-butyl (2R,5S)-4-(2-(cyanomethyl)-4-methyl-5-oxo-4,5-dihydro-2H-pyrazolo[4,3-b]pyridin-7-yl)-2,5-dimethylpiperazine-1-carboxylate

To a stirred solution of the compound of Step C (1.7 g, 4.71 mmol) in DMF/H₂O (10 mL/2 mL) was added K₂CO₃ (1.95 g, 14.1 mmol), followed by 2-iodoacetonitrile (1.18 g, 7.06 mmol). After the addition, the reaction mixture was stirred at RT overnight. The reaction mixture was poured into H₂O (30 mL), extracted with EtOAc (30 mL×3). The combined organic layers were washed with brine, dried over Na₂SO₄, concentrated. The resulting residue was purified by flash column chromatography to give the titled compound (1.3 g, 69%). MS: M/e 401 (M+1)⁺.

### Step E: 2-(7-((2S,5R)-2,5-dimethylpiperazin-1-yl)-4-methyl-5-oxo-4,5-dihydro-2H-pyrazolo[4,3-b]pyridin-2-yl)acetonitrile

To a stirred solution of tert-butyl (2R,5S)-4-(2-(cyanomethyl)-4-methyl-5-oxo-4,5-dihydro-2H-pyrazolo[4,3-b]pyridin-7-yl)-2,5-dimethylpiperazine-1-carboxylate (1.3 g, 3.25 mmol) in CH₂Cl₂ (15 mL) was added TFA (3 mL). Then the mixture was stirred for 5 hours. The reaction mixture was concentrated to give the residue, basified to pH=10-11 with aq. NaHCO₃, extracted with CH₂Cl₂/IPA (3/1, 30 mL×3). The combined organic layers were washed with brine, dried over Na₂SO₄ and concentrated to give the titled compound (870 mg, 89%). MS: M/e 301 (M+1)⁺.

### Intermediate 11: 2-(but-2-yn-1-yl)-7-((2S,5R)-5-ethyl-2-methylpiperazin-1-yl)-4-methyl-2,4-dihydro-5H-pyrazolo[4,3-b]pyridin-5-one

### Step A: tert-butyl (2R,5S)-2-ethyl-5-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (Intermediate 11A)

A mixture of Intermediate 2 (19 g, 50 mmol), tert-butyl 2R,5S)-2-ethyl-5-methylpiperazine-1-carboxylate (12.54 g, 55 mmol) and DIPEA (12.9 g, 0.1 mol) in CH₃CN (200 mL) was stirred at 100° C. overnight in a sealed tube. The reaction mixture was concentrated to dryness, The resulting residue was dissolved in CH₂Cl₂ (200 mL) and washed with H₂, brine, dried over Na₂SO₄ and concentrated. The resulting residue was purified by flash column chromatography to give the titled compound (20 g, 83.5%). MS: M/e 460 (M+1)⁺.

### Step B: 7-((2S,5R)-5-ethyl-2-methylpiperazin-1-yl)-4-methyl-2,4-dihydro-5H-pyrazolo[4,3-b]pyridin-5-one

To a stirred solution of Intermediate 11A (6 g, 12.5 mmol) in MeOH (50 mL) was added dioxane/HCl(g) (4.0 M, 50 mL). Then the mixture was stirred for 3 days at 50° C. The reaction mixture was concentrated to give the titled compound (crude), which was used to the next step directly. MS: M/e 276 (M+1)⁺.

### Step C: tert-butyl (2R,5S)-2-ethyl-5-methyl-4-(4-methyl-5-oxo-4,5-dihydro-2H-pyrazolo[4,3-b]pyridin-7-yl)piperazine-1-carboxylate

7-((2S,5R)-5-ethyl-2-methylpiperazin-1-yl)-4-methyl-2,4-dihydro-5H-pyrazolo[4,3-b]pyridin-5-one (12.5 mmol) was dissolved in THF/H₂O (80 mL/80 mL), then basified to pH=7-8 with NaHCO₃. To the reaction mixture was added NaHCO₃ (2.1 g, 25 mmol), followed by Boc₂O (3.28 g, 15 mmol). After the addition, the reaction mixture was stirred for 4 hours. The reaction mixture was extracted with EtOAc (50 mL×2). The combined organic layers were washed with brine, dried over Na₂SO₄ and concentrated. The resulting residue purified by flash column chromatography to give the titled compound (3.8 g, 81%). MS: M/e 376 (M+1)⁺.

### Step D: tert-butyl (2R,5S)-4-(2-(but-2-yn-1-yl)-4-methyl-5-oxo-4,5-dihydro-2H-pyrazolo[4,3-b]pyridin-7-yl)-2-ethyl-5-methylpiperazine-1-carboxylate

To a stirred solution of tert-butyl (2R,5S)-2-ethyl-5-methyl-4-(4-methyl-5-oxo-4,5-dihydro-2H-pyrazolo[4,3-b]pyridin-7-yl)piperazine-1-carboxylate (3.8 g, 10.1 mmol) in DMF/H₂O (30 mL/10 mL) was added K₂CO₃ (2.78 g, 20.2 mmol), followed by 1-bromobut-2-yne (2.02 g, 15.2 mmol). After the addition, the reaction was stirred at RT overnight. The reaction mixture was poured into H₂O (50 mL), then extracted with EtOAc (40 mL×3). The combined organic layers were washed with brine, dried over Na₂SO₄, concentrated. The resulting residue was purified by flash column chromatography to give the titled compound (2.2 g, 51%). MS: M/e 428 (M+1)⁺.

### Step E: 2-(but-2-yn-1-yl)-7-((2S,5R)-5-ethyl-2-methylpiperazin-1-yl)-4-methyl-2,4-dihydro-5H-pyrazolo[4,3-b]pyridin-5-one

To a stirred solution of tert-butyl (2R,5S)-4-(2-(but-2-yn-1-yl)-4-methyl-5-oxo-4,5-dihydro-2H-pyrazolo[4,3-b]pyridin-7-yl)-2-ethyl-5-methylpiperazine-1-carboxylate (2.2 g, 5.15 mmol) in CH₂Cl₂ (50 mL) was added TMSOTf (2.3 g, 10.3 mmol). After then, the mixture was stirred for 2 hours. The mixture was quenched with aq·Na₂CO₃, extracted with CH₂Cl₂/IPA (3/1, 50 mL×4). The combined organic layers were washed with brine, dried over Na₂SO₄ and concentrated to give the titled compound (1.8 g, 99%). MS: M/e 328 (M+1)⁺.

### Intermediate 12: 5-bromo-4-fluoro-2-isopropoxypyridine

To a solution of 5-bromo-4-fluoropyridin-2-ol (764 mg, 4 mmol) in chloroform (15 ml), 2-iodopropane (743 mg, 4.4 mmol) and silver carbonate (1.32 g, 4.8 mmol) were added and the reaction solution was stirred overnight at room temperature. The reaction mixture was concentrated to dryness. The resulting residue was purified by flash column chromatography to give the titled compound (700 mg, 75%). MS: M/e 234 (M+1)⁺.

### Intermediate 13: 1-(5-chloropyridin-2-yl)ethan-1-ol

To a solution of 5-chloropicolinaldehyde (200 mg, 1.42 mmol) in THF (20 mL) was added methylmagnesium bromide (0.9 mL, 1.42 mmol) at −78° C. and stirred for 1 h. The mixture was extracted with DCM (100 mL) and washed with water. The organic layer was dried over Na₂SO₄ and concentrated to dryness. The resulting residue was purified by prep-TLC (DCM:MeOH=20:1) to give the titled compound (150 mg, 67%). MS: M/e 158 (M+1)⁺.

### Intermediate 14: 3-bromo-1-methylisoquinoline

To a mixture of 1,3-dibromoisoquinoline (1.15 g, 4 mmol) and LiCl (185 mg, 4.4 mmol) in DMF (10 mL) was added tetramethylstannane (787 mg, 4.4 mmol) and Pd(PPh₃)₂Cl₂ (280 mg, 0.4 mmol). The reaction solution was stirred overnight at 100° C. The reaction mixture was concentrated to dryness. The resulting residue was purified by flash column chromatography to give the titled compound (630 mg, 71%). MS: M/e 222 (M+1)⁺.

### Intermediate 15: tetrahydro-2H-pyran-4-yl 4-methylbenzenesulfonate

To a solution of tetrahydro-2H-pyran-4-ol (1.02 g, 10 mmol) and TEA (2 g, 20 mmol) in DCM (20 mL) at 0° C. was added TsCl (2.28 g, 12 mmol). The reaction mixture was stirred at 25° C. overnight. The mixture was diluted with H₂O, extracted with DCM (80 mL), washed with brine, dried over Na₂SO₄, filtered, and concentrated to dryness. The resulting residue was purified by flash column chromatography to give the titled compound (1.4 g, 55%). ¹H NMR (400 MHz, CDCl₃) δ 7.80 (d, J=8.4 Hz, 2H), 7.34 (d, J=8.0 Hz, 2H), 4.73-4.65 (m, 1H), 3.92-3.82 (m, 2H), 3.52-3.42 (m, 2H), 2.45 (s, 3H), 1.91-1.69 (m, 4H), ppm.

### Intermediate 16: 7-bromo-3-methylisoquinoline

### Step A: N-(3-bromobenzyl)-1,1-dimethoxypropan-2-amine

A mixture of (3-bromophenyl)methanamine (744 mg, 4 mmol), 1,1-dimethoxypropan-2-one (472 mg, 4 mmol) in DCM (10 mL). Sodium triacetoxyborohydride (1.27 g, 6 mmol) was then added in one portion and the reaction was stirred RT overnight. The reaction mixture was quenched with saturated NaHCO₃ aq. The aqueous layer was then extracted with EtOAc. The combined organic layers were washed with aqueous solution of NaCl. The organic layer was dried over Na₂SO₄, filtered, and concentrated under reduced pressure. The resulting residue (1 g, crude) was used in the next step without purification. MS: M/e 288 (M+1)⁺.

### Step B: 7-bromo-3-methylisoquinoline (Intermediate 16)

Chlorosulfonic acid (3 mL) was added N-(3-bromobenzyl)-1,1-dimethoxypropan-2-amine (1 g, crude) dropwise. The reaction mixture was heated to 100° C. for 20 minutes, then cooled and poured into K₂CO₃ (aq.). The aqueous suspension was extracted with EtOAc. The organic portion was washed with brine, dried (Na₂SO₄), filtered, and concentrated. The resulting residue was purified by flash column chromatography to give the titled compound (260 mg, 34% yield). MS: M/e 222 (M+1)⁺.

### Intermediate 17: (4-bromophenyl)(4,4-difluoropiperidin-1-yl)methanone

A solution of 4-bromobenzoic acid (500 mg, 2.5 mmol), 4,4-difluoropiperidine (333 mg, 2.75 mmol), HATU (1.15 g, 3 mmol) and DIPEA (645 mg, 5 mmol) in CH₂Cl₂ (10 mL) was stirred overnight. The reaction mixture was diluted with CH₂Cl₂ (20 mL), washed with H₂O, brine, dried over Na₂SO₄ and concentrated to dryness. The resulting residue was purified by flash column chromatography to give the titled compound (705 mg, 92.7%). MS: M/e 304/306 (M+1)⁺.

### Intermediate 18: 2-(6-bromopyridin-3-yl)-2-methylpropanenitrile

To a suspension of NaH (60%, 360 mg, 9 mmol) in THF (5 ml) was added a solution of 2-(6-bromopyridin-3-yl)acetonitrile (591 mg, 3.0 mmol) in THF (5 mL) at 0° C. and the resulting reaction mixture stirred at room temperature for 1 hour. At the conclusion of this period, methyl iodide (1.06 g, 7.5 mmol) was added and then stirring continued for an additional 16 hours. After this time, the reaction mixture was quenched by the addition of saturated ammonium chloride solution and then extracted into ethyl acetate. The combined organic portions were concentrated under reduced pressure and the resulting residue was purified by flash column chromatography (EtOAc:PE=0-50% in 25 minutes) to give the titled compound (130 mg, 19%). MS: M/e 225 (M+1)⁺.

### Intermediate 19: 2-bromo-3-fluoro-5-isopropoxypyridine

To a suspension of NaH (60%, 300 mg, 7.5 mmol) in DMF (5 ml) was added a solution of propan-2-ol (270 mg, 4.5 mmol) in DMF (5 mL) at 0° C. and the resulting reaction mixture stirred at room temperature for 1 hour. At the conclusion of this period, 2-bromo-3,5-difluoropyridine (582 mg, 3 mmol) was added and then stirring continued for an additional 16 hours. After this time, the reaction mixture was quenched by the addition of saturated ammonium chloride solution and then extracted into ethyl acetate. The combined organic portions were concentrated under reduced pressure and the resulting residue was purified by flash column chromatography (EtOAc:PE=0-50% in 20 minutes) to give the titled compound (460 mg, 66%). ¹H NMR (400 MHz, CDCl₃) δ 7.95-7.82 (m, 1H), 6.92 (dd, J=9.7, 1.9 Hz, 1H), 4.54 (hept, J=5.9 Hz, 1H), 1.43 (d, J=6.1 Hz, 6H) ppm. MS: M/e 234 (M+1)⁺.

### Intermediate 20:1-(2,2-difluorobenzo[d][1,3]dioxol-5-yl)ethan-1-ol

### Step A: 1-(2,2-difluorobenzo[d][1,3]dioxol-5-yl)ethan-1-one

A mixture of 5-bromo-2,2-difluorobenzo[d][1,3]dioxole (9.5 g, 40.1 mmol), tributyl(1-ethoxyvinyl)stannane (17.5 g, 48.5 mmol) and Pd(PPh₃)₂Cl₂ (1.4 g, 2.0 mmol) in toluene (100 mL) was stirred at 100° C. under N₂ for 16 hrs. The mixture was cooled and a solution of HCl/Dioxane (4M, 30 mL) was added, and stirred for 10 minutes. The resulted mixture was washed with brine (50 mL×2), NaHCO₃ (50 mL), dried over Na₂SO₄, filtered and concentrated to dryness to give the titled compound (12.5 g, crude), which was used for the next step directly without any further purification. ¹H NMR (400 MHz, DMSO-d6) δ 7.95 (s, 1H), 7.91 (d, J=8.4 Hz, 1H), 7.56 (d, J=8.4 Hz, 1H), 2.59 (s, 3H).

### Step B: 1-(2,2-difluorobenzo[d][1,3]dioxol-5-yl)ethan-1-ol (Intermediate 20)

To a solution of 1-(2,2-difluorobenzo[d][1,3]dioxol-5-yl)ethan-1-one (12.5 g, crude) in MeOH (100 mL) was added NaBH₄ (1.2 g, 35.3 mmol) in portions at 0° C. and the mixture was stirred at rt for 1 hour. The mixture was added brine (200 mL), extracted with EtOAc (100 mL×3). The extracts was combined and washed with brine (100 mL×2), dried over Na₂SO₄ and concentrated to dryness. The resulted residue was purified by column chromatography to give the title compound (5.9 g, 73% for 2 steps). ¹H NMR (400 MHz, DMSO-d6) δ 7.36 (s, 1H), 7.32 (d, J=8.4 Hz, 1H), 7.17 (d, J=8.0 Hz, 1H), 5.31 (d, J=4.4 Hz, 1H), 4.81-4.69 (m, 1H), 1.31 (d, J=6.4 Hz, 3H).

### Compound A1: 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one

### Step A: 7-((2S,5R)-2,5-dimethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of Intermediate 2 (1.15 g, 3 mmol) and Intermediate 1 (0.9 g, 3.32 mmol) in CH₃CN (20 mL) was added DIPEA (0.77 g, 6 mmol). The resulting mixture was heated at 90° C. under N₂ for 60 hours and cooled to room temperature. The reaction mixture was diluted with water (50 mL) and extracted with DCM (80 mL×2). The combined organic layers were washed with brine, dried over Na₂SO₄, filtered and concentrated, and The resulting residue was purified by flash column chromatography to give the titled compound (1.34 g, 89%). MS: M/e 502 (M+1)⁺.

### Step B: 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one

To a solution of 7-((2S,5R)-2,5-dimethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (1.34 g, 2.67 mmol) in DCM (10 mL) was added TFA (20 mL). The resulting mixture was stirred at room temperature overnight. Another portion of TFA (10 mL) was added and the reaction was stirred for 6 hours. The reaction mixture was concentrated under reduced pressure, diluted with the mixture of water/DCM, basified with saturated NaHCO₃ aq. To pH 7˜8 and extracted with DCM:IPA (6:1, 60 mL×3). The combined organic layers were washed with brine, dried over Na₂SO₄, filtered and concentrated. The resulting residue was purified by flash column chromatography to give the titled Compound A1 (0.75 g, 67%). ¹HNMR (400 MHz, CD₃OD) δ 8.93-8.82 (m, 2H), 8.20-7.96 (m, 3H), 7.80 (s, 1H), 5.56 (s, 1H), 5.12-4.92 (m, 0.5H), 4.82-4.50 (m, 1H), 4.40-4.25 (m, 0.5H), 4.05-3.90 (m, 0.5H), 3.86-3.77 (m, 0.5H), 3.75-3.62 (m, 1H), 3.52-3.42 (m, 0.5H), 3.46 (s, 3H), 3.02-2.65 (m, 2H), 2.30-2.15 (m, 0.5H), 1.55-1.35 (m, 4.5H), 1.27-1.16 (m, 3H), 1.12-0.96 (m, 1.5H) ppm. MS: M/e 418 (M+1)⁺.

### Compound A2: 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one (417 mg, 1 mmol) and K₂CO₃ (276 mg, 2 mmol) in DMF (10 mL) was added 2-iodoacetonitrile (250 mg, 1.5 mmol). The reaction mixture was stirred at room temperature overnight. The reaction mixture was diluted with water and extracted with EA (80 mL×2). The combined organic layers were washed with brine, dried over Na₂SO₄, filtered and concentrated to dryness. The resulting residue was purified by flash column chromatography to give the titled Compound A2 (350 mg, 76%), which was separated into Compound A2a (60 mg) and Compound A2b (90 mg b chiral Prep-HPLC. The chiral separation conditions are shown below.

| Column | CHIRALPAK IH | |
|---|---|---|
| Column Size | 2 cm × 25 cm, 5 um | |
| Mobile Phase A | MTBE (0.5% 2M NH₃—MeOH) | |
| Mobile Phase B | ETOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A2: ¹HNMR (400 MHz, CD₃OD) δ 8.90-8.84 (m, 2H), 8.15-8.01 (m, 3H), 7.95-7.90 (m, 1H), 5.57 (s, 1H), 5.49-5.44 (m, 2H), 4.96-4.88 (m, 0.5H), 4.65-4.50 (m, 1H), 4.35-4.20 (m, 0.5H), 4.04-3.93 (m, 0.5H), 3.88-3.78 (m, 0.5H), 3.73-3.64 (m, 1H), 3.51-3.43 (m, 0.5H), 3.43 (s, 3H), 3.12-3.03 (m, 0.5H), 2.98-2.80 (m, 1.5H), 2.25-2.16 (m, 0.5H), 1.51-1.38 (m, 4.5H), 1.26-1.17 (m, 3H), 1.06 (d, J=6.4 Hz, 1.5H) ppm. MS: M/e 457 (M+1)⁺.

Compound A2a (the earlier peak, 2-(7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile): ¹HNMR (400 MHz, CD₃OD) δ 8.88 (d, J=2.0 Hz, 1H), 8.87 (d, J=1.6 Hz, 1H), 8.15-8.01 (m, 3H), 7.92 (s, 1H), 5.57 (s, 1H), 5.46 (s, 2H), 5.02-4.86 (m, 1H), 4.36-4.20 (m, 1H), 4.02-3.91 (m, 1H), 3.51-3.42 (m, 1H), 3.43 (s, 3H), 3.12-3.04 (m, 1H), 2.98-2.85 (m, 2H), 1.44 (t, J=5.6 Hz, 6H), 1.06 (d, J=6.4 Hz, 3H) ppm. MS: M/e 457 (M+1)⁺.

Compound A2b (the later peak, 2-(7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-b]pyridin-2-yl)acetonitrile): ¹HNMR (400 MHz, CD₃OD) δ 8.87 (d, J=2.0 Hz, 1H), 8.86 (d, J=1.6 Hz, 1H), 8.14-8.01 (m, 3H), 7.93 (s, 1H), 5.57 (s, 1H), 5.47 (s, 2H), 4.75-4.46 (m, 2H), 3.90-3.78 (m, 1H), 3.74-3.64 (m, 2H), 3.44 (s, 3H), 2.91-2.82 (m, 1H), 2.26-2.18 (m, 1H), 1.47 (d, J=6.8 Hz, 3H), 1.17-1.18 (m, 6H) ppm. MS: M/e 457 (M+1)⁺.

### Compound A16: 1-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)cyclopropane-1-carbonitrile

To a solution of 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile (23 mg, 0.05 mmol) and NaH (8 mg, 0.2 mmol) in DMSO (1 mL) was added 1,2-dibromoethane (28 mg, 0.15 mmol). The reaction mixture was stirred at room temperature overnight. The reaction mixture was diluted with water, extracted with EA (20 mL×2). The combined organic layers were washed with brine, dried over Na₂SO₄, filtered, and concentrated to dryness. The resulting residue was purified by Prep-TLC (DCM:MeOH=15:1) and further purified by Prep-HPLC to give the titled Compound A16 (3 mg, 12%). ¹HNMR (400 MHz, CD₃OD) δ 8.90-8.80 (m, 2H), 8.00-7.92 (m, 4H), 5.59 (s, 1H), 4.75-4.23 (m, 2H), 4.09-3.64 (m, 2H), 3.51-3.44 (m, 0.5H), 3.45 (s, 3H), 3.19-2.76 (m, 2H), 2.28-2.16 (m, 0.5H), 2.05-1.88 (m, 4H), 1.72-1.05 (m, 9H) ppm. MS: M/e 483 (M+1)⁺

### Compound A17: 2-cyclopropyl-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one

To a solution of 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one (13 mg, 0.03 mmol), cyclopropylboronic acid (3 mg, 0.045 mmol) and Na₂CO₃ (6 mg, 0.06 mmol) in toluene (2 mL) was added Cu(OAc)₂ (6 mg, 0.03 mmol) and 2,2′-bipyridine (4 mg, 0.03 mmol). The reaction mixture was stirred at 80° C. overnight under O₂. The reaction mixture was diluted with water, extracted with EA (25 mL×2). The combined organic layers were washed with brine, dried over Na₂SO₄, filtered and concentrated to dryness. The resulting residue was purified by Prep-TLC (DCM/MeOH=20/1) to give the titled Compound A17 (3 mg, 21%). ¹HNMR (400 MHz, CD₃OD) δ 8.90-8.84 (m, 2H), 8.15-8.05 (m, 3H), 7.88 (s, 1H), 5.52 (s, 1H), 4.77-4.35 (m, 1.5H), 4.00-3.79 (m, 2H), 3.72-3.60 (m, 1.5H), 3.51-3.42 (m, 0.5H), 3.42 (s, 3H), 3.16-2.79 (m, 2H), 2.26-2.14 (m, 0.5H), 1.55-1.40 (m, 4H), 1.24-1.15 (m, 6H), 1.13-1.02 (m, 3H) ppm. MS: M/e 458 (M+1)⁺.

### Compound A19: 2-(3-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b] pyridin-2-yl)-1-(ethylsulfonyl)azetidin-3-yl)acetonitrile

To a solution of 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one (13 mg, 0.03 mmol) in CH₃CN (1 mL) was added 2-(1-(ethylsulfonyl)azetidin-3-ylidene)acetonitrile (8 mg, 0.045 mmol) and DBU (9 mg, 0.06 mmol). The reaction mixture was stirred at room temperature overnight. The reaction mixture was diluted with water and extracted with EA (20 mL×2). The combined organic layers were washed with brine, dried over Na₂SO₄, filtered, and concentrated to dryness. The resulting residue was purified by Prep-TLC (DCM/MeOH=10/1) to give the titled Compound 19 (6 mg, 33%). ¹HNMR (400 MHz, CD₃OD) δ 8.90-8.84 (m, 2H), 8.19-8.16 (m, 1H), 8.14-8.01 (m, 3H), 5.58 (s, 1H), 4.90-4.87 (m, 0.5H), 4.75-4.50 (m, 3H), 4.45-4.32 (m, 0.5H), 4.29-4.20 (m, 2H), 4.02-3.92 (m, 0.5H), 3.86-3.78 (m, 0.5H), 3.73-3.64 (m, 1H), 3.61-3.54 (m, 2H), 3.50-3.44 (m, 0.5H), 3.46 (s, 3H), 3.18-3.04 (m, 2.5H), 2.97-2.80 (m, 1.5H), 2.24-2.17 (m, 0.5H), 1.51-1.37 (m, 4.5H), 1.36-1.27 (m, 3H), 1.26-1.18 (m, 3H), 1.09-1.03 (m, 1.5H) ppm. MS: M/e 604 (M+1)⁺.

### Compound A20: 3-cyclopentyl-3-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)propanenitrile

To a solution of 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one (13 mg, 0.03 mmol) in CH₃CN (1 mL) was added 3-cyclopentylacrylonitrile (8 mg, 0.045 mmol) and DBU (9 mg, 0.06 mmol). The reaction mixture was stirred at room temperature overnight. The reaction mixture was diluted with water and extracted with EA (20 mL×2). The combined organic layers were washed with brine, dried over Na₂SO₄, filtered, and concentrated to dryness. The resulting residue was purified by Prep-TLC (DCM/MeOH=10/1) to give the titled Compound A20 (6 mg, 33%). ¹HNMR (400 MHz, CD₃OD) δ 8.90-8.84 (m, 2H), 8.16-7.98 (m, 3H), 7.94 (t, J=3.2 Hz, 1H), 5.58-5.52 (m, 1H), 4.75-4.32 (m, 2H), 4.02-3.92 (m, 0.5H), 3.88-3.76 (m, 0.5H), 3.74-3.60 (m, 1H), 3.50-3.45 (m, 0.5H), 3.45 (s, 3H), 3.25-3.00 (m, 2.5H), 2.97-2.80 (m, 1.5H), 2.68-2.42 (m, 1H), 2.26-2.17 (m, 0.5H), 1.96-1.83 (m, 1H), 1.78-1.50 (m, 4H), 1.49-1.41 (m, 4.5H), 1.40-1.17 (m, 7H), 1.09-1.01 (m, 1.5H) ppm. MS: M/e 539 (M+1)⁺.

### Compound A22: 2-(but-2-yn-1-yl)-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one

To a solution of 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one (252 mg, 0.6 mmol) and K₂CO₃ (166 mg, 1.2 mmol) in DMF (5 mL) was added 1-bromobut-2-yne (120 mg, 0.9 mmol). The reaction mixture was stirred at room temperature overnight. The reaction mixture was diluted with water and extracted with EA (60 mL×2). The combined organic layers were washed with brine, dried over Na₂SO₄, filtered and concentrated to dryness. The resulting residue was purified by Prep-TLC (DCM:MeOH=15:1) to give the titled Compound A22 (140 mg, 50%), which was further separated by chiral-prep-HPLC to yield Compound A22a (50 mg) and Compound A22b (50 mg). The chiral separation conditions are shown below.

| Column | CHIRALPAK IG-3 | |
|---|---|---|
| Column Size | 2 cm × 25 cm, 5 um | |
| Mobile Phase A | Hex:DCM = 5:1(0.5% 2M NH3—MEOH) | |
| Mobile Phase B | IPA | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A22a (the earlier isomer, 2-(but-2-yn-1-yl)-7-((2S,5R)-2,5-dimethyl-4-((R)-1-(quinoxalin-6-yl)ethyl)piperazin-1-yl)-4-methyl-2,4-dihydro-5H-pyrazolo[4,3-b]pyridin-5-one): ¹HNMR (400 MHz, CD₃OD) δ 8.91-8.80 (m, 2H), 8.15-7.98 (m, 3H), 7.90 (s, 1H), 5.53 (s, 1H), 5.04 (s, 2H), 4.80-4.45 (m, 2H), 3.86-3.74 (m, 1H), 3.71-3.59 (m, 2H), 3.44 (s, 3H), 2.86-2.74 (m, 1H), 2.22-2.14 (m, 1H), 1.86 (s, 3H), 1.52-1.38 (m, 3H), 1.24-1.12 (m, 6H) ppm. MS: M/e 470 (M+1)⁺.

Compound A22b (the later isomer, 2-(but-2-yn-1-yl)-7-((2S,5R)-2,5-dimethyl-4-((S)-1-(quinoxalin-6-yl)ethyl)piperazin-1-yl)-4-methyl-2,4-dihydro-5H-pyrazolo[4,3-b]pyridin-5-one): ¹HNMR (400 MHz, CD₃OD) δ 8.91-8.80 (m, 2H), 8.16-8.00 (m, 3H), 7.90 (s, 1H), 5.53 (s, 1H), 5.03 (s, 2H), 5.02-4.86 (m, 1H), 4.40-4.15 (m, 1H), 4.01-3.91 (m, 1H), 3.51-3.38 (m, 4H), 3.12-3.04 (m, 1H), 2.95-2.81 (m, 2H), 1.85 (s, 3H), 1.48-1.36 (m, 6H), 1.04 (d, J=4.8 Hz, 3H) ppm. MS: M/e 470 (M+1)⁺.

### Compound A23: 2-(but-2-yn-1-yl)-7-((2S,5R)-2,5-diethyl-4-(1-(quinoxalin-6-yl)ethyl)piperazin-1-yl)-4-methyl-2,4-dihydro-5H-pyrazolo[4,3-b]pyridin-5-one

To a solution of Intermediate 4 (14 mg, 0.03 mmol) and K₂CO₃ (8 mg, 0.06 mmol) in DMF (5 mL) was added 1-bromobut-2-yne (6 mg, 0.045 mmol). The reaction mixture was stirred at room temperature overnight. The reaction mixture was diluted with water, extracted with EA (20 mL×2). The combined organic layers were washed with brine, dried over Na₂SO₄, filtered and concentrated to dryness. The resulting residue was purified by Prep-TLC (DCM:MeOH=15:1) to give the titled Compound A23 (2 mg, 13%). ¹HNMR (400 MHz, CD₃OD) δ 8.91-8.86 (m, 2H), 8.16-7.95 (m, 3H), 7.89 (s, 1H), 5.52 (s, 1H), 5.04 (s, 2H), 4.11-4.00 (m, 0.5H), 3.95-3.84 (m, 0.5H), 3.58-3.49 (m, 0.5H), 3.44 (s, 3H), 3.31-3.28 (2H), 3.24-3.14 (m, 0.5H), 3.12-3.02 (m, 0.5H), 2.98-2.87 (m, 0.5H), 2.77-2.62 (m, 1H), 2.46-2.29 (m, 1H), 2.21-2.10 (m, 0.5H), 2.06-1.91 (m, 0.5H), 1.84 (s, 3H), 1.76-1.50 (m, 3H), 1.50-1.36 (m, 3H), 1.10-0.90 (m, 3H), 0.72-0.49 (m, 3H) ppm. MS: M/e 498 (M+1)⁺.

### Compound A24: 7-((2S,5R)-2,5-dimethyl-4-(1-(quinoxalin-6-yl)ethyl)piperazin-1-yl)-4-methyl-2-(prop-1-en-2-yl)-2,4-dihydro-5H-pyrazolo[4,3-b]pyridin-5-one

To a solution of 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one (13 mg, 0.03 mmol), 4,4,5,5-tetramethyl-2-(prop-1-en-2-yl)-1,3,2-dioxaborolane (10 mg, 0.06 mmol) and Na₂CO₃ (6 mg, 0.06 mmol) in toluene (2 mL) was added Cu(OAc)₂ (6 mg, 0.03 mmol) and 2,2′-bipyridine (4 mg, 0.03 mmol). The reaction mixture was stirred at 80° C. overnight under O₂. The reaction mixture was diluted with water, extracted with EA (25 mL×2). The combined organic layers were washed with brine, dried over Na₂SO₄, filtered, and concentrated to dryness. The resulting residue was purified by Prep-TLC (DCM/MeOH=20/1) to give the titled Compound A24 (1 mg, 7%). ¹HNMR (400 MHz, CD₃OD) δ 8.95-8.80 (m, 2H), 8.35-7.95 (m, 4H), 5.65-5.45 (m, 2H), 4.75-4.23 (m, 2H), 4.03-3.62 (m, 2H), 3.54-3.40 (m, 4.5H), 3.17-2.73 (m, 2H), 2.50-2.16 (m, 3.5H), 1.55-1.39 (m, 4H), 1.55-1.39 (m, 4H), 1.14-1.02 (m, 1H) ppm. MS: M/e 458 (M+1)⁺.

### Compound A25: 7-((2S,5R)-2,5-diethyl-4-(1-(quinoxalin-6-yl)ethyl)piperazin-1-yl)-4-methyl-2-(prop-1-en-2-yl)-2,4-dihydro-5H-pyrazolo[4,3-b]pyridin-5-one

To a solution of Intermediate 4 (14 mg, 0.03 mmol), 4,4,5,5-tetramethyl-2-(prop-1-en-2-yl)-1,3,2-dioxaborolane (10 mg, 0.06 mmol) and Na₂CO₃ (6 mg, 0.06 mmol) in toluene (3 mL) was added Cu(OAc)₂ (5 mg, 0.03 mmol) and 2,2′-bipyridine (5 mg, 0.03 mmol). The reaction mixture was stirred at 80° C. overnight under O₂. The reaction mixture was diluted with water and extracted with EA (60 mL×2). The combined organic layers were washed with brine, dried over Na₂SO₄, filtered, and concentrated to dryness. The resulting residue was purified by Prep-TLC (DCM:MeOH=15:1) to give the titled Compound A25 (1 mg, 6%). ¹HNMR (400 MHz, CD₃OD) δ 8.91-8.84 (m, 2H), 8.19 (s, 1H), 8.16-8.00 (m, 3H), 5.58-5.47 (m, 2H), 4.61 (s, 1H), 4.12-4.02 (m, 0.5H), 3.95-3.82 (m, 0.5H), 3.62-3.52 (m, 0.5H), 3.45 (s, 3H), 3.31-3.28 (2H), 3.26-3.20 (m, 0.5H), 3.16-3.04 (m, 0.5H), 2.98-2.86 (m, 0.5H), 2.80-2.69 (m, 1H), 2.46-2.29 (m, 4H), 2.26-2.14 (m, 0.5H), 2.08-1.94 (m, 0.5H), 1.80-1.55 (m, 3H), 1.49-1.40 (m, 3H), 1.10-0.94 (m, 3H), 0.71-0.49 (m, 3H) ppm. MS: M/e 486 (M+1)⁺.

### Compound A26: 7-((2S,5R)-2,5-dimethyl-4-(4-(trifluoromethoxy)benzyl)piperazin-1-yl)-4-methyl-2-(prop-1-en-2-yl)-2,4-dihydro-5H-pyrazolo[4,3-b]pyridin-5-one

### Step A: tert-butyl(2S,5R)-2,5-dimethyl-4-(4-(trifluoromethoxy)benzyl)piperazine-1-carboxylate

To a solution of tert-butyl (2S,5R)-2,5-dimethylpiperazine-1-carboxylate (214 mg, 1 mmol), (4-(trifluoromethoxy)phenyl)methanol (576 mg, 3 mmol) and (cyanomethyl)trimethylphosphonium iodide (729 mg, 3 mmol) in CH₃CN (10 mL) was added DIPEA (774 mg, 6 mmol). The reaction mixture was sealed in a bottle and heated at 90° C. for 16 hours, and then cooled to room temperature, diluted with water, extracted with EA (60 mL×2). The combined organic layers were washed with brine, dried over Na₂SO₄, filtered, and concentrated to dryness. The resulting residue was purified by flash column chromatography (EA:PE=1:4) to give the titled compound (380 mg, 94%). ¹H NMR (400 MHz, CD₃OD) δ 7.38 (d, J=7.2 Hz, 2H), 7.16 (d, J=6.4 Hz, 2H), 4.28-4.16 (m, 1H), 3.72-3.56 (m, 2H), 3.50-3.40 (m, 1H), 3.36-3.24 (m, 1H), 3.05-2.85 (m, 1H), 2.78-2.64 (m, 1H), 2.20-2.12 (m, 1H), 1.46 (s, 9H), 1.28-1.18 (m, 3H), 1.04-0.92 (m, 3H) ppm. MS: M/e 389 (M+1)⁺.

### Step B: (2R,5S)-2,5-dimethyl-1-(4-(trifluoromethoxy)benzyl)piperazine

To a solution of tert-butyl(2S,5R)-2,5-dimethyl-4-(4-(trifluoromethoxy)benzyl)piperazine-1-carboxylate (380 mg 1 mmol) in DCM (10 mL) was added TFA (4 mL). The reaction mixture was stirred at room temperature overnight, and concentrated to dryness. The resulting residue was dissolved into saturated NaHCO₃ solution and extracted with DCM (60 mL×2). The combined organic layers were washed with brine, dried over Na₂SO₄, filtered and concentrated to dryness. The resulting residue was purified by flash column chromatography (EA:PE=1:1) to give the titled compound (200 mg, 70%). MS: M/e 289 (M+1)⁺.

### Step C: 7-((2S,5R)-2,5-dimethyl-4-(4-(trifluoromethoxy)benzyl)piperazin-1-yl)-4-methyl-2-(tetrahydro-2H-pyran-2-yl)-2,4-dihydro-5H-pyrazolo[4,3-b]pyridin-5-one

To a solution of (2R,5S)-2,5-dimethyl-1-(4-(trifluoromethoxy)benzyl)piperazine (86 mg, 0.3 mmol) and Intermediate 2 (76 mg, 0.2 mmol) in CH₃CN (6 mL) was added DIPEA (78 mg, 0.6 mmol). Then the mixture was heated at 90° C. under N₂ for 60 hours. The reaction mixture was cooled to room temperature, diluted with water (50 mL), extracted with EA (80 mL). The combined organic layers were washed with brine, dried over Na₂SO₄, filtered and concentrated to dryness. The resulting residue was purified by Prep-TLC (DCM:MeOH=15:1) to give titled compound (50 mg, 32%). MS: M/e 520 (M+1)⁺.

### Step D: 7-((2S,5R)-2,5-dimethyl-4-(4-(trifluoromethoxy)benzyl)piperazin-1-yl)-4-methyl-2,4-dihydro-5H-pyrazolo[4,3-b]pyridin-5-one

To a solution of 7-((2S,5R)-2,5-dimethyl-4-(4-(trifluoromethoxy)benzyl)piperazin-1-yl)-4-methyl-2-(tetrahydro-2H-pyran-2-yl)-2,4-dihydro-5H-pyrazolo[4,3-b]pyridin-5-one (50 mg, 0.096 mmol) in DCM (4 mL) was added TFA (4 mL). The resulting mixture was stirred at room temperature overnight. The reaction mixture was concentrated under reduced pressure, diluted with water/DCM, basified with saturated NaHCO₃ solution to pH 7-8, extracted with DCM (60 mL×2). The combined organic layers were washed with brine, dried over Na₂SO₄, filtered and concentrated to dryness. The resulting residue was purified by Prep-TLC (DCM:MeOH=10:1) to give the titled compound (22 mg, 50%). MS: M/e 436 (M+1)⁺.

### Step E: 7-((2S,5R)-2,5-dimethyl-4-(4-(trifluoromethoxy)benzyl)piperazin-1-yl)-4-methyl-2-(prop-1-en-2-yl)-2,4-dihydro-5H-pyrazolo[4,3-b]pyridin-5-one

To a solution of 7-((2S,5R)-2,5-dimethyl-4-(4-(trifluoromethoxy)benzyl)piperazin-1-yl)-4-methyl-2,4-dihydro-5H-pyrazolo[4,3-b]pyridin-5-one (22 mg, 0.046 mmol), 4,4,5,5-tetramethyl-2-(prop-1-en-2-yl)-1,3,2-dioxaborolane (16 mg, 0.092 mmol) and Na₂CO₃ (10 mg, 0.092 mmol) in toluene (3 mL) was added Cu(OAc)₂ (8 mg, 0.046 mmol) and 2,2′-bipyridine (7 mg, 0.046 mmol). The reaction was stirred at 80° C. overnight under O₂ (balloon). The reaction mixture was diluted with water, extracted with EA (50 mL), washed with brine, dried over Na₂SO₄, filtered, and concentrated to dryness. The resulting residue was purified by Prep-TLC (DCM:MeOH=15:1) to give titled Compound A26 (3 mg, 13%). ¹HNMR (400 MHz, CD₃OD) δ 8.18 (s, 1H), 7.50 (d, J=7.6 Hz, 2H), 7.23 (d, J=6.4 Hz, 2H), 5.60-5.50 (m, 2H), 4.92 (s, 1H), 4.76-4.55 (m, 2H), 3.76-3.66 (m, 1H), 3.65-2.54 (m, 2H), 3.45 (s, 3H), 3.02-3.08 (m, 1H), 2.99-2.90 (m, 1H), 2.41-2.37 (m, 1H), 2.35 (s, 3H), 1.33 (d, J=3.6 Hz, 3H), 1.14 (d, J=3.2 Hz, 3H) ppm. MS: M/e 476 (M+1)⁺.

### Compound A28: 2-(7-((2S,5R)-2,5-diethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of Intermediate 4 (90 mg, 0.2 mmol) and K₂CO₃ (55 mg, 0.4 mmol) in DMF (5 mL) was added 2-iodoacetonitrile (66 mg, 0.3 mmol). The reaction was stirred at room temperature overnight. The reaction mixture was diluted with water, extracted with EA (60 mL×2), washed with brine, dried over Na₂SO₄, filtered, and concentrated to dryness. The resulting residue was purified by Prep-TLC (DCM:MeOH=15:1) to give the titled Compound A28 (90 mg) as a mixture of diastereomers, which was further separated by chiral-prep-HPLC to yield Compound A28a (26 mg) and Compound A28b (20 mg). The chiral separation conditions are shown below.

| Column | CHIRAL Cellulose-SB | |
|---|---|---|
| Column Size | 2 cm × 25 cm, 5 um | |
| Mobile Phase A | MtBE(0.5% 2M NH3—MEOH) | |
| Mobile Phase B | MeOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A28a (the earlier peak): ¹HNMR (400 MHz, CD₃OD) δ 8.93-8.84 (m, 2H), 8.12-8.01 (m, 3H), 7.92 (s, 1H), 5.56 (s, 1H), 5.45 (s, 2H), 4.12-4.00 (m, 1H), 3.43 (s, 3H), 3.39-3.26 (m, 3H), 3.14-3.04 (m, 1H), 3.00-2.89 (m, 1H), 2.48-2.36 (m, 1H), 2.26-2.10 (m, 1H), 1.96-1.79 (m, 1H), 1.70-1.54 (m, 2H), 1.51-1.42 (m, 3H), 1.06-0.85 (m, 3H), 0.74-0.65 (m, 3H) ppm. MS: M/e 485 (M+1)⁺.

Compound A28b (the later peak): ¹HNMR (400 MHz, CD₃OD) δ 8.92-8.84 (m, 2H), 8.15-7.98 (m, 3H), 7.93 (s, 1H), 5.55 (s, 1H), 5.47 (s, 2H), 3.96-3.85 (m, 1H), 3.62-3.50 (m, 1H), 3.43 (s, 3H), 3.35-3.28 (m, 2H), 3.27-3.18 (m, 1H), 2.80-2.68 (m, 1H), 2.38-2.30 (m, 1H), 2.06-1.90 (m, 1H), 1.80-1.55 (m, 3H), 1.48-1.37 (m, 3H), 1.12-1.00 (m, 3H), 0.65-0.48 (m, 3H) ppm. MS: M/e 485 (M+1)⁺.

Compound A29: 2-(7-((2S,5R)-4-(1-(4-fluoro-2-(methoxymethyl)phenyl)ethyl)-2,5-dimethylpiperazin-1-yl)-4-methyl-5-oxo-4,5-dihydro-2H-pyrazolo[4,3-b]pyridin-2-yl)acetonitrile

### Step A: tert-butyl (2S,5R)-4-(1-(4-fluoro-2-(methoxymethyl)phenyl)ethyl)-2,5-dimethylpiperazine-1-carboxylate

To a solution of 1-(4-fluoro-2-(methoxymethyl)phenyl)ethan-1-ol (600 mg, 3.26 mmol), tert-butyl tert-butyl (2S,5R)-2,5-dimethylpiperazine-1-carboxylate (1050 mg, 4.9 mmol) and (cyanomethyl)trimethylphosphonium iodide (1584 mg, 6.52 mmol) in CH₃CN (4 mL) was added DIPEA (2103 mg, 16.3 mmol). The mixture solution was degassed 3 times under N₂ atmosphere. Then the mixture solution was stirred at 105° C. for 24 hours. The reaction mixture was quenched with saturated NH₄Cl (20 mL) at room temperature and extracted with EA (35 mL×2). The combined organic layer was dried over Na₂SO₄ and concentrated under reduced pressure. The resulting residue was purified by column flash column chromatography to give the titled compound (800 mg, 65%). MS: M/e 381 (M+1)⁺.

### Step B: (2R,5S)-1-(1-(4-fluoro-2-(methoxymethyl)phenyl)ethyl)-2,5-dimethylpiperazine

To a solution of tert-butyl (2S,5R)-4-(1-(4-fluoro-2-(methoxymethyl)phenyl)ethyl)-2,5-dimethylpiperazine-1-carboxylate (800 mg, 2.105 mmol) in DCM (25 mL) at room temperature was added HCl (6 mL, 4 M in 1,4-dioxane). The mixture was stirred at room temperature for 2 hours. Then the reaction mixture was concentrated under reduced pressure to give the crude product (HCl salt). The crude product was basified by Na₂CO₃ (4M) to pH ˜10 and extracted with EA (35 mL×3). The combined organic layer was washed with brine (20 mL×3), dried over Na₂SO₄ and concentrated under reduced pressure to give the titled compound (400 mg, 68%) which was used directly for next step without purification. MS: M/e 281 (M+1)⁺.

### Step C: 7-((2S,5R)-4-(1-(4-fluoro-2-(methoxymethyl)phenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

To a solution of (2R,5S)-1-(1-(4-fluoro-2-(methoxymethyl)phenyl)ethyl)-2,5-dimethylpiperazine (400 mg, 1.428 mmol) and Intermediate 2 (597 mg, 1.57 mmol) in CH₃CN (5 mL) at room temperature was added DIPEA (921 mg, 7.14 mmol). The mixture was stirred at 105° C. for 24 hours. Then the reaction mixture was concentrated under reduced pressure to give the crude product. The crude product was purified by flash column chromatography to give the titled compound (450 mg, 74%). MS: M/e 512 (M+1)⁺.

### Step D: 7-((2S,5R)-4-(1-(4-fluoro-2-(methoxymethyl)phenyl)ethyl)-2,5-dimethylpiperazin-1-yl)-4-methyl-2,4-dihydro-5H-pyrazolo[4,3-b]pyridin-5-one

To a solution of 7-((2S,5R)-4-(1-(4-fluoro-2-(methoxymethyl)phenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (450 mg, 1.054 mmol) in MeOH (15 mL) at room temperature was added HCl (6 mL, 4 M in 1,4-dioxane). The mixture was stirred at room temperature for 2 hours. Then the reaction mixture was concentrated under reduced pressure to give the crude product (HCl salt). The crude product was basified by Na₂CO₃ (4M) to pH ˜10 and extracted with EA (40 mL×3). The combined organic layer was washed with brine (20 mL×3), dried over Na₂SO₄ and concentrated under reduced pressure to give the titled compound (380 mg, 84%). MS: M/e 428 (M+1)⁺.

### Step E: 2-(7-((2S,5R)-4-(1-(4-fluoro-2-(methoxymethyl)phenyl)ethyl)-2,5-dimethylpiperazin-1-yl)-4-methyl-5-oxo-4,5-dihydro-2H-pyrazolo[4,3-b]pyridin-2-yl)acetonitrile

To a solution of 7-((2S,5R)-4-(1-(4-fluoro-2-(methoxymethyl)phenyl)ethyl)-2,5-dimethylpiperazin-1-yl)-4-methyl-2,4-dihydro-5H-pyrazolo[4,3-b]pyridin-5-one (380 mg, 0.889 mmol) and K₂CO₃ (246 mg, 1.779 mmol) in DMF (10 mL) at room temperature was added 2-iodoacetonitrile (223 mg, 1.334 mmol). The mixture solution was stirred at room temperature for 12 hours. Then the reaction mixture was quenched with saturated NaCl (20 mL) at room temperature and extracted with EA (45 mL×2). The combined organic layer was dried over Na₂SO₄ and concentrated under reduced pressure. The crude product was purified by Prep-TLC (DCM:MeOH=20:1) to give the titled compound Compound A29 (200 mg), which was further separated into Compound A29a (86 mg) and Compound A29b (88 mg) by Prep-HPLC (Method A).

Compound A29: ¹H NMR (400 MHz, DMSO-d6) δ 7.99 (s, 1H), 7.57 (d, J=7.2 Hz, 1H), 7.14 (dd, J=16.3, 9.8 Hz, 2H), 5.62 (d, J=3.3 Hz, 2H), 5.41 (d, J=5.5 Hz, 1H), 4.58 (s, 1H), 4.50 (s, 1H), 3.93-3.64 (m, 1H), 3.28 (s, 3H), 2.93-2.61 (m, 7H), 1.30-1.16 (m, 6H), 1.12-0.97 (m, 3H), 0.90 (d, J=6.6 Hz, 2H). MS: M/e 467 (M+1)⁺

Compound A29a (the earlier peak): ¹H NMR (400 MHz, CD₃OD) δ 7.93 (s, 1H), 7.69 (s, 1H), 7.08-7.05 (m, 2H), 5.56 (s, 1H), 5.48 (s, 2H), 4.61-4.56 (m, 1H), 4.53 (s, 3H), 3.86 (s, 1H), 3.63-3.59 (m, 2H), 3.43 (s, 3H), 3.39 (s, 3H), 2.82-2.79 (d, J=12 Hz, 1H), 2.17-2.14 (d, J=12 Hz, 1H), 1.33 (s, 3H), 1.18 (s, 6H) ppm. MS: M/e 467 (M+1)⁺.

Compound A29b (the later peak): ¹H NMR (400 MHz, CD₃OD) δ 7.92 (s, 1H), 7.62 (s, 1H), 7.13-7.11 (d, J=8 Hz, 1H), 7.06-7.04 (m, 1H), 5.56 (s, 1H), 5.47 (s, 2H), 4.66-4.59 (m, 4H), 3.94 (s, 1H), 3.43-3.42 (m, 7H), 3.01-2.89 (m, 3H), 1.40-1.39 (d, J=4 Hz, 3H), 1.30-1.29 (d, J=4 Hz, 3H), 1.00-0.99 (d, J=4 Hz, 3H) ppm. MS: M/e 467 (M+1)⁺.

### Compound A46: mixture of 2-(7-((2S,5R)-4-(1-(3,3-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile, and 2-(7-((2S,5R)-4-(1-(2,2-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: mixture of 5-bromo-2-((2-methylallyl)oxy)phenol and 4-bromo-2-((2-methylallyl)oxy)phenol

To a solution of 4-bromobenzene-1,2-diol (10.0 g, 52.9 mmol) and Li₂CO₃ (5.8 g, 79.4 mmol) in DMF (50 mL) was added a solution of 3-bromo-2-methylprop-1-ene (8.6 g, 63.7 mmol) in DMF (50 mL) and the mixture was stirred at 60° C. for 2 days. The mixture was cooled and the resulting suspension was filtered. The filtrate was treated with EA (200 mL) and H₂O (200 mL), then acidized with HCl aq. (1 M) to pH ˜5. Organic layers were separated and the aqueous layer was extracted with EA (50 mL×2). The combined organic layers were washed with brine (100 mL×3), dried over Na₂SO₄ and concentrated to dryness. The resulting residue was purified by flash column chromatography to give the titled compound of positional isomers (4.7 g, 37%) as a mixture. MS: M/e 243, 245 (M+1)⁺.

### Step B: mixture of 7-bromo-2,2-dimethyl-2,3-dihydrobenzo[b][1,4]dioxine and 6-bromo-2,2-dimethyl-2,3-dihydrobenzo[b][1,4]dioxine

The solution of the mixed product of 5-bromo-2-((2-methylallyl)oxy)phenol and 4-bromo-2-((2-methylallyl)oxy)phenol in HCOOH (50 mL) was refluxed for 5 hours. The mixture was concentrated to dryness. The resulting residue was diluted with EA (100 mL), washed with saturated NH₄Cl (50 mL×2), saturated NaHCO₃ aq. (50 mL), brine (50 mL×2), dried and concentrated to dryness. The resulting residue was purified by flash column chromatography to give the titled compound (1.5 g, 33%) as a mixture. MS: M/e 243, 245 (M+1)⁺.

### Step C: mixture of 1-(3,3-dimethyl-2,3-dihydrobenzo[b][1,4]dioxin-6-yl)ethan-1-one and 1-(2,2-dimethyl-2,3-dihydrobenzo[b][1,4]dioxin-6-yl)ethan-1-one

A mixture of 7-bromo-2,2-dimethyl-2,3-dihydrobenzo[b][1,4]dioxine and 6-bromo-2,2-dimethyl-2,3-dihydrobenzo[b][1,4]dioxine (1.4 g, 5.76 mmol), tributyl(1-ethoxyvinyl)stannane (4.1 g, 11.3 mmol) and Pd(PPh₃)₂Cl₂ (200 mg, 0.286 mmol) in toluene (20 mL) was stirred at 100° C. under N₂ for 16 hours. The mixture was diluted with EA (20 mL) and H₂O (20 mL). The suspension was filtered through a celite pad. The aqueous layer was extracted with EA (10 mL×2). The combined organic layers were treated with HCl (5 mL, 4 M in 1,4-dioxane), stirred for 5 minutes. The resulting mixture was washed with brine (20 mL), saturated NaHCO₃ aq. (20 mL×2), brine (20 mL), dried and concentrated to dryness. The resulting oil was purified by flash column chromatography to give the titled compound (630 mg, 53%) as a mixture. MS: M/e 207 (M+1)⁺.

### Step D: mixture of 1-(3,3-dimethyl-2,3-dihydrobenzo[b][1,4]dioxin-6-yl)ethan-1-ol and 1-(2,2-dimethyl-2,3-dihydrobenzo[b][1,4]dioxin-6-yl)ethan-1-ol

To a solution of the mixture of 1-(3,3-dimethyl-2,3-dihydrobenzo[b][1,4]dioxin-6-yl)ethan-1-one and 1-(2,2-dimethyl-2,3-dihydrobenzo[b][1,4]dioxin-6-yl)ethan-1-one (630 mg, 3.06 mmol) in MeOH (10 mL) was added NaBH₄ (232 mg, 6.12 mmol) at room temperature and the mixture was stirred at room temperature for 16 hours. The mixture was treated with saturated NaHCO₃ aq., extracted with EA (10 mL×3). The combined organic layers were washed with brine (10 mL×2), dried over Na₂SO₄ and concentrated to dryness. The resulting residue was purified by flash column chromatography to give the titled compound (620 mg, 97%) as a mixture. MS: M/e 209 (M+1)⁺.

### Step E: mixture of 7-((2S,5R)-4-(1-(3,3-dimethyl-2,3-dihydrobenzo[b][1,4]dioxin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one and 7-((2S,5R)-4-(1-(2,2-dimethyl-2,3-dihydrobenzo[b][1,4]dioxin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

To a solution of the mixture of 1-(3,3-dimethyl-2,3-dihydrobenzo[b][1,4]dioxin-6-yl)ethan-1-ol and 1-(2,2-dimethyl-2,3-dihydrobenzo[b][1,4]dioxin-6-yl)ethan-1-ol (180 mg, 0.86 mmol), Intermediate 10B (130 mg, 0.38 mmol) and DIPEA (350 mg, 2.7 mmol) in MeCN (2 mL) was stirred at 100° C. for 16 hours. The mixture was diluted with EA (20 mL), washed with brine (10 mL×3), dried, concentrated. The resulting residue was purified by Prep-TLC (CH₂Cl₂/MeOH=15/1) to give the titled compound (180 mg, 88%) as a mixture. MS: M/e 536 (M+1)⁺.

### Step F: mixture of 7-((2S,5R)-4-(1-(3,3-dimethyl-2,3-dihydrobenzo[b][1,4]dioxin-6-yl)ethyl)-2,5-dimethylpiperazin-1-yl)-4-methyl-2,4-dihydro-5H-pyrazolo[4,3-b]pyridin-5-one and 7-((2S,5R)-4-(1-(2,2-dimethyl-2,3-dihydrobenzo[b][1,4]dioxin-6-yl)ethyl)-2,5-dimethylpiperazin-1-yl)-4-methyl-2,4-dihydro-5H-pyrazolo[4,3-b]pyridin-5-one

To a stirred mixture of [7-((2S,5R)-4-(1-(3,3-dimethyl-2,3-dihydrobenzo[b][1,4]dioxin-6-yl)ethyl)-2,5-dimethylpiperazin-1-yl)-4-methyl-2,4-dihydro-5H-pyrazolo[4,3-b]pyridin-5-one and 7-((2S,5R)-4-(1-(2,2-dimethyl-2,3-dihydrobenzo[b][1,4]dioxin-6-yl)ethyl)-2,5-dimethylpiperazin-1-yl)-4-methyl-2,4-dihydro-5H-pyrazolo[4,3-b]pyridin-5-one] (160 mg, 0.3 mmol) in MeOH (3 mL) was added HCl (3 mL, 4 M in 1,4-dioxane). The resulting mixture was stirred at room temperature for 16 hours. The reaction mixture was concentrated to dryness to give the titled compound (180 mg, crude) as a mixture. MS: M/e 452 (M+1)⁺.

### Step G: mixture of 2-(7-((2S,5R)-4-(1-(3,3-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile and 2-(7-((2S,5R)-4-(1-(2,2-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of [a mixture of 7-((2S,5R)-4-(1-(3,3-dimethyl-2,3-dihydrobenzo[b][1,4]dioxin-6-yl)ethyl)-2,5-dimethylpiperazin-1-yl)-4-methyl-2,4-dihydro-5H-pyrazolo[4,3-b]pyridin-5-one and 7-((2S,5R)-4-(1-(2,2-dimethyl-2,3-dihydrobenzo[b][1,4]dioxin-6-yl)ethyl)-2,5-dimethylpiperazin-1-yl)-4-methyl-2,4-dihydro-5H-pyrazolo[4,3-b]pyridin-5-one] (170 mg, 0.37 mmol), K₂CO₃ (200 mg, 1.45 mmol) and H₂O (0.5 mL) in MeCN (3 mL) was added 2-iodoacetonitrile (100 mg, 0.60 mmol). The resulting mixture was stirred at room temperature for 16 hours. The mixture was diluted with 5 mL of brine, extracted with EA (5 mL×3). The combined organic layers were washed with brine (5 mL×2), dried over Na₂SO₄, and concentrated to dryness. The resulting residue was purified by Prep-TLC (CH₂Cl₂:MeOH=15:1) to give two fractions, named as Compound A46a (1.5 mg) and Compound A46b (24 mg).

Compound A46a (the earlier peak): ¹H NMR (400 MHz, CD₃OD) δ 7.92 (s, 1H), 7.00-6.86 (m, 2H), 6.82-6.70 (m, 1H), 6.24 (s, 1H), 5.93-5.69 (m, 1H), 5.50 (d, J=18.0 Hz, 1H), 3.95-3.83 (m, 2H), 3.60 (s, 3H), 3.48-3.35 (m, 1H), 3.28-3.03 (m, 2H), 2.95-2.74 (m, 1H), 2.74-2.54 (m, 2H), 2.39-2.25 (m, 1H), 1.34-1.30 (m, 9H), 1.21 (d, J=6.0 Hz, 3H), 0.96 (d, J=6.0 Hz, 3H) ppm. MS: M/e 491 (M+1)⁺.

Compound A46b (the later peak): ¹H NMR (400 MHz, CD₃OD) δ 7.92 (d, J=1.2 Hz, 1H), 6.96-6.66 (m, 3H), 5.55 (d, J=1.6 Hz, 1H), 5.47 (d, J=2.0 Hz, 2H), 4.87-4.18 (m, 2H), 3.88 (d, J=6.0 Hz, 2H), 3.63-3.52 (m, 1H), 3.46-3.37 (m, 4H), 3.03-2.90 (m, 1H), 2.87-2.23 (m, 2H), 1.42-1.30 (m, 9H), 1.29-1.18 (m, 3H), 1.15-0.93 (m, 3H) ppm. MS: M/e 491 (M+1)⁺.

Another batch 150 mg Compound A46 as a mixture of 2-(7-((2S,5R)-4-(1-(3,3-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile and 2-(7-((2S,5R)-4-(1-(2,2-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile was prepared according to the procedures as above described for Compound A46 that could be recognized by one skilled in the art.

Compound A46 (150 mg) as a mixture was further separated by twice chiral prep-HPLC conditions into 4 isomers respectively, Compound A46c (9 mg), Compound A46d (9 mg), Compound A46e (14 mg) and Compound A46f (10 mg). The chiral separation conditions are shown below.

### The First Prep-HPLC Condition

| Column | CHIRALPAK IH | |
|---|---|---|
| Column Size | 5 cm × 25 cm, 5 um | |
| Mobile Phase A | MtBE(20 mmol/L NH3 MeOH) | |
| Mobile Phase B | EtOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

### The Second Prep-HPLC Condition

| Column | CHIRAL Cellulose-SB | |
|---|---|---|
| Column Size | 5 cm × 25 cm, 5 um | |
| Mobile Phase A | Hex(20 mmol/L NH3 MeOH) | |
| Mobile Phase B | EtOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A46c (the first peak): ¹HNMR (400 MHz, CD₃OD) δ 7.91 (s, 1H), 6.88 (d, J=1.6 Hz, 1H), 6.87-6.80 (m, 1H), 6.74 (d, J=8.0 Hz, 1H), 5.55 (s, 1H), 5.47 (s, 2H), 4.30 (s, 1H), 3.89 (s, 2H), 3.60-3.49 (m, 1H), 3.46-3.37 (m, 4H), 3.37-3.31 (m, 1H), 3.04-2.91 (m, 2H), 2.86-2.74 (m, 1H), 1.38 (d, J=6.4 Hz, 3H), 1.32 (s, 6H), 1.29-1.26 (m, 3H), 0.98 (d, J=6.4 Hz, 3H) ppm. MS: M/e 491 (M+1)⁺.

Compound A46d (the second peak): ¹HNMR (400 MHz, CD₃OD) δ 7.92 (s, 1H), 6.88-6.76 (m, 3H), 5.55 (s, 1H), 5.47 (s, 2H), 4.57 (s, 1H), 4.27 (s, 1H), 3.88 (s, 2H), 3.57-3.46 (m, 1H), 3.45-3.36 (m, 4H), 3.02-2.89 (m, 2H), 2.88-2.74 (m, 1H), 1.38 (d, J=6.8 Hz, 3H), 1.33 (s, 6H), 1.27 (d, J=6.8 Hz, 3H), 0.97 (d, J=6.4 Hz, 3H) ppm. MS: M/e 491 (M+1)⁺.

Compound A46e (the third peak): ¹HNMR (400 MHz, CD₃OD) δ 7.93 (s, 1H), 7.00-6.79 (m, 2H), 6.79-6.65 (m, 1H), 5.57 (s, 1H), 5.48 (s, 2H), 4.71-4.42 (m, 2H), 3.89 (s, 2H), 3.70-3.52 (m, 2H), 3.43 (s, 3H), 3.04-2.52 (m, 2H), 2.47-2.22 (m, 1H), 1.33-1.28 (m, 9H), 1.22 (d, J=6.0 Hz, 3H), 1.19-1.06 (m, 3H) ppm. MS: M/e 491 (M+1)⁺.

Compound A46f (the fourth peak): ¹HNMR (400 MHz, CD₃OD) δ 7.92 (s, 1H), 6.88-6.72 (m, 3H), 5.56 (s, 1H), 5.47 (s, 2H), 4.65-4.85 (m, 1H), 4.57 (s, 1H), 3.87 (s, 2H), 3.69-3.51 (m, 2H), 3.49-3.34 (m, 4H), 2.71 (dd, J=12.0, 3.6 Hz, 1H), 2.27 (d, J=12.8 Hz, 1H), 1.32-1.28 (m, 9H), 1.21 (d, J=6.4 Hz, 3H), 1.11 (d, J=6.4 Hz, 3H) ppm. MS: M/e 491 (M+1)⁺.

The Compound A46d and Compound A46f can also be synthesized through another method as following: Mixture of Compound A46d and Compound A46f: 2-(7-((2S,5R)-4-(1-(3,3-dimethyl-2,3-dihydrobenzo[b] [1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile.

### Step A: 1-(4-bromo-2-((4-methoxybenzyl)oxy)phenyl)ethan-1-one

To a mixture of 1-(4-bromo-2-hydroxyphenyl)ethan-1-one (10.0 g, 46.7 mmol), K₂CO₃ (12.9 g, 93.6 mmol) in DMF (80 mL) was added 1-(chloromethyl)-4-methoxybenzene (7.3 g, 46.7 mmol) at room temperature and the mixture was stirred for 16 hrs. The mixture was added H₂O (200 mL) and extracted with EtOAc (100 mL×3). The combined extracts were washed with brine (100 mL×3), dried over Na₂SO₄, filtered and concentrated to dryness. The resulting residue was purified by column chromatography to give the titled compound (13.8 g, 88%). ¹H NMR (400 MHz, DMSO-d₆) δ 7.58-7.48 (m, 2H), 7.44 (d, J=8.4 Hz, 2H), 7.23 (dd, J=8.4, 1.6 Hz, 1H), 6.97 (d, J=8.8 Hz, 2H), 5.19 (s, 2H), 3.77 (s, 3H), 2.45 (s, 3H) ppm.

### Step B: 4-bromo-2-((4-methoxybenzyl)oxy)phenyl acetate

To a stirred solution of 1-(4-bromo-2-((4-methoxybenzyl)oxy)phenyl)ethan-1-one (12.8 g, 38.3 mmol) in CH₂Cl₂ (150 mL) was added 3-chlorobenzoperoxoic acid (22.0 g, 95.6 mmol) in portions at room temperature and the resulting mixture was stirred at room temperature for 64 hrs. The suspension was filtered and the filtrate was washed with NaHCO₃ (50 mL×3), brine (50 mL×2), dried over Na₂SO₄, filtered and concentrated to dryness to give the titled compound (13.5 g, crude).

### Step C: 4-bromo-2-((4-methoxybenzyl)oxy)phenol

To a solution of 4-bromo-2-((4-methoxybenzyl)oxy)phenyl acetate (13.0 g, 37 mmol) in THF (100 mL) was added an aqueous solution of NaOH (4M, 50 mL) at room temperature and stirred for 2 hrs. The mixture was acidified with HCl (2 M) to pH ˜3. The mixture was extracted with EtOAc (100 mL×2). The combined extracts were washed with brine (100 mL×2), dried over Na₂SO₄, filtered and concentrated. The resulting residue was purified by flash column chromatography to give the titled compound (6.8 g, 60% for 2 steps).

¹H NMR (400 MHz, DMSO-d₆) δ 9.29 (s, 1H), 7.39 (d, J=8.4 Hz, 2H), 7.13 (d, J=2.0 Hz, 1H), 7.00-6.85 (m, 3H), 6.74 (d, J=8.4 Hz, 1H), 5.02 (s, 2H), 3.76 (s, 3H) ppm.

### Step D: 4-bromo-2-((4-methoxybenzyl)oxy)-1-((2-methylallyl)oxy)benzene

To a mixture of 4-bromo-2-((4-methoxybenzyl)oxy)phenol (6.8 g, 22.1 mmol), K₂CO₃ (9.1 g, 66.3 mmol) in DMF (50 mL) was added 3-bromo-2-methylprop-1-ene (8.6 g, 63.7 mmol) at room temperature and the mixture was stirred for 5 hrs. The mixture was diluted with EtOAc (100 mL), washed with brine (50 mL×3), dried over Na₂SO₄, filtered and concentrated to dryness. The resulting residue was purified by flash column chromatography to give the titled compound (6.71 g, 84%). ¹H NMR (400 MHz, DMSO-d₆) δ 7.37 (d, J=8.4 Hz, 2H), 7.22 (d, J=2.4 Hz, 1H), 7.04 (dd, J=8.4, 2.0 Hz, 1H), 6.99-6.89 (m, 3H), 5.04 (s, 2H), 5.02 (s, 1H), 4.92 (s, 1H), 4.46 (s, 2H), 3.76 (s, 3H), 1.74 (s, 3H) ppm.

### Step E: 5-bromo-2-((2-methylallyl)oxy)phenol

A solution of 4-bromo-2-((4-methoxybenzyl)oxy)-1-((2-methylallyl)oxy)benzene (5.7 g, 15.7 mmol) in acetic acid (50 mL) was stirred at 100° C. for 6 hrs. The mixture was concentrated to dryness and the residue was diluted with EtOAc (100 mL), washed with NaHCO₃ (aq. 20 mL×2), brine (20 mL), dried over Na₂SO₄, filtered and concentrated to dryness. The resulting residue was purified by flash column chromatography (120 g of silica-gel column) to give the titled compound (3.4 g, 89%). ¹H NMR (400 MHz, DMSO-d₆) δ 9.47 (s, 1H), 6.96-6.92 (m, 1H), 6.89-6.80 (m, 2H), 5.05 (s, 1H), 4.93 (s, 1H), 4.44 (s, 2H), 1.76 (s, 3H) ppm. MS: M/e 243 (M+1)⁺

### Step F: 7-bromo-2,2-dimethyl-2,3-dihydrobenzo[b][1,4]dioxine

The solution of 5-bromo-2-((2-methylallyl)oxy)phenol (3.36 g, 13.9 mmol) in HCOOH (60 mL) was stirred at 100° C. for 2 hrs. The mixture was concentrated to dryness and the resulting residue was diluted with EA (50 mL), washed with H₂O (20 mL×3), NaHCO₃ (20 mL×2), brine (20 mL×2), dried, concentrated and purified by flash column chromatography to give the titled compound (980 mg, 29%). ¹H NMR (400 MHz, CDCl₃) δ 6.97 (d, J=2.0 Hz, 1H), 6.95-6.89 (m, 1H), 6.75 (d, J=8.4 Hz, 1H), 3.86 (s, 2H), 1.33 (s, 6H) ppm.

### Step G: 1-(3,3-dimethyl-2,3-dihydrobenzo[b][1,4]dioxin-6-yl)ethan-1-one

A mixture of 7-bromo-2,2-dimethyl-2,3-dihydrobenzo[b][1,4]dioxine (980 mg, 4.05 mmol), tributyl(1-ethoxyvinyl)stannane (2.17 g, 6.01 mmol) and Pd(PPh₃)₂Cl₂ (140 mg, 0.20 mmol) in toluene (20 mL) was stirred at 100° C. under N₂ for 16 hrs. The mixture was cooled and diluted with EA (40 mL) and 20 mL of H₂O. The suspension was filtered through a celite pad. The organic lay was treated with HCl/Dioxane (4M, 3 mL), stirred for 2 min. The resulting mixture was washed with brine (20 mL×2), NaHCO₃ (20 mL×2), brine (20 mL), dried over Na₂SO₄, filtered and concentrated to dryness. The resulting residue was purified by flash column chromatography to give the titled compound (530 mag 64%). ¹H NMR (400 MHz, CDCl₃) δ 7.54-7.44 (m, 2H), 6.92 (d, J=8.0 Hz, 1H), 3.94 (s, 2H), 2.53 (s, 3H), 1.36 (s, 6H) ppm.

### Step H: 1-(3,3-dimethyl-2,3-dihydrobenzo[b][1,4]dioxin-6-yl)ethan-1-ol

To a solution of 1-(3,3-dimethyl-2,3-dihydrobenzo[b][1,4]dioxin-6-yl)ethan-1-one (530 mg, 2.57 mmol) in MeOH (5 mL) was added NaBH₄ (98 mg, 2.57 mmol) at 0° C. and the mixture was stirred at RT for 30 min. The mixture was treated with NaHCO₃ (20 mL), extracted with EA (10 mL×3). The combined extracts was washed with brine (10 mL×2), dried over Na₂SO₄ and concentrated to dryness to give the titled compound (515 mg, 96%). ¹H NMR (400 MHz, DMSO-d₆) δ 6.83-6.71 (m, 3H), 4.99 (d, J=4.4 Hz, 1H), 4.66-4.49 (m, 1H), 3.88 (s, 2H), 1.28-1.24 (m, 9H).

### Step I: 2-(7-((2S,5R)-4-(1-(3,3-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 1-(3,3-dimethyl-2,3-dihydrobenzo[b][1,4]dioxin-6-yl)ethan-1-ol (208 mg, 1.0 mmol), Intermediate 10 (210 mg, 0.7 mmol) and (cyanomethyl)trimethylphosphonium iodide (510 mg, 2.1 mmol) in CH₃CN (2 mL) was added DIPEA (540 mg, 4.2 mmol). The mixture solution was diluted with EA (20 mL), washed with brine (10 mL×3), dried over Na₂SO₄, filtered and concentrated under reduced pressure. The resulting residue was purified by flash column chromatography and further purified by Prep-HPLC to give the titled compound (60 mg, 17%, as a mixture), which was further separated into Compound A46d (14 mg) and Compound A46f (16 mg) by chiral Prep-HPLC. The chiral separation conditions are shown below.

| Column | CHIRALPAK IH | |
|---|---|---|
| Column Size | 2 cm × 25 cm, 5 um | |
| Mobile Phase A | MtBE | |
| Mobile Phase B | EtOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A46d (the earlier peak): ¹HNMR (400 MHz, CD₃OD) δ 7.92 (s, 1H), 6.88-6.76 (m, 3H), 5.55 (s, 1H), 5.47 (s, 2H), 4.57 (s, 1H), 4.27 (s, 1H), 3.88 (s, 2H), 3.57-3.46 (m, 1H), 3.45-3.36 (m, 4H), 3.02-2.89 (m, 2H), 2.88-2.74 (m, 1H), 1.38 (d, J=6.8 Hz, 3H), 1.33 (s, 6H), 1.27 (d, J=6.8 Hz, 3H), 0.97 (d, J=6.4 Hz, 3H) ppm. MS: M/e 491 (M+1)⁺.

Compound A46f: (the later peak): ¹HNMR (400 MHz, CD₃OD) δ 7.92 (s, 1H), 6.88-6.72 (m, 3H), 5.56 (s, 1H), 5.47 (s, 2H), 4.65-4.85 (m, 1H), 4.57 (s, 1H), 3.87 (s, 2H), 3.69-3.51 (m, 2H), 3.49-3.34 (m, 4H), 2.71 (dd, J=12.0, 3.6 Hz, 1H), 2.27 (d, J=12.8 Hz, 1H), 1.32-1.28 (m, 9H), 1.21 (d, J=6.4 Hz, 3H), 1.11 (d, J=6.4 Hz, 3H) ppm. MS: M/e 491 (M+1)⁺.

Compound A47: 2-(7-((2S,5R)-2,5-diethyl-4-(1-(quinoxalin-6-yl)ethyl)piperazin-1-yl)-4-ethyl-5-oxo-4,5-dihydro-2H-pyrazolo[4,3-b]pyridin-2-yl)acetonitrile

### Step A: ethyl 4-acetamido-1-(tetrahydro-2H-pyran-2-yl)-1H-pyrazole-3-carboxylate

A mixture of ethyl 4-((cyclopropylmethyl)amino)-1-(tetrahydro-2H-pyran-2-yl)-1H-pyrazole-3-carboxylate (200 mg, 0.8368 mmol), acetic anhydride (2 mL) in pyridine (5 mL) was stirred at RT overnight. The mixture was concentrated, extracted with DCM (100 mL) and washed with water. The organic layer was dried and concentrated to dryness. The resulting residue was purified by flash column chromatography (PE:EA=1:1) to give the titled compound (200 mg, 85.05%). MS: M/e 282 (M+1)⁺

### Step B: ethyl 4-(N-ethylacetamido)-1-(tetrahydro-2H-pyran-2-yl)-1H-pyrazole-3-carboxylate

A mixture of ethyl 4-acetamido-1-(tetrahydro-2H-pyran-2-yl)-1H-pyrazole-3-carboxylate (200 mg, 0.7117 mmol), iodoethane (167 mg, 1.068 mmol) and Cs₂CO₃ (464 mg, 1.423 mmol) in DMF (5 mL) was stirred at 60° C. overnight. The mixture was concentrated, extracted with DCM (100 mL) and washed with water. The organic layer was dried and concentrated to dryness. The resulting residue was purified by flash column chromatography (PE:EA=1:1) to give the titled compound (200 mg, 90.94%). MS: M/e 310 (M+1)⁺

### Step C: 4-ethyl-7-hydroxy-2-(tetrahydro-2H-pyran-2-yl)-2,4-dihydro-5H-pyrazolo[4,3-b]pyridin-5-one

To a solution of ethyl 4-(N-ethylacetamido)-1-(tetrahydro-2H-pyran-2-yl)-1H-pyrazole-3-carboxylate (200 mg, 0.6473 mmol) in THF under N₂ was added L1HMDS (1 mL, 1 mmol) dropwise at −78° C. and stirred for 2 hours. The mixture was quenched with AcOH, extracted with DCM (80 mL) and washed with water. The organic layer was dried and concentrated to dryness. The resulting residue was purified by flash column chromatography (DCM:MeOH=10:1) to give the titled compound (130 mg, 49.43%). MS: M/e 264 (M+1)⁺

### Step D: 4-ethyl-5-oxo-2-(tetrahydro-2H-pyran-2-yl)-4,5-dihydro-2H-pyrazolo[4,3-b]pyridin-7-yl trifluoromethanesulfonate

A mixture of 4-ethyl-7-hydroxy-2-(tetrahydro-2H-pyran-2-yl)-2,4-dihydro-5H-pyrazolo[4,3-b]pyridin-5-one (130 mg, 0.4943 mmol), 1,1,1-trifluoro-N-phenyl-N-((trifluoromethyl)sulfonyl)methanesulfonamide (265 mg, 0.7414 mmol), K₂CO₃ (134 mg, 0.9886 mmol) in DMF (5 mL) was stirred under N₂ at RT overnight. The mixture was extracted with DCM (50 mL) and washed with water. The organic layer was dried and concentrated to dryness. The resulting residue was purified by flash column chromatography (DCM:MeOH=20:1) to give the titled compound (150 mg, 76.83%). MS: M/e 396 (M+1)+

### Step E: 7-((2S,5R)-2,5-diethyl-4-(1-(quinoxalin-6-yl)ethyl)piperazin-1-yl)-4-ethyl-2-(tetrahydro-2H-pyran-2-yl)-2,4-dihydro-5H-pyrazolo[4,3-b]pyridin-5-one

A mixture of 4-ethyl-5-oxo-2-(tetrahydro-2H-pyran-2-yl)-4,5-dihydro-2H-pyrazolo[4,3-b]pyridin-7-yl trifluoromethanesulfonate (150 mg, 0.3796 mmol), 6-(1-((2R,5S)-2,5-diethylpiperazin-1-yl)ethyl)quinoxaline (170 mg, 0.5696 mmol), DIPEA (245 mg, 1.899 mmol) in CH₃CN (5 mL) was stirred under N₂ at 100° C. overnight. The mixture was extracted with DCM (50 mL) and washed with water. The organic layer was dried and concentrated to dryness. The resulting residue was purified by Prep-TLC (DCM:MeOH=20:1) to give the titled compound (60 mg, 29.10%). MS: M/e 544 (M+1)⁺

### Step F: 7-((2S,5R)-2,5-diethyl-4-(1-(quinoxalin-6-yl)ethyl)piperazin-1-yl)-4-ethyl-2,4-dihydro-5H-pyrazolo[4,3-b]pyridin-5-one

A mixture of 7-((2S,5R)-2,5-diethyl-4-(1-(quinoxalin-6-yl)ethyl)piperazin-1-yl)-4-ethyl-2-(tetrahydro-2H-pyran-2-yl)-2,4-dihydro-5H-pyrazolo[4,3-b]pyridin-5-one (60 mg, 0.1105 mmol) in TFA (2 mL) and DCM (2 mL) was stirred at RT overnight. The solution was concentrated and purified by Prep-TLC (DCM:MeOH=20:1) to give the titled compound (50 mg, 98.58%). MS: M/e 460 (M+1)⁺

### Step G: 2-(7-((2S,5R)-2,5-diethyl-4-(1-(quinoxalin-6-yl)ethyl)piperazin-1-yl)-4-ethyl-5-oxo-4,5-dihydro-2H-pyrazolo[4,3-b]pyridin-2-yl)acetonitrile

A mixture of 7-((2S,5R)-2,5-diethyl-4-(1-(quinoxalin-6-yl)ethyl)piperazin-1-yl)-4-ethyl-2,4-dihydro-5H-pyrazolo[4,3-b]pyridin-5-one (50 mg, 0.1089 mmol), 2-iodoacetonitrile (36 mg, 0.2179 mmol), K₂CO₃ (45 mg, 0.3268 mmol) in DMF (3 mL) was stirred at RT for 5 hours. The mixture was extracted with DCM (50 mL) and washed with water. The organic layer was dried and concentrated to dryness. The resulting residue was purified by Prep-TLC (DCM:MeOH=20:1) to give the titled compound (18 mg, 33.18%). ¹H NMR (400 MHz, DMSO-d6) δ 8.94 (s, 2H), 8.14-8.01 (m, 3H), 7.96 (s, 1H), 5.60 (s, 2H), 5.38 (s, 1H), 4.05 (s, 1H), 3.94-3.77 (m, 3H), 3.21-2.93 (m, 3H), 2.88-2.61 (m, 2H), 1.66-1.48 (m, 3H), 1.41-1.33 (m, 3H), 1.12 (s, 3H), 1.06-0.78 (m, 4H), 0.68-0.40 (m, 3H) ppm. MS: M/e 499 (M+1)⁺

### Compound A52: 2-(7-((2S,5R)-4-(1-(benzo[d]thiazol-2-yl)ethyl)-2,5-diethylpiperazin-1-yl)-4-methyl-5-oxo-4,5-dihydro-2H-pyrazolo[4,3-b]pyridin-2-yl)acetonitrile

### Step A: 1-(benzo[d]thiazol-2-yl)ethan-1-ol

A mixture of 2-aminobenzenethiol (1.25 g, 10 mmol) and 2-hydroxypropanoic acid (1.08 g, 12 mmol) in HCL (30 mL, 4M) was heated to reflux for overnight. After cooling to room temperature, the reaction mixture was poured into water. Next, the pH was adjusted to 10-11 with ammonia. The mixture was filtered, and The resulting residue was recrystallized from water to give the titled compound (450 mg, 25%). MS: M/e 180 (M+1)⁺.

### Step B: tert-butyl (2S,5R)-4-(1-(benzo[d]thiazol-2-yl)ethyl)-2,5-diethylpiperazine-1-carboxylate

A mixture of tert-butyl (2S,5R)-2,5-diethylpiperazine-1-carboxylate (170 mg, 0.7 mmol), 1-(benzo[d]thiazol-2-yl)ethan-1-ol (200 mg, 1.1 mmol), (cyanomethyl)trimethylphosphonium iodide (97 mg, 1.4 mmol) and DIPEA (450 mg, 3.5 mmol) in CH₃CN (2 mL) was heated to 105° C. for overnight under N₂ atmosphere. The solvent was removed under vacuum. The crude product was purified by silica flash column chromatography (DCM:MeOH=100:1) to give the titled compound (280 mg, 79%, 80% purity). MS: M/e 403 (M+1)⁺.

### Step C: 2-(1-((2R,5S)-2,5-diethylpiperazin-1-yl)ethyl)benzo[d]thiazole

To a solution of tert-butyl (2S,5R)-4-(1-(benzo[d]thiazol-2-yl)ethyl)-2,5-diethylpiperazine-1-carboxylate (280 mg, 80% purity) in DCM (5 mL) were added TFA (2 mL) at room temperature. The resulting mixture was stirred at room temperature for another 2 hours. The reaction solvent was removed under vacuum. The crude product was dissolved with DCM (mL) and neutralized with aq. NaOH to pH=8. The organic layer was concentrated to give the titled compound (160 mg, crude) which was used in the next step directly without further purification. MS: M/e 295 (M+1)⁺.

### Step D: 7-((2S,5R)-4-(1-(benzo[d]thiazol-2-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one

A mixture of Intermediate 2 (77 mg, 0.2 mmol), 2-(1-((2R,5S)-2,5-diethylpiperazin-1-yl)ethyl)benzo[d]thiazole (60 mg, 0.2 mmol) and DIPEA (128 mg, 1 mmol) in CH₃CN was heated to 105° C. for overnight under N₂ atmosphere. The solvent was removed under vacuum. The crude product was purified by Prep-TLC (DCM:MeOH=15:1) to give the titled compound (50 mg, 47%). MS: M/e 535 (M+1)⁺.

### Step E: 7-((2S,5R)-4-(1-(benzo[d]thiazol-2-yl)ethyl)-2,5-diethylpiperazin-1-yl)-4-methyl-2,4-dihydro-5H-pyrazolo[4,3-b]pyridin-5-one

To a solution of 7-((2S,5R)-4-(1-(benzo[d]thiazol-2-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one (50 mg, 0.09 mmol) in MeOH (2 mL) was added HCl dioxane solution (0.5 mL, 2 mmol, 4M) at room temperature. The resulting mixture was stirred at room temperature for another 2 hours. The reaction solvent was removed under vacuum. The crude product was dissolved with DCM (10 mL) and neutralized with aq·NaOH to pH=8. The organic layer was concentrated to give crude product which further purified by Prep-TLC (DCM:MeOH=10:1) to give the titled compound (15 mg, 37%). MS: M/e 451 (M+1)⁺.

### Step F: 7-2-(7-((2S,5R)-4-(1-(benzo[d]thiazol-2-yl)ethyl)-2,5-diethylpiperazin-1-yl)-4-methyl-5-oxo-4,5-dihydro-2H-pyrazolo[4,3-b]pyridin-2-yl)acetonitrile

To a solution of the compound of Step E (15 mg, 0.03 mmol) and K₂CO₃ (4.6 mg, 0.06 mmol) in DMF (2 mL) were added 2-iodoacetonitrile (8.5 mg, 0.05 mmol). The resulting mixture was stirred at room temperature overnight. The reaction mixture was diluted with EA (10 mL) and washed with brine (10 mL×3). The organic layers were concentrated under reduced pressure. The resulting residue was purified by Prep-TLC (DCM:MeOH=15:1) to give the titled compound (8 mg, 48%). ¹H NMR (400 MHz, CD₃OD) δ 7.95-7.90 (m, 3H), 7.49-7.41 (m, 2H), 5.61-5.58 (m, 1H), 5.48 (s, 2H), 4.37 (br s, 1H), 3.48-3.46 (m, 1H), 3.45 (s, 3H), 3.15-3.10 (m, 2H), 2.94-2.68 (m, 3H), 2.20-2.16 (m, 1H), 1.98-1.90 (m, 3H), 1.65-1.54 (m, 3H), 1.05-0.78 (m, 6H) ppm. MS: M/e 490 (M+1)⁺.

### Compound A62: 2-(7-((2S,5R)-2,5-diethyl-4-(1-(isoquinolin-7-yl)ethyl)piperazin-1-yl)-4-methyl-5-oxo-4,5-dihydro-2H-pyrazolo[4,3-b]pyridin-2-yl)acetonitrile

### Step A: 1-(isoquinolin-7-yl)ethan-1-one

To a solution of 7-bromoisoquinoline (4.9 g, 23.55 mmol) and tributyl(1-ethoxyvinyl)stannane (11 g, 30.6 mmol) in toluene (60 mL) was added Pd(PPH₃)₂Cl₂ (1.61 g, 2.35 mmol). The mixture was heated at 100° C. for 16 hours under N₂. The reaction mixture was cooled to room temperature, diluted with H₂O/EA, filtered through a pad of Celite, washed with EA. The filtrate was collected, washed with brine, dried over Na₂SO₄, filtered, and concentrated to dryness. The crude was dissolved in THF (80 mL), 3N HCl (20 mL) was added. Then the mixture was stirred at room temperature for 5 h. The reaction mixture was diluted with water, extracted with EA (60 mL×3), washed with brine, dried over Na₂SO₄, filtered, and concentrated to dryness. The resulting residue was purified by flash column chromatography (DCM:MeOH=20:1) to give titled compound (2.3 g, 57%). MS: M/e 172 (M+1)⁺.

### Step B: 1-(isoquinolin-7-yl)ethan-1-ol

To a solution of 1-(isoquinolin-7-yl)ethan-1-one (2.33 g, 13.6 mmol) in MeOH (60 mL) at ice-cooled bath was added NaBH₄ (516 mg, 13.6 mmol) in some portions. The mixture was stirred at room temperature for 2 h. The reaction was quenched with cold water, extracted with DCM (80 mL×2), washed with brine, dried over Na₂SO₄, filtered, and concentrated to dryness. The resulting residue was purified by flash column chromatography (DCM:MeOH=10:1) to give titled compound (1.2 g, 51%). ¹HNMR (400 MHz, DMSO-d6) δ 9.25 (s, 1H), 8.42 (d, J=5.6 Hz, 1H), 8.00 (s, 1H), 7.91-7.85 (m, 1H), 7.80-7.70 (m, 2H), 5.39 (d, J=4.0 Hz, 1H), 4.94-4.81 (m, 1H), 1.37 (d, J=6.4 Hz, 3H) ppm. MS: M/e 174 (M+1)⁺.

### Step C: 2-(7-((2S,5R)-2,5-diethyl-4-(1-(isoquinolin-7-yl)ethyl)piperazin-1-yl)-4-methyl-5-oxo-4,5-dihydro-2H-pyrazolo[4,3-b]pyridin-2-yl)acetonitrile

To a solution of 1-(isoquinolin-7-yl)ethan-1-ol (70 mg, 0.4 mmol), Intermediate 5 (65 mg, 0.2 mmol) and (cyanomethyl)trimethylphosphonium iodide (97 mg, 0.4 mmol) in CH₃CN (2 mL) was added DIPEA (103 mg, 0.8 mmol). The mixture was sealed in a bottle and heated at 100° C. for 16 hours. Another portion of (cyanomethyl)trimethylphosphonium iodide (97 mg, 0.4 mmol) was added and heated at 100° C. for 26 hours. Then the mixture was cooled to room temperature, diluted with water, extracted with EA (50 mL×2), washed with brine, dried over Na₂SO₄, filtered, and concentrated to dryness. The resulting residue was purified by flash column chromatography (DCM:MeOH=15:1) and Prep-HPLC to give titled compound (8 mg, 8%). ¹HNMR (400 MHz, CD₃OD) δ 9.21 (d, J=8.8 Hz, 1H), 8.41 (t, J=5.2 Hz, 1H), 8.11-8.02 (m, 1H), 8.00-7.88 (m, 3H), 7.86-7.78 (m, 1H), 5.59-5.51 (m, 1H), 5.49-5.43 (m, 2H), 4.04-3.96 (m, 0.5H), 3.91-3.81 (m, 0.5H), 3.59-3.50 (m, 0.5H), 3.43 (s, 3H), 3.32-3.28 (m, 2.5H), 3.26-3.17 (m, 0.5H), 3.11-3.03 (m, 0.5H), 2.97-2.86 (m, 0.5H), 2.75-2.64 (m, 0.5H), 2.55-2.30 (m, 1H), 2.21-2.09 (m, 0.5H), 2.06-1.92 (m, 0.5H), 1.91-1.78 (m, 0.5H), 1.76-1.51 (m, 2.5H), 1.49-1.36 (m, 3H), 1.05 (t, J=7.6 Hz, 1.5H), 0.97 (t, J=7.2 Hz, 1.5H), 0.65 (t, J=7.6 Hz, 1.5H), 0.54 (t, J=7.6 Hz, 1.5H) ppm. MS: M/e 484 (M+1)⁺.

### Compound A63: 2-(7-((2S,5R)-4-(1-(1,8-naphthyridin-2-yl)ethyl)-2,5-diethylpiperazin-1-yl)-4-methyl-5-oxo-4,5-dihydro-2H-pyrazolo[4,3-b]pyridin-2-yl)acetonitrile

### Step A: 1-(1,8-naphthyridin-2-yl)ethan-1-ol

To a solution of 1,8-naphthyridine-2-carbaldehyde (600 mg, 3.794 mmol) in THF (30 mL) was degassed 3 times under N₂ atmosphere. Then added CH₃MgBr (1.6 mL, 3M in Et₂O) dropwise at 0° C. The mixture was stirred at 0° C. for 4 hours. The reaction mixture was quenched with saturated NH₄Cl (20 mL) at room temperature and extracted with EA (45 mL×2). The combined organic layers were dried over Na₂SO₄ and concentrated under reduced pressure. The resulting residue was purified by flash column chromatography to give the titled compound (400 mg, 61%). MS: M/e 175 (M+1)⁺.

### Step B: tert-butyl (2S,5R)-4-(1-(1,8-naphthyridin-2-yl)ethyl)-2,5-diethylpiperazine-1-carboxylate

To a solution of 1-(1,8-naphthyridin-2-yl)ethan-1-ol (200 mg, 1.156 mmol), tert-butyl (2S,5R)-2,5-diethylpiperazine-1-carboxylate (281 mg, 1.156 mmol) and (cyanomethyl)trimethylphosphonium iodide (562 mg, 2.312 mmol) in CH₃CN (2 mL) was added DIPEA (747 mg, 5.780 mmol). The mixture solution was degassed 3 times under N₂ atmosphere. Then the mixture solution was stirred at 105° C. for 24 hours. The reaction mixture was concentrated under reduced pressure. The resulting residue was purified by flash column chromatography to give the titled compound (120 mg, 26%). MS: M/e 399 (M+1)⁺.

### Step C: 2-(1-((2R,5S)-2,5-diethylpiperazin-1-yl)ethyl)-1,8-naphthyridine

To a solution of tert-butyl (2S,5R)-4-(1-(1,8-naphthyridin-2-yl)ethyl)-2,5-diethylpiperazine-1-carboxylate (120 mg, 0.300 mmol) in DCM (6 mL) at room temperature was added HCl (2 mL, 4 M in 1,4-dioxane). The mixture was stirred at room temperature for 2 hours. Then the mixture was concentrated under reduced pressure to give the crude product (HCl salt). The crude product was basified by Na₂CO₃ (4M) to pH ˜10 and extracted with EA (30 mL×3). The combined organic layers were washed with brine (20 mL×3), dried over Na₂SO₄ and concentrated under reduced pressure to give the titled compound (60 mg, 66%). MS: M/e 299 (M+1)⁺.

### Step D: 7-((2S,5R)-4-(1-(1,8-naphthyridin-2-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one

To a solution of 2-(1-((2R,5S)-2,5-diethylpiperazin-1-yl)ethyl)-1,8-naphthyridine (60 mg, 0.201 mmol) and Intermediate 2 (92 mg, 0.242 mmol) in CH₃CN (2 mL) at room temperature was added DIPEA (130 mg, 1.005 mmol). The mixture was stirred at 105° C. for 24 hours. Then the mixture was concentrated under reduced pressure to give the crude product. The crude product was purified by flash column chromatography to give the titled compound (80 mg, 75%). MS: M/e 530 (M+1)⁺.

### Step E: 7-((2S,5R)-4-(1-(1,8-naphthyridin-2-yl)ethyl)-2,5-diethylpiperazin-1-yl)-4-methyl-2,4-dihydro-5H-pyrazolo[4,3-b]pyridin-5-one

To a solution of 7-((2S,5R)-4-(1-(1,8-naphthyridin-2-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one (80 mg, 0.151 mmol) in DCM (6 mL) at room temperature was added HCl (3 mL, 4 M in 1,4-dioxane). The mixture was stirred at room temperature for 3 hours. Then the mixture was concentrated under reduced pressure to give the crude product (HCl salt). The crude product was basified by Na₂CO₃ (4M) to pH ˜10 and extracted with EA (35 mL×2). The combined organic layers were washed with brine (20 mL×3), dried over Na₂SO₄ and concentrated under reduced pressure to give the titled compound (50 mg, 75%). MS: M/e 446 (M+1)⁺.

### Step F: 2-(7-((2S,5R)-4-(1-(1,8-naphthyridin-2-yl)ethyl)-2,5-diethylpiperazin-1-yl)-4-methyl-5-oxo-4,5-dihydro-2H-pyrazolo[4,3-b]pyridin-2-yl)acetonitrile

To a solution of 7-((2S,5R)-4-(1-(1,8-naphthyridin-2-yl)ethyl)-2,5-diethylpiperazin-1-yl)-4-methyl-2,4-dihydro-5H-pyrazolo[4,3-b]pyridin-5-one (50 mg, 0.112 mmol) and K₂CO₃ (46 mg, 0.336 mmol) in DMF (5 mL) at room temperature was added 2-iodoacetonitrile (28 mg, 0.169 mmol). The mixture solution was stirred at room temperature for 12 hours. Then the reaction mixture was quenched with saturated NaCl (10 mL) at room temperature and extracted with EA (30 mL×2). The combined organic layers were dried over Na₂SO₄ and concentrated under reduced pressure. The resulting residue was purified by Prep-HPLC to give the titled compound (6 mg, 11%). ¹H NMR (400 MHz, CD₃OD) δ 9.21-9.19 (t, J=4.7 Hz, 1H), 8.69-8.59 (m, 2H), 8.01 (s, 1H), 7.86-7.76 (m, 2H), 5.70-5.78 (d, J=8 Hz, 1H), 5.50 (s, 2H), 5.15-5.11 (m, 1H), 3.91-3.78 (m, 3H), 3.72-3.58 (m, 1H), 3.47 (s, 3H), 2.83-2.69 (m, 1H), 2.13-1.95 (m, 3H), 1.93-1.91 (d, J=6.8 Hz, 2H), 1.85-1.75 (m, 3H), 1.24-1.15 (m, 1H), 1.05-1.01 (m, 2H), 0.93-0.80 (m, 3H) ppm. MS: M/e 485 (M+1)⁺.

### Compound A65: Mixture of 2-(7-((2S,5R)-2,5-diethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-diethyl-4-(1-(2-methylquinoxalin-6-yl)ethyl)piperazin-1-yl)-4-methyl-5-oxo-4,5-dihydro-2H-pyrazolo[4,3-b]pyridin-2-yl)acetonitrile

### Step A: mixture of 3-methylquinoxaline-6-carboxylic acid and 2-methylquinoxaline-6-carboxylic acid

To a solution of 3,4-diaminobenzoic acid (10 g, 65.72 mmol) and 2-oxopropanal (14.21 g, 40% in Water) in MeOH (100 mL) was stirred to room temperature for 4 hours. Then the mixture was concentrated under reduced pressure to give the crude product (90% purity) which was used directly for next step without purification. MS: M/e 189 (M+1)⁺.

### Step B: mixture of N-methoxy-N,3-dimethylquinoxaline-6-carboxamide and N-methoxy-N,2-dimethylquinoxaline-6-carboxamide

To a solution of 3-methylquinoxaline-6-carboxylic acid (12 g, 64.2 mmol), N,O-dimethylhydroxylamine (9.2 g, 96.26 mmol) and HATU (36.6 g, 96.26 mmol) in DMF (150 mL) was added NaHCO₃ (16.2 g, 192.6 mmol). The mixture solution was stirred at room temperature for 12 hours. Then the mixture was quenched with saturated NH₄Cl (60 mL) and extracted with EA (300 mL×2). The combined organic layers were washed with brine (50 mL×3), dried over Na₂SO₄ and concentrated under reduced pressure. The resulting residue was purified by flash column chromatography to give the titled compound (11 g, 74%). MS: M/e 232 (M+1)⁺.

### Step C: mixture of 1-(3-methylquinoxalin-6-yl)ethan-1-one and 1-(2-methylquinoxalin-6-yl)ethan-1-one

To a solution of N-methoxy-N,3-dimethylquinoxaline-6-carboxamide (8.6 g, 37.2 mmol) in anhydrous THF (100 mL) was degassed 3 times under N₂ atmosphere. The CH₃MgBr (18.6 mL, 55.8 mmol, 3M) was added dropwise at 0° C. The mixture was stirred to room temperature for 4 hours. Then the mixture was quenched with saturated NH₄Cl (60 mL) and extracted with EA (150 mL×3). The combined organic layers were washed with brine (50 mL×3), dried over Na₂SO₄ and concentrated under reduced pressure. The resulting residue was purified by flash column chromatography to give the titled compound (4.0 g, 57%). MS: M/e 187 (M+1)⁺.

### Step D: mixture of 1-(3-methylquinoxalin-6-yl)ethan-1-ol and 1-(2-methylquinoxalin-6-yl)ethan-1-ol

To a solution of 1-(3-methylquinoxalin-6-yl)ethan-1-one (1.15 g, 6.18 mmol) in MeOH (30 mL) at 0° C. was added NaBH₄ (188 mg, 4.94 mmol). The mixture was stirred at 0° C. for 2 hours. Then the mixture was quenched with saturated NH₄Cl (20 mL) and extracted with EA (45 mL×3). The combined organic layers were washed with brine (20 mL×3), dried over Na₂SO₄ and concentrated under reduced pressure. The resulting residue was purified by flash column chromatography to give the titled compound (600 mg, 52%). MS: M/e 189 (M+1)⁺.

### Step E: mixture of 7-((2S,5R)-2,5-diethyl-4-(1-(3-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one and 7-((2S,5R)-2,5-diethyl-4-(1-(2-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of 1-(3-methylquinoxalin-6-yl)ethan-1-ol (98 mg, 0.519 mmol), Intermediate 3 (150 mg, 0.432 mmol) and (cyanomethyl)trimethylphosphonium iodide (210 mg, 0.864 mmol) in CH₃CN (2 mL) was added DIPEA (279 mg, 2.16 mmol). The mixture solution was degassed 3 times under N₂ atmosphere. Then the mixture solution was stirred at 105° C. for 24 hours. The reaction mixture was quenched with saturated NH₄Cl (20 mL) at room temperature and extracted with EA (30 mL×2). The combined organic layers were dried over Na₂SO₄ and concentrated under reduced pressure. The resulting residue was purified by flash column chromatography to give the titled compound (110 mg, 47%). MS: M/e 544 (M+1)⁺.

### Step F: mixture of 7-((2S,5R)-2,5-diethyl-4-(1-(3-methylquinoxalin-6-yl)ethyl)piperazin-1-yl)-4-methyl-2,4-dihydro-5H-pyrazolo[4,3-b]pyridin-5-one and 7-((2S,5R)-2,5-diethyl-4-(1-(2-methylquinoxalin-6-yl)ethyl)piperazin-1-yl)-4-methyl-2,4-dihydro-5H-pyrazolo[4,3-b]pyridin-5-one

To a solution of 7-((2S,5R)-2,5-diethyl-4-(1-(3-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (100 mg, 0.184 mmol) in MeOH (5 mL) at room temperature was added HCl (3 mL, 4 M in 1,4-dioxane). The mixture was stirred at room temperature for 2 hours. Then the mixture was concentrated under reduced pressure to give the crude product (HCl salt). The crude product was basified by Na₂CO₃ (4M) to pH ˜10 and extracted with EA (35 mL×3). The combined organic layers were washed with brine (20 mL×3), dried over Na₂SO₄ and concentrated under reduced pressure to give the titled compound (80 mg, 94%). MS: M/e 460 (M+1)⁺.

### Step G: mixture of 2-(7-((2S,5R)-2,5-diethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-diethyl-4-(1-(2-methylquinoxalin-6-yl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-diethyl-4-(1-(3-methylquinoxalin-6-yl)ethyl)piperazin-1-yl)-4-methyl-2,4-dihydro-5H-pyrazolo[4,3-b]pyridin-5-one (80 mg, 0.174 mmol) and K₂CO₃ (72 mg, 0.522 mmol) in DMF (3 mL) at room temperature was added 2-iodoacetonitrile (44 mg, 0.261 mmol). The mixture solution was stirred at room temperature for 12 hours. Then the reaction mixture was quenched with saturated NaCl (20 mL) at room temperature and extracted with EA (30 mL×2). The combined organic layers were dried over Na₂SO₄ and concentrated under reduced pressure. The resulting residue was purified by Prep-TLC to give the titled compound (13 mg, 15%) as a mixture. ¹H NMR (400 MHz, CD₃OD) δ 8.81 (s, 1H), 8.07-7.93 (m, 4H), 5.56 (s, 1H), 5.47 (s, 2H), 4.04-3.88 (m, 1H), 3.63-3.58 (m, 1H), 3.31 (s, 3H), 3.10-2.77 (m, 2H), 2.76-2.63 (m, 4H), 2.61-2.38 (m, 1H), 2.17-1.88 (m, 2H), 1.88-1.61 (m, 2H), 1.45-1.41 (m, 3H), 1.60 (m, 1H), 1.43-0.98 (m, 3H), 0.56-0.42 (m, 3H) ppm. MS: M/e 499 (M+1)⁺.

### Compound A69: 2-(7-((2S,5R)-2,5-diethyl-4-(1-(2-fluoro-4-(trifluoromethyl)phenyl)ethyl)piperazin-1-yl)-4-methyl-5-oxo-4,5-dihydro-2H-pyrazolo[4,3-b]pyridin-2-yl)acetonitrile

### Step A: 1-(2-fluoro-4-(trifluoromethyl)phenyl)ethan-1-ol

To a solution of 1-(2-fluoro-4-(trifluoromethyl)phenyl)ethan-1-one (1.1 g, 5.3 mmol) in MeOH (20 mL) at 0° C. was added NaBH₄ (243 mg, 6.40 mmol). The mixture was stirred for 30 min. Then the mixture was quenched with saturated NH₄Cl (20 mL) and extracted with EA (30 mL×3). The combined organic layers were washed with brine (20 mL×3), dried over Na₂SO₄ and concentrated under reduced pressure. The resulting residue was purified by flash column chromatography to give the titled compound (900 mg, 82%). MS: M/e 191 (M−17)⁺.

### Step B: tert-butyl (2S,5R)-2,5-diethyl-4-(1-(2-fluoro-4-(trifluoromethyl)phenyl)ethyl)piperazine-1-carboxylate

To a solution of 1-(2-fluoro-4-(trifluoromethyl)phenyl)ethan-1-ol (1.0 g, 4.81 mmol), tert-butyl (2S,5R)-2,5-diethylpiperazine-1-carboxylate (1.17 g, 4.81 mmol) and (cyanomethyl)trimethylphosphonium iodide (1.17 g, 9.62 mmol) in CH₃CN (5 mL) was added DIPEA (3.1 g, 24.05 mmol). The mixture solution was degassed 3 times under N₂ atmosphere. Then the mixture solution was stirred at 105° C. for 24 hours. The reaction mixture was quenched with saturated NH₄Cl (20 mL) at room temperature and extracted with EA (30 mL×2). The combined organic layers were dried over Na₂SO₄ and concentrated under reduced pressure. The resulting residue was purified by flash column chromatography to give the titled compound (1.1 g, 53%). MS: M/e 433 (M+1)⁺.

### Step C: (2R,5S)-2,5-diethyl-1-(1-(2-fluoro-4-(trifluoromethyl)phenyl)ethyl)piperazine

To a solution of tert-butyl (2S,5R)-2,5-diethyl-4-(1-(2-fluoro-4-(trifluoromethyl)phenyl)ethyl)piperazine-1-carboxylate (1.0 g, 2.3 mmol) in DCM (20 mL) at room temperature was added HCl/dioxane (6 mL, 4M). The mixture was stirred at room temperature for 2 hours. Then the mixture was concentrated under reduced pressure to give the crude product (HCl salt). The crude product was basified by Na₂CO₃ (4M) to pH ˜10 and extracted with EA (30 mL×3). The combined organic layers were washed with brine (20 mL×3), dried over Na₂SO₄ and concentrated under reduced pressure to give the titled compound (700 mg, 91%) which was used directly for next step without purification. MS: M/e 333 (M+1)⁺.

### Step D: 7-((2S,5R)-2,5-diethyl-4-(1-(2-fluoro-4-(trifluoromethyl)phenyl)ethyl)piperazin-1-yl)-4-methyl-2-(tetrahydro-2H-pyran-2-yl)-2,4-dihydro-5H-pyrazolo[4,3-b]pyridin-5-one

To a solution of (2R,5S)-2,5-diethyl-1-(1-(2-fluoro-4-(trifluoromethyl)phenyl)ethyl)piperazine (700 mg, 2.1 mmol) and Intermediate 2 (953 mg, 2.5 mmol) in CH₃CN (2 mL) at room temperature was added DIPEA (1354 mg, 10.5 mmol). The mixture was stirred at 105° C. for 24 hours. Then the mixture was concentrated under reduced pressure to give the crude product. The crude product was purified by flash column chromatography to give the titled compound (1.0 g, 85%). MS: M/e 564 (M+1)⁺.

### Step E: 7-((2S,5R)-2,5-diethyl-4-(1-(2-fluoro-4-(trifluoromethyl)phenyl)ethyl)piperazin-1-yl)-4-methyl-2,4-dihydro-5H-pyrazolo[4,3-b]pyridin-5-one (Compound A231)

To a solution of 7-((2S,5R)-2,5-diethyl-4-(1-(2-fluoro-4-(trifluoromethyl)phenyl)ethyl)piperazin-1-yl)-4-methyl-2-(tetrahydro-2H-pyran-2-yl)-2,4-dihydro-5H-pyrazolo[4,3-b]pyridin-5-one (1.0 g, 1.77 mmol) in MeOH (20 mL) at room temperature was added HCL/dioxane (10 mL, 4M). The mixture was stirred at room temperature for 2 hours. Then the mixture was concentrated under reduced pressure to give the crude product (HCl salt). The crude product was basified by Na₂CO₃ (4M) to pH ˜10 and extracted with EA (35 mL×3). The combined organic layers were washed with brine (20 mL×3), dried over Na₂SO₄ and concentrated under reduced pressure to give the titled compound (700 mg, 82%) as a mixture of diastereomers, some of which was further separated into Compound A231a (10 mg) and Compound A231b (10 mg) by Prep-HPLC (Method A).

Compound A231a: ¹H NMR (400 MHz, CDCl₃) δ 7.76 (s, 1H), 7.45 (s, 1H), 7.41 (s, 1H), 7.27 (s, 1H), 5.65 (s, 1H), 4.05 (s, 1H), 3.48-3.42 (m, 4H), 3.09-2.99 (m, 1H), 2.75-2.65 (m, 1H), 2.32-2.22 (m, 1H), 1.87-1.55 (m, 5H), 1.50-1.46 (m, 1H), 1.29-1.25 (m, 3H), 0.93 (s, 3H), 0.61-0.56 (m, 3H) ppm. MS: M/e 480 (M+1)⁺.

Compound A231b: ¹H NMR (400 MHz, CDCl₃) δ 10.62 (s, 1H), 7.68-7.66 (t, J=8 Hz, 1H), 7.43 (s, 2H), 7.32-7.29 (m, 1H), 5.62 (s, 1H), 4.22-4.21 (m, 1H), 3.45 (s, 3H), 3.22 (m, 1H), 2.91 (m, 2H), 2.36-2.34 (m, 1H), 2.05-2.00 (m, 1H), 1.78-1.72 (m, 1H), 1.62-1.56 (m, 3H), 1.37-1.31 (m, 1H), 1.29-1.23 (m, 3H), 0.90-0.86 (t, J=8 Hz, 3H), 0.71-0.67 (t, J=8 Hz, 3H) ppm. MS: M/e 480 (M+1)⁺.

### Step F: 2-(7-((2S,5R)-2,5-diethyl-4-(1-(2-fluoro-4-(trifluoromethyl)phenyl)ethyl)piperazin-1-yl)-4-methyl-5-oxo-4,5-dihydro-2H-pyrazolo[4,3-b]pyridin-2-yl)acetonitrile (Compound A69)

To a solution of 7-((2S,5R)-2,5-diethyl-4-(1-(2-fluoro-4-(trifluoromethyl)phenyl)ethyl)piperazin-1-yl)-4-methyl-2,4-dihydro-5H-pyrazolo[4,3-b]pyridin-5-one (700 mg, 1.46 mmol) and K₂CO₃ (403 mg, 2.92 mmol) in DMF (20 mL) at room temperature was added 2-iodoacetonitrile (366 mg, 2.19 mmol). The mixture solution was stirred at room temperature for 12 hours. Then the reaction mixture was quenched with saturated NaCl (20 mL) at room temperature and extracted with EA (50 mL×2). The combined organic layers were dried over Na₂SO₄ and concentrated under reduced pressure to give crude Compound A69 (600 mg) as a mixture of diastereomers, which was further separated into Compound A69a (106 mg) and Compound A69b (150 mg) by Prep-HPLC (Method A).

Compound A69a (the earlier peak): ¹H NMR (400 MHz, CD₃OD) δ 7.92-7.91 (m, 1H), 7.80 (t, J=7.2 Hz, 1H), 7.53 (d, J=8 Hz, 1H), 7.44 (d, J=10 Hz, 1H), 5.56 (s, 1H), 5.47 (s, 2H), 4.28-4.23 (m, 1H), 3.43 (s, 3H), 3.32-3.30 (m, 3H), 2.92-2.88 (m, 2H), 2.40-2.38 (m, 1H), 2.11-2.07 (m, 1H), 1.80-1.76 (m, 1H), 1.56-1.52 (m, 2H), 1.36-1.32 (m, 3H), 0.95-0.91 (t, J=8 Hz, 3H), 0.78-0.74 (t, J=8 Hz, 3H) ppm. MS: M/e 519 (M+1)⁺.

Compound A69b (the later peak): ¹H NMR (400 MHz, CD₃OD) δ 7.95-7.91 (m, 1H), 7.87 (t, J=7.2 Hz, 1H), 7.51 (d, J=8 Hz, 1H), 7.41 (d, J=10.4 Hz, 1H), 5.56 (s, 1H), 5.48 (s, 2H), 4.10-4.07 (m, 1H), 3.52-3.51 (m, 1H), 3.43 (s, 3H), 3.31 (s, 2H), 3.18-3.14 (m, 1H), 2.76-2.65 (m, 1H), 2.39-2.29 (m, 1H), 1.95-1.91 (m, 1H), 1.70-1.66 (m, 2H), 1.55-1.51 (m, 1H), 1.36-1.32 (m, 3H), 1.03-0.99 (t, J=8 Hz, 3H), 0.67-0.63 (t, J=8 Hz, 3H) ppm. MS: M/e 519 (M+1)⁺.

### Compound A71: 2-(7-((2S,5R)-2,5-diethyl-4-(1-(4-fluoro-2-methoxyphenyl)ethyl)piperazin-1-yl)-4-methyl-5-oxo-4,5-dihydro-2H-pyrazolo[4,3-b]pyridin-2-yl)acetonitrile

### Step A: tert-butyl (2S,5R)-2,5-diethyl-4-(1-(4-fluoro-2-methoxyphenyl)ethyl)piperazine-1-carboxylate

A mixture of tert-butyl (2S,5R)-2,5-diethylpiperazine-1-carboxylate (1.5 g, 6.2 mmol), 1-(4-fluoro-2-methoxyphenyl)ethan-1-ol (1.58 g, 9.3 mmol), (cyanomethyl)trimethylphosphonium iodide (3.0 g, 12.4 mmol) and DIPEA (3.96 g, 31 mmol) in CH₃CN (10 mL) was heated to 105° C. for overnight under N₂ atmosphere. The solvent was removed under vacuum. The crude product was purified by silica flash column chromatography (DCM:MeOH=100:1) to give the titled compound (2.1 g, 86%). MS: M/e 395 (M+1)⁺.

### Step B: (2R,5S)-2,5-diethyl-1-(1-(4-fluoro-2-methoxyphenyl)ethyl)piperazine

To a solution of tert-butyl (2S,5R)-2,5-diethyl-4-(1-(4-fluoro-2-methoxyphenyl)ethyl)piperazine-1-carboxylate (2.1 g, 5.3 mmol) in DCM (20 mL) were added TFA (4 mL) at room temperature. The resulting mixture was stirred at room temperature for another 3 hours. The reaction solvent was removed under vacuum. The crude product was dissolved with DCM (mL) and neutralized with aq·NaOH to pH=8. The organic layer was concentrated to give crude product (1.6 g), which was used in the next step directly without further purification. MS: M/e 295 (M+1)⁺.

### Step C: 7-((2S,5R)-2,5-diethyl-4-(1-(4-fluoro-2-methoxyphenyl)ethyl)piperazin-1-yl)-4-methyl-2-(tetrahydro-2H-pyran-2-yl)-2,4-dihydro-5H-pyrazolo[4,3-b]pyridin-5-one

A mixture of Intermediate 2 (773 mg, 2.03 mmol), (2R,5S)-2,5-diethyl-1-(1-(4-fluoro-2-methoxyphenyl)ethyl)piperazine (600 mg, 2.03 mmol) and DIPEA (mg, mmol) in CH₃CN was heated to 105° C. for overnight under N₂ atmosphere. The solvent was removed under vacuum. The crude product was purified by flash column chromatography (DCM:MeOH=20:1) to give the titled compound (750 mg, 71%). MS: M/e 526 (M+1)⁺.

### Step D: 7-((2S,5R)-2,5-diethyl-4-(1-(4-fluoro-2-methoxyphenyl)ethyl)piperazin-1-yl)-4-methyl-2,4-dihydro-5H-pyrazolo[4,3-b]pyridin-5-one

To a solution of 7-((2S,5R)-2,5-diethyl-4-(1-(4-fluoro-2-methoxyphenyl)ethyl)piperazin-1-yl)-4-methyl-2-(tetrahydro-2H-pyran-2-yl)-2,4-dihydro-5H-pyrazolo[4,3-b]pyridin-5-one (400 mg, 0.76 mmol) in MeOH (5 mL) was added HCl dioxane solution (1.9 mL, 7.6 mmol, 4M) at room temperature. The resulting mixture was stirred at room temperature for another 2 hours. The reaction solvent was removed under vacuum. The crude product was dissolved with DCM (20 mL) and neutralized with aq·NaOH to pH=8. The organic layer was concentrated to give crude product which further purified by column flash column chromatography (DCM:MeOH=10:1) to give the titled compound (280 mg, 84%). MS: M/e 442 (M+1)⁺.

### Step E: 2-(7-((2S,5R)-2,5-diethyl-4-(1-(4-fluoro-2-methoxyphenyl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-diethyl-4-(1-(4-fluoro-2-methoxyphenyl)ethyl)piperazin-1-yl)-4-methyl-2,4-dihydro-5H-pyrazolo[4,3-b]pyridin-5-one (280 mg, 0.64 mmol) and K₂CO₃ (178 mg, 12.8 mmol) in DMF (5 mL) were added 2-iodoacetonitrile (160 mg, 0.96 mmol). The resulting mixture was stirred at room temperature overnight. The reaction mixture was diluted with EA (20 mL) and washed with brine (20 mL×3). The organic layers were concentrated under reduced pressure, to obtain Compound A71. The resulting residue containing Compound A71 was purified by Prep-TLC (DCM:MeOH=15:1) to give two compounds Compound A71a (38 mg, 12%) and Compound A71b (crude). The Compound A71b (crude) was further purified by Prep-HPLC (Method A) to give Compound A71b (19 mg, 6%).

Compound A71a: ¹H NMR (400 MHz, CD₃OD) δ 7.92 (s, 1H), 7.55 (s, 1H), 6.74-6.66 (m, 2H), 5.54 (s, 1H), 5.47 (s, 2H), 4.10 (br s, 1H), 3.82 (s, 3H), 3.43 (s, 3H), 3.12-3.01 (m, 2H), 2.68-2.55 (m, 2H), 2.39-2.5 (m, 1H), 2.01 (br s, 1H), 1.65-1.48 (m, 4H), 1.35-1.21 (m, 3H), 1.05-0.98 (m, 3H), 0.72-0.68 (m, 3H) ppm. MS: M/e 481 (M+1)⁺.

Compound A71b: ¹H NMR (400 MHz, CD₃OD) δ 7.90 (s, 1H), 7.45 (s, 1H), 6.76-6.69 (m, 2H), 5.52 (s, 1H), 5.44 (s, 2H), 4.31 (br s, 1H), 3.81 (s, 3H), 3.41 (s, 3H), 3.35-3.23 (m, 1H), 2.93-2.63 (m, 4H), 2.45 (br s, 1H), 2.02 (br s, 1H), 1.78 (br s, 1H), 1.52-1.48 (m, 2H), 1.30-1.25 (m, 3H), 0.99-0.92 (m, 3H), 0.75-0.68 (m, 3H) ppm. MS: M/e 481 (M+1)⁺.

### Compound A79: 2-(7-((2S,5R)-2,5-diethyl-4-(1-(4-fluoro-2-(methoxymethyl)phenyl)ethyl)piperazin-1-yl)-4-methyl-5-oxo-4,5-dihydro-2H-pyrazolo[4,3-b]pyridin-2-yl)acetonitrile

### Step A: tert-butyl (2S,5R)-2,5-diethyl-4-(1-(4-fluoro-2-(methoxymethyl)phenyl)ethyl)piperazine-1-carboxylate

To a solution of 1-(4-fluoro-2-(methoxymethyl)phenyl)ethan-1-ol (200 mg, 1.1 mmol) in MeCN (10 mL) was added tert-butyl (2S,5R)-2,5-diethylpiperazine-1-carboxylate (290 mg, 1.2 mmol), (cyanomethyl)trimethylphosphonium iodide (550 mg, 2.3 mmol) and DIPEA (700 mg, 5.4 mmol). The resulting mixture was stirred at 100° C. overnight. The reaction mixture was concentrated and purified by reversed C18 column (water, 0.05% TFA/MeCN) to give the titled compound (140 mg, 32%). MS: M/e 409 (M+1)⁺.

### Step B: (2R,5S)-2,5-diethyl-1-(1-(4-fluoro-2-(methoxymethyl)phenyl)ethyl)piperazine

To a solution of tert-butyl (2S,5R)-2,5-diethyl-4-(1-(4-fluoro-2-(methoxymethyl)phenyl)ethyl)piperazin (140 mg, 0.34 mmol) in DCM (5 mL) were added TFA (2 mL). The resulting mixture was stirred at RT for another 3 hours. The reaction solvent was removed under vacuum. The crude product was dissolved with water and adjusted to pH=9 with saturated NaHCO₃ aq. The aqueous layer was extracted with EA (25 mL×2). The organic layers were dried with Na₂SO₄, concentrated to afford crude product. The crude product was purified by Prep-TLC (DCM:MeOH=50:1) to give the titled compound (80 mg, 76%). MS: M/e 309 (M+1)⁺.

### Step C: 7-((2S,5R)-2,5-diethyl-4-(1-(4-fluoro-2-(methoxymethyl)phenyl)ethyl)piperazin-1-yl)-4-methyl-2-(tetrahydro-2H-pyran-2-yl)-2,4-dihydro-5H-pyrazolo[4,3-b]pyridin-5-one

To a solution of (2R,5S)-2,5-diethyl-1-(1-(4-fluoro-2-(methoxymethyl)phenyl)ethyl)piperazine (80 mg, 0.26 mmol) in MeCN (5 mL) and was added Intermediate 2 (148 mg, 0.39 mmol) and DIPEA (100 mg, 0.78 mmol). The resulting mixture was stirred at 100° C. overnight. The reaction solvent was removed under reduce pressure to afford crude product. The crude product was purified by Prep-TLC (DCM:MeOH=40:1) to give the titled compound (130 mg, 93%). MS: M/e 540 (M+1)⁺.

### Step D: 7-((2S,5R)-2,5-diethyl-4-(1-(4-fluoro-2-(methoxymethyl)phenyl)ethyl)piperazin-1-yl)-4-methyl-2,4-dihydro-5H-pyrazolo[4,3-b]pyridin-5-one

To a solution of 7-((2S,5R)-2,5-diethyl-4-(1-(4-fluoro-2-(methoxymethyl)phenyl)ethyl)piperazin-1-yl)-4-methyl-2-(tetrahydro-2H-pyran-2-yl)-2,4-dihydro-5H-pyrazolo[4,3-b]pyridin-5-one (130 mg, 0.24 mmol) in DCM (5 mL) was added TFA (2 mL). The resulting mixture was stirred at RT for 2 hours. The reaction solvent was removed under vacuum. The crude product was dissolved with water and adjusted to pH=9 with sat. NaHCO₃ aq. The aqueous layer was extracted with (DCM:i-PrOH=4:1, 25 mL×2). The organic layers were dried with Na₂SO₄, concentrated to afford crude product. The crude product was purified by Prep-TLC (DCM:MeOH=30:1) to give the titled compound (65 mg, 59%). MS: M/e 456 (M+1)⁺.

### Step E: 2-(7-((2S,5R)-2,5-diethyl-4-(1-(4-fluoro-2-(methoxymethyl)phenyl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-diethyl-4-(1-(4-fluoro-2-(methoxymethyl)phenyl)ethyl)piperazin-1-yl)-4-methyl-2,4-dihydro-5H-pyrazolo[4,3-b]pyridin-5-one (20 mg, 0.044 mmol) in DMF (2 mL) were added K₂CO₃ (18 mg, 0.13 mmol) and 2-iodoacetonitrile (11 mg, 0.065 mmol). The resulting mixture was stirred at RT overnight. The reaction mixture was diluted with EA (20 mL) and washed with water. The organic layers were dried with Na₂SO₄, concentrated to afford crude product. The crude product was purified by Prep-TLC (DCM:MeOH=20:1) to give the titled compound (7.8 mg, 36%). ¹H NMR (400 MHz, CD₃OD) δ 7.92 (s, 1H), 7.75-7.51 (m, 1H), 7.23-6.95 (m, 2H), 5.56 (s, 1H), 5.47 (s, 2H), 4.68-4.48 (m, 2H), 4.15-3.80 (m, 1H), 3.55-3.30 (m, 7H), 3.25-3.01 (m, 1H), 2.95-2.70 (m, 1H), 2.65-2.30 (m, 1H), 2.29-2.02 (m, 1H), 2.00-1.75 (m, 1H), 1.74-1.33 (m, 3H), 1.32-1.20 (m, 4H), 1.15-0.80 (m, 3H), 0.75-0.51 (m, 3H) ppm. MS: M/e 495 (M+1)⁺.

### Compound A84: 2-(7-((2S,5R)-2,5-diethyl-4-(1-(4-fluoro-2-methylphenyl)ethyl)piperazin-1-yl)-4-methyl-5-oxo-4,5-dihydro-2H-pyrazolo[4,3-b]pyridin-2-yl)acetonitrile

### Step A: 7-((2S,5R)-2,5-diethyl-4-(1-(4-fluoro-2-methylphenyl)ethyl)piperazin-1-yl)-4-methyl-2-(tetrahydro-2H-pyran-2-yl)-2,4-dihydro-5H-pyrazolo[4,3-b]pyridin-5-one

A mixture of Intermediate 3 (100 mg, 0.27 mmol), 1-(4-fluoro-2-methylphenyl)ethan-1-ol (83 mg, 0.54 mmol), (cyanomethyl)trimethylphosphonium iodide (131 mg, 0.54 mmol) and DIPEA (350 mg, 2.7 mmol) in CH₃CN (2 mL) was heated to 105° C. for overnight under N₂ atmosphere. The solvent was removed under vacuum. The crude product was purified by pre-TLC (DCM:MeOH=15:1) to give the titled compound (80 mg, 59%). MS: M/e 510 (M+1)⁺.

### Step B: 7-((2S,5R)-2,5-diethyl-4-(1-(4-fluoro-2-methylphenyl)ethyl)piperazin-1-yl)-4-methyl-2,4-dihydro-5H-pyrazolo[4,3-b]pyridin-5-one

To a solution of 7-((2S,5R)-2,5-diethyl-4-(1-(4-fluoro-2-methylphenyl)ethyl)piperazin-1-yl)-4-methyl-2-(tetrahydro-2H-pyran-2-yl)-2,4-dihydro-5H-pyrazolo[4,3-b]pyridin-5-one (80 mg, 0.16 mmol) in MeOH (2 mL) was added HCl dioxane solution (1 mL, 4 mmol, 4M) at room temperature. The resulting mixture was stirred at room temperature for another 2 hours. The reaction solvent was removed under vacuum. The crude product was dissolved with DCM (10 mL) and neutralized with aq·NaOH to PH=8. The organic layer was concentrated to give crude product which further purified by Prep-TLC (DCM:MeOH=10:1) to give the titled compound (20 mg, 29%). MS: M/e 426 (M+1)⁺.

### Step C: 2-(7-((2S,5R)-2,5-diethyl-4-(1-(4-fluoro-2-methylphenyl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-diethyl-4-(1-(4-fluoro-2-methylphenyl)ethyl)piperazin-1-yl)-4-methyl-2,4-dihydro-5H-pyrazolo[4,3-b]pyridin-5-one (20 mg, 0.05 mmol) and K₂CO₃ (13 mg, 0.09 mmol) in DMF (2 mL) were added 2-iodoacetonitrile (12 mg, 0.07 mmol). The resulting mixture was stirred at room temperature overnight. The reaction mixture was diluted with EA (10 mL) and washed with brine (10 mL×3). The organic layers were concentrated under reduced pressure. The resulting residue was purified by Prep-TLC (DCM:MeOH=15:1) to give the titled compound (2.2 mg, 10%). ¹H NMR (400 MHz, CD₃OD) δ 7.92 (s, 1H), 7.58-7.45 (m, 1H), 6.91-6.81 (m, 2H), 5.55-5.47 (m, 3H), 3.97-3.87 (m, 1H), 3.51-3.42 (m, 4H), 3.19-3.02 (m, 1H), 2.82-2.62 (m, 3H), 2.47-2.29 (m, 4H), 1.75-1.61 (m, 3H), 1.35-1.24 (m, 5H), 1.55-0.88 (m, 3H), 0.69-0.63 (m, 2H) ppm. MS: M/e 465 (M+1)⁺.

### Compound A95: 2-(7-((2S,5R)-2,5-diethyl-4-(1-(quinoxalin-6-yl)ethyl)piperazin-1-yl)-4-(2-hydroxyethyl)-5-oxo-4,5-dihydro-2H-pyrazolo[4,3-b]pyridin-2-yl)acetonitrile

### Step A: ethyl 4-(N-(2-((tert-butyldimethylsilyl)oxy)ethyl)acetamido)-1-(tetrahydro-2H-pyran-2-yl)-1H-pyrazole-3-carboxylate

A mixture of ethyl 4-acetamido-1-(tetrahydro-2H-pyran-2-yl)-1H-pyrazole-3-carboxylate (1 g, 3.56 mmol), tert-butyl(2-iodoethoxy)dimethylsilane (1.53 g, 5.35 mmol) and Cs₂CO₃ (2.32 g, 7.12 mmol) in DMF (10 ml) was stirred at RT overnight and then 60° C. for 2 days. After completed, the mixture was poured into water (15 ml) and then extracted with EA (20 ml×2). The organic layer was washed with brine (10 ml), dried and concentrated under reduced pressure. The resulting residue was purified by flash column flash column chromatography with 0-50% EA in PE to give the titled compound (0.37 g, 24%). MS: M/e 440 (M+1)⁺.

### Step B: 4-(2-((tert-butyldimethylsilyl)oxy)ethyl)-7-hydroxy-2-(tetrahydro-2H-pyran-2-yl)-2,4-dihydro-5H-pyrazolo[4,3-b]pyridin-5-one

To a stirred solution of ethyl 4-(N-(2-((tert-butyldimethylsilyl)oxy)ethyl)acetamido)-1-(tetrahydro-2H-pyran-2-yl)-1H-pyrazole-3-carboxylate (0.37 g, 0.84 mmol) in THF (10 ml) at −60° C. under N₂, was added L1HMDS (1M, 1.6 ml, 1.6 mmol) dropwise. The solution was stirred at −60° C. for 1.5 hours. After completed, the solution was quenched with H₂O (1 ml) and warmed to RT. The reaction mixture was concentrated under reduced pressure. The resulting residue was purified by flash column flash column chromatography with 0-10% MeOH in DCM to give the titled compound (250 mg, 75%) as light yellow solid. MS: M/e 394 (M+1)⁺.

### Step C: 4-(2-((tert-butyldimethylsilyl)oxy)ethyl)-5-oxo-2-(tetrahydro-2H-pyran-2-yl)-4,5-dihydro-2H-pyrazolo[4,3-b]pyridin-7-yl trifluoromethanesulfonate

A mixture of 4-(2-((tert-butyldimethylsilyl)oxy)ethyl)-7-hydroxy-2-(tetrahydro-2H-pyran-2-yl)-2,4-dihydro-5H-pyrazolo[4,3-b]pyridin-5-one (250 mg, 0.63 mmol), 1,1,1-trifluoro-N-phenyl-N-((trifluoromethyl)sulfonyl)methanesulfonamide (340 mg, 0.95 mmol) and K₂CO₃ (175 mg, 1.27 mmol) in THF (10 ml) was stirred at RT overnight. After completed, the solution was diluted with EA (15 ml), washed with brine (10 ml), dried and concentrated under reduced pressure. The resulting residue was purified by flash column flash column chromatography with 0-30% EA in PE to give the titled compound (290 mg, 87%). MS: M/e 526 (M+1)⁺.

### Step D: 4-(2-((tert-butyldimethylsilyl)oxy)ethyl)-7-((2S,5R)-2,5-diethyl-4-(1-(quinoxalin-6-yl)ethyl)piperazin-1-yl)-2-(tetrahydro-2H-pyran-2-yl)-2,4-dihydro-5H-pyrazolo[4,3-b]pyridin-5-one

A mixture of 4-(2-((tert-butyldimethylsilyl)oxy)ethyl)-5-oxo-2-(tetrahydro-2H-pyran-2-yl)-4,5-dihydro-2H-pyrazolo[4,3-b]pyridin-7-yl trifluoromethanesulfonate (290 mg, 0.55 mmol), 6-(1-((2R,5S)-2,5-diethylpiperazin-1-yl)ethyl)quinoxaline (165 mg, 0.55 mmol) and DIPEA (214 mg, 1.66 mmol) in MeCN (6 ml) was stirred at 110° C. for 4 days. After completed, the solution was concentrated under reduced pressure. The resulting residue was purified by flash column flash column chromatography with 30%-100% EA in PE to give the titled compound (140 mg, 38%). MS: M/e 674 (M+1)⁺.

### Step E: 7-((2S,5R)-2,5-diethyl-4-(1-(quinoxalin-6-yl)ethyl)piperazin-1-yl)-4-(2-hydroxyethyl)-2,4-dihydro-5H-pyrazolo[4,3-b]pyridin-5-one

To a solution of 4-(2-((tert-butyldimethylsilyl)oxy)ethyl)-7-((2S,5R)-2,5-diethyl-4-(1-(quinoxalin-6-yl)ethyl)piperazin-1-yl)-2-(tetrahydro-2H-pyran-2-yl)-2,4-dihydro-5H-pyrazolo[4,3-b]pyridin-5-one (140 mg, 0.21 mmol) in MeOH (2 ml), was added HCL/dioxane (4M, 2 ml, 8 mmol) dropwise and then stirred at RT for 1 hours. After completed, the reaction mixture was concentrated under reduced pressure to afford titled compound (98 mg, 100%), which was used directly for the next step without further purification. MS: M/e 476 (M+1)⁺.

### Step F: 4-(2-((tert-butyldimethylsilyl)oxy)ethyl)-7-((2S,5R)-2,5-diethyl-4-(1-(quinoxalin-6-yl)ethyl)piperazin-1-yl)-2,4-dihydro-5H-pyrazolo[4,3-b]pyridin-5-one

To a solution of 7-((2S,5R)-2,5-diethyl-4-(1-(quinoxalin-6-yl)ethyl)piperazin-1-yl)-4-(2-hydroxyethyl)-2,4-dihydro-5H-pyrazolo[4,3-b]pyridin-5-one (98 mg, 0.21 mmol), DIPEA (81 mg, 0.63 mmol) and DMAP (cat.) in DCM (5 ml), was added TBSCl (55 mg, 0.36 mmol) and then stirred at RT overnight. After completed, the reaction mixture was diluted with DCM (10 ml), washed with brine (10 ml), dried and concentrated under reduced pressure. The resulting residue was purified by Prep-TLC with DCM:MeOH (20:1) to afford crude product (120 mg, 100%), which was used directly for the next step without further purification. MS: M/e 590 (M+1)⁺.

### Step G: 2-(4-(2-(tert-butyldimethylsilyloxy)ethyl)-7-((2S,5R)-2,5-diethyl-4-(1-(quinoxalin-6-yl)ethyl)piperazin-1-yl)-5-oxo-4,5-dihydro-2H-pyrazolo[4,3-b]pyridin-2-yl)acetonitrile

A mixture of the compound of Step F (60 mg, 0.10 mmol), 2-iodoacetonitrile (25.5 mg, 0.15 mol) and K₂CO₃ (28 mg, 0.20 mmol) in DMF (2 ml) was stirred at RT for 5 hours. After completed, the mixture was poured into water (15 ml) and then extracted with EA (20 ml×2). The organic layer was washed with brine (10 ml), dried and concentrated under reduced pressure to afford crude product (63 mg), which was used directly for the next step without further purification. MS: M/e 629 (M+1)⁺.

### Step H: 2-(7-((2S,5R)-2,5-diethyl-4-(1-(quinoxalin-6-yl)ethyl)piperazin-1-yl)-4-(2-hydroxyethyl)-5-oxo-4,5-dihydro-2H-pyrazolo[4,3-b]pyridin-2-yl)acetonitrile

A mixture of the compound of Step G (63 mg, 0.1 mmol) and TBAF (1M, 0.15 ml, 0.15 mmol) in THF (5 ml) was stirred at RT for 0.5 hours. After completed, the solution was concentrated under reduced pressure. The resulting residue was purified by Prep-TLC with DCM:MeOH (15:1) and then Prep-HPLC to afford the titled compound (5 mg). ¹H NMR (400 MHz, DMSO-d6) δ 8.98-8.89 (m, 2H), 8.15-8.02 (m, 2H), 8.00-7.92 (m, 2H), 5.61 (s, 2H), 5.39 (s, 1H), 4.82 (t, J=5.3 Hz, 1H), 4.05 (d, J=6.3 Hz, 0.5H), 3.90 (d, J=6.4 Hz, 0.5H), 3.85 (s, 2H), 3.58 (dd, J=14.0, 4.0 Hz, 2H), 3.49-3.35 (m, 1H), 3.33-3.28 (m, 1H), 3.14 (s, 1H), 2.99 (d, J=11.3 Hz, 1H), 2.83 (d, J=9.9 Hz, 0.5H), 2.62 (d, J=10.7 Hz, 0.5H), 2.32 (d, J=7.3 Hz, 0.5H), 2.25 (d, J=12.0 Hz, 0.5H), 2.16-1.86 (m, 1H), 1.73-1.46 (m, 3H), 1.37 (dd, J=14.1, 6.2 Hz, 3H), 0.97 (dd, J=26.9, 20.2 Hz, 3H), 0.57 (dd, J=33.3, 26.6 Hz, 3H) ppm. MS: M/e 515 (M+1)⁺.

### Compound A96: 2-(7-((2S,5R)-2,5-diethyl-4-(1-(quinoxalin-6-yl)ethyl)piperazin-1-yl)-4-(2-methoxyethyl)-5-oxo-4,5-dihydro-2H-pyrazolo[4,3-b]pyridin-2-yl)acetonitrile

A mixture of 2-(7-((2S,5R)-2,5-diethyl-4-(1-(quinoxalin-6-yl)ethyl)piperazin-1-yl)-4-(2-hydroxyethyl)-5-oxo-4,5-dihydro-2H-pyrazolo[4,3-b]pyridin-2-yl)acetonitrile (8 mg, 0.016 mmol), CH₃I (2.4 mg, 0.017 mmol) and K₂CO₃ (3.2 mg, 0.023 mmol) in DMF (0.5 ml) was stirred at RT overnight. After completed, the mixture was poured into water (10 ml) and then extracted with EA (10 ml×2). The organic layer was washed with brine (10 ml), dried and concentrated under reduced pressure. The resulting residue was purified by Prep-HPLC to afford the titled compound (0.57 mg, FA salt). ¹H NMR (400 MHz, CD₃OD) δ 8.91-8.86 (m, 2H), 8.65-8.41 (m, 1H), 8.14-7.98 (m, 4H), 5.83-5.70 (m, 1H), 5.57 (d, J=6.3 Hz, 1H), 4.08 (d, J=7.8 Hz, 0.5H), 4.04 (d, J=5.6 Hz, 2H), 3.92 (d, J=6.3 Hz, 0.5H), 3.82 (t, J=5.7 Hz, 2H), 3.58 (d, J=12.7 Hz, 0.5H), 3.40-3.33 (m, 0.5H), 3.29-3.19 (m, 3H), 3.11 (d, J=13.7 Hz, 1H), 2.86-2.65 (m, 3H), 2.46-2.35 (m, 1H), 2.25-1.98 (m, 1H), 1.62 (s, 2H), 1.45 (dd, J=10.8, 6.5 Hz, 3H), 1.38-1.25 (m, 2H), 1.11-0.96 (m, 3H), 0.72-0.53 (m, 3H) ppm. MS: M/e 529 (M+1)⁺.

### Compound A97: 2-(7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of Intermediate 6 (510 mg, 1.2 mmol) and K₂CO₃ (330 mg, 2.4 mmol) in DMF (10 mL) were added 2-iodoacetonitrile (300 mg, 1.8 mmol). The resulting mixture was stirred at room temperature overnight. The reaction mixture was diluted with EA (40 mL) and washed with brine (20 mL×3). The organic layers were concentrated under reduced pressure. The resulting residue was purified by Prep-TLC (DCM:MeOH=15:1) to give the crude product Compound A97. The crude product was purified by chiral separation to give Compound A97a (24 mg), Compound A97b (34 mg). The chiral separation conditions are shown below.

Compound A97a (the earlier peak): ¹H NMR (400 MHz, CD₃OD) δ 8.88 (d, J=3.2 Hz, 2H), 8.13-8.02 (m, 3H), 7.92 (s, 1H), 5.59 (s, 1H), 5.44 (s, 2H), 4.09-4.05 (m, 1H), 3.43 (s, 3H), 3.32-3.31 (m, 2H), 3.08-2.95 (m, 2H), 2.93 (br s, 1H), 2.44 (br s, 1H), 1.65-1.61 (m, 2H), 1.46-1.44 (m, 6H), 0.72 (t, J=7.4 Hz, 3H) ppm. MS: M/e 471.3 (M+1)⁺.

Compound A97b (the later peak): ¹H NMR (400 MHz, CD₃OD) δ 8.87 (d, J=4.4 Hz, 2H), 8.09-8.03 (m, 3H), 7.93 (s, 1H), 5.57 (s, 1H), 5.47 (s, 2H), 4.42 (br s, 1H), 3.94-3.92 (m 1H), 3.61-3.58 (m, 1H), 3.44 (s, 3H), 3.24-3.21 (m, 1H), 2.85-2.81 (m, 2H), 2.27-2.21 (m, 1H), 1.78-1.56 (m, 2H), 1.45 (d, J=6.6 Hz, 3H), 1.24 (d, J=6.5 Hz, 3H), 1.09 (t, J=7.4 Hz, 3H) ppm. MS: M/e 471.3 (M+1)⁺.

Compound A98: 2-(7-((2S,5R)-2-ethyl-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: methyl benzyl-D-alaninate

To a solution of methyl D-alaninate (20.0 g, 0.19 mol) in CH₃CN (200 mL) was added benzyl bromide (32.5 g, 0.19 mol) at 0° C. under N₂ atmosphere. The reaction mixture was stirred at room temperature overnight. The solvent was removed under reduced pressure, The resulting residue was dissolved in EA (500 mL), washed with water (500 mL×3). The organic layers were concentrated under reduced pressure. The resulting residue was purified by flash column chromatography (PE:EA=10:1) to give the titled compound (17 g, 46%). MS: M/e 194 (M+1)⁺.

### Step B: methyl N-benzyl-N—((S)-2-((tert-butoxycarbonyl)amino)butanoyl)-D-alaninate

To a solution of methyl (R)-2-(benzylamino)butanoate (17.0 g, 0.09 mol), (S)-2-((tert-butoxycarbonyl)amino)butanoic acid (26.8 g, 0.13 mol) and 4-Methylmorpholine (18.2 g, 0.18 mol) in DCM (500 mL) was added HATU (49.4 g, 0.13 mmol) at 0° C. The reaction mixture was stirred at room temperature overnight. The reaction mixture was quenched by water and washed with water (1500 mL×2). The organic layers were concentrated under reduced pressure and purified by flash column chromatography (PE:EA=10:1) to give the titled compound (25 g, 75%). MS: M/e 379 (M+1)⁺.

### Step C: (3S,6R)-1-benzyl-3-ethyl-6-methylpiperazine-2,5-dione

To a solution of methyl (R)-2-((S)—N-benzyl-2-((tert-butoxycarbonyl)amino)butanamido)butanoate (25 g, 66 mmol) in 1,4-dioxane (20 mL) was added HCl dioxane solution (100 mL, 4M) at room temperature. The resulting mixture was stirred at room temperature for another 2 hours. The solvent was removed under vacuum. The resulting residue was dissolved in H₂O and neutralized by aq. NaHCO₃ to adjusted to pH=9. The resulting mixture was stirred at room temperature for 2 hours, then extracted with EA (500 mL×2). The organic layers were concentrated under reduced pressure and purified by flash column chromatography (PE:EA=10:1) to give the titled compound (15 g, 92%). MS: M/e 247 (M+1)⁺.

### Step D: (2R,5S)-1-benzyl-5-ethyl-2-methylpiperazine

To a solution of LiAlH4 (6.9 g, 0.18 mol) in THF (200 mL) was added slowly (3S,6R)-1-benzyl-3-ethyl-6-methylpiperazine-2,5-dione (15.0 g, 0.06 mol) which was dissolved in THF (100 mL) at 0° C. The resulting mixture was stirred at room temperature for 2 hours, then stirred at 80° C. for overnight. The reaction was quenched by water (7 mL) slowly at 0° C. Then, 1N aqueous NaOH solution (14 mL) and water (28 mL) was added sequentially. The resulting mixture was stirred for 2 hours. The white precipitates that formed was removed by filtration through Celite. The filter cake was washed with EA (500 mL). The combined filtrates were evaporated. The resulting residue was dissolved in toluene and taken to dryness to afford the titled compound (12 g, 91%). MS: M/e 219 (M+1)⁺.

### Step E: 7-((2S,5R)-4-benzyl-2-ethyl-5-methylpiperazin-1-yl)-4-methyl-2-(tetrahydro-2H-pyran-2-yl)-2,4-dihydro-5H-pyrazolo[4,3-b]pyridin-5-one

A mixture of Intermediate 2 (2.3 g, 6 mmol), (2R,5S)-1-benzyl-2,5-diethylpiperazine (2 g, 9 mmol) and DIPEA (3.9 g, 30 mmol) in CH₃CN (50 mL) was heated to 105° C. overnight under N₂ atmosphere. The solvent was removed under vacuum. The crude product was purified by flash column chromatography (DCM:MeOH=15:1) to give the titled compound (2.2 g, 82%). MS: M/e 450 (M+1)⁺.

### Step F: 7-((2S,5R)-2-ethyl-5-methylpiperazin-1-yl)-4-methyl-2-(tetrahydro-2H-pyran-2-yl)-2,4-dihydro-5H-pyrazolo[4,3-b]pyridin-5-one

A mixture of 7-((2S,5R)-4-benzyl-2-ethyl-5-methylpiperazin-1-yl)-4-methyl-2-(tetrahydro-2H-pyran-2-yl)-2,4-dihydro-5H-pyrazolo[4,3-b]pyridin-5-one (2.2 g, 4.9 mmol) and Pd/C (220 mg, 10% in water) in MeOH (30 mL) was shaken under N₂ atmosphere (50 psi) at room temperature for overnight. The mixture was filtered through Celite. The filter cake was washed with EA (50 mL). The combined filtrates were concentrated and purified by flash column chromatography (DCM:MeOH=10:1) to give the titled compound (1.3 g, 74%). MS: M/e 360 (M+1)⁺.

### Step G: 7-((2S,5R)-2-ethyl-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of 7-((2S,5R)-2-ethyl-5-methylpiperazin-1-yl)-4-methyl-2-(tetrahydro-2H-pyran-2-yl)-2,4-dihydro-5H-pyrazolo[4,3-b]pyridin-5-one (1.3 g, 3.6 mmol), 1-(quinoxalin-6-yl)ethan-1-ol (940 mg, 5.4 mmol), (cyanomethyl)trimethylphosphonium iodide (1.4 g, 5.4 mmol) and DIPEA (4.7 mg, 36 mmol) in CH₃CN (10 mL) was heated to 105° C. for overnight under N₂ atmosphere in a sealed tube. The solvent was removed under vacuum. The crude product was purified by flash column chromatography (DCM:MeOH=15:1) to give the titled compound (320 mg, 17%). MS: M/e 516 (M+1)⁺.

### Step H: 7-((2S,5R)-2-ethyl-5-methyl-4-(1-(quinoxalin-6-yl)ethyl)piperazin-1-yl)-4-methyl-2,4-dihydro-5H-pyrazolo[4,3-b]pyridin-5-one

To a solution of 7-((2S,5R)-2-ethyl-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (320 mg, 0.62 mmol) in MeOH (5 mL) was added HCl dioxane solution (10 mL, 40 mmol, 4M) at room temperature. The resulting mixture was stirred at room temperature for another 2 hours. The reaction solvent was removed under vacuum. The crude product was dissolved with DCM (10 mL) and neutralized with aq. NaOH to adjust pH=8. The organic layer was concentrated and purified by flash column chromatography (DCM:MeOH=15:1) to give the titled compound (150 mg, 56%). MS: M/e 432 (M+1)⁺.

### Step I: 2-(7-((2S,5R)-2-ethyl-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 7-((2S,5R)-2-ethyl-5-methyl-4-(1-(quinoxalin-6-yl)ethyl)piperazin-1-yl)-4-methyl-2,4-dihydro-5H-pyrazolo[4,3-b]pyridin-5-one (150 mg, 0.34 mmol) and K₂CO₃ (97 mg, 0.68 mmol) in DMF (2 mL) were added 2-iodoacetonitrile (85 mg, 0.51 mmol). The resulting mixture was stirred at room temperature overnight. The reaction mixture was diluted with EA (10 mL) and washed with brine (10 mL×3). The organic layers were concentrated under reduced pressure. The resulting residue was purified by Prep-TLC (DCM:MeOH=15:1) to give the crude product Compound A98. The crude product was purified by chiral separation to Compound A98a (27 mg) and Compound A98b (41 mg). The chiral separation conditions are shown below.

Compound A98a (the earlier peak): ¹H NMR (400 MHz, CD₃OD): δ 8.88 (d, J=3.6 Hz, 2H), 8.12-8.02 (m, 3H), 7.92 (s, 1H), 5.55 (s, 1H), 5.47 (s, 2H), 3.99-3.97 (m, 1H), 3.47 (br s, 1H), 3.43 (s, 3H), 3.33-3.31 (m, 2H), 3.09-2.89 (m, 3H), 2.17-2.15 (m, 1H), 1.92-1.90 (m, 1H), 1.44 (d, J=6.4 Hz, 3H), 1.03 (d, J=6.5 Hz, 3H), 0.96 (t, J=7.4 Hz, 3H) ppm. MS: M/e 471 (M+1)⁺.

Compound A98b (the later peak): ¹H NMR (400 MHz, CD₃OD): δ 8.87 (d, J=4.6 Hz, 2H), 8.10-8.01 (m, 3H), 7.92 (s, 1H), 5.56 (s, 1H), 5.46 (s, 2H), 3.83-3.81 (m, 1H), 3.68-3.65 (m, 2H), 3.43 (s, 3H), 3.33-3.31 (m, 2H), 2.79-2.77 (m, 1H), 2.35-2.32 (m, 1H), 1.98-1.96 (m, 1H), 1.72-1.70 (m, 1H), 1.45 (d, J=6.4 Hz, 3H), 1.19 (d, J=6.5 Hz, 3H), 0.58 (t, J=7.5 Hz, 3H) ppm. MS: M/e 471 (M+1)⁺.

### Compound A99: 2-(7-((2S,5S)-5-(hydroxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: methyl ((benzyloxy)carbonyl)-D-seryl-L-alaninate

To a stirred mixture of ((benzyloxy)carbonyl)-D-serine (11.95 g, 50 mmol) and methyl D-alaninate hydrochloride (7 g, 50 mmol) in CH₂Cl₂ (200 mL) was added HATU (22.9 g, 60 mmol), followed by DIPEA (19 g, 0.15 mol). After the addition, the reaction mixture was stirred overnight. The reaction mixture was washed with H₂O (200 mL). The organic layers were separated, washed with brine, dried over Na₂SO₄ and concentrated. The resulting residue was further washed with H₂O (200 mL) and filtered. The cake was collected, dried to give the titled compound (28 g, 86%). MS: M/e 325 (M+1)⁺.

### Step B: (3R,6S)-3-(hydroxymethyl)-6-methylpiperazine-2,5-dione

To a solution of methyl ((benzyloxy)carbonyl)-D-seryl-L-alaninate (16.2 g, 50 mmol) in MeOH (200 mL) was added Pd/C (2 g, 10% in water). Then the mixture was stirred overnight under H₂ (1 atm). The reaction mixture was filtered and the filtrate was concentrated to ˜100 mL. The resulting mixture was stirred at 100° C. for 2 days in a sealed tube. The reaction mixture was cooled to room temperature and MTBE (200 mL) was added and filtered. The cake was collected, dried to give the titled compound (3.5 g, 44.3%). MS: M/e 159 (M+1)⁺.

### Step C: ((2S,5S)-5-methylpiperazin-2-yl)methanol hydrochloride

A mixture of (3R,6S)-3-(hydroxymethyl)-6-methylpiperazine-2,5-dione (3.5 g, 22.2 mmol) in THF—BH₃ (1.0 M, 140 mL) was stirred at 70° C. overnight. MeOH (20 mL) was gradually added to quench the reaction at 0° C., followed by conc. HCl (10 mL). The resulting solid was filtered, the cake was collected, dried to give the titled compound (4.5 g, 100%). MS: M/e 131 (M+1)⁺.

### Step D: tert-butyl (2S,5S)-5-(hydroxymethyl)-2-methylpiperazine-1-carboxylate

To a stirred solution of ((2S,5S)-5-methylpiperazin-2-yl)methanol hydrochloride (4.5 g, 22.2 mmol) and Et₃N (8.9 g, 88.8 mmol) in MeOH (100 mL) was added dropwise a solution of Boc₂O (14.5 g, 66.6 mmol) in MeOH (10 mL) at 0-5° C. After then, the reaction mixture was stirred at 50° C. overnight. The reaction mixture was concentrated to give the residue, which was treated with EA/H₂O (100 mL/100 mL). The organic layer was separated, washed with brine, dried over Na₂SO₄ and purified by flash column chromatography (petroleum ether/EA=100/1˜1/1) to give di-tert-butyl (2S,5S)-2-(hydroxymethyl)-5-methylpiperazine-1,4-dicarboxylate (7.5 g, 100%), which was dissolved in EtOH/NaOH aq. (50 mL/50 mL) and the mixture was stirred at 80° C. for 2 days. Most EtOH was removed to give the aqueous layer which was acidified to pH=9-10 with HCl aq. (3 M), then extracted with EA (40 mL×10). The combined organic layers were washed with brine, dried over Na₂SO₄, concentrated to give the titled compound (4.6 g, 91%). MS: M/e 231 (M+1)⁺.

### Step E: tert-butyl (2S,5S)-5-(((tert-butyldimethylsilyl)oxy)methyl)-2-methylpiperazine-1-carboxylate

To a stirred solution of tert-butyl (2S,5S)-5-(hydroxymethyl)-2-methylpiperazine-1-carboxylate (2.12 g, 9.2 mmol) in CH₂Cl₂ (50 mL) was added Et₃N (1.86 g, 18.4 mmol), followed by DMAP (114 mg, 0.92 mmol), then TBSCl (2.08 g, 13.8 mmol). After the addition, the reaction mixture was stirred at room temperature overnight. The reaction mixture was treated with H₂O, extracted with CH₂Cl₂ (50 mL×2). The combined organic layers were washed with brine, dried over Na₂SO₄ and concentrated to dryness. The resulting residue was purified by flash column chromatography (PE:EA=2:1) to give the titled compound (2.72 g, 86%). MS: M/e 345 (M+1)⁺.

### Step F: tert-butyl (2S,5S)-5-(((tert-butyldimethylsilyl)oxy)methyl)-2-methyl-4-(1-(quinoxalin-6-yl)ethyl)piperazine-1-carboxylate

A mixture of tert-butyl (2S,5S)-5-(((tert-butyldimethylsilyl)oxy)methyl)-2-methylpiperazine-1-carboxylate (172 mg, 0.5 mmol), 1-(quinoxalin-6-yl)ethan-1-ol (174 mg, 1 mmol), (cyanomethyl)trimethylphosphonium iodide (364.5 mg, 1.5 mmol) and DIPEA (645 mg, 5 mmol) in CH₃CN (10 mL) was stirred at 100° C. overnight in a sealed tube. The reaction mixture was poured into H₂O (50 mL), extracted with EA (50 mL×2). The combined organic layers were washed with brine, dried over Na₂SO₄ and concentrated to dryness. The resulting residue was purified by Pre-TLC (EA) to give the titled compound (130 mg, 52%). MS: M/e 501 (M+1)⁺.

### Step G: ((2S,5S)-5-methyl-1-(1-(quinoxalin-6-yl)ethyl)piperazin-2-yl)methanol

To a stirred solution of tert-butyl (2S,5S)-5-(((tert-butyldimethylsilyl)oxy)methyl)-2-methyl-4-(1-(quinoxalin-6-yl)ethyl)piperazine-1-carboxylate (130 mg, 0.26 mmol) in CH₂Cl₂ (5 mL) was added HCl (2 mL, 4.0 M in 1,4-dioxane) and the mixture was stirred for 3 hours. The reaction mixture was concentrated to give the residue, which was used directly for next step without further purification. MS: M/e 287 (M+1)⁺.

### Step H: 7-((2S,5S)-5-(hydroxymethyl)-2-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of Intermediate 2 (33 mg, 0.26 mmol), ((2S,5S)-5-methyl-1-(1-(quinoxalin-6-yl)ethyl)piperazin-2-yl)methanol (0.26 mmol) and DIPEA (100.6 mg, 0.78 mmol) in CH₃CN (5 mL) was stirred at 75° C. for 3 days. The reaction mixture was poured into H₂O (10 mL), extracted with EA (10 mL×3). The combined organic layers were washed with brine, dried over Na₂SO₄ and concentrated to dryness. The resulting residue was purified by Pre-TLC (CH₂Cl₂/MeOH=10/1) to give the titled compound (13 mg). MS: M/e 518 (M+1)⁺.

### Step I: 7-((2S,5S)-5-(hydroxymethyl)-2-methyl-4-(1-(quinoxalin-6-yl)ethyl)piperazin-1-yl)-4-methyl-2,4-dihydro-5H-pyrazolo[4,3-b]pyridin-5-one

To a stirred solution of 7-((2S,5S)-5-(hydroxymethyl)-2-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (87 mg, 0.168 mmol) in MeOH/CH₂Cl₂ (5 mL/3 mL) was added HCl (2 mL, 4.0 M in 1,4-dioxane) and the mixture was stirred overnight. The reaction mixture was concentrated to give the residue, which was treated with saturated NaHCO₃ aq., then extracted with EA (10 mL×3). The combined organic layers were washed with brine, dried over Na₂SO₄ and concentrated to dryness. The resulting residue was purified by Pre-TLC (CH₂Cl₂/MeOH=10/1) to give the titled compound (54 mg, 74%). MS: M/e 434 (M+1)⁺.

### Step J: 2-(7-((2S,5S)-5-(hydroxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

A mixture of 7-((2S,5S)-5-(hydroxymethyl)-2-methyl-4-(1-(quinoxalin-6-yl)ethyl)piperazin-1-yl)-4-methyl-2,4-dihydro-5H-pyrazolo[4,3-b]pyridin-5-one (54 mg, 0.124 mmol), 2-iodoacetonitrile (42 mg, 0.25 mmol) and K₂CO₃ (34.5 g, 0.25 mmol) in DMF (4 mL) was stirred overnight. The reaction mixture was poured into H₂O (15 mL), extracted with EA (10 mL×3). The combined organic layers were washed with brine, dried over Na₂SO₄ and concentrated to dryness. The resulting residue was purified by Pre-TLC (CH₂Cl₂/MeOH=10/1) to give the titled compound (30 mg, 51%). ¹H NMR (400 MHz, DMSO-d₆) δ 8.93 (s, 2H), 8.19-7.90 (m, 4H), 5.62 (d, J=16.8 Hz, 2H), 5.45 (d, J=18 Hz, 1H), 5.08-4.53 (m, 1H), 4.48-4.05 (m, 2H), 3.82-3.38 (m, 3H), 3.28 (s, 3H), 3.19 (d, J=11.6 Hz, 0.5H), 2.98 (d, J=10.8 Hz, 1H), 2.89-2.62 (m, 2H), 2.24 (d, J=11.2 Hz, 0.5H), 1.52-1.35 (m, 3H), 1.23 (s, 1.5H), 1.06 (s, 1.5H) ppm. MS: M/e 473 (M+1)⁺.

### Compound A100: 2-(7-((2S,5S)-5-(methoxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: 2-(7-((2S,5S)-5-(chloromethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a stirred solution of 2-(7-((2S,5S)-5-(hydroxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile (23 mg, 0.05 mmol) in CH₂Cl₂ (5 mL) was added Et₃N (10 mg, 0.1 mmol), followed by MeSO₂Cl (6.7 mg, 0.06 mmol). After the addition, the reaction mixture was stirred overnight. The reaction mixture was diluted with CH₂Cl₂ (10 mL). washed with saturated NaHCO₃ aq., brine, dried over Na₂SO₄, concentrated to give the titled compound, which was used directly for next step without further purification. MS: M/e 491 (M+1)⁺.

### Step B: 2-(7-((2S,5S)-5-(methoxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

A mixture of 2-(7-((2S,5S)-5-(chloromethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile (10 mg, 0.02 mmol) in MeOH (3 mL) was stirred at 50° C. for 3 days. The reaction mixture was concentrated to give the residue, which was purified by Prep-TLC (CH₂Cl₂/MeOH=10/1) to give the titled compound (3 mg). ¹H NMR (400 MHz, DMSO-d₆) δ 8.90-8.85 (m, 2H), 8.14-7.99 (m, 3H), 7.94 (s, 1H), 5.62-5.57 (m, 1H), 5.48 (s, 2H), 4.40-4.20 (m, 1H), 4.15-4.01 (m, 1H), 3.85-3.67 (m, 2H), 3.66-3.47 (m, 2H), 3.44 (s, 3H), 2.97-2.74 (m, 2H), 2.39 (d, J=10.0 Hz, 1H), 2.27-2.01 (m, 1H), 1.54 (d, J=6.4 Hz, 2H), 1.52-1.46 (m, 3H), 1.38 (d, J=6.4 Hz, 1H), 1.26-1.18 (m, 3H) ppm. MS: M/e 487 (M+1)⁺.

### Compound A101: 2-(7-((2R,5R)-2-(hydroxymethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: tert-butyl (2R,5R)-5-(hydroxymethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate

A mixture Intermediate 2 (1.143 g, 3 mmol), tert-butyl (2R,5R)-5-(hydroxymethyl)-2-methylpiperazine-1-carboxylate (0.83 g, 3.6 mmol) and DIPEA (774 mg, 6 mmol) in CH₃CN (15 mL) was stirred at 100° C. over a weekend in a sealed tube. The reaction mixture was poured into H₂O (50 mL) and extracted with EA (20 mL×3). The combined organic layers were washed with brine, dried over Na₂SO₄ and concentrated to dryness. The resulting residue was purified by flash column chromatography to give the titled compound (930 mg, 67%). MS: M/e 462 (M+1)⁺.

### Step B: 7-((2R,5R)-2-(hydroxymethyl)-5-methylpiperazin-1-yl)-4-methyl-2-(tetrahydro-2H-pyran-2-yl)-2,4-dihydro-5H-pyrazolo[4,3-b]pyridin-5-one

To a stirred solution of tert-butyl (2R,5R)-5-(hydroxymethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (400 mg, 0.87 mmol) in CH₂Cl₂ (10 mL) was added TFA (2 mL). After the addition, the reaction mixture was stirred for 4 hours. The reaction mixture was concentrated to give the residue, which was treated with NaHCO₃ aq., extracted with EA (10 mL). The organic layer was discarded and the aqueous layer was extracted with CH₂Cl₂/IPA (2/1, 10 mL×3). The combined organic layers were washed with brine, dried over Na₂SO₄, concentrated to give the titled compound (130 mg, 41%). MS: M/e 362 (M+1)⁺.

### Step C: 7-((2R,5R)-2-(((tert-butyldimethylsilyl)oxy)methyl)-5-methylpiperazin-1-yl)-4-methyl-2-(tetrahydro-2H-pyran-2-yl)-2,4-dihydro-5H-pyrazolo[4,3-b]pyridin-5-one

To a stirred solution of 7-((2R,5R)-2-(hydroxymethyl)-5-methylpiperazin-1-yl)-4-methyl-2-(tetrahydro-2H-pyran-2-yl)-2,4-dihydro-5H-pyrazolo[4,3-b]pyridin-5-one (130 mg, 0.36 mmol) in CH₂Cl₂ (10 mL) was added Et₃N (73 mg, 0.72 mmol), followed by DMAP (4.5 mg, 0.036 mmol). Then TBSCl (84.6 mg, 0.54 mmol) was added. After the addition, the reaction mixture was stirred overnight. The reaction mixture was washed with H₂O (10 mL), brine, dried over Na₂SO₄ and concentrated to dryness. The resulting residue was purified by Pre-TLC (CH₂Cl₂/MeOH=10/1) to give the titled compound (70 mg, 41%). MS: M/e 476 (M+1)⁺.

### Step D: 7-((2R,5R)-2-(((tert-butyldimethylsilyl)oxy)methyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of the 7-((2R,5R)-2-(((tert-butyldimethylsilyl)oxy)methyl)-5-methylpiperazin-1-yl)-4-methyl-2-(tetrahydro-2H-pyran-2-yl)-2,4-dihydro-5H-pyrazolo[4,3-b]pyridin-5-one (70 mg, 0.66yridine66neuinoxalin-6-yl)ethan-1-ol (76.7 mg, 0.44 mmol), (cyanomethyl)trimethylphosphonium iodide (107 mg, 0.44 mmol) and DIPEA (189 mg, 1.47 mmol) in CH₃CN (3 mL) was stirred at 100° C. overnight in a sealed tube. The reaction mixture was diluted with EA (10 mL), washed with brine, dried over Na₂SO₄ and concentrated to dryness. The resulting residue was purified by Pre-TLC (EA) to give the titled compound (50 mg, 54%). MS: M/e 632 (M+1)⁺.

### Step E: 2-(7-((2R,5R)-2-(hydroxymethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a stirred solution of 7-((2R,5R)-2-(((tert-butyldimethylsilyl)oxy)methyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (50 mg, 0.079 mmol) in MeOH (5 mL) was added HCl (2 mL, 4.0 M in 1,4-dioxane) and the mixture was stirred overnight. The reaction mixture was concentrated to give the residue, which was dissolved in DMF/H₂O (3 mL/2 mL) and K₂CO₃ (21.8 mg, 0.158 mmol) was added followed by 2-iodoacetonitrile (26.4 mg, 0.158 mmol), and then the mixture was stirred for 2 hours. The reaction mixture was poured into H₂O (10 mL), extracted with EA (20 mL×2). The combined organic layers were washed with brine, dried over Na₂SO₄, concentrated to dryness. The resulting residue was purified by Pre-TLC (CH₂Cl₂/MeOH=10/1) to give the titled compound (21 mg). ¹H NMR (400 MHz, CD₃OD) δ 8.91-8.85 (m, 2H), 8.14-7.97 (m, 3H), 7.96-7.91 (m, 1H), 5.68-5.59 (m, 1H), 5.50-5.43 (m, 2H), 4.17-4.09 (m, 0.5H), 4.03-3.64 (m, 4.5H), 3.50-3.39 (m, 4H), 3.06-2.46 (m, 3H), 1.46 (d, J=6.4 Hz, 3H), 1.20 (d, J=6.0 Hz, 1.5H), 1.05 (d, J=6.4 Hz, 1.5H) ppm. MS: M/e 473 (M+1)⁺.

### Compound A102: 2-(7-((2S,5R)-2,5-dimethyl-4-(1-(quinoxalin-6-yl)ethyl)piperazin-1-yl)-5-oxo-4,5-dihydro-2H-pyrazolo[4,3-b]pyridin-2-yl)acetonitrile

### Step A: 7-((2S,5R)-2,5-dimethyl-4-(1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-b]pyridin-5-one

To a solution of Intermediate 7 (177 mg, 0.36 mmol) and Intermediate 1 (108 mg, 0.4 mmol) in CH₃CN (5 mL) was added DIPEA (93 mg, 0.72 mmol). Then the mixture was heated at 90° C. under N₂ for 56 hours. The mixture was cooled to room temperature, diluted with water (50 mL), extracted with EA (60 mL×2). The combined organic layers were washed with brine, dried over Na₂SO₄, filtered and concentrated to dryness. The resulting residue was The resulting residue was purified by flash column chromatography to give titled compound (150 mg, 68%). MS: M/e 608 (M+1)⁺.

### Step B: 7-((2S,5R)-2,5-dimethyl-4-(1-(quinoxalin-6-yl)ethyl)piperazin-1-yl)-2,4-dihydro-5H-pyrazolo[4,3-b]pyridin-5-one

To a solution of 7-((2S,5R)-2,5-dimethyl-4-(1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-b]pyridin-5-one (150 mg, 0.247 mmol) in TFA (4 mL) was added trifluoromethanesulfonic acid (4 mL) was add. The resulting mixture was stirred at room temperature overnight. The mixture was quenched with ice-water, basified with saturated Na₂CO₃ solution to PH 7-8, extracted with DCM/IPA (6/1, 60 mL×3). The combined organic layers were washed with brine, dried over Na₂SO₄, filtered and concentrated to dryness. The resulting residue was The resulting residue was purified by Prep-TLC to give titled compound (15 mg, 15%). MS: M/e 404 (M+1)⁺.

### Step C: 2-(7-((2S,5R)-2,5-dimethyl-4-(1-(quinoxalin-6-yl)ethyl)piperazin-1-yl)-5-oxo-4,5-dihydro-2H-pyrazolo[4,3-b]pyridin-2-yl)acetonitrile

To a solution of 7-((2S,5R)-2,5-dimethyl-4-(1-(quinoxalin-6-yl)ethyl)piperazin-1-yl)-2,4-dihydro-5H-pyrazolo[4,3-b]pyridin-5-one (15 mg, 0.037 mmol) and K₂CO₃ (10 mg, 0.074 mmol) in DMF (2 mL) was added 2-iodoacetonitrile (7 mg, 0.041 mmol). The reaction was stirred at room temperature for 4 hours. The reaction mixture was diluted with water, extracted with EA (60 mL), washed with brine, dried over Na₂SO₄, filtered, and concentrated to dryness. The resulting residue was purified by flash column chromatography to give titled compound (1 mg, 6%). ¹HNMR (400 MHz, DMSO-d₆) δ 10.43 (s, 1H), 8.98-8.87 (m, 2H), 8.16-7.90 (m, 3H), 7.71 (s, 1H), 5.56 (s, 2H), 5.29-5.24 (m, 1H), 4.02-3.86 (m, 1H), 3.81-3.75 (m, 0.5H), 3.62-3.48 (m, 1.5H), 3.30-3.23 (m, 0.5H), 2.99-2.92 (m, 1H), 2.90-2.66 (m, 2H), 2.15-1.95 (m, 0.5H), 1.42-1.28 (m, 5H), 1.16-1.08 (m, 2H), 1.00-0.94 (m, 2H) ppm. MS: M/e 443 (M+1)⁺.

### Compound A103: 2-(7-((2S,5R)-2,5-diethyl-4-(1-(4-(trifluoromethyl)phenyl)ethyl)piperazin-1-yl)-4-methyl-5-oxo-4,5-dihydro-2H-pyrazolo[4,3-b]pyridin-2-yl)acetonitrile

### Step A: 7-((2S,5R)-2,5-diethyl-4-(1-(4-(trifluoromethyl)phenyl)ethyl)piperazin-1-yl)-4-methyl-2-(tetrahydro-2H-pyran-2-yl)-2,4-dihydro-5H-pyrazolo[4,3-b]pyridin-5-one

To a solution of Intermediate 3 (150 mg, 0.4 mmol), 1-(4-(trifluoromethyl)phenyl)ethan-1-ol (114 mg, 0.6 mmol) and (cyanomethyl)trimethylphosphonium iodide (194 mg, 0.8 mmol) in CH₃CN (5 mL) was added DIPEA (155 mg, 0.8 mmol). The mixture was sealed in a bottle and heated at 100° C. for 16 hours. Then the mixture was cooled to room temperature, diluted with water, extracted with EA (60 mL×2), washed with brine, dried over Na₂SO₄, filtered, and concentrated to dryness. The resulting residue was purified by Prep-TLC (DCM:MeOH=15:1) to give titled compound (100 mg, 45%). MS: M/e 546 (M+1)⁺.

### Step B: 7-((2S,5R)-2,5-diethyl-4-(1-(4-(trifluoromethyl)phenyl)ethyl)piperazin-1-yl)-4-methyl-2,4-dihydro-5H-pyrazolo[4,3-b]pyridin-5-one

To a solution of 7-((2S,5R)-2,5-diethyl-4-(1-(4-(trifluoromethyl)phenyl)ethyl)piperazin-1-yl)-4-methyl-2-(tetrahydro-2H-pyran-2-yl)-2,4-dihydro-5H-pyrazolo[4,3-b]pyridin-5-one (100 mg, 0.183 mmol) in DCM (3 mL) was added TFA (8 mL). The mixture was stirred at room temperature for 16 h. The mixture was concentrated to dryness. The resulting residue was basified with saturated NaHCO₃ solution, extracted with DCM/IPA (3/1, 60 mL×2), washed with brine, dried over Na₂SO₄, filtered, and concentrated to dryness.

The resulting residue was purified by Prep-TLC (DCM/MeOH=10/1) to give titled compound (50 mg, 59%). MS: M/e 462 (M+1)⁺.

### Step C: 2-(7-((2S,5R)-2,5-diethyl-4-(1-(4-(trifluoromethyl)phenyl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-diethyl-4-(1-(4-(trifluoromethyl)phenyl)ethyl)piperazin-1-yl)-4-methyl-2,4-dihydro-5H-pyrazolo[4,3-b]pyridin-5-one (50 mg, 0.11 mmol) and K₂CO₃ (30 mg, 0.22 mmol) in DMF (4 mL) was added 2-iodoacetonitrile (28 mg, 0.165 mmol). The reaction was stirred at room temperature overnight. The reaction mixture was diluted with water, extracted with EA (60 mL×2), washed with brine, dried over Na₂SO₄, filtered, and concentrated to dryness. The resulting residue was purified by Prep-TLC (DCM:MeOH=15:1) to give titled compound (20 mg, 40%). ¹HNMR (400 MHz, CD₃OD) δ 7.94-7.91 (m, 1H), 7.69-7.54 (m, 4H), 5.54 (s, 1H), 5.49-5.44 (m, 2H), 3.89-3.81 (m, 0.5H), 3.74-3.66 (m, 0.5H), 3.55-3.46 (m, 0.5H), 3.43 (s, 3H), 3.31-3.26 (m, 2.5H), 3.20-3.10 (m, 0.5H), 3.06-2.98 (m, 0.5H), 2.92-2.83 (m, 0.5H), 2.72-2.63 (m, 0.5H), 2.41-2.25 (m, 1H), 2.19-2.06 (m, 0.5H), 2.00-1.75 (m, 1H), 1.75-1.49 (m, 2.5H), 1.39-1.28 (m, 3H), 1.03 (t, J=7.6 Hz, 1.5H), 0.96 (t, J=7.2 Hz, 1.5H), 0.70 (t, J=7.6 Hz, 1.5H), 0.62 (t, J=7.6 Hz, 1.5H) ppm. MS: M/e 501 (M+1)⁺.

Another batch Compound A103 (575 mg) was prepared according to the procedures as above described that could be recognized by one skilled in the art. Then Compound A103 (575 mg) as a mixture was further separated into Compound A103a (145 mg) and Compound A103b (195 mg) by chiral Prep-HPLC. The chiral separation conditions are shown below.

| Column | CHIRALPAK IG | |
|---|---|---|
| Column Size | 2 cm × 25 cm, 5 um | |
| Mobile Phase A | Hex | |
| Mobile Phase B | EtOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A103a (the earlier peak): ¹H NMR (400 MHz, CD₃OD) δ 7.92 (s, 1H), 7.65 (d, J=7.6 Hz, 2H), 7.58 (d, J=7.6 Hz, 2H), 5.56 (s, 1H), 5.46 (s, 2H), 4.83-4.20 (m, 1H), 3.91-3.80 (m, 1H), 3.43 (s, 3H), 3.28 (s, 2H), 3.02 (d, J=11.6 Hz, 1H), 2.88 (d, J=11.6 Hz, 1H), 2.38 (s, 1H), 2.21-2.05 (m, 1H), 1.91-1.76 (m, 1H), 1.62-1.47 (m, 2H), 1.35 (d, J=6.0 Hz, 3H), 0.96 (t, J=6.8 Hz, 3H), 0.70 (t, J=6.8 Hz, 3H). MS: M/e 501 (M+1)⁺.

Compound A103b (the later peak): ¹H NMR (400 MHz, CD₃OD) δ 7.93 (s, 1H), 7.68-7.55 (m, 4H), 5.55 (s, 1H), 5.47 (s, 2H), 4.75-3.85 (m, 1H), 3.75-3.64 (m, 1H), 3.51 (d, J=12.8 Hz, 1H), 3.43 (s, 3H), 3.30-3.29 (m, 1H), 3.16 (d, J=10.4 Hz, 1H), 2.67 (d, J=11.6 Hz, 1H), 2.30 (d, J=12.0 Hz, 1H), 2.05-1.85 (m, 1H), 1.80-1.45 (m, 3H), 1.33 (d, J=6.0 Hz, 3H), 1.03 (t, J=7.2 Hz, 3H), 0.62 (t, J=6.8 Hz, 3H). MS: M/e 501 (M+1)⁺.

### Compound A111: 2-(7-((2S,5R)-4-(1-(2-bromo-4-fluorophenyl)ethyl)-2,5-diethylpiperazin-1-yl)-4-methyl-5-oxo-4,5-dihydro-2H-pyrazolo[4,3-b]pyridin-2-yl)acetonitrile

### Step A: (2R,5S)-1-(1-(2-bromo-4-fluorophenyl)ethyl)-2,5-diethylpiperazine

Tert-butyl (2S,5R)-4-(1-(2-bromo-4-fluorophenyl)ethyl)-2,5-diethylpiperazine-1-carboxylate (150 mg) was dissolved in DCM (3 mL) was added TFA (2 ml). The mixture was stirred at RT for 4 hours. The mixture was concentrated to dryness and give the titled compound (200 mg, crude) used in the next step without purification. MS: M/e 343 (M+1)⁺.

### Step B: 7-((2S,5R)-4-(1-(2-bromo-4-fluorophenyl)ethyl)-2,5-diethylpiperazin-1-yl)-4-methyl-2-(tetrahydro-2H-pyran-2-yl)-2,4-dihydro-5H-pyrazolo[4,3-b]pyridin-5-one

To a solution of (2R,5S)-1-(1-(2-bromo-4-fluorophenyl)ethyl)-2,5-diethylpiperazine (34 mg, 0.1 mmol) in acetonitrile (2 mL) was added Intermediate 2 (76 mg, 0.2 mmol) and DIPEA (65 mg, 0.5 mmol). The mixture was stirred at 100° C. for weekend in a sealed tube. The reaction was quenched with water, extracted with EA, washed with brine, dried over Na₂SO₄, filtered, and concentrated to dryness. The resulting residue was purified by flash column chromatography (MeOH: DCM=0-10% in 20 minutes) to give the product (60 mg, crude). MS: M/e 574 (M+1)⁺.

### Step C: 7-((2S,5R)-4-(1-(2-bromo-4-fluorophenyl)ethyl)-2,5-diethylpiperazin-1-yl)-4-methyl-2,4-dihydro-5H-pyrazolo[4,3-b]pyridin-5-one

To a solution of 7-((2S,5R)-4-(1-(2-bromo-4-fluorophenyl)ethyl)-2,5-diethylpiperazin-1-yl)-4-methyl-2-(tetrahydro-2H-pyran-2-yl)-2,4-dihydro-5H-pyrazolo[4,3-b]pyridin-5-one (60 mg) in MeOH (1.5 mL) was added HCl(g) (4 M in dioxane, 2 ml). The reaction was stirred at room temperature for 4 hours. The solvent was removed under reduced pressure. The resulting residue (50 mg, crude) was used directly for next step without further purification. MS: M/e 490 (M+1)⁺.

### Step D: 2-(7-((2S,5R)-4-(1-(2-bromo-4-fluorophenyl)ethyl)-2,5-diethylpiperazin-1-yl)-4-methyl-5-oxo-4,5-dihydro-2H-pyrazolo[4,3-b]pyridin-2-yl)acetonitrile

To a solution of 7-((2S,5R)-4-(1-(2-bromo-4-fluorophenyl)ethyl)-2,5-diethylpiperazin-1-yl)-4-methyl-2,4-dihydro-5H-pyrazolo[4,3-b]pyridin-5-one (50 mg, crude) in DMF (2 mL) was added potassium carbonate (55 mg, 0.4 mmol) and 2-iodoacetonitrile (25 mg, 0.15 mmol). The resulting mixture was stirred at room temperature overnight. The reaction mixture was diluted with water, extracted with EA, washed with brine, dried over Na₂SO₄, filtered, and concentrated to dryness. The crude was purified by Prep-TLC (DCM:MeOH=13:1) to give titled compound (3 mg, 5%). ¹H NMR (400 MHz, CD3OD) δ 7.93 (d, J=2.4 Hz, 1H), 7.78-7.64 (m, 1H), 7.40-7.32 (m, 1H), 7.16 (dd, J=15.9, 7.3 Hz, 1H), 5.56 (s, 1H), 5.47 (d, J=3.5 Hz, 2H), 4.23-4.18 (m, 1H), 3.49 (d, J=14.5 Hz, 1H), 3.43 (s, 3H), 3.18-2.99 (m, 1H), 2.90-2.69 (m, 2H), 2.42-2.23 (m, 1H), 2.03-1.80 (m, 1H), 1.88-1.63 (m, 2H), 1.56 (d, J=7.9 Hz, 2H), 1.28-1.23 (m, 3H), 0.94-0.76 (m, 3H), 0.74-0.63 (m, 3H) ppm. MS: M/e 529 (M+1)⁺.

### Compound A115: 2-(7-((2S,5R)-2,5-diethyl-4-(1-(4-methyl-2-(trifluoromethyl)phenyl)ethyl)piperazin-1-yl)-4-methyl-5-oxo-4,5-dihydro-2H-pyrazolo[4,3-b]pyridin-2-yl)acetonitrile

### Step A: 2-(7-((2S,5R)-2,5-diethyl-4-(1-(4-methyl-2-(trifluoromethyl)phenyl)ethyl)piperazin-1-yl)-4-methyl-5-oxo-4,5-dihydro-2H-pyrazolo[4,3-b]pyridin-2-yl)acetonitrile

To a solution of Intermediate 5 (30 mg, 0.09 mmol) in CH₃CN (3 mL) and was added 1-(4-methyl-2-(trifluoromethyl)phenyl)ethan-1-ol (56 mg, 0.27 mmol), (cyanomethyl)trimethylphosphonium iodide (111 mg, 0.46 mmol) and DIPEA (118 mg, 0.9 mmol). The resulting mixture was stirred at 105° C. overnight. The reaction solvent was removed under reduce pressure to afford crude product Compound A115. The Compound A115 was purified by Prep-TLC (DCM:MeOH=20:1) and further separated into Compound A115a (1.23 mg) and Compound A115b (0.53 mg) by Prep-HPLC (Method A).

Compound A115a (the earlier peak): ¹H NMR (400 MHz, CD₃OD) δ 7.93 (s, 1H), 7.87 (d, J=8.0 Hz, 1H), 7.46-7.41 (m, 2H), 5.58-5.50 (m, 1H), 5.47 (s, 2H), 4.05-4.01 (m, 1H), 3.49-3.44 (m, 1H), 3.43 (s, 3H), 3.22-3.15 (m, 1H), 2.80-2.57 (m, 2H), 2.39 (s, 3H), 2.19 (d, J=11.0 Hz, 1H), 1.93-1.47 (m, 4H), 1.33-1.18 (m, 4H), 1.15-0.97 (m, 3H), 0.78-0.48 (m, 3H) ppm. MS: M/e 515 (M+1)⁺.

Compound A115b (the later peak): ¹H NMR (400 MHz, CD₃OD) δ 7.92 (s, 1H), 7.89 (d, J=8.4 Hz, 1H), 7.46 (s, 2H), 5.56 (s, 1H), 5.46 (s, 2H), 4.16 (s, 1H), 3.43 (s, 3H), 3.16-3.05 (m, 1H), 2.97-2.80 (m, 3H), 2.40 (s, 3H), 2.37-1.85 (m, 3H), 1.60-1.41 (m, 2H), 1.32-1.21 (m, 4H), 1.05-0.90 (m, 3H), 0.75-0.52 (m, 3H) ppm. MS: M/e 515 (M+1)⁺.

### Compound A119: 2-(7-((2S,5R)-2,5-diethyl-4-(1-(1-ethyl-5-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile

### Step A: N-methoxy-N-methyl-3-(trifluoromethyl)-1H-pyrazole-4-carboxamide

A mixture of 3-(trifluoromethyl)-1H-pyrazole-4-carboxylic acid (1 g, 5.56 mmol), N,O-dimethylhydroxylamine hydrochloride (0.65 g, 6.63 mmol), HATU (2.53 g, 6.66 mmol) and DIPEA (2.15 g, 16.67 mmol) in THF (15 ml) was stirred at RT overnight. After completed, the solution was concentrated under reduced pressure. The resulting residue was dissolved in EA (30 ml), washed with aq. NaHCO₃ (10 ml×2), brine (10 ml), dried over Na₂SO₄ and then concentrated to dryness. The resulting residue was purified by flash column chromatography (MeOH/DCM) to give the titled compound (1.2 g, 100%). MS: M/e 224 (M+1)⁺.

### Step B: 1-ethyl-N-methoxy-N-methyl-5-(trifluoromethyl)-1H-pyrazole-4-carboxamide

A mixture of N-methoxy-N-methyl-3-(trifluoromethyl)-1H-pyrazole-4-carboxamide (1.2 g, 5.38 mmol), iodoethane (0.97 g, 6.22 mmol) and K₂CO₃ (2.23 g, 16.16 mmol) in MeCN (15 ml) was stirred at 80° C. overnight. After completed, the solution was concentrated under reduced pressure. The resulting residue was purified by flash column chromatography (PE/EA) to give the titled compound (140 mg). MS: M/e 252 (M+1)⁺.

### Step C: 1-(1-ethyl-5-(trifluoromethyl)-1H-pyrazol-4-yl)ethan-1-one

To a solution of 1-ethyl-N-methoxy-N-methyl-5-(trifluoromethyl)-1H-pyrazole-4-carboxamide (140 mg, 0.56 mmol) in THF (6 ml) at −60° C. under N₂, was added MeMgBr (3M, 0.37 ml, 1.11 mmol) dropwise. The solution was warmed to RT naturally and stirred at RT overnight. After completed, the solution was quenched with water (2 ml) and concentrated under reduced pressure. The resulting residue was purified by Prep-TLC with PE:EA (2:1) to give the titled compound (55 mg, 48%). MS: M/e 207 (M+1)⁺.

### Step D: 1-(1-ethyl-5-(trifluoromethyl)-1H-pyrazol-4-yl)ethan-1-ol

A mixture of 1-(1-ethyl-5-(trifluoromethyl)-1H-pyrazol-4-yl)ethan-1-one (55 mg, 0.27 mmol) and NaBH₄ (11 mg, 0.29 mmol) in MeOH (6 ml) was stirred at RT overnight. After completed, the solution was concentrated under reduced pressure. The resulting residue was dissolved in EA (10 ml), washed with aq. NH₄Cl (6 ml) and brine (6 ml), dried and then concentrated to afford crude product (55 mg, 100%), which was used directly for the next step without further purification. MS: M/e 209 (M+1)⁺.

### Step E: 2-(7-((2S,5R)-2,5-diethyl-4-(1-(1-ethyl-5-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile

A solution of Intermediate 5 (43 mg, 0.13 mmol), 1-(1-ethyl-5-(trifluoromethyl)-1H-pyrazol-4-yl)ethan-1-ol (27 mg, 0.13 mmol), (cyanomethyl)trimethylphosphonium iodide (95 mg, 0.39 mmol) and DIPEA (84 mg, 0.65 mmol) in MeCN (2 ml) was stirred at 100° C. overnight. After completed, the solution was concentrated under reduced pressure, obtain Compound A119. The resulting residue containing Compound A119 was purified Prep-HPLC to give the titled Compound A119a (0.65 mg) and Compound A119b (0.75 mg).

Compound A119a (the earlier peak): ¹H NMR (400 MHz, DMSO-d6) δ 7.98 (s, 1H), 7.71 (s, 1H), 5.62 (s, 2H), 5.37 (s, 1H), 4.25 (q, J=7.1 Hz, 2H), 3.98 (q, J=6.7 Hz, 1H), 3.32-3.31 (m, 1H), 3.27 (s, 3H), 3.16 (d, J=10.8 Hz, 1H), 2.98 (s, 1H), 2.81 (dd, J=11.9, 3.9 Hz, 1H), 2.75-2.66 (m, 1H), 2.40 (d, J=10.8 Hz, 1H), 1.91-1.78 (m, 1H), 1.51-1.47 (m, 2H), 1.36 (t, J=7.2 Hz, 4H), 1.31 (d, J=6.5 Hz, 3H), 0.79 (s, 3H), 0.71 (t, J=7.3 Hz, 3H) ppm. MS: M/e 519 (M+1)⁺.

Compound A119b (the later peak): ¹H NMR (400 MHz, DMSO-d6) δ 7.99 (s, 1H), 7.66 (s, 1H), 5.62 (s, 2H), 5.39 (s, 1H), 4.25 (q, J=7.2 Hz, 2H), 3.78 (q, J=6.5 Hz, 1H), 3.28 (s, 2H), 3.27 (s, 3H), 2.98 (d, J=10.2 Hz, 2H), 2.55 (d, J=9.5 Hz, 1H), 2.35 (d, J=11.9 Hz, 1H), 1.89-1.78 (m, 1H), 1.60-1.40 (m, 2H), 1.35 (t, J=7.2 Hz, 4H), 1.27 (d, J=6.5 Hz, 3H), 0.91 (t, J=7.3 Hz, 3H), 0.61 (t, J=7.1 Hz, 3H) ppm. MS: M/e 519 (M+1)⁺.

### Compound A126: 2-(7-((2S,5R)-4-(1-(2,3-dihydrobenzo[b][1,4]dioxin-6-yl)ethyl)-2,5-diethylpiperazin-1-yl)-4-methyl-5-oxo-4,5-dihydro-2H-pyrazolo[4,3-b]pyridin-2-yl)acetonitrile

### Step A: tert-butyl (2S,5R)-4-(1-(2,3-dihydrobenzo[b][1,4]dioxin-6-yl)ethyl)-2,5-diethylpiperazine-1-carboxylate

A mixture of 1-(2,3-dihydrobenzo[b][1,4]dioxin-6-yl)ethan-1-ol (670 mg, 3.7 mmol), tert-butyl (2S,5R)-2,5-diethylpiperazine-1-carboxylate (600 mg, 2.48 mmol), (cyanomethyl)trimethylphosphonium iodide (1.5 g, 6.2 mmol) and DIPEA (1.6 g, 12.4 mmol) in MeCN (10 mL) was stirred at 100° C. for 16 hours. The mixture was diluted with EA (20 mL), washed with brine (20 mL×3), dried over Na₂SO₄ and concentrated to dryness. The resulting residue was purified by flash column chromatography to give the titled compound (410 mg, 41%). MS: M/e 405 (M+1)⁺.

### Step B: (2R,5S)-1-(1-(2,3-dihydrobenzo[b][1,4]dioxin-6-yl)ethyl)-2,5-diethylpiperazine

To a stirred solution of tert-butyl (2S,5R)-4-(1-(2,3-dihydrobenzo[b][1,4]dioxin-6-yl)ethyl)-2,5-diethylpiperazine-1-carboxylate (360 mg, 0.89 mmol) in EA (5 mL) was added HCl (5 mL, 4 M in 1,4-dioxane). The resulting mixture was stirred at room temperature for 16 hours. The mixture was concentrated to dryness to give the titled compound (380 mg, HCl salt). MS: M/e 305 (M+1)⁺.

### Step C: 7-((2S,5R)-4-(1-(2,3-dihydrobenzo[b][1,4]dioxin-6-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one

A mixture of (2R,5S)-1-(1-(2,3-dihydrobenzo[b][1,4]dioxin-6-yl)ethyl)-2,5-diethylpiperazine (160 mg, 0.52 mmol), Intermediate 2 (200 mg, 0.52 mmol) and DIPEA (500 mg, 3.87 mmol) in MeCN (3 mL) was stirred at 100° C. for 16 hours. The mixture was diluted with EA (10 mL), washed with brine (10 mL×3), dried over NaSO₄ and concentrated to dryness. The resulting residue was the resulting residue was purified by flash column chromatography to give the titled compound (70 mg, 25%). MS: M/e 536 (M+1)⁺.

### Step D: 7-((2S,5R)-4-(1-(2,3-dihydrobenzo[b][1,4]dioxin-6-yl)ethyl)-2,5-diethylpiperazin-1-yl)-4-methyl-2,4-dihydro-5H-pyrazolo[4,3-b]pyridin-5-one

To a stirred solution of 7-((2S,5R)-4-(1-(2,3-dihydrobenzo[b][1,4]dioxin-6-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one (65 mg, 0.12 mmol) in MeOH (3 mL) was added HCl (2 mL, 4 M in 1,4-dioxane). The resulting mixture was stirred at room temperature for 20 hours. The mixture was concentrated to dryness to give the titled compound as a HCl salt (55 mg, 94%). MS: M/e 452 (M+1)⁺.

### Step E: 2-(7-((2S,5R)-4-(1-(2,3-dihydrobenzo[b][1,4]dioxin-6-yl)ethyl)-2,5-diethylpiperazin-1-yl)-4-methyl-5-oxo-4,5-dihydro-2H-pyrazolo[4,3-b]pyridin-2-yl)acetonitrile

To a solution of 7-((2S,5R)-4-(1-(2,3-dihydrobenzo[b][1,4]dioxin-6-yl)ethyl)-2,5-diethylpiperazin-1-yl)-4-methyl-2,4-dihydro-5H-pyrazolo[4,3-b]pyridin-5-one (55 mg, 0.11 mmol) and a solution of K₂CO₃ aq. (3M, 0.5 mL) in MeCN (3 mL) was added 2-iodoacetonitrile (38 mg, 0.22 mmol). The resulting mixture was stirred at room temperature for 20 hours. The mixture was diluted with EA (10 mL), washed with brine (5 mL×3), dried over Na₂SO₄ and concentrated to dryness. The resulting residue was purified by Prep-HPLC (Method A) to give the titled compound (12 mg, 22%). ¹H NMR (400 MHz, CD₃OD) δ 7.92 (s, 1H), 6.90-6.70 (m, 3H), 5.54 (s, 1H), 5.47 (s, 2H), 4.22 (d, J=6.8 Hz, 4H), 3.68-3.55 (m, 1H), 3.52-3.38 (m, 4H), 3.30-3.21 (m, 2H), 3.15-2.93 (m, 1H), 2.89-2.54 (m, 1H), 2.53-2.39 (m, 1H), 2.17-1.87 (m, 1H), 1.85-1.44 (m, 3H), 1.34-1.21 (m, 3H), 1.05-0.91 (m, 3H), 0.75-0.61 (m, 3H) ppm. MS: M/e 491 (M+1)⁺.

### Compound A127: 2-(7-((2S,5R)-2,5-diethyl-4-(1-(p-tolyl)ethyl)piperazin-1-yl)-4-methyl-5-oxo-4,5-dihydro-2H-pyrazolo[4,3-b]pyridin-2-yl)acetonitrile

### Step A: tert-butyl (2S,5R)-2,5-diethyl-4-(1-(p-tolyl)ethyl)piperazine-1-carboxylate

A mixture of 1-(p-tolyl)ethan-1-ol (1 g, 7.35 mmol), tert-butyl (2S,5R)-2,5-diethylpiperazine-1-carboxylate (1.8 g, 7.44 mmol), (cyanomethyl)trimethylphosphonium iodide (3.6 g, 14.81 mmol) and DIPEA (5.7 g, 44.19 mmol) in MeCN (10 ml) was stirred at 100° C. overnight. After completed, the solution was concentrated under reduced pressure. The resulting residue was purified by flash column flash column chromatography with 0-5% EA in PE to give the titled compound (1.66 g, 63%). MS: M/e 361 (M+1)⁺.

### Step B: (2R,5S)-2,5-diethyl-1-(1-(p-tolyl)ethyl)piperazine

A mixture of tert-butyl (2S,5R)-2,5-diethyl-4-(1-(p-tolyl)ethyl)piperazine-1-carboxylate (1.66 g, 4.61 mmol) and TFA (4 ml) in DCM (20 ml) was stirred at RT for 2 hours. After completed, the solution was concentrated under reduced pressure. The resulting residue was dissolved in EA (20 ml), washed with aq. NaHCO₃ (15 ml), brine (15 ml), dried and concentrated under reduced pressure to give the titled compound (1.19 g, 100%), which was used directly for the next step without further purification. MS: M/e 261 (M+1)⁺.

### Step C: 7-((2S,5R)-2,5-diethyl-4-(1-(p-tolyl)ethyl)piperazin-1-yl)-4-methyl-2-(tetrahydro-2H-pyran-2-yl)-2,4-dihydro-5H-pyrazolo[4,3-b]pyridin-5-one

A mixture of (2R,5S)-2,5-diethyl-1-(1-(p-tolyl)ethyl)piperazine (1.19 g, 4.58 mmol), Intermediate 2 (1.5 g, 3.94 mmol) and DIPEA (1.52 g, 11.78 mmol) in MeCN (10 ml) was stirred at 105° C. overnight. After completed, the solution was concentrated under reduced pressure and then purified by flash column chromatography (MeOH/DCM) to give the titled compound (1.6 g, 83%). MS: M/e 492 (M+1)⁺.

### Step D: 7-((2S,5R)-2,5-diethyl-4-(1-(p-tolyl)ethyl)piperazin-1-yl)-4-methyl-2,4-dihydro-5H-pyrazolo[4,3-b]pyridin-5-one

A mixture of 7-((2S,5R)-2,5-diethyl-4-(1-(p-tolyl)ethyl)piperazin-1-yl)-4-methyl-2-(tetrahydro-2H-pyran-2-yl)-2,4-dihydro-5H-pyrazolo[4,3-b]pyridin-5-one (1.6 g, 3.26 mmol) and HCl/dioxane (4M, 4 ml) in MeOH (16 ml) was stirred at RT for overnight. After completed, the solution was concentrated under reduced pressure to give the titled compound (1.3 g, 100%), which was used directly for the next step without further purification. MS: M/e 408 (M+1)⁺.

### Step E: 2-(7-((2S,5R)-2,5-diethyl-4-(1-(p-tolyl)ethyl)piperazin-1-yl)-4-methyl-5-oxo-4,5-dihydro-2H-pyrazolo[4,3-b]pyridin-2-yl)acetonitrile

A mixture of 7-((2S,5R)-2,5-diethyl-4-(1-(p-tolyl)ethyl)piperazin-1-yl)-4-methyl-2,4-dihydro-5H-pyrazolo[4,3-b]pyridin-5-one (1.3 g, 3.19 mmol), 2-iodoacetonitrile (0.67 g, 4.01 mmol) and K₂CO₃ (0.88 g, 6.38 mmol) in DMF (15 ml) was stirred at RT overnight and then 60° C. for 24 hours. After completed, the mixture was poured into water (50 ml) and then extracted with EA (20 ml×2). The organic layer was washed with brine (15 ml), dried and concentrated under reduced pressure. The resulting residue was purified by flash column chromatography (MeOH/DCM) and the fractions was concentrated under reduced pressure to give the titled compound Compound A127, which was further separated into Compound A127a (118 mg) and Compound A127b (76 mg) by Prep-HPLC (Method B).

Compound A127: ¹H NMR (400 MHz, CD₃OD) δ 7.92 (d, J=2.4 Hz, 1H), 7.24 (dd, J=11.4, 8.0 Hz, 2H), 7.14 (dd, J=18.0, 7.7 Hz, 3H), 5.54 (s, 1H), 5.46 (d, J=3.5 Hz, 2H), 3.73-3.47 (m, 1H), 3.43 (s, 3H), 3.20-2.97 (m, 1H), 2.73 (m, 1H), 2.40 (d, J=11.6 Hz, 1H), 2.32 (d, J=5.6 Hz, 3H), 2.15-1.77 (m, 2H), 1.71-1.46 (m, 3H), 1.31 (dd, J=15.6, 6.4 Hz, 3H), 0.96-0.86 (m, 4H), 0.67-0.60 (m, 3H) ppm. MS: M/e 447 (M+1)⁺.

Compound A127a (the earlier peak): ¹H NMR (400 MHz, DMSO-d6) δ ppm 7.98 (s, 1H), 7.22 (d, J=7.9 Hz, 2H), 7.14 (d, J=7.9 Hz, 2H), 5.61 (s, 2H), 5.39 (s, 1H), 3.64 (q, J=6.4 Hz, 1H), 3.34 (s, 2H), 3.27 (s, 3H), 3.07 (d, J=11.7 Hz, 1H), 2.90 (d, J=11.6 Hz, 1H), 2.73 (d, J=8.5 Hz, 1H), 2.34-2.27 (m, 4H), 2.07-1.93 (m, 1H), 1.66-1.54 (m, 1H), 1.54-1.36 (m, 2H), 1.25 (d, J=6.3 Hz, 3H), 0.86 (t, J=7.3 Hz, 3H), 0.59 (t, J=7.3 Hz, 3H) ppm. MS: M/e 447 (M+1)⁺.

Compound A127b (the later peak): ¹H NMR (400 MHz, DMSO-d6) δ ppm 7.98 (s, 1H), 7.24 (d, J=7.9 Hz, 2H), 7.12 (d, J=7.8 Hz, 2H), 5.61 (s, 2H), 5.38 (s, 1H), 3.52 (q, J=6.4 Hz, 1H), 3.36 (s, 1H), 3.34 (s, 2H), 3.27 (s, 3H), 3.02 (d, J=9.2 Hz, 1H), 2.47 (s, 1H), 2.30-2.23 (m, 4H), 1.93-1.78 (m, 1H), 1.61-1.39 (m, 3H), 1.21 (d, J=6.4 Hz, 3H), 0.94 (t, J=7.2 Hz, 3H), 0.54 (t, J=6.9 Hz, 3H) ppm. MS: M/e 447 (M+1)⁺.

### Compound A129: 2-(7-((2S,5R)-2,5-diethyl-4-((S)-1-(quinoxalin-6-yl)ethyl)piperazin-1-yl)-5-oxo-4,5-dihydro-2H-pyrazolo[4,3-b]pyridin-2-yl)acetonitrile

### Step A: 7-((2S,5R)-2,5-diethyl-4-(1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-b]pyridin-5-one

To a solution of Intermediate 7 (500 mg, 1 mmol) and 6-(1-((2R,5S)-2,5-diethylpiperazin-1-yl)ethyl)quinoxaline (360 mg, 1.2 mmol) in CH₃CN (8 mL) was added DIPEA (258 mg, 2 mmol). Then the mixture was heated at 90° C. under N₂ for 56 hours. The mixture was cooled to room temperature, diluted with water (50 mL), extracted with EA (80 mL×2). The combined organic layers were washed with brine, dried over Na₂SO₄, filtered and concentrated to dryness. The resulting residue was purified by flash column chromatography to give titled compound (410 mg, 64%). MS: M/e 636 (M+1)⁺.

### Step B: 7-((2S,5R)-2,5-diethyl-4-(1-(quinoxalin-6-yl)ethyl)piperazin-1-yl)-2,4-dihydro-5H-pyrazolo[4,3-b]pyridin-5-one

To a solution of 7-((2S,5R)-2,5-diethyl-4-(1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-b]pyridin-5-one (410 mg, 0.64 mmol) in TFA (4 mL) was added trifluoromethanesulfonic acid (6 mL) was add. The resulting mixture was stirred at room temperature overnight. The mixture was quenched with ice-water, basified with saturated Na₂CO₃ solution to pH 7-8, extracted with DCM:IPA (6:1, 60 mL×3). The combined organic layers were washed with brine, dried over Na₂SO₄, filtered and concentrated. The resulting residue was purified by Prep-TLC to give titled compound (71 mg, 25%). MS: M/e 432 (M+1)⁺.

### Step C: 2-(7-((2S,5R)-2,5-diethyl-4-((S)-1-(quinoxalin-6-yl)ethyl)piperazin-1-yl)-5-oxo-4,5-dihydro-2H-pyrazolo[4,3-b]pyridin-2-yl)acetonitrile

To a solution of 7-((2S,5R)-2,5-diethyl-4-(1-(quinoxalin-6-yl)ethyl)piperazin-1-yl)-2,4-dihydro-5H-pyrazolo[4,3-b]pyridin-5-one (71 mg, 0.16 mmol) and K₂CO₃ (47 mg, 0.34 mmol) in DMF (2 mL) was added 2-iodoacetonitrile (42 mg, 0.25 mmol). The reaction was stirred at room temperature for 56 hours. The reaction mixture was diluted with water, extracted with EA (60 mL), washed with brine, dried over Na₂SO₄, filtered, and concentrated to dryness. The resulting residue was purified by flash column chromatography to give titled compound (14 mg, 18%). ¹HNMR (400 MHz, CD₃OD) δ 8.94-8.84 (m, 2H), 8.16-7.95 (m, 3H), 7.77-7.72 (m, 1H), 5.52-5.40 (m, 3H), 4.14-3.85 (m, 1H), 3.64-3.53 (m, 0.5H), 3.40-3.20 (m, 2.5H), 3.27-3.18 (m, 0.5H), 3.14-3.04 (m, 0.5H), 2.99-2.90 (m, 0.5H), 2.80-2.70 (m, 0.5H), 2.48-2.32 (m, 1H), 2.25-2.10 (m, 0.5H), 2.05-1.83 (m, 1H), 1.80-1.54 (m, 2.5H), 1.50-1.38 (m, 3H), 1.07 (t, J=7.6 Hz, 1.5H), 0.99 (t, J=7.6 Hz, 1.5H), 0.69 (t, J=7.6 Hz, 1.5H), 0.57 (t, J=7.6 Hz, 1.5H) ppm. MS: M/e 471 (M+1)⁺.

### Compound A133: 2-(6-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 2-(7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile (30 mg, 0.07 mmol) (Compound A2a) in CH₃CN (5 mL) were added NCS (10 mg, 0.08 mmol) at room temperature. The resulting mixture was stirred at room temperature for another 2 hours. The reaction mixture was diluted with EA (20 mL) and washed with water (10 mL×2). The organic layers were concentrated and purified by pre-TLC (DCM:MeOH=15:1) to give the titled compound (12 mg, 38%) as a single diastereoisomer. ¹H NMR (400 MHz, CD₃OD) δ 8.89-8.87 (m, 2H), 8.14-8.03 (m, 3H), 7.98 (s, 1H), 5.50 (s, 2H), 4.62-4.58 (m, 1H), 4.20-4.16 (m, 1H), 3.87-3.80 (m, 1H), 3.53 (s, 3H), 3.32-3.31 (m, 1H), 3.15-3.10 (m, 1H), 2.74-2.59 (m, 2H), 1.51 (d, J=6.7 Hz, 3H), 1.29 (d, J=6.4 Hz, 3H), 1.07 (d, J=6.4 Hz, 3H) ppm. MS: M/e 491 (M+1)⁺.

### Compound A134: 2-(cyanomethyl)-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridine-6-carbonitrile

### Step A: 2-(6-bromo-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 2-(7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile (80 mg, 0.18 mmol) (Compound A2a) in CH₃CN (5 mL) were added NBS (35 mg, 0.20 mmol) slowly at room temperature. The resulting mixture was stirred at room temperature for another 2 hours. The reaction mixture was diluted with EA (20 mL) and washed with water (10 mL×2). The organic layers were concentrated and purified by pre-TLC (DCM:MeOH=15:1) to give the titled compound (40 mg, 43%). MS: M/e 535 (M+1)⁺.

### Step B: 2-(cyanomethyl)-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridine-6-carbonitrile

A mixture of 2-(6-bromo-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile (40 mg, 0.08 mmol), Pd(PPh₃)₄ (23 mg, 0.02 mmol) and Zn(CN)₂ (19 mg, 0.16 mmol) in DMF (2 mL) was heated to 100° C. for overnight under N₂ atmosphere. The reaction mixture was diluted with EA (10 mL) and washed with brine (5 mL×2). The organic layers were concentrated and purified by pre-TLC (DCM:MeOH=15:1) to give the titled compound (13.8 mg, 38%). ¹H NMR (400 MHz, CD₃OD) δ 8.88 (d, J=3.4 Hz, 2H), 8.14-8.05 (m, 3H), 7.99 (s, 1H), 5.50 (s, 2H), 4.01-3.96 (m, 2H), 3.43 (s, 3H), 3.18-3.15 (m, 1H), 3.02-2.97 (m, 2H), 2.81-2.72 (m, 1H), 1.66 (d, J=6.7 Hz, 3H), 1.44 (d, J=6.4 Hz, 3H), 1.01 (d, J=6.6 Hz, 3H) ppm. MS: M/e 482 (M+1)⁺.

### Compound A136: 2-(7-((2S,5R)-2,5-diethyl-4-(1-(5-fluoro-3-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile

### Step A: 3-bromo-5-fluoro-N-methoxy-N-methylpicolinamide

A mixture of 3-bromo-5-fluoropicolinic acid (1 g, 4.57 mmol), N,O-dimethylhydroxylamine hydrochloride (0.54 g, 5.51 mmol), HATU (2.1 g, 5.53 mmol) and DIPEA (1.8 g, 13.95 mmol) in THF (10 ml) was stirred at RT for 5 hours. After completed, the solution was diluted with EA (20 ml), washed with aq. NaHCO₃ (10 ml), brine (10 ml), dried over anhydrous Na₂SO₄ and then concentrated under reduced pressure. The resulting residue was purified by flash column chromatography (EA/PE) to give the titled compound (1.1 g, 92%). ¹H NMR (400 MHz, CDCl₃) δ 8.45 (d, J=2.1 Hz, 1H), 7.71 (dd, J=7.6, 2.2 Hz, 1H), 3.57 (s, 3H), 3.41 (s, 3H) ppm.

### Step B: 5-fluoro-N-methoxy-N-methyl-3-(trifluoromethyl)picolinamide

A mixture of 3-bromo-5-fluoro-N-methoxy-N-methylpicolinamide (1 g, 3.80 mmol), methyl 2,2-difluoro-2-(fluorosulfonyl)acetate (0.8 g, 4.17 mmol) and CuI (220 mg, 1.16 mmol) in DMF (10 ml) was stirred at 120° C. under N₂ overnight. After completed, the mixture was poured into water (20 ml), extracted with EA (15 ml×2). The organic layer was washed with brine (10 ml), dried and concentrated under reduced pressure. The resulting residue was purified by flash column chromatography (PE/EA) to give the titled compound (0.84 g, 88%). MS: M/e 253 (M+1)⁺.

### Step C: 1-(5-fluoro-3-(trifluoromethyl)pyridin-2-yl)ethan-1-one

To a solution of 5-fluoro-N-methoxy-N-methyl-3-(trifluoromethyl)picolinamide (0.84 g, 3.33 mmol) in THF (10 ml) at −60° C. under N₂, was added MeMgBr (3M, 1.22 ml, 3.66 mmol) dropwise. The solution was warmed to RT naturally and stirred at RT overnight. After completed, the solution was quenched with water (10 ml), extracted with EA (15 ml×2). The organic layer was washed with brine (10 ml), dried and concentrated under reduced pressure. The resulting residue was purified by flash column chromatography (PE/EA) to give the titled compound (245 mg, 36%). MS: M/e 208 (M+1)⁺. ¹H NMR (400 MHz, CDCl₃) δ 8.64 (d, J=2.5 Hz, 1H), 7.82 (dd, J=8.4, 2.6 Hz, 1H), 2.69 (s, 3H) ppm.

### Step D: 1-(5-fluoro-3-(trifluoromethyl)pyridin-2-yl)ethan-1-ol

A mixture of 1-(5-fluoro-3-(trifluoromethyl)pyridin-2-yl)ethan-1-one (245 mg, 1.18 mmol) and NaBH₄ (45 mg, 1.18 mmol) in MeOH (4 ml) was stirred at RT for 1.5 hours. After completed, the solution was quenched with water (1 ml) and then concentrated under reduced pressure. The resulting residue was dissolved in EA (15 ml), washed with aq. NaHCO₃ (10 ml), brine (10 ml), dried and concentrated to give the titled compound (210 mg, 85%), which was used directly for the next step without further purification. ¹H NMR (400 MHz, CDCl₃) δ 8.64 (d, J=2.4 Hz, 1H), 7.70 (dd, J=8.2, 2.6 Hz, 1H), 5.18 (q, J=6.5 Hz, 1H), 1.47 (d, J=6.4 Hz, 3H) ppm.

### Step E: 2-(7-((2S,5R)-2,5-diethyl-4-(1-(5-fluoro-3-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile

A mixture of Intermediate 5 (50 mg, 0.15 mmol), 1-(5-fluoro-3-(trifluoromethyl)pyridin-2-yl)ethan-1-ol (48 mg, 0.23 mmol), (cyanomethyl)trimethylphosphonium iodide (111 mg, 0.46 mmol) and DIPEA (197 mg, 1.53 mmol) in MeCN (2 ml) was stirred at 100° C. for 3 days. After completed, the solution was diluted with EA (15 ml), washed with brine (10 ml), dried and concentrated under reduced pressure. The resulting residue was purified by Prep-HPLC to give the titled compound (2.86 mg). ¹H NMR (400 MHz, DMSO-d6) δ 8.93 (d, J=6.3 Hz, 1H), 8.34 (s, 0.48H, HCOOH), 8.19 (td, J=9.2, 6.8 Hz, 1H), 7.97 (d, J=2.0 Hz, 1H), 5.61 (s, 2H), 5.36 (d, J=10.4 Hz, 1H), 4.30 (s, 1H), 4.15 (s, 1H), 3.26 (s, 4H), 3.21-3.09 (m, 1H), 3.03 (s, 2H), 2.80 (d, J=10.4 Hz, 0.5H), 2.24 (d, J=11.0 Hz, 0.5H), 1.88-1.72 (m, 0.5H), 1.58 (s, 1.5H), 1.37 (dd, J=31.7, 6.4 Hz, 4.5H), 1.11 (s, 0.5H), 0.86 (t, J=7.2 Hz, 1.5H), 0.77 (t, J=7.2 Hz, 1.5H), 0.69 (t, J=7.8 Hz, 1.5H), 0.54 (t, J=7.8 Hz, 1.5H) ppm. MS: M/e 520 (M+1)⁺.

### Compound A137: 2-(7-((2S,5R)-5-ethyl-4-(1-(4-fluoro-2-methoxyphenyl)ethyl)-2-methylpiperazin-1-yl)-4-methyl-5-oxo-4,5-dihydro-2H-pyrazolo[4,3-b]pyridin-2-yl)acetonitrile

### Step A: 1-(4-fluoro-2-methoxyphenyl)ethan-1-ol

To a solution of 1-(4-fluoro-2-methoxyphenyl)ethan-1-one (2.0 g, 11.89 mmol) in MeOH (30 mL) at 0° C. was added NaBH₄ (497 mg, 13.08 mmol). The mixture was stirred at 0° C. for 0.5 hours. Then the mixture was quenched with saturated NH₄Cl (20 mL) and extracted with EA (45 mL×3). The combined organic layers were washed with brine (20 mL×3), dried over Na₂SO₄ and concentrated under reduced pressure. The resulting residue was purified by flash column chromatography to give the titled compound (1.3 mg, 64%). MS: M/e 171 (M+1)⁺.

### Step B: tert-butyl (2S,5R)-5-ethyl-4-(1-(4-fluoro-2-methoxyphenyl)ethyl)-2-methylpiperazine-1-carboxylate

To a solution of 1-(4-fluoro-2-methoxyphenyl)ethan-1-ol (335 mg, 1.97 mmol), tert-butyl (2S,5R)-5-ethyl-2-methylpiperazine-1-carboxylate (450 mg, 1.97 mmol) and (cyanomethyl)trimethylphosphonium iodide (955 mg, 3.93 mmol) in CH₃CN (2 mL) was added DIPEA (1270 mg, 9.85 mmol). The mixture solution was degassed 3 times under N₂ atmosphere. Then the mixture solution was stirred at 105° C. for 24 hours. The reaction mixture was quenched with saturated NH₄Cl (20 mL) at room temperature and extracted with EA (35 mL×2). The combined organic layers were dried over Na₂SO₄ and concentrated under reduced pressure. The resulting residue was purified by flash column chromatography to give the titled compound (180 mg, 24%). MS: M/e 381 (M+1)⁺.

### Step C: (2R,5S)-2-ethyl-1-(1-(4-fluoro-2-methoxyphenyl)ethyl)-5-methylpiperazine

To a solution of tert-butyl (2S,5R)-5-ethyl-4-(1-(4-fluoro-2-methoxyphenyl)ethyl)-2-methylpiperazine-1-carboxylate (90 mg, 0.237 mmol) in DCM (10 mL) at room temperature was added HCl (4 mL, 4 M in 1,4-dioxane). The mixture was stirred at room temperature for 2 hours. Then the mixture was concentrated under reduced pressure to give the crude product (HCl salt). The crude product was basified by Na₂CO₃ (4M) to pH ˜10 and extracted with EA (30 mL×3). The combined organic layers were washed with brine (20 mL×3), dried over Na₂SO₄ and concentrated under reduced pressure to give the titled compound (50 mg, 76%), which was used to next step directly without purification. MS: M/e 281 (M+1)⁺.

### Step D: 7-((2S,5R)-5-ethyl-4-(1-(4-fluoro-2-methoxyphenyl)ethyl)-2-methylpiperazin-1-yl)-4-methyl-2-(tetrahydro-2H-pyran-2-yl)-2,4-dihydro-5H-pyrazolo[4,3-b]pyridin-5-one

To a solution of (2R,5S)-2-ethyl-1-(1-(4-fluoro-2-methoxyphenyl)ethyl)-5-methylpiperazine (45 mg, 0.160 mmol) and Intermediate 2 (73 mg, 0.192 mmol) in CH₃CN (1 mL) at room temperature was added DIPEA (103 mg, 0.80 mmol). The mixture was stirred at 105° C. for 24 hours. Then the mixture was concentrated under reduced pressure. The resulting residue was purified by flash column chromatography to give the titled compound (50 mg, 61%). MS: M/e 512 (M+1)⁺.

### Step E: 7-((2S,5R)-5-ethyl-4-(1-(4-fluoro-2-methoxyphenyl)ethyl)-2-methylpiperazin-1-yl)-4-methyl-2,4-dihydro-5H-pyrazolo[4,3-b]pyridin-5-one

To a solution of 7-((2S,5R)-5-ethyl-4-(1-(4-fluoro-2-methoxyphenyl)ethyl)-2-methylpiperazin-1-yl)-4-methyl-2-(tetrahydro-2H-pyran-2-yl)-2,4-dihydro-5H-pyrazolo[4,3-b]pyridin-5-one (50 mg, 0.1 mmol) in MeOH (4 mL) at room temperature was added HCl (2 mL, 4 M in 1,4-dioxane). The mixture was stirred at room temperature for 2 hours. Then the mixture was concentrated under reduced pressure to give the crude product (HCl salt). The crude product was dissolved into water and basified by Na₂CO₃ (4M) to pH ˜10. The resulting mixture was extracted with EA (30 mL×2). The combined organic layers were washed with brine (20 mL×3), dried over Na₂SO₄ and concentrated under reduced pressure to give the titled compound (35 mg, 81%). MS: M/e 428 (M+1)⁺.

### Step F: 2-(7-((2S,5R)-5-ethyl-4-(1-(4-fluoro-2-methoxyphenyl)ethyl)-2-methylpiperazin-1-yl)-4-methyl-5-oxo-4,5-dihydro-2H-pyrazolo[4,3-b]pyridin-2-yl)acetonitrile

To a solution of 7-((2S,5R)-5-ethyl-4-(1-(4-fluoro-2-methoxyphenyl)ethyl)-2-methylpiperazin-1-yl)-4-methyl-2,4-dihydro-5H-pyrazolo[4,3-b]pyridin-5-one (35 mg, 0.073 mmol) and K₂CO₃ (20 mg, 0.146 mmol) in DMF (5 mL) at room temperature was added 2-iodoacetonitrile (24 mg, 0.146 mmol). The mixture solution was stirred at room temperature for 12 hours. Then the reaction mixture was quenched with saturated NaCl (20 mL) at room temperature and extracted with EA (30 mL×2). The combined organic layers were dried over Na₂SO₄ and concentrated under reduced pressure. The resulting residue was purified by Prep-HPLC (Method A) to give Compound A137 (9 mg). ¹H NMR (400 MHz, CD₃OD) δ 7.92 (s, 1H), 7.50-7.47 (m, 1H), 6.78-6.69 (m, 2H), 5.56 (s, 1H), 5.47 (s, 2H), 4.61-4.35 (m, 3H), 3.84 (s, 3H), 3.34 (s, 3H), 3.28-3.26 (m, 1H), 3.05-2.95 (m, 1H), 2.78-2.75 (m, 1H), 2.51-2.46 (m, 1H), 1.36-1.34 (m, 2H), 1.30-1.24 (m, 6H), 0.80-0.76 (m, 3H) ppm. MS: M/e 467 (M+1)⁺.

### Compound A143: 2-(7-((2R,5R)-2-(methoxymethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: tert-butyl (2R,5R)-5-(methoxymethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate

To a stirred solution of tert-butyl (2R,5R)-5-(hydroxymethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (92.2 mg, 0.2 mmol) in THF (5 mL) was added NaH (60%, 24 mg, 0.6 mmol) at 0° C. After the addition, the reaction mixture was stirred for 2 days. The reaction mixture was quenched with saturated NH₄Cl aq., extracted with EA (10 mL×2). The combined organic layers were washed with brine, dried over Na₂SO₄ and concentrated to dryness. The resulting residue was purified by Prep-TLC (CH₂Cl₂/MeOH=10/1) to give the titled compound (76 mg, 80%). MS: M/e 476 (M+1)⁺.

### Step B: 7-((2R,5R)-2-(methoxymethyl)-5-methylpiperazin-1-yl)-4-methyl-2-(tetrahydro-2H-pyran-2-yl)-2,4-dihydro-5H-pyrazolo[4,3-b]pyridin-5-one

To a stirred solution of tert-butyl (2R,5R)-5-(methoxymethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (76 mg, 0.165 mmol) in CH₂Cl₂ (5 mL) was added TFA (1 mL). After the addition, the reaction mixture was stirred for 4 hours. The reaction mixture was basified with saturated Na₂CO₃ aq. and extracted with EA (10 mL×3). The combined organic layers were washed with brine, dried over Na₂SO₄, concentrated to give the titled compound (crude, 100%). MS: M/e 376 (M+1)⁺.

### Step C: 7-((2R,5R)-2-(methoxymethyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of 7-((2R,5R)-2-(methoxymethyl)-5-methylpiperazin-1-yl)-4-methyl-2-(tetrahydro-2H-pyran-2-yl)-2,4-dihydro-5H-pyrazolo[4,3-b]pyridin-5-one (crude, 0.165 mmol), 1-(quinoxalin-6-yl)ethan-1-ol (86.1 mg, 0.495 mmol), (cyanomethyl)trimethylphosphonium iodide (120 mg, 0.495 mmol) and DIPEA (213 mg, 1.65 mmol) in CH₃CN (4 mL) was stirred at 100° C. overnight in a sealed tube. The reaction mixture was poured into H₂O (10 mL), extracted with EA (10 mL×2). The combined organic layers were washed with brine, dried over Na₂SO₄ and concentrated to dryness. The resulting residue was purified by Pre-TLC (CH₂Cl₂:MeOH=10:1) to give the titled compound (30 mg, 34%). MS: M/e 532 (M+1)⁺.

### Step D: 2-(7-((2R,5R)-2-(methoxymethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a stirred solution of 7-((2R,5R)-2-(methoxymethyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (30 mg, 0.056 mmol) in MeOH (5 mL) was added HCl (2 mL, 4.0 M in 1,4-dioxane). Then the mixture was stirred overnight. The reaction mixture was concentrated to give the residue, which was dissolved in DMF/H₂O (3 mL/1 mL) and K₂CO₃ (15.2 mg, 0.11 mmol) was added, followed by 2-iodoacetonitrile (18.8 mg, 0.11 mmol). Then the mixture was stirred overnight. The mixture was poured into H₂O (10 mL), extracted with EA (10 mL×3). The combined organic layers were washed with brine, dried over Na₂SO₄ and concentrated to dryness. The resulting residue was purified by Pre-TLC (CH₂Cl₂:MeOH=10:1) to give the titled compound (12 mg). ¹H NMR (400 MHz, CD3OD) δ 8.90-8.85 (m, 2H), 8.14-7.97 (m, 3H), 7.94 (d, J=1.6 Hz, 1H), 5.61 (d, J=12.8 Hz, 1H), 5.47 (d, J=8.4 Hz, 2H), 4.05-3.95 (m, 1H), 3.86-3.57 (m, 3H), 3.44 (s, 3H), 3.34 (s, 2H), 3.22 (d, J=12.0 Hz, 1H), 3.12 (s, 1H), 3.04-2.76 (m, 3H), 2.47 (d, J=12.4 Hz, 1H), 1.49-1.41 (m, 3H), 1.23-1.04 (m, 3H) ppm. MS: M/e 487 (M+1)⁺

### Compound A144: 2-(7-((2S,5R)-2,5-diethyl-4-(1-(1-ethyl-1H-benzo[d]imidazol-2-yl)ethyl)piperazin-1-yl)-4-methyl-5-oxo-4,5-dihydro-2H-pyrazolo[4,3-b]pyridin-2-yl)acetonitrile

### Step A: 1-(1-ethyl-1H-benzo[d]imidazol-2-yl)ethan-1-ol

To a solution of N¹-ethylbenzene-1,2-diamine (500 mg, 3.67 mmol), 2-hydroxypropanoic acid (2 mL, 85% in Water) in MeOH (30 mL) was added concentrated HCl (3 mL). The mixture solution was refluxed for 12 hours. Then the mixture was basified by Na₂CO₃ (4M) to pH ˜10 and extracted with DCM (50 mL×3). The combined organic layers were washed with brine (20 mL×3), dried over Na₂SO₄ and concentrated under reduced pressure. The resulting residue was purified by flash column chromatography to give the titled compound (100 mg, 14%). MS: M/e 191 (M+1)⁺.

### Step B: 2-(7-((2S,5R)-2,5-diethyl-4-(1-(1-ethyl-1H-benzo[d]imidazol-2-yl)ethyl)piperazin-1-yl)-4-methyl-5-oxo-4,5-dihydro-2H-pyrazolo[4,3-b]pyridin-2-yl)acetonitrile

To a solution of 1-(1-ethyl-1H-benzo[d]imidazol-2-yl)ethan-1-ol (38 mg, 0.2 mmol), Intermediate 5 (33 mg, 0.1 mmol) and (cyanomethyl)trimethylphosphonium iodide (49 mg, 0.2 mmol) in CH₃CN (2 mL) was added DIPEA (65 mg, 0.5 mmol). The mixture solution was degassed 3 times under N₂ atmosphere. Then the mixture solution was stirred at 105° C. for 24 hours. The reaction mixture was quenched with saturated NH₄Cl (20 mL) at RT and extracted with EA (30 mL×2). The combined organic layers were dried over Na₂SO₄ and concentrated under reduced pressure. The resulting residue was purified by flash column chromatography to give the titled compound (3 mg, 6%). ¹H NMR (400 MHz, CD₃OD) δ 7.93-7.92 (m, 1H), 7.51 (m, 1H), 7.30 (m, 1H), 7.28-7.24 (m, 2H), 5.56-5.33 (m, 3H), 4.88 (m, 1H), 4.47-4.37 (m, 2H), 3.48 (s, 3H), 3.22-2.99 (m, 1H), 2.78 (m, 1H), 2.53-2.44 (m, 1H), 2.17-2.02 (m, 2H), 1.63-1.54 (m, 3H), 1.52-1.46 (m, 3H), 1.46-1.32 (m, 3H), 1.28-0.96 (m, 2H), 0.89-0.62 (m, 6H) ppm. MS: M/e 501 (M+1)⁺.

### Compound A145: 2-(7-((2S,5R)-2-(2-hydroxyethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile cetonitrile

### Step A: tert-butyl (2R,5S)-5-(2-((tert-butyldimethylsilyl)oxy)ethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate

To a solution of Intermediate 2 (415 mg, 1.09 mmol) in CH₃CN (10 mL) and was added tert-butyl (2R,5S)-5-(2-((tert-butyldimethylsilyl)oxy)ethyl)-2-methylpiperazine-1-carboxylate (300 mg, 0.84 mmol) and DIPEA (324 mg, 2.51 mmol). The resulting mixture was stirred at 100° C. overnight. The reaction solvent was removed under reduce pressure. The resulting residue was purified by flash column chromatography (PE/EA) to give the titled compound (280 mg, 57%). MS: M/e 590 (M+1)⁺.

### Step B: 7-((2S,5R)-2-(2-hydroxyethyl)-5-methylpiperazin-1-yl)-4-methyl-2-(tetrahydro-2H-pyran-2-yl)-2,4-dihydro-5H-pyrazolo[4,3-b]pyridin-5-one

To a solution of tert-butyl (2R,5S)-5-(2-((tert-butyldimethylsilyl)oxy)ethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (200 mg, 0.169 mmol) in DCM (10 mL) were added TFA (1 mL). The resulting mixture was stirred at RT for another 4 hours. The reaction solvent was removed under reduce pressure. The resulting residue was dissolved into water. The aqueous was adjusted to pH=9 with saturated NaHCO₃ aq. and extracted with (DCM:IPA=4:1) to give the titled compound (100 mg). MS: M/e 376 (M+1)⁺.

### Step C: 7-((2S,5R)-2-(2-((tert-butyldimethylsilyl)oxy)ethyl)-5-methylpiperazin-1-yl)-4-methyl-2-(tetrahydro-2H-pyran-2-yl)-2,4-dihydro-5H-pyrazolo[4,3-b]pyridin-5-one

To a solution of 7-((2S,5R)-2-(2-hydroxyethyl)-5-methylpiperazin-1-yl)-4-methyl-2-(tetrahydro-2H-pyran-2-yl)-2,4-dihydro-5H-pyrazolo[4,3-b]pyridin-5-one (100 mg, 0.26 mmol) in DCM (30 mL) were added Et₃N (54 mg, 0.53 mmol) tert-butylchlorodimethylsilane (60 mg, 0.39 mmol). The resulting mixture was stirred at RT overnight. The reaction solvent was concentrated. The resulting residue was purified by Prep-TLC (DCM:MeOH=50:1) to give the titled compound (100 mg, 77%). MS: M/e 490 (M+1)⁺.

### Step D: 7-((2S,5R)-2-(2-((tert-butyldimethylsilyl)oxy)ethyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of 7-((2S,5R)-2-(2-((tert-butyldimethylsilyl)oxy)ethyl)-5-methylpiperazin-1-yl)-4-methyl-2-(tetrahydro-2H-pyran-2-yl)-2,4-dihydro-5H-pyrazolo[4,3-b]pyridin-5-one (100 mg, 0.204 mmol) in CH₃CN (8 mL) and was added 1-(quinoxalin-6-yl)ethan-1-ol (47 mg, 0.26 mmol), (cyanomethyl)trimethylphosphonium iodide (150 mg, 0.61 mmol) and DIPEA (263 mg, 2.04 mmol). The resulting mixture was stirred at 100° C. overnight. The reaction solvent was removed under reduce pressure. The resulting residue was purified by Prep-TLC (DCM:MeOH=50:1) to give the titled compound (80 mg, 61%). MS: M/e 646 (M+1)⁺.

### Step E: 7-((2S,5R)-2-(2-hydroxyethyl)-5-methyl-4-(1-(quinoxalin-6-yl)ethyl)piperazin-1-yl)-4-methyl-2,4-dihydro-5H-pyrazolo[4,3-b]pyridin-5-one

To a solution of 7-((2S,5R)-2-(2-((tert-butyldimethylsilyl)oxy)ethyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (80 mg, 0.12 mmol) in MeOH (1 mL) was added HCl (2 mL, 4 M in 1,4-dioxane). The resulting mixture was stirred at RT for 3 hours. The reaction solvent was removed under reduce pressure to give the titled compound (30 mg) which was used to the next step directly without further purification. MS: M/e 448 (M+1)⁺.

### Step F: 2-(7-((2S,5R)-2-(2-hydroxyethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 7-((2S,5R)-2-(2-hydroxyethyl)-5-methyl-4-(1-(quinoxalin-6-yl)ethyl)piperazin-1-yl)-4-methyl-2,4-dihydro-5H-pyrazolo[4,3-b]pyridin-5-one (30 mg, 0.067 mmol) crude in DMF (1.5 mL)/water (0.2 mL) were added K₂CO₃ (46 mg, 0.33 mmol). The resulting mixture was stirred at RT for 10 min, then added 2-iodoacetonitrile (56 mg, 0.33 mmol). The resulting mixture was stirred at RT for 1.5 hours. The reaction mixture was quenched by saturated NaCl aq. and extracted with EA. The organic layer was removed under reduce pressure. The resulting residue was purified Prep-HPLC(Method A) to give the titled compound (2 mg, 6%). ¹H NMR (400 MHz, CD₃OD) δ 8.98-8.85 (m, 2H), 8.20-7.97 (m, 3H), 7.93 (s, 1H), 5.66 (s, 1H), 5.46 (s, 2H), 4.02-3.75 (m, 1H), 3.74-3.58 (m, 2H), 3.54-3.33 (m, 5H), 3.25-3.01 (m, 1H), 2.98-2.65 (m, 2H), 2.49-1.60 (m, 3H), 1.45 (t, J=6.2 Hz, 3H), 1.25-1.00 (m, 3H) ppm. MS: M/e 487 (M+1)⁺.

### Compound A149: 2-(7-((2S,5R)-4-(2,3-dihydro-1H-inden-1-yl)-2,5-diethylpiperazin-1-yl)-4-methyl-5-oxo-4,5-dihydro-2H-pyrazolo[4,3-b]pyridin-2-yl)acetonitrile

### Step A: 2-(7-((2S,5R)-4-(2,3-dihydro-1H-inden-1-yl)-2,5-diethylpiperazin-1-yl)-4-methyl-5-oxo-4,5-dihydro-2H-pyrazolo[4,3-b]pyridin-2-yl)acetonitrile

To a solution of Intermediate 5 (45 mg, 0.137 mmol), 2,3-dihydro-1H-inden-1-ol (37 mg, 0.274 mmol) and (cyanomethyl)trimethylphosphonium iodide (67 mg, 0.274 mmol) in CH₃CN (1 mL) was added DIPEA (88 mg, 0.685 mmol). The reaction mixture was degassed with N₂ (3 times) and then stirred at 105° C. for 24 hours. The reaction mixture was quenched with saturated NH₄Cl (20 mL) at room temperature and extracted with EA (35 mL×2). The combined organic layers were dried over Na₂SO₄ and concentrated under reduced pressure, to obtain Compound 149. The resulting residue containing Compound 149 was purified by Prep-HPLC(Method A) to give the titled Compound A149a (5 mg) and Compound A149b (4 mg).

Compound A149a: ¹H NMR (400 MHz, CD₃OD) δ 7.96 (s, 1H), 7.42-7.41 (m, 1H), 729-7.27 (m, 3H), 5.52-5.42 (m, 4H), 4.71 (s, 1H), 4.11 (s, 1H), 3.73-3.67 (m, 1H), 3.42 (s, 3H), 3.25-2.87 (m, 5H), 2.34-2.26 (m, 2H), 1.71-1.48 (m, 4H), 1.00 (t, J=4 Hz, 3H), 0.80 (t, J=4 Hz, 3H) ppm. MS: M/e 445 (M+1)⁺.

Compound A149b: ¹H NMR (400 MHz, CD₃OD) δ 7.94 (s, 1H), 7.41 (s, 1H), 7.23-7.17 (m, 3H), 5.57 (s, 1H), 5.48 (s, 2H), 4.49-4.45 (m, 1H), 3.44-3.43 (m, 1H), 3.37 (s, 3H), 3.30-2.23 (m, 2H), 3.13-2.81 (m, 4H), 2.43-2.20 (m, 3H), 1.99-1.95 (m, 1H), 1.78-1.74 (m, 2H), 1.60-1.56 (m, 1H), 0.97 (t, J=8 Hz, 3H), 0.75 (t, J=8 Hz, 3H) ppm. MS: M/e 445 (M+1)⁺.

### Compound A152: 2-(7-((2S,5R)-2,5-diethyl-4-(1-(4-fluoro-2-(hydroxymethyl)phenyl)ethyl)piperazin-1-yl)-4-methyl-5-oxo-4,5-dihydro-2H-pyrazolo[4,3-b]pyridin-2-yl)acetonitrile

### Step A: ((2-bromo-5-fluorobenzyl)oxy)(tert-butyl)dimethylsilane

To a solution of (2-bromo-5-fluorophenyl)methanol (3 g, 14.6 mmol) in DCM (40 mL) was added TBSCl (4.3 g, 29.2 mmol) and Et₃N (5.9 g, 58.5 mmol). The resulting mixture was stirred at 40° C. for 8 hours. The reaction mixture was washed with water and concentrated. The resulting residue was purified by flash column chromatography (PE/EA) to give the titled compound (4.2 g, 90%). ¹H NMR (400 MHz, CDCl3) δ 7.44 (s, 1H), 7.18 (s, 1H), 6.98 (s, 1H), 4.62 (s, 2H), 0.89 (s, 9H), 0.09 (s, 6H) ppm.

### Step B: 1-(2-(((tert-butyldimethylsilyl)oxy)methyl)-4-fluorophenyl)ethan-1-ol

To a solution of ((2-bromo-5-fluorobenzyl)oxy)(tert-butyl)dimethylsilane(3 g, 9.4 mmol) in THF (30 mL) was added n-BuLi (7.9 mL, 1.6M, 12.7 mmol) at −78° C. The resulting mixture was stirred at −78° C. for another 1 hour. To the above reaction mixture, acetaldehyde (1.6 g, 47 mmol) was added dropwise. The reaction mixture was stirred at −70° C. for another 3 hours. The reaction mixture was quenched by saturated NH₄Cl aq. and extracted by EA. The organic layer was concentrated. The resulting residue was purified by flash column chromatography (PE/EA) to give the titled compound (760 mg). MS: M/e 267 (M+1-18)⁺.

### Step C: 7-((2S,5R)-4-(1-(2-(((tert-butyldimethylsilyl)oxy)methyl)-4-fluorophenyl)ethyl)-2,5-diethylpiperazin-1-yl)-4-methyl-2-(tetrahydro-2H-pyran-2-yl)-2,4-dihydro-5H-pyrazolo[4,3-b]pyridin-5-one

To a solution of 1-(2-(((tert-butyldimethylsilyl)oxy)methyl)-4-fluorophenyl)ethan-1-ol (171 mg, 0.6 mmol) in CH₃CN (10 mL) and was added Intermediate 3 (150 mg, 0.4 mmol), (cyanomethyl)trimethylphosphonium iodide (390 mg, 1.6 mmol) and DIPEA (516 mg, 4 mmol). The resulting mixture was stirred at 100° C. overnight. The reaction mixture was removed under reduce pressure. The resulting residue was dissolved into EA. The organic layer was washed with water, brine, dried over Na₂SO₄ and concentrated to give the titled compound (crude) which was used directly to next step without further purification.

### Step D: 7-((2S,5R)-2,5-diethyl-4-(1-(4-fluoro-2-(hydroxymethyl)phenyl)ethyl)piperazin-1-yl)-4-methyl-2-(tetrahydro-2H-pyran-2-yl)-2,4-dihydro-5H-pyrazolo[4,3-b]pyridin-5-one

To a solution of 7-((2S,5R)-4-(1-(2-(((tert-butyldimethylsilyl)oxy)methyl)-4-fluorophenyl)ethyl)-2,5-diethylpiperazin-1-yl)-4-methyl-2-(tetrahydro-2H-pyran-2-yl)-2,4-dihydro-5H-pyrazolo[4,3-b]pyridin-5-one (crude, 0.4 mmol) in THF (5 mL) was added TBAF (0.6 mL, 0.6 mmol). The resulting mixture was stirred at RT for 2 hours. The reaction mixture was concentrated. The resulting residue was purified by Prep-TLC (DCM:MeOH=25:1) to give the titled compound (60 mg, two steps 28%). MS: M/e 526 (M+1)⁺.

### Step E: 7-((2S,5R)-2,5-diethyl-4-(1-(4-fluoro-2-(hydroxymethyl)phenyl)ethyl)piperazin-1-yl)-4-methyl-2,4-dihydro-5H-pyrazolo[4,3-b]pyridin-5-one

To a solution of 7-((2S,5R)-2,5-diethyl-4-(1-(4-fluoro-2-(hydroxymethyl)phenyl)ethyl)piperazin-1-yl)-4-methyl-2-(tetrahydro-2H-pyran-2-yl)-2,4-dihydro-5H-pyrazolo[4,3-b]pyridin-5-one (60 mg, 0.11 mmol) in MeOH (1 mL) was added HCl (2 mL, 4 M in 1,4-dioxane). The resulting mixture was stirred at RT for 2 hours. The reaction solvent was removed under vacuum to give the titled compound (crude) which was used to next step directly without further purification.

### Step F: 2-(7-((2S,5R)-2,5-diethyl-4-(1-(4-fluoro-2-(hydroxymethyl)phenyl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-diethyl-4-(1-(4-fluoro-2-(hydroxymethyl)phenyl)ethyl)piperazin-1-yl)-4-methyl-2,4-dihydro-5H-pyrazolo[4,3-b]pyridin-5-one (60 mg, 0.136 mmol) crude in DMF (3 mL)/water (0.35 mL) were added K₂CO₃ (94 mg, 0.68 mmol) and 2-iodoacetonitrile (114 mg, 0.68 mmol). The resulting mixture was stirred at RT for 1 hours. The reaction mixture was quenched by saturated NaCl aq. and extracted with EA. The organic layer was removed under vacuum. The resulting residue was purified by Prep-TLC (DCM:MeOH=15:1), then purified by Prep-HPLC (Method A) to give the titled compound (2 mg, 3%). ¹H NMR (400 MHz, CD₃OD) δ 7.92 (s, 1H), 7.58-7.15 (m, 2H), 7.08-6.90 (m, 1H), 5.61-5.49 (m, 1H), 5.47 (s, 2H), 4.75-4.55 (m, 2H), 4.20-3.99 (m, 1H), 3.60-3.33 (m, 4H), 3.25-2.70 (m, 2H), 2.70-2.22 (m, 2H), 2.20-1.75 (m, 2H), 1.74-1.41 (m, 3H), 1.40-1.25 (m, 3H), 1.24-0.80 (m, 3H), 0.75-0.53 (m, 3H) ppm. MS: M/e 481 (M+1)⁺.

### Compound A154: 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-d]pyrimidin-2-yl)acetonitrile

### Step A: 7-((2S,5R)-2,5-dimethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one

To a solution of Intermediate 9 (108 mg, 0.4 mmol) in CH₃CN (4 mL) was added DIPEA (103 mg, 0.8 mmol), followed by POCl₃ (122 mg, 0.8 mmol) and a drop of DMF. The reaction was stirred at 80° C. for 5 hours. The reaction was cooled to room temperature, diluted with saturated NaHCO₃ solution, extracted with EA (60 mL×2), washed with brine, dried over Na₂SO₄, filtered, and concentrated to dryness. The resulting residue was dissolved in CH₃CN (3 mL), Intermediate 1 (50 mg, 0.2 mmol) and DIPEA (50 mg, 0.4 mmol) were added. The resulting mixture was heated at 80° C. for 15 hours. The reaction mixture was quenched with water, extracted with EA (60 mL×3), washed with brine, dried over Na₂SO₄, filtered, and concentrated to dryness. The resulting residue was purified by Prep-TLC (DCM:MeOH=15:1) to give titled compound (40 mg, 20%). MS: M/e 503 (M+1)⁺.

### Step B: 7-((2S,5R)-2,5-dimethyl-4-(1-(quinoxalin-6-yl)ethyl)piperazin-1-yl)-4-methyl-2,4-dihydro-5H-pyrazolo[4,3-d]pyrimidin-5-one

To a solution of 7-((2S,5R)-2,5-dimethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one (40 mg, 0.079 mmol) in MeOH (4 mL) was added a solution of 4M HCl in 1,4-dioxane (4 mL). The resulting mixture was stirred at room temperature for 56 hours. The mixture was concentrated under reduced pressure, diluted with water/DCM, basified with saturated NaHCO₃ solution to pH 7-8, extracted with DCM:IPA (4:1, 40 mL×3). The combined organic layers were washed with brine, dried over Na₂SO₄, filtered and concentrated to dryness. The resulting residue was purified by Prep-TLC (DCM:MeOH=10:1) to give titled compound (21 mg, 63%). MS: M/e 419 (M+1)⁺.

### Step C: 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-d]pyrimidin-2-yl)acetonitrile

To a solution of 7-((2S,5R)-2,5-dimethyl-4-(1-(quinoxalin-6-yl)ethyl)piperazin-1-yl)-4-methyl-2,4-dihydro-5H-pyrazolo[4,3-d]pyrimidin-5-one (21 mg, 0.05 mmol) and K₂CO₃ (14 mg, 0.1 mmol) in DMF (2 mL) was added 2-iodoacetonitrile (13 mg, 0.075 mmol). The reaction was stirred at room temperature overnight. The reaction mixture was diluted with water, extracted with EA (60 mL×2), washed with brine, dried over Na₂SO₄, filtered, and concentrated to dryness. The resulting residue was purified by Prep-HPLC to give the titled compound (6 mg, 26%). ¹HNMR (400 MHz, CD₃OD) δ 8.92-8.84 (m, 2H), 8.16-8.00 (m, 3H), 7.96-7.86 (m, 1H), 5.72-5.30 (m, 3H), 5.20-5.00 (m, 0.5H), 4.80-4.58 (m, 0.5H), 4.01-3.89 (m, 1H), 3.86-3.65 (m, 1H), 3.41 (s, 3H), 3.19-2.69 (m, 2.5H), 2.31-2.20 (m, 0.5H), 1.65-1.24 (m, 6H), 1.16-1.09 (m, 1H), 1.02-0.92 (m, 2H) ppm. MS: M/e 458 (M+1)⁺.

### Compound A156: 2-(cyanomethyl)-7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridine-6-carbonitrile

### Step A: 2-(6-bromo-7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 2-(7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile (70 mg, 0.15 mmol) in CH₃CN (5 mL) were added NBS (32 mg, 0.18 mmol) slowly at room temperature. The resulting mixture was stirred at room temperature for another 2 hours. The reaction mixture was diluted with EA (20 mL) and washed with water (10 mL×2). The organic layers were concentrated to dryness. The resulting residue was purified by pre-TLC (DCM:MeOH=15:1) to give the titled compound (50 mg, 61%). MS: M/e 549 (M+1)⁺.

### Step B: 2-(cyanomethyl)-7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridine-6-carbonitrile

A mixture of 2-(6-bromo-7-((2S,5R)-5-ethyl-2-methyl-4-(1-(quinoxalin-6-yl)ethyl) piperazin-1-yl)-4-methyl-5-oxo-4,5-dihydro-2H-pyrazolo[4,3-b]pyridin-2-yl)acetonitrile (50 mg, 0.09 mmol), Pd(PPh₃)₄ (23 mg, 0.02 mmol) and Zn(CN)₂ (21 mg, 0.18 mmol) in DMF (2 mL) was heated to 100° C. for overnight under N₂ atmosphere. The reaction mixture was diluted with EA (10 mL) and washed with brine (5 mL×2). The organic layers were concentrated. The resulting residue was purified by pre-TLC (DCM:MeOH=15:1) to give the titled compound (17 mg, 38%). ¹H NMR (400 MHz, CD₃OD−δ 8.88-8.87 (m, 2H), 8.15-8.07 (m, 3H), 8.01-7.99 (m, 1H), 5.51-5.49 (m, 1H), 5.48 (m, 2H), 4.10-4.08 (m, 1H), 3.98-3.95 (m, 1H), 3.45-3.43 (m, 3H), 3.20-3.13 (m, 1H), 3.01-2.08 (m, 1H), 2.55-2.53 (m, 1H), 2.32-2.29 (m, 1H), 1.47-1.45 (m, 3H), 1.40-1.28 (m, 5H), 0.97-0.93 (m, 2H), 0.63-0.60 (m, 2H) ppm. MS: M/e 496 (M+1)⁺.

### Compound A158: 2-((2S,5R)-1-(2-(cyanomethyl)-4-methyl-5-oxo-4,5-dihydro-2H-pyrazolo[4,3-b]pyridin-7-yl)-5-methyl-4-(1-(quinoxalin-6-yl)ethyl)piperazin-2-yl)acetonitrile

### Step A: tert-butyl (2R,5R)-2-methyl-4-(4-methyl-5-oxo-2-(tetrahydro-2H-pyran-2-yl)-4,5-dihydro-2H-pyrazolo[4,3-b]pyridin-7-yl)-5-(((methylsulfonyl)oxy)methyl)piperazine-1-carboxylate

To a stirred solution of tert-butyl (2R,5R)-5-(hydroxymethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (112 mg, 0.243 mmol) in CH₂Cl₂ (10 mL) was added Et₃N (50 mg, 0.486 mmol), then MsCl (55.6 mg, 0.486 mmol) was added. After the addition, the reaction mixture was stirred at room temperature overnight. The reaction mixture was poured into H₂O (20 mL), extracted with CH₂Cl₂ (20 mL). The combined organic layers were dried over Na₂SO₄ and concentrated to dryness. The resulting residue was purified by Pre-TLC (CH₂Cl₂:MeOH=10:1) to give the titled compound (50 mg, 38%). MS: M/e 540 (M+1)⁺.

### Step B: tert-butyl (2R,5S)-5-(cyanomethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate

To a stirred solution of tert-butyl (2R,5R)-2-methyl-4-(4-methyl-5-oxo-2-(tetrahydro-2H-pyran-2-yl)-4,5-dihydro-2H-pyrazolo[4,3-b]pyridin-7-yl)-5-(((methylsulfonyl)oxy)methyl)piperazine-1-carboxylate (50 mg, 0.09 mmol) in CH₃CN (5 mL) was added Cs₂CO₃ (88 mg, 0.27 mmol), followed by TMSCN (18.4 mg, 0.18 mmol). After the addition, the reaction mixture was stirred at 70° C. for 5 days. The reaction mixture was poured into H₂O (10 mL), extracted with EA (10 mL×3). The combined organic layers were washed with brine, dried over Na₂SO₄, concentrated. The resulting residue was purified by flash column chromatography to give the titled compound (30 mg, 71%). MS: M/e 471 (M+1)⁺.

### Step C: 2-((2S,5R)-5-methyl-1-(4-methyl-5-oxo-2-(tetrahydro-2H-pyran-2-yl)-4,5-dihydro-2H-pyrazolo[4,3-b]pyridin-7-yl)piperazin-2-yl)acetonitrile

To a stirred solution of tert-butyl (2R,5S)-5-(cyanomethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (30 mg, 0.064 mmol) in CH₂Cl₂ (5 mL) was added TFA (1 mL). Then the mixture was stirred for 4 hours. The reaction mixture was basified with saturated Na₂CO₃ aq., then extracted with CH₂Cl₂ (10 mL×3). The combined organic layers were washed with brine, dried over Na₂SO₄, concentrated to give the titled compound (23 mg, 97%). MS: M/e 371 (M+1)⁺.

### Step D: 2-((2S,5R)-5-methyl-1-(4-methyl-5-oxo-2-(tetrahydro-2H-pyran-2-yl)-4,5-dihydro-2H-pyrazolo[4,3-b]pyridin-7-yl)-4-(1-(quinoxalin-6-yl)ethyl)piperazin-2-yl)acetonitrile

A mixture of 2-((2S,5R)-5-methyl-1-(4-methyl-5-oxo-2-(tetrahydro-2H-pyran-2-yl)-4,5-dihydro-2H-pyrazolo[4,3-b]pyridin-7-yl)piperazin-2-yl)acetonitrile (23 mg, 0.062 mmol), 1-(quinoxalin-6-yl)ethan-1-ol (32.4 mg, 0.186 mmol), (cyanomethyl)trimethylphosphonium iodide (45.2 mg, 0.186 mmol) and DIPEA (80 mg, 0.62 mmol) in CH₃CN (3 mL) was stirred at 100° C. overnight in a sealed tube. The reaction mixture was poured into H₂O (10 mL), extracted with EA (10 mL×2). The combined organic layers were washed with brine, dried over Na₂SO₄ and concentrated to dryness. The resulting residue was purified by Pre-TLC (CH₂Cl₂:MeOH=10:1) to give the titled compound (15 mg, 46%). MS: M/e 527 (M+1)⁺.

### Step E: 2-((2S,5R)-1-(2-(cyanomethyl)-4-methyl-5-oxo-4,5-dihydro-2H-pyrazolo[4,3-b]pyridin-7-yl)-5-methyl-4-(1-(quinoxalin-6-yl)ethyl)piperazin-2-yl)acetonitrile

To a stirred solution of 2-((2S,5R)-5-methyl-1-(4-methyl-5-oxo-2-(tetrahydro-2H-pyran-2-yl)-4,5-dihydro-2H-pyrazolo[4,3-b]pyridin-7-yl)-4-(1-(quinoxalin-6-yl)ethyl)piperazin-2-yl)acetonitrile (15 mg, 0.028 mmol) in MeOH (3 mL) was added HCl (1 mL, 4.0 M in 1,4-dioxane). Then the mixture was stirred at room temperature overnight. The reaction mixture was concentrated to give the residue, which was dissolved in DMF/H₂O (3 mL/1 mL) and K₂CO₃ (11.6 mg, 0.084 mmol) was added, followed by 2-iodoacetonitrile (9.35 mg, 0.056 mmol). Then the mixture was stirred for 2 hours. The mixture was poured into H₂O (10 mL), extracted with EA (10 mL×3). The combined organic layers were washed with brine, dried over Na₂SO₄ and concentrated to dryness. The resulting residue was purified by Prep-TLC (CH₂Cl₂:MeOH=10:1) to give the titled compound (1.3 mg). ¹H NMR (400 MHz, CD3OD) δ 8.91-8.84 (m, 2H), 8.16-7.92 (m, 4H), 5.72-5.62 (m, 1H), 5.53-5.46 (m, 2H), 4.05-3.96 (M, 0.5H), 3.91-3.72 (m, 1.5H), 3.67-3.57 (m, 1H), 3.45 (s, 3H), 3.28-2.84 (m, 5H), 2.39 (d, J=12.4 Hz, 0.6H), 2.06-1.98 (m, 0.4H), 1.51-1.44 (m, 3H), 1.23-1.04 (m, 3H) ppm. MS: M/e 482 (M+1)⁺.

### Compound A159: 2-(7-((2S,5R)-4-(1-(2,3-dihydro-[1,4]dioxino[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile

### Step A: 2-((2-chloro-6-iodopyridin-3-yl)oxy)ethan-1-ol

To a solution of 2-chloro-6-iodopyridin-3-ol (2.55 g, 10 mmol) in DMF (5 mL) was added 1,3-dioxolan-2-one (1.76 g, 20 mmol) and K₂CO₃ (2.78 g, 20 mmol). The reaction mixture was stirred under nitrogen protection at 150° C. for 3 hours. The reaction mixture was poured into H₂O and extracted with EA. The combined organic layers were dried over Na₂SO₄, filtered and concentrated to dryness. The resulting residue was purified by flash column chromatography to give the titled compound (2.1 g, 70%). MS: m/e 300 (M+1)⁺.

### Step B: 6-iodo-2,3-dihydro-[1,4]dioxino[2,3-b]pyridine

To a solution of 2-((2-chloro-6-iodopyridin-3-yl)oxy)ethan-1-ol (2 g, 6.7 mmol) in toluene (15 mL) was added TBAF (1M, 0.5 mL) and KOH (560 mg, 10 mmol). The resulting mixture was stirred at 110° C. overnight. The reaction solvent was concentrated in vacuo. The resulting residue was dissolved in H₂O. The aqueous was extracted by EA. The organic layer was dried over Na₂SO₄, filtered, and concentrated to dryness. The resulting residue was purified by flash column chromatography to give the titled compound (500 mg, 29%). MS: m/e 264 (M+1)⁺.

### Step C: 1-(2,3-dihydro-[1,4]dioxino[2,3-b]pyridin-6-yl)ethan-1-one

To a solution of 6-iodo-2,3-dihydro-[1,4]dioxino[2,3-b]pyridine (500 mg, 2 mmol) in toluene (15 mL) was added Pd(PPh₃)₂Cl₂ (70 mg, 0.1 mmol) and ethyl tributylstannanecarboxylate (1.08 g, 3 mmol). The resulting mixture was stirred at 110° C. under N₂ atmosphere overnight. The reaction solvent was concentrated in vacuo. The resulting residue was dissolved in H₂O. The aqueous was extracted by EA. The organic layer was dried over Na₂SO₄, filtered, and concentrated to dryness. The resulting residue was purified by flash column chromatography to give the titled compound (300 mg, 84%). MS: m/e 180 (M+1)⁺.

### Step D: 1-(2,3-dihydro-[1,4]dioxino[2,3-b]pyridin-6-yl)ethan-1-ol

To a solution of 1-(2,3-dihydro-[1,4]dioxino[2,3-b]pyridin-6-yl)ethan-1-one (480 mg, 2.67 mmol) in EtOH (15 mL) was added NaBH₄ (405 mg, 11 mmol). The resulting mixture was stirred at RT overnight. The reaction solvent was concentrated in vacuo. The resulting residue was dissolved in H₂O. The aqueous was extracted by EA. The organic layer was dried over Na₂SO₄, filtered, and concentrated to dryness. The resulting residue was purified by flash column chromatography to give the titled compound (400 mg, 83%). MS: m/e 182 (M+1)⁺.

### Step E: 2-(7-((2S,5R)-4-(1-(2,3-dihydro-[1,4]dioxino[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile

To a solution of Intermediate 5 (40 mg, 0.12 mmol) in MeCN (5 mL) was added 1-(2,3-dihydro-[1,4]dioxino[2,3-b]pyridin-6-yl)ethan-1-ol (50 mg, 0.28 mmol), (cyanomethyl)trimethylphosphonium (120 mg, 0.5 mmol) and DIPEA (1.29 mg, 1 mmol). The reaction mixture was sealed and stirred under nitrogen protection at 105° C. for 16 hours. The reaction solvent was concentrated in vacuo. The resulting residue was dissolved in H₂O. The aqueous was extracted by EA. The organic layer was dried over Na₂SO₄, filtered, and concentrated to dryness. The resulting residue was purified by flash column chromatography to give the titled compound (1.37 mg, 2.3%). ¹H NMR (400 MHz, CD₃OD) δ 7.95 (s, 1H), 7.31 (s, 1H), 7.11 (s, 1H), 5.56 (s, 1H), 5.48 (s, 2H), 4.46 (s, 2H), 4.29 (s, 2H), 3.73 (d, J=10.2 Hz, 1H), 3.46 (d, J=14.7 Hz, 4H), 3.13 (s, 1H), 3.00-2.68 (m, 3H), 2.09-1.94 (m, 1H), 1.68-1.50 (m, 3H), 1.30 (s, 3H), 1.20-1.05 (m, 1H), 0.96 (s, 3H), 0.88-0.80 (m, 1H), 0.79-0.65 (m, 2H) ppm. MS: m/e 492 (M+1)⁺.

### Compound A161: 2-(7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: tert-butyl (3R)-3-ethyl-4-(1-(quinoxalin-6-yl)ethyl)piperazine-1-carboxylate

A mixture of tert-butyl (R)-3-ethylpiperazine-1-carboxylate (2 g, 9.3 mmol), 1-(quinoxalin-6-yl)ethan-1-ol (2.4 g, 13.9 mmol), (cyanomethyl)trimethylphosphonium iodide (4.5 g, 18.6 mmol) and DIPEA (6 g, 46.5 mmol) in CH₃CN (10 mL) was heated to 105° C. for overnight under N₂ atmosphere. The reaction solvent was removed under vacuum. The resulting residue was purified by flash column chromatography (DCM:MeOH=100:1) to give the titled compound (1.8 g, 53%). MS: M/e 371 (M+1)⁺.

### Step B: 6-(1-((R)-2-ethylpiperazin-1-yl)ethyl)quinoxaline

To a solution of tert-butyl (2S,5R)-2,5-diethyl-4-(1-(4-fluoro-2-methoxyphenyl)ethyl)piperazine-1-carboxylate (1.8 g, 4.9 mmol) in DCM (20 mL) were added TFA (4 mL) at room temperature. The resulting mixture was stirred at room temperature for another 3 hours. The reaction solvent was removed under vacuum. The resulting residue was dissolved in DCM (20 mL). The organic layer was washed with aq·NaOH and concentrated to give the titled compound (1.2 g) which was used to the next step directly without further purification. MS: M/e 271 (M+1)⁺.

### Step C: 7-((3R)-3-ethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of Intermediate 2 (1.6 g, 4.4 mmol), 6-(1-((R)-2-ethylpiperazin-1-yl)ethyl)quinoxaline (1.2 g, 4.4 mmol) and DIPEA (2.8 g, 22 mmol) in CH₃CN was heated to 105° C. for overnight under N₂ atmosphere. The reaction solvent was removed under vacuum. The resulting residue was purified by flash column chromatography (DCM:MeOH=20:1) to give the titled compound (720 mg, 33%). MS: M/e 502 (M+1)⁺.

### Step D: 7-((3R)-3-ethyl-4-(1-(quinoxalin-6-yl)ethyl)piperazin-1-yl)-4-methyl-2,4-dihydro-5H-pyrazolo[4,3-b]pyridin-5-one

To a solution of 7-((3R)-3-ethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (720 mg, 1.4 mmol) in MeOH (5 mL) was added HCl dioxane solution (3.6 mL, 14 mmol, 4M) at room temperature. The resulting mixture was stirred at room temperature for another 2 hours. The reaction solvent was removed under vacuum. The resulting residue was dissolved in DCM (20 mL). The organic layer was washed with aq·NaOH and concentrated. The resulting residue was further purified by flash column chromatography (DCM:MeOH=10:1) to give the titled compound (380 mg, 65%). MS: M/e 418 (M+1)⁺.

### Step E: 2-(7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 7-((3R)-3-ethyl-4-(1-(quinoxalin-6-yl)ethyl)piperazin-1-yl)-4-methyl-2,4-dihydro-5H-pyrazolo[4,3-b]pyridin-5-one (380 mg, 0.91 mmol) and K₂CO₃ (252 mg, 1.82 mmol) in DMF (5 mL) were added 2-iodoacetonitrile (228 mg, 1.41 mmol). The resulting mixture was stirred at room temperature overnight. The reaction mixture was diluted with EA (20 mL) and washed with brine (20 mL×3). The organic layers were concentrated under reduced pressure. The resulting residue was purified by flash column chromatography (DCM:MeOH=20:1) to give the titled Compound A161 (240 mg, 85% purity). The Compound A161 (20 mg, crude) was further purified by pre-TLC (DCM:MeOH=15:1) to give the titled Compound A161a (5 mg) and Compound A161b (2 mg).

Compound A161a (the earlier peak): ¹H NMR (400 MHz, CD₃OD) δ 8.88-8.87 (m, 2H), 8.12-8.06 (m, 3H), 7.94 (s, 1H), 5.59 (s, 1H), 5.48 (s, 2H), 4.42-4.37 (m, 2H), 3.88-3.75 (m, 1H), 3.62-3.58 (m, 1H), 3.50-3.46 (m, 1H), 3.44 (s, 3H), 3.14-3.09 (m, 1H), 2.71-2.54 (m, 2H), 1.79-1.70 (m, 2H), 1.52-1.48 (m, 3H), 1.12-1.08 (m, 3H) ppm. MS: M/e 457 (M+1)⁺.

Compound A161b ((the later peak)): ¹H NMR (400 MHz, CD₃OD) δ 8.89-8.88 (m, 2H), 8.13-7.99 (m, 3H), 7.91 (s, 1H), 5.60 (s, 1H), 5.44 (s, 2H), 4.48-4.32 (m, 2H), 4.01-3.96 (m, 1H), 3.61-3.52 (m, 2H), 3.42 (s, 3H), 3.14-2.95 (m, 2H), 2.61-2.56 (m, 1H), 1.80-1.56 (m, 5H), 0.85-0.81 (m, 3H) ppm. MS: M/e 457 (M+1)⁺.

### Compound A162: 2-(6-chloro-7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: 2-(6-chloro-7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 2-(7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile (50 mg, 0.11 mmol) in CH₃CN (5 mL) were added NCS (15 mg, 0.12 mmol) at room temperature. The resulting mixture was stirred at room temperature for another 2 hours. The reaction mixture was diluted with EA (20 mL) and washed with water (10 mL×2). The organic layers were concentrated and purified by pre-TLC (DCM:MeOH=15:1) to give two isomers of Compound A162, named as Compound A162a (10 mg) and Compound A162b (7 mg).

Compound A162a (the earlier peak): ¹H NMR (400 MHz, CD₃OD) δ 8.87 (d, J=6.0 Hz, 2H), 8.12-8.10 (m, 3H), 7.97 (s, 1H), 5.49 (s, 2H), 4.38 (br s, 1H), 4.06-4.01 (m, 1H), 3.80-3.58 (m, 3H), 3.52 (s, 3H), 3.11 (br s, 1H), 2.77-2.70 (m, 1H), 2.55 (br s, 1H), 1.83-1.79 (m, 2H), 1.48 (d, J=6.4 Hz, 3H), 1.01 (t, J=7.3 Hz, 3H) ppm. MS: M/e 491 (M+1)⁺.

Compound A162b (the later peak): ¹H NMR (400 MHz, CD₃OD) δ 8.88 (d, J=6.0 Hz, 2H), 8.13-7.99 (m, 3H), 7.95 (s, 1H), 5.47 (s, 2H), 4.41 (br s, 1H), 4.06-4.01 (m, 1H), 3.80-3.56 (m, 3H), 3.50 (s, 3H), 3.23 (br s, 1H), 2.89 (br s, 1H), 2.55 (br s, 1H), 1.86-1.57 (m, 5H), 1.01 (t, J=7.3 Hz, 3H) ppm. MS: M/e 491 (M+1)⁺

### Compound A164: 2-(7-((2S,5R)-2-(2-methoxyethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: tert-butyl (2R,5S)-5-(2-hydroxyethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate

To a solution of tert-butyl (2R,5S)-5-(2-((tert-butyldimethylsilyl)oxy)ethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (80 mg, 0.136 mmol) in THF (10 mL) and was TBAF (0.2 mL, 0.203 mmol). The resulting mixture was stirred at RT for 2 hours. The reaction solvent was concentrated. The resulting residue was purified by Prep-TLC (DCM:MeOH=25:1) to give the titled compound (60 mg, 94%). MS: M/e 476 (M+1)⁺.

### Step B: tert-butyl (2R,5S)-5-(2-methoxyethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate

To a solution of tert-butyl (2R,5S)-5-(2-hydroxyethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (60 mg, 0.126 mmol) in THF (1.5 mL) were added NaH/60%(10 mg, 0.253 mmol). The resulting mixture was stirred at RT for 20 minutes. Then added CH₃I (54 mg, 0.379 mmol), the resulting mixture was stirred at RT for another 3 hours. The reaction mixture was quenched with water and extracted with EA and concentrated. The resulting residue was purified by Prep-TLC (DCM:MeOH=50:1) to give the titled compound (50 mg, 81%). MS: M/e 490.4 (M+1)⁺.

### Step C: 7-((2S,5R)-2-(2-methoxyethyl)-5-methylpiperazin-1-yl)-4-methyl-2-(tetrahydro-2H-pyran-2-yl)-2,4-dihydro-5H-pyrazolo[4,3-b]pyridin-5-one

To a solution of 7-((2S,5R)-2-(2-hydroxyethyl)-5-methylpiperazin-1-yl)-4-methyl-2-(tetrahydro-2H-pyran-2-yl)-2,4-dihydro-5H-pyrazolo[4,3-b]pyridin-5-one (50 mg, 0.1 mmol) in DCM (5 mL) were added TFA (0.4 mL). The resulting mixture was stirred at RT for 2 hours. The reaction solvent was removed under vacuum. The resulting residue was dissolved in water. The aqueous layer was extracted with (DCM:IPA=4:1). The organic layer was washed with sat. NaHCO₃ aq. and concentrated. The resulting residue was purified by Prep-TLC (DCM:MeOH=15:1) to give the titled compound (35 mg, 88%). MS: M/e 390 (M+1)⁺.

### Step D: 7-((2S,5R)-2-(2-methoxyethyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of 7-((2S,5R)-2-(2-methoxyethyl)-5-methylpiperazin-1-yl)-4-methyl-2-(tetrahydro-2H-pyran-2-yl)-2,4-dihydro-5H-pyrazolo[4,3-b]pyridin-5-one (35 mg, 0.089 mmol) in CH₃CN (8 mL) and was added 1-(quinoxalin-6-yl)ethan-1-ol (24 mg, 0.134 mmol), (cyanomethyl)trimethylphosphonium iodide (65 mg, 0.27 mmol) and DIPEA (116 mg, 0.89 mmol). The resulting mixture was stirred at 100° C. overnight. The reaction solvent was removed under reduce pressure. The resulting residue was purified by Prep-TLC (DCM:MeOH=25:1) to give the titled compound (30 mg, 60%). MS: M/e 546 (M+1)⁺.

### Step E: 7-((2S,5R)-2-(2-methoxyethyl)-5-methyl-4-(1-(quinoxalin-6-yl)ethyl)piperazin-1-yl)-4-methyl-2,4-dihydro-5H-pyrazolo[4,3-b]pyridin-5-one

To a solution of 7-((2S,5R)-2-(2-methoxyethyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (30 mg, 0.055 mmol) in MeOH (1 mL) was added HCl (3 mL, 4 M in 1,4-dioxane). The resulting mixture was stirred at RT for 2 hours. The reaction solvent was removed under vacuum to give the titled compound which was used to the next step directly without further purification. MS: M/e 462 (M+1)⁺.

### Step F: 2-(7-((2S,5R)-2-(2-methoxyethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 7-((2S,5R)-2-(2-methoxyethyl)-5-methyl-4-(1-(quinoxalin-6-yl)ethyl)piperazin-1-yl)-4-methyl-2,4-dihydro-5H-pyrazolo[4,3-b]pyridin-5-one (30 mg, 0.065 mmol) crude in DMF (2 mL)/water (0.3 mL) were added K₂CO₃ (45 mg, 0.33 mmol) and 2-iodoacetonitrile (54 mg, 0.33 mmol). The resulting mixture was stirred at RT for 1.5 hours. The reaction mixture was quenched by saturated NaCl aq. and extracted with EA. The organic layer was removed under vacuum. The resulting residue was purified by Prep-TLC (DCM:MeOH=25:1), then purified by Prep-HPLC (Method A) to give the titled compound (4.66 mg, 14%). ¹H NMR (400 MHz, CD₃OD) δ 8.98-8.80 (m, 2H), 8.20-7.94 (m, 3H), 7.92 (s, 1H), 5.65 (s, 1H), 5.47 (s, 2H), 4.02-3.75 (m, 1H), 3.74-3.60 (m, 2H), 3.54-3.33 (m, 6H), 3.29-3.15 (m, 1H), 3.14-2.70 (m, 5H), 2.47-2.00 (m, 2H), 1.58-1.30 (m, 3H), 1.25-1.00 (m, 3H) ppm. MS: M/e 501 (M+1)⁺.

### Compound A166: 2-(7-((2S,5R)-2,5-diethyl-4-(1-(1-methyl-2,3-dihydro-1H-pyrido[2,3-b][1,4]oxazin-6-yl)ethyl)piperazin-1-yl)-4-methyl-5-oxo-4,5-dihydro-2H-pyrazolo[4,3-b]pyridin-2-yl)acetonitrile

### Step A: 3-amino-6-bromopyridin-2 (1H)-one

To a solution of 6-bromo-2-methoxypyridin-3-amine (1 g, 5 mmol) in H₂O (5 mL) was added HBr (33% in water, 10 mmol). The reaction mixture was stirred at 80° C. overnight. The reaction mixture was cooled to RT and adjusted pH to 7-8 with saturated NaHCO₃ aq. to give a suspension. The filter cake was collected by filtration and dried to give the titled compound (570 mg, 61%). MS: m/e 190 (M+1)⁺.

### Step B: 6-bromo-1H-pyrido[2,3-b][1,4]oxazin-2 (3H)-one

To a solution of 3-amino-6-bromopyridin-2 (1H)-one (190 mg, 1 mmol) in THF (10 mL) was added 2-chloroacetyl chloride (135 mg, 1.2 mL) and DIPEA (258 mg, 2 mmol). The resulting mixture was stirred at RT overnight. The reaction mixture was concentrated in vacuo. To a solution of the resulting residue in DMF (5 ml) was added K₂CO₃ (278 mg, 2 mmol). The reaction mixture was stirred at 100° C. for 4 hours and then cooled to RT. The reaction mixture was poured into H₂O and extracted by EA. The organic layer was dried over Na₂SO₄, filtered and concentrated to dryness. The resulting residue was purified by flash column chromatography to give the titled compound (220 mg, 97%). MS: m/e 230 (M+1)⁺.

### Step C: 6-bromo-2,3-dihydro-1H-pyrido[2,3-b][1,4]oxazine

To a solution of 6-bromo-1H-pyrido[2,3-b][1,4]oxazin-2 (3H)-one (200 mg, 0.97 mmol) in THF (15 mL) was added BH₃-THF (1 M, 2 mL) at 0° C. The reaction was stirred at RT overnight. The reaction mixture was poured into H₂O and extracted by EA. The organic layer was dried over Na₂SO₄, filtered, and concentrated to dryness. The resulting residue was purified by flash column chromatography to give the titled compound (110 mg, 53%). MS: m/e 216 (M+1)⁺.

### Step D: 6-bromo-1-methyl-2,3-dihydro-1H-pyrido[2,3-b][1,4]oxazine

To a solution of 6-bromo-2,3-dihydro-1H-pyrido[2,3-b][1,4]oxazine (110 mg, 0.516 mmol) in DMF (5 mL) was added CH₃I (141 mg, 1 mmol) and K₂CO₃ (278 mg, 2 mmol). The reaction was stirred at RT overnight. The reaction mixture was poured into H₂O and extracted by EA. The organic layer was dried over Na₂SO₄, filtered, and concentrated to dryness. The resulting residue was purified by flash column chromatography to give the titled compound (80 mg, 68%). MS: m/e 230 (M+1)⁺.

### Step E: 1-methyl-2,3-dihydro-1H-pyrido[2,3-b][1,4]oxazine-6-carbaldehyde

To a stirred solution of 6-bromo-1-methyl-2,3-dihydro-1H-pyrido[2,3-b][1,4]oxazine (150 mg, 0.6 mmol) in THF (10 mL), cooled to −78° C. and under a nitrogen atmosphere was added n-BuLi (1 M in hexane, 0.6 mmol, 0.6 mL) by dropwise. After stirring for 20 minutes, a solution of DMF (79 mg, 1 mmol) in THF (2 mL) was slowly added. The reaction mixture was slowly warmed up to RT and stirred overnight. The reaction mixture was poured into saturated NH₄Cl aq. and extracted by EA (15 mL×3). The combined organic layers were washed with brine, dried over Na₂SO₄, concentrated. The resulting residue was purified by flash column chromatography to give the titled compound (40 mg, 37%). MS: M/e 179 (M+1)⁺.

### Step F: 1-(1-methyl-2,3-dihydro-1H-pyrido[2,3-b][1,4]oxazin-6-yl)ethan-1-ol

To a stirred solution of 1-methyl-2,3-dihydro-1H-pyrido[2,3-b][1,4]oxazine-6-carbaldehyde (40 mg, 0.22 mmol) in THF (10 mL), cooled to 0° C. and under a nitrogen atmosphere was added CH₃MgBr (1 M in hexane, 02 mmol, 0.2 mL) dropwise. The reaction mixture was slowly warmed up to RT and stirred overnight. The reaction mixture was poured into H₂O and extracted by EA (15 mL×3). The combined organic layer was washed with brine, dried over Na₂SO₄, concentrated. The resulting residue was purified by flash column chromatography to give the titled compound (10 mg, 24%). MS: M/e 195 (M+1)⁺.

### Step G: 2-(7-((2S,5R)-2,5-diethyl-4-(1-(1-methyl-2,3-dihydro-1H-pyrido[2,3-b][1,4]oxazin-6-yl)ethyl)piperazin-1-yl)-4-methyl-5-oxo-4,5-dihydro-2H-pyrazolo[4,3-b]pyridin-2-yl)acetonitrile

To a solution of Intermediate 5 (20 mg, 0.06 mmol) in acetonitrile (5 mL) was added 1-(1-methyl-2,3-dihydro-1H-pyrido[2,3-b][1,4]oxazin-6-yl)ethan-1-ol (10 mg, 0.05 mmol), (cyanomethyl)trimethylphosphonium (125 mg, 0.5 mmol) and DIPEA (129 mg, 1 mmol). The reaction mixture was sealed and stirred under nitrogen protection at 105° C. for 16 hours. The mixture was added H₂O and extracted with ethyl acetate. The combined organic layers were dried over Na₂SO₄, filtered and concentrated. The resulting residue was purified by flash column chromatography to give the titled compound (0.88 mg, 3%). ¹H NMR (400 MHz, CD₃OD) δ 7.97 (s, 1H), 7.05 (t, J=10.2 Hz, 2H), 5.60 (s, 1H), 5.49 (s, 2H), 4.61 (s, 1H), 4.45 (d, J=4.1 Hz, 2H), 3.63 (s, 2H), 3.46 (d, J=12.2 Hz, 4H), 3.36 (s, 1H), 2.92 (s, 3H), 2.69 (s, 1H), 2.19 (t, J=7.6 Hz, 1H), 2.08-1.94 (m, 2H), 1.78 (s, 2H), 1.58 (d, J=14.2 Hz, 2H), 1.30 (s, 6H), 1.08 (s, 3H) ppm. MS: M/e 505 (M+1)⁺.

### Compound A168: 2-(7-((2S,5R)-2,5-dimethyl-4-(1-(quinoxalin-6-yl)ethyl)piperazin-1-yl)-6-fluoro-4-methyl-5-oxo-4,5-dihydro-2H-pyrazolo[4,3-b]pyridin-2-yl)acetonitrile

A mixture of 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile (40 mg, 0.09 mmol) in CH₃CN (5 mL) was heated to 80° C. in a sealed tube. Selectfluor (47 mg, 0.13 mmol) was added. The resulting mixture was stirred at 80° C. for another 30 minutes. The reaction mixture was cooled to RT, diluted with EA (20 mL) and washed with H₂O (10 mL×2). dried over Na₂SO₄, filtered and concentrated. The resulting residue was purified by pre-TLC (DCM:MeOH=15:1) to give the titled compound (10 mg, 23%). ¹H NMR (400 MHz, CD₃OD) δ 8.89-8.87 (m, 2H), 8.15-8.00 (m, 3H), 7.96 (s, 1H), 5.46 (s, 2H), 4.77-4.76 (m, 1H), 4.08-4.06 (m, 1H), 3.81-3.78 (m, 1H), 3.67-3.65 (m, 1H), 3.49 (s, 3H), 3.18-3.15 (m, 1H), 2.82-2.80 (m, 2H), 1.49-1.45 (m, 6H), 1.09 (d, J=6.4 Hz, 3H) ppm. MS: M/e 475 (M+1)⁺.

### Compound A169: 2-(cyanomethyl)-7-((2S,5R)-3-ethyl-4-(1-(quinoxalin-6-yl)ethyl)piperazin-1-yl)-4-methyl-5-oxo-4,5-dihydro-2H-pyrazolo[4,3-b]pyridine-6-carbonitrile

A mixture of 2-(6-bromo-7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile (50 mg, 0.09 mmol), Pd(PPh₃)₄ (23 mg, 0.02 mmol) and Zn(CN)₂ (21 mg, 0.18 mmol) in DMF (2 mL) was heated to 100° C. overnight under N₂ atmosphere. The reaction mixture was diluted with EA (10 mL) and washed with brine (5 mL×2). The organic layers were dried over Na₂SO₄, filtered and concentrated. The resulting residue was purified by pre-TLC (DCM: MeOH=15:1) to give the titled compound (2 mg, 4%). ¹H NMR (400 MHz, CD₃OD) δ 8.88-8.86 (m, 2H), 8.09-7.95 (m, 4H), 5.51 (s, 2H), 4.36-4.26 (m, 3H), 4.07-4.04 (m, 2H), 3.42 (s, 3H), 3.15-3.12 (m, 1H), 2.76-2.63 (m, 2H), 1.69-1.66 (m, 1H), 1.55-1.54 (m, 1H), 1.48-1.46 (m, 3H), 1.04-1.02 (m, 2.5H), 0.83-0.80 (m, 0.5H) ppm. MS: M/e 482 (M+1)⁺.

### Compound A170: 2-(7-((2S,5R)-2,5-diethyl-4-(1-(4-fluoro-2-(trifluoromethoxy)phenyl)ethyl)piperazin-1-yl)-4-methyl-5-oxo-4,5-dihydro-2H-pyrazolo[4,3-b]pyridin-2-yl)acetonitrile

### Step A: 1-(4-fluoro-2-(trifluoromethoxy)phenyl)ethan-1-ol

To a solution of 4-fluoro-2-(trifluoromethoxy)benzaldehyde (208 mg, 1 mmol) in THF at 0-5° C. under N₂ atmosphere, methylmagnesium bromide (1 M, 1.2 mL) was slowly added. After addition, the mixture was stirred at RT overnight. The reaction mixture was quenched with H₂O and extracted with EA. The combined organic layers were dried over Na₂SO₄, filtered and concentrated. The resulting residue was purified by flash column chromatography to give the titled compound (100 mg, 45%). MS: m/e 225 (M+1)⁺.

### Step B: 7-((2S,5R)-2,5-diethyl-4-(1-(4-fluoro-2-(trifluoromethoxy)phenyl)ethyl)piperazin-1-yl)-4-methyl-2-(tetrahydro-2H-pyran-2-yl)-2,4-dihydro-5H-pyrazolo[4,3-b]pyridin-5-one

To a solution of Intermediate 3 (50 mg, 0.13 mmol) in acetonitrile (5 mL) was added 4-fluoro-2-(trifluoromethoxy)benzaldehyde (50 mg, 0.22 mmol), (cyanomethyl)trimethylphosphonium (125 mg, 0.5 mmol) and DIPEA (129 mg, 1 mmol). The resulting mixture was sealed and stirred at 100° C. for 24 hours. The mixture was quenched with H₂O and extracted with EA. The organic layer was washed with brine, dried over Na₂SO₄, filtered, and concentrated to dryness. The resulting residue was purified by flash column chromatography to give the titled compound (30 mg, 40%). MS: M/e 580 (M+1)⁺.

### Step C: 7-((2S,5R)-2,5-diethyl-4-(1-(4-fluoro-2-(trifluoromethoxy)phenyl)ethyl)piperazin-1-yl)-4-methyl-2,4-dihydro-5H-pyrazolo[4,3-b]pyridin-5-one

To a solution of 7-((2S,5R)-2,5-diethyl-4-(1-(4-fluoro-2-(trifluoromethoxy)phenyl)ethyl)piperazin-1-yl)-4-methyl-2-(tetrahydro-2H-pyran-2-yl)-2,4-dihydro-5H-pyrazolo[4,3-b]pyridin-5-one (30 mg, 0.05 mmol) in DCM (10 mL) was added TFA (5 mL). The resulting mixture was stirred at RT overnight. The reaction mixture was quenched with a solution of saturated NaHCO₃ aq. and extracted by DCM. The organic layer was dried over Na₂SO₄, filtered, and concentrated to dryness. The resulting residue was purified by flash column chromatography to give the titled compound (10 mg, 40%). MS: M/e 496 (M+1)⁺.

### Step D: 2-(7-((2S,5R)-2,5-diethyl-4-(1-(4-fluoro-2-(trifluoromethoxy)phenyl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-diethyl-4-(1-(4-fluoro-2-(trifluoromethoxy)phenyl)ethyl)piperazin-1-yl)-4-methyl-2,4-dihydro-5H-pyrazolo[4,3-b]pyridin-5-one (10 mg, 0.02 mmol) in DMF (5 mL) was added 2-iodoacetonitrile (16 mg, 0.1 mmol) and K₂CO₃ (27.8 mg, 0.2 mmol). The resulting mixture was stirred at room temperature overnight. The reaction mixture was quenched with H₂O and extracted with EA. The organic layer was washed with brine, dried over Na₂SO₄, filtered, and concentrated to dryness. The resulting residue was purified by flash column chromatography (DCM/MeOH) to give the titled compound (2 mg, 11%). ¹H NMR (400 MHz, CD₃OD) δ 7.92 (s, 1H), 7.44-7.35 (m, 1H), 7.20-7.09 (m, 2H), 5.54 (s, 1H), 5.47 (s, 2H), 4.31 (s, 0.5H), 4.20 (s, 0.5H), 3.43 (m, 5H), 3.12 (s, 1H), 2.96 (s, 1H), 2.90 (s, 1H), 2.79 (s, 1H), 2.04 (s, 2H), 1.66 (s, 2H), 1.29 (s, 2H), 1.02 (t, J=8.0 Hz, 2H), 0.92 (d, J=19.8 Hz, 2H), 0.74 (t, J=7.4 Hz, 1.5H), 0.55 (s, 1.5H) ppm. MS: M/e 535 (M+1)⁺.

### Compound A171: 2-(7-((2S,5R)-4-(1-(2-(difluoromethoxy)-4-fluorophenyl)ethyl)-2,5-diethylpiperazin-1-yl)-4-methyl-5-oxo-4,5-dihydro-2H-pyrazolo[4,3-b]pyridin-2-yl)acetonitrile

### Step A: 1-(2-(difluoromethoxy)-4-fluorophenyl)ethan-1-one

A mixture of 1-bromo-2-(difluoromethoxy)-4-fluorobenzene (2.41 g, 10.0 mmol), tributyl(1-ethoxyvinyl)stannane (7.22 g, 20.0 mmol) and Pd(PPh₃)₂Cl₂ (350 mg, 0.50 mmol) in toluene (20 mL) was stirred at 100° C. under N₂ for 16 hours. The mixture was cooled and HCl (10 mL, 4M in Dioxane) was added and the resulting mixture was stirred for 10 minutes. The resulting mixture was diluted with EA (30 mL), washed with brine (20 mL×3), dried and concentrated to dryness. The resulting residue was purified by flash column chromatography to give the titled compound (2.02 g, 99%). MS: M/e 205 (M+1)⁺.

### Step B: 1-(2-(difluoromethoxy)-4-fluorophenyl)ethan-1-ol

To a solution of 1-(2-(difluoromethoxy)-4-fluorophenyl)ethan-1-one (2.02 g, 9.9 mmol) in MeOH (30 mL) was added NaBH₄ (570 mg, 15 mmol) at room temperature and the mixture was stirred at room temperature for 5 hours. The reaction mixture was quenched with saturated NaHCO₃ (15 mL) aq. and extracted with EA (15 mL×3). The combined organic layers were washed with brine (15 mL×2), dried over Na₂SO₄ and concentrated to dryness. The resulting residue was purified by flash column chromatography to give the titled compound (1.8 g, 90%). ¹H NMR (400 MHz, DMSO-d6) δ 7.67-7.52 (m, 1H), 7.50-6.99 (m, 3H), 5.28 (d, J=3.6 Hz, 1H), 5.01-4.88 (m, 1H), 1.28 (d, J=6.4 Hz, 3H) ppm. MS: M/e 189 (M−17)⁺.

### Step C: 2-(7-((2S,5R)-4-(1-(2-(difluoromethoxy)-4-fluorophenyl)ethyl)-2,5-diethylpiperazin-1-yl)-4-methyl-5-oxo-4,5-dihydro-2H-pyrazolo[4,3-b]pyridin-2-yl)acetonitrile

A mixture of Intermediate 5 (33 mg, 0.1 mmol), 1-(2-(difluoromethoxy)-4-fluorophenyl)ethan-1-ol (52 mg, 0.25 mmol), (cyanomethyl)trimethylphosphonium iodide (85 mg, 0.35 mmol) and DIPEA (80 mg, 0.62 mmol) in MeCN (1 mL) was stirred at 100° C. for 16 hours. The resulting mixture was diluted with EA (10 mL), washed with brine (5 mL×3), dried over Na₂SO₄ and concentrated to dryness. The resulting residue was purified by Prep-TLC (DCM:MeOH=20:1) to give the mixture of diastereoisomers Compound A171, which was further separated into Compound A171a (5 mg) and Compound 171b (6 mg) by Prep-HPLC (Method B).

Compound A171a (the earlier peak): ¹H NMR (400 MHz, CD₃OD) δ 7.92 (s, 1H), 7.64 (dd, J=8.8, 6.8 Hz, 1H), 7.07-7.00 (m, 1H), 6.96 (dd, J=10.0, 2.4 Hz, 1H), 6.91 (t, J=74.0, 1H), 5.55 (s, 1H), 5.46 (s, 2H), 4.22 (q, J=6.4 Hz, 1H), 3.43 (s, 3H), 3.32-3.31 (m, 1H), 3.29-3.23 (m, 1H), 2.98 (d, J=10.4 Hz, 1H), 2.88 (dd, J=12.0, 4.0 Hz, 1H), 2.42-2.33 (m, 1H), 2.14-2.02 (m, 1H), 1.87-1.74 (m, 1H), 1.60-1.46 (m, 2H), 1.38-1.24 (m, 4H), 0.94 (t, J=7.2 Hz, 3H), 0.74 (t, J=7.2 Hz, 3H) ppm. MS: M/e 517 (M+1)⁺.

Compound 171b (the later peak): ¹H NMR (400 MHz, CD₃OD) δ 7.92 (s, 1H), 7.73 (dd, J=8.8, 6.8 Hz, 1H), 7.01 (td, J=8.4, 2.4 Hz, 1H), 6.95 (dd, J=10.0, 2.0 Hz, 1H), 6.90 (t, J=73.6 Hz, 1H), 5.55 (s, 1H), 5.47 (s, 2H), 4.07 (q, J=6.4 Hz, 1H), 3.48 (d, J=12.8 Hz, 1H), 3.43 (s, 3H), 3.31 (s, 1H), 3.12 (d, J=9.6 Hz, 1H), 2.66 (dd, J=12.0, 3.2 Hz, 1H), 2.30 (d, J=12.0 Hz, 1H), 2.05-1.87 (m, 1H), 1.74-1.45 (m, 3H), 1.35-1.27 (m, 1H), 1.26 (d, J=6.8 Hz 3H), 1.02 (t, J=7.2 Hz, 3H), 0.65 (t, J=7.2 Hz, 3H) ppm. MS: M/e 517 (M+1)⁺.

### Compound A172: 2-(7-((2S,5R)-4-(1-(2-(difluoromethyl)-4-fluorophenyl)ethyl)-2,5-diethylpiperazin-1-yl)-4-methyl-5-oxo-4,5-dihydro-2H-pyrazolo[4,3-b]pyridin-2-yl)acetonitrile

### Step A: 1-(2-(difluoromethyl)-4-fluorophenyl)ethan-1-ol

To a solution of 1-bromo-2-(difluoromethyl)-4-fluorobenzene (200 mg, 0.8889 mmol) in THF (20 mL) was added nBuLi (0.8 mL, 1.333 mmol) dropwise at −78° C. and stirred for 1 hour. Then acetaldehyde (78 mg, 1.778 mmol) was added dropwise at −78° C. and stirred for another 1 hour. The reaction mixture was quenched with water and extracted with DCM (100 mL). The organic layer was washed with water, dried over Na₂SO₄ and concentrated to dryness. The resulting residue was purified by flash column chromatography (PE:EA=1:1) to give the titled compound (120 mg, 71%). ¹H NMR (400 MHz, DMSO) δ 7.79-7.49 (m, 1H), 7.49-7.41 (m, 2H), 7.34 (t, J=9.2 Hz, 1H), 5.74 (s, 1H), 5.25 (q, J=6.6 Hz, 1H), 1.41 (d, J=6.7 Hz, 3H). M/e 191 (M+1)⁺.

### Step B: 2-(7-((2S,5R)-4-(1-(2-(difluoromethyl)-4-fluorophenyl)ethyl)-2,5-diethylpiperazin-1-yl)-4-methyl-5-oxo-4,5-dihydro-2H-pyrazolo[4,3-b]pyridin-2-yl)acetonitrile

A mixture of Intermediate 5 (50 mg, 0.1524 mmol), 1-(2-(difluoromethyl)-4-fluorophenyl)ethan-1-ol (58 mg, 0.3049 mmol), (cyanomethyl)trimethylphosphonium iodide (74 mg, 0.3049 mmol), DIPEA (98 mg, 0.7622 mmol) in CH₃CN (5 mL) was stirred under N₂ at 105° C. overnight. The reaction mixture was quenched with H₂O and extracted with DCM. The organic layer was washed with brine, dried over Na₂SO₄ and concentrated to dryness. The resulting residue was purified by Prep-TLC (DCM:MeOH=20:1) to give a mixture of diastereoisomers Compound A172, which was further separated into Compound A172a (15 mg, 20%) and Compound A172b (10 mg, 13%) by Prep-HPLC (Method A).

Compound A172a (the earlier peak): ¹H NMR (400 MHz, CD₃OD) δ 7.93 (s, 1H), 7.78 (s, 1H), 7.26 (dd, J=19.9, 9.0 Hz, 2H), 5.55 (s, 1H), 5.47 (s, 2H), 3.98 (d, J=5.6 Hz, 1H), 3.49 (d, J=12.3 Hz, 1H), 3.43 (s, 3H), 3.18 (d, J=10.8 Hz, 3H), 2.89-2.73 (m, 1H), 2.69 (d, J=12.5 Hz, 1H), 2.25 (d, J=12.9 Hz, 1H), 1.96-1.85 (m, 1H), 1.71-1.53 (m, 3H), 1.32 (d, J=6.5 Hz, 3H), 1.03 (t, J=7.3 Hz, 3H), 0.59 (t, J=7.4 Hz, 3H) ppm. MS: M/e 501 (M+1)⁺

Compound A172b (the later peak): ¹H NMR (400 MHz, CD₃OD) δ 7.92 (s, 1H), 7.70-7.59 (m, 1H), 7.34 (d, J=9.2 Hz, 1H), 7.25 (t, J=8.0 Hz, 1H), 5.57 (s, 1H), 5.46 (s, 2H), 4.09 (d, J=6.8 Hz, 1H), 3.43 (s, 3H), 3.29-3.26 (m, 1H), 3.08 (d, J=12.4 Hz, 1H), 3.00-2.55 (m, 3H), 2.37 (s, 1H), 2.07 (s, 1H), 1.85 (dd, J=14.0, 6.7 Hz, 1H), 1.79-1.38 (m, 3H), 1.36 (d, J=6.5 Hz, 3H), 1.03-0.88 (m, 3H), 0.68 (t, J=7.1 Hz, 3H) ppm. MS: M/e 501 (M+1)⁺

### Compound A177: 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridin-2-yl)acetonitrile

### Step A: 4-(3,4-dimethoxybenzyl)-7-((2S,5R)-5-ethyl-2-methyl-4-(1-(quinoxalin-6-yl)ethyl)piperazin-1-yl)-2-(tetrahydro-2H-pyran-2-yl)-2,4-dihydro-5H-pyrazolo[4,3-b]pyridin-5-one

To a solution of 6-(1-((2R,5S)-2-ethyl-5-methylpiperazin-1-yl)ethyl)quinoxaline (200 mg, 0.702 mmol) and Intermediate 8 (399 mg, 0.772 mmol) in CH₃CN (2 mL) at room temperature was added DIPEA (181 mg, 1.404 mmol). The mixture was stirred at 105° C. for 24 hours. Then the mixture was concentrated under reduced pressure. The resulting residue was purified by flash column chromatography to give the titled compound (200 mg, 44%). MS: M/e 652 (M+1)⁺.

### Step B: 7-((2S,5R)-5-ethyl-2-methyl-4-(1-(quinoxalin-6-yl)ethyl)piperazin-1-yl)-2,4-dihydro-5H-pyrazolo[4,3-b]pyridin-5-one

To a solution of 4-(3,4-dimethoxyphenyl)-7-((2S,5R)-5-ethyl-2-methyl-4-(1-(quinoxalin-6-yl)ethyl)piperazin-1-yl)-2-(tetrahydro-2H-pyran-2-yl)-2,4-dihydro-5H-pyrazolo[4,3-b]pyridin-5-one (200 mg, 0.307 mmol) in TFA (3 mL) and TfOH (3 mL) was stirred at 60° C. for 12 hours. The reaction mixture was concentrated under reduced pressure. The resulting residue was dissolved in water. The aqueous was basified by Na₂CO₃ (4M) to pH ˜10 and extracted with DCM (35 mL×3). The combined organic layer was washed with brine (20 mL×3), dried over Na₂SO₄ and concentrated under reduced pressure. The resulting residue was purified by flash column chromatography to give the titled compound (100 mg, 78%). MS: M/e 418 (M+1)⁺.

### Step C: 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridin-2-yl)acetonitrile

To a solution of 7-((2S,5R)-5-ethyl-2-methyl-4-(1-(quinoxalin-6-yl)ethyl)piperazin-1-yl)-2,4-dihydro-5H-pyrazolo[4,3-b]pyridin-5-one (100 mg, 0.240 mmol) and K₂CO₃ (67 mg, 0.464 mmol) in DMF (5 mL) at room temperature was added 2-iodoacetonitrile (44 mg, 0.264 mmol). The mixture solution was stirred at room temperature for 12 hours. Then the reaction mixture was quenched with saturated NaCl (20 mL) at room temperature and extracted with EA (30 mL×2). The combined organic layer was dried over Na₂SO₄ and concentrated under reduced pressure, to obtain Compound A177. The resulting residue containing Compound A177 was purified to give the titled Compound A177a (23 mg) and Compound A177b (24 mg) by Prep-HPLC (Method A).

Compound A177a (the earlier peak): ¹H NMR (400 MHz, CDCl₃) δ 11.51 (s, 1H), 8.85 (s, 2H), 8.09-8.13 (m, 1H), 8.03 (s, 1H), 7.93-7.91 (d, J=8 Hz, 1H), 7.61-7.57 (m, 1H), 5.57 (s, 1H), 5.08 (s, 2H), 4.68-4.64 (m, 2H), 4.03-3.98 (m, 1H), 3.30-3.27 (m, 1H), 3.01-2.99 (m, 1H), 2.86-2.83 (m, 1H), 2.45-2.43 (d, J=8 Hz, 1H), 1.59-1.57 (m, 2H), 1.43-1.25 (m, 6H), 0.70 (t, J=4 Hz, 3H) ppm. MS: M/e 457 (M+1)⁺.

Compound A177b (the later peak): ¹H NMR (400 MHz, CDCl₃) δ 11.68 (s, 1H), 8.84 (s, 2H), 8.11-8.08 (m, 2H), 8.05 (s, 1H), 7.99-7.96 (m, 1H), 5.57 (s, 1H), 5.11 (s, 2H), 4.86-4.40 (m, 2H), 3.88-3.84 (m, 1H), 3.58-3.54 (m, 1H), 3.20-3.16 (m, 1H), 2.82-2.77 (m, 1H), 2.24-2.20 (m, 1H), 1.79-1.76 (m, 1H), 1.60-1.56 (m, 1H), 1.46-1.42 (m, 3H), 1.25-1.21 (m, 3H), 1.05 (t, J=8 Hz, 3H) ppm. MS: M/e 457 (M+1)⁺.

### Compound A179: 2-(6-bromo-7-((2S,5R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 2-(7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile (170 mg, 0.37 mmol) in CH₃CN (5 mL) were added NBS (65 mg, 0.37 mmol) slowly at room temperature. The resulting mixture was stirred at room temperature for another 2 hours. Then the reaction mixture was quenched with saturated H₂O (20 mL) and extracted with EA (30 mL×2). The organic layers were dried over Na₂SO₄, filtered and concentrated. The resulting residue was purified by flash column chromatography (DCM:MeOH=15:1) to give Compound A179 (120 mg, 61%) as a mixture. The Compound A179 (20 mg, mixture) was further purified by Prep-TLC to give Compound A179a (2 mg) and Compound A179b (2 mg).

Compound A179a: ¹H NMR (400 MHz, CD₃OD) δ 8.88-8.87 (m, 2H), 8.12-7.99 (m, 3H), 7.94 (s, 1H), 5.47 (s, 2H), 4.38 (br s, 1H), 4.01-3.98 (m, 1H), 3.83-3.78 (m, 1H), 3.68-3.64 (m, 2H), 3.51 (s, 3H), 2.88-2.85 (m, 1H), 2.56-2.54 (m, 1H), 1.56-1.54 (m, 3H), 0.90-0.88 (m, 3H), 0.72-0.68 (m, 3H) ppm. MS: M/e 535 (M+1)⁺.

Compound A179b: ¹H NMR (400 MHz, CD₃OD) δ 8.88-8.86 (m, 2H), 8.11-8.10 (m, 3H), 7.97 (s, 1H), 5.49 (s, 2H), 4.38 (br s, 1H), 4.01-4.00 (m, 1H), 3.73-3.71 (m, 2H), 3.48 (s, 3H), 2.76-2.70 (m, 3H), 2.52-2.50 (m, 1H), 1.83-1.80 (m, 2H), 1.50-1.47 (m, 3H), 1.01-0.98 (m, 3H) ppm. MS: M/e 535 (M+1)⁺.

### Compound A180: 2-(7-((2S,5R)-4-(1-(6-(difluoromethoxy)pyridin-3-yl)ethyl)-2,5-diethylpiperazin-1-yl)-4-methyl-5-oxo-4,5-dihydro-2H-pyrazolo[4,3-b]pyridin-2-yl)acetonitrile

### Step A: 5-bromo-2-(difluoromethoxy)pyridine

A mixture of 5-bromopyridin-2-ol (1 g, 5.75 mmol), ClCF₂COONa (0.876 g, 5.75 mmol), Cs₂CO₃ (2.81 g, 8.62 mmol) in DMF (20 mL) was heated at 100° C. for 3 hours. The reaction was quenched with water and extracted with EA. The organic layer was separated, washed with water (20 mL), brine (20 mL), dried over Na₂SO₄, filtered and concentrated. The resulting residue was purified by flash column chromatography (PE:EA=100:1) to give the titled compound (220 mg, 17%). MS: M/e 224 (M+1)⁺.

### Step B: 1-(6-(difluoromethoxy)pyridin-3-yl)ethan-1-one

A mixture of 5-bromo-2-(difluoromethoxy)pyridine (220 mg, 0.99 mmol), tributyl(1-ethoxyvinyl)stannane (543 mg, 1.5 mmol) and Pd(PPh₃)₄ (120 mg, 0.01 mmol) in toluene (5 mL) was heated to 10° C. for overnight under N₂ atmosphere. The solvent was removed under vacuum. The resulting residue was dissolved in HCl dioxane solution (5 mL, 4M). The reaction mixture was stirred at room temperature for 10 minutes and concentrated. The resulting residue purified by Prep-TLC (PE:EA=20:1) to give the titled compound (90 mg, 49%). MS: M/e 188 (M+1)⁺.

### Step C: 1-(6-(difluoromethoxy)pyridin-3-yl)ethan-1-ol

To a solution of 1-(6-(difluoromethoxy)pyridin-3-yl)ethan-1-one (90 mg, 0.49 mmol) in MeOH (5 mL) was added NaBH₄ (20 mg, 0.5 mmol) at 0° C. The reaction was stirred at room temperature for 30 min. The reaction was quenched by H₂O (2 mL). The solvent was removed under vacuum. The resulting residue was purified by flash column chromatography (PE:EA=10:1) to give the titled compound (60 mg, 64%). MS: M/e 190 (M+1)⁺.

### Step D: 2-(7-((2S,5R)-4-(1-(6-(difluoromethoxy)pyridin-3-yl)ethyl)-2,5-diethylpiperazin-1-yl)-4-methyl-5-oxo-4,5-dihydro-2H-pyrazolo[4,3-b]pyridin-2-yl)acetonitrile

A mixture of Intermediate 5 (35 mg, 0.1 mmol), 3-(1-hydroxyethyl)pyridin-2 (1H)-one (40 mg, 0.2 mmol), (cyanomethyl)trimethylphosphonium iodide (52 mg, 0.2 mmol) and DIPEA (130 mg, 1 mmol) in CH₃CN (2 mL) was heated to 105° C. overnight under N₂ atmosphere. The reaction solvent was removed under vacuum. The resulting residue was purified by pre-TLC (DCM:MeOH=15:1) to give the crude product Compound A180. The Compound A180 was further purified by Prep-HPLC (Method A) to give two compounds Compound A180a (2 mg) and Compound A180b (2 mg).

Compound A180a (the earlier peak): ¹H NMR (400 MHz, CD₃OD) δ 7.92 (s, 1H), 7.76-7.74 (m, 2H), 7.62 (s, 1H), 6.61 (d, J=9.3 Hz, 1H), 5.57 (s, 1H), 5.47 (s, 2H), 3.69-3.67 (m, 1H), 3.43 (s, 3H), 3.31-3.30 (m, 1H), 2.93-2.83 (m, 2H), 2.75-2.55 (m, 2H), 2.49-2.46 (m, 1H), 2.08-2.06 (m, 1H), 1.82-1.80 (m, 1H), 1.57-1.54 (m, 2H), 1.33 (d, J=6.4 Hz, 3H), 0.93 (t, J=7.5 Hz, 3H), 0.81 (t, J=7.4 Hz, 3H) ppm. MS: M/e 500 (M+1)⁺.

Compound A180b (the later peak): ¹H NMR (400 MHz, CD₃OD) δ 7.93 (s, 1H), 7.76 (s, 1H), 7.76 (t, J=60.0 Hz, 1H), 7.65-7.64 (m, 1H), 6.58 (d, J=9.6 Hz, 1H), 5.56 (s, 1H), 5.47 (s, 2H), 3.52-3.44 (m, 3H), 3.43 (s, 3H), 3.10-3.07 (m, 1H), 2.79-2.61 (m, 2H), 2.49-2.46 (m, 1H), 1.90-1.87 (m, 1H), 1.69-1.65 (m, 2H), 1.52-1.50 (m, 1H), 1.31 (d, J=6.6 Hz, 3H), 1.00 (t, J=7.3 Hz, 3H), 0.73 (t, J=7.4 Hz, 3H) ppm. MS: M/e 500 (M+1)⁺.

Compound A181: Mixture of 2-(7-((2S,5R)-2,5-diethyl-4-(1-(1-ethyl-5-methyl-1H-imidazol-2-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-diethyl-4-(1-(1-ethyl-4-methyl-1H-imidazol-2-yl)ethyl)piperazin-1-yl)-4-methyl-5-oxo-4,5-dihydro-2H-pyrazolo[4,3-b]pyridin-2-yl)acetonitrile

### Step A: mixture of 1-ethyl-4-methyl-1H-imidazole-2-carbaldehyde and 1-ethyl-5-methyl-1H-imidazole-2-carbaldehyde

A sealed tube was charged with 4-methyl-1H-imidazole-2-carbaldehyde (220 mg, 2 mmol), iodoethane (343 mg, 2.2 mmol), potassium carbonate (552 mg, 4 mmol) and DMF (5 ml). The mixture was stirred at 60° C. overnight, cooled to RT.Water was added and the aqueous was extracted with EA. The combined organic layers were washed with brine, dried over Na₂SO₄, and concentrated to give the titled product (250 mg, crude) which was used directly to the next step without further purification. MS: M/e 139 (M+1)⁺.

### Step B: mixture of 1-(1-ethyl-4-methyl-1H-imidazol-2-yl)ethan-1-ol and 1-(1-ethyl-5-methyl-1H-imidazol-2-yl)ethan-1-ol

To a solution of [1-ethyl-4-methyl-1H-imidazole-2-carbaldehyde and 1-ethyl-5-methyl-1H-imidazole-2-carbaldehyde](250 mg, 1.8 mmol) in THF (5 mL) at 0° C. was added MeMgBr (3.0 M in Et₂O, 1 mL, 2.7 mmol) and the mixture stirred for 1 hour, saturated NH₄Cl aq. (40 mL) and EA (20 mL) were added. The aqueous phase was extracted with EA (2×20 mL). The organic layer was washed with brine (20 mL), dried over Na₂SO₄ and concentrated under reduced pressure to give the titled product (1:1, the ratio was confirmed by H-NMR, 160 mg) as a mixture. ¹H NMR (400 MHz, DMSO-d6) δ 6.77 (s, 1H), 6.49 (s, 1H), 5.17 (s, 2H), 4.80-4.67 (m, 2H), 3.93 (dtd, J=21.8, 14.7, 7.3 Hz, 4H), 2.15 (s, 3H), 2.02 (s, 3H), 1.42 (t, J=6.8 Hz, 6H), 1.27-1.18 (m, 6H) ppm. MS: M/e 155 (M+1)⁺.

### Step C: mixture of 2-(7-((2S,5R)-2,5-diethyl-4-(1-(1-ethyl-5-methyl-1H-imidazol-2-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-diethyl-4-(1-(1-ethyl-4-methyl-1H-imidazol-2-yl)ethyl)piperazin-1-yl)-4-methyl-5-oxo-4,5-dihydro-2H-pyrazolo[4,3-b]pyridin-2-yl)acetonitrile

To a solution of [1-(1-ethyl-4-methyl-1H-imidazol-2-yl)ethan-1-ol and 1-(1-ethyl-5-methyl-1H-imidazol-2-yl)ethan-1-ol](34 mg, 0.22 mmol) in MeCN (3 mL) was added Intermediate 5 (60 mg, 0.18 mmol), (cyanomethyl)trimethylphosphonium iodide (87 mg, 0.36 mmol) and DIPEA (93 mg, 0.72 mmol). The reaction mixture was stirred at 105° C. overnight. The reaction mixture was quenched with water, extracted with EA. The combined organic layers were washed with brine, dried over Na₂SO₄, filtered, and concentrated to dryness. The resulting residue was purified by Prep-HPLC to give the titled product (2 mg, 3%) as a mixture. ¹H NMR (400 MHz, CD₃OD) δ 8.37 (s, 1H, HCOOH), 7.94 (d, J=3.3 Hz, 1H), 7.20-6.97 (m, 1H), 5.56 (d, J=5.3 Hz, 1H), 5.47 (d, J=2.9 Hz, 2H), 4.44-4.13 (m, 4H), 3.56-3.37 (m, 4H), 2.93-2.87 (m, 2H), 2.50-2.37 (m, 0.5H), 2.33-2.29 (m, 3H), 2.18 (d, J=12.3 Hz, 0.5H), 1.97-1.60 (m, 3H), 1.57-1.33 (m, 8H), 1.02 (t, J=7.3 Hz, 1.5H), 0.87-0.83 (m, 3H), 0.73 (t, J=7.3 Hz, 1.5H) ppm. MS: M/e 465 (M+1)⁺.

### Compound A182: 2-(7-((2S,5R)-2,5-diethyl-4-(1-(1-ethyl-4,5,6,7-tetrahydro-1H-benzo[d]imidazol-2-yl)ethyl)piperazin-1-yl)-4-methyl-5-oxo-4,5-dihydro-2H-pyrazolo[4,3-b]pyridin-2-yl)acetonitrile

### Step A: 1-ethyl-4,5,6,7-tetrahydro-1H-benzo[d]imidazole

To a solution of 4,5,6,7-tetrahydro-1H-benzo[d]imidazole (1.8 g, 14.8 mmol) in DMSO (30 mL), KOH (1.46 g, 22.1 mmol, 85%) was added. The reaction mixture was stirred at RT overnight. Then bromoethane (2 g, 18.4 mmol) was added. The resulting mixture was stirred for another 6 hours at RT. The reaction mixture was poured into ice-water (250 mL) and treated with 5N NaOH (50 mL), then extracted with DCM (200 mL). The organic layer was washed with water (100 mL), dried over Na₂SO₄, filtered, and concentrated to dryness. The resulting residue was purified by flash column chromatography (DCM/MeOH) to give the titled compound (2 g, 90%). ¹H NMR (400 MHz, CDCl3) δ 7.35 (s, 1H), 3.83 (q, J=7.3 Hz, 2H), 3.48 (s, 2H), 2.64-2.46 (m, 2H), 1.92-1.72 (m, 4H), 1.38 (t, J=7.3 Hz, 3H) ppm.

### Step B: 1-(1-ethyl-4,5,6,7-tetrahydro-1H-benzo[d]imidazol-2-yl)ethan-1-ol

To a solution of 1-ethyl-4,5,6,7-tetrahydro-1H-benzo[d]imidazole (500 mg, 3.3 mmol) in THF (15 mL) were added n-BuLi (2.9 mL, 1.6M, 4.6 mmol) at −78° C. The reaction mixture was stirred at −78° C. for 1 hour, acetaldehyde (567 mg, 16.6 mmol) was added and stirred at below −70° C. for another 1 hour. The reaction mixture was quenched by saturated NH₄Cl aq. and extracted by EA. The organic layer was washed with water, dried over Na₂SO₄, filtered, and concentrated to dryness. The resulting residue was purified by flash column chromatography (DCM/MeOH) to give the titled compound (510 mg). MS: M/e 195 (M+1)⁺.

### Step C: 2-(7-((2S,5R)-2,5-diethyl-4-(1-(1-ethyl-4,5,6,7-tetrahydro-1H-benzo[d]imidazol-2-yl)ethyl)piperazin-1-yl)-4-methyl-5-oxo-4,5-dihydro-2H-pyrazolo[4,3-b]pyridin-2-yl)acetonitrile

To a solution of Intermediate 5 (30 mg, 0.091 mmol) in CH₃CN (3 mL) was added 1-(1-ethyl-4,5,6,7-tetrahydro-1H-benzo[d]imidazol-2-yl)ethan-1-ol (35 mg, 0.183 mmol), (cyanomethyl)trimethylphosphonium iodide (89 mg, 0.37 mmol) and DIPEA (118 mg, 0.91 mmol). The reaction mixture was stirred at 105° C. for 24 hours. The reaction solvent was removed under reduce pressure. The resulting residue was purified by Prep-HPLC (Method B) to give the titled compound (1 mg, 3%). ¹H NMR (400 MHz, CD₃OD) δ 7.92 (s, 1H), 5.59-5.45 (m, 3H), 4.32-3.80 (m, 4H), 3.54-3.31 (m, 5H), 2.98-2.60 (m, 4H), 2.58-2.30 (m, 5H), 1.95-1.51 (m, 6H), 1.50-1.23 (m, 6H), 1.05-0.60 (m, 6H) ppm. MS: M/e 505 (M+1)⁺.

### Compound A183: 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-d]pyrimidin-2-yl)acetonitrile

### Step A: 4-((4-methoxybenzyl)amino)-1-(tetrahydro-2H-pyran-2-yl)-1H-pyrazole-3-carboxamide

To a solution of 4-amino-1-(tetrahydro-2H-pyran-2-yl)-1H-pyrazole-3-carboxamide (0.5 g, 2.38 mmol) and 4-methoxybenzaldehyde (323 mg, 2.38 mmol) in DCM (14 mL) was added NaBH(OAc)₃ (1 g 4.76 mmol). The resulting mixture was stirred at room temperature for 16 hours. The resulting mixture was quenched with water and extracted with DCM (60 mL×2). The organic layer was washed with brine, dried over Na₂SO₄, filtered, and concentrated to dryness. The resulting residue was purified by flash column chromatography to give the titled compound (0.6 g, 76%). MS: M/e 331 (M+1)⁺.

### Step B: 4-(4-methoxybenzyl)-2-(tetrahydro-2H-pyran-2-yl)-2,4-dihydro-5H-pyrazolo[4,3-d]pyrimidine-5,7 (6H)-dione

To a solution of 4-((4-methoxybenzyl)amino)-1-(tetrahydro-2H-pyran-2-yl)-1H-pyrazole-3-carboxamide (560 mg, 1.7 mmol) in DMF (10 mL) was added NaH (136 mg, 3.4 mmol) at 0° C. After 1 hour, to the above solution, CDI (550 mg, 3.4 mmol) was added and the resulting mixture was heated to 80° C. overnight. The reaction mixture was cooled to room temperature, quenched with H₂O, concentrated to dryness. The resulting residue was purified by flash column chromatography (DCM:MeOH=10:1) to give titled compound (0.28 g, 46%). MS: M/e 357 (M+1)⁺.

### Step C: 7-((2S,5R)-5-ethyl-2-methyl-4-(1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-d]pyrimidin-5-one

To a solution of 4-(4-methoxybenzyl)-2-(tetrahydro-2H-pyran-2-yl)-2,4-dihydro-5H-pyrazolo[4,3-d]pyrimidine-5,7 (6H)-dione (100 mg, 0.28 mmol) in CH₃CN (4 mL) was added DIPEA (72 mg, 0.56 mmol), followed by POCl₃ (88 mg, 0.57 mmol) and catalyst amount DMF. The reaction was stirred at 80° C. for 6 hours in a sealed tube. The reaction mixture was cooled to RT, quenched with saturated NaHCO₃ solution and extracted with EA (60 mL×2). The organic layer was washed with brine, dried over Na₂SO₄, filtered, and concentrated to dryness. The resulting residue was dissolved in CH₃CN (3 mL), 6-(1-((2R,5S)-2-ethyl-5-methylpiperazin-1-yl)ethyl)quinoxaline (40 mg, 0.14 mmol) and DIPEA (51 mg, 0.4 mmol) were added. The resulting mixture was heated at 80° C. for 15 hours. The reaction mixture was quenched with water and extracted with EA (60 mL×3). The organic layer was washed with brine, dried over Na₂SO₄, filtered, and concentrated to dryness. The resulting residue was purified by flash column chromatography (DCM:MeOH=15:1) to give titled compound (80 mg, 45%). MS: M/e 623 (M+1)⁺.

### Step D: 7-((2S,5R)-5-ethyl-2-methyl-4-(1-(quinoxalin-6-yl)ethyl)piperazin-1-yl)-2,4-dihydro-5H-pyrazolo[4,3-d]pyrimidin-5-one

To a solution of 7-((2S,5R)-5-ethyl-2-methyl-4-(1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-d]pyrimidin-5-one (80 mg, 0.128 mmol) in TFA (2 mL) was added trifluoromethanesulfonic acid (4 mL). The resulting mixture was stirred at room temperature overnight. The reaction mixture was quenched with ice-water, basified to pH 7-8 with saturated Na₂CO₃ aq. and extracted with DCM:IPA (3:1, 60 mL×3). The combined organic layers were washed with brine, dried over Na₂SO₄, filtered and concentrated to dryness. The resulting residue was purified by Prep-TLC to give the titled compound (27 mg, 50%). MS: M/e 419 (M+1)⁺.

### Step E: 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-d]pyrimidin-2-yl)acetonitrile

To a solution of 7-((2S,5R)-5-ethyl-2-methyl-4-(1-(quinoxalin-6-yl)ethyl)piperazin-1-yl)-2,4-dihydro-5H-pyrazolo[4,3-d]pyrimidin-5-one (27 mg, 0.06 mmol) and K₂CO₃ (18 mg, 0.13 mmol) in DMF (2 mL) was added 2-iodoacetonitrile (16 mg, 0.096 mmol). The reaction was stirred at room temperature overnight. The reaction mixture was quenched with water, extracted with EA (60 mL). The organic layer was washed with brine, dried over Na₂SO₄, filtered, and concentrated to dryness. The resulting residue was purified by Prep-TLC (DCM:MeOH=15:1) to obtain Compound A183, and further purified by Prep-HPLC (Method B) to give Compound A183a (2 mg) and Compound A183b (2 mg).

Compound A183a (the earlier isomer): ¹HNMR (400 MHz, CD₃OD) δ 8.88 (d, J=3.2 Hz, 2H), 8.16-8.10 (m, 1H), 8.08-8.00 (m, 2H), 7.77-7.69 (m, 1H), 6.00-5.85 (m, 0.5H), 5.60-5.48 (m, 1.5H), 5.42 (s, 1H), 5.38-5.28 (m, 0.5H), 5.02-4.93 (m, 0.5H), 4.15-4.00 (m, 1H), 3.66-3.52 (m, 0.5H), 3.25-3.16 (m, 0.5H), 3.05-2.90 (m, 2H), 2.55-2.40 (m, 1H), 1.71-1.35 (m, 8H), 0.80-0.65 (m, 3H) ppm. MS: M/e 458 (M+1)⁺.

Compound A183b (the later isomer): ¹HNMR (400 MHz, CD₃OD) δ 8.87 (d, J=4.4 Hz, 2H), 8.14-8.02 (m, 3H), 7.77-7.71 (m, 1H), 5.89-5.80 (m, 0.5H), 5.72-5.60 (m, 0.5H), 5.56-5.40 (m, 2H), 5.31-5.22 (m, 0.5H), 5.15-5.02 (m, 0.5H), 3.96-3.80 (m, 2H), 3.52-3.42 (m, 0.5H), 3.28-3.21 (m, 0.5H), 2.91-2.65 (m, 2H), 2.34-2.24 (m, 1H), 1.70-1.50 (m, 2H), 1.45 (d, J=6.0 Hz, 3H), 1.36 (d, J=6.4 Hz, 1H), 1.29 (d, J=6.4 Hz, 2H), 1.13-1.01 (m, 3H) ppm. MS: M/e 458 (M+1)⁺.

### Compound A189: 2,2′-(6-cyano-7-((2S,5R)-5-ethyl-2-methyl-4-(1-(quinoxalin-6-yl)ethyl)piperazin-1-yl)-5-oxo-2H-pyrazolo[4,3-b]pyridine-2,4 (5H)-diyl)diacetonitrile

### Step A: 4-(3,4-dimethylbenzyl)-7-((2S,5R)-5-ethyl-2-methyl-4-(1-(quinoxalin-6-yl)ethyl)piperazin-1-yl)-2-(tetrahydro-2H-pyran-2-yl)-2,4-dihydro-5H-pyrazolo[4,3-b]pyridin-5-one

A mixture of 4-(3,4-dimethylbenzyl)-5-oxo-2-(tetrahydro-2H-pyran-2-yl)-4,5-dihydro-2H-pyrazolo[4,3-b]pyridin-7-yl trifluoromethanesulfonate (120 mg, 0.25 mmol), 6-(1-((2R,5S)-2-ethyl-5-methylpiperazin-1-yl)ethyl)quinoxaline (107 mg, 0.38 mmol) and DIPEA (170 mg, 1.3 mmol) in CH₃CN (2 mL) was heated to 105° C. for overnight under N₂ atmosphere in a sealed tube. The solvent was removed under vacuum. The resulting residue was purified by pre-TLC (DCM:MeOH=15:1) to give the titled compound (120 mg, 78%). MS: M/e 620 (M+1)⁺.

### Step B: 6-bromo-4-(3,4-dimethylbenzyl)-7-((2S,5R)-5-ethyl-2-methyl-4-(1-(quinoxalin-6-yl)ethyl)piperazin-1-yl)-2-(tetrahydro-2H-pyran-2-yl)-2,4-dihydro-5H-pyrazolo[4,3-b]pyridin-5-one

To a solution of 4-(3,4-dimethylbenzyl)-7-((2S,5R)-5-ethyl-2-methyl-4-(1-(quinoxalin-6-yl)ethyl)piperazin-1-yl)-2-(tetrahydro-2H-pyran-2-yl)-2,4-dihydro-5H-pyrazolo[4,3-b]pyridin-5-one (120 mg, 0.19 mmol) in CH₃CN (5 mL) were added NBS (34 mg, 0.19 mmol) slowly at room temperature. The resulting mixture was stirred at room temperature for another 2 hours. The reaction mixture was diluted with EA (20 mL) and washed with water (10 mL×2). The organic layers were concentrated. The resulting residue was purified by flash column chromatography (DCM:MeOH=15:1) to give the titled compound (110 mg, 82%). MS: M/e 698 (M+1)⁺.

### Step C: 6-bromo-7-((2S,5R)-5-ethyl-2-methyl-4-(1-(quinoxalin-6-yl)ethyl)piperazin-1-yl)-2,4-dihydro-5H-pyrazolo[4,3-b]pyridin-5-one

To a solution of 6-bromo-4-(3,4-dimethylbenzyl)-7-((2S,5R)-5-ethyl-2-methyl-4-(1-(quinoxalin-6-yl)ethyl)piperazin-1-yl)-2-(tetrahydro-2H-pyran-2-yl)-2,4-dihydro-5H-pyrazolo[4,3-b]pyridin-5-one (110 mg, 0.15 mmol) in TFA (2 mL) was added TfOH (5 mL) at room temperature. The resulting mixture was stirred at room temperature overnight. The reaction solvent was removed under vacuum. The crude product was dissolved with DCM (10 mL) and the aqueous was neutralized with NaOH aq. to pH=8. The organic layer was separated and concentrated. The resulting residue was purified by Prep-TLC (DCM:MeOH=10:1) to give the titled compound (60 mg, 76%). MS: M/e 496 (M+1)⁺.

### Step D: 2,2′-(6-bromo-7-((2S,5R)-5-ethyl-2-methyl-4-(1-(quinoxalin-6-yl)ethyl)piperazin-1-yl)-5-oxo-2H-pyrazolo[4,3-b]pyridine-2,4 (5H)-diyl)diacetonitrile

To a solution of 6-bromo-7-((2S,5R)-5-ethyl-2-methyl-4-(1-(quinoxalin-6-yl)ethyl)piperazin-1-yl)-2,4-dihydro-5H-pyrazolo[4,3-b]pyridin-5-one (60 mg, 0.12 mmol) and K₂CO₃ (34 mg, 0.24 mmol) in DMF (2 mL) were added 2-iodoacetonitrile (30 mg, 0.18 mmol). The resulting mixture was stirred at room temperature overnight. The reaction mixture was diluted with EA (10 mL) and washed with brine (10 mL×3). The organic layers were concentrated under reduced pressure. The resulting residue was purified by Prep-TLC (DCM:MeOH=15:1) to give the titled compound (18 mg, 26%). MS: M/e 574 (M+1)⁺.

### Step E: 2,2′-(6-cyano-7-((2S,5R)-5-ethyl-2-methyl-4-(1-(quinoxalin-6-yl)ethyl)piperazin-1-yl)-5-oxo-2H-pyrazolo[4,3-b]pyridine-2,4 (5H)-diyl)diacetonitrile

A mixture of 2,2′-(6-bromo-7-((2S,5R)-5-ethyl-2-methyl-4-(1-(quinoxalin-6-yl)ethyl)piperazin-1-yl)-5-oxo-2H-pyrazolo[4,3-b]pyridine-2,4 (5H)-diyl)diacetonitrile (18 mg, 0.03 mmol), Pd(PPh₃)₄ (12 mg, 0.01 mmol) and Zn(CN)₂ (8 mg, 0.06 mmol) in DMF (2 mL) was heated to 100° C. for overnight under N₂ atmosphere. The reaction mixture was diluted with EA (10 mL) and washed with brine (5 mL×2). The organic layers were concentrated. The resulting residue was purified by Prep-TLC (DCM:MeOH=15:1) to give the titled compound (1.56 mg, 10%). ¹H NMR (400 MHz, CD₃OD) δ 8.88-8.87 (m, 2H), 8.17-8.06 (m, 4H), 5.55 (s, 2H), 5.01 (s, 2H), 3.97-3.95 (m, 2H), 2.85-2.53 (m, 3H), 2.34-2.31 (m, 1H), 1.49-1.42 (m, 6H), 1.05-0.84 (m, 6H) ppm. MS: M/e 521 (M+1)⁺.
to give.

### Compound A194: 2-(7-((2S,5R)-5-ethyl-2-methyl-4-(1-(quinoxalin-6-yl)ethyl)piperazin-1-yl)-6-fluoro-5-oxo-4,5-dihydro-2H-pyrazolo[4,3-b]pyridin-2-yl)acetonitrile

To a solution of 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridin-2-yl)acetonitrile (40 mg, 0.088 mmol) in DMF (1 mL) at 80° C. was added Selectfluor (47 mg, 0.132 mmol) in DMF (1 mL) and CH₃CN (1 mL). The mixture was stirred at 80° C. for 5 mins. Then the reaction solvent was concentrated under reduced pressure, to obtain Compound A194. The resulting residue containing Compound A194 was further purified to give the titled Compound A194a (7 mg) and Compound A194b (3 mg) by Prep-HPLC (Method A).

Compound A194a (the earlier peak): ¹H NMR (400 MHz, CDCl₃) δ 11.23 (s, 1H), 8.85 (s, 2H), 8.13-7.93 (m, 3H), 7.52 (s, 1H), 5.11 (s, 2H), 4.49-4.51 (m, 1H), 4.11-3.86 (m, 2H), 3.72-3.54 (m, 1H), 3.10-3.07 (m, 1H), 2.81-2.62 (m, 1H), 2.41-2.37 (m, 1H), 2.06-2.02 (m, 1H), 1.90-1.76 (m, 1H), 1.47-1.43 (m, 3H), 1.27-1.23 (m, 2H), 1.23-0.86 (m, 2H), 0.75-0.54 (m, 2H) ppm. MS: M/e 475 (M+1)⁺.

Compound A194b (the later peak): ¹H NMR (400 MHz, CDCl₃) δ 11.13 (s, 1H), 8.84 (s, 2H), 8.10-7.99 (m, 3H), 7.52 (s, 1H), 5.12 (s, 2H), 4.51-4.11 (m, 1.5H), 4.90-3.50 (m, 2H), 3.13-2.83 (m, 1.5H), 2.20-2.16 (m, 1H), 1.83-1.79 (m, 2H), 1.44-1.40 (m, 3H), 1.28-1.25 (m, 4H), 1.00-0.96 (m, 3H) ppm. MS: M/e 475 (M+1)⁺.

### Compound A196: 2-(7-((2S,5R)-2,5-diethyl-4-(1-(4-fluoro-2-(trifluoromethyl)phenyl)ethyl)piperazin-1-yl)-4-methyl-5-oxo-4,5-dihydro-2H-pyrazolo[4,3-b]pyridin-2-yl)acetonitrile

### Step A: 1-(6-(difluoromethoxy)pyridin-3-yl)ethan-1-ol

To a solution of 1-(4-fluoro-2-(trifluoromethyl)phenyl)ethan-1-one (412 mg, 2 mmol) in MeOH (10 mL) was added NaBH₄ (80 mg, 2 mmol) at 0° C. The reaction was stirred at room temperature for 30 min. The reaction was quenched by H₂O (2 mL). The reaction solvent was removed under vacuum. The resulting residue was purified by flash column chromatography (PE:EA=10:1) to give the titled compound (380 mg, 92%). MS: M/e 209 (M+1)⁺.

### Step B: tert-butyl (2S,5R)-2,5-diethyl-4-(1-(4-fluoro-2-(trifluoromethyl)phenyl)ethyl)piperazine-1-carboxylate

A mixture of tert-butyl (2S,5R)-2,5-diethylpiperazine-1-carboxylate (240 mg, 1 mmol), 1-(6-(difluoromethoxy)pyridin-3-yl)ethan-1-ol (315 mg, 1.5 mmol), (cyanomethyl)trimethylphosphonium iodide (394 mg, 1.5 mmol) and DIPEA (650 mg, 5 mmol) in CH₃CN (5 mL) was heated to 105° C. overnight under N₂ atmosphere in a sealed tube. The reaction solvent was removed under vacuum. The resulting residue was purified by flash column chromatography (DCM:MeOH=100:1) to give the titled compound (180 mg, 42%). MS: M/e 433 (M+1)⁺.

### Step C: (2R,5S)-2,5-diethyl-1-(1-(4-fluoro-2-(trifluoromethyl)phenyl)ethyl)piperazine

To a solution of tert-butyl (2S,5R)-2,5-diethyl-4-(1-(2-(trifluoromethyl)phenyl)ethyl)piperazine-1-carboxylate (180 mg, 0.42 mmol) in DCM (5 mL) were added TFA (2 mL). The resulting mixture was stirred at RT for another 2 hours. The reaction solvent was removed under vacuum. The resulting residue was dissolved in DCM (10 mL). The organic layer was washed with NaOH aq. (1 M), dried over Na₂SO₄ and concentrated to give the titled compound (110 mg, 79%), which was used to the next step directly without further purification. MS: M/e 333 (M+1)⁺.

### Step D: 7-((2S,5R)-2,5-diethyl-4-(1-(4-fluoro-2-(trifluoromethyl)phenyl)ethyl)piperazin-1-yl)-4-methyl-2-(tetrahydro-2H-pyran-2-yl)-2,4-dihydro-5H-pyrazolo[4,3-b]pyridin-5-one

A mixture of Intermediate 2 (150 mg, 0.39 mmol), (2R,5S)-2,5-diethyl-1-(1-(4-fluoro-2-(trifluoromethyl)phenyl)ethyl)piperazine (110 mg, 0.33 mmol) and DIPEA (260 mg, 2 mmol) in CH₃CN was heated to 105° C. overnight under N₂ atmosphere. The reaction solvent was removed under vacuum. The resulting residue was purified by Prep-TLC (DCM:MeOH=15:1) to give the titled compound (80 mg, 44%). MS: M/e 564 (M+1)⁺.

### Step E: 7-((2S,5R)-2,5-diethyl-4-(1-(4-fluoro-2-(trifluoromethyl)phenyl)ethyl)piperazin-1-yl)-4-methyl-2,4-dihydro-5H-pyrazolo[4,3-b]pyridin-5-one

To a solution of 7-((2S,5R)-4-(1-(benzo[d]thiazol-2-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one (80 mg, 0.14 mmol) in MeOH (2 mL) was added HCl (0.5 mL, 2 mmol, 4M in dioxane) at room temperature. The resulting mixture was stirred at room temperature for another 2 hours. The reaction solvent was removed under vacuum. The crude product was dissolved in DCM (10 mL) and washed with NaOH aq. (1 M). The organic layer was dried and concentrated. The resulting residue was purified by Prep-TLC (DCM:MeOH=10:1) to give the titled compound (50 mg) as a mixture of diastereomers Compound A275, which was further separated into Compound A275a (2 mg) and Compound A275b (2 mg) by Prep-HPLC (Method A).

Compound A275a: ¹H NMR (400 MHz, DMSO-d6) δ 13.49 (s, 1H), 8.08-7.93 (m, 1H), 7.87 (s, 1H), 7.65-7.48 (m, 2H), 5.35 (s, 1H), 3.90 (s, 1H), 3.40-3.31 (m, 2H), 3.31-3.21 (m, 4H), 3.10 (d, J=9.2 Hz, 1H), 2.59 (d, J=10.8 Hz, 1H), 2.02 (d, J=12.0 Hz, 1H), 1.88-1.71 (m, 1H), 1.70-1.57 (m, 1H), 1.55-1.31 (m, 2H), 1.24 (d, J=6.4 Hz, 3H), 0.93 (t, J=7.2 Hz, 3H), 0.55-0.35 (m, 3H). MS: M/e 480 (M+1)⁺.

Compound A275b: ¹H NMR (400 MHz, DMSO-d6) δ 13.50 (s, 1H), 8.11-7.96 (m, 1H), 7.87 (s, 1H), 7.67-7.50 (m, 2H), 5.35 (s, 1H), 4.14-4.04 (m, 1H), 3.30-3.26 (m, 4H), 3.16-3.06 (m, 1H), 2.99 (d, J=11.6 Hz, 1H), 2.87-2.76 (m, 1H), 2.59-2.51 (m, 1H), 2.25-2.15 (m, 1H), 2.15-1.99 (m, 1H), 1.80-1.65 (m, 1H), 1.62-1.46 (m, 1H), 1.46-1.31 (m, 1H), 1.22 (d, J=6.4 Hz, 3H), 1.00-0.76 (m, 3H), 0.64 (t, J=7.2 Hz, 3H). MS: M/e 480 (M+1)⁺.

### Step F: 2-(7-((2S,5R)-2,5-diethyl-4-(1-(4-fluoro-2-(trifluoromethyl)phenyl)ethyl)piperazin-1-yl)-4-methyl-5-oxo-4,5-dihydro-2H-pyrazolo[4,3-b]pyridin-2-yl)acetonitrile

To a solution of 7-((2S,5R)-2,5-diethyl-4-(1-(4-fluoro-2-(trifluoromethyl)phenyl)ethyl)piperazin-1-yl)-4-methyl-2,4-dihydro-5H-pyrazolo[4,3-b]pyridin-5-one (40 mg, 0.08 mmol) and K₂CO₃ (24 mg, 0.17 mmol) in DMF (4 mL) were added 2-iodoacetonitrile (22 mg, 0.13 mmol). The resulting mixture was stirred at room temperature overnight. The reaction mixture was diluted with EA (10 mL) and washed with brine (10 mL×3). The organic layers were concentrated under reduced pressure. The resulting residue was purified by Prep-TLC (DCM:MeOH=15:1) to give the crude Compound A196. The crude Compound A196 was further purified to give Compound A196a (9 mg) and Compound A196b (9 mg) by Prep-HPLC (Method A).

Compound A196a (the earlier peak): ¹H NMR (400 MHz, CD₃OD) δ 8.07-8.05 (m, 1H), 7.93 (s, 1H), 7.42-7.39 (m, 2H), 5.55 (s, 1H), 5-47 (s, 2H), 4.03-4.02 (m, 1H), 3.52-3.48 (m, 1H), 3-43 (s, 3H), 3.30-3-19 (m, 2H), 2.85-2.68 (m, 2H), 2.16 (d, J=12.5 Hz, 1H), 1.93 (br s, 1H), 1.69-1.63 (m, 2H), 1.51-1.48 (m, 1H), 1.30 (d, J=6.4 Hz, 3H), 1.06-1.04 (m, 3H), 0.60-0.58 (m, 3H) ppm. MS: M/e 519 (M+1)⁺.

Compound A196b (the later peak): ¹H NMR (400 MHz, CD₃OD) δ 8.08-8.06 (m, 1H), 7.93 (s, 1H), 7.44-7.40 (m, 2H), 5.57 (s, 1H), 5-46 (s, 2H), 4.19-4.18 (m, 1H), 3-43 (s, 3H), 3.33-3-31 (m, 2H), 3.10-3-09 (m, 1H), 2.95-2.78 (m, 2H), 2.35-2.30 (m, 2H), 1.87-1.84 (m, 1H), 1.60-1.48 (m, 2H), 1.28 (d, J=6.4 Hz, 3H), 1.01-0.99 (m, 3H), 0.74-0.73 (m, 3H) ppm. MS: M/e 519 (M+1)⁺.

### Compound A200: 2-(cyanomethyl)-7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridine-6-carbonitrile

### Step A: 2-(6-bromo-7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridin-2-yl)acetonitrile

To a solution of 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridin-2-yl)acetonitrile (120 mg, 0.263 mmol) in CH₃CN (15 mL) at 0° C. was added NBS (47 mg, 0.263 mmol). The mixture was stirred at 0° C. for 30 mins. Then the mixture was quenched with saturated NH₄Cl (20 mL) and extracted with EA (30 mL×3). The combined organic layer was washed with brine (20 mL×3), dried over Na₂SO₄ and concentrated under reduced pressure. The resulting residue was purified by flash column chromatography to give the titled compound (60 mg, 43%). MS: M/e 535 (M+1)⁺.

### Step B: 2-(cyanomethyl)-7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridine-6-carbonitrile

To a solution of 2-(6-bromo-7-((2S,5R)-5-ethyl-2-methA97yl-4-(1-(quinoxalin-6-yl)ethyl)piperazin-1-yl)-5-oxo-4,5-dihydro-2H-pyrazolo[4,3-b]pyridin-2-yl)acetonitrile (42 mg, 0.079 mmol), Zn(CN)₂ (28 mg, 0.157 mmol) and Pd(PPh₃)₄ (37 mg, 0.032 mmol) in DMF (2 mL) was degassed 3 times under N₂ atmosphere. Then the mixture solution was stirred at 100° C. for 24 hours. The reaction mixture was quenched with saturated NH₄Cl (20 mL) at room temperature and extracted with EA (30 mL×2). The combined organic layer was dried over Na₂SO₄ and concentrated under reduced pressure, to obtain Compound A200. The resulting residue containing Compound A200 was further purified to give Compound A200a (2 mg, 5%) and Compound A200b (3 mg, 8%) by Prep-HPLC (Method A).

Compound A200a (the earlier peak): ¹H NMR (400 MHz, CDCl₃) δ 11.16 (s, 1H), 8.85 (s, 2H), 8.14-8.10 (m, 2H), 8.04-8.00 (m, 1H), 7.93-7.90 (m, 1H), 5.11 (s, 3H), 4.02 (s, 1H), 3.84 (s, 1H), 3.52 (s, 1H), 3.15-3.11 (m, 1H), 2.87-2.83 (m, 1H), 2.53-2.49 (m, 1H), 1.63-1.58 (m, 4H), 1.45-1.43 (m, 4H), 0.61 (s, 3H) ppm. MS: M/e 482 (M+1)⁺.

Compound A200b (the later peak): ¹H NMR (400 MHz, CDCl₃) δ 10.94 (s, 1H), 8.85-8.84 (m, 2H), 8.13-8.09 (m, 1H), 8.09-8.04 (m, 2H), 7.95-7.93 (m, 1H), 5.14-5.10 (m, 3H), 4.08 (s, 1H), 3.89-3.87 (m, 1H), 3.51-3.47 (m, 1H), 3.26-2.22 (m, 1H), 2.94-2.90 (m, 1H), 2.32-2.29 (m, 1H), 1.58-1.42 (m, 8H), 0.94 (s, 3H) ppm. MS: M/e 482 (M+1)⁺.

### Compound A202: 2-(7-((2S,5R)-5-ethyl-4-(1-(2-fluoro-4-(trifluoromethyl)phenyl)ethyl)-2-methylpiperazin-1-yl)-4-methyl-5-oxo-4,5-dihydro-2H-pyrazolo[4,3-b]pyridin-2-yl)acetonitrile

### Step A: tert-butyl (2S,5R)-5-ethyl-4-(1-(2-fluoro-4-(trifluoromethyl)phenyl)ethyl)-2-methylpiperazine-1-carboxylate

To a solution of 1-(2-fluoro-4-(trifluoromethyl)phenyl)ethan-1-ol (350 mg, 1.682 mmol), tert-butyl (2S,5R)-5-ethyl-2-methylpiperazine-1-carboxylate (462 mg, 2.020 mmol) and (cyanomethyl)trimethylphosphonium iodide (613 mg, 2.523 mmol) in CH₃CN (2 mL) was added DIPEA (1085 mg, 8.410 mmol). The mixture solution was degassed 3 times under N₂ atmosphere. Then the reaction mixture was stirred at 105° C. for 24 hours. The reaction mixture was quenched with saturated NH₄Cl (20 mL) at room temperature and extracted with EA (30 mL×2). The combined organic layer was dried over Na₂SO₄ and concentrated under reduced pressure. The resulting residue was purified by flash column chromatography to give the titled compound (350 mg, 50%). MS: M/e 419 (M+1)⁺.

### Step B: (2R,5S)-2-ethyl-1-(1-(2-fluoro-4-(trifluoromethyl)phenyl)ethyl)-5-methylpiperazine

To a solution of tert-butyl (2S,5R)-5-ethyl-4-(1-(2-fluoro-4-(trifluoromethyl)phenyl)ethyl)-2-methylpiperazine-1-carboxylate (250 mg, 0.598 mmol) in DCM (15 mL) at room temperature was added HCl (5 mL, 4 M in 1,4-dioxane). The mixture was stirred at room temperature for 2 hours. Then the mixture was concentrated under reduced pressure to give the crude product (HCl salt). The crude product was dissolved in water. The aqueous was basified to pH ˜10 with Na₂CO₃ aq. (4M) and extracted with EA (30 mL×3). The combined organic layer was washed with brine (20 mL×3), dried over Na₂SO₄ and concentrated under reduced pressure to give the titled compound (170 mg, 89%). MS: M/e 319 (M+1)⁺.

### Step C: 7-((2S,5R)-5-ethyl-4-(1-(2-fluoro-4-(trifluoromethyl)phenyl)ethyl)-2-methylpiperazin-1-yl)-4-methyl-2-(tetrahydro-2H-pyran-2-yl)-2,4-dihydro-5H-pyrazolo[4,3-b]pyridin-5-one

To a solution of (2R,5S)-2-ethyl-1-(1-(2-fluoro-4-(trifluoromethyl)phenyl)ethyl)-5-methylpiperazine (160 mg, 0.503 mmol) and Intermediate 2 (210 mg, 0.553 mmol) in CH₃CN (2 mL) at room temperature was added DIPEA (130 mg, 1.006 mmol). The mixture was stirred at 105° C. for 24 hours. Then the mixture was concentrated under reduced pressure. The resulting residue was purified by flash column chromatography to give the titled compound (200 mg, 72%). MS: M/e 550 (M+1)⁺.

### Step D: 7-((2S,5R)-5-ethyl-4-(1-(2-fluoro-4-(trifluoromethyl)phenyl)ethyl)-2-methylpiperazin-1-yl)-4-methyl-2,4-dihydro-5H-pyrazolo[4,3-b]pyridin-5-one

To a solution of 7-((2S,5R)-5-ethyl-4-(1-(2-fluoro-4-(trifluoromethyl)phenyl)ethyl)-2-methylpiperazin-1-yl)-4-methyl-2-(tetrahydro-2H-pyran-2-yl)-2,4-dihydro-5H-pyrazolo[4,3-b]pyridin-5-one (150 mg, 0.273 mmol) in MeOH (10 mL) at room temperature was added HCl (4 mL, 4 M in 1,4-dioxane). The mixture was stirred at room temperature for 2 hours. Then the reaction mixture was concentrated under reduced pressure. The resulting residue was dissolved in water. The aqueous was basified to pH ˜10 with Na₂CO₃ aq. (4M) and extracted with EA (35 mL×3). The combined organic layer was washed with brine (20 mL×3), dried over Na₂SO₄ and concentrated under reduced pressure to give the titled compound (100 mg, 79%). MS: M/e 466 (M+1)⁺.

### Step E: 2-(7-((2S,5R)-5-ethyl-4-(1-(2-fluoro-4-(trifluoromethyl)phenyl)ethyl)-2-methylpiperazin-1-yl)-4-methyl-5-oxo-4,5-dihydro-2H-pyrazolo[4,3-b]pyridin-2-yl)acetonitrile

To a solution of 7-((2S,5R)-5-ethyl-4-(1-(2-fluoro-4-(trifluoromethyl)phenyl)ethyl)-2-methylpiperazin-1-yl)-4-methyl-2,4-dihydro-5H-pyrazolo[4,3-b]pyridin-5-one (80 mg, 0.172 mmol) and K₂CO₃ (47 mg, 0.344 mmol) in DMF (8 mL) at room temperature was added 2-iodoacetonitrile (43 mg, 0.258 mmol). The mixture solution was stirred at room temperature for 12 hours. Then the reaction mixture was quenched with saturated NaCl (20 mL) at room temperature and extracted with EA (30 mL×2). The combined organic layer was dried over Na₂SO₄ and concentrated under reduced pressure, to obtain Compound A202. The resulting residue containing Compound A202 was purified by Prep-HPLC (Method A) to give Compound A202a (70 mg) and Compound A202b (141 mg).

Compound A202a (the earlier peak): ¹H NMR (400 MHz, CDCl₃) δ 7.71-7.67 (m, 1H), 7.43 (d, J=8 Hz, 1H), 7.39 (s, 1H), 7.31 (d, J=8 Hz, 1H), 5.64-5.62 (m, 1H), 5.13 (s, 2H), 4.68-4.40 (m, 1H), 4.24-4.22 (m, 1H), 3.43 (s, 3H), 3.31-3.27 (m, 1H), 2.98-2.94 (m, 1H), 2.77-2.74 (m, 1H), 2.38-2.36 (m, 1H), 2.05-1.86 (m, 1H), 1.67-1.63 (m, 1H), 1.65-1.60 (m, 1H), 1.52-1.49 (m, 6H), 0.75 (m, 3H) ppm. MS: M/e 505 (M+1)⁺.

Compound A202b (the later peak): ¹H NMR (400 MHz, CDCl₃) δ 7.81-7.77 (m, 1H), 7.42-7.40 (m, 2H), 7.29-7.27 (m, 1H), 5.65 (s, 1H), 5.19-5.15 (m, 2H), 4.78-4.51 (m, 2H), 4.07-4.03 (m, 1H), 3.46-3.42 (m, 4H), 3.09-3.04 (m, 1H), 2.82-2.78 (m, 1H), 2.16-2.13 (m, 1H), 1.78-1.73 (m, 1H), 1.51-1.47 (m, 1H), 1.22-1.18 (m, 3H), 1.06-1.02 (m, 3H), 1.03-0.98 (m, 3H) ppm. MS: M/e 505 (M+1)⁺.

### Compound A205: 2-(7-((2S,5R)-2,5-diethyl-4-(1-(2-ethyl-4-fluorophenyl)ethyl)piperazin-1-yl)-4-methyl-5-oxo-4,5-dihydro-2H-pyrazolo[4,3-b]pyridin-2-yl)acetonitrile

### Step A: 1-(4-fluoro-2-vinylphenyl)ethan-1-ol

To a solution of 1-(4-fluoro-2-vinylphenyl)ethan-1-one (300 mg, 1.829 mmol) in DCM (10 mL) was added sodium borohydride (139 mg, 3.658 mmol) at 0° C. and stirred at RT for 1 hours. The reaction mixture was quenched with MeOH, extracted with DCM (100 mL) and washed with water. The organic layer was dried and concentrated to dryness. The resulting residue was purified by flash column chromatography (PE:EA=1:1) to give the titled compound (290 mg, 96%).

### Step B: 1-(2-ethyl-4-fluorophenyl)ethan-1-ol

A mixture of 1-(4-fluoro-2-vinylphenyl)ethan-1-ol (290 mg, 1.074 mmol), Pd/C (20 mg, 10% in water) in MeOH (20 mL) was stirred under H₂ atmosphere (1 atm) at RT overnight. The reaction mixture was filtered and concentrated to give the titled compound (290 mg, 99%).

### Step C: 7-((2S,5R)-2,5-diethyl-4-(1-(2-ethyl-4-fluorophenyl)ethyl)piperazin-1-yl)-4-methyl-2-(tetrahydro-2H-pyran-2-yl)-2,4-dihydro-5H-pyrazolo[4,3-b]pyridin-5-one

A mixture of Intermediate 3 (100 mg, 0.2681 mmol), 1-(2-ethyl-4-fluorophenyl)ethan-1-ol (90 mg, 0.5362 mmol), (cyanomethyl)trimethylphosphonium iodide (130 mg, 0.5362 mmol), DIPEA (173 mg, 1.340 mmol) in CH₃CN (5 mL) was stirred under N₂ at 105° C. overnight. The mixture was extracted with DCM and washed with water. The organic layer was dried and concentrated to dryness. The resulting residue was purified by Prep-TLC (DCM:MeOH=20:1) to give the titled compound (80 mg, 57%). MS: M/e 524 (M+1)⁺

### Step D: 7-((2S,5R)-2,5-diethyl-4-(1-(2-ethyl-4-fluorophenyl)ethyl)piperazin-1-yl)-4-methyl-2,4-dihydro-5H-pyrazolo[4,3-b]pyridin-5-one

A mixture of 7-((2S,5R)-2,5-diethyl-4-(1-(2-ethyl-4-fluorophenyl)ethyl)piperazin-1-yl)-4-methyl-2-(tetrahydro-2H-pyran-2-yl)-2,4-dihydro-5H-pyrazolo[4,3-b]pyridin-5-one (80 mg, 0.1487 mmol) in TFA (2 mL) and DCM (2 mL) was stirred at RT overnight. The reaction solvent was concentrated and the resulting residue was purified by Prep-TLC (DCM:MeOH=20:1) to give the titled compound (48 mg, 71.48%). MS: M/e 440 (M+1)⁺

### Step E: 2-(7-((2S,5R)-2,5-diethyl-4-(1-(2-ethyl-4-fluorophenyl)ethyl)piperazin-1-yl)-4-methyl-5-oxo-4,5-dihydro-2H-pyrazolo[4,3-b]pyridin-2-yl)acetonitrile

A mixture of 7-((2S,5R)-2,5-diethyl-4-(1-(2-ethyl-4-fluorophenyl)ethyl)piperazin-1-yl)-4-methyl-2,4-dihydro-5H-pyrazolo[4,3-b]pyridin-5-one (48 mg, 0.1093 mmol), 2-iodoacetonitrile (37 mg, 0.2187 mmol), K₂CO₃ (45 mg, 0.3280 mmol) in DMF (3 mL) was stirred at RT for 5 hours. The mixture was extracted with DCM (50 mL) and washed with water. The organic layer was dried and concentrated to dryness. The resulting residue was purified by Prep-TLC (DCM:MeOH=20:1) to give the titled compound (12 mg, 23%).

¹H NMR (400 MHz, CD₃OD) δ 7.93 (s, 1H), 7.67-7.48 (m, 1H), 7.05-6.73 (m, 2H), 5.56 (s, 1H), 5.47 (s, 2H), 4.03-3.89 (m, 1H), 3.43 (s, 3H), 2.92-2.60 (m, 5H), 2.46-2.08 (m, 2H), 1.88-1.39 (m, 4H), 1.36-1.18 (m, 7H), 1.07-0.91 (m, 3H), 0.74-0.55 (m, 3H) ppm. MS: M/e 479 (M+1)⁺

### Compound A206: 2-(7-((2S,5R)-2,5-diethyl-4-(3-(trifluoromethyl)benzoyl)piperazin-1-yl)-4-methyl-5-oxo-4,5-dihydro-2H-pyrazolo[4,3-b]pyridin-2-yl)acetonitrile

### Step A: 7-((2S,5R)-2,5-diethyl-4-(3-(trifluoromethyl)benzoyl)piperazin-1-yl)-4-methyl-2-(tetrahydro-2H-pyran-2-yl)-2,4-dihydro-5H-pyrazolo[4,3-b]pyridin-5-one

A mixture of Intermediate 3 (20 mg, 0.0536 mmol), 3-(trifluoromethyl)benzoic acid (15 mg, 0.0804 mmol), HATU (31 mg, 0.0804 mmol) and TEA (16 mg, 0.1609 mmol) in DCM (5 mL) was stirred at RT for 2 hours. The mixture was extracted with DCM (10 mL) and washed with water (5 ml), dried over Na₂SO₄ and concentrated to dryness. The resulting residue was purified with Prep-HPLC to give the titled compound (25 mg, 86%). MS: M/e 546 (M+1)⁺

### Step B: 7-((2S,5R)-2,5-diethyl-4-(3-(trifluoromethyl)benzoyl)piperazin-1-yl)-4-methyl-2,4-dihydro-5H-pyrazolo[4,3-b]pyridin-5-one

A mixture of 7-((2S,5R)-2,5-diethyl-4-(3-(trifluoromethyl)benzoyl)piperazin-1-yl)-4-methyl-2-(tetrahydro-2H-pyran-2-yl)-2,4-dihydro-5H-pyrazolo[4,3-b]pyridin-5-one (25 mg, 0.046 mmol) in TFA (2 mL) and DCM (2 mL) was stirred at RT overnight. The reaction solvent was concentrated and purified by Prep-TLC (DCM:MeOH=20:1) to give the titled compound (15 mg, 71%). MS: M/e 462 (M+1)⁺

### Step C: 2-(7-((2S,5R)-2,5-diethyl-4-(3-(trifluoromethyl)benzoyl)piperazin-1-yl)-4-methyl-5-oxo-4,5-dihydro-2H-pyrazolo[4,3-b]pyridin-2-yl)acetonitrile

A mixture of 7-((2S,5R)-2,5-diethyl-4-(3-(trifluoromethyl)benzoyl)piperazin-1-yl)-4-methyl-2,4-dihydro-5H-pyrazolo[4,3-b]pyridin-5-one (15 mg, 0.032 mmol), 2-iodoacetonitrile (8 mg, 0.049 mmol), K₂CO₃ (9 mg, 0.065 mmol) in DMF (3 mL) was stirred at RT for 5 hours. The reaction mixture was quenched with water (20 mL), extracted with DCM (50 mL). The organic layer was washed with brine, dried and concentrated to dryness. The resulting residue was purified by Prep-TLC (DCM:MeOH=20:1) to give the titled compound (10 mg, 61%). ¹H NMR (400 MHz, CD₃OD) δ 7.96 (d, J=2.6 Hz, 1H), 7.83 (d, J=6.6 Hz, 1H), 7.78-7.65 (m, 3H), 5.64 (d, J=10.8 Hz, 1H), 5.49 (s, 2H), 4.86-4.52 (m, 2H), 3.70 (d, J=9.6 Hz, 1H), 3.54 (d, J=14.3 Hz, 1H), 3.48 (s, 1H), 3.44 (d, J=2.2 Hz, 3H), 2.91-2.68 (m, 1H), 1.94-1.65 (m, 4H), 1.08-0.98 (m, 3H), 0.82-0.63 (m, 3H) ppm. MS: M/e 501 (M+1)⁺

### Compound A209: 2-(7-((2S,5R)-2,5-dimethyl-4-(1-(quinoxalin-6-yl)ethyl)piperazin-1-yl)-3,4-dimethyl-5-oxo-4,5-dihydro-2H-pyrazolo[4,3-b]pyridin-2-yl)acetonitrile

### Step A: 5-methyl-4-nitro-1H-pyrazole-3-carboxylic acid

To a solution of conc. HNO₃ (6 mL) at 0° C. was added conc. H₂SO₄ (6 mL) dropwise. Then the resulting solution was heated 50° C. and 5-methyl-1H-pyrazole-3-carboxylic acid (4 g, 31.74 mmol) was added in some portions to keep the temperature below 60° C. The reaction was stirred at 60° C. for 18 hours. Then the mixture was cooled to room temperature, poured into ice-water. A suspension was formed and filtered. The filter cake was dried to give the titled compound (5.06 g, 93%). MS: M/e 172 (M+23)⁺.

### Step B: ethyl 5-methyl-4-nitro-1H-pyrazole-3-carboxylate

To a solution of 5-methyl-4-nitro-1H-pyrazole-3-carboxylic acid (5.06 g, 29.59 mmol) in EtOH (20 mL) was added conc. H₂SO₄ (2 mL). The reaction was stirred at 70° C. for 18 hours. The mixture was concentrated to dryness, diluted with EA (80 mL), washed with water, brine, dried over Na₂SO₄, filtered, and concentrated to give titled compound (5.75 g, 97%). MS: M/e 200 (M+1)⁺.

### Step C: ethyl 5-methyl-4-nitro-1-(tetrahydro-2H-pyran-2-yl)-1H-pyrazole-3-carboxylate

To a solution of ethyl 5-methyl-4-nitro-1H-pyrazole-3-carboxylate (5.85 g, 29.39 mmol) and TsOH·H₂O (560 mg, 2.94 mmol) in THF (40 mL) was added DHP (4.93 g, 58.8 mmol). The reaction was stirred at 80° C. for 16 hours. Then the reaction mixture was cooled to room temperature and concentrated to dryness. The resulting residue was purified by flash column chromatography (EA:PE=1:5) to give titled compound (4.6 g, 55%). ¹HNMR (400 MHz, CDCl₃) δ 5.41 (d, J=8.0 Hz, 1H), 4.49-4.37 (m, 2H), 4.04-3.92 (m, 1H), 3.74-3.62 (m, 1H), 2.68 (s, 3H), 2.46-2.35 (m, 1H), 2.23-2.08 (m, 1H), 2.06-1.95 (m, 1H), 1.74-1.62 (m, 3H), 1.42-1.34 (m, 3H) ppm.

### Step D: ethyl 4-amino-5-methyl-1-(tetrahydro-2H-pyran-2-yl)-1H-pyrazole-3-carboxylate

To a solution of ethyl 5-methyl-4-nitro-1-(tetrahydro-2H-pyran-2-yl)-1H-pyrazole-3-carboxylate (4.6 g, 16.25 mmol) in MeOH (50 mL) was added 10% of Pd/C (460 mg, 10% in water). The mixture was stirred at room temperature under H₂ atmosphere (balloon) overnight. The mixture was filtered, washed with MeOH. The combined filtrate was concentrated to give titled compound (3.8 g, 92%). MS: M/e 254 (M+1)⁺.

### Step E: Ethyl 4-acetamido-5-methyl-1-(tetrahydro-2H-pyran-2-yl)-1H-pyrazole-3-carboxylate

To a solution of ethyl 4-amino-5-methyl-1-(tetrahydro-2H-pyran-2-yl)-1H-pyrazole-3-carboxylate (3.8 g, 15.01 mmol) in pyridine (50 mL) was added Ac₂O (10 mL) dropwise to keep the temperature below 20° C.

The resulting mixture was stirred at RT for 1 hours. The mixture was concentrated to dryness. The resulting residue was subjected to slurry with PE/EA to give titled compound (3.8 g, 85%). MS: M/e 296 (M+1)⁺.

### Step F: Ethyl 5-methyl-4-(N-methylacetamido)-1-(tetrahydro-2H-pyran-2-yl)-1H-pyrazole-3-carboxylate

To a solution of Ethyl 4-acetamido-5-methyl-1-(tetrahydro-2H-pyran-2-yl)-1H-pyrazole-3-carboxylate (3.8 g, 12.88 mmol) in DMF (30 mL) was added Cs₂CO₃ (12.6 g, 38.6 mmol), followed by CH₃I (5.5 g, 38.6 mmol). The reaction was stirred at RT for 16 hours. The mixture was diluted with water, extracted with EA (80 mL×3), washed with brine, dried over Na₂SO₄, filtered, and concentrated to dryness.

The resulting residue was purified by flash column chromatography (EA:PE=1:1) to give titled compound (3.85 g, 96%). MS: M/e 310 (M+1)⁺.

### Step G: 7-hydroxy-3,4-dimethyl-2-(tetrahydro-2H-pyran-2-yl)-2,4-dihydro-5H-pyrazolo[4,3-b]pyridin-5-one

To a solution of Ethyl 5-methyl-4-(N-methylacetamido)-1-(tetrahydro-2H-pyran-2-yl)-1H-pyrazole-3-carboxylate (618 mg, 2 mmol) in THF (20 mL) was added dropwise a solution of L1HMDS (4 mL, 1 mol/L) at −70° C. The reaction mixture was stirred for 2 hours, quenched with saturated citric acid solution to pH 3-4, extracted with DCM:IPA (5:1, 60 mL×3), dried over Na₂SO₄, filtered, and concentrated to dryness. The resulting residue was purified by flash column chromatography (DCM:MeOH=10:1) to give titled compound (100 mg, 19%). ¹HNMR (400 MHz, DMSO-d₆) δ 11.08 (s, 1H), 5.63 (s, 1H), 5.51 (dd, J=2.4 Hz, 9.6 Hz, 1H), 3.93-3.82 (m, 1H), 3.74-3.61 (m, 1H), 3.51 (s, 3H), 2.62 (s, 3H), 2.42-2.24 (m, 1H), 2.10-1.96 (m, 1H), 1.94-1.84 (m, 1H), 1.76-1.62 (m, 1H), 1.59-1.48 (m, 2H) ppm. MS: M/e 264 (M+1)⁺.

### Step H: 3,4-dimethyl-5-oxo-2-(tetrahydro-2H-pyran-2-yl)-4,5-dihydro-2H-pyrazolo[4,3-b]pyridin-7-yl trifluoromethanesulfonate

To a solution of 7-hydroxy-3,4-dimethyl-2-(tetrahydro-2H-pyran-2-yl)-2,4-dihydro-5H-pyrazolo[4,3-b]pyridin-5-one (100 mg, 0.38 mmol) in THF (5 mL) was added K₂CO₃ (105 mg, 0.76 mmol), followed by 1,1,1-trifluoro-N-phenyl-N-((trifluoromethyl)sulfonyl)methanesulfonamide (203 mg, 0.57 mmol). The reaction was stirred at room temperature for 4 hours. The mixture was diluted with water, extracted with EA (80 mL), washed with brine, dried over Na₂SO₄, filtered, and concentrated to dryness. The resulting residue was purified by flash column chromatography (EA:PE=1:2) to give titled compound (85 mg, 90%). MS: M/e 396 (M+1)⁺.

### Step I: 7-((2S,5R)-2,5-dimethyl-4-(1-(quinoxalin-6-yl)ethyl)piperazin-1-yl)-3,4-dimethyl-2-(tetrahydro-2H-pyran-2-yl)-2,4-dihydro-5H-pyrazolo[4,3-b]pyridin-5-one

To a solution of 3,4-dimethyl-5-oxo-2-(tetrahydro-2H-pyran-2-yl)-4,5-dihydro-2H-pyrazolo[4,3-b]pyridin-7-yl trifluoromethanesulfonate (85 mg, 0.215 mmol) and Intermediate 1 (70 mg, 0.258 mmol) in CH₃CN (3 mL) was added DIPEA (55 mg, 0.43 mmol). Then the mixture was heated at 100° C. under N₂ for 48 hours. The mixture was cooled to room temperature, diluted with water (50 mL), extracted with EA (60 mL×2), washed with brine, dried over Na₂SO₄, filtered and concentrated. The resulting residue was purified by flash column chromatography (DCM:MeOH=15:1) to give titled compound (65 mg, 59%). MS: M/e 516 (M+1)⁺.

### Step J: 7-((2S,5R)-2,5-dimethyl-4-(1-(quinoxalin-6-yl)ethyl)piperazin-1-yl)-3,4-dimethyl-2,4-dihydro-5H-pyrazolo[4,3-b]pyridin-5-one

To a solution of 7-((2S,5R)-2,5-dimethyl-4-(1-(quinoxalin-6-yl)ethyl)piperazin-1-yl)-3,4-dimethyl-2-(tetrahydro-2H-pyran-2-yl)-2,4-dihydro-5H-pyrazolo[4,3-b]pyridin-5-one (65 mg, 0.126 mmol) in DCM (3 mL) was added TFA (6 mL). The resulting mixture was stirred at room temperature overnight. The reaction mixture was concentrated under reduced pressure, diluted with water/DCM, basified with saturated NaHCO₃ solution to pH 7-8, extracted with DCM (60 mL×2). The combined organic layers were washed with brine, dried over Na₂SO₄, filtered and concentrated. The resulting residue was purified by flash column chromatography (DCM:MeOH=12: 1) to give titled compound (28 mg, 51%). MS: M/e 432 (M+1)⁺.

### Step K: 2-(7-((2S,5R)-2,5-dimethyl-4-(1-(quinoxalin-6-yl)ethyl)piperazin-1-yl)-3,4-dimethyl-5-oxo-4,5-dihydro-2H-pyrazolo[4,3-b]pyridin-2-yl)acetonitrile

To a solution of 7-((2S,5R)-2,5-dimethyl-4-(1-(quinoxalin-6-yl)ethyl)piperazin-1-yl)-3,4-dimethyl-2,4-dihydro-5H-pyrazolo[4,3-b]pyridin-5-one (28 mg, 0.065 mmol) and K₂CO₃ (18 mg, 0.138 mmol) in DMF (2 mL) was added 2-iodoacetonitrile (16 mg, 0.0975 mmol). The reaction was stirred at room temperature overnight. The reaction mixture was diluted with water, extracted with EA (60 mL×2), washed with brine, dried over Na₂SO₄, filtered, and concentrated to dryness. The resulting residue was purified by Prep-TLC ((DCM:MeOH=15:1) to give titled compound (4 mg, 12%). ¹HNMR (400 MHz, CD₃OD) δ 8.92-8.84 (m, 2H), 8.21-8.00 (m, 3H), 5.57 (s, 1H), 5.46 (d, J=3.6 Hz, 2H), 4.75-4.45 (m, 1H), 4.35-4.15 (m, 0.5H), 4.05-3.75 (m, 1H), 3.72-3.64 (m, 1H), 3.65 (s, 3H), 3.50-3.40 (m, 0.5H), 3.35-3.31 (m, 0.5H), 3.16-2.80 (m, 2H), 2.73 (s, 3H), 2.25-2.20 (m, 0.5H), 1.62-1.35 (m, 4.5H), 1.22 (d, J=6.8 Hz, 3H), 1.14-1.00 (m, 1.5H) ppm. MS: M/e 471 (M+1)⁺.

### Compound A218: 2-(7-((2S,5R)-5-ethyl-4-(1-(4-fluoro-2-(trifluoromethyl)phenyl)ethyl)-2-methylpiperazin-1-yl)-4-methyl-5-oxo-4,5-dihydro-2H-pyrazolo[4,3-b]pyridin-2-yl)acetonitrile

### Step A: 7-((2S,5R)-5-ethyl-2-methylpiperazin-1-yl)-4-methyl-2-(tetrahydro-2H-pyran-2-yl)-2,4-dihydro-5H-pyrazolo[4,3-b]pyridin-5-one

Intermediate 11A (11.5 g, 25.05 mmol) was added into a solution of TFA in DCM (1:5, 100 mL). The resulting mixture was stirred at room temperature for 5 hours. The reaction mixture was quenched with saturated NaHCO₃ aq. (200 mL) and extracted with DCM (50 mL×3). The combined organic layers were washed with brine (100 mL×2), dried over Na₂SO₄ and concentrated to dryness to give the titled compound (8.4 g, 93%). MS: M/e 360 (M+1)⁺.

### Step B: 7-((2S,5R)-5-ethyl-4-(1-(4-fluoro-2-(trifluoromethyl)phenyl)ethyl)-2-methylpiperazin-1-yl)-4-methyl-2-(tetrahydro-2H-pyran-2-yl)-2,4-dihydro-5H-pyrazolo[4,3-b]pyridin-5-one

A mixture of 7-((2S,5R)-5-ethyl-2-methylpiperazin-1-yl)-4-methyl-2-(tetrahydro-2H-pyran-2-yl)-2,4-dihydro-5H-pyrazolo[4,3-b]pyridin-5-one (100 mg, 0.27 mmol), 1-(4-fluoro-2-(trifluoromethyl)phenyl)ethan-1-ol (150 mg, 0.72 mmol), (cyanomethyl)trimethylphosphonium iodide (270 mg, 1.11 mmol) and DIPEA (250 mg, 1.95 mmol) in MeCN (2 mL) was stirred at 100° C. for 24 hours. The resulting mixture was diluted with EA (5 mL), washed with brine (2 mL×3), dried and concentrated to dryness. The resulting residue was purified by flash column chromatography to give the titled compound (45 mg, 29%). MS: M/e 550 (M+1)⁺.

### Step C: 7-((2S,5R)-5-ethyl-4-(1-(4-fluoro-2-(trifluoromethyl)phenyl)ethyl)-2-methylpiperazin-1-yl)-4-methyl-2,4-dihydro-5H-pyrazolo[4,3-b]pyridin-5-one

To a solution of 7-((2S,5R)-5-ethyl-4-(1-(4-fluoro-2-(trifluoromethyl)phenyl)ethyl)-2-methylpiperazin-1-yl)-4-methyl-2-(tetrahydro-2H-pyran-2-yl)-2,4-dihydro-5H-pyrazolo[4,3-b]pyridin-5-one (40 mg, 0.07 mmol) in MeOH (2 mL) was added HCl (2 mL, 4M in 1,4-dioxane) at room temperature and the resulting mixture was stirred at room temperature for 2 hours. The reaction mixture was quenched with saturated NaHCO₃ aq. and extracted with DCM (2 mL×3). The combined organic layers were washed with brine (2 mL×2), dried over Na₂SO₄, and concentrated to give the titled compound (25 mg, 75%). MS: M/e 466 (M+1)⁺.

### Step D: 2-(7-((2S,5R)-5-ethyl-4-(1-(4-fluoro-2-(trifluoromethyl)phenyl)ethyl)-2-methylpiperazin-1-yl)-4-methyl-5-oxo-4,5-dihydro-2H-pyrazolo[4,3-b]pyridin-2-yl)acetonitrile

To a solution of 7-((2S,5R)-5-ethyl-4-(1-(4-fluoro-2-(trifluoromethyl)phenyl)ethyl)-2-methylpiperazin-1-yl)-4-methyl-2,4-dihydro-5H-pyrazolo[4,3-b]pyridin-5-one (25 mg, 0.05 mmol) and K₂CO₃ (55 mg, 0.40 mmol) in MeCN (1 mL) was added 2-iodoacetonitrile (25 mg, 0.15 mmol). The resulting mixture was stirred at room temperature for 16 hours. The reaction mixture was diluted with EA (10 mL), washed with brine (10 mL×3), dried over Na₂SO₄, and concentrated to dryness, to obtain Compound A218. The resulting residue containing Compound A218 was purified by flash column chromatography and further separated into Compound A218a (2 mg) and Compound A218b (4 mg) by Prep-HPLC (Method B).

Compound A218a (the earlier peak): ¹H NMR (400 MHz, CD₃OD) δ 8.19-8.04 (m, 1H), 7.93 (s, 1H), 7.51-7.34 (m, 2H), 5.59 (s, 1H), 5.46 (s, 2H), 4.28-4.07 (m, 1H), 3.50-3.39 (m, 3H), 3.29-3.25 (m, 2H), 3.03-2.90 (m, 2H), 2.90-2.72 (m, 1H), 2.33 (d, J=9.6 Hz, 1H), 1.72-1.57 (m, 1H), 1.56-1.48 (m, 1H), 1.46 (d, J=6.5 Hz, 3H), 1.27 (d, J=6.4 Hz, 3H), 0.77 (t, J=7.2 Hz, 3H) ppm. MS: M/e 505 (M+1)⁺.

Compound A218b (the later peak): ¹H NMR (400 MHz, CD₃OD) δ 8.13-8.03 (m, 1H), 7.93 (s, 1H), 7.47-7.31 (m, 2H), 5.58 (s, 1H), 5.47 (s, 2H), 4.12-3.96 (m, 1H), 3.53 (d, J=12.0 Hz, 1H), 3.43 (s, 3H), 3.33-3.31 (m, 2H), 3.21 (d, J=10.8 Hz, 1H), 2.84-2.74 (m, 1H), 2.02 (d, J=12.0 Hz, 1H), 1.85-1.66 (m, 1H), 1.58-1.44 (m, 1H), 1.31 (d, J=6.4 Hz, 3H), 1.18 (d, J=6.8 Hz, 3H), 1.07 (t, J=7.2 Hz, 3H) ppm. MS: M/e 505 (M+1)⁺.

### Compound A220: 2-(7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-d]pyrimidin-2-yl)acetonitrile, or 2-(7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-d]pyrimidin-2-yl)acetonitrile

### Step A: 7-((2S,5R)-2,5-dimethyl-4-((S)-1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one, or 7-((2S,5R)-2,5-dimethyl-4-((R)-1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one

To a solution of Intermediate 9 (266 mg, 1 mmol, optical isomer) in CH₃CN (5 mL) was added DIPEA (258 mg, 2 mmol), followed by POCl₃ (307 mg, 2 mmol) and one drop of DMF. The reaction was stirred at 80° C. for 5 h. The reaction was cooled to room temperature, diluted with sat NaHCO₃ solution, extracted with EA (60 mL×3), washed with brine, dried over Na₂SO₄, filtered, and concentrated to dryness. The resulting residue was dissolved in CH₃CN (4 mL), Intermediate 1a (162 mg, 0.6 mmol, optical isomer) and DIPEA (258 mg, 2 mmol) were added. The resulting mixture was heated at 80° C. for 15 h. The reaction was quenched with water, extracted with EA (60 mL×2), washed with brine, dried over Na₂SO₄, filtered, and concentrated to dryness. The resulting residue was purified by Prep-TLC (DCM:MeOH=15:1) to give titled compound (75 mg, 14%). MS: M/e 503 (M+1)⁺.

### Step B: 7-((2S,5R)-2,5-dimethyl-4-((S)-1-(quinoxalin-6-yl)ethyl)piperazin-1-yl)-4-methyl-2,4-dihydro-5H-pyrazolo[4,3-d]pyrimidin-5-one, or 7-((2S,5R)-2,5-dimethyl-4-((R)-1-(quinoxalin-6-yl)ethyl)piperazin-1-yl)-4-methyl-2,4-dihydro-5H-pyrazolo[4,3-d]pyrimidin-5-one

To a solution of 7-((2S,5R)-2,5-dimethyl-4-((S)-1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one, or 7-((2S,5R)-2,5-dimethyl-4-((R)-1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one (57 mg, 0.149 mmol) in DCM (1 mL) was added TFA (4 mL). The resulting mixture was stirred at room temperature for 16 hours. The reaction mixture was concentrated under reduced pressure, diluted with water/DCM, basified with saturated NaHCO₃ solution to pH 7-8, extracted with DCM:IPA (4:1, 60 mL×2). The combined organic layers were washed with brine, dried over Na₂SO₄, filtered and concentrated. The resulting residue was purified by flash column chromatography (DCM:MeOH=10:1) to give titled compound (31 mg, 50%). MS: M/e 419 (M+1)⁺.

### Step C: 2-(7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-d]pyrimidin-2-yl)acetonitrile, or 2-(7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-d]pyrimidin-2-yl)acetonitrile

To a solution of 7-((2S,5R)-2,5-dimethyl-4-((S)-1-(quinoxalin-6-yl)ethyl)piperazin-1-yl)-4-methyl-2,4-dihydro-5H-pyrazolo[4,3-d]pyrimidin-5-one, or 7-((2S,5R)-2,5-dimethyl-4-((R)-1-(quinoxalin-6-yl)ethyl)piperazin-1-yl)-4-methyl-2,4-dihydro-5H-pyrazolo[4,3-d]pyrimidin-5-one (31 mg, 0.074 mmol) and K₂CO₃ (14 mg, 0.148 mmol) in DMF (2 mL) was added 2-iodoacetonitrile (19 mg, 0.111 mmol). The reaction was stirred at room temperature overnight. The reaction mixture was diluted with water, extracted with EA (60 mL×2), washed with brine, dried over Na₂SO₄, filtered, and concentrated to dryness. The resulting residue was purified by Prep-TLC (DCM:MeOH=15:1) and Prep-HPLC(Method A) to give the titled compound (27 mg, 79%) as a single diastereoisomer. ¹HNMR (400 MHz, CD₃OD) δ 8.91-8.85 (m, 2H), 8.16-8.02 (m, 3H), 7.97-7.86 (m, 1H), 6.00-5.85 (m, 0.5H), 5.56 (s, 1H), 5.45 (s, 1H), 5.40-5.28 (m, 1H), 4.77-4.65 (m, 0.5H), 4.02-3.89 (m, 1H), 3.76-3.64 (m, 0.5H), 3.40 (s, 3H), 3.36-3.31 (m, 0.5H), 3.09-2.89 (m, 3H), 1.64-1.48 (m, 3H), 1.44 (d, J=6.8 Hz, 3H), 0.97 (d, J=6.8 Hz, 3H) ppm. MS: M/e 458 (M+1)⁺.

### Compound A224: 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluorobenzonitrile

### Step A: tert-butyl (2S,5R)-4-(1-(2-cyano-4-fluorophenyl)ethyl)-2,5-diethylpiperazine-1-carboxylate

A sealed tube was charged with tert-butyl (2S,5R)-4-(1-(2-bromo-4-fluorophenyl)ethyl)-2,5-diethylpiperazine-1-carboxylate (220 mg, 0.5 mmol), zinc cyanide (88 mg, 0.75 mmol), Pd(PPh₃)₄ (60 mg, 0.05 mmol) and DMF (5 ml). The mixture was stirred at 120° C. overnight, cooled to RT Water was added and the aqueous was extracted with EA. The combined organic layers were washed with brine, dried over Na₂SO₄, and concentrated to dryness. The resulting residue was purified by flash column chromatography (EA:PE=0-100% in 25 minutes) to give the product 300 mg (crude). MS: M/e 390 (M+1)⁺.

### Step B: 2-(1-((2R,5S)-2,5-diethylpiperazin-1-yl)ethyl)-5-fluorobenzonitrile

To a solution of tert-butyl (2S,5R)-4-(1-(2-cyano-4-fluorophenyl)ethyl)-2,5-diethylpiperazine-1-carboxylate (100 mg, crude) in DCM (4 mL) was added TFA (1.5 ml). The mixture was stirred at RT for 2 hours. The mixture was concentrated to dryness and diluted with water. The mixture was extracted with EA. The aqueous phase was adjusted pH to 12-13 with saturated sodium carbonate solution and extracted with DCM. The combined organic layers were washed with brine, dried over Na₂SO₄, and concentrated to dryness. The resulting residue (40 mg, 54%.) was used in the next step without further purification. MS: M/e 290 (M+1)⁺.

### Step C: 2-(1-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)ethyl)-5-fluorobenzonitrile

To a solution of 2-(1-((2R,5S)-2,5-diethylpiperazin-1-yl)ethyl)-5-fluorobenzonitrile (40 mg, 0.14 mmol) in dioxane (2 mL) was added Intermediate 2 (103 mg, 0.27 mmol) and DIPEA (54 mg, 0.42 mmol). The resulting mixture was stirred at 100° C. overnight. The reaction was quenched with water, extracted with EA, washed with brine, dried over Na₂SO₄, filtered, and concentrated to dryness. The resulting residue was purified by flash column chromatography (MeOH/DCM) to give the product (50 mg, 69%). MS: M/e 521 (M+1)⁺.

### Step D: 2-(1-((2R,5S)-2,5-diethyl-4-(4-methyl-5-oxo-4,5-dihydro-2H-pyrazolo[4,3-b]pyridin-7-yl)piperazin-1-yl)ethyl)-5-fluorobenzonitrile

To a solution of 2-(1-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)ethyl)-5-fluorobenzonitrile (50 mg) in MeOH (3 mL) was added HCl (1 ml, 4 M in 1,4-dioxane). The reaction was stirred at room temperature for 2 hours. The solvent was removed under reduced pressure. The resulting residue (25 mg, crude) was used directly to next step without further purification. MS: M/e 437 (M+1)⁺.

### Step E: 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluorobenzonitrile

To a solution of 2-(1-((2R,5S)-2,5-diethyl-4-(4-methyl-5-oxo-4,5-dihydro-2H-pyrazolo[4,3-b]pyridin-7-yl)piperazin-1-yl)ethyl)-5-fluorobenzonitrile (25 mg, crude) in DMF (2 mL) was added potassium carbonate (24 mg, 0.17 mmol) and 2-iodoacetonitrile (14 mg, 0.1 mmol). The resulting mixture was stirred at room temperature overnight. The reaction mixture was diluted with water, extracted with EA, washed with brine, dried over Na₂SO₄, filtered, and concentrated to dryness, to obtain Compound A224. The resulting residue containing Compound A224 was purified to give Compound A224a (1 mg, 3.7%) and Compound A224b (1 mg, 3.7%) by Prep-HPLC (Method A).

Compound A224a (the earlier peak): ¹H NMR (400 MHz, CD₃OD) δ 7.92 (s, 1H), 7.71 (dd, J=8.8, 5.5 Hz, 1H), 7.55 (dd, J=8.4, 2.6 Hz, 1H), 7.45 (td, J=8.5, 2.7 Hz, 1H), 5.57 (s, 1H), 5.46 (s, 2H), 4.19 (d, J=6.4 Hz, 1H), 3.43 (s, 3H), 3.36 (s, 2H), 2.99 (d, J=12.4 Hz, 1H), 2.90 (d, J=8.8 Hz, 1H), 2.79 (s, 1H), 2.40 (s, 1H), 2.11 (s, 1H), 1.86-1.73 (m, 1H), 1.69-1.54 (m, 2H), 1.42 (d, J=6.5 Hz, 3H), 0.90 (s, 3H), 0.78 (t, J=7.2 Hz, 3H) ppm. MS: M/e 476 (M+1)⁺.

Compound A224b (the later peak): ¹H NMR (400 MHz, CD₃OD) δ 7.93 (s, 1H), 7.75 (dd, J=8.7, 5.5 Hz, 1H), 7.53 (dd, J=8.4, 2.6 Hz, 1H), 7.44 (td, J=8.5, 2.7 Hz, 1H), 5.56 (s, 1H), 5.47 (s, 2H), 3.97 (q, J=6.4 Hz, 1H), 3.52 (d, J=11.9 Hz, 1H), 3.43 (s, 3H), 3.22-3.11 (m, 1H), 2.76 (d, J=8.8 Hz, 2H), 2.26-2.18 (m, 1H), 2.10-1.85 (m, 2H), 1.80-1.64 (m, 2H), 1.52 (dd, J=13.8, 7.5 Hz, 1H), 1.40 (d, J=6.7 Hz, 3H), 1.04 (t, J=7.3 Hz, 3H), 0.61 (t, J=7.4 Hz, 3H) ppm. MS: M/e 476 (M+1)⁺.

### Compound A225: 2-(7-((2R,5R)-5-ethyl-2-(methoxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: methyl (R)-2-((S)-2-(((benzyloxy)carbonyl)amino)-3-hydroxypropanamido)butanoate

A mixture of methyl (R)-2-aminobutanoate hydrochloride (7.2 g, 30 mmol), ((benzyloxy)carbonyl)-L-serine (5 g, 33 mmol), HATU (13.8 g, 36 mmol) and DIPEA (7.7 g, 60 mmol) in CH₂Cl₂ (100 mL) was stirred overnight. The reaction mixture was Washed with H₂O, aq. citric acid, brine, dried over Na₂SO₄ and concentrated to give the titled compound (9 g, 88.7%). MS: M/e 339 (M+1)⁺.

### Step B: (3R,6S)-3-ethyl-6-(hydroxymethyl)piperazine-2,5-dione

To a stirred solution of methyl (R)-2-((S)-2-(((benzyloxy)carbonyl)amino)-3-hydroxypropanamido)butanoate (9 g, 26.6 mmol) in MeOH (80 mL) was added Pd/C (1 g, 10% in water). After the addition, the reaction mixture was stirred over a weekend under H₂ (1 atm). The reaction mixture was filtered. The filtrate was stirred at 100° C. for 2 days in a sealed tube. The reaction mixture was allowed cool to room temperature and filtered. The cake was collected, dried to give the titled compound (2.8 g, 61%). MS: M/e 173 (M+1)⁺.

### Step C: ((2R,5R)-5-ethylpiperazin-2-yl)methanol

A mixture of (3R,6S)-3-ethyl-6-(hydroxymethyl)piperazine-2,5-dione (2.8 g, 16.3 mmol) in BH₃-THF (1.0 M, 50 mL) was stirred at 70° C. overnight. The reaction mixture was quenched with MeOH at 0° C. The reaction mixture was concentrated to give the residue, which was directly used to the next step. MS: M/e 145 (M+1)⁺.

### Step D: tert-butyl (2R,5R)-2-ethyl-5-(hydroxymethyl)piperazine-1-carboxylate

To a stirred solution of ((2R,5R)-5-ethylpiperazin-2-yl)methanol (crude, 16.3 mmol) in MeOH (50 mL) was added Et₃N (6.4 g, 64 mmol), followed by Boc₂O (10.6 g, 48.8 mmol). After the addition, the reaction mixture was stirred at 60° C. over a weekend. The reaction mixture was concentrated to give the residue, treated with EA (100 mL), washed with H₂O, brine, dried over Na₂SO₄, concentrated to give the intermediate di-tert-butyl (2R,5R)-2-ethyl-5-(hydroxymethyl)piperazine-1,4-dicarboxylate (0.81 g) as colorless oil, which was dissolved in EtOH (20 mL), and aq. NaOH (0.8 g, in 5 mL H₂O) was added. Then the mixture was stirred at 95° C. overnight. The reaction mixture was acidified to pH=10-11 with aq. citric acid, extracted with EA (10 mL×4). The combined organic layers were washed with brine, dried over Na₂SO₄ and concentrated to dryness. The resulting residue was purified by flash column chromatography to give the titled compound (230 mg, 6%), which was solidified after standing. MS: M/e 245 (M+1)⁺.

### Step E: tert-butyl (2R,5R)-2-ethyl-5-(hydroxymethyl)-4-(4-methyl-5-oxo-2-(tetrahydro-2H-pyran-2-yl)-4,5-dihydro-2H-pyrazolo[4,3-b]pyridin-7-yl)piperazine-1-carboxylate

A mixture of Intermediate 2 (326 mg, 0.85 mmol), tert-butyl (2R,5R)-2-ethyl-5-(hydroxymethyl)piperazine-1-carboxylate (230 mg, 0.94 mmol) and DIEPA (220 mg, 1.7 mmol) in dioxane (10 mL) was stirred for 2 days in a sealed tube at 120° C. The reaction mixture was diluted with EA (15 mL), washed with H₂O, brine, dried over Na₂SO₄ and concentrated to dryness. The resulting residue was purified by flash column chromatography to give the titled compound (210 mg, 52%). MS: M/e 476 (M+1)⁺.

### Step F: tert-butyl (2R,5R)-2-ethyl-5-(methoxymethyl)-4-(4-methyl-5-oxo-2-(tetrahydro-2H-pyran-2-yl)-4,5-dihydro-2H-pyrazolo[4,3-b]pyridin-7-yl)piperazine-1-carboxylate

To a stirred suspension of NaH (60%, 24 mg, 0.6 mmol) in THF (5 mL) was added a solution of tert-butyl (2R,5R)-2-ethyl-5-(hydroxymethyl)-4-(4-methyl-5-oxo-2-(tetrahydro-2H-pyran-2-yl)-4,5-dihydro-2H-pyrazolo[4,3-b]pyridin-7-yl)piperazine-1-carboxylate (95 mg, 0.2 mmol) in THF (2 mL). After stirred for 30 min, Mel (85.2 mg, 0.6 mmol) was added. Then the reaction was stirred at RT overnight. The reaction mixture was quenched with aq·NH₄Cl, extracted with EA (10 mL×2). The combined organic layers were washed with brine, dried over Na₂SO₄, concentrated to give the residue, which was used to the next step directly. MS: M/e 490 (M+1)⁺.

### Step G: 7-((2R,5R)-5-ethyl-2-(methoxymethyl)-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a stirred solution of tert-butyl (2R,5R)-2-ethyl-5-(methoxymethyl)-4-(4-methyl-5-oxo-2-(tetrahydro-2H-pyran-2-yl)-4,5-dihydro-2H-pyrazolo[4,3-b]pyridin-7-yl)piperazine-1-carboxylate (crude, 0.2 mmol) in CH₂Cl₂ (5 mL) was added TFA (2 mL). After stirred for 3 hours, the reaction mixture was concentrated to give the residue, basified to pH=10-12 with aq·Na₂CO₃, extracted with CH₂Cl₂: IPA (3: 1, 15 mL×3). The combined organic layers were washed with brine, dried over Na₂SO₄, concentrated to give the intermediate (60 mg, 0.154 mmol), which was dissolved in CH₃CN (3 mL), 1-(quinoxalin-6-yl)ethan-1-ol (80 mg, 0.462 mmol), (cyanomethyl)trimethylphosphonium iodide (112 mg, 0.462 mmol) and DIPEA (200 mg, 1.54 mmol) were added. After then, the reaction mixture was stirred at 100° C. overnight. The reaction mixture was diluted with EA (15 mL), washed with brine, dried over Na₂SO₄ and concentrated to dryness. The resulting residue was purified by Prep-TLC (CH₂Cl₂: MeOH=10:1) to give the titled compound (15 mg, 14%). MS: M/e 546 (M+1)⁺.

### Step H: 2-(7-((2R,5R)-5-ethyl-2-(methoxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a stirred solution of 7-((2R,5R)-5-ethyl-2-(methoxymethyl)-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (15 mg, 0.028 mmol) in MeOH (5 mL) was added HCl (4 M in 1,4-dioxane, 2 mL). After the addition, the reaction mixture was stirred overnight. The reaction was concentrated to give the residue, which was dissolved in DMF/H₂O (3 mL/0.5 mL), and K₂CO₃ (15.18 mg, 0.11 mmol) was added, followed by 2-iodoacetonitrile (2 drops). Then the mixture was stirred for 2 days. The reaction mixture was poured into H₂O (20 mL), extracted with EA (15 mL×3). The combined organic layers were washed with brine, dried over Na₂SO₄ and concentrated to dryness. The resulting residue was purified by Pre-TLC (CH₂Cl₂: MeOH=10:1) to give the titled compound (4 mg). ¹H NMR (400 MHz, CD₃OD) δ 8.90-8.85 (m, 2H), 8.15-7.90 (m, 4H), 5.63 (d, J=4.8 Hz, 1H), 5.46 (s, 2H), 4.14-3.99 (m, 1H), 3.98-3.88 (m, 1H), 3.81-3.68 (m, 1H), 3.63-3.47 (m, 2H), 3.44 (s, 3H), 3.36 (s, 1H), 3.28-3.19 (m, 2H), 3.11 (s, 2H), 3.04-2.92 (m, 0.5H), 2.81-2.73 (m, 0.5H), 2.53-2.42 (m, 1H), 1.74-1.59 (m, 2H), 1.44 (t, J=7.2 Hz, 3H), 1.05 (t, J=7.3 Hz, 2H), 0.68 (t, J=7.4 Hz, 1H) ppm. MS: M/e 501 (M+1)⁺.

### Compound A229: 2-(7-((2R,5R)-5-ethyl-2-(hydroxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: 7-((2R,5R)-2-(((tert-butyldimethylsilyl)oxy)methyl)-5-ethylpiperazin-1-yl)-4-methyl-2-(tetrahydro-2H-pyran-2-yl)-2,4-dihydro-5H-pyrazolo[4,3-b]pyridin-5-one

To a stirred solution of tert-butyl (2R,5R)-2-ethyl-5-(hydroxymethyl)-4-(4-methyl-5-oxo-2-(tetrahydro-2H-pyran-2-yl)-4,5-dihydro-2H-pyrazolo[4,3-b]pyridin-7-yl)piperazine-1-carboxylate (115 mg, 0.24 mmol) in CH₂Cl₂ (5 mL) was added TFA (1 mL). After then, the reaction mixture was stirred for 2 hours. The reaction mixture was concentrated to give the residue, basified to pH=10-12, extracted with CH₂Cl₂:IPA (3:1, 15 mL×3). The combined organic layers were washed with brine, dried over Na₂SO₄, concentrated to give the residue, which was dissolved in CH₂Cl₂ (5 mL), Et₃N (48.5 mg, 0.48 mmol) was added, then DMAP (3 mg, 0.024 mmol), followed by TBSCl (54.7 mg, 0.363 mmol). After the addition, the reaction mixture was stirred at RT overnight. The reaction mixture was concentrated and the resulting residue was purified by Pre-TLC (CH₂Cl₂: MeOH=10:1) to give the titled compound (30 mg, 26%). MS: M/e 490 (M+1)⁺.

### Step B: 7-((2R,5R)-2-(((tert-butyldimethylsilyl)oxy)methyl)-5-ethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of 7-((2R,5R)-2-(((tert-butyldimethylsilyl)oxy)methyl)-5-ethylpiperazin-1-yl)-4-methyl-2-(tetrahydro-2H-pyran-2-yl)-2,4-dihydro-5H-pyrazolo[4,3-b]pyridin-5-one (30 mg, 0.0613 mmol), 1-(quinoxalin-6-yl)ethan-1-ol (32 mg, 0.18 mmol), (cyanomethyl)trimethylphosphonium iodide (44 mg, 0.18 mmol) and DIPEA (79 mg, 0.613 mmol) in CH₃CN (3 mL) was stirred at 100° C. overnight. The reaction mixture was diluted with EA (15 mL), washed with H₂O, brine, dried over Na₂SO₄ and concentrated to dryness. The resulting residue was purified by Prep-TLC (CH₂Cl₂: MeOH=10:1) to give the titled compound (20 mg, 51%). MS: M/e 646 (M+1)⁺.

### Step C: 2-(7-((2R,5R)-5-ethyl-2-(hydroxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a stirred solution of 7-((2R,5R)-2-(((tert-butyldimethylsilyl)oxy)methyl)-5-ethyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (20 mg, 0.03 mmol) in MeOH (3 mL) was added HCl (2 mL, 4 M in 1,4-dioxane). Then the reaction mixture was stirred overnight. The reaction mixture was concentrated to give the residue, which was dissolved in DMF/H₂O (3 mL/0.5 mL) and K₂CO₃ (16.56 mg, 0.12 mmol) was added, followed by 2-iodoacetonitrile (2 drops). Then stirred overnight. The reaction mixture was poured into H₂O (15 mL) and extracted with EA (10 mL×3). The combined organic layers were washed with brine, dried over Na₂SO₄ and concentrated to dryness. The resulting residue was purified by Prep-TLC (CH₂Cl₂: MeOH=10:1) to give the titled compound (5 mg). ¹H NMR (400 MHz, CD₃OD) δ ppm 8.91-8.84 (m, 2H), 8.14-7.98 (m, 3H), 7.93 (s, 1H), 5.65 (d, J=7.2 Hz, 1H), 5.46 (d, J=3.2 Hz, 2H), 4.19-4.03 (m, 1H), 3.96-3.73 (m, 2H), 3.63-3.55 (m, 1H), 3.44 (s, 3H), 3.27-3.19 (m, 2H), 3.01-2.74 (m, 2H), 2.59-2.37 (m, 1H), 1.79-1.57 (m, 2H), 1.52-1.40 (m, 3H), 1.04 (t, J=7.2 Hz, 1.5H), 0.68 (t, J=7.2 Hz, 1.5H) ppm. MS: M/e 487 (M+1)⁺.

### Compound A232: 2-(7-((2S,5R)-2,5-diethyl-4-(1-(1-methyl-3-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile

### Step A: 1-(1-methyl-3-(trifluoromethyl)-1H-pyrazol-4-yl)ethan-1-ol

To a solution of 1-methyl-3-(trifluoromethyl)-1H-pyrazole-4-carbaldehyde (200 mg, 1.12 mmol) in THF (8 ml) at −60° C. under N₂, was added MeMgBr (3M, 0.41 ml, 1.23 mmol) dropwise. The solution was warmed to RT naturally and stirred at RT overnight. After completed, the solution was quenched with H₂O (10 ml) and then extracted with EA (10 ml×2). The organic layer was washed with aq. NaHCO₃ (10 ml), brine (10 ml), dried over anhydrous Na₂SO₄ and then concentrated under reduced pressure to give the titled compound (217 mg, 100%), which was used to the next step directly without further purification. MS: M/e 195 (M+1)⁺.

### Step B: 2-(7-((2S,5R)-2,5-diethyl-4-(1-(1-methyl-3-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile

A mixture of Intermediate 5 (50 mg, 0.15 mmol), 1-(1-methyl-3-(trifluoromethyl)-1H-pyrazol-4-yl)ethan-1-ol (44 mg, 0.23 mmol), (cyanomethyl)trimethylphosphonium iodide (111 mg, 0.46 mmol) and DIPEA (197 mg, 1.53 mmol) in MeCN (2 ml) was stirred at 100° C. overnight. After completed, the solution was diluted with EA (15 ml), washed with brine (10 ml), dried and concentrated under reduced pressure. The resulting residue was purified by Prep-TLC with DCM:MeOH (20:1) and Prep-HPLC to give the titled compound (12 mg). ¹H NMR (400 MHz, DMSO-d6) δ 7.94 (d, J=2.7 Hz, 1H), 7.90-7.80 (m, 1H), 5.58 (s, 2H), 5.34 (s, 1H), 3.87 (s, 0.5H), 3.85 (d, J=4.3 Hz, 3H), 3.67 (d, J=5.9 Hz, 0.5H), 3.27 (s, 2.5H), 3.23 (s, 3H), 3.15 (s, 0.5H), 2.91 (d, J=11.1 Hz, 0.5H), 2.74 (s, 1H), 2.51 (d, J=18.8 Hz, 1H), 2.37 (d, J=11.5 Hz, 0.5H), 1.90-1.73 (m, 1H), 1.60-1.26 (m, 3H), 1.22 (t, J=8.0 Hz, 3H), 0.89-0.75 (m, 3H), 0.69-0.56 (m, 3H) ppm. MS: M/e 505 (M+1)⁺.

### Compound A233: 2-(7-((2S,5R)-2,5-diethyl-4-(1-(1-methyl-5-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile

### Step A: 1-(1-methyl-5-(trifluoromethyl)-1H-pyrazol-4-yl)ethan-1-ol

To a solution of 1-methyl-5-(trifluoromethyl)-1H-pyrazole-4-carbaldehyde (200 mg, 1.12 mmol) in THF (8 ml) at −60° C. under N₂, was added MeMgBr (3M, 0.41 ml, 1.23 mmol) dropwise. The solution was warmed to RT naturally and stirred at RT overnight. After completed, the solution was quenched with H₂O (10 ml) and then extracted with EA (10 ml×2). The organic layer was washed with aq. NaHCO₃ (10 ml), brine (10 ml), dried over anhydrous Na₂SO₄ and then concentrated under reduced pressure to give the titled compound (217 mg, 100%), which was used directly for the next step without further purification. MS: M/e 195 (M+1)⁺.

### Step B: 2-(7-((2S,5R)-2,5-diethyl-4-(1-(1-methyl-5-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile

A mixture of Intermediate 5 (50 mg, 0.15 mmol), 1-(1-methyl-5-(trifluoromethyl)-1H-pyrazol-4-yl)ethan-1-ol (44 mg, 0.23 mmol), (cyanomethyl)trimethylphosphonium iodide (111 mg, 0.46 mmol) and DIPEA (197 mg, 1.53 mmol) in MeCN (2 ml) was stirred at 100° C. overnight. After completed, the solution was diluted with EA (15 ml), washed with brine (10 ml), dried and concentrated under reduced pressure. The resulting residue was purified by Prep-TLC with DCM:MeOH (20:1) and Prep-HPLC to give the titled compound (13.6 mg). ¹H NMR (400 MHz, DMSO-d6) δ 7.94 (d, J=3.5 Hz, 1H), 7.59 (d, J=24.7 Hz, 1H), 5.58 (s, 2H), 5.34 (d, J=8.4 Hz, 1H), 3.99-3.93 (m, 0.5H), 3.91 (s, 3H), 3.73 (q, J=5.6 Hz, 0.5H), 3.27 (s, 2H), 3.23 (s, 3H), 3.12 (d, J=14 Hz, 0.5H), 2.93 (d, J=9.0 Hz, 0.5H), 2.81-2.61 (m, 1H), 2.56-2.48 (m, 1H), 2.39-2.27 (m, 1H), 1.87-1.72 (m, 1H), 1.58-1.29 (m, 3H), 1.24 (dd, J=19.6, 6.3 Hz, 3H), 0.90-0.73 (m, 3H), 0.69-0.56 (m, 3H) ppm. MS: M/e 505 (M+1)⁺.

### Compound A236: 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-2H-pyrazolo[4,3-b]pyridin-2-yl)acetonitrile

### Step A: 7-((2S,5R)-4-(1-(4-fluoro-2-(trifluoromethyl)phenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

A mixture of Intermediate 10B (100 mg, 0.29 mmol), 1-(4-fluoro-2-(trifluoromethyl)phenyl)ethan-1-ol (230 mg, 1.1 mmol), (cyanomethyl)trimethylphosphonium iodide (290 mg, 1.2 mmol) and DIPEA (400 mg, 3.1 mmol) in MeCN (2 mL) was stirred at 100° C. for 24 hours. The resulting mixture was diluted with EA (10 mL), washed with brine (5 mL×3), dried and concentrated to dryness. The resulting residue was purified by flash column chromatography to give the titled compound (35 mg, 22%). MS: M/e 536 (M+1)⁺.

### Step B: 7-((2S,5R)-4-(1-(4-fluoro-2-(trifluoromethyl)phenyl)ethyl)-2,5-dimethylpiperazin-1-yl)-4-methyl-2,4-dihydro-5H-pyrazolo[4,3-b]pyridin-5-one

To a solution of 7-((2S,5R)-4-(1-(4-fluoro-2-(trifluoromethyl)phenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (25 mg, 0.047 mmol) in MeOH (2 mL) was added HCl (1 mL, 4 M in 1,4-dioxane) at room temperature and the resulting mixture was stirred at room temperature for 16 hours. The mixture was concentrated, basified with NH₃ (2 mL, 7M in MeOH). The mixture was concentrated to dryness. The resulting residue was treated with DCM (10 mL), filtered and the filtrate was concentrated to dryness to give the titled compound (15 mg, 70%). MS: M/e 452 (M+1)⁺.

### Step C: 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-2H-pyrazolo[4,3-b]pyridin-2-yl)acetonitrile

To a solution of 7-((2S,5R)-4-(1-(4-fluoro-2-(trifluoromethyl)phenyl)ethyl)-2,5-dimethylpiperazin-1-yl)-4-methyl-2,4-dihydro-5H-pyrazolo[4,3-b]pyridin-5-one (15 mg, 0.033 mmol), K₂CO₃ (25 mg, 0.18 mmol) and H₂O (2 drops) in DMF (1 mL) was added 2-iodoacetonitrile (20 mg, 0.12 mmol). The resulting mixture was stirred at room temperature for 3 days. The reaction mixture was diluted with EA (10 mL), washed with brine (3 mL×5), dried over Na₂SO₄, and concentrated to dryness. The resulting residue was purified by flash column chromatography to obtain Compound A236, and further separated into Compound A236a (1.4 mg) and Compound A236b (0.9 mg) by Prep-HPLC (Method A).

Compound A236a (the earlier peak): ¹H NMR (400 MHz, CD₃OD) δ 8.12-7.99 (m, 1H), 7.93 (s, 1H), 7.48-7.30 (m, 2H), 5.56 (s, 1H), 5.47 (s, 2H), 4.00-3.87 (m, 1H), 3.82-3.46 (m, 3H), 3.43 (s, 3H), 2.88-2.68 (m, 2H), 2.01 (d, J=12.0 Hz, 1H), 1.33 (d, J=6.8 Hz, 3H), 1.22-1.10 (m, 6H) ppm. MS: M/e 491 (M+1)⁺.

Compound A236b (the later peak): ¹H NMR (400 MHz, CD₃OD) δ 8.15-8.05 (m, 1H), 7.93 (s, 1H), 7.48-7.33 (m, 2H), 5.56 (s, 1H), 5.47 (s, 2H), 4.16-4.02 (m, 1H), 3.50-3.37 (m, 5H), 3.09-3.00 (m, 1H), 2.94 (d, J=11.6 Hz, 1H), 2.87-2.76 (m, 2H), 1.45 (d, J=6.4 Hz, 3H), 1.27 (d, J=6.4 Hz, 3H), 1.01 (d, J=6.8 Hz, 3H) ppm. MS: M/e 491 (M+1)⁺.

Compound A238: 2-(but-2-yn-1-yl)-7-((2S,5R)-2,5-diethyl-4-(1-(4-fluoro-2-(trifluoromethyl)phenyl)ethyl)piperazin-1-yl)-4-methyl-2,4-dihydro-5H-pyrazolo[4,3-b]pyridin-5-one

A mixture of 7-((2S,5R)-2,5-diethyl-4-(1-(4-fluoro-2-(trifluoromethyl)phenyl)ethyl)piperazin-1-yl)-4-methyl-2,4-dihydro-5H-pyrazolo[4,3-b]pyridin-5-one (40 mg, 0.083 mmol), 1-bromobut-2-yne (23 mg, 0.17 mmol), K₂CO₃ (34 mg, 0.25 mmol) and H₂O (100 mg, 5.5 mmol) in DMF (1 mL) was stirred at room temperature for 2 hours. The mixture was diluted with EA (10 mL), washed with brine (5 mL×3), dried over Na₂SO₄, and concentrated to dryness. The resulting residue was purified by flash column chromatography to obtain Compound A238, and further purified by Prep-HPLC (Method A) to give Compound A238a (1.64 mg) and Compound A238b (2.16 mg).

Compound A238a (the earlier peak). ¹H NMR (400 MHz, CD₃OD) δ 8.04 (dd, J=9.6, 5.6 Hz, 1H), 7.90 (s, 1H), 7.47-7.32 (m, 2H), 5.60-5.41 (m, 1H), 5.14-4.93 (m, 2H), 4.08-3.96 (m, 1H), 3.54-3.39 (m, 4H), 3.21-3.13 (m, 1H), 2.73-2.62 (m, 1H), 2.15 (d, J=12.4 Hz, 1H), 2.05-1.78 (m, 4H), 1.78-1.37 (m, 4H), 1.36-1.22 (m, 4H), 1.15-0.95 (m, 3H), 0.70-0.45 (m, 3H) ppm. MS: M/e 532 (M+1)⁺.

Compound A238b (the later peak). ¹H NMR (400 MHz, CD₃OD) δ 8.13-8.02 (m, 1H), 7.89 (s, 1H), 7.49-7.34 (m, 2H), 5.53 (s, 1H), 5.03 (s, 2H), 4.25-4.11 (m, 1H), 3.44 (s, 3H), 3.13-3.04 (m, 1H), 2.97-2.64 (m, 2H), 2.38-2.09 (m, 2H), 2.08-1.76 (m, 4H), 1.72-1.34 (m, 3H), 1.33-1.17 (m, 4H), 1.10-0.84 (m, 3H), 0.79-0.51 (m, 3H) ppm. MS: M/e 532 (M+1)⁺.

### Compound A240: 3-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-1-ethyl-1H-pyrazole-4-carbonitrile

### Step A: 4-bromo-N-methoxy-N-methyl-1H-pyrazole-5-carboxamide

To a solution of 4-bromo-1H-pyrazole-5-carboxylic acid (2 g, 10.5 mmol) in DCM (100 mL) and was added N,O-dimethylhydroxylamine hydrochloride (5 g, 52.3 mmol), HATU (6 g, 15.7 mmol) and DIPEA (6.7 g, 52.3 mmol). The resulting mixture was stirred at RT overnight. The reaction solvent was removed under reduce pressure. The resulting residue was purified by flash column chromatography (DCM/MeOH) to give the titled compound (0.9 g, 37%). MS: M/e 234 (M+1)⁺.

### Step B: 4-bromo-1-ethyl-N-methoxy-N-methyl-1H-pyrazole-3-carboxamide

To a solution of 4-bromo-N-methoxy-N-methyl-1H-pyrazole-5-carboxamide (0.9 g, 3.86 mmol) in DMF (15 mL) and was added iodoethane (1.8 g, 11.6 mmol), K₂CO₃ (1.6 g, 11.6 mmol). The reaction was stirred at 40° C. overnight. The reaction mixture was poured into water and extracted with EA. The organic layer was removed under reduce pressure. The resulting residue was purified by flash column chromatography (EA/PE) to give 4-bromo-1-ethyl-N-methoxy-N-methyl-1H-pyrazole-3-carboxamide (540 mg) and 4-bromo-1-ethyl-N-methoxy-N-methyl-1H-pyrazole-5-carboxamide (210 mg). MS: M/e 262 (M+1)⁺.

### Step C: 1-(4-bromo-1-ethyl-1H-pyrazol-3-yl)ethan-1-one

To a solution of 4-bromo-1-ethyl-N-methoxy-N-methyl-1H-pyrazole-3-carboxamide (0.3 g, 1.15 mmol) in THF (10 mL) was added CH₃MgBr (0.76 ml, 2.3 mmol, 3M) at 0° C. The resulting mixture was stirred for 30 minutes at RT. The reaction mixture was quenched with saturated NH₄Cl aq. and extracted with EA. The organic layer was removed under reduce pressure. The resulting residue was purified by flash column chromatography (DCM/MeOH) to give the titled compound (200 mg, 81%). MS: M/e 217 (M+1)⁺

### Step D: 1-(4-bromo-1-ethyl-1H-pyrazol-3-yl)ethan-1-ol

To a solution of 1-(4-bromo-1-ethyl-1H-pyrazol-3-yl)ethan-1-one (0.2 g, 0.92 mmol) in MeOH (5 mL) and was added NaBH₄ (52 mg, 1.38 mmol) at 0° C. The resulting mixture was stirred for 30 minutes at RT.

The reaction mixture was quenched with water and extracted with DCM. The organic layer was dried and concentrated to give the titled compound (180 mg, 89%). MS: M/e 201 (M−17)⁺

### Step E: 7-((2S,5R)-4-(1-(4-bromo-1-ethyl-1H-pyrazol-3-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one

To a solution of 1-(4-bromo-1-ethyl-1H-pyrazol-3-yl)ethan-1-ol (59 mg, 0.268 mmol) in CH₃CN (5 mL) and was added Intermediate 3 (50 mg, 0.134 mmol), (cyanomethyl)trimethylphosphonium iodide (130 mg, 0.536 mmol) and DIPEA (173 mg, 1.34 mmol). The resulting mixture was stirred at 105° C. overnight. The reaction solvent was concentrated. The resulting residue was purified by Prep-TLC (DCM:MeOH=20:1) to give the titled compound (40 mg, 52%). MS: M/e 574 (M+1)⁺.

### Step F: 3-(1-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)ethyl)-1-ethyl-1H-pyrazole-4-carbonitrile

To a solution of 7-((2S,5R)-4-(1-(4-bromo-1-ethyl-1H-pyrazol-3-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one (15 mg, 0.026 mmol) in THF (3 mL)/water (0.8 mL) and was added tBuXPhos Pd G3 (5 mg). The resulting mixture was stirred at 50° C. overnight. The reaction solvent was concentrated. The resulting residue was purified by Prep-TLC (DCM:MeOH=20:1) to give the titled compound (15 mg, crude). MS: M/e 521 (M+1)⁺.

### Step G: 3-(1-((2R,5S)-2,5-diethyl-4-(4-methyl-5-oxo-4,5-dihydro-2H-pyrazolo[4,3-b]pyridin-7-yl)piperazin-1-yl)ethyl)-1-ethyl-1H-pyrazole-4-carbonitrile

To a solution of 3-(1-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)ethyl)-1-ethyl-1H-pyrazole-4-carbonitrile (15 mg, 0.029 mmol) in DCM (1 mL) was added TFA (2 mL). The resulting mixture was stirred at RT for 3 hours. The reaction solvent was removed under vacuum. The crude product was dissolved in water and the aqueous was adjusted to pH=9 with sat. NaHCO₃ aq. The aqueous layer was extracted with (DCM:i-PrOH=4:1). The organic layer was dried and concentrated. The resulting residue was purified by Prep-TLC (DCM:MeOH=15:1) to give the titled compound (8 mg, 63%). MS: M/e 437 (M+1)⁺.

### Step H: 3-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-1-ethyl-1H-pyrazole-4-carbonitrile

To a solution of 3-(1-((2R,5S)-2,5-diethyl-4-(4-methyl-5-oxo-4,5-dihydro-2H-pyrazolo[4,3-b]pyridin-7-yl)piperazin-1-yl)ethyl)-1-ethyl-1H-pyrazole-4-carbonitrile (8 mg, 0.018 mmol) in DMF (2 mL) were added K₂CO₃ (26 mg, 0.189 mmol) and 2-iodoacetonitrile (6 mg, 0.037 mmol). The resulting mixture was stirred at RT overnight. The reaction mixture was quenched by saturated NaCl aq. and extracted with EA. The organic layer was washed with brine, dried over Na₂SO₄ and concentrated to dryness. The resulting residue was was purified by Prep-TLC (DCM:MeOH=15:1) and purified by Prep-HPLC (Method A) to give the titled compound (1.04 mg). ¹H NMR (400 MHz, CD₃OD) δ 8.24 (s, 1H), 7.93 (s, 1H), 5.56 (s, 1H), 5.48 (s, 2H), 4.25-4.12 (m, 2H), 4.10-3.75 (m, 1H), 3.55-3.34 (m, 4H), 3.10-2.60 (m, 4H), 2.55-2.25 (m, 1H), 2.12-1.97 (m, 1H), 1.80-1.51 (m, 3H), 1.49-1.35 (m, 6H), 1.05-0.80 (m, 3H), 0.79-0.60 (m, 3H) ppm. MS: M/e 476 (M+1)⁺.

Compound A243 and A244: 2-(7-((2S,5R)-2,5-dimethyl-4-((S)-1-(quinoxalin-6-yl)ethyl)piperazin-1-yl)-4-methyl-5-oxo-3-(trifluoromethyl)-4,5-dihydro-2H-pyrazolo[4,3-b]pyridin-2-yl)acetonitrile or 2-(7-((2S,5R)-2,5-dimethyl-4-((R)-1-(quinoxalin-6-yl)ethyl)piperazin-1-yl)-4-methyl-5-oxo-3-(trifluoromethyl)-4,5-dihydro-2H-pyrazolo[4,3-b]pyridin-2-yl)acetonitrile; and 2-(7-((2S,5R)-2,5-dimethyl-4-((S)-1-(quinoxalin-6-yl)ethyl)piperazin-1-yl)-4-methyl-5-oxo-3-(trifluoromethyl)-4,5-dihydro-1H-pyrazolo[4,3-b]pyridin-1-yl)acetonitrile or 2-(7-((2S,5R)-2,5-dimethyl-4-((R)-1-(quinoxalin-6-yl)ethyl)piperazin-1-yl)-4-methyl-5-oxo-3-(trifluoromethyl)-4,5-dihydro-1H-pyrazolo[4,3-b]pyridin-1-yl)acetonitrile Compound A243 (15 mg, 43%) and Compound A244 (6 mg, 17%) were prepared according to the similar procedures as described for Compound A209 using Intermediate 1a under appropriate conditions that could be recognized by one skilled in the art. The single positional isomers were separated by Prep-HPLC (Method A).

Compound A243 (the earlier isomer): ¹HNMR (400 MHz, CD₃OD) δ 8.91-8.84 (m, 2H), 8.15-8.01 (m, 3H), 5.67 (s, 3H), 4.87-4.79 (m, 1H), 4.35-4.12 (m, 1H), 4.04-3.91 (m, 1H), 3.57-3.42 (m, 4H), 3.16-3.03 (m, 1H), 2.98-2.86 (m, 2H), 1.52-1.38 (m, 6H), 1.07 (d, J=6.8 Hz, 3H) ppm. MS: M/e 525 (M+1)⁺.

Compound A244 (the later isomer): ¹HNMR (400 MHz, CD₃OD) δ 8.94-8.86 (m, 2H), 8.22-7.94 (m, 3H), 6.57-6.32 (m, 1H), 5.86-5.62 (m, 1.3H), 5.49-5.25 (m, 0.7H), 5.14-5.00 (m, 0.3H), 4.75-4.57 (m, 0.7H), 3.65 (s, 3H), 3.55-3.31 (m, 2H), 3.25-3.10 (m, 1H), −2.82-2.45 (m, 2H), 2.26-2.05 (m, 1H), 1.82-1.55 (m, 3H), 1.27-0.99 (m, 6H) ppm. MS: M/e 525 (M+1)⁺.

Compound A245: 2-(3-chloro-7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-d]pyrimidin-2-yl)acetonitrile, or 2-(3-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-d]pyrimidin-2-yl)acetonitrile

### Step A: 3-chloro-4-methyl-2-(tetrahydro-2H-pyran-2-yl)-2,4-dihydro-5H-pyrazolo[4,3-d]pyrimidine-5,7 (6H)-dione

To a solution of Intermediate 9 (270 mg, 1.1 mmol) in DMF (10 mL) was added NCS (150 mg, 1.1 mmol). The reaction was stirred at 40° C. for 5 h. The reaction was cooled to room temperature, diluted with water, extracted with DCM:IPA (4:1, 60 mL×4) and concentrated to dryness. The resulting residue was purified by flash column chromatography (DCM:MeOH=15:1) to give titled compound (320 mg, crude). ¹HNMR (400 MHz, DMSO-d₆) δ 11.38 (s, 1H), 5.63 (dd, J=2.8 Hz, 9.6 Hz, 1H), 3.89-3.79 (m, 1H), 3.69-3.57 (m, 1H), 3.43 (s, 3H), 2.30-2.15 (m, 1H), 2.05-1.94 (m, 1H), 1.93-1.83 (m, 1H), 1.76-1.60 (m, 1H), 1.57-1.45 (m, 2H) ppm MS: M/e 285 (M+1)⁺.

### Step B: 3-chloro-7-((2S,5R)-2,5-dimethyl-4-((R)-1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one, or 3-chloro-7-((2S,5R)-2,5-dimethyl-4-((S)-1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one

To a solution of 3-chloro-4-methyl-2-(tetrahydro-2H-pyran-2-yl)-2,4-dihydro-5H-pyrazolo[4,3-d]pyrimidine-5,7 (6H)-dione (230 mg, 0.8 mmol) in CH₃CN (5 mL) was added DIPEA (208 mg, 1.6 mmol), followed by POCl₃ (245 mg, 1.6 mmol) and a drop of DMF. The reaction was stirred at 80° C. for 6 h. The reaction was cooled to room temperature, diluted with sat NaHCO₃ solution, extracted with EA (50 mL×3), washed with brine, dried over Na₂SO₄, filtered, and concentrated to dryness. The resulting residue was dissolved in CH₃CN (4 mL), Intermediate 1a (217 mg, 0.6 mmol, optical isomer) and DIPEA (208 mg, 1.6 mmol) were added. The resulting mixture was heated at 80° C. for 15 hours. The reaction mixture was quenched with water, extracted with EA (60 mL×3). The combined organic layers were washed with brine, dried over Na₂SO₄, filtered, and concentrated to dryness. The resulting residue was purified by Prep-TLC (DCM:MeOH=15:1) to give the titled compound (40 mg, 9%). MS: M/e 537 (M+1)⁺.

### Step C: 3-chloro-7-((2S,5R)-2,5-dimethyl-4-((R)-1-(quinoxalin-6-yl)ethyl)piperazin-1-yl)-4-methyl-2,4-dihydro-5H-pyrazolo[4,3-d]pyrimidin-5-one, or 3-chloro-7-((2S,5R)-2,5-dimethyl-4-((S)-1-(quinoxalin-6-yl)ethyl)piperazin-1-yl)-4-methyl-2,4-dihydro-5H-pyrazolo[4,3-d]pyrimidin-5-one

To a solution of 3-chloro-7-((2S,5R)-2,5-dimethyl-4-((R)-1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one, or 3-chloro-7-((2S,5R)-2,5-dimethyl-4-((S)-1-(quinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one (40 mg, 0.0746 mmol) in DCM (1 mL) was added TFA (3 mL). The resulting mixture was stirred at room temperature for 16 hours. The mixture was concentrated under reduced pressure, diluted with water/DCM, basified with saturated NaHCO₃ aq. to pH 7-8, extracted with DCM (40 mL×2). The combined organic layers were washed with brine, dried over Na₂SO₄, filtered, concentrated. The resulting residue was purified by flash column chromatography (DCM:MeOH=10:1) to give titled compound (20 mg, 60%). MS: M/e 453 (M+1)⁺.

### Step D: 2-(3-chloro-7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-d]pyrimidin-2-yl)acetonitrile, or 2-(3-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-d]pyrimidin-2-yl)acetonitrile

To a solution of 3-chloro-7-((2S,5R)-2,5-dimethyl-4-((R)-1-(quinoxalin-6-yl)ethyl)piperazin-1-yl)-4-methyl-2,4-dihydro-5H-pyrazolo[4,3-d]pyrimidin-5-one, or 3-chloro-7-((2S,5R)-2,5-dimethyl-4-((S)-1-(quinoxalin-6-yl)ethyl)piperazin-1-yl)-4-methyl-2,4-dihydro-5H-pyrazolo[4,3-d]pyrimidin-5-one (20 mg, 0.044 mmol) and K₂CO₃ (12 mg, 0.088 mmol) in DMF (2 mL) was added 2-chloroacetonitrile (5 mg, 0.066 mmol). The reaction was stirred at room temperature overnight. The reaction mixture was diluted with water, extracted with EA (60 mL×2), washed with brine, dried over Na₂SO₄, filtered, and concentrated to dryness.

The resulting residue was purified by Prep-TLC (DCM:MeOH=15:1) and Prep-HPLC to give the titled compound (10 mg, 50%). ¹HNMR (400 MHz, CD₃OD) δ 8.91-8.85 (m, 2H), 8.16-8.01 (m, 3H), 5.92-5.76 (m, 0.5H), 5.65-5.45 (m, 2H), 5.39-5.23 (m, 1H), 4.78-4.58 (m, 0.5H), 4.02-3.91 (m, 1H), 3.76-3.67 (m, 0.5H), 3.64 (s, 3H), 3.38-3.32 (m, 0.5H), 3.10-2.88 (m, 3H), 1.66-1.47 (m, 3H), 1.36 (d, J=6.4 Hz, 3H), 0.99 (d, J=6.4 Hz, 3H) ppm. MS: M/e 492 (M+1)⁺.

### Compound A247: 2-(7-((2S,5R)-4-(1-(2-(difluoromethoxy)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile

### Step A: 7-((2S,5R)-4-(1-(2-(difluoromethoxy)-4-fluorophenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

A mixture of the product of Intermediate 10B (69 mg, 0.2 mmol), 1-(2-(difluoromethoxy)-4-fluorophenyl)ethan-1-ol (82.4 mg, 0.4 mmol), (cyanomethyl)trimethylphosphonium iodide (146 mg, 0.6 mmol) and DIPEA (258 mg, 2 mmol) in CH₃CN (4 mL) was stirred at 100° C. for 2 days in a sealed tube. The reaction mixture was diluted with EA (15 mL), washed with H₂O, brine, dried over Na₂SO₄ and concentrated to dryness. The resulting residue was purified by Pre-TLC (CH₂Cl₂:MeOH=10:1) to give the titled compound (36 mg, 34%). MS: M/e 534 (M+1)⁺.

### Step B: 2-(7-((2S,5R)-4-(1-(2-(difluoromethoxy)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile

To a stirred solution of 7-((2S,5R)-4-(1-(2-(difluoromethoxy)-4-fluorophenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (36 mg, 0.067 mmol) in MeOH (3 mL) was added HCl (2 mL, 4 M in 1,4-dioxane). Then the reaction mixture was stirred overnight. The reaction mixture was concentrated to give the residue, which was dissolved in DMF/H₂O (3 mL/0.5 mL) and K₂CO₃ (28 mg, 0.201 mmol) was added, followed by 2-iodoacetonitrile (22.55 mg, 0.134 mmol). Then stirred overnight. The reaction mixture was poured into H₂O (15 mL) and extracted with EA (15 mL×2). The combined organic layers were washed with brine, dried over Na₂SO₄ and concentrated to dryness. The resulting residue was purified by Pre-TLC (CH₂Cl₂:MeOH=10:1) to give the titled compound (5 mg). ¹H NMR (400 MHz, CD₃OD) δ 8.77 (s, 1H), 8.50-8.39 (m, 1H), 8.27-8.03 (m, 1H), 7.96-7.85 (m, 2H), 6.43-6.38 (m, 2H), 6.17 (d, J=9.6 Hz, 1H), 5.31 (s, 0.5H), 4.82-4.58 (m, 1H), 4.30-4.16 (m, 2H), 4.04 (s, 3H), 4.02-3.96 (m, 0.5H), 3.70-3.38 (m, 2H), 2.80 (d, J=11.2 Hz, 1H), 2.10-1.95 (m, 5H), 1.90-1.78 (m, 3H), 1.68 (d, J=6.4 Hz, 1H) ppm. MS: M/e 489 (M+1)⁺.

### Compound A254: 2-(7-((2S,5R)-4-(1-(2-(1,1-difluoroethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile

### Step A: 1-bromo-2-(1,1-difluoroethyl)-4-fluorobenzene

To a solution of BAST (4.4 g, 20 mmol) and MeOH (2 drops) was added 1-(2-bromo-5-fluorophenyl)ethan-1-one (2.2 g, 10 mmol). After the addition, the reaction mixture was stirred at 70° C. overnight. The reaction mixture was added dropwise to NaHCO₃ aq., extracted with EA (20 mL×2). The combined organic layers were washed with brine, dried over Na₂SO₄ and concentrated to dryness. The resulting residue was purified by flash column chromatography to give the titled compound (1.6 g, 67%). ¹H NMR (400 MHz, CDCl₃) δ 7.61 (dd, J=8.8, 5.2 Hz, 1H), 7.35 (dd, J=9.2, 3.2 Hz, 1H), 7.00 (td, J=8.4, 3.2 Hz, 1H), 2.05 (t, J=18.4 Hz, 3H) ppm.

### Step B: 2-(1,1-difluoroethyl)-4-fluorobenzaldehyde

To a stirred solution of 1-bromo-2-(1,1-difluoroethyl)-4-fluorobenzene (1.6 g, 6.69 mmol) in THF (20 mL) was added dropwise n-BuLi (1.6 M, 4.6 mL, 7.36 mmol) below −80° C. Then DMF (602 mg, 8.03 mmol) was added dropwise. The reaction mixture was quenched with NH₄Cl aq. at −80° C. and extracted with EA (20 mL×2). The combined organic layers were washed with brine, dried over Na₂SO₄ and concentrated to dryness. The resulting residue was purified by flash column chromatography to give the titled compound (450 mg, 36%). ¹H NMR (400 MHz, CDCl₃) δ 10.41 (s, 1H), 8.11 (dd, J=8.4, 5.6 Hz, 1H), 7.33 (dd, J=9.2, 2.4 Hz, 1H), 7.29-7.23 (m, 1H), 2.09 (t, J=18.4 Hz, 3H) ppm.

### Step C: 1-(2-(1,1-difluoroethyl)-4-fluorophenyl)ethan-1-ol

To a stirred solution of 2-(1,1-difluoroethyl)-4-fluorobenzaldehyde (188 mg, 1 mmol) in THF (10 mL) was added dropwise MeMgBr (3.0 M, 0.37 mL, 1.1 mmol) at 0° C. After the addition, the reaction was quenched with NH₄Cl aq. and extracted with EA (10 mL×2). The combined organic layers were washed with brine, dried over Na₂SO₄ and concentrated to dryness. The resulting residue was purified by flash column chromatography to give the titled compound (132 mg, 65%). ¹H NMR (400 MHz, CDCl₃) δ 7.73 (dd, J=9.6, 5.6 Hz, 1H), 7.20-7.13 (m, 2H), 5.34 (q, J=5.6 Hz, 1H), 1.98 (t, J=18.4 Hz, 3H), 1.49 (d, J=6.4 Hz, 3H) ppm.

### Step D: 7-((2S,5R)-4-(1-(2-(1,1-difluoroethyl)-4-fluorophenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

A mixture of the product of Intermediate 10B (69 mg, 0.2 mmol), 1-(2-(1,1-difluoroethyl)-4-fluorophenyl)ethan-1-ol (81.6 mg, 0.4 mmol), (cyanomethyl)trimethylphosphonium iodide (146 mg, 0.6 mmol) and DIPEA (258 mg, 2 mmol) in CH₃CN (4 mL) was stirred at 100° C. overnight in a sealed tube. The reaction mixture was diluted with EA (15 mL), washed with H₂O, brine, dried over Na₂SO₄ and concentrated to dryness. The resulting residue was purified by Pre-TLC (EA) to give the titled compound (38 mg, 36%). MS: M/e 532 (M+1)⁺.

### Step E: 2-(7-((2S,5R)-4-(1-(2-(1,1-difluoroethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile

To a stirred solution of 7-((2S,5R)-4-(1-(2-(1,1-difluoroethyl)-4-fluorophenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (38 mg, 0.07 mmol) in MeOH (3 mL) was added HCl (2 mL, 4 M in 1,4-dioxane). Then the reaction mixture was stirred overnight. The reaction mixture was concentrated to give the residue, which was dissolved in DMF/H₂O (3 mL/0.5 mL) and K₂CO₃ (20 mg, 0.14 mmol) was added, followed by 2-iodoacetonitrile (23.1 mg, 0.14 mmol). Then the reaction mixture was stirred overnight. The reaction mixture was poured into H₂O (15 mL) and extracted with EA (10 mL×2). The combined organic layers were washed with brine, dried over Na₂SO₄ and concentrated to dryness. The resulting residue was purified by Pre-TLC (CH₂Cl₂:MeOH=10:1) to give the titled compound (5 mg). ¹H NMR (400 MHz, CD₃OD) δ 8.07-7.95 (m, 1H), 7.93 (s, 1H), 7.28-7.17 (m, 2H), 5.56 (s, 1H), 5.47 (s, 2H), 4.27-3.99 (m, 1H), 3.79-3.58 (m, 1.5H), 3.43 (s, 3H), 3.42-3.36 (m, 0.5H), 3.08-2.80 (m, 3H), 2.11-1.92 (m, 4H), 1.47-0.99 (m, 9H) ppm. MS: M/e 487 (M+1)⁺.

### Compound A257: 2-(7-((2S,5R)-4-(1-(4-fluoro-2-(trifluoromethyl)phenyl)ethyl)-2,5-dimethylpiperazin-1-yl)-4-(methyl-d3)-5-oxo-4,5-dihydro-2H-pyrazolo[4,3-b]pyridin-2-yl)acetonitrile

### Step A: methyl 4-(N-(methyl-d3)acetamido)-1-(tetrahydro-2H-pyran-2-yl)-1H-pyrazole-3-carboxylate

To a solution of methyl 4-acetamido-1-(tetrahydro-2H-pyran-2-yl)-1H-pyrazole-3-carboxylate (2.67 g, 10 mmol) in DMF (15 mL) was added Cs₂CO₃ (9.78 g, 30 mmol), followed by CD₃I (4.35 g, 30 mmol). The reaction was stirred at RT for 16 hours. The reaction mixture was diluted with water, extracted with EA (80 mL×3). The combined organic layers were washed with brine, dried over Na₂SO₄, filtered, and concentrated to dryness. The resulting residue was purified by flash column chromatography (EA/PE) to give titled compound (2.47 g, 86%). MS: M/e 285 (M+1)⁺.

### Step B: 7-hydroxy-4-(methyl-d3)-2-(tetrahydro-2H-pyran-2-yl)-2,4-dihydro-5H-pyrazolo[4,3-b]pyridin-5-one

To a solution of methyl 4-(N-(methyl-d3)acetamido)-1-(tetrahydro-2H-pyran-2-yl)-1H-pyrazole-3-carboxylate (2.47 g, 8.67 mmol) in THF (40 mL) was added drop w is a solution of L1HMDS (˜14 mL, 1 mol/L, ˜1.6 eq) at −70° C. The reaction was stirred for 1 h, quenched with water, warmed to RT slowly, extracted with EA (80 mL). The water layer was collected, treated with citric acid to pH 3-4, extracted with DCM:IPA (3:1.60 mL×3), dried over Na₂SO₄, filtered, and concentrated to dryness. The resulting residue was purified by flash column chromatography (DCM:MeOH=15:1) to give titled compound (1.47 g, 66%) MS: M/e 253 (M+1)⁺.

### Step C: 4-(methyl-d3)-5-oxo-2-(tetrahydro-2H-pyran-2-yl)-4,5-dihydro-2H-pyrazolo[4,3-b]pyridin-7-yl trifluoromethanesulfonate

To a solution of 7-hydroxy-4-(methyl-d₃)-2-(tetrahydro-2H-pyran-2-yl)-2,4-dihydro-5H-pyrazolo[4,3-b]pyridin-5-one (1.47 g, 5.8 mmol) in THF/DMF (20 mL/10 mL) was added K₂CO₃ (1.6 g, 11.6 mmol), followed by 1,1,1-trifluoro-N-phenyl-N-((trifluoromethyl)sulfonyl)methanesulfonamide (2.07 g, 5.8 mmol). The reaction was stirred at room temperature for 36 h. The mixture was diluted with water, extracted with EA (60 mL×2), washed with brine, dried over Na₂SO₄, filtered, and concentrated to dryness. The resulting residue was purified by flash column chromatography (EA:PE=1:1) to give titled compound (0.87 g, 39%). ¹HNMR (400 MHz, CDCl₃) δ 7.65 (s, 1H), 6.58 (s, 1H), 5.58 (dd, J=2.8 Hz, 8.4 Hz, 1H), 4.10-4.00 (m, 1H), 3.82-3.70 (m, 1H), 2.26-1.96 (m, 3H), 1.82-1.63 (m, 3H) ppm. MS: M/e 385 (M+1)⁺.

### Step D: 7-((2S,5R)-4-(1-(4-fluoro-2-(trifluoromethyl)phenyl)ethyl)-2,5-dimethylpiperazin-1-yl)-4-(methyl-d3)-2-(tetrahydro-2H-pyran-2-yl)-2,4-dihydro-5H-pyrazolo[4,3-b]pyridin-5-one

To a solution of 4-(methyl-d3)-5-oxo-2-(tetrahydro-2H-pyran-2-yl)-4,5-dihydro-2H-pyrazolo[4,3-b]pyridin-7-yl trifluoromethanesulfonate (115 mg, 0.3 mmol) and (2R,5S)-1-(1-(4-fluoro-2-(trifluoromethyl)phenyl)ethyl)-2,5-dimethylpiperazine (100 mg, 0.33 mmol) in CH₃CN (3 mL) was added DIPEA (78 mg, 0.62 mmol). Then the mixture was heated at 90° C. under N₂ for 60 h. The mixture was cooled to room temperature, diluted with water (50 mL), extracted with EA (60 mL×3). The combined organic layers were washed with brine, dried over Na₂SO₄, filtered and concentrated to dryness. The resulting residue was purified by flash column chromatography (DCM:MeOH=15:1) to give titled compound (90 mg, 56%). MS: M/e 539 (M+1)⁺.

### Step E: 7-((2S,5R)-4-(1-(4-fluoro-2-(trifluoromethyl)phenyl)ethyl)-2,5-dimethylpiperazin-1-yl)-4-(methyl-d3)-2,4-dihydro-5H-pyrazolo[4,3-b]pyridin-5-one

To a solution of 7-((2S,5R)-4-(1-(4-fluoro-2-(trifluoromethyl)phenyl)ethyl)-2,5-dimethylpiperazin-1-yl)-4-(methyl-d3)-2-(tetrahydro-2H-pyran-2-yl)-2,4-dihydro-5H-pyrazolo[4,3-b]pyridin-5-one (90 mg, 0.167 mmol) in DCM (1 mL) was added TFA (6 mL). The resulting mixture was stirred at room temperature overnight. Another portion of TFA (10 mL) was added and the reaction was stirred for 6 hours. The reaction mixture was concentrated under reduced pressure, diluted with water/DCM, basified with saturated NaHCO₃ aq. to pH 7-8, extracted with DCM:IPA (4:1, 60 mL×2). The combined organic layers were washed with brine, dried over Na₂SO₄, filtered and concentrated to dryness. The resulting residue was purified by Prep-TLC (DCM:MeOH=10:1) to give titled compound (45 mg, 67%). MS: M/e 455 (M+1)⁺.

### Step F: 2-(7-((2S,5R)-4-(1-(4-fluoro-2-(trifluoromethyl)phenyl)ethyl)-2,5-dimethylpiperazin-1-yl)-4-(methyl-d3)-5-oxo-4,5-dihydro-2H-pyrazolo[4,3-b]pyridin-2-yl)acetonitrile

To a solution of 7-((2S,5R)-4-(1-(4-fluoro-2-(trifluoromethyl)phenyl)ethyl)-2,5-dimethylpiperazin-1-yl)-4-(methyl-d3)-2,4-dihydro-5H-pyrazolo[4,3-b]pyridin-5-one (45 mg, 0.1 mmol) and K₂CO₃ (69 mg, 0.5 mmol) in DMF (3 mL) was added 2-chloroacetonitrile (22 mg, 0.3 mmol). The reaction was stirred at room temperature overnight. The reaction mixture was diluted with water, extracted with EA (60 mL×2). The combined organic layers were washed with brine, dried over Na₂SO₄, filtered, and concentrated to dryness.

The resulting residue was purified by Prep-TLC (DCM:MeOH=15:1) to give the titled compound (24 mg, 48%). ¹HNMR (400 MHz, CD₃OD) δ 8.15-8.02 (m, 1H), 7.93 (s, 1H), 7.46-7.35 (m, 2H), 5.56 (s, 1H), 5.48 (s, 2H), 4.15-4.05 (m, 0.5H), 3.96-3.88 (m, 0.5H), 3.71-3.59 (m, 1H), 3.44-3.36 (m, 0.5H), −3.28 (m, 2H), 3.07-2.90 (m, 1H), 2.86-2.76 (m, 1H), 2.04-1.97 (m, 0.5H), 1.44 (d, J=6.0 Hz, 1.5H), 1.33 (d, J=6.4 Hz, 1.5H), 1.26 (d, J=6.0 Hz, 1.5H), 1.17 (t, J=7.6 Hz, 3H), 1.01 (d, J=6.0 Hz, 1.5H) ppm. MS: M/e 494 (M+1)⁺.

Compound A258: 2-(7-((2S,5R)-2,5-dimethyl-4-((R)-1-(quinoxalin-6-yl)ethyl)piperazin-1-yl)-3-fluoro-4-methyl-5-oxo-4,5-dihydro-1H-pyrazolo[4,3-b]pyridin-1-yl)acetonitrile, or 2-(7-((2S,5R)-2,5-dimethyl-4-((S)-1-(quinoxalin-6-yl)ethyl)piperazin-1-yl)-3-fluoro-4-methyl-5-oxo-4,5-dihydro-1H-pyrazolo[4,3-b]pyridin-1-yl)acetonitrile

### Step A: methyl 4-acetamido-5-fluoro-1H-pyrazole-3-carboxylate

To a solution of methyl 4-acetamido-1-(tetrahydro-2H-pyran-2-yl)-1H-pyrazole-3-carboxylate (4 g, 15 mmol) in CH₃CN (150 mL) was added Selectfluor (10.35 g, 30 mmol). The reaction was stirred at 40° C. for 18 hours. The reaction mixture was quenched with water and extracted with DCM (160 mL×2). The combined organic layers were washed with water, brine, dried over Na₂SO₄, filtered and concentrated to dryness. The resulting residue was purified by flash column chromatography to give the titled compound (0.24 g, 8%). ¹HNMR (400 MHz, CDCl₃) δ 7.40 (s, 1H), 3.96 (s, 3H), 2.21 (s, 3H) ppm. MS: M/e 202 (M+1)⁺.

### Step B: Methyl 4-acetamido-5-fluoro-1-(tetrahydro-2H-pyran-2-yl)-1H-pyrazole-3-carboxylate

To a solution of methyl 4-acetamido-5-fluoro-1H-pyrazole-3-carboxylate (830 mg, 4.13 mmol) and TsOH·H₂O (76 mg, 0.4 mmol) in THF (10 mL) was added 3,4-Dihydro-2H-pyran (693 mg, 8.26 mmol). The reaction was stirred at room temperature for 18 hours. Then the reaction mixture was concentrated to dryness. The resulting residue was purified by flash column chromatography (EA/PE) to give the titled compound (400 mg, 34%). MS: M/e 286 (M+1)⁺.

### Step C: methyl 5-fluoro-4-(N-methylacetamido)-1-(tetrahydro-2H-pyran-2-yl)-1H-pyrazole-3-carboxylate

To a solution of methyl 4-acetamido-5-fluoro-1-(tetrahydro-2H-pyran-2-yl)-1H-pyrazole-3-carboxylate (610 mg, 2.14 mmol) in DMF (10 mL) was added Cs₂CO₃ (2.09 g, 6.42 mmol), followed by CH₃I (0.91 g, 6.42 mmol). The reaction was stirred at RT for 16 hours. The reaction mixture was diluted with water, extracted with DCM (60 mL×2), washed with brine, dried over Na₂SO₄, filtered, and concentrated to dryness. The resulting residue was purified by flash column chromatography (EA:PE=1:1) to give the titled compound (0.58 g, 82%). ¹HNMR (400 MHz, CDCl₃) δ 6.22-6.14 (m, 1H), 4.09-3.98 (m, 1H), 3.93 (s, 3H), 3.76-3.64 (m, 1H), 3.17-3.10 (m, 3H), 2.45-2.28 (m, 1H), 2.16-2.07 (m, 1H), 2.01-1.84 (m, 4H), 1.78-1.63 (m, 3H) ppm. MS: M/e 300 (M+1)⁺.

### Step D: 3-fluoro-7-hydroxy-4-methyl-2-(tetrahydro-2H-pyran-2-yl)-2,4-dihydro-5H-pyrazolo[4,3-b]pyridin-5-one

To a solution of methyl 5-fluoro-4-(N-methylacetamido)-1-(tetrahydro-2H-pyran-2-yl)-1H-pyrazole-3-carboxylate (0.58 g, 1.93 mmol) in THF (10 mL) was added drop wis a solution of L1HMDS (3.1 mL, 1 mol/L) at −70° C. The reaction was stirred at −70° C. for 2 hours, quenched with water, extracted with EA (80 mL). The water layer was collected, treated with citric acid to PH 3-4, extracted with DCM:IPA (4:1, 60 mL×2). The combined organic layers were dried over Na₂SO₄, filtered, and concentrated to dryness. The resulting residue was subjected to slurry with EA/PE to give the titled compound (95 mg, 18%) ¹HNMR (400 MHz, DMSO-d₆) δ 11.98 (s, 1H), 5.86 (d, J=10 Hz, 1H), 5.76 (s, 1H), 3.94-3.84 (m, 1H), 3.61-3.50 (m, 1H), 3.42 (s, 3H), 2.25-2.10 (m, 1H), 2.02-1.93 (m, 1H), 1.92-1.82 (m, 1H), 1.72-1.57 (m, 1H), 1.54-1.44 (m, 2H) ppm. MS: M/e 268 (M+1)⁺.

### Step E: 3-fluoro-4-methyl-5-oxo-2-(tetrahydro-2H-pyran-2-yl)-4,5-dihydro-2H-pyrazolo[4,3-b]pyridin-7-yl trifluoromethanesulfonate

To a solution of 3-fluoro-7-hydroxy-4-methyl-2-(tetrahydro-2H-pyran-2-yl)-2,4-dihydro-5H-pyrazolo[4,3-b]pyridin-5-one (95 mg, 0.355 mmol) in DCM (6 mL) was added DMAP (1 mg) and TEA (107 mg, 1.06 mmol) at 0° C. Then a solution of Tf₂O (100 mg, 0.355 mmol) in DCM (1 mL) was added dropwise. The reaction was stirred at 0° C. for 15 minutes. The reaction mixture was diluted with water, extracted with DCM (40 mL×3), washed with brine, dried over Na₂SO₄, filtered, and concentrated to give the titled compound, which was used to next step directly without further purification. MS: M/e 400 (M+1)⁺.

### Step F: 7-((2S,5R)-2,5-dimethyl-4-((R)-1-(quinoxalin-6-yl)ethyl)piperazin-1-yl)-3-fluoro-4-methyl-2-(tetrahydro-2H-pyran-2-yl)-2,4-dihydro-5H-pyrazolo[4,3-b]pyridin-5-one, or 7-((2S,5R)-2,5-dimethyl-4-((S)-1-(quinoxalin-6-yl)ethyl)piperazin-1-yl)-3-fluoro-4-methyl-2-(tetrahydro-2H-pyran-2-yl)-2,4-dihydro-5H-pyrazolo[4,3-b]pyridin-5-one

To a solution of 3-fluoro-4-methyl-5-oxo-2-(tetrahydro-2H-pyran-2-yl)-4,5-dihydro-2H-pyrazolo[4,3-b]pyridin-7-yl trifluoromethanesulfonate (crude, 0.355 mmol) and 6-(1-((2R,5S)-2,5-dimethylpiperazin-1-yl)ethyl)quinoxaline (intermediate 1a) (95 mg, 0.355 mmol, optical isomer) in CH₃CN (4 mL) was added DIPEA (130 mg, 1.06 mmol). Then the mixture was heated at 90° C. under N₂ for 18 h. The mixture was cooled to room temperature, diluted with water (50 mL), extracted with EA (60 mL×2), washed with brine, dried over Na₂SO₄, filtered and concentrated to dryness. The resulting residue was purified by flash column chromatography (DCM:MeOH=15:1) to give the titled compound (8 mg, 4% for two steps). MS: M/e 520 (M+1)⁺.

### Step G: 7-((2S,5R)-2,5-dimethyl-4-((R)-1-(quinoxalin-6-yl)ethyl)piperazin-1-yl)-3-fluoro-4-methyl-2,4-dihydro-5H-pyrazolo[4,3-b]pyridin-5-one, or 7-((2S,5R)-2,5-dimethyl-4-((S)-1-(quinoxalin-6-yl)ethyl)piperazin-1-yl)-3-fluoro-4-methyl-2,4-dihydro-5H-pyrazolo[4,3-b]pyridin-5-one

To a solution of 7-((2S,5R)-2,5-dimethyl-4-((R)-1-(quinoxalin-6-yl)ethyl)piperazin-1-yl)-3-fluoro-4-methyl-2-(tetrahydro-2H-pyran-2-yl)-2,4-dihydro-5H-pyrazolo[4,3-b]pyridin-5-one, or 7-((2S,5R)-2,5-dimethyl-4-((S)-1-(quinoxalin-6-yl)ethyl)piperazin-1-yl)-3-fluoro-4-methyl-2-(tetrahydro-2H-pyran-2-yl)-2,4-dihydro-5H-pyrazolo[4,3-b]pyridin-5-one (8 mg, 0.0154 mmol) in DCM (1 mL) was added TFA (5 mL). The resulting mixture was stirred at room temperature overnight. The mixture was concentrated to dryness under reduced pressure to give the titled compound (8 mg, crude). MS: M/e 436 (M+1)⁺.

### Step H: 2-(7-((2S,5R)-2,5-dimethyl-4-((R)-1-(quinoxalin-6-yl)ethyl)piperazin-1-yl)-3-fluoro-4-methyl-5-oxo-4,5-dihydro-1H-pyrazolo[4,3-b]pyridin-1-yl)acetonitrile, or 2-(7-((2S,5R)-2,5-dimethyl-4-((S)-1-(quinoxalin-6-yl)ethyl)piperazin-1-yl)-3-fluoro-4-methyl-5-oxo-4,5-dihydro-1H-pyrazolo[4,3-b]pyridin-1-yl)acetonitrile

To a solution of 7-((2S,5R)-2,5-dimethyl-4-((R)-1-(quinoxalin-6-yl)ethyl)piperazin-1-yl)-3-fluoro-4-methyl-2,4-dihydro-5H-pyrazolo[4,3-b]pyridin-5-one, or 7-((2S,5R)-2,5-dimethyl-4-((S)-1-(quinoxalin-6-yl)ethyl)piperazin-1-yl)-3-fluoro-4-methyl-2,4-dihydro-5H-pyrazolo[4,3-b]pyridin-5-one (8 mg, crude) and K₂CO₃ (25 mg, 0.154 mmol) in DMF (2 mL) was added 2-chloroacetonitrile (3 mg, 0.03 mmol). The reaction was stirred at room temperature for 5 hours. The reaction mixture was diluted with water, extracted with EA (60 mL), washed with brine, dried over Na₂SO₄, filtered, and concentrated to dryness. The resulting residue was purified by Prep-TLC (DCM:MEOH=15:1) to give the titled compound (4.5 mg, 61% for two steps). ¹HNMR (400 MHz, CD₃OD) δ 8.92-8.87 (m, 2H), 8.16-7.96 (m, 3H), 6.35-6.18 (m, 1H), 5.49-5.41 (m, 1H), 5.24-5.12 (m, 1H), 4.65-4.35 (m, 1H), 3.61 (s, 3H), 3.56-3.42 (m, 1H), 3.40-3.31 (m, 2H), 2.90-2.65 (m, 2H), 2.60-2.50 (m, 1H), 1.75-1.53 (m, 3H), 1.25-1.19 (m, 3H), 1.17-1.08 (m, 3H) ppm. MS: M/e 475 (M+1)⁺.

Compound A259: 2-(7-((2R,5R)-5-ethyl-2-((R)-1-hydroxyethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: methyl (R)-2-((2S,3R)-2-(((benzyloxy)carbonyl)amino)-3-hydroxybutanamido)butanoate

To a solution of ((benzyloxy)carbonyl)-L-threonine (15.0 g, 0.06 mol), methyl (R)-2-aminobutanoate (6.9 g, 0.06 mol) and DIPEA (15.5 g, 0.12 mol) in DCM (300 mL) was added HATU (34.3 g, 0.09 mol) at 0° C. The reaction mixture was stirred at room temperature overnight. The reaction mixture was quenched by water and washed with water (500 mL×2), HCl (200 mL, 1 N) and brine (500 mL×2). The organic layers were concentrated under reduced pressure to give the titled compound (19 g, 89%). MS: M/e 353 (M+1)⁺.

### Step B: (3R,6S)-3-ethyl-6-((R)-1-hydroxyethyl)piperazine-2,5-dione

To a solution of (3R,6S)-3-ethyl-6-((R)-1-hydroxyethyl)piperazine-2,5-dione (19 g, 0.05 mol) in MeOH (100 mL) was added Pd/C (1.9 g, 10% in water) at room temperature. The resulting mixture was stirred at room temperature under H₂ (50 psi) atmosphere for overnight. The reaction mixture was cooled to r.t and filtered. The filtrates were heated to 100° C. in a sealed tube overnight. The reaction mixture was cooled to RT, filtered and concentrated to give the titled compound (5.9 g, 59%). MS: M/e 187 (M+1)⁺.

### Step C: (R)-1-((2R,5R)-5-ethylpiperazin-2-yl)ethan-1-ol

A mixture of (3R,6S)-3-ethyl-6-((R)-1-hydroxyethyl)piperazine-2,5-dione (5.9 g, 0.03 mol) in BH₃ THF solution (200 mL) was stirred at room temperature for 2 hours, then stirred reflux for overnight. The reaction mixture was cooled to 0° C. MeOH (60 mL) was added gradually, followed by 5M HCl (16 mL) and the reaction mixture was stirred at reflux for 2 hours. The reaction mixture was cooled to room temperature. The resulting suspension was filtered to give the titled compound (3.2 g, 45%). MS: M/e 159 (M+1)⁺.

### Step D: tert-butyl (2R,5R)-2-ethyl-5-((R)-1-hydroxyethyl)piperazine-1-carboxylate

To a solution of (R)-1-((2R,5R)-5-ethylpiperazin-2-yl)ethan-1-ol (1 g, 4.4 mmol), Et₃N (1.3 g, 13.2 mmol) in MeOH (10 mL) was added Boc-anhydride (2.2 g, 10 mmol) over a period of 15 min at room temperature. The reaction mixture was heating at 50° C. overnight. The reaction mixture was concentrated. The resulting residue was dissolved in EtOH (40 mL) and a solution of NaOH (880 mg, 22 mmol) in water (20 mL) was added. The reaction mixture was heated at 100° C. for 16 hours. The reaction solvent was removed under reduced pressure. The resulting residue was dissolved in DCM:MeOH (10:1, 20 mL) and filtered. The resulting filtrates were concentrated to give the titled compound (820 mg, 94%).

### Step E: tert-butyl (2R,5R)-2-ethyl-5-((R)-1-hydroxyethyl)-4-(4-methyl-5-oxo-2-(tetrahydro-2H-pyran-2-yl)-4,5-dihydro-2H-pyrazolo[4,3-b]pyridin-7-yl)piperazine-1-carboxylate

A mixture of 4-methyl-5-oxo-2-(tetrahydro-2H-pyran-2-yl)-4,5-dihydro-2H-pyrazolo[4,3-b]pyridin-7-yl trifluoromethanesulfonate (270 mg, 0.7 mmol), tert-butyl (2R,5R)-2-ethyl-5-((R)-1-hydroxyethyl)piperazine-1-carboxylate (260 mg, 1 mmol) and DIPEA (450 mg, 3.5 mmol) in CH₃CN (3 mL) was heated to 90° C. for overnight under N₂ atmosphere. The reaction solvent was removed under vacuum. The resulting residue was purified by flash column chromatography (DCM:MeOH=15:1) to give the titled compound (110 mg, 32%). MS: M/e 490 (M+1)⁺.

### Step F: 7-((2R,5R)-5-ethyl-2-((R)-1-hydroxyethyl)piperazin-1-yl)-4-methyl-2-(tetrahydro-2H-pyran-2-yl)-2,4-dihydro-5H-pyrazolo[4,3-b]pyridin-5-one

To a solution of tert-butyl (2R,5R)-2-ethyl-5-((R)-1-hydroxyethyl)-4-(4-methyl-5-oxo-2-(tetrahydro-2H-pyran-2-yl)-4,5-dihydro-2H-pyrazolo[4,3-b]pyridin-7-yl)piperazine-1-carboxylate (110 mg, 0.2 mmol) in DCM (5 mL) was added TFA (1 mL) at room temperature. The resulting mixture was stirred at room temperature for 2 hours. The reaction mixture was neutralized by NaOH aq·(1 N) and extracted with DCM (20 mL×2). The organic layers were concentrated to give the titled compound (80 mg, crude). MS: M/e 390 (M+1)⁺.

### Step G: 7-((2R,5R)-5-ethyl-2-((R)-1-hydroxyethyl)-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of 7-((2R,5R)-5-ethyl-2-((R)-1-hydroxyethyl)piperazin-1-yl)-4-methyl-2-(tetrahydro-2H-pyran-2-yl)-2,4-dihydro-5H-pyrazolo[4,3-b]pyridin-5-one (80 mg, 0.2 mmol), 6-(1-chloroethyl)quinoxaline (190 mg, 1.0 mmol) and DIPEA (130 mg, 1.0 mmol) in DMSO (2 mL) was heated to 120° C. in a microwave for 2 hours. The reaction was cooled to room temperature. The mixture was extracted with EA and brine. The organic layers were concentrated and purified by Prep-TLC (DCM:MeOH=10:1) to give the titled compound (10 mg, 2%). MS: M/e 546 (M+1)⁺.

### Step H: 2-(7-((2R,5R)-5-ethyl-2-((R)-1-hydroxyethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 7-((2R,5R)-5-ethyl-2-((R)-1-hydroxyethyl)-4-(1-(quinoxalin-6-yl) ethyl)piperazin-1-yl)-4-methyl-2-(tetrahydro-2H-pyran-2-yl)-2,4-dihydro-5H-pyrazolo[4,3-b]pyridin-5-one (110 mg, 0.2 mmol) in DCM (5 mL) was added TFA (1 mL) at room temperature. The resulting mixture was stirred at room temperature for 2 hours. The mixture was neutralized by aq·NaOH (1 N) and extracted with DCM (20 mL×2). The organic layers was concentrated to dryness. The resulting residue was dissolved in DMF (3 mL), then K₂CO₃ (13 mg) and Iodoacetonitrile (16 mg) was added. The reaction mixture was stirred at room temperature for 2 hours. The reaction mixture was quenched with H₂O and extracted with EA (20 mL×2).

The organic layers were concentrated and the resulting residue was purified by Prep-HPLC to give the titled compound (0.21 mg). ¹H NMR (400 MHz, CD₃OD) δ 8.89-8.87 (m, 2H), 8.50 (br s, 1H), 8.14-7.91 (m, 4H), 5.49-5.47 (m, 2H), 5.35-5.33 (m, 1H), 4.66 (s, 3H), 4.06-4.04 (m, 0.5H), 3.87-3.85 (m, 0.5H), 3.47 (s, 3H), 2.80-2.74 (m, 2H), 2.22-2.17 (m, 1H), 2.03-2.02 (m, 1H), 1.65-1.55 (m, 2H), 1.49-1.42 (m, 2H), 0.91-0.60 (m, 7H). MS: M/e 465 (M+1)⁺.

### Compound A260: 2-(7-((2S,5R)-2,5-dimethyl-4-((R)-1-(quinoxalin-6-yl)ethyl)piperazin-1-yl)-5-oxo-4,5-dihydro-2H-pyrazolo[4,3-b]pyridin-2-yl)acetonitrile, or 2-(7-((2S,5R)-2,5-dimethyl-4-((S)-1-(quinoxalin-6-yl)ethyl)piperazin-1-yl)-5-oxo-4,5-dihydro-2H-pyrazolo[4,3-b]pyridin-2-yl)acetonitrile

### Step A: 7-((2S,5R)-2,5-dimethyl-4-((R)-1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-b]pyridin-5-one, or 7-((2S,5R)-2,5-dimethyl-4-((S)-1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-b]pyridin-5-one

To a solution of Intermediate 7 (361 mg, 0.73 mmol) and Intermediate 1a (200 mg, 0.73 mmol, optical isomer) in CH₃CN (5 mL) was added DIPEA (190 mg, 1.46 mmol). Then the mixture was heated at 90° C. under N₂ for 18 h. The mixture was cooled to room temperature, diluted with water (50 mL), extracted with EA (80 mL×3). The combined organic layers were washed with brine, dried over Na₂SO₄, filtered and concentrated to dryness. The resulting residue was purified by flash column chromatography (DCM:MeOH=15:1) to give titled compound (356 mg, 80%). MS: M/e 608 (M+1)⁺.

### Step B: 7-((2S,5R)-2,5-dimethyl-4-((R)-1-(quinoxalin-6-yl)ethyl)piperazin-1-yl)-2,4-dihydro-5H-pyrazolo[4,3-b]pyridin-5-one, or 7-((2S,5R)-2,5-dimethyl-4-((S)-1-(quinoxalin-6-yl)ethyl)piperazin-1-yl)-2,4-dihydro-5H-pyrazolo[4,3-b]pyridin-5-one

To a solution of 7-((2S,5R)-2,5-dimethyl-4-((R)-1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-b]pyridin-5-one, or 7-((2S,5R)-2,5-dimethyl-4-((S)-1-(quinoxalin-6-yl)ethyl)piperazin-1-yl)-4-(4-methoxybenzyl)-2-(tetrahydro-2H-pyran-2-yl)-2,4-dihydro-5H-pyrazolo[4,3-b]pyridin-5-one (350 mg, 0.576 mmol) in TFA (1 mL) was added trifluoromethanesulfonic acid (6 mL). The reaction was heated at 60° C. for 18 hours. The resulting mixture was cooled to room temperature. The reaction mixture was quenched with ice-water, basified with saturated Na₂CO₃ solution to pH 7-8, extracted with DCM:IPA (4:1, 80 mL×4). The combined organic layers were washed with brine, dried over Na₂SO₄, filtered and concentrated to dryness. The resulting residue was purified by Prep-TLC to give the titled compound (100 mg, 43%). MS: M/e 404 (M+1)⁺.

### Step C: 2-(7-((2S,5R)-2,5-dimethyl-4-((R)-1-(quinoxalin-6-yl)ethyl)piperazin-1-yl)-5-oxo-4,5-dihydro-2H-pyrazolo[4,3-b]pyridin-2-yl)acetonitrile, or 2-(7-((2S,5R)-2,5-dimethyl-4-((S)-1-(quinoxalin-6-yl)ethyl)piperazin-1-yl)-5-oxo-4,5-dihydro-2H-pyrazolo[4,3-b]pyridin-2-yl)acetonitrile

To a solution of 7-((2S,5R)-2,5-dimethyl-4-((R)-1-(quinoxalin-6-yl)ethyl)piperazin-1-yl)-2,4-dihydro-5H-pyrazolo[4,3-b]pyridin-5-one, or 7-((2S,5R)-2,5-dimethyl-4-((S)-1-(quinoxalin-6-yl)ethyl)piperazin-1-yl)-2,4-dihydro-5H-pyrazolo[4,3-b]pyridin-5-one (100 mg, 0.248 mmol) and K₂CO₃ (70 mg, 0.5 mmol) in DMF (2 mL) was added 2-chloroacetonitrile (46 mg, 0.278 mmol). The reaction was stirred at room temperature for 16 h. The reaction mixture was diluted with water, extracted with EA (80 mL×3), washed with brine, dried over Na₂SO₄, filtered, and concentrated to dryness. The resulting residue was purified by Prep-TLC (DCM:MeOH=12:1) to give the titled compound (24 mg, 21%). ¹HNMR (400 MHz, CD₃OD) δ 8.88 (d, J=1.6 Hz, 1H), 8.87 (d, J=1.2 Hz, 1H), 8.15-8.03 (m, 3H), 7.74 (s, 1H), 5.49 (s, 1H), 5.44 (s, 2H), 5.03-4.89 (m, 1H), 4.45-4.26 (m, 1H), 4.03-3.92 (m, 1H), 3.53-3.44 (m, 1H), 3.14-3.04 (m, 1H), 2.98-2.86 (m, 2H), 1.50-1.41 (m, 6H), 1.06 (d, J=6.8 Hz, 3H) ppm MS: M/e 443 (M+1)⁺.

### Compound A261: 2-(7-((2R,5R)-2-(difluoromethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: tert-butyl (2R,5R)-4-benzyl-5-(hydroxymethyl)-2-methylpiperazine-1-carboxylate

A mixture of tert-butyl (2R,5R)-5-(hydroxymethyl)-2-methylpiperazine-1-carboxylate (2.3 g, 10 mmol) and benzaldehyde (1.23 g, 12 mmol) in CH₂Cl₂ (60 mL) was stirred for 2 hours, then NaBH(OAc)₃ (4.24 g, 20 mmol) was added. After the addition, the reaction mixture was stirred over a weekend. H₂O (50 mL) was added to the mixture and extracted with CH₂Cl₂ (60 mL×3). The combined organic layers were washed with brine, dried over Na₂SO₄ and concentrated to dryness. The resulting residue was purified by flash column chromatography to give the titled compound (2.7 g, 84%). MS: M/e 321 (M+1)⁺.

### Step B: tert-butyl (2R,5R)-4-benzyl-5-formyl-2-methylpiperazine-1-carboxylate

To a stirred solution of the product of oxalyl dichloride (2.14 g, 16.88 mmol) in CH₂Cl₂ (10 mL) was added dropwise a solution of DMSO (2.6 g, 33.76 mmol) in CH₂Cl₂ (10 mL) at −78° C. After stirred for 30 min, a solution of tert-butyl (2R,5R)-4-benzyl-5-(hydroxymethyl)-2-methylpiperazine-1-carboxylate (2.7 g, 8.44 mmol) in CH₂Cl₂ (20 mL) was added dropwise at −78° C. over a period of 20 minutes, then stirred for another 30 minutes, followed by Et₃N (6.82 g, 67.52 mmol) was added dropwise at −78° C. and stirred for 30 minutes at −78° C. The reaction was quenched with H₂O at −70° C., after warmed to room temperature, the mixture was extracted with CH₂Cl₂ (50 mL×2). The combined organic layers were washed with brine, dried over Na₂SO₄, concentrated to give the residue, which was treated with H₂O (20 mL) and extracted with PE:TBME (30 mL×3, 1:1, v/v). The combined organic layers were washed with brine, dried over Na₂SO₄, concentrated to give the titled compound (2.3 g, 86%), which was used to the next step directly. ¹H NMR (400 MHz, DMSO-d6) δ 9.76 (s, 1H), 7.36-7.21 (m, 5H), 4.21 (d, J=13.6 Hz, 1H), 4.05 (s, 1H), 3.96 (s, 2H), 3.42 (d, J=4.4 Hz, 1H), 3.24 (dd, J=13.6, 5.2 Hz, 1H), 3.01 (dd, J=11.6, 4.4 Hz, 1H), 2.37 (dd, J=11.6, 2.4 Hz, 1H), 1.37 (s, 9H), 1.13 (d, J=6.8 Hz, 3H) ppm MS: M/e 319 (M+1)⁺.

### Step C: tert-butyl (2R,5R)-4-benzyl-5-(difluoromethyl)-2-methylpiperazine-1-carboxylate

To a stirred solution of tert-butyl (2R,5R)-4-benzyl-5-formyl-2-methylpiperazine-1-carboxylate (2.3 g, 7.23 mmol) in CH₂Cl₂ (20 mL) was added dropwise a solution of DAST (2.32 g, 14.5 mmol) in CH₂Cl₂ (10 mL) at 0° C. After the addition, the reaction was stirred for an hour. The reaction mixture was quenched with NaHCO₃ aq. and extracted with CH₂Cl₂ (30 mL×2). The combined organic layers were washed with brine, dried over Na₂SO₄ and concentrated to dryness. The resulting residue was purified by flash column chromatography to give the titled compound (1.33 g, 54%). MS: M/e 341 (M+1)⁺.

### Step D: tert-butyl (2R,5R)-5-(difluoromethyl)-2-methylpiperazine-1-carboxylate

To a stirred solution of tert-butyl (2R,5R)-4-benzyl-5-(difluoromethyl)-2-methylpiperazine-1-carboxylate (1.3 g, 3.85 mmol) in IPA/AcOH (30 mL/2 mL) was added Pd/C (300 mg, 10% in water). After the addition, the reaction mixture was stirred overnight under H₂ (4 atm). The reaction mixture was filtered. The filtrate was concentrated. The resulting residue was purified by flash column chromatography to give the titled compound (820 mg, 86%). MS: M/e 195 (M−56+1)⁺.

### Step E: tert-butyl (2R,5R)-5-(difluoromethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate

A mixture of the product of Intermediate 2 (55 mg, 0.22 mmol), tert-butyl (2R,5R)-5-(difluoromethyl)-2-methylpiperazine-1-carboxylate (55 mg, 0.22 mmol), Pd₂(dba)₃ (37 mg, 0.04 mmol), XPhos (38 mg, 0.08 mmol) and Cs₂CO₃ (215 mg, 0.66 mmol) in toluene (5 mL) was stirred at 100° C. overnight under N₂. The reaction mixture was diluted with EA (30 mL), washed with brine, dried over Na₂SO₄ and concentrated to dryness. The resulting residue was purified by Prep-HPLC to give the titled compound (41 mg, 19%). MS: M/e 482 (M+1)⁺.

### Step F: 7-((2R,5R)-2-(difluoromethyl)-5-methylpiperazin-1-yl)-4-methyl-2-(tetrahydro-2H-pyran-2-yl)-2,4-dihydro-5H-pyrazolo[4,3-b]pyridin-5-one

To a stirred solution of tert-butyl (2R,5R)-5-(difluoromethyl)-2-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (41 mg, 0.085 mmol) in CH₂Cl₂ (5 mL) was added TFA (1.5 mL). After the addition, the reaction mixture was stirred for 2 hours. The reaction mixture was basified to pH=8-9 with aq·NaHCO₃, then extracted with CH₂Cl₂ (10 mL×3). The combined organic layers were washed with brine, dried over Na₂SO₄, concentrated to give the titled compound (28 mg, 85%). MS: M/e 382 (M+1)⁺.

### Step G: 7-((2R,5R)-2-(difluoromethyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of 7-((2R,5R)-2-(difluoromethyl)-5-methylpiperazin-1-yl)-4-methyl-2-(tetrahydro-2H-pyran-2-yl)-2,4-dihydro-5H-pyrazolo[4,3-b]pyridin-5-one (28 mg, 0.073 mmol), 1-(quinoxalin-6-yl)ethan-1-ol (25.6 mg, 0.147 mmol), (cyanomethyl)trimethylphosphonium iodide (53.2 mg, 0.219 mmol) and DIPEA (94 mg, 0.73 mmol) in CH₃CN (3 mL) was stirred at 100° C. overnight in a sealed tube. The reaction mixture was diluted with EA (15 mL), washed with H₂O, brine, dried over Na₂SO₄ and concentrated to dryness. The resulting residue was purified by Pre-TLC (CH₂Cl₂:MeOH=10:1) to give the titled compound (10 mg, 26%). MS: M/e 538 (M+1)⁺.

### Step H: 2-(7-((2R,5R)-2-(difluoromethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile

To a stirred solution of 7-((2R,5R)-2-(difluoromethyl)-5-methyl-4-(1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (10 mg, 0.019 mmol) in MeOH (3 mL) was added HCl (2 mL, 4 M in 1,4-dioxane). Then the reaction mixture was stirred overnight. The reaction mixture was concentrated to give the residue, which was dissolved in DMF/H₂O (3 mL/0.5 mL) and K₂CO₃ (7.8 mg, 0.057 mmol) was added, followed by 2-iodoacetonitrile (6.27 mg, 0.038 mmol). Then the reaction was stirred for 4 hours. The reaction mixture was poured into H₂O (15 mL) and extracted with EA (15 mL×2). The combined organic layers were washed with brine, dried over Na₂SO₄ and concentrated to dryness. The resulting residue was purified by Prep-HPLC to give the titled compound (0.24 mg). ¹H NMR (400 MHz, CD₃OD) δ 8.92-8.84 (m, 2H), 8.16-7.93 (m, 4H), 6.62-6.32 (m, 1H), 5.67 (d, J=30.8 Hz, 1H), 5.48 (d, J=24 Hz, 1H), 4.62 (s, 1H), 4.04-3.49 (m, 4H), 3.45 (s, 3H), 3.22-3.15 (m, 0.5H), 3.03-2.93 (m, 1.5H), 2.50 (d, J=13.2 Hz, 1H), 1.50-1.43 (m, 3H), 1.24-1.04 (m, 3H) ppm. MS: M/e 493 (M+1)⁺.

### Compound A262: 2-(7-((2S,5R)-4-(2,2-difluoro-1-(quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 2,2-difluoro-1-(quinoxalin-6-yl)ethan-1-ol

To a solution of quinoxaline-6-carbaldehyde (1.58 g, 10 mmol) and cesium fluoride (200 mg, 1.32 mmol) in DMF (10 mL) was added dropwise (difluoromethyl)trimethylsilane (2.48 g, 20 mmol) under N₂. The reaction was stirred at room temperature for 16 hours. The reaction was quenched with 2N HCl solution (10 mL), diluted with water, extracted with EA (60 mL×3), washed with brine, dried over Na₂SO₄, filtered, and concentrated to dryness. The resulting residue was purified by flash column chromatography (PE/EA) to give titled compound (500 mg, 23%). ¹HNMR (400 MHz, DMSO-d₆) δ 8.97 (d, J=2.0 Hz, 2H), 8.19-8.10 (m, 2H), 7.96-7.90 (m, 1H), 6.54 (d, J=5.6 Hz, 1H), 6.36-6.03 (m, 1H), 5.19-5.05 (m, 1H) ppm. MS: M/e 211 (M+1)⁺.

### Step B: tert-butyl (2S,5R)-4-(2,2-difluoro-1-(quinoxalin-6-yl)ethyl)-2,5-dimethylpiperazine-1-carboxylate

A sealed tube was charged with 2,2-difluoro-1-(quinoxalin-6-yl)ethan-1-ol (105 mg, 0.5 mmol), tert-butyl (2S,5R)-2,5-dimethylpiperazine-1-carboxylate (160 mg, 0.75 mmol), (cyanomethyl)trimethylphosphonium iodide (364 mg, 1.5 mmol), DIPEA (258 mg, 2 mmol) and acetonitrile (3 ml). The mixture was stirred at 100° C. for overnight, cooled to RT. Water was added to quench the reaction and the aqueous was extracted with EA. The combined organic layers were washed with brine, dried over Na₂SO₄, and concentrated to dryness. The resulting residue was purified by Prep-TLC(EA:PE=1:1) to give the titled compound (45 mg, crude). MS: M/e 407 (M+1)⁺.

### Step C: 6-(1-((2R,5S)-2,5-dimethylpiperazin-1-yl)-2,2-difluoroethyl)quinoxaline

To the tert-butyl (2S,5R)-4-(2,2-difluoro-1-(quinoxalin-6-yl)ethyl)-2,5-dimethylpiperazine-1-carboxylate (50 mg) in DCM (2 mL) was added TFA (1 ml). The mixture was stirred at RT for 2 hours. The mixture was concentrated to dryness and diluted with water. The mixture was extracted with EA. The aqueous was adjusted pH to 12-13 with saturated sodium carbonate solution and extracted with DCM:MeOH(10:1). The combined organic layers were washed with brine, dried over Na₂SO₄, and concentrated to dryness. The resulting residue (30 mg, 81%) was used to the next step directly. MS: M/e 307 (M+1)⁺.

### Step D: 7-((2S,5R)-4-(2,2-difluoro-1-(quinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

To the 6-(1-((2R,5S)-2,5-dimethylpiperazin-1-yl)-2,2-difluoroethyl)quinoxaline (30 mg, 0.1 mmol) in acetonitrile (2 mL) was added Intermediate 2 (76 mg, 0.2 mmol) and DIPEA (65 mg, 0.5 mmol). The resulting mixture was stirred at 100° C. for overnight in a sealed tube. The mixture was concentrated to dryness. The reaction was quenched with water, extracted with EA, washed with brine, dried over Na₂SO₄, filtered, and concentrated to dryness. The resulting residue was purified by flash column chromatography (MeOH/DCM) to give the titled compound (40 mg, 74%). MS: M/e 538 (M+1)⁺.

### Step E: 7-((2S,5R)-4-(2,2-difluoro-1-(quinoxalin-6-yl)ethyl)-2,5-dimethylpiperazin-1-yl)-4-methyl-2,4-dihydro-5H-pyrazolo[4,3-b]pyridin-5-one

To a solution of 7-((2S,5R)-4-(2,2-difluoro-1-(quinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (40 mg) in MeOH (1.5 mL) was added HCl (2 ml, 4 M in 1,4-dioxane). The reaction was stirred at room temperature for 4 hours. The solvent was removed under reduced pressure. The resulting residue (30 mg, crude) was used directly to next step directly without further purification. MS: M/e 454 (M+1)⁺.

### Step F: 2-(7-((2S,5R)-4-(2,2-difluoro-1-(quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 7-((2S,5R)-4-(2,2-difluoro-1-(quinoxalin-6-yl)ethyl)-2,5-dimethylpiperazin-1-yl)-4-methyl-2,4-dihydro-5H-pyrazolo[4,3-b]pyridin-5-one (30 mg, crude) in DMF (2 mL) was added potassium carbonate (55 mg, 0.4 mmol) and 2-iodoacetonitrile (25 mg, 0.15 mmol). The resulting mixture was stirred at room temperature overnight. The reaction mixture was diluted with water, extracted with EA, washed with brine, dried over Na₂SO₄, filtered, and concentrated to dryness. The resulting residue was purified by Prep-TLC(DCM:MeOH=13:1) to give the titled compound (1 mg, 2%). ¹H NMR (400 MHz, CD₃OD) δ 8.92 (d, J=4.7 Hz, 2H), 8.21 (s, 1H), 8.14 (dd, J=12.5, 8.6 Hz, 1H), 8.06 (dd, J=13.8, 6.2 Hz, 1H), 7.93 (s, 1H), 6.60-6.25 (m, 1H), 5.57 (s, 1H), 5.47 (s, 2H), 4.26 (s, 2H), 3.70 (s, 1H), 3.43 (s, 3H), 2.96 (s, 2H), 2.51-2.30 (m, 2H), 1.43 (d, J=6.6 Hz, 3H), 1.07 (d, J=6.5 Hz, 3H) ppm. MS: M/e 493 (M+1)⁺.

### Compound A263: 2-(3-chloro-7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-b]pyridin-2-yl)acetonitrile, or 2-(3-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile

### Step A: methyl 5-chloro-4-(N-methylacetamido)-1-(tetrahydro-2H-pyran-2-yl)-1H-pyrazole-3-carboxylate

To a solution of methyl 4-(N-methylacetamido)-1-(tetrahydro-2H-pyran-2-yl)-1H-pyrazole-3-carboxylate (2.24 g, 8 mmol) in DMF (8 mL) was added NCS (1.28 g, 9.6 mmol). The reaction was stirred at 40° C. for 16 h. The mixture was diluted with water, extracted with EA (80 mL×3), washed with brine, dried over Na₂SO₄, filtered, and concentrated to dryness. The resulting residue was purified by flash column chromatography (EA:PE=1:1) to give the titled compound (890 mg, 35%). MS: M/e 316 (M+1)⁺.

### Step B: 3-chloro-7-hydroxy-4-methyl-2-(tetrahydro-2H-pyran-2-yl)-2,4-dihydro-5H-pyrazolo[4,3-b]pyridin-5-one

To a solution of methyl 5-chloro-4-(N-methylacetamido)-1-(tetrahydro-2H-pyran-2-yl)-1H-pyrazole-3-carboxylate (1.07 g, 3.4 mmol) in THF (20 mL) was added drop w is a solution of LiHMDS (6.8 mL, 1 mol/L) at −70° C. The reaction was stirred for 1 h, quenched with water, warmed to RT slowly, extracted with EA (100 mL). The water layer was collected, treated with citric acid to pH 3-4, extracted with DCM:IPA (4:1, 60 mL×3). The combined organic layers were dried over Na₂SO₄, filtered, and concentrated to dryness. The resulting residue was purified by flash column chromatography (DCM:MEOH=10:1) and further subjected to slurry with EA/PE to give the titled compound (140 mg, 15%) MS: M/e 284 (M+1)⁺.

### Step C: 3-chloro-4-methyl-5-oxo-2-(tetrahydro-2H-pyran-2-yl)-4,5-dihydro-2H-pyrazolo[4,3-b]pyridin-7-yl trifluoromethanesulfonate

To a solution of 3-chloro-7-hydroxy-4-methyl-2-(tetrahydro-2H-pyran-2-yl)-2,4-dihydro-5H-pyrazolo[4,3-b]pyridin-5-one (140 mg, 0.494 mmol) in DCM (5 mL) was added DMAP (1 mg) and TEA (175 mg, 1.5 mmol) at 0° C. Then a solution of Tf₂O (140 mg, 0.5 mmol) in DCM (1 mL) was added dropwise. The reaction was stirred at 0° C. for 10 min. The mixture was diluted with water, extracted with DCM (60 mL×2), washed with brine, dried over Na₂SO₄, filtered, and concentrated to dryness. The resulting residue was used to next step directly. MS: M/e 416 (M+1)⁺.

### Step D: 3-chloro-7-((2S,5R)-2,5-dimethyl-4-((R)-1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one, or 3-chloro-7-((2S,5R)-2,5-dimethyl-4-((S)-1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of 3-chloro-4-methyl-5-oxo-2-(tetrahydro-2H-pyran-2-yl)-4,5-dihydro-2H-pyrazolo[4,3-b]pyridin-7-yl trifluoromethanesulfonate (crude) and 6-(1-((2R,5S)-2,5-dimethylpiperazin-1-yl)ethyl)quinoxaline (intermediate 1a) (90 mg, 0.33 mmol, optical isomer) in CH₃CN (4 mL) was added DIPEA (129 mg, 1 mmol). Then the mixture was heated at 90° C. under N₂ for 18 h. The mixture was cooled to room temperature, diluted with water (50 mL), extracted with EA (60 mL×2), washed with brine, dried over Na₂SO₄, filtered and concentrated to dryness. The resulting residue was purified by Prep-TLC (DCM:MeOH=15:1) to give the titled compound (50 mg, 19% for two steps). MS: M/e 536 (M+1)⁺.

### Step E: 3-chloro-7-((2S,5R)-2,5-dimethyl-4-((R)-1-(quinoxalin-6-yl)ethyl)piperazin-1-yl)-4-methyl-2,4-dihydro-5H-pyrazolo[4,3-b]pyridin-5-one, or 3-chloro-7-((2S,5R)-2,5-dimethyl-4-((S)-1-(quinoxalin-6-yl)ethyl)piperazin-1-yl)-4-methyl-2,4-dihydro-5H-pyrazolo[4,3-b]pyridin-5-one

To a solution of 3-chloro-7-((2S,5R)-2,5-dimethyl-4-((R)-1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one, or 3-chloro-7-((2S,5R)-2,5-dimethyl-4-((S)-1-(quinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (50 mg, 0.093) in DCM (1 mL) was added TFA (3 mL). The resulting mixture was stirred at room temperature overnight. The mixture was concentrated under reduced pressure, diluted with water, basified with saturated NaHCO₃ solution to pH 7-8, extracted with DCM (50 mL×3). The combined organic layers were washed with brine, dried over Na₂SO₄, filtered and concentrated to dryness. The resulting residue was purified by Prep-TLC (DCM:MeOH=10:1) to give titled compound (20 mg, 47%). MS: M/e 452 (M+1)⁺.

### Step F: 2-(3-chloro-7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-b]pyridin-2-yl)acetonitrile, or 2-(3-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile

To a solution of 3-chloro-7-((2S,5R)-2,5-dimethyl-4-((R)-1-(quinoxalin-6-yl)ethyl)piperazin-1-yl)-4-methyl-2,4-dihydro-5H-pyrazolo[4,3-b]pyridin-5-one, or 3-chloro-7-((2S,5R)-2,5-dimethyl-4-((S)-1-(quinoxalin-6-yl)ethyl)piperazin-1-yl)-4-methyl-2,4-dihydro-5H-pyrazolo[4,3-b]pyridin-5-one (20 mg, 0.044 mmol) and K₂CO₃ (12 mg, 0.088 mmol) in DMF (2 mL) was added 2-chloroacetonitrile (5 mg, 0.066 mmol). The reaction was stirred at room temperature overnight. The reaction mixture was diluted with water, extracted with EA (60 mL×2), washed with brine, dried over Na₂SO₄, filtered, and concentrated to dryness. The resulting residue was purified by Prep-TLC (DCM:MeOH=15:1) to give the titled compound (5 mg, 22%). 1HNMR (400 MHz, CD3OD) δ 8.90-8.85 (m, 2H), 8.14-8.01 (m, 3H), 5.59 (s, 1H), 5.51 (s, 2H), 4.86-4.80 (m, 1H), 4.25-4.10 (m, 1H), 4.02-3.93 (m, 1H), 3.68 (s, 3H), 3.49-3.42 (m, 1H), 3.13-3.02 (m, 1H), 2.98-2.84 (m, 2H), 1.44 (t, J=6.0 Hz, 6H), 1.06 (d, J=6.8 Hz, 3H) ppm. MS: M/e 491 (M+1)⁺.

### Compound A265: 2-(7-((2S,5R)-2,5-diethyl-4-(1-(4-fluoro-2-(trifluoromethyl)phenyl)ethyl)piperazin-1-yl)-5-oxo-4,5-dihydro-2H-pyrazolo[4,3-b]pyridin-2-yl)acetonitrile

### Step A: 7-((2S,5R)-2,5-diethyl-4-(1-(4-fluoro-2-(trifluoromethyl)phenyl)ethyl)piperazin-1-yl)-4-(3,4-dimethoxybenzyl)-2-(tetrahydro-2H-pyran-2-yl)-2,4-dihydro-5H-pyrazolo[4,3-b]pyridin-5-one

To a solution of (2R,5S)-2,5-diethyl-1-(1-(4-fluoro-2-(trifluoromethyl)phenyl)ethyl)piperazine (365 mg, 1.10 mmol) and Intermediate 8 (568 mg, 1.10 mmol) in CH₃CN (2 mL) at room temperature was added DIPEA (426 mg, 3.30 mmol). The mixture was stirred at 105° C. for 24 hours. Then the reaction mixture was concentrated under reduced pressure. The resulting residue was purified by flash column chromatography to give the titled compound (600 mg, 78%). MS: M/e 700 (M+1)⁺.

### Step B: 7-((2S,5R)-2,5-diethyl-4-(1-(4-fluoro-2-(trifluoromethyl)phenyl)ethyl)piperazin-1-yl)-2,4-dihydro-5H-pyrazolo[4,3-b]pyridin-5-one

To a solution of 7-((2S,5R)-2,5-diethyl-4-(1-(4-fluoro-2-(trifluoromethyl)phenyl)ethyl)piperazin-1-yl)-4-(3,4-dimethoxybenzyl)-2-(tetrahydro-2H-pyran-2-yl)-2,4-dihydro-5H-pyrazolo[4,3-b]pyridin-5-one (600 mg, 0.858 mmol) in TFA (5 mL) and TFOH (10 mL) was stirred at 70° C. for 36 hours. Then the mixture was concentrated under reduced pressure and basified by Na₂CO₃ (4M) to pH-10 and extracted with DCM (50 mL×3). The combined organic layer was washed with brine (20 mL×3), dried over Na₂SO₄ and concentrated under reduced pressure. The resulting residue was purified by flash column chromatography to give the titled compound (150 mg, 38%). MS: M/e 466 (M+1)⁺.

### Step C: 2-(7-((2S,5R)-2,5-diethyl-4-(1-(4-fluoro-2-(trifluoromethyl)phenyl)ethyl)piperazin-1-yl)-5-oxo-4,5-dihydro-2H-pyrazolo[4,3-b]pyridin-2-yl)acetonitrile

To a solution of 7-((2S,5R)-2,5-diethyl-4-(1-(4-fluoro-2-(trifluoromethyl)phenyl)ethyl)piperazin-1-yl)-2,4-dihydro-5H-pyrazolo[4,3-b]pyridin-5-one (150 mg, 0.323 mmol) and K₂CO₃ (134 mg, 0.969 mmol) in DMF (8 mL) at room temperature was added 2-iodoacetonitrile (59 mg, 0.355 mmol). The mixture solution was stirred at room temperature for 12 hours. Then the reaction mixture was quenched with saturated NaCl (20 mL) at room temperature and extracted with EA (30 mL×2). The combined organic layers were dried over Na₂SO₄ and concentrated under reduced pressure. The resulting residue was purified by Prep-HPLC to give the titled compound (23 mg, 14%). ¹H NMR (400 MHz, CDCl₃) δ 11.59 (s, 1H), 8.08-7.62 (m, 1H), 7.69 (s, 1H), 7.32-7.30 (m, 1H), 7.29-7.26 (m, 1H), 5.53 (s, 1H), 5.13 (s, 2H), 4.27-3.97 (m, 1H), 3.58-3.47 (m, 1H), 3.26-3.19 (m, 1H), 3.04-3.01 (m, 0.5H), 2.86-2.58 (m, 1H), 2.52-2.02 (m, 2.5H), 1.90-1.49 (m, 4H), 1.39-1.25 (m, 3H), 1.05-0.85 (m, 3H), 0.70-0.55 (m, 3H). MS: M/e 505 (M+1)⁺.

### Compound A266: 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-2H-pyrazolo[4,3-d]pyrimidin-2-yl)acetonitrile

### Step A: 7-((2S,5R)-4-(1-(4-fluoro-2-(trifluoromethyl)phenyl)ethyl)-2,5-dimethylpiperazin-1-yl)-4-methyl-2-(tetrahydro-2H-pyran-2-yl)-2,4-dihydro-5H-pyrazolo[4,3-d]pyrimidin-5-one

To a solution of Intermediate 9 (125 mg, 0.5 mmol) in CH₃CN (3 mL) was added DIPEA (129 mg, 1 mmol), followed by POCl₃ (135 mg, 1 mmol) and one drop of DMF. The reaction was stirred at 80° C. for 2 h. The reaction was cooled to room temperature, (2R,5S)-1-(1-(4-fluoro-2-(trifluoromethyl)phenyl)ethyl)-2,5-dimethylpiperazine (120 mg, 0.4 mmol) and DIPEA (645 mg, 5 mmol) were added. The resulting mixture was heated at 80° C. for 15 hours. The reaction was quenched with water, extracted with EA (60 mL×2), washed with brine, dried over Na₂SO₄, filtered, and concentrated to dryness. The resulting residue was purified by Prep-TLC (DCM:MeOH=15:1) to give the titled compound (15 mg, 5%). MS: M/e 537 (M+1)⁺.

### Step B: 7-((2S,5R)-4-(1-(4-fluoro-2-(trifluoromethyl)phenyl)ethyl)-2,5-dimethylpiperazin-1-yl)-4-methyl-2,4-dihydro-5H-pyrazolo[4,3-d]pyrimidin-5-one

To a solution of 7-((2S,5R)-4-(1-(4-fluoro-2-(trifluoromethyl)phenyl)ethyl)-2,5-dimethylpiperazin-1-yl)-4-methyl-2-(tetrahydro-2H-pyran-2-yl)-2,4-dihydro-5H-pyrazolo[4,3-d]pyrimidin-5-one (15 mg, 0.0279 mmol) in DCM (1 mL) was added TFA (4 mL). The resulting mixture was stirred at room temperature for 16 hours. The reaction mixture was concentrated under reduced pressure to give the titled compound (crude). MS: M/e 453 (M+1)⁺.

### Step C: 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-2H-pyrazolo[4,3-d]pyrimidin-2-yl)acetonitrile

To a solution of 7-((2S,5R)-4-(1-(4-fluoro-2-(trifluoromethyl)phenyl)ethyl)-2,5-dimethylpiperazin-1-yl)-4-methyl-2,4-dihydro-5H-pyrazolo[4,3-d]pyrimidin-5-one (crude) and K₂CO₃ (40 mg, 0.27 mmol) in DMF (2 mL) was added 2-chloroacetonitrile (5 mg, 0.055 mmol). The reaction was stirred at room temperature for 5 h. The reaction mixture was diluted with water, extracted with EA (60 mL×2), washed with brine, dried over Na₂SO₄, filtered, and concentrated to dryness. The resulting residue was purified by Prep-TLC (DCM:MeOH=15:1) to give the titled compound (5 mg, 36% for two steps). ¹HNMR (400 MHz, CD₃OD) δ 8.16-8.01 (m, 1H), 7.93 (d, J=8.4 Hz, 1H), 7.47-7.36 (m, 2H), 5.90-5.46 (m, 3H), 5.38-5.30 (m, 0.5H), 5.17-5.08 (m, 0.5H), 4.75-4.60 (m, 0.5H), 4.12-4.00 (m, 0.5H), 3.94-3.85 (m, 0.5H), 3.75-3.45 (m, 1H), 3.40 (s, 3H), 3.05-2.68 (m, 2H), 2.08-2.00 (m, 0.5H), 1.59 (d, J=6.8 Hz, 0.5H), 1.51 (d, J=6.8 Hz, 0.5H), 1.37-1.25 (m, 5H), 1.06 (d, J=6.4 Hz, 2H), 0.92 (d, J=6.4 Hz, 1H) ppm. MS: M/e 492 (M+1)⁺.

### Compound A267: 2-(7-((2S,5R)-4-(1-(4-fluoro-2-(trifluoromethyl)phenyl)ethyl)-2,5-dimethylpiperazin-1-yl)-3,4-dimethyl-5-oxo-4,5-dihydro-2H-pyrazolo[4,3-d]pyrimidin-2-yl)acetonitrile

### Step A: 7-chloro-4-methyl-2-(tetrahydro-2H-pyran-2-yl)-2,4-dihydro-5H-pyrazolo[4,3-d]pyrimidin-5-one

To a solution of Intermediate 9 (0.2 g, 0.8 mmol) in CH₃CN (5 mL) were added DIPEA (207 mg, 1.6 mmol) and POCl₃ (245 mg, 1.6 mmol) at RT. The reaction mixture was stirred at 80° C. for 2 hours. The reaction mixture was used to the next step directly without further purification.

### Step B: tert-butyl (2R,5S)-2,5-dimethyl-4-(4-methyl-5-oxo-2-(tetrahydro-2H-pyran-2-yl)-4,5-dihydro-2H-pyrazolo[4,3-d]pyrimidin-7-yl)piperazine-1-carboxylate

To a solution of (crude, 0.8 mmol) in CH₃CN (5 mL) were added DIPEA (1 g, 8 mmol) and tert-butyl (2R,5S)-2,5-dimethylpiperazine-1-carboxylate (516 mg, 2.4 mmol) at 80° C. overnight. The reaction mixture was concentrated to dryness. The resulting residue was purified by flash column chromatography (DCM/MeOH) to give the titled compound (270 mg). MS: M/e 447 (M+1)⁺.

### Step C: tert-butyl (2R,5S)-4-(3-bromo-4-methyl-5-oxo-2-(tetrahydro-2H-pyran-2-yl)-4,5-dihydro-2H-pyrazolo[4,3-d]pyrimidin-7-yl)-2,5-dimethylpiperazine-1-carboxylate

To a solution of tert-butyl (2R,5S)-2,5-dimethyl-4-(4-methyl-5-oxo-2-(tetrahydro-2H-pyran-2-yl)-4,5-dihydro-2H-pyrazolo[4,3-d]pyrimidin-7-yl)piperazine-1-carboxylate (270 mg, 0.605 mmol) in CH₃CN (10 mL) was added NBS (162 mg, 0.91 mmol). The resulting mixture was stirred at RT for 5 hours. The reaction solvent was removed under vacuum. The resulting residue was purified by flash column chromatography (DCM/MeOH) to give the titled compound (300 mg). MS: M/e 525 (M+1)⁺.

### Step D: tert-butyl (2R,5S)-4-(3,4-dimethyl-5-oxo-2-(tetrahydro-2H-pyran-2-yl)-4,5-dihydro-2H-pyrazolo[4,3-d]pyrimidin-7-yl)-2,5-dimethylpiperazine-1-carboxylate

To a solution of tert-butyl (2R,5S)-4-(3-bromo-4-methyl-5-oxo-2-(tetrahydro-2H-pyran-2-yl)-4,5-dihydro-2H-pyrazolo[4,3-d]pyrimidin-7-yl)-2,5-dimethylpiperazine-1-carboxylate (0.27 g, 0.52 mmol) in dioxane (10 mL) were added K₂CO₃ (142 mg, 1.04 mmol), Pd(dppf)Cl₂ (39 mg, 0.05 mmol) and 2,4,6-trimethyl-1,3,5-trioxane (2.08 mL, 1M, 2.08 mmol) at RT. The resulting mixture was stirred at 90° C. overnight. The reaction solvent was removed under vacuum. The resulting residue was purified by flash column chromatography (DCM/MeOH) to give the target compound (70 mg). MS: M/e 461 (M+1)⁺.

### Step E: 7-((2S,5R)-2,5-dimethylpiperazin-1-yl)-3,4-dimethyl-2-(tetrahydro-2H-pyran-2-yl)-2,4-dihydro-5H-pyrazolo[4,3-d]pyrimidin-5-one

To a solution of tert-butyl (2R,5S)-4-(3,4-dimethyl-5-oxo-2-(tetrahydro-2H-pyran-2-yl)-4,5-dihydro-2H-pyrazolo[4,3-d]pyrimidin-7-yl)-2,5-dimethylpiperazine-1-carboxylate (70 mg, 0.15 mmol) in DCM (4 mL) were added TFA (1 mL). The resulting mixture was stirred at RT for another 2 hours. The reaction solvent was removed under vacuum. The crude product was dissolved with water and adjusted to pH=9 with sat. NaHCO₃. The aqueous layer was extracted with (DCM:IPA=4:1) and the organic layers were concentrated to dryness. The resulting residue was purified by Prep-TLC (DCM:MeOH=10:1) to give the titled compound (40 mg). MS: M/e 361 (M+1)⁺.

### Step F: 7-((2S,5R)-4-(1-(4-fluoro-2-(trifluoromethyl)phenyl)ethyl)-2,5-dimethylpiperazin-1-yl)-3,4-dimethyl-2-(tetrahydro-2H-pyran-2-yl)-2,4-dihydro-5H-pyrazolo[4,3-d]pyrimidin-5-one

To a solution of 7-((2S,5R)-2,5-dimethylpiperazin-1-yl)-3,4-dimethyl-2-(tetrahydro-2H-pyran-2-yl)-2,4-dihydro-5H-pyrazolo[4,3-d]pyrimidin-5-one (40 mg, 0.07 mmol) in CH₃CN (4 mL) was added 1-(4-fluoro-2-(trifluoromethyl)phenyl)ethan-1-ol (45 mg, 0.22 mmol), (cyanomethyl)trimethylphosphonium iodide (71 mg, 0.29 mmol) and DIPEA (94 mg, 0.7 mmol). The resulting mixture was stirred at 100° C. overnight. The reaction mixture was concentrated to dryness. The resulting residue was purified by Prep-TLC (DCM:MeOH=15:1) to give the titled compound (25 mg, 40%). MS: M/e 551 (M+1)⁺.

### Step G: 7-((2S,5R)-4-(1-(4-fluoro-2-(trifluoromethyl)phenyl)ethyl)-2,5-dimethylpiperazin-1-yl)-3,4-dimethyl-2,4-dihydro-5H-pyrazolo[4,3-d]pyrimidin-5-one

To a solution of 7-((2S,5R)-4-(1-(4-fluoro-2-(trifluoromethyl)phenyl)ethyl)-2,5-dimethylpiperazin-1-yl)-3,4-dimethyl-2-(tetrahydro-2H-pyran-2-yl)-2,4-dihydro-5H-pyrazolo[4,3-d]pyrimidin-5-one (25 mg, 0.045 mmol) in MeOH(1 mL) was added HCl (2 mL, 4 M in 1,4-dioxane). The resulting mixture was stirred at RT for 2 hours. The reaction mixture was concentrated to give the titled compound (crude), which was used to the next step directly without further purification.

### Step F: 2-(7-((2S,5R)-4-(1-(4-fluoro-2-(trifluoromethyl)phenyl)ethyl)-2,5-dimethylpiperazin-1-yl)-3,4-dimethyl-5-oxo-4,5-dihydro-2H-pyrazolo[4,3-d]pyrimidin-2-yl)acetonitrile

To a solution of 7-((2S,5R)-4-(1-(4-fluoro-2-(trifluoromethyl)phenyl)ethyl)-2,5-dimethylpiperazin-1-yl)-3,4-dimethyl-2,4-dihydro-5H-pyrazolo[4,3-d]pyrimidin-5-one (crude, 0.05 mmol) crude in DMF (2 mL)/water (0.4 mL) was added K₂CO₃ (31 mg, 0.23 mmol). The resulting mixture was stirred at RT for 15 minutes, then added 2-iodoacetonitrile (38 mg, 0.225 mmol). The resulting mixture was stirred at RT for 1.5 hours. The reaction mixture was quenched by saturated NaCl aq. and extracted with EA. The organic layers were concentrated to give the residue containing Compound A267, which was further purified to give Compound A267a (0.84 mg) and Compound A267b (0.63 mg) by Prep-HPLC (Method A).

Compound A267a (the earlier peak): ¹H NMR (400 MHz, CD₃OD) δ 8.06 (t, J=6.8 Hz, 1H), 7.40 (t, J=9.2 Hz, 2H), 5.81-5.59 (m, 1H), 5.51 (t, J=12.6 Hz, 2H), 5.12-4.82 (m, 1H), 3.95-3.84 (m, 1H), 3.68 (s, 1H), 3.61 (s, 3H), 3.53-3.38 (m, 1H), 2.83-2.68 (m, 1H), 2.65 (s, 3H), 2.02 (d, J=11.9 Hz, 1H), 1.39-1.17 (m, 6H), 1.05 (d, J=6.4 Hz, 3H). MS: M/e 506 (M+1)⁺.

Compound A267b (the later peak): ¹H NMR (400 MHz, CD₃OD) δ 8.16-8.05 (m, 1H), 7.43 (t, J=8.8 Hz, 2H), 5.53-5.49 (m, 2H), 5.43-5.28 (m, 1H), 4.75-3.98 (m, 2H), 3.62 (t, J=6.8 Hz, 3H), 3.01-2.86 (m, 2H), 2.84-2.78 (s, 2H), 2.70 (d, J=13.9 Hz, 3H), 1.56-1.52 (m, 3H), 1.26 (d, J=4.5 Hz, 3H), 0.91 (d, J=6.4 Hz, 3H). MS: M/e 506 (M+1)⁺.

Compound A268: 2-(7-((2S,5R)-2,5-dimethyl-4-(1-(quinoxalin-6-yl-2,3-d2)ethyl)piperazin-1-yl)-4-methyl-5-oxo-4,5-dihydro-2H-pyrazolo[4,3-b]pyridin-2-yl)acetonitrile

### Step A: methyl quinoxaline-6-carboxylate-2,3-d2

To a stirred solution of methyl 2,3-dichloroquinoxaline-6-carboxylate (1.27 g, 5 mmol) in THF (20 mL) was added PddppfCl₂ (283 mg, 0.5 mmol) and NaBD₄ (420 mg, 10 mmol), followed by DIPEA (1.29 g, 10 mmol). After the addition, the reaction mixture was stirred for 2 hours under N₂ at RT. The reaction mixture was quenched with CD₃OD (5 mL) and stirred for 5 minutes. The reaction mixture was poured into H₂O (50 mL), extracted with EA (30 mL×3). The combined organic layers were washed with brine, dried over Na₂SO₄, concentrated. The resulting residue was purified by flash column chromatography to give the titled compound (373 mg, 39%). ¹H NMR (400 MHz, DMSO-d6) δ 8.66 (d, J=1.6 Hz, 1H), 8.33 (dd, J=8.8, 1.6 Hz, 1H), 8.24 (d, J=8.8 Hz, 1H), 3.97 (s, 3H) ppm. MS: M/e 191 (M+1)⁺.

### Step B: quinoxaline-6-carboxylic-2,3-d2 acid

To a stirred solution of methyl quinoxaline-6-carboxylate-2,3-d2 (373 mg, 1.96 mmol) in THF (10 mL) was added a solution of LiOH·H₂O (165 mg, 3.92 mmol) in H₂O (2 mL). After the addition, the reaction mixture was stirred overnight. The reaction mixture was acidified to pH=3-4 with HCl aq. and extracted with EA (15 mL×3). The combined organic layers were washed with brine, dried over Na₂SO₄, concentrated to give the titled compound (334 mg, 97%). MS: M/e 177 (M+1)⁺.

### Step C: N-methoxy-N-methylquinoxaline-6-carboxamide-2,3-d2

A mixture of quinoxaline-6-carboxylic-2,3-d2 acid (334 mg, 1.9 mmol), N,O-dimethylhydroxylamine hydrochloride (222.3 mg, 2.28 mmol), HATU (8.71 mg, 2.28 mmol) and DIPEA (490 mg, 3.8 mmol) in DMF (10 mL) was stirred for 3 days. The reaction mixture was poured into H₂O (20 mL) and extracted with EA (15 mL×2). The combined organic layers were washed with brine, dried over Na₂SO₄, concentrated. The resulting residue was purified by flash column chromatography to give the titled compound (296 mg, 71%). MS: M/e 220 (M+1)⁺.

### Step D: 1-(quinoxalin-6-yl-2,3-d2)ethan-1-one

To a stirred solution of N-methoxy-N-methylquinoxaline-6-carboxamide-2,3-d2 (110 mg, 0.5 mmol) in THF (10 mL) was added dropwise MeMgBr (3.0 M, 0.2 mL, 0.6 mmol) at 0° C. After the addition, the reaction was stirred for 10 minutes. The reaction mixture was quenched with NH₄Cl aq. and extracted with EA (10 mL×2). The combined organic layers were washed with brine, dried over Na₂SO₄, concentrated to dryness. The resulting residue was purified by flash column chromatography to give the titled compound (48 mg, 55%). MS: M/e 175 (M+1)⁺.

### Step E: 1-(quinoxalin-6-yl-2,3-d2)ethan-1-ol

To a stirred solution of 1-(quinoxalin-6-yl-2,3-d2)ethan-1-one (48 mg, 0.276 mmol) in EtOH (5 mL) was added NaBH₄ (8.4 mg, 0.22 mmol). After then, the reaction was stirred for 20 minutes. The reaction mixture was poured into H₂O (10 mL) and extracted with EA (10 mL×2). The combined organic layers were washed with brine, dried over Na₂SO₄ and concentrated to give the titled compound (44 mg, 91%). MS: M/e 177 (M+1)⁺.

### Step F: 7-((2S,5R)-2,5-dimethyl-4-(1-(quinoxalin-6-yl-2,3-d2)ethyl)piperazin-1-yl)-4-methyl-2-(tetrahydro-2H-pyran-2-yl)-2,4-dihydro-5H-pyrazolo[4,3-b]pyridin-5-one

A mixture of Intermediate 10B (69 mg, 0.2 mmol), 1-(quinoxalin-6-yl-2,3-d2)ethan-1-ol (44 mg, 0.25 mmol), (cyanomethyl)trimethylphosphonium iodide (146 mg, 0.6 mmol) and DIPEA (258 mg, 2 mmol) in CH₃CN (4 mL) was stirred at 100° C. overnight in a sealed tube. The reaction mixture was diluted with EA (15 mL), washed with H₂O, brine, dried over Na₂SO₄, concentrated. The resulting residue was purified by Pre-TLC (CH₂Cl₂/MeOH=10:1) to give the titled compound (20 mg, 20%). MS: M/e 504 (M+1)⁺.

### Step G: 2-(7-((2S,5R)-2,5-dimethyl-4-(1-(quinoxalin-6-yl-2,3-d2)ethyl)piperazin-1-yl)-4-methyl-5-oxo-4,5-dihydro-2H-pyrazolo[4,3-b]pyridin-2-yl)acetonitrile

To a stirred solution of 7-((2S,5R)-2,5-dimethyl-4-(1-(quinoxalin-6-yl-2,3-d2)ethyl)piperazin-1-yl)-4-methyl-2-(tetrahydro-2H-pyran-2-yl)-2,4-dihydro-5H-pyrazolo[4,3-b]pyridin-5-one (20 mg, 0.04 mmol) in MeOH (3 mL) was added HCl (2 mL, 4 M in 1,4-dioxane). Then the reaction mixture was stirred overnight. The reaction mixture was concentrated to give the residue. To a solution of the resulting residue in DMF/H₂O (3 mL/0.5 mL) was added K₂CO₃ (16.6 mg, 0.12 mmol), followed by 2-iodoacetonitrile (13.3 mg, 0.08 mmol). Then the reaction was stirred for 4 hours. The reaction mixture was poured into H₂O (15 mL) and extracted with EA (10 mL×2). The combined organic layers were washed with brine, dried over Na₂SO₄ and concentrated. The resulting residue was further purified by Pre-TLC (CH₂Cl₂:MeOH=10:1) to give the titled compound (6 mg). ¹H NMR (400 MHz, CD₃OD) δ 8.14-8.01 (m, 3H), 7.93 (d, J=1.6 Hz, 1H), 5.57 (s, 1H), 5.47 (d, J=3.6 Hz, 2H), 4.60 (s, 1H), 4.28 (s, 0.5H), 4.02-3.80 (m, 1H), 3.74-3.65 (m, 1H), 3.51-3.45 (m, 0.5H), 3.43 (s, 3H), 3.14-3.05 (m, 0.5H), 2.98-2.82 (m, 2H), 2.22 (d, J=12.0 Hz, 0.5H), 1.51-1.41 (m, 4.5H), 1.22 (t, J=7.2 Hz, 3H), 1.06 (d, J=6.4 Hz, 1.5H) ppm. MS: M/e 459 (M+1)⁺.

Compound A269: Mixture of 2-(7-((2S,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-dimethyl-4-(1-(2-methylquinoxalin-6-yl)ethyl)piperazin-1-yl)-4-methyl-5-oxo-4,5-dihydro-2H-pyrazolo[4,3-b]pyridin-2-yl)acetonitrile

### Step A: mixture of 7-((2S,5R)-2,5-dimethyl-4-(1-(3-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one and 7-((2S,5R)-2,5-dimethyl-4-(1-(2-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

A mixture of Intermediate 10B (345 mg, 2 mmol), a mixture of 1-(3-methylquinoxalin-6-yl)ethan-1-ol and 1-(2-methylquinoxalin-6-yl)ethan-1-ol (396 mg, 2 mmol), (cyanomethyl)trimethylphosphonium iodide (729 mg, 3 mmol) and DIPEA (1.29 g, 10 mmol) in CH₃CN (10 mL) was stirred at 100° C. overnight in a sealed tube. The reaction mixture was diluted with EA (50 mL), washed with H₂O, brine, dried over Na₂SO₄ and concentrated to dryness. The resulting residue was purified by flash column chromatography to give the titled compound (300 mg, 58%). MS: M/e 516 (M+1)⁺.

### Step B: mixture of 2-(7-((2S,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-dimethyl-4-(1-(2-methylquinoxalin-6-yl)ethyl)piperazin-1-yl)-4-methyl-5-oxo-4,5-dihydro-2H-pyrazolo[4,3-b]pyridin-2-yl)acetonitrile

The product of Step A (300 mg, 0.58 mmol) in MeOH (5 mL) was added HCl (3 mL, 4.0 M in 1,4-dioxane). Then the reaction mixture was stirred overnight. The reaction mixture was concentrated to give the residue, which was diluted with Na₂CO₃ aq. and extracted with CH₂Cl₂:IPA (3:1, v/v, 50 mL×3). The combined organic layers were washed with brine, dried over Na₂SO₄, concentrated to dryness. The resulting residue was purified by column chromatography to give the titled product, which is a mixture of 7-((2S,5R)-2,5-dimethyl-4-(1-(3-methylquinoxalin-6-yl)ethyl)piperazin-1-yl)-4-methyl-2,4-dihydro-5H-pyrazolo[4,3-b]pyridin-5-one and 7-((2S,5R)-2,5-dimethyl-4-(1-(2-methylquinoxalin-6-yl)ethyl)piperazin-1-yl)-4-methyl-2,4-dihydro-5H-pyrazolo[4,3-b]pyridin-5-one (110 mg, 44%). The product was dissolved in DMF (4 mL) and K₂CO₃ (141 mg, 1.02 mmol) was added, followed by 2-chloroacetonitrile (77 mg, 1.02 mmol). The reaction was stirred for 4 hours. The reaction mixture was poured into H₂O (15 mL) and extracted with EA (10 mL×3). The combined organic layers were washed with brine, dried over Na₂SO₄ and concentrated to dryness. The resulting residue was purified by flash column chromatography to give the titled Compound A269 (100 mg) as a mixture, which was further separated by three chiral prep-HPLC conditions into 4 isomers respectively, Compound A269a (14 mg), Compound A269b (18 mg), Compound A269c (5 mg) and Compound A269d (3 mg). The chiral separation conditions are shown below.

The First Prep-HPLC Condition

| Column | CHIRALPAK IC | |
|---|---|---|
| Column Size | 2 cm × 25 cm, 5 um | |
| Mobile Phase A | Hex:DCM = 3:1 | |
| Mobile Phase B | EtOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

The Second Prep-HPLC Condition

| Column | CHIRALPAK AD-H | |
|---|---|---|
| Column Size | 2 cm × 25 cm, 5 um | |
| Mobile Phase A | Hex | |
| Mobile Phase B | EtOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

The Third Prep-HPLC Condition

| Column | CHIRAL Cellulose-SB | |
|---|---|---|
| Column Size | 2 cm × 25 cm, 5 um | |
| Mobile Phase A | MTBE | |
| Mobile Phase B | EtOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A269a (the first peak, 2-(7-((2S,5R)-2,5-dimethyl-4-((S)-1-(2-methylquinoxalin-6-yl)ethyl)piperazin-1-yl)-4-methyl-5-oxo-4,5-dihydro-2H-pyrazolo[4,3-b]pyridin-2-yl)acetonitrile, or 2-(7-((2S,5R)-2,5-dimethyl-4-((R)-1-(2-methylquinoxalin-6-yl)ethyl)piperazin-1-yl)-4-methyl-5-oxo-4,5-dihydro-2H-pyrazolo[4,3-b]pyridin-2-yl)acetonitrile): ¹H NMR (400 MHz, CD₃OD) δ 8.80 (s, 1H), 8.04-7.96 (m, 3H), 7.92 (s, 1H), 5.56 (s, 1H), 5.46 (s, 2H), 4.88 (s, 1H), 4.27 (s, 1H), 3.94 (d, J=6.4 Hz, 1H), 3.49-3.45 (m, 1H), 3.43 (s, 3H), 3.12-3.03 (m, 1H), 2.96-2.85 (m, 2H), 2.77 (s, 3H), 1.54-1.38 (m, 6H), 1.05 (d, J=6.5 Hz, 3H) ppm. MS: M/e 471 (M+1)⁺.

Compound A269b (the second peak, 2-(7-((2S,5R)-2,5-dimethyl-4-((S)-1-(2-methylquinoxalin-6-yl)ethyl)piperazin-1-yl)-4-methyl-5-oxo-4,5-dihydro-2H-pyrazolo[4,3-b]pyridin-2-yl)acetonitrile, or 2-(7-((2S,5R)-2,5-dimethyl-4-((R)-1-(2-methylquinoxalin-6-yl)ethyl)piperazin-1-yl)-4-methyl-5-oxo-4,5-dihydro-2H-pyrazolo[4,3-b]pyridin-2-yl)acetonitrile): ¹H NMR (400 MHz, CD₃OD) δ 8.79 (s, 1H), 8.02 (s, 1H), 7.97 (s, 2H), 7.92 (s, 1H), 5.56 (s, 1H), 5.47 (s, 2H), 4.60 (s, 2H), 3.79 (q, J=6.4 Hz, 1H), 3.73-3.62 (m, 2H), 3.43 (s, 3H), 2.84 (dd, J=12.0, 4.0 Hz, 1H), 2.76 (s, 3H), 2.21 (d, J=12.0 Hz, 1H), 1.45 (d, J=6.4 Hz, 3H), 1.26-1.16 (m, 6H) ppm. MS: M/e 471 (M+1)⁺.

Compound A269c (the third peak, 2-(7-((2S,5R)-2,5-dimethyl-4-((R)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile): ¹H NMR (400 MHz, CD₃OD) δ 8.78 (s, 1H), 8.09-7.86 (m, 4H), 5.57 (s, 1H), 5.47 (s, 2H), 4.59 (s, 2H), 3.80 (q, J=6.4 Hz, 1H), 3.73-3.61 (m, 2H), 3.44 (s, 3H), 2.86 (dd, J=12.0, 4.0 Hz, 1H), 2.76 (s, 3H), 2.21 (d, J=12.0 Hz, 1H), 1.46 (d, J=6.4 Hz, 3H), 1.22 (dd, J=11.6, 6.4 Hz, 6H) ppm. MS: M/e 471 (M+1)⁺.

Compound A269d (the fourth peak, 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile): ¹HNMR (400 MHz, CD₃OD) δ 8.79 (s, 1H), 8.06 (d, J=8.8 Hz, 1H), 7.99-7.90 (m, 3H), 5.57 (s, 1H), 5.46 (s, 2H), 5.01-4.88 (m, 1H), 4.36-4.20 (m, 1H), 3.99-3.90 (m, 1H), 3.50-3.43 (m, 1H), 3.43 (s, 3H), 3.12-3.04 (m, 1H), 2.97-2.86 (m, 2H), 2.77 (s, 3H), 1.48-1.39 (m, 6H), 1.05 (d, J=6.4 Hz, 3H) ppm. MS: M/e 471 (M+1)⁺.

### Compound A269 (RH) 2-(7-((2S,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile

### Step A: 7-bromo-2-methylquinoxaline

7-bromo-2-chloroquinoxaline (20 g, 82 mmol) and Ferric acetylacetonate (2.9 g, 8.2 mmol) were dissolved in dry THF (150 mL). A solution of methyl magnesium bromide (3M in ethyl ether) (30 m, 91 mmol) was added dropwise at 0° C. After stirred for 1 hour, the reaction mixture was diluted with EA and quenched with 1M HC aq·. The organic layer was separated, washed with water and brine and dried over Na₂SO₄. The organic solvent was removed under vacuum. The resulting residue was further purified by flash column chromatography (PE:EA=5:1) to give the titled compound (14.5 g, 79%). ¹H NMR (400 MHz, DMSO-d₆) δ 8.89 (s, 1H), 8.22 (d, J=2.0 Hz, 1H), 8.00 (d, J=8.9 Hz, 1H), 7.91 (dd, J=8.9, 2.0 Hz, 1H), 2.72 (s, 3H). MS: M/e 223 (M+1)⁺.

### Step B: 1-(3-methylquinoxalin-6-yl)ethan-1-one

To a solution of 7-bromo-2-methylquinoxaline (22 g, 0.1 mol) and Cs₂CO₃ (48.6 g, 0.15 mol) in DMF/H₂O (300 mL) was added 1-(vinyloxy)butane (80 g, 0.8 mol), Pd(OAc)₂ (1.8 g, 0.08 mol) and DPPP (9.88 g, 0.24 mol). The mixture was stirred at 116° C. under N₂ for 16 hours. The reaction mixture was cooled to room temperature, treated with 2N HCl (175 mL) and stirred for 1 hour. The reaction mixture was diluted with water, extracted with EA (150 mL×2). The combined organic layers were washed with brine, dried over Na₂SO₄, filtered, concentrated to dryness. The resulting residue was purified by flash column chromatography to give the titled compound (13 g, 70%). ¹HNMR (400 MHz, CDCl₃) δ 8.83 (s, 1H), 8.60 (d, J=1.6 Hz, 1H), 8.28 (dd, J=1.6 Hz, 8.8 Hz, 1H), 8.13 (d, J=9.2 Hz, 1H), 2.82 (s, 3H), 2.76 (s, 3H) ppm. MS: M/e 187 (M+1)⁺.

### Step C: 1-(3-methylquinoxalin-6-yl)ethan-1-ol

To a solution of 1-(3-methylquinoxalin-6-yl)ethan-1-one (17.7 g, 95.16 mmol) in EtOH (170 ml) at 0° C. was added NaBH₄ (6.7 g, 0.17 mol) in some portions. After addition, the reaction mixture was stirred at 0° C. for 1 hour. The reaction mixture was quenched by water, extracted with DCM (300 ml, ×2). The combined organic layers were dried over Na₂SO₄, filtered and concentrated to dryness. The resulting residue was purified by flash column chromatography to give the titled compound (14 g, 79%). ¹H NMR (400 MHz, CDCl₃) δ 8.71 (s, 1H), 8.04 (d, J=8.8 Hz, 1H), 7.98 (d, J=0.8 Hz, 1H), 7.75 (dd, J=1.6 Hz, 8.4 Hz, 1H), 5.17-5.09 (m, 1H), 2.77 (s, 3H), 1.60 (d, J=6.8 Hz, 3H) ppm. MS: M/e 189 (M+1)⁺.

### Step D: 7-((2S,5R)-2,5-dimethyl-4-(1-(3-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of 1-(3-methylquinoxalin-6-yl)ethan-1-ol (200 mg, 1.1 mmol), Intermediate 10B (345 mg, 1 mmol) and DIPEA (645 mg, 5 mol) in CH₃CN (5 ml) was added (cyanomethyl)trimethylphosphonium iodide (729 mg. 3 mmol). The mixture was stirred at 105° C. in a sealed bottle for 16 hours. The reaction was cooled to room temperature, and then the solvent was removed under reduced pressure. The resulting residue was diluted by adding water, extracted with EA (80 mL×2), washed with brine, dried over Na₂SO₄, filtered and concentrated to dryness. The resulting residue was purified by flash column chromatography to give the titled compound (200 mg, 38%). MS: M/e 516 (M+1)⁺.

### Step E: 7-((2S,5R)-2,5-dimethyl-4-(1-(3-methylquinoxalin-6-yl)ethyl)piperazin-1-yl)-4-methyl-2,4-dihydro-5H-pyrazolo[4,3-b]pyridin-5-one

To a solution of 7-((2S,5R)-2,5-dimethyl-4-(1-(3-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (200 mg, 0.38 mmol) in MeOH (5 ml) was added a solution of 4M HCl in dioxane (2 mL). The mixture was stirred at room temperature for 4 hours. Another portion of 4M HCl in dioxane (2 mL) was added and the reaction was stirred at room temperature for 3 hours. The solvent was removed under reduced pressure. The resulting black residue (150 mg) was used directly for next step. MS: M/e 432 (M+1)⁺.

### Step F: 2-(7-((2S,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile

To a mixture of 7-((2S,5R)-2,5-dimethyl-4-(1-(3-methylquinoxalin-6-yl)ethyl)piperazin-1-yl)-4-methyl-2,4-dihydro-5H-pyrazolo[4,3-b]pyridin-5-one (150 mg, 0.34 mmol) and K₂CO₃ (524 mg, 3.8 mmol) in DMF (8 mL) was added 2-chloroacetonitrile (114 mg, 1.52 mmol). The reaction was stirred at room temperature overnight. The reaction was diluted with water, extracted with EA (60 mL×2), washed with brine, dried over Na₂SO₄, filtered and concentrated to dryness. The resulting residue was purified by prep-TLC (DCM/MeOH=15/1) to give the titled compound (30 mg, 16% for two steps), which was a mixture of diastereomers Compound A269c and Compound A269d. ¹HNMR (400 MHz, CD₃OD) δ 8.80-8.77 (m, 1H), 8.11-7.90 (m, 4H), 5.57 (s, 1H), 5.49-5.43 (m, 2H), 5.01-4.90 (m, 0.5H), 4.70-4.50 (m, 1H), 4.35-4.20 (m, 0.5H), 3.99-3.88 (m, 0.5H), 3.84-3.76 (m, 0.5H), 3.73-3.64 (m, 1H), 3.52-3.44 (m, 0.5H), 3.43 (s, 3H), 3.14-3.04 (m, 0.5H), 2.97-2.82 (m, 1.5H), 2.78-2.75 (m, 3H), 2.26-2.18 (m, 0.5H), 1.50-1.38 (m, 4.5H), 1.28-1.16 (m, 4H), 1.08-1.05 (m, 1.5H) ppm. MS: M/e 471 (M+1)⁺.

The Compound A269d can also be synthesized through another method as following:

### Compound A269d: 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile

### Step A: 7-bromo-2-methylquinoxaline

7-bromo-2-chloroquinoxaline (20 g, 82 mmol) and Ferric acetylacetonate (2.9 g, 8.2 mmol) were dissolved in dry THF (150 mL). A solution of methyl magnesium bromide (3M in ethyl ether) (30 ml, 91 mmol) was added dropwise at 0° C. After stirred for 1 hour, the reaction mixture was diluted with EA and quenched with 1M HCl aq·. The organic layer was separated, washed with water and brine and dried over Na₂SO₄, The organic solvent was removed under vacuum, The resulting residue was further purified by flash column chromatography (PE:EA=5:1) to give the titled compound (14.5 g, 79%). ¹H NMR (400 MHz, DMSO-d₆) δ 8.89 (s, 1H), 8.22 (d, J=2.0 Hz, 1H), 8.00 (d, J=8.9 Hz, 1H), 7.91 (dd, J=8.9, 2.0 Hz, 1H), 2.72 (s, 3H). MS: M/e 223 (M+1)⁺.

### Step B: 1-(3-methylquinoxalin-6-yl)ethan-1-one

To a solution of 7-bromo-2-methylquinoxaline (22 g, 0.1 mol) and Cs₂CO₃ (48.6 g, 0.15 mol) in DMF/H₂O (300 mL) was added 1-(vinyloxy)butane (80 g, 0.8 mol), Pd(OAc)₂ (1.8 g, 0.08 mol) and DPPP (9.88 g, 0.24 mol). The mixture was stirred at 116° C. under N₂ for 16 hours. The reaction mixture was cooled to room temperature, treated with 2N HCl (175 mL) and stirred for 1 hour. The reaction mixture was diluted with water, extracted with EA (150 mL×2). The combined organic layers were washed with brine, dried over Na₂SO₄, filtered, concentrated to dryness. The resulting residue was purified by flash column chromatography to give the titled compound (13 g, 70%). ¹HNMR (400 MHz, CDCl₃) δ 8.83 (s, 1H), 8.60 (d, J=1.6 Hz, 1H), 8.28 (dd, J=1.6 Hz, 8.8 Hz, 1H), 8.13 (d, J=9.2 Hz, 1H), 2.82 (s, 3H), 2.76 (s, 3H) ppm. MS: M/e 187 (M+1)⁺.

### Step C: 1-(3-methylquinoxalin-6-yl)ethan-1-ol

To a solution of 1-(3-methylquinoxalin-6-yl)ethan-1-one (17.7 g, 95.16 mmol) in EtOH (170 ml) at 0° C. was added NaBH₄ (6.7 g, 0.17 mol) in some portions. After addition, the reaction mixture was stirred at 0° C. for 1 hour. The reaction mixture was quenched by water, extracted with DCM (300 mL×2). The combined organic layers were dried over Na₂SO₄, filtered and concentrated to dryness. The resulting residue was purified by flash column chromatography to give the titled compound (14 g, 79%). ¹H NMR (400 MHz, CDCl₃) δ 8.71 (s, 1H), 8.04 (d, J=8.8 Hz, 1H), 7.98 (d, J=0.8 Hz, 1H), 7.75 (dd, J=1.6 Hz, 8.4 Hz, 1H), 5.17-5.09 (m, 1H), 2.77 (s, 3H), 1.60 (d, J=6.8 Hz, 3H) ppm. MS: M/e 189 (M+1)⁺.

### Step D: tert-butyl (2S,5R)-2,5-dimethyl-4-(1-(3-methylquinoxalin-6-yl)ethyl)piperazine-1-carboxylate

To a solution of 1-(3-methylquinoxalin-6-yl)ethan-1-ol (14 g, 74.4 mmol), tert-butyl (2S,5R)-2,5-dimethylpiperazine-1-carboxylate (20.7 g, 096.72 mmol) and DIPEA (37 g, 0.286 mol) in CH₃CN (200 ml) was added (cyanomethyl)trimethylphosphonium iodide (27 g. 111.6 mmol). The mixture was stirred at 105° C. in a sealed tube for 16 hours. The reaction mixture was cooled to room temperature, and then the reaction solvent was removed under reduced pressure. The resulting residue was redissolved into water. The resulting solution was extracted with EA (200 mL×3). The combined organic layers were washed with brine, dried over Na₂SO₄, filtered and concentrated to dryness. The resulting residue was purified by flash column chromatography to give the titled compound (24 g, 84%). MS: M/e 385 (M+1)⁺.

### Step E: 7-((S)-1-((2R,5S)-2,5-dimethylpiperazin-1-yl)ethyl)-2-methylquinoxaline, or 7-((R)-1-((2R,5S)-2,5-dimethylpiperazin-1-yl)ethyl)-2-methylquinoxaline

TFA (70 mL) was added to tert-butyl (2S,5R)-2,5-dimethyl-4-(1-(quinoxalin-6-yl)ethyl)piperazine-1-carboxylate (24.1 g) in DCM (200 ml). The mixture was stirred at room temperature for 16 hours. The reaction solvent was removed under reduced pressure. The resulting residue was diluted with water. The resulting aqueous layer was extracted with EA (400 mL), followed adjusting pH to 12-13 with saturated Na₂CO₃ aq·, and then extracted with DCM:IPA (4:1, 200 mL×5). The combined organic layers were dried over Na₂SO₄, filtered, and concentrated to dryness. The resulting residue (18 g) as a mixture of diastereomers was performed chiral resolution by C18 column (mobile phase B: MeOH mobile phase A: H₂O (0.5% NH₃H₂O)) to give the titled compounds 7-((S)-1-((2R,5S)-2,5-dimethylpiperazin-1-yl)ethyl)-2-methylquinoxaline (the earlier peak, a single diastereoisomer, 6.3 g, 35%) and 7-((R)-1-((2R,5S)-2,5-dimethylpiperazin-1-yl)ethyl)-2-methylquinoxalin (the latter peak, a single diastereoisomer, 7.3 g, 40%). 7-((S)-1-((2R,5S)-2,5-dimethylpiperazin-1-yl)ethyl)-2-methylquinoxaline (the earlier peak): ¹HNMR (400 MHz, DMSO-d₆) δ 8.82 (s, 1H), 8.00 (d, J=8.8 Hz, 1H), 7.81 (d, J=1.6 Hz, 1H), 7.71 (dd, J=2.0 Hz, 8.4 Hz, 1H), 4.47-4.37 (m, 1H), 2.95 (dd, J=2.0, 10.4 Hz, 1H), 2.70 (s, 3H), 2.69-2.57 (m, 2H), 2.37-2.29 (m, 1H), 2.07-1.98 (m, 1H), 1.49 (d, J=6.8 Hz, 3H), 1.45-1.36 (m, 1H), 1.08 (d, J=6.0 Hz, 3H), 0.86 (d, J=6.4 Hz, 3H) ppm. MS: M/e 285 (M+1)⁺.

7-((R)-1-((2R,5S)-2,5-dimethylpiperazin-1-yl)ethyl)-2-methylquinoxaline (the later peak): ¹HNMR (400 MHz, DMSO-d₆) δ 8.81 (s, 1H), 7.99 (d, J=9.2 Hz, 1H), 7.89 (d, J=7.3 Hz, 2H), 4.44 (q, J=6.5 Hz, 1H), 3.96 (d, J=41.3 Hz, 1H), 2.92 (dd, J=11.5, 1.9 Hz, 1H), 2.70 (s, 3H), 2.65-2.54 (m, 2H), 2.48 (s, 1H), 2.14 (dd, J=11.1, 2.1 Hz, 1H), 1.89 (t, J=10.6 Hz, 1H), 1.36 (t, J=12.2 Hz, 3H), 1.13 (d, J=6.1 Hz, 3H), 0.80 (d, J=6.3 Hz, 3H) ppm. MS: M/e 285 (M+1)⁺.

### Step F: 7-((2S,5R)-2,5-dimethyl-4-((S)-1-(3-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one

To a solution of 7-((S)-1-((2R,5S)-2,5-dimethylpiperazin-1-yl)ethyl)-2-methylquinoxaline (4.0 g, 14.0 mmol) and Intermediate 2 (6.4 g, 16.8 mmol) in 1,4-dioxane (30 mL) was added DIPEA (4.3 g, 33.5 mmol). Then the reaction mixture was heated at 100° C. in a sealed tube for 16 hours. The reaction mixture was cooled to room temperature, concentrated to dryness. The resulting residue was diluted with water. The resulting solution was extracted with EA (100 mL×3). The combined organic layers were washed with brine, dried over Na₂SO₄, filtered, concentrated to dryness. The resulting residue was purified by flash column chromatography to give the titled compound (5.6 g, 77%). MS: M/e 516 (M+1)⁺.

### Step G: 7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one

A solution of 7-((2S,5R)-2,5-dimethyl-4-((S)-1-(3-methylquinoxalin-6-yl)ethyl)piperazin-1-yl)-4-methyl-2-(tetrahydro-2H-pyran-2-yl)-2,4-dihydro-5H-pyrazolo[4,3-b]pyridin-5-one (0.4 g, 0.77 mmol) in TFA (10 mL) was stirred at room temperature overnight. The mixture was concentrated under reduced pressure, diluted with water/DCM, basified with saturated NaHCO₃ solution to pH 7-8, extracted with DCM:IPA (4:1, 60 mL×4). The combined organic layers were dried over Na₂SO₄, filtered, concentrated to dryness. The resulting residue was purified by flash column chromatography to give the titled compound (0.18 g, 54%). MS: M/e 432 (M+1)⁺.

### Step H: 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile

To a mixture of 7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one (180 mg, 0.42 mmol) and K₂CO₃ (173 mg, 1.26 mmol) in DMF (5 mL) was added 2-chloroacetonitrile (63 mg, 0.84 mmol). The reaction was stirred at room temperature overnight. The reaction mixture was diluted with water, extracted with EA (80 mL×3), washed with brine, dried over Na₂SO₄, filtered, and concentrated to dryness. The resulting residue was purified by flash column chromatography to give titled compound (115 mg, 76%). ¹HNMR (400 MHz, CD₃OD) δ 8.79 (s, 1H), 8.06 (d, J=8.8 Hz, 1H), 7.99-7.90 (m, 3H), 5.57 (s, 1H), 5.46 (s, 2H), 5.01-4.88 (m, 1H), 4.36-4.20 (m, 1H), 3.99-3.90 (m, 1H), 3.50-3.43 (m, 1H), 3.43 (s, 3H), 3.12-3.04 (m, 1H), 2.97-2.86 (m, 2H), 2.77 (s, 3H), 1.48-1.39 (m, 6H), 1.05 (d, J=6.4 Hz, 3H) ppm. MS: M/e 471 (M+1)⁺.

Compound A269c was also prepared according to the similar procedures as described above for Compound A269d under appropriate conditions using 7-((R)-1-((2R,5S)-2,5-dimethylpiperazin-1-yl)ethyl)-2-methylquinoxaline as a start material, which could be recognized by one skilled in the art.

Compound A269c: ¹HNMR (400 MHz, CD₃OD) δ 8.77 (s, 1H), 8.08-7.84 (m, 4H), 5.56 (s, 1H), 5.48 (s, 2H), 4.60 (s, 2H), 3.79 (d, J=4.7 Hz, 1H), 3.68 (d, J=10.4 Hz, 2H), 3.43 (s, 3H), 2.83 (d, J=10.4 Hz, 1H), 2.76 (s, 3H), 2.20 (d, J=11.7 Hz, 1H), 1.45 (d, J=6.3 Hz, 3H), 1.21 (dd, J=11.8, 6.4 Hz, 6H) ppm. MS: M/e 471 (M+1)⁺.

Compound A270: 2-(7-((2S,5R)-4-(1-(3-methoxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 7-bromo-2-methoxyquinoxaline

To a stirred solution of 7-bromo-2-chloroquinoxaline (4.86 g, 20 mmol) in MeOH (50 mL) was added K₂CO₃ (5.52 g, 40 mmol). After the addition, the reaction mixture was stirred at 70° C. overnight. The reaction mixture was concentrated to dryness. The resulting residue was purified by flash column chromatography to give the titled compound (4.8 g). MS: M/e 239/241 (M+1)⁺.

### Step B: ethyl 3-methoxyquinoxaline-6-carboxylate

A mixture of 7-bromo-2-methoxyquinoxaline (3.8 g, 16 mmol), CH₃COONa (5.2 g, 64 mmol), PddppfCl₂ (580 mg, 0.8 mmol) in DMF/EtOH (4 mL/20 mL) was stirred at 90° C. overnight under CO (2 MPa) overnight. The reaction mixture was allowed cool to RT. A suspension was formed. The filter cake was collected by filtration and dried to give the titled compound (crude), which was used to the next step directly. MS: M/e 233 (M+1)⁺.

### Step C: 3-methoxyquinoxaline-6-carboxylic acid

To a stirred solution of ethyl 3-methoxyquinoxaline-6-carboxylate (crude, 20 mmol) in MeOH (100 mL) was added a solution of NaOH (1.6 g, 40 mmol) in H₂O (10 mL). After the addition, the reaction mixture was stirred overnight. The solvent MeOH was removed. The resulting aqueous layer was acidified to pH=3-4 with HCl aq. to give a suspension. The filter cake was collected by filtration and dried to give the titled compound (2.9 g, 71%). MS: M/e 205 (M+1)⁺.

### Step D: N,3-dimethoxy-N-methylquinoxaline-6-carboxamide

A mixture of 3-methoxyquinoxaline-6-carboxylic acid (2.9 g, 14.2 mmol), N,O-dimethylhydroxylamine hydrochloride (1.66 g, 17.1 mmol), HATU (6.5 g, 17.1 mmol) and DIPEA (3.7 g, 28.4 mmol) in DMF (20 mL) was stirred at RT overnight. The reaction mixture was poured into H₂O (50 mL) and extracted with EA (20 mL×4). The combined organic layers were washed with brine, dried over Na₂SO₄, concentrated to dryness. The resulting residue was purified by flash column chromatography to give the titled compound (1.8 g, 51%). MS: M/e 248 (M+1)⁺.

### Step E: 1-(3-methoxyquinoxalin-6-yl)ethan-1-one

To a stirred solution of N,3-dimethoxy-N-methylquinoxaline-6-carboxamide (0.99 g, 4 mmol) in THF (10 mL) was added dropwise MeMgBr (3.0 M, 1.47 mL, 4.4 mmol) at 0° C. After the addition, the reaction was stirred for an hour. The reaction mixture was quenched with NH₄Cl aq. and extracted with EA (20 mL×3). The combined organic layers were washed with brine, dried over Na₂SO₄, concentrated to give the crude product, which was further purified by flash column chromatography to give the titled compound (670 mg, 83%). MS: M/e 203 (M+1)⁺.

### Step F: 1-(3-methoxyquinoxalin-6-yl)ethan-1-ol

To a stirred solution of 1-(3-methoxyquinoxalin-6-yl)ethan-1-one (670 mg, 3.3 mmol) in EtOH (5 mL) was added NaBH₄ (100 mg, 2.65 mmol). After then, the reaction was stirred for 20 minutes. The reaction mixture was poured into H₂O (30 mL) and extracted with EA (10 mL×2). The combined organic layers were washed with brine, dried over Na₂SO₄, concentrated to give the titled compound (667 mg, 99%). MS: M/e 205 (M+1)⁺.

### Step G: 7-((2S,5R)-4-(1-(3-methoxyquinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

A mixture of Intermediate 10B (69 mg, 0.2 mmol), 1-(3-methoxyquinoxalin-6-yl)ethan-1-ol (81.6 mg, 0.4 mmol), (cyanomethyl)trimethylphosphonium iodide (146 mg, 0.6 mmol) and DIPEA (258 mg, 2 mmol) in CH₃CN (4 mL) was stirred at 100° C. overnight in a sealed tube. The reaction mixture was diluted with EA (15 mL), washed with H₂O, brine, dried over Na₂SO₄, concentrated to dryness. The resulting residue was purified by Pre-TLC (EA) to give the titled compound (61 mg, 57%). MS: M/e 532 (M+1)⁺.

### Step H: 2-(7-((2S,5R)-4-(1-(3-methoxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a stirred solution of 7-((2S,5R)-4-(1-(3-methoxyquinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (61 mg, 0.11 mmol) in MeOH (5 mL) was added HCl (2 mL, 4.0 M, in 1,4-dioxane). Then the reaction was stirred overnight. The reaction mixture was concentrated to give the residue, which was treated with H₂O, basified to pH=9-10 with Na₂CO₃ aq·, extracted with EA (10 mL×2). The combined organic layers were washed with brine, dried over Na₂SO₄, concentrated to give 7-((2S,5R)-4-(1-(3-methoxyquinoxalin-6-yl)ethyl)-2,5-dimethylpiperazin-1-yl)-4-methyl-2,4-dihydro-5H-pyrazolo[4,3-b]pyridin-5-one as a mixture. The above product was dissolved in DMF (5 mL). To the resulting solution was added K₂CO₃ (30.1 mg, 0.32 mmol), followed by 2-chloroacetonitrile (8.6 mg, 0.22 mmol). Then the reaction was stirred for 2 days. The reaction mixture was poured into H₂O (15 mL) and extracted with EA (10 mL×2). The combined organic layers were washed with brine, dried over Na₂SO₄ and concentrated to dryness. The resulting residue was purified by Pre-TLC (CH₂Cl₂:MeOH=10:1) to give the titled compound (16 mg). ¹H NMR (400 MHz, CD₃OD) δ 8.43 (d, J=5.2 Hz, 1H), 8.01-7.91 (m, 2H), 7.86 (d, J=6.4 Hz, 1H), 7.75 (d, J=8.4 Hz, 1H), 5.56 (s, 1H), 5.47 (d, J=4.0 Hz, 2H), 4.70-4.22 (m, 2H), 4.11 (d, J=5.6 Hz, 3H), 3.98-3.73 (m, 1H), 3.73-3.64 (m, 1H), 3.47 (d, J=3.2 Hz, 0.5H), 3.43 (s, 3H), 3.15-2.73 (m, 2H), 2.31-2.18 (m, 0.5H), 1.49-1.40 (m, 4.5H), 1.22 (dd, J=10.8, 6.4 Hz, 3H), 1.05 (d, J=6.4 Hz, 1.5H) ppm. MS: M/e 487 (M+1)⁺.

### Compound A271: 2-(7-((2S,5R)-4-(1-(4-fluoro-2-(3-hydroxyoxetan-3-yl)phenyl)ethyl)-2,5-dimethylpiperazin-1-yl)-4-methyl-5-oxo-4,5-dihydro-2H-pyrazolo[4,3-b]pyridin-2-yl)acetonitrile

### Step A: tert-butyl (2S,5R)-4-(1-(4-fluoro-2-(3-hydroxyoxetan-3-yl)phenyl)ethyl)-2,5-dimethylpiperazine-1-carboxylate

To a solution of tert-butyl (2S,5R)-4-(1-(2-bromo-4-fluorophenyl)ethyl)-2,5-dimethylpiperazine-1-carboxylate (100 mg, 0.24 mmol) in THF (3 mL) was added n-BuLi (0.3 mL, 0.48 mmol) at −78° C. After 1 hour, a solution of oxetan-3-one (18 mg, 0.25 mmol) was added. The reaction was kept at −78° C. for 2 hours. The reaction mixture quenched with saturated NH₄Cl aq·, extracted with EA (60 mL×2). The combined organic layers were washed with brine, dried over Na₂SO₄, filtered, and concentrated. The residue was purified by flash column chromatography (EA:PE=2:1) to give titled compound (90 mg, 91%). MS: M/e 409 (M+1)⁺.

### Step B: 3-(2-(1-((2R,5S)-2,5-dimethylpiperazin-1-yl)ethyl)-5-fluorophenyl)oxetan-3-ol

To a solution of tert-butyl (2S,5R)-4-(1-(4-fluoro-2-(3-hydroxyoxetan-3-yl)phenyl)ethyl)-2,5-dimethylpiperazine-1-carboxylate (90 mg, 0.22 mmol) in DCM (5 mL) was added TFA (1 mL). The reaction was stirred at room temperature for 16 hours. The reaction mixture was concentrated to dryness. The resulting residue (70 mg) was used to next step directly without further purification. MS: M/e 309 (M+1)⁺.

### Step C: 7-((2S,5R)-4-(1-(4-fluoro-2-(3-hydroxyoxetan-3-yl)phenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

To a solution of 3-(2-(1-((2R,5S)-2,5-dimethylpiperazin-1-yl)ethyl)-5-fluorophenyl)oxetan-3-ol (70 mg, crude) and Intermediate 2 (70 mg, 0.2 mmol) in CH₃CN (3 mL) was added DIEA (258 mg, 2 mmol). Then the mixture was heated at 90° C. for 16 hours. The reaction mixture was cooled to RT, quenched with water and extracted with EA (60 mL×3). The combined organic layers were washed with brine, dried over Na₂SO₄, filtered concentrated to dryness. The resulting residue was purified by flash column chromatography (DCM:MeOH=15:1) to give the titled compound (12 mg). MS: M/e 540 (M+1)⁺.

### Step D: 7-((2S,5R)-4-(1-(4-fluoro-2-(3-hydroxyoxetan-3-yl)phenyl)ethyl)-2,5-dimethylpiperazin-1-yl)-4-methyl-2,4-dihydro-5H-pyrazolo[4,3-b]pyridin-5-one

To a solution of 7-((2S,5R)-4-(1-(4-fluoro-2-(3-hydroxyoxetan-3-yl)phenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (12 mg, 0.02 mmol) in DCM (1 mL) was added TFA (5 mL). The resulted mixture was stirred at RT overnight. The reaction mixture was concentrated to dryness. The resulting residue (12 mg) was used to next step directly without further purification. MS: M/e 456 (M+1)⁺.

### Step F: 2-(7-((2S,5R)-4-(1-(4-fluoro-2-(3-hydroxyoxetan-3-yl)phenyl)ethyl)-2,5-dimethylpiperazin-1-yl)-4-methyl-5-oxo-4,5-dihydro-2H-pyrazolo[4,3-b]pyridin-2-yl)acetonitrile

To a mixture of 7-((2S,5R)-4-(1-(4-fluoro-2-(3-hydroxyoxetan-3-yl)phenyl)ethyl)-2,5-dimethylpiperazin-1-yl)-4-methyl-2,4-dihydro-5H-pyrazolo[4,3-b]pyridin-5-one (12 mg, 0.0222 mmol) and K₂CO₃ (30 mg, 0.2 mmol) in DMF (2 mL) was added 2-chloroacetonitrile (10 mg, 0.12 mmol). The reaction was stirred at RT overnight. The reaction mixture was diluted with water and extracted with EA (40 mL×2). The combined organic layers were washed with brine, dried over Na₂SO₄, filtered concentrated to dryness. The resulting residue was purified by Prep-TLC (DCM:MeOH=15:1) to give the titled compound (4 mg, 36% for 2 steps). ¹HNMR (400 MHz, CD₃OD) δ 7.94-7.90 (m, 1H), 7.85-7.65 (m, 1H), 7.16-6.90 (m, 2H), 5.58-5.54 (m, 1H), 5.49-5.44 (m, 2H), 5.22-5.14 (m, 1H), 5.08-5.02 (0.5H), 4.84-4.72 (m, 2H), 4.65-4.40 (m, 1.5H), 3.85-3.50 (m, 3H), 3.45-3.41 (m, 3H), 3.08-2.92 (m, 1.5H), 2.86-2.72 (m, 1H), 2.25-2.15 (m, 0.5H), 1.46-1.29 (m, 5H), 1.21-1.12 (m, 4H) ppm. MS: M/e 495 (M+1)⁺.

### Compound A274: 2-(7-((2S,5R)-2,5-dimethyl-4-((S)-1-(quinoxalin-6-yl)ethyl)piperazin-1-yl)-6-methoxy-4-methyl-5-oxo-4,5-dihydro-2H-pyrazolo[4,3-b]pyridin-2-yl)acetonitrile

### Step A: 7-((2S,5R)-2,5-dimethyl-4-((S)-1-(quinoxalin-6-yl)ethyl)piperazin-1-yl)-6-hydroxy-4-methyl-2-(tetrahydro-2H-pyran-2-yl)-2,4-dihydro-5H-pyrazolo[4,3-b]pyridin-5-one

To a solution of 2-(6-bromo-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile (247 mg, 0.514 mmol)(obtained via bromination of Compound A2a using NBS, as a single diastereoisomer), Pd₂(dba)₃ (24 mg, 0.026 mmol) and tBuXPhos (22 mg, 0.051 mmol) in 1,4-dioxane (5 mL) was added KOH (0.51 mL, 1.54 mmol, 3M). The reaction mixture was degassed 3 times under N₂ atmosphere and stirred at 90° C. for 12 hours. The reaction mixture was quenched with saturated NH₄Cl aq·(10 mL) and extracted with EA (30 mL×2). The combined organic layers were dried over Na₂SO₄ and concentrated to dryness. The resulting residue was purified by Prep-TLC to give the titled compound (25 mg, 9%). MS: M/e 518 (M+1)⁺.

### Step B: 7-((2S,5R)-2,5-dimethyl-4-((S)-1-(quinoxalin-6-yl)ethyl)piperazin-1-yl)-6-methoxy-4-methyl-2-(tetrahydro-2H-pyran-2-yl)-2,4-dihydro-5H-pyrazolo[4,3-b]pyridin-5-one

To a solution of 7-((2S,5R)-2,5-dimethyl-4-((S)-1-(quinoxalin-6-yl)ethyl)piperazin-1-yl)-6-hydroxy-4-methyl-2-(tetrahydro-2H-pyran-2-yl)-2,4-dihydro-5H-pyrazolo[4,3-b]pyridin-5-one (25 mg, 0.05 mmol) and Cs₂CO₃ (32 mg, 0.10 mmol) in DMF (2 mL) was added CH₃I (14 mg, 0.10 mmol). Then the reaction mixture was stirred at RT for 4 hours. The reaction mixture was quenched with saturated NH₄Cl aq·(2 mL) and extracted with EA (10 mL×2). The combined organic layers were dried over Na₂SO₄ and concentrated to dryness. The resulting residue was purified by Prep-TLC to give the titled compound (15 mg, 59%). MS: M/e 532 (M+1)⁺.

### Step C: 7-((2S,5R)-2,5-dimethyl-4-((S)-1-(quinoxalin-6-yl)ethyl)piperazin-1-yl)-6-methoxy-4-methyl-2,4-dihydro-5H-pyrazolo[4,3-b]pyridin-5-one

To a solution of 7-((2S,5R)-2,5-dimethyl-4-((S)-1-(quinoxalin-6-yl)ethyl)piperazin-1-yl)-6-methoxy-4-methyl-2-(tetrahydro-2H-pyran-2-yl)-2,4-dihydro-5H-pyrazolo[4,3-b]pyridin-5-one (15 mg, 0.028 mmol) in DCM (3 mL) was added HCl (1 mL, 4 M in 1,4-dioxane). The mixture was stirred at RT for 1 hour. Then the reaction solvent was removed to give the crude product as a HCl salt. The resulting crude product was dissolved into water. The aqueous was basified to pH=10 with Na₂CO₃ aq·(4M) and extracted with EA (20 mL×3). The combined organic layers were washed with brine, dried over Na₂SO₄ and concentrated to give the titled compound (10 mg, 79%). MS: M/e 448 (M+1)⁺.

### Step D: 2-(7-((2S,5R)-2,5-dimethyl-4-((S)-1-(quinoxalin-6-yl)ethyl)piperazin-1-yl)-6-methoxy-4-methyl-5-oxo-4,5-dihydro-2H-pyrazolo[4,3-b]pyridin-2-yl)acetonitrile

To a solution of 7-((2S,5R)-2,5-dimethyl-4-((S)-1-(quinoxalin-6-yl)ethyl)piperazin-1-yl)-6-methoxy-4-methyl-2,4-dihydro-5H-pyrazolo[4,3-b]pyridin-5-one (10 mg, 0.022 mmol) and K₂CO₃ (6 mg, 0.044 mmol) in DMF (3 mL) was added 2-iodoacetonitrile (6 mg, 0.033 mmol). The reaction was stirred at RT for 6 hours. Then the reaction mixture was quenched with saturated NaCl (10 mL) and extracted with EA (30 mL×2). The combined organic layers were dried over Na₂SO₄ and concentrated under reduced pressure. The residue was purified by Prep-HPLC (Method A) to give the titled compound (4 mg) as a single diastereoisomer. ¹H NMR (400 MHz, CD₃OD) δ 8.90 (d, J=3.5 Hz, 2H), 8.15 (d, J=9.2 Hz, 2H), 8.05 (dd, J=8.7, 1.5 Hz, 1H), 7.92 (s, 1H), 5.44 (s, 2H), 4.70-4.64 (m, 1H), 4.33-4.28 (m, 1H), 3.83-3.76 (m, 1H), 3.73 (s, 3H), 3.49 (s, 3H), 3.38-3.32 (m, 2H), 2.87-2.83 (m, 1H), 2.73-2.68 (m, 1H), 1.56 (d, J=6.7 Hz, 3H), 1.29 (d, J=6.4 Hz, 3H), 1.15 (d, J=6.3 Hz, 3H) ppm. MS: M/e 487 (M+1)⁺.

### Compound A276: 2-(7-((2S,5R)-2,5-diethyl-4-(1-(4-fluoro-2-(trifluoromethyl)phenyl)ethyl)piperazin-1-yl)-4-(methyl-d3)-5-oxo-4,5-dihydro-2H-pyrazolo[4,3-b]pyridin-2-yl)acetonitrile

### Step A: 7-((2S,5R)-2,5-diethyl-4-(1-(4-fluoro-2-(trifluoromethyl)phenyl)ethyl)piperazin-1-yl)-4-(methyl-d3)-2-(tetrahydro-2H-pyran-2-yl)-2,4-dihydro-5H-pyrazolo[4,3-b]pyridin-5-one

To a solution of 4-(methyl-d3)-5-oxo-2-(tetrahydro-2H-pyran-2-yl)-4,5-dihydro-2H-pyrazolo[4,3-b]pyridin-7-yl trifluoromethanesulfonate (115 mg, 0.3 mmol) and (2R,5S)-2,5-diethyl-1-(1-(4-fluoro-2-(trifluoromethyl)phenyl)ethyl)piperazine (119 mg, 0.36 mmol) in CH₃CN (2 mL) was added DIPEA (78 mg, 0.62 mmol). Then the reaction mixture was heated at 90° C. under N₂ for 16 hours. The reaction mixture was cooled to RT, diluted with water (50 mL) and extracted with EA (60 mL×2). The combined organic layers were washed with brine, dried over Na₂SO₄, filtered concentrated to dryness. The resulting residue was purified by flash column chromatography (DCM:MeOH=15:1) to give the titled compound (110 mg, 64%). MS: M/e 567 (M+1)⁺.

### Step B: 7-((2S,5R)-2,5-diethyl-4-(1-(4-fluoro-2-(trifluoromethyl)phenyl)ethyl)piperazin-1-yl)-4-(methyl-d3)-2,4-dihydro-5H-pyrazolo[4,3-b]pyridin-5-one

To a solution of 7-((2S,5R)-2,5-diethyl-4-(1-(4-fluoro-2-(trifluoromethyl)phenyl)ethyl)piperazin-1-yl)-4-(methyl-d3)-2-(tetrahydro-2H-pyran-2-yl)-2,4-dihydro-5H-pyrazolo[4,3-b]pyridin-5-one (110 mg, 0.19 mmol) in DCM (1 mL) was added TFA (5 mL). The resulting mixture was stirred at RT overnight. The reaction mixture was concentrated under reduced pressure to give the titled compound (110 mg, crude). MS: M/e 483 (M+1)⁺.

### Step C: 2-(7-((2S,5R)-2,5-diethyl-4-(1-(4-fluoro-2-(trifluoromethyl)phenyl)ethyl)piperazin-1-yl)-4-(methyl-d3)-5-oxo-4,5-dihydro-2H-pyrazolo[4,3-b]pyridin-2-yl)acetonitrile

To a solution of 7-((2S,5R)-2,5-diethyl-4-(1-(4-fluoro-2-(trifluoromethyl)phenyl)ethyl)piperazin-1-yl)-4-(methyl-d3)-2,4-dihydro-5H-pyrazolo[4,3-b]pyridin-5-one (110 mg, crude) and K₂CO₃ (276 mg, 2 mmol) in DMF (3 mL) was added 2-chloroacetonitrile (28 mg, 0.38 mmol). The reaction was stirred at RT overnight. The reaction mixture was diluted with water, extracted with EA (60 mL×2). The combined organic layers were washed with brine, dried over Na₂SO₄, filtered and concentrated to dryness. The resulting residue was purified by prep-TLC (DCM:MeOH=15:1) to give the titled compound (24 mg, 24% for two steps). ¹HNMR (400 MHz, CD₃OD) δ 8.10-8.00 (m, 1H), 7.92 (s, 1H), 7.47-7.35 (m, 2H), 5.57 (s, 1H), 5.47 (s, 2H), 4.23-4.14 (m, 0.5H), 4.08-3.98 (m, 0.5H), 3.55-3.44 (m, 0.5H), 3.31-3.28 (m, 2H), 3.24-3.16 (m, 0.5H), 3.13-3.06 (m, 0.5H), 2.96-2.82 (m, 0.5H), 2.74-2.62 (m, 0.5H), 2.35-2.26 (m, 0.5H), 2.24-2.12 (m, 1H), 1.98-1.80 (m, 1H), 1.76-1.45 (m, 3H), 1.29 (t, J=6.4 Hz, 3H), 1.11-0.91 (m, 3H), 0.73 (t, J=6.4 Hz, 1.5H), 0.58 (t, J=6.4 Hz, 1.5H) ppm. MS: M/e 522 (M+1)⁺.

### Compound A278: 2-(7-((2S,5R)-4-(1-(3-ethylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 7-bromo-2-ethylquinoxaline

To a solution of 7-bromo-2-chloroquinoxaline (1.21 g, 5 mmol) and Iron(III) acetylacetonate (175 mg, 0.5 mmol) in dry THF (15 ml) was added EtMgBr (1M in THF, 7 ml) at 0° C. The resulting mixture was stirred at 0° C. for 1 hour. The reaction mixture was quenched with HCl aq·(1 M) and extracted with EA (25 mL×2). The organic layer was washed with brine, dried over Na₂SO₄, filtered and concentrated to dryness. The resulting residue was purified by flash column chromatography (EA/PE) to give the titled compound (1 g, 85%). MS: M/e 237 (M+1)⁺.

### Step B: 1-(3-ethylquinoxalin-6-yl)ethan-1-one

To a solution of 7-bromo-2-ethylquinoxaline (1 g, 4.2 mmol) in toluene (15 mL) was added 1-Ethoxy-1-(tributylstannyl)ethene (2 g, 5.5 mmol) and Dichlorobis(triphenylphosphine)palladium (294 mg, 0.42 mmol). The resulting mixture was stirred at 90° C. overnight. The reaction mixture was cooled to room temperature. Then to the above mixture, HCl aq·(4 N in 1,4-dioxane, 20 mL) was added. The resulting mixture was stirred at RT for 0.5 hour and extracted with EA (50 mL×2). The combined organic layers were washed with saturated sodium bicarbonate and brine, dried over Na₂SO₄ and concentrated to dryness. The resulting residue was purified by flash column chromatography (EA/hexanes) to give the titled compound (120 mg, 14%). MS: M/e 201 (M+1)⁺.

### Step C: 1-(3-ethylquinoxalin-6-yl)ethan-1-ol

To a solution of 1-(3-ethylquinoxalin-6-yl)ethan-1-one (120 mg, 0.6 mmol) in methanol (4 mL) was added sodium borohydride (18 mg, 0.48 mmol). The reaction mixture was stirred at 0° C. for 1 hour. The solvent was removed under reduced pressure. The resulting residue was dissolved into DCM and water. The organic layers were washed with brine, dried over Na₂SO₄ and concentrated to give the titled compound (120 mg), which was used to the next step directly without further purification. MS: M/e 203 (M+1)⁺.

### Step D: 7-((2S,5R)-4-(1-(3-ethylquinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

A sealed tube was charged with 1-(3-ethylquinoxalin-6-yl)ethan-1-ol (120 mg, 0.59 mmol), Intermediate 10B (136 mg, 0.4 mmol), (cyanomethyl)trimethylphosphonium iodide (291 mg, 1.2 mmol), DIEA (260 mg, 2 mmol) and acetonitrile (5 ml). The mixture was stirred at 100° C. overnight, then cooled to RT. The reaction was quenched with water and the aqueous was extracted with EA (25 mL×2). The combined organic layers were washed with brine, dried over Na₂SO₄ and concentrated to dryness. The resulting residue was purified by flash column chromatography (MeOH/DCM) to give the titled compound (60 mg, 28%). MS: M/e 530 (M+1)⁺.

### Step E: 7-((2S,5R)-4-(1-(3-ethylquinoxalin-6-yl)ethyl)-2,5-dimethylpiperazin-1-yl)-4-methyl-2,4-dihydro-5H-pyrazolo[4,3-b]pyridin-5-one

To a mixture of 7-((2S,5R)-4-(1-(3-ethylquinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (60 mg) in MeOH (2 mL) was added HCl (4 M in 1,4-dioxane, 2 ml). The reaction was stirred at RT for 2 hours. The reaction solvent was removed under reduced pressure. The resulting residue (50 mg) was used directly to next step without further purification. MS: M/e 446 (M+1)⁺.

### Step F: 2-(7-((2S,5R)-4-(1-(3-ethylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a mixture of 7-((2S,5R)-4-(1-(3-ethylquinoxalin-6-yl)ethyl)-2,5-dimethylpiperazin-1-yl)-4-methyl-2,4-dihydro-5H-pyrazolo[4,3-b]pyridin-5-one (50 mg, 0.11 mmol) in DMF (3 mL) was added potassium carbonate (77 mg, 0.56 mmol) and 2-iodoacetonitrile (38 mg, 0.22 mmol). The resulting mixture was stirred at room temperature overnight. The reaction mixture was quenched with water and extracted with EA. The organic layer was washed with brine, dried over Na₂SO₄, filtered and concentrated. The resulting residue was purified by prep-TLC(DCM:MeOH=13:1) to give the titled compound (7 mg, 13%). ¹H NMR (400 MHz, DMSO) δ 8.92 (d, J=4.5 Hz, 1H), 8.11 (dd, J=12.1, 8.6 Hz, 1H), 8.05 (s, 1H), 8.02 (d, J=12.7 Hz, 1H), 7.94 (t, J=6.5 Hz, 1H), 5.68 (s, 2H), 5.47 (d, J=5.9 Hz, 1H), 3.90 (dd, J=54.2, 6.3 Hz, 1H), 3.60 (d, J=12.6 Hz, 1H), 3.34 (s, 4H), 3.14-3.06 (m, 2H), 3.03 (d, J=14.4 Hz, 1H), 2.89 (s, 1H), 2.83-2.71 (m, 1H), 2.26-2.11 (m, 1H), 1.42 (dt, J=7.5, 3.7 Hz, 4H), 1.37 (d, J=6.5 Hz, 1H), 1.30 (s, 3H), 1.17 (t, J=6.1 Hz, 3H), 1.03 (d, J=6.5 Hz, 1H) ppm. MS: M/e 485 (M+1)⁺.

### Compound A279: 2-(7-((2S,5R)-4-(1-(3-hydroxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a stirred solution of 2-(7-((2S,5R)-4-(1-(3-methoxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile (81 mg, 0.172 mmol) in dioxane (8 mL) was added HCl aq·(2.0 M, 2 mL). After the addition, the reaction mixture was stirred at 80° C. for 3 hours. The reaction mixture was basified to pH=9-10 with NaHCO₃ aq·, extracted with EA (10 mL×3). The combined organic layers were washed with brine, dried over Na₂SO₄, concentrated to dryness. The resulting residue was purified by Pre-TLC (CH₂Cl₂:MeOH=10:1) to give the titled compound (20 mg, 17%). ¹H NMR (400 MHz, CD₃OD) δ 8.16 (d, J=3.6 Hz, 1H), 7.93 (s, 1H), 7.83-7.76 (m, 1H), 7.49-7.41 (m, 2H), 5.57 (s, 1H), 5.47 (d, J=2.4 Hz, 2H), 3.84-3.73 (m, 1H), 3.69-3.58 (m, 2H), 3.50-3.46 (m, 1H), 3.44 (s, 3H), 3.07-2.79 (m, 3H), 2.21 (d, J=11.4 Hz, 1H), 1.46-1.34 (m, 4H), 1.25 (d, J=6.4 Hz, 2H), 1.17 (d, J=6.4 Hz, 2H), 1.03 (d, J=6.4 Hz, 1H) ppm. MS: M/e 473 (M+1)⁺.

### Compound A280: 2-(7-((2S,5R)-4-(1-(3-chloroquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

A mixture of 2-(7-((2S,5R)-4-(1-(3-hydroxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile (8 mg, 0.017 mmol) in POCl₃ (0.5 mL) was stirred at 80° C. for 2 hours. The reaction mixture was quenched with NaHCO₃ aq·. The resulting mixture was extracted with EA (10 mL×2). The combined organic layers were washed with brine, dried over Na₂SO₄ and concentrated to dryness. The resulting residue was purified by Prep-HPLC (Method A) to give the titled compound (1.5 mg). ¹H NMR (400 MHz, CD₃OD) δ 8.83 (d, J=4.4 Hz, 1H), 8.15-8.08 (m, 1H), 8.05-7.98 (m, 2H), 7.92 (d, J=1.2 Hz, 1H), 5.57 (s, 1H), 5.46 (d, J=3.2 Hz, 2H), 4.69-4.50 (m, 2H), 4.01-3.79 (m, 1H), 3.73-3.65 (m, 1H), 3.51-3.44 (m, 0.5H), 3.44 (s, 3H), 3.14-3.05 (m, 0.5H), 2.96-2.84 (m, 1.5H), 2.20 (d, J=12.4 Hz, 0.5H), 1.48-1.41 (m, 4H), 1.26-1.18 (m, 4H), 1.06 (d, J=6.4 Hz, 1H) ppm. MS: M/e 491 (M+1)⁺.

### Compound A281: 2-(7-((2S,5R)-2,5-diethyl-4-(1-(2-methoxyquinoxalin-6-yl)ethyl)piperazin-1-yl)-4-methyl-5-oxo-4,5-dihydro-2H-pyrazolo[4,3-b]pyridin-2-yl)acetonitrile

### Step A: 6-bromo-2-methoxyquinoxaline

A solution of 6-bromo-2-chloroquinoxaline (2 g, 8.23 mmol) and NaOMe (5.4 M in MeOH, 6.1 ml, 32.94 mmol) in MeOH (15 ml) was stirred at 80° C. for 4 hours. The reaction mixture was concentrated to dryness. The resulting residue was dissolved in EA (30 ml). The resulting mixture was washed with brine (10 ml×2), dried over Na₂SO₄ and concentrated to give the titled compound (1.95 g, 100%). MS: M/e 239,241 (M+1)⁺.

### Step B: 1-(2-methoxyquinoxalin-6-yl)ethan-1-one

A solution of 6-bromo-2-methoxyquinoxaline (500 mg, 2.1 mmol), tributyl(1-ethoxyvinyl)stannane (986 mg, 2.73 mmol) and Pd(PPh₃)₂Cl₂ (147 mg, 0.21 mmol) in toluene (10 ml) was stirred at 100° C. overnight under N₂. The reaction mixture was cooled to RT and HCl (4 M in 1,4-dioxane, 5 ml) was added. The reaction mixture was stirred at RT for 30 minutes and concentrated under reduced pressure. The resulting residue was dissolved in EA (30 ml). The resulting mixture was washed with brine (15 ml), dried over Na₂SO₄ and concentrated to dryness. The resulting residue was purified by flash column chromatography (EA/PE) to give the titled compound (260 mg, 61%). MS: M/e 203 (M+1)⁺.

### Step C: 1-(2-methoxyquinoxalin-6-yl)ethan-1-ol

A solution of 1-(2-methoxyquinoxalin-6-yl)ethan-1-one (130 mg, 0.64 mmol) and NaBH₄ (25 mg, 0.66 mmol) in EtOH (3 ml) was stirred at RT for 10 minutes. After completed, the reaction mixture was concentrated to dryness. The resulting residue was dissolved in EA (30 ml). The resulting mixture was washed with brine (15 ml), dried over Na₂SO₄ and concentrated to give the titled compound (130 mg, 99%). MS: M/e 205 (M+1)⁺.

### Step D: 2-(7-((2S,5R)-2,5-diethyl-4-(1-(2-methoxyquinoxalin-6-yl)ethyl)piperazin-1-yl)-4-methyl-5-oxo-4,5-dihydro-2H-pyrazolo[4,3-b]pyridin-2-yl)acetonitrile

A solution of Intermediate 5 (140 mg, 0.43 mmol), 1-(2-methoxyquinoxalin-6-yl)ethan-1-ol (130 mg, 0.64 mmol), (cyanomethyl)trimethylphosphonium iodide (311 mg, 1.28 mmol) and DIEA (551 mg, 4.27 mmol) in MeCN (2 ml) was stirred at 100° C. overnight. The solvent was concentrated to dryness. The resulting residue was purified by flash column chromatography (MeOH/DCM) to give the titled Compound A281 (40 mg) as a mixture of diastereomers. The Compound A281 (10 mg) was separated by prep-HPLC (Method A) to give Compound A281a (1.2 mg) and Compound A281b (1.4 mg).

Compound A281a (the earlier peak): ¹H NMR (400 MHz, DMSO-d6) δ 8.59 (s, 1H), 7.97 (s, 1H), 7.94 (s, 1H), 7.84 (dd, J=20.2, 9.2 Hz, 2H), 5.60 (s, 2H), 5.40 (s, 1H), 4.04 (s, 3H), 3.96 (d, J=6.5 Hz, 1H), 3.31-3.28 (m, 2H), 3.27 (s, 3H), 3.13 (d, J=12.1 Hz, 1H), 2.97 (d, J=11.7 Hz, 1H), 2.82 (d, J=8.4 Hz, 1H), 2.32 (d, J=8.7 Hz, 1H), 2.13-1.98 (m, 1H), 1.72-1.61 (m, 1H), 1.59-1.42 (m, 2H), 1.37 (d, J=6.4 Hz, 3H), 0.87 (t, J=7.3 Hz, 3H), 0.59 (t, J=7.3 Hz, 3H) ppm. MS: M/e 515 (M+1)⁺.

Compound A281b (the later peak): ¹H NMR (400 MHz, DMSO-d6) δ 8.59 (s, 1H), 7.98 (s, 1H), 7.96 (s, 1H), 7.83 (s, 2H), 5.61 (s, 2H), 5.39 (s, 1H), 4.03 (s, 3H), 3.82 (d, J=6.5 Hz, 1H), 3.50-3.38 (m, 3H), 3.27 (s, 3H), 3.11 (d, J=10.5 Hz, 1H), 2.59 (d, J=10.9 Hz, 1H), 2.26 (d, J=12.0 Hz, 1H), 1.96-1.83 (m, 1H), 1.68-1.57 (m, 1H), 1.56-1.43 (m, 2H), 1.33 (d, J=6.3 Hz, 3H), 0.98 (t, J=7.1 Hz, 3H), 0.47 (t, J=7.1 Hz, 3H) ppm. MS: M/e 515 (M+1)⁺.

### Compound A282: 2-(7-((2S,5R)-2,5-diethyl-4-(1-(2-hydroxyquinoxalin-6-yl)ethyl)piperazin-1-yl)-4-methyl-5-oxo-4,5-dihydro-2H-pyrazolo[4,3-b]pyridin-2-yl)acetonitrile

A solution of 2-(7-((2S,5R)-2,5-diethyl-4-(1-(2-methoxyquinoxalin-6-yl)ethyl)piperazin-1-yl)-4-methyl-5-oxo-4,5-dihydro-2H-pyrazolo[4,3-b]pyridin-2-yl)acetonitrile (30 mg, 0.058 mmol) and HCl aq·(2 N, 2 ml) in dioxane (4 ml) was stirred at 100° C. for 2.5 hours. After completed, the solution was diluted with EA (15 ml), washed with brine (10 ml), dried over Na₂SO₄ and concentrated to dryness. The resulting residue was purified by prep-HPLC (Method A) to give two isomers of the titled compound, Compound A282a (3.5 mg) and Compound A282b (4.1 mg).

Compound A282a (the earlier peak): ¹H NMR (400 MHz, DMSO-d6) δ 12.41 (s, 1H), 8.16 (s, 1H), 7.97 (s, 1H), 7.72 (s, 1H), 7.59 (d, J=8.6 Hz, 1H), 7.31 (d, J=8.4 Hz, 1H), 5.62 (s, 2H), 5.39 (s, 1H), 3.83 (d, J=6.7 Hz, 1H), 3.27 (s, 5H), 3.10 (d, J=12.7 Hz, 1H), 2.93 (d, J=11.7 Hz, 1H), 2.79 (d, J=8.4 Hz, 1H), 2.32 (s, 1H), 2.10-1.95 (m, 1H), 1.70-1.58 (m, 1H), 1.55-1.41 (m, 2H), 1.31 (d, J=6.4 Hz, 3H), 0.88 (t, J=7.4 Hz, 3H), 0.61 (t, J=7.3 Hz, 3H) ppm. MS: M/e 501 (M+1)⁺.

Compound A282b (the later peak): ¹H NMR (400 MHz, DMSO-d) δ 12.41 (s, 1H), 8.16 (s, 1H), 7.98 (s, 1H), 7.75 (s, 1H), 7.60 (d, J=8.3 Hz, 1H), 7.28 (d, J=8.4 Hz, 1H), 5.61 (s, 2H), 5.39 (s, 1H), 3.69 (q, J=6.5 Hz, 1H), 3.39 (s, 3H), 3.27 (s, 3H), 3.06 (d, J=9.4 Hz, 1H), 2.55 (d, J=9.1 Hz, 1H), 2.26 (d, J=12.1 Hz, 1H), 1.94-1.79 (m, 1H), 1.61-1.42 (m, 3H), 1.27 (d, J=6.4 Hz, 3H), 0.95 (t, J=7.1 Hz, 3H), 0.51 (t, J=7.1 Hz, 3H) ppm. MS: M/e 501 (M+1)⁺.

### Compound A284: 2-(7-((2S,5R)-4-(1-(2-hydroxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

A solution of 2-(7-((2S,5R)-4-(1-(2-methoxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile (80 mg, 0.16 mmol) and HCl aq·(2 N, 2 ml) in 1,4-dioxane (4 ml) was stirred at 100° C. for 1.5 hours. After completed, the reaction solution was diluted with EA (15 ml), washed with brine (10 ml), dried over Na₂SO₄ and concentrated to dryness. The resulting residue was purified by prep-TLC to give the titled compound (40 mg). ¹H NMR (400 MHz, DMSO-d6) δ 12.41 (s, 1H), 8.16 (s, 1H), 7.99 (s, 1H), 7.75 (d, J=7.0 Hz, 1H), 7.61 (t, J=9.7 Hz, 1H), 7.30 (dd, J=13.6, 8.5 Hz, 1H), 5.61 (s, 2H), 5.41 (d, J=7.0 Hz, 1H), 5.01-4.27 (m, 1H), 3.72 (q, J=6.6 Hz, 0.5H), 3.59 (q, J=6.9 Hz, 0.5H), 3.56-3.46 (m, 1H), 3.43 (s, 0.5H), 3.27 (s, 3H), 3.23 (s, 0.5H), 3.04-2.87 (m, 1H), 2.80 (d, J=11.6 Hz, 1H), 2.66 (d, J=10.9 Hz, 0.5H), 2.11 (d, J=12.4 Hz, 0.5H), 1.29 (dd, J=13.2, 6.9 Hz, 4.5H), 1.08 (dd, J=6.1, 3.3 Hz, 3H), 0.93 (d, J=6.5 Hz, 1.5H) ppm. MS: M/e 473 (M+1)⁺.

### Compound A286: 2-(7-((2S,5R)-4-(1-(4-fluoro-2-(1-methoxyethyl)phenyl)ethyl)-2,5-dimethylpiperazin-1-yl)-4-methyl-5-oxo-4,5-dihydro-2H-pyrazolo[4,3-b]pyridin-2-yl)acetonitrile

### Step A: 1-(2-bromo-5-fluorophenyl)ethan-1-ol

To a solution of 1-(2-bromo-5-fluorophenyl)ethan-1-one (21.7 g, 100 mmol) in MeOH (150 mL) was added NaBH₄ (2.28 g, 60 mmol) at 0° C. in portions and the reaction mixture was stirred at room temperature for 16 hours. The reaction mixture was concentrated to dryness. The resulting residue was treated with saturated NaHCO₃ aq·(50 mL). The resulting aqueous was extracted with EA (50 mL×2). The combined organic layers were washed with brine, dried over Na₂SO₄ and concentrated to give the titled compound (21.5 g, 98%) as a racemate. ¹H NMR (400 MHz, DMSO-d6) δ 7.59 (dd, J=8.8, 5.6 Hz, 1H), 7.35 (dd, J=10.0, 3.2 Hz, 1H), 7.07 (td, J=8.4, 3.2 Hz, 1H), 5.55 (d, J=4.0 Hz, 1H), 4.97-4.80 (m, 1H), 1.29 (d, J=6.4 Hz, 3H).

### Step B: 1-bromo-4-fluoro-2-(1-methoxyethyl)benzene

To a solution of 1-(2-bromo-5-fluorophenyl)ethan-1-ol (10.0 g, 45.6 mmol) in THF (100 mL) was added NaH (5.0 g, 125 mmol) at 0° C. in portions and the reaction mixture was stirred at room temperature for 10 minutes. Mel (10.0 g, 70.4 mmol) was added and the mixture was stirred at room temperature for 16 hours. The reaction mixture was poured into 100 mL of H₂O and extracted with EA (50 mL×2). The combined organic layers was washed with brine, dried over Na₂SO₄ and concentrated to give the titled compound (10 g, 94%) as a racemate. ¹H NMR (400 MHz, DMSO-d6) δ 7.65 (dd, J=8.8, 5.2 Hz, 1H), 7.21 (dd, J=10.0, 3.2 Hz, 1H), 7.14 (td, J=8.4, 3.2 Hz, 1H), 4.64-4.52 (m, 1H), 3.18 (s, 3H), 1.32 (d, J=6.4 Hz, 4H).

### Step C: 1-(4-fluoro-2-(1-methoxyethyl)phenyl)ethan-1-one

A mixture of 1-bromo-4-fluoro-2-(1-methoxyethyl)benzene (5.0 g, 21.4 mmol), tributyl(1-ethoxyvinyl)stannane (10.1 g, 28.0 mmol) and Pd(PPh₃)₂Cl₂ (750 mg, 1.07 mmol) in toluene (100 mL) was stirred at 100° C. under N₂ for 16 hours. The reaction mixture was cooled to RT and HCl (4M in 1,4-dioxane, 15 mL) was added. The resulting mixture was stirred for 10 minutes. The resulting mixture was diluted with EA (100 mL), washed with brine (100 mL×2), dried and concentrated to dryness. The resulting residue was purified by flash column chromatography to give the titled compound (1.6 g, 37%) as a racemate. ¹H NMR (400 MHz, DMSO-d6) δ 7.96 (dd, J=8.4, 5.6 Hz, 1H), 7.31 (dd, J=10.4, 2.8 Hz, 1H), 7.25 (td, J=8.8, 2.8 Hz, 1H), 4.93-4.74 (m, 1H), 3.10 (s, 3H), 2.58 (s, 3H), 1.32 (d, J=6.4 Hz, 3H).

### Step D: 1-(4-fluoro-2-(1-methoxyethyl)phenyl)ethan-1-ol

To a solution of 1-(4-fluoro-2-(1-methoxyethyl)phenyl)ethan-1-one (1.6 g, 8.2 mmol) in MeOH (20 mL) was added NaBH₄ (186 mg, 4.9 mmol) at 0° C. and the mixture was stirred at room temperature for 16 hours. The reaction mixture was concentrated to dryness. The resulting residue was dissolved into EA (50 mL). The organic layer was washed with brine (20 mL×3), dried over Na₂SO₄ and concentrated to dryness. The resulting residue was purified by flash column chromatography to give the titled compound (760 mg, 48%) as a mixture. ¹H NMR (400 MHz, DMSO-d6) δ 7.56-7.45 (m, 1H), 7.15-6.99 (m, 2H), 5.25-5.06 (m, 1H), 5.06-4.86 (m, 1H), 4.78-4.60 (m, 1H), 3.18-3.05 (m, 3H), 1.41-1.25 (m, 6H).

### Step E: 7-((2S,5R)-4-(1-(4-fluoro-2-(1-methoxyethyl)phenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

A mixture of Intermediate 10B (100 mg, 0.29 mmol), 1-(4-fluoro-2-(1-methoxyethyl)phenyl)ethan-1-ol (150 mg, 0.76 mmol), DIEA (225 mg, 1.74 mmol) and (cyanomethyl)trimethylphosphonium iodide (247 mg, 1.01 mmol) in MeCN (1 mL) was stirred at 100° C. for 24 hours. The resulting mixture was diluted with EA (10 mL), washed with brine (5 mL×3), dried and concentrated to dryness. The resulting residue was purified by flash column chromatography to give the titled compound (85 mg, 56%) as a mixture. MS: M/e 526 (M+1)⁺.

### Step F: 7-((2S,5R)-4-(1-(4-fluoro-2-(1-methoxyethyl)phenyl)ethyl)-2,5-dimethylpiperazin-1-yl)-4-methyl-2,4-dihydro-5H-pyrazolo[4,3-b]pyridin-5-one

To a solution of 7-((2S,5R)-4-(1-(4-fluoro-2-(1-methoxyethyl)phenyl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (80 mg, 0.15 mmol) in MeOH (4 mL) was added HCl (2 mL, 4M in 1,4-dioxane) at room temperature. The resulting mixture was stirred at room temperature for 3 hours. The resulting mixture was concentrated to give the crude titled compound (75 mg), which was used to the next step directly without further purification. MS: M/e 442 (M+1)⁺.

### Step G: 2-(7-((2S,5R)-4-(1-(4-fluoro-2-(1-methoxyethyl)phenyl)ethyl)-2,5-dimethylpiperazin-1-yl)-4-methyl-5-oxo-4,5-dihydro-2H-pyrazolo[4,3-b]pyridin-2-yl)acetonitrile

To a mixture of 7-((2S,5R)-4-(1-(4-fluoro-2-(1-methoxyethyl)phenyl)ethyl)-2,5-dimethylpiperazin-1-yl)-4-methyl-2,4-dihydro-5H-pyrazolo[4,3-b]pyridin-5-one (75 mg, crude), K₂CO₃ (104 mg, 0.75 mmol) and H₂O (27 mg, 1.5 mmol) in DMF (1.5 mL) was added 2-iodoacetonitrile (34 mg, 0.45 mmol). The resulting mixture was stirred at room temperature for 16 hours. The reaction mixture was diluted with EA (20 mL). The organic layer was washed with brine (5 mL×3), dried over Na₂SO₄, and concentrated to dryness. The resulting residue was purified by flash column chromatography to obtain Compound A286, and furtherly purified by prep-HPLC (Method A) to give Compound A286a (6.5 mg) and Compound A286b (8.0 mg) as a mixture of two isomers respectively.

Compound A286a (the earlier peak): ¹H NMR (400 MHz, CD₃OD) δ 7.92 (s, 1H), 7.79-7.66 (m, 1H), 7.14-7.04 (m, 1H), 7.04-6.94 (m, 1H), 5.60-5.53 (m, 1H), 5.47 (s, 2H), 4.83-4.71 (m, 1H), 4.68-4.39 (m, 2H), 3.95-3.82 (m, 1H), 3.70-3.55 (m, 2H), 3.43 (s, 3H), 3.25-3.18 (m, 3H), 2.87-2.79 (m, 1H), 2.26-2.13 (m, 1H), 1.41-1.26 (m, 6H), 1.24-1.14 (m, 6H). MS: M/e 481 (M+1)⁺.

Compound A286b (the later peak): ¹H NMR (400 MHz, CD₃OD) δ 7.92 (s, 1H), 7.71-7.51 (m, 1H), 7.21-7.07 (m, 1H), 7.05-6.93 (m, 1H), 5.56 (s, 1H), 5.47 (s, 2H), 5.03-4.86 (m, 2H), 3.98-3.87 (m, 1H), 3.45-3.37 (m, 4H), 3.27-3.20 (m, 3H), 3.07-2.50 (m, 4H), 1.47-1.34 (m, 6H), 1.33-1.25 (m, 3H), 1.07-0.95 (m, 3H). MS: M/e 481 (M+1)⁺.

### Compound A287: 2-(7-((2S,5R)-4-(1-(2-(cyanomethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile

Compound A287, and its separated isomers Compound A287a (10 mg) and Compound A287b (9 mg) were prepared according to the procedures described for Compound A29, Compound A29a and Compound A29b under appropriate conditions that could be recognized by one skilled in the art.

Compound A287a (the earlier peak): ¹H NMR (400 MHz, CD₃OD) δ 7.92 (s, 1H), 7.71-7.57 (m, 1H), 7.15 (dd, J=9.6, 2.8 Hz, 1H), 7.09 (td, J=8.4, 2.8 Hz, 1H), 5.56 (s, 1H), 5.47 (s, 2H), 4.75-4.30 (m, 2H), 4.25-4.06 (m, 2H), 3.86-3.75 (m, 1H), 3.68-3.55 (m, 2H), 3.43 (s, 3H), 2.84 (dd, J=12.0, 4.0 Hz, 1H), 2.13 (d, J=11.2 Hz, 1H), 1.37 (d, J=6.4 Hz, 3H), 1.23-1.13 (m, 6H). MS: M/e 462 (M+1)⁺.

Compound A287b (the later peak): ¹H NMR (400 MHz, CD₃OD) δ 7.92 (s, 1H), 7.51 (dd, J=8.4, 6.0 Hz, 1H), 7.20 (dd, J=9.6, 2.4 Hz, 1H), 7.08 (td, J=8.4, 2.4 Hz, 1H), 5.57 (s, 1H), 5.47 (s, 2H), 5.09-4.86 (m, 2H), 4.36 (d, J=18.4 Hz, 1H), 4.25 (d, J=18.0 Hz, 1H), 3.92-3.84 (m, 1H), 3.50 (dd, J=13.2, 3.2 Hz, 1H), 3.43 (s, 3H), 2.96 (dd, J=12.0, 4.0 Hz, 1H), 2.92-2.83 (m, 2H), 1.40-1.33 (m, 6H), 1.00 (d, J=6.8 Hz, 3H). MS: M/e 462 (M+1)⁺.

### Compound A294: 2-(7-((2S,5R)-4-(1-(2,3-dimethylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 6-bromo-2,3-dimethylquinoxaline

To a solution of 4-bromobenzene-1,2-diamine (2.5 g, 13.37 mmol) in MeOH (40 mL) was added biacetyl (1.26 g, 14.70 mmol). The mixture solution was stirred at room temperature for 2 hours. Then the reaction solution was concentrated under reduced pressure to give the titled compound (3 g, 95%). MS: M/e 236 (M+1)⁺.

### Step B: 1-(2,3-dimethylquinoxalin-6-yl)ethan-1-one

To a solution of 6-bromo-2,3-dimethylquinoxaline (1.28 g, 5.424 mmol), Pd(PPh₃)₂Cl₂ (762 mg, 1.085 mmol) and tributyl(1-ethoxyvinyl)stannane (3.92 g, 10.847 mmol) in toluene (30 mL) was degassed 3 times under N₂ atmosphere. The mixture solution was stirred at 90° C. for 12 hours. Then added HCl (4 mL, 4M in 1,4-dioxane), the mixture solution was stirred at RT for 4 hours. The reaction solution was concentrated under reduced pressure to dryness. The resulting residue was purified by flash column chromatography to give the titled compound (800 mg, 74%). MS: M/e 201 (M+1)⁺.

### Step C: 1-(2,3-dimethylquinoxalin-6-yl)ethan-1-ol

To a solution of 1-(2,3-dimethylquinoxalin-6-yl)ethan-1-one (800 mg, 4.0 mmol) in MeOH (30 mL) was added NaBH₄ (152 mg, 4.0 mmol). The mixture was stirred at room temperature for 1 hours. The reaction mixture was quenched with saturated NH₄Cl (20 mL) at RT and extracted with EA (50 mL×2). The combined organic layers were dried over Na₂SO₄ and concentrated under reduced pressure. The residue was purified by column chromatography to give the titled compound (727 mg, 90%). MS: M/e 203 (M+1)⁺.

### Step D: 7-((2S,5R)-4-(1-(2,3-dimethylquinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

To a solution of 1-(2,3-dimethylquinoxalin-6-yl)ethan-1-ol (176 mg, 0.870 mmol), Intermediate 10B (150 mg, 0.435 mmol) and (cyanomethyl)trimethylphosphonium iodide (211 mg, 0.870 mmol) in CH₃CN (2 mL) was added DIEA (168 mg, 1.305 mmol). The mixture solution was degassed 3 times under N₂ atmosphere. Then the mixture solution was stirred at 105° C. for 24 hours. The reaction mixture was quenched with saturated NH₄Cl (20 mL) at room temperature and extracted with EA (30 mL×2). The combined organic layers were dried over Na₂SO₄ and concentrated under reduced pressure. The residue was purified by flash column chromatography to give the titled compound (220 mg, 96%). MS: M/e 530 (M+1)⁺.

### Step E: 7-((2S,5R)-4-(1-(2,3-dimethylquinoxalin-6-yl)ethyl)-2,5-dimethylpiperazin-1-yl)-4-methyl-2,4-dihydro-5H-pyrazolo[4,3-b]pyridin-5-one

To a solution of 7-((2S,5R)-4-(1-(2,3-dimethylquinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (220 mg, 0.416 mmol) in DCM (10 mL) at room temperature was added HCl (5 mL, 4M in 1,4-dioxane). The mixture was stirred at room temperature for 2 hours. Then the mixture was concentrated under reduced pressure to give the crude product (HCl salt). The crude product was basified by Na₂CO₃ (4M) to pH-10 and extracted with EA (35 mL×3). The combined organic layers were washed with brine (20 mL×3), dried over Na₂SO₄ and concentrated under reduced pressure to give the titled compound (170 mg, 92%). MS: M/e 446 (M+1)⁺.

### Step F: 2-(7-((2S,5R)-4-(1-(2,3-dimethylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 7-((2S,5R)-4-(1-(2,3-dimethylquinoxalin-6-yl)ethyl)-2,5-dimethylpiperazin-1-yl)-4-methyl-2,4-dihydro-5H-pyrazolo[4,3-b]pyridin-5-one (170 mg, 0.382 mmol) and K₂CO₃ (105 mg, 0.764 mmol) in DMF (8 mL) at room temperature was added 2-chloroacetonitrile (43 mg, 0.573 mmol). The mixture solution was stirred at room temperature for 12 hours. Then the reaction mixture was quenched with saturated NaCl (20 mL) at room temperature and extracted with EA (30 mL×2). The combined organic layers were dried over Na₂SO₄ and concentrated under reduced pressure. The resulting residue was purified by prep-HPLC (Method A) to give the titled compound. (70 mg, 38%). ¹H NMR (400 MHz, CD₃OD) δ 7.98-7.90 (m, 3H), 7.89-7.85 (m, 1H), 5.56 (s, 1H), 5.46 (d, J=4.0 Hz, 2H), 4.68-4.62 (m, 1H), 4.34-4.20 (m, 0.5H), 3.96-3.88 (m, 0.5H), 3.83-3.75 (m, 0.5H), 3.70-3.67 (m, 1H), 3.48-3.44 (m, 0.5H), 3.43 (s, 3H), 3.12-3.06 (m, 0.5H), 2.94-2.91 (m, 1H), 2.90-2.82 (m, 0.5H), 2.78-2.73 (m, 0.5H), 2.74 (s, 6H), 2.26-2.18 (m, 0.5H), 1.46-1.41 (m, 4.5H), 1.24-1.19 (m, 3.5H), 1.08-1.02 (m, 1H). MS: M/e 485 (M+1)⁺.

### Compound A295: 2-(7-((3R,4R)-3-ethoxy-4-(3-(trifluoromethyl)phenoxy)piperidin-1-yl)-4-methyl-5-oxo-4,5-dihydro-2H-pyrazolo[4,3-b]pyridin-2-yl)acetonitrile

### Step A: tert-butyl (3R,4R)-3-hydroxy-4-(3-(trifluoromethyl)phenoxy)piperidine-1-carboxylate

To a solution of tert-butyl 7-oxa-3-azabicyclo[4.1.0]heptane-3-carboxylate (1.5 g, 7.53 mmol) and K₂CO₃ (1.04 g, 7.53 mmol) in EtOH (20 mL) was added 3-(trifluoromethyl)phenol (1.22 g, 7.53 mmol) at room temperature. The reaction mixture was stirred at 80° C. for 3 hours. The reaction mixture was filtered, washed with excess EtOH. The filtrates were concentrated to dryness. The resulting residue was purified by flash column chromatography to give the titled compound (1.3 g, mixture, contained tert-butyl (3R,4R)-4-hydroxy-3-(3-(trifluoromethyl)phenoxy)piperidine-1-carboxylate). MS: M/e 362 (M+1)⁺.

### Step B: tert-butyl (3R,4R)-3-ethoxy-4-(3-(trifluoromethyl)phenoxy)piperidine-1-carboxylate

To a solution of tert-butyl (3R,4R)-3-hydroxy-4-(3-(trifluoromethyl)phenoxy)piperidine-1-carboxylate (1.3 g, mixture, 3.6 mmol, contained tert-butyl (3R,4R)-4-hydroxy-3-(3-(trifluoromethyl)phenoxy)piperidine-1-carboxylate) in THF (20 mL) was added NaH (0.43 g, 10.8 mmol, 60% in mineral oil) slowly at 0° C. The reaction mixture was stirred at 0° C. for 30 mins. A solution of Iodoethane (1.1 g, 7.2 mmol) in THF (10 mL) was added. The reaction was stirred at reflux overnight. The reaction was cooled at 0° C., quenched by H₂O and extracted with EtOAc. The organic layers were concentrated and purified by flash column chromatography to give the titled compound (0.7 g, 73%, mixture, contained tert-butyl (3R,4R)-4-ethoxy-3-(3-(trifluoromethyl)phenoxy)piperidine-1-carboxylate). MS: M/e 390 (M+1)⁺.

### Step C: (3R,4R)-3-ethoxy-4-(3-(trifluoromethyl)phenoxy)piperidine

To a solution of tert-butyl (3R,4R)-3-ethoxy-4-(3-(trifluoromethyl)phenoxy)piperidine-1-carboxylate (0.7 g, 1.8 mmol, contained tert-butyl (3R,4R)-4-ethoxy-3-(3-(trifluoromethyl)phenoxy)piperidine-1-carboxylate) in DCM (10 mL) was added HCl (10 mL, 10 mmol, 4M in Dioxane) slowly at 0° C. The resulting mixture was stirred at room temperature for 2 hours. The solvent was removed under reduced pressure. The resulting residue was neutralized by aq·NaHCO₃ and extracted with DCM. The organic layers were concentrated and purified by flash column chromatography to give the titled compound (0.45 g, 57%, mixture, contained (3R,4R)-4-ethoxy-3-(3-(trifluoromethyl)phenoxy)piperidine). MS: M/e 290 (M+1)⁺.

### Step D: 7-((3R,4R)-3-ethoxy-4-(3-(trifluoromethyl)phenoxy)piperidin-1-yl)-4-methyl-2-(tetrahydro-2H-pyran-2-yl)-2,4-dihydro-5H-pyrazolo[4,3-b]pyridin-5-one

A mixture of 4-methyl-5-oxo-2-(tetrahydro-2H-pyran-2-yl)-4,5-dihydro-2H-pyrazolo[4,3-b]pyridin-7-yl trifluoromethanesulfonate (193 mg, 0.51 mmol), (3R,4R)-3-ethoxy-4-(3-(trifluoromethyl)phenoxy)piperidine (150 mg, 0.51 mmol, contained (3R,4R)-4-ethoxy-3-(3-(trifluoromethyl)phenoxy)piperidine) and DIPEA (193 mg, 1.5 mmol) in MeCN (2 ml) was stirred at 90° C. overnight. The solvent was removed under reduced pressure. The resulting residue was purified by flash column chromatography to give the titled compound (160 mg, 59%, mixture, 7-((3R,4R)-4-ethoxy-3-(3-(trifluoromethyl)phenoxy)piperidin-1-yl)-4-methyl-2-(tetrahydro-2H-pyran-2-yl)-2,4-dihydro-5H-pyrazolo[4,3-b]pyridin-5-one). MS: M/e 521 (M+1)⁺.

### Step E: 7-((3R,4R)-3-ethoxy-4-(3-(trifluoromethyl)phenoxy)piperidin-1-yl)-4-methyl-2,4-dihydro-5H-pyrazolo[4,3-b]pyridin-5-one

To a solution of 7-((3R,4R)-3-ethoxy-4-(3-(trifluoromethyl)phenoxy)piperidin-1-yl)-4-methyl-2-(tetrahydro-2H-pyran-2-yl)-2,4-dihydro-5H-pyrazolo[4,3-b]pyridin-5-one (40 mg, 0.08 mmol, contained 7-((3R,4R)-4-ethoxy-3-(3-(trifluoromethyl)phenoxy)piperidin-1-yl)-4-methyl-2-(tetrahydro-2H-pyran-2-yl)-2,4-dihydro-5H-pyrazolo[4,3-b]pyridin-5-one) in DCM (2 mL) was added TFA (2 mL). The resulting mixture was stirred at room temperature overnight. The reaction mixture was concentrated under reduced pressure, diluted with the mixture of water/DCM, basified with saturated NaHCO₃ solution to pH 7-8 and extracted with DCM (60 mL×2). The combined organic layers were washed with brine, dried over Na₂SO₄, filtered and concentrated. The resulting residue was purified by prep-TLC to give the titled compound (40 mg, 42%, mixture, contained 7-((3R,4R)-4-ethoxy-3-(3-(trifluoromethyl)phenoxy)piperidin-1-yl)-4-methyl-2,4-dihydro-5H-pyrazolo[4,3-b]pyridin-5-one). MS: M/e 437 (M+1)⁺.

### Step F: 2-(7-((3R,4R)-3-ethoxy-4-(3-(trifluoromethyl)phenoxy)piperidin-1-yl)-4-methyl-5-oxo-4,5-dihydro-2H-pyrazolo[4,3-b]pyridin-2-yl)acetonitrile

To a solution of 7-((3R,4R)-3-ethoxy-4-(3-(trifluoromethyl)phenoxy)piperidin-1-yl)-4-methyl-2,4-dihydro-5H-pyrazolo[4,3-b]pyridin-5-one (40 mg, 0.09 mmol, mixture, contained 7-((3R,4R)-4-ethoxy-3-(3-(trifluoromethyl)phenoxy)piperidin-1-yl)-4-methyl-2,4-dihydro-5H-pyrazolo[4,3-b]pyridin-5-one) and K₂CO₃ (25 mg, 0.18 mmol) in DMF (2 mL) was added 2-iodoacetonitrile (25 mg, 0.15 mmol). The reaction mixture was stirred at room temperature overnight. The reaction mixture was diluted with water and extracted with EA (10 mL×2). The combined organic layers were washed with brine, dried over Na₂SO₄, filtered and concentrated to dryness. The resulting residue was purified by prep-TLC to give the titled compound (2 mg, mixture, contained 15% 2-(7-((3R,4R)-4-ethoxy-3-(3-(trifluoromethyl)phenoxy)piperidin-1-yl)-4-methyl-5-oxo-4,5-dihydro-2H-pyrazolo[4,3-b]pyridin-2-yl)acetonitrile). ¹H NMR (400 MHz, CD₃OD) δ 7.95 (s, 1H), 7.47-7.45 (m, 1H), 7.30-7.23 (m, 3H), 5.68 (s, 1H), 5.50 (s, 2H), 4.58-4.54 (m, 3H), 4.12-4.05 (m, 1H), 3.95-3.92 (m, 1H), 3.76-3.74 (m, 1H), 3.57-3.53 (m, 2H), 3.45 (s, 3H), 2.30-2.25 (m, 1H), 1.82-1.80 (m, 1H), 1.06-0.88 (m, 3H) ppm. MS: M/e 476 (M+1)⁺.

### Compound A296: 2-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)-N-(2-methoxyethyl)-2-(4-(trifluoromethyl)phenyl)acetamide

### Step A: ethyl 2-bromo-2-(4-(trifluoromethyl)phenyl)acetate

A solution of ethyl 2-(4-(trifluoromethyl)phenyl)acetate (2 g, 8.6 mmol), NBS (1.8 g, 10.3 mmol) in DCM (30 mL) was stirred at r.t overnight. The mixture was quenched with water (10 mL), extracted with dichloromethane (100 mL×3) and washed with brine (30 mL×2), dried over Na₂SO₄, and concentrated to give residue. The resulting residue was purified by flash column chromatography (MeOH:DCM=0-10%) to give the titled compound (2.4 g, 90%). MS: M/e 311 (M+1)⁺.

### Step B: ethyl 2-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)-2-(4-(trifluoromethyl)phenyl)acetate

A mixture of Intermediate 3 (200 mg, 0.54 mmol), ethyl 2-bromo-2-(4-(trifluoromethyl)phenyl)acetate (166 mg, 0.54 mmol) and DIPEA (138 mg, 1.1 mmol) in CH₃CN (2 mL) was heated to 80° C. overnight under N₂ atmosphere. The solvent was removed under vacuum. The resulting residue was purified by Pre-TLC (DCM:MeOH=15:1) to give the titled compound (150 mg, 46%). MS: M/e 604 (M+1)⁺.

### Step C: 2-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)-2-(4-(trifluoromethyl)phenyl)acetic acid

A mixture of ethyl 2-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)-2-(4-(trifluoromethyl)phenyl)acetate (150 mg, 0.25 mmol) and NaOH (20 mg, 0.5 mmol) in MeOH (10 mL) was stirred at RT overnight. The mixture was quenched by HCl aq. and the solvent was removed under vacuum. The resulting residue was purified by Pre-TLC (DCM:MeOH=15:1) to give the titled compound (130 mg, 91%). MS: M/e 576 (M+1)⁺.

### Step D: 2-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)-N-(2-methoxyethyl)-2-(4-(trifluoromethyl)phenyl)acetamide

To a solution of 2-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)-2-(4-(trifluoromethyl)phenyl)acetic acid (130 mg, 0.22 mol) and 2-methoxyethan-1-amine (17 mg, 0.22 mol) in DCM (10 mL) were added HATU (172 mg, 0.45 mmol) and TEA (114 mg, 1.13 mol). The mixture was stirred at RT for 4 hours. After completed, the solution was concentrated under reduced pressure. The resulting residue was purified by Prep-TLC to give the titled compound (130 mg, 91%). MS: M/e 633 (M+1)⁺.

### Step E: 2-((2R,5S)-2,5-diethyl-4-(4-methyl-5-oxo-4,5-dihydro-2H-pyrazolo[4,3-b]pyridin-7-yl)piperazin-1-yl)-N-(2-methoxyethyl)-2-(4-(trifluoromethyl)phenyl)acetamide

To a solution of 2-((2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazin-1-yl)-N-(2-methoxyethyl)-2-(4-(trifluoromethyl)phenyl)acetamide (130 mg, 0.21 mmol) in MeOH (2 mL) was added HCl dioxane solution (0.5 mL, 2 mmol, 4M) at room temperature. The resulting mixture was stirred at room temperature for another 2 hours. The reaction solvent was removed under vacuum. The crude product was dissolved with DCM (10 mL) and neutralized with aq·NaOH to pH=8. The organic layer was concentrated to dryness. The resulting residue was further purified by Prep-TLC (DCM:MeOH=10:1) to give the titled compound (80 mg, 71%). MS: M/e 549 (M+1)⁺.

### Step F: 2-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)-N-(2-methoxyethyl)-2-(4-(trifluoromethyl)phenyl)acetamide

To a solution of 2-((2R,5S)-2,5-diethyl-4-(4-methyl-5-oxo-4,5-dihydro-2H-pyrazolo[4,3-b]pyridin-7-yl)piperazin-1-yl)-N-(2-methoxyethyl)-2-(4-(trifluoromethyl)phenyl)acetamide (100 mg, 0.18 mmol) and K₂CO₃ (50 mg, 0.37 mmol) in DMF (3 mL) was added 2-iodoacetonitrile (46 mg, 0.27 mmol). The reaction mixture was stirred at room temperature overnight. The reaction mixture was diluted with water and extracted with EA (80 mL×2). The combined organic layers were washed with brine, dried over Na₂SO₄, filtered and concentrated to dryness. The resulting residue was purified by flash column chromatography to give the titled compound (64 mg, 60%). ¹H NMR (400 MHz, CD₃OD) δ 7.92 (s, 1H), 7.74 (t, J=8.2 Hz, 2H), 7.71-7.62 (m, 2H), 5.57 (d, J=8.9 Hz, 1H), 5.46 (d, J=2.3 Hz, 2H), 4.36-4.24 (m, 1H), 3.59 (d, J=13.9 Hz, 1H), 3.46-3.42 (m, 5H), 3.41-3.34 (m, 2H), 3.33 (s, 3H), 3.05-2.92 (m, 1H), 2.88-2.46 (m, 2H), 2.45-1.98 (m, 2H), 1.98-1.72 (m, 1H), 1.71-1.64 (m, 1H), 1.62-1.23 (m, 2H), 1.04-0.91 (m, 3H), 0.79-0.51 (m, 3H). MS: M/e 588 (M+1)⁺

### Compound A297: 2-(7-((2S,5R)-4-(2-(4-fluorophenyl)propan-2-yl)-2,5-dimethylpiperazin-1-yl)-4-methyl-5-oxo-4,5-dihydro-2H-pyrazolo[4,3-b]pyridin-2-yl)acetonitrile

### Step A: 1-(2-chloropropan-2-yl)-4-fluorobenzene

A solution of 2-(4-fluorophenyl)propan-2-ol (2 g, 12.99 mmol) and SOCl₂ (2.32 g, 19.50 mmol) in DCM (25 ml) was stirred at RT overnight. The reaction mixture was concentrated to dryness to give the titled compound (2.2 g, 100%). ¹H NMR (400 MHz, CDCl₃) δ 7.58-7.52 (m, 2H), 7.06-6.98 (m, 2H), 1.98 (s, 6H) ppm

### Step B: tert-butyl (2S,5R)-4-(2-(4-fluorophenyl)propan-2-yl)-2,5-dimethylpiperazine-1-carboxylate

A solution of 1-(2-chloropropan-2-yl)-4-fluorobenzene (2.2 g, 12.72 mmol) and tert-butyl (2S,5R)-2,5-dimethylpiperazine-1-carboxylate (5.44 g, 25.42 mmol) in MeCN (12 ml) was stirred at 80° C. overnight. The reaction mixture was concentrated. The resulting residue was purified by flash column chromatography with 0-20% EA in PE to give the titled compound (320 mg, 7.2%). ¹H NMR (400 MHz, CDCl₃) δ 7.52-7.45 (m, 2H), 6.96 (t, J=8.7 Hz, 2H), 4.22-3.90 (m, 1H), 3.61 (s, 1H), 3.27 (s, 2H), 2.84 (dd, J=12.1, 4.1 Hz, 1H), 2.11 (dd, J=12.0, 2.3 Hz, 1H), 1.44 (s, 9H), 1.38 (d, J=7.6 Hz, 6H), 1.06 (t, J=6.8 Hz, 6H) ppm.

### Step C: (2R,5S)-1-(2-(4-fluorophenyl)propan-2-yl)-2,5-dimethylpiperazine

A solution of tert-butyl (2S,5R)-4-(2-(4-fluorophenyl)propan-2-yl)-2,5-dimethylpiperazine-1-carboxylate (160 mg, 0.48 mmol) and HCl (4M in dioxane, 4 ml, 16 mmol) in MeOH (5 ml) was stirred at RT for 2.5 h. The reaction mixture was concentrated to dryness to give the titled compound (114 mg, 100%). MS: M/e 251 (M+1)⁺

### Step D: 7-((2S,5R)-4-(2-(4-fluorophenyl)propan-2-yl)-2,5-dimethylpiperazin-1-yl)-4-methyl-2-(tetrahydro-2H-pyran-2-yl)-2,4-dihydro-5H-pyrazolo[4,3-b]pyridin-5-one

A solution of (2R,5S)-1-(2-(4-fluorophenyl)propan-2-yl)-2,5-dimethylpiperazine (114 mg, 0.46 mmol), Intermediate 2 (261 mg, 0.69 mmol) and DIPEA (176 mg, 1.36 mmol) in CH₃CN (4 ml) was stirred at 100° C. overnight. The mixture was concentrated to dryness. The resulting residue was purified by flash column chromatography with 0-5% MeOH in DCM to give the titled compound (200 mg, 91%). MS: M/e 482 (M+1)⁺

### Step E: 7-((2S,5R)-4-(2-(4-fluorophenyl)propan-2-yl)-2,5-dimethylpiperazin-1-yl)-4-methyl-2,4-dihydro-5H-pyrazolo[4,3-b]pyridin-5-one

A solution of 7-((2S,5R)-4-(2-(4-fluorophenyl)propan-2-yl)-2,5-dimethylpiperazin-1-yl)-4-methyl-2-(tetrahydro-2H-pyran-2-yl)-2,4-dihydro-5H-pyrazolo[4,3-b]pyridin-5-one (100 mg, 0.21 mmol) and HCl (4M in dioxane, 1.5 ml, 6 mmol) in MeOH (1.5 ml) was stirred at RT overnight. The mixture was concentrated to dryness to give the titled compound (82.5 mg, 100%). MS: M/e 398 (M+1)⁺

### Step F: 2-(7-((2S,5R)-4-(2-(4-fluorophenyl)propan-2-yl)-2,5-dimethylpiperazin-1-yl)-4-methyl-5-oxo-4,5-dihydro-2H-pyrazolo[4,3-b]pyridin-2-yl)acetonitrile

A solution of 7-((2S,5R)-4-(2-(4-fluorophenyl)propan-2-yl)-2,5-dimethylpiperazin-1-yl)-4-methyl-2,4-dihydro-5H-pyrazolo[4,3-b]pyridin-5-one (82.5 mg, 0.21 mmol), 2-chloroacetonitrile (63.2 mg, 0.83 mmol) and K₂CO₃ (86 mg, 0.62 mmol) in DMF (2 ml) was stirred at RT for 5.5 hours. The reaction was diluted with EA (15 ml) and washed with brine (10 ml). The organic layer was dried over Na₂SO₄ and concentrated to dryness. The resulting residue was purified by prep-TLC with DCM:MeOH (15:1) and then further purified by Prep-HPLC (Method A) to give the titled compound (15 mg). ¹H NMR (400 MHz, DMSO-d) δ 8.17 (s, 1H), 7.98 (s, 1H), 7.58 (dd, J=8.8, 5.6 Hz, 2H), 7.12 (t, J=8.9 Hz, 2H), 5.62 (s, 2H), 5.37 (s, 1H), 4.45 (s, 1H), 3.63-3.35 (m, 3H), 3.27 (s, 3H), 2.94 (d, J=9.4 Hz, 1H), 2.23 (dd, J=12.0, 2.8 Hz, 1H), 1.39 (s, 6H), 1.08 (d, J=6.3 Hz, 3H), 1.02 (d, J=6.4 Hz, 3H) ppm. MS: M/e 437 (M+1)⁺.

### Compound A298: 2-(7-(2,5-dimethyl-4-(methyl(3-methylquinoxalin-6-yl)amino)piperidin-1-yl)-4-methyl-5-oxo-4,5-dihydro-2H-pyrazolo[4,3-b]pyridin-2-yl)acetonitrile

### Step A: tert-butyl 2,5-dimethyl-4-((3-methylquinoxalin-6-yl)amino)piperidine-1-carboxylate

To a solution of 7-bromo-2-methylquinoxaline (440 mg, 2 mmol), tert-butyl 4-amino-2,5-dimethylpiperidine-1-carboxylate (550 mg, 2.4 mmol) and tBuONa (400 mg, 4 mmol) in toluene (20 mL) was added tBuXPhos (170 mg, 0.4 mmol) and tBuXPhos-Pd G3 (160 mg, 0.2 mmol). The mixture was stirred at 100° C. under N₂ for 16 hours. The reaction was cooled to room temperature, diluted with water, extracted with EA (100 mL×3), washed with brine, dried over Na₂SO₄, filtered, and concentrated to dryness. The resulting residue was purified by flash column chromatography to give the titled compound (300 mg, 40%). MS: M/e 371 (M+1)⁺.

### Step B: tert-butyl 2,5-dimethyl-4-(methyl(3-methylquinoxalin-6-yl)amino)piperidine-1-carboxylate

To a solution of tert-butyl 2,5-dimethyl-4-((3-methylquinoxalin-6-yl)amino)piperidine-1-carboxylate (36 mg, 0.1 mmol) in DMF (1 mL) at 0° C. was added NaH (8 mg, 60%, 0.2 mmol). After 15 min, a solution of CH₃I (21 mg, 0.15 mmol) in DMF (0.5 mL) was added. The reaction mixture was stirred at RT ˜80° C. for 16 hours. The reaction was cooled to room temperature, diluted with water, extracted with EA (60 mL), washed with brine, dried over Na₂SO₄, filtered, and concentrated to dryness. The resulting residue was purified by prep-TLC (PE/EA=1/1) to give titled compound (5 mg, 13%). MS: M/e 385 (M+1)⁺.

### Step C: N-(2,5-dimethylpiperidin-4-yl)-N,3-dimethylquinoxalin-6-amine

To a solution of tert-butyl 2,5-dimethyl-4-(methyl(3-methylquinoxalin-6-yl)amino)piperidine-1-carboxylate (8 mg) in DCM (1 mL) was added TFA (0.2 ml). The mixture was stirred at RT for 4 hours. The mixture was concentrated to dryness The resulting residue (10 m, crude) was used in the next step directly without further purification. MS: M/e 285 (M+1)⁺.

### Step D: 7-(2,5-dimethyl-4-(methyl(3-methylquinoxalin-6-yl)amino)piperidin-1-yl)-4-methyl-2-(tetrahydro-2H-pyran-2-yl)-2,4-dihydro-5H-pyrazolo[4,3-b]pyridin-5-one

To a solution of N-(2,5-dimethylpiperidin-4-yl)-N,3-dimethylquinoxalin-6-amine (10 mg, crude) in acetonitrile (2 mL) was added Intermediate 2 (8 mg, 0.02 mmol) and DIPEA (25 mg, 0.2 mmol). The resulting mixture was stirred at 100° C. overnight in a sealed tube. The mixture was concentrated to dryness. The reaction was quenched with water, extracted with EtOAc (60 mL), washed with brine, dried over Na₂SO₄, filtered, and concentrated to dryness. The resulting residue was purified by Prep-TLC(EA/PE=1/1) to give the titled compound (5 mg, 45% for two steps). MS: M/e 516 (M+1)⁺.

### Step E: 7-(2,5-dimethyl-4-(methyl(3-methylquinoxalin-6-yl)amino)piperidin-1-yl)-4-methyl-2,4-dihydro-5H-pyrazolo[4,3-b]pyridin-5-one

To a solution of 7-(2,5-dimethyl-4-(methyl(3-methylquinoxalin-6-yl)amino)piperidin-1-yl)-4-methyl-2-(tetrahydro-2H-pyran-2-yl)-2,4-dihydro-5H-pyrazolo[4,3-b]pyridin-5-one (5 mg) in DCM (1 mL) was added TFA (3 ml). The reaction was stirred at room temperature for 16 hours. The solvent was removed under reduced pressure. The resulting residue (6 mg, crude) was used directly for next step without further purification. MS: M/e 432 (M+1)⁺.

### Step F: 2-(7-(2,5-dimethyl-4-(methyl(3-methylquinoxalin-6-yl)amino)piperidin-1-yl)-4-methyl-5-oxo-4,5-dihydro-2H-pyrazolo[4,3-b]pyridin-2-yl)acetonitrile

To a solution of 7-(2,5-dimethyl-4-(methyl(3-methylquinoxalin-6-yl)amino)piperidin-1-yl)-4-methyl-2,4-dihydro-5H-pyrazolo[4,3-b]pyridin-5-one (6 mg, crude) in DMF (1 mL) was added potassium carbonate (27 mg, 0.2 mmol) and 2-chloroacetonitrile (3 mg, 0.03 mmol). The resulting mixture was stirred at room temperature overnight. The reaction mixture was diluted with water, extracted with EtOAc (50 mL), washed with brine, dried over Na₂SO₄, filtered, and concentrated to dryness. The crude was purified by Prep-TLC (DCM/MeOH=20/1) to give the titled compound (0.3 mg, 6.5% for two steps). ¹H NMR (400 MHz, CD₃OD) δ 8.42 (s, 1H), 7.96 (s, 1H), 7.85 (d, J=9.2 Hz, 1H), 7.62 (dd, J=2.8, 9.2 Hz, 1H), 7.09 (d, J=2.4 Hz, 1H), 5.67 (s, 1H), 5.51 (s, 2H), 4.24-4.12 (m, 1H), 3.64-3.58 (m, 0.5H), 3.46 (s, 3H), 3.19-3.06 (m, 1.5H), 2.96 (s, 3H), 2.65 (s, 3H), 2.27-2.15 (m, 2H), 2.06-1.98 (m, 0.5H), 1.79-1.71 (m, 1H), 1.64-1.56 (m, 0.5H), 1.44 (d, J=6.4 Hz, 3H), 0.99 (d, J=6.8 Hz, 3H) ppm. MS: M/e 471 (M+1)⁺.

### Compound A301: 2-allyl-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one

To a solution of 7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one (86 mg, 0.2 mmol) in DMF (2 mL) was added 1N LiOH (0.2 mL) and 3-bromoprop-1-ene (48 mg, 0.4 mmol). The reaction was stirred at room temperature for 16 hours. The reaction was diluted with water, extracted with EA (60 mL×2), washed with brine, dried over Na₂SO₄, filtered and concentrated to dryness. The resulting residue was purified by Prep-HPLC (Method A) to give the titled product (28 mg, 29%). ¹HNMR (400 MHz, CD₃OD) δ 8.79 (s, 1H), 8.06 (d, J=8.8 Hz, 1H), 7.99 (s, 1H), 7.94 (d, J=8.4 Hz, 1H), 7.79 (s, 1H), 6.15-5.95 (m, 1H), 5.53 (s, 1H), 5.31-5.19 (m, 2H), 5.01-4.88 (m, 1H), 4.87 (s, 2H), 4.36-4.20 (m, 1H), 3.99-3.85 (m, 1H), 3.50-3.43 (m, 1H), 3.43 (s, 3H), 3.14-3.02 (m, 1H), 2.97-2.86 (m, 2H), 2.77 (s, 3H), 1.48-1.39 (m, 6H), 1.03 (d, J=6.4 Hz, 3H) ppm. MS: M/e 472 (M+1)⁺.

### Compound A302: 2-(but-2-en-1-yl)-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one

To a solution of 7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one (86 mg, 0.2 mmol)(it was prepared according to the procedures as described for Compound A269d in stepG) in DMAc (2 mL) was added 1N LiOH (0.2 mL) and 1-chlorobut-2-ene (18 mg, 0.2 mmol). The reaction was stirred at room temperature for 16 hours. The reaction was diluted with water, extracted with EA (50 mL×2), washed with brine, dried over Na₂SO₄, filtered and concentrated to dryness. The resulting residue was purified by Prep-HPLC (Method A) to give the titled compound (40 mg, 41%) as a single diastereoisomer. ¹HNMR (400 MHz, CD₃OD) δ 8.79 (s, 1H), 8.06 (d, J=8.8 Hz, 1H), 7.98 (s, 1H), 7.94 (d, J=8.4 Hz, 1H), 7.79-7.73 (m, 1H), 5.91-5.65 (m, 2H), 5.52 (s, 1H), 5.02-4.89 (m, 1.5H), 4.82-4.76 (m, 1.5H), 4.42-4.15 (m, 1H), 3.99-3.85 (m, 1H), 3.50-3.43 (m, 1H), 3.43 (s, 3H), 3.14-3.02 (m, 1H), 2.97-2.86 (m, 2H), 2.77 (s, 3H), 1.83-1.70 (m, 3H), 1.50-1.35 (m, 6H), 1.03 (d, J=5.6 Hz, 3H) ppm. MS: M/e 486 (M+1)⁺.

### Compound A303: 2-(3,3-difluoroallyl)-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one

To a solution of 7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one (86 mg, 0.2 mmol) (it was prepared according to the procedures as described for Compound A269d in stepG) and Cs₂CO₃ (130 mg, 0.4 mmol) in CH₃CN (4 mL) was added 3-bromo-3,3-difluoroprop-1-ene (32 mg, 0.22 mmol). The reaction was stirred at room temperature for 56 hours. The reaction was diluted with water, extracted with EA (60 mL×2), washed with brine, dried over Na₂SO₄, filtered and concentrated to dryness. The resulting residue was purified by Prep-HPLC (Method A) to give the titled compound (12 mg, 41%) as a single diastereoisomer. ¹HNMR (400 MHz, CD₃OD) δ 8.79 (s, 1H), 8.06 (d, J=8.4 Hz, 1H), 7.99 (s, 1H), 7.94 (d, J=8.4 Hz, 1H), 7.84 (s, 1H), 5.53 (s, 1H), 4.89-4.78 (m, 2H), 4.63 (s, 2H), 4.45-4.20 (m, 1H), 4.00-3.85 (m, 1H), 3.52-3.42 (m, 1H), 3.43 (s, 3H), 3.11-3.02 (m, 1H), 2.98-2.84 (m, 2H), 2.77 (s, 3H), 1.48-1.36 (m, 6H), 1.04 (d, J=6.0 Hz, 3H) ppm. MS: M/e 508 (M+1)⁺.

### Compound A304: 2-(7-((2S,5R)-2,5-dimethyl-4-(1-(3-methylisoquinolin-6-yl)ethyl)piperazin-1-yl)-4-methyl-5-oxo-4,5-dihydro-2H-pyrazolo[4,3-b]pyridin-2-yl)acetonitrile

A solution of Intermediate 10 (50 mg, 0.17 mmol), 1-(3-methylisoquinolin-6-yl)ethan-1-ol (31 mg, 0.17 mmol), (cyanomethyl)trimethylphosphonium iodide (121 mg, 0.5 mmol) and DIPEA (108 mg, 0.83 mmol) in MeCN (3 ml) was stirred at 100° C. overnight. After completed, the solution was concentrated under reduced pressure. The resulting residue was purified Prep-HPLC (Method A) to give the titled compound (28 mg, 36%) as a mixture of diastereomers. ¹H NMR (400 MHz, CD₃OD) δ 9.09 (d, J=4.6 Hz, 1H), 8.08-8.00 (m, 1H), 7.92 (d, J=1.7 Hz, 1H), 7.81-7.75 (m, 2H), 7.63 (d, J=10.8 Hz, 1H), 5.56 (s, 1H), 5.46 (d, J=3.4 Hz, 2H), 4.60 (s, 1H), 3.90-3.73 (m, 1H), 3.70-3.64 (m, 1H), 3.46 (s, 1H), 3.43 (s, 3H), 3.19-2.95 (m, 1H), 2.95-2.77 (m, 2H), 2.66 (d, J=2.5 Hz, 3H), 1.44 (d, J=6.3 Hz, 3H), 1.35-1.20 (m, 3H), 1.20-1.02 (m, 3H). MS: M/e 470 (M+1)⁺

### Compound A309: 2-(7-((2S,5R)-4-(1-(3-(hydroxymethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 7-bromo-2-(bromomethyl)quinoxaline

A solution of 7-bromo-2-methylquinoxaline (2 g, 9.0 mmol), NBS (1.6 g, 9.0 mmol), AIBN (148 mg, 0.9 mmol) in CCl₄ (30 mL) was stirred at 90° C. overnight. The mixture was diluted with CH₂Cl₂ (20 mL), then washed with H₂O, brine, dried over Na₂SO₄ and concentrated to dryness. The resulting residue was purified by flash column chromatography to give the titled compound (1.9 g, 70%). MS: M/e 301 (M+1)⁺.

### Step B: (7-bromoquinoxaline-2-yl)methyl acetate

A solution of 7-bromo-2-(bromomethyl)quinoxaline (2 g, 9.0 mmol), 18-Crown-6 (1.6 g, 9.0 mmol), KOAc (148 mg, 0.9 mmol) in CH₃CN (30 mL) was stirred at RT for 2 h. The mixture was diluted with CH₂Cl₂ (20 mL), then washed with H₂O, brine, dried over Na₂SO₄ and concentrated to dryness. The resulting residue was purified by flash column chromatography to give the titled compound (1.3 g, 74%). MS: M/e 281 (M+1)⁺.

### Step C: (7-acetylquinoxalin-2-yl)methyl acetate

A mixture of (7-bromoquinoxalin-2-yl)methyl acetate (1.3 g, 4.64 mmol), tributyl(1-ethoxyvinyl)stannane (2.01 g, 5.57 mmol) and Pd(PPh₃)₂Cl₂ (325 mg, 0.46 mmol) in toluene (10 mL) was stirred at 100° C. under N₂ overnight. The solution was added HCl (0.3 mL, 4 M in 1,4-dioxane) and the mixture was stirred at room temperature for 0.5 hour. The reaction mixture was diluted with EA and concentrated to dryness. The resulting residue was purified by flash column chromatography to give the titled compound (800 mg, 71%). MS: M/e 245 (M+1)⁺.

### Step D: 1-(3-(hydroxymethyl)quinoxalin-6-yl)ethan-1-one

To a stirred solution of (7-acetylquinoxalin-2-yl)methyl acetate (800 mg, 3.27 mmol) in MeOH (10 mL) was added K₂CO₃ (905 mg, 6.56 mmol) at r.t. After then, the mixture was stirred for 2 hour. The mixture was diluted with CH₂Cl₂ (20 mL), then washed with H₂O, brine, dried over Na₂SO₄ and concentrated to dryness. The resulting residue was purified by flash column chromatography to give the titled compound (550 mg, 83%). MS: M/e 203 (M+1)⁺.

### Step E: 1-(3-(((tert-butyldimethylsilyl)oxy)methyl)quinoxalin-6-yl)ethan-1-one

To a stirred solution of 1-(3-(hydroxymethyl)quinoxalin-6-yl)ethan-1-one (550 mg, 2.72 mmol) and imidazole (278 mg, 4.08 mmol) in DCM (20 mL) was added TBDMSCl (449 mg, 2.99 mmol). After then, the mixture was stirred for 3 h. The mixture was diluted with CH₂Cl₂ (20 mL), then washed with H₂O, brine, dried over Na₂SO₄ and concentrated to dryness. The resulting residue was purified by flash column chromatography to give the titled compound (700 mg, 81%). MS: M/e 317 (M+1)⁺.

### Step F: 1-(3-(((tert-butyldimethylsilyl)oxy)methyl)quinoxalin-6-yl)ethan-1-ol

To a stirred solution of 1-(3-(((tert-butyldimethylsilyl)oxy)methyl)quinoxalin-6-yl)ethan-1-one (700 mg, 2.21 mmol) in MeOH (10 mL) was added NaBH₄ (84 mg, 2.21 mmol). After then, the mixture was stirred for 10 min. The reaction mixture was quenched with aq·NH₄Cl and extracted with CH₂Cl₂/IPA (3/1, 10 mL×3). The combined organic layers were washed with brine, dried over Na₂SO₄ and concentrated to give the titled compound (650 mg, 92%). MS: M/e 319 (M+1)⁺.

### Step G: 2-(7-((2S,5R)-4-(1-(3-(((tert-butyldimethylsilyl)oxy)methyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of Intermediate (400 mg, 1.34 mmol) in CH₃CN (5 mL) and was added 1-(3-(((tert-butyldimethylsilyl)oxy)methyl)quinoxalin-6-yl)ethan-1-ol (424 mg, 1.34 mmol), (cyanomethyl)trimethylphosphonium iodide (968 mg, 4 mmol) and DIPEA (860 mg, 6.66 mmol). The resulting mixture was stirred at 105° C. overnight. The mixture was diluted with CH₂Cl₂ (20 mL), then washed with H₂O, brine, dried over Na₂SO₄ and concentrated to dryness. The resulting residue was purified by flash column chromatography to give the titled compound (520 mg, 65%). MS: M/e 601 (M+1)⁺.

### Step H: 2-(7-((2S,5R)-4-(1-(3-(hydroxymethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 2-(7-((2S,5R)-4-(1-(3-(((tert-butyldimethylsilyl)oxy)methyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile (400 mg, 0.67 mmol) in THF (5 mL) was added TBAF (0.5 mL, 0.5 mmol). The reaction mixture was stirred at room for 3 hours. The reaction mixture was diluted with water and extracted with EtOAc (80 mL×2). The combined organic layers were washed with brine, dried over Na₂SO₄, filtered and concentrated to dryness. The resulting residue was purified by Prep-HPLC (Method A) to give the titled Compound A309 (254 mg, 78%), which was further separated into Compound A309a (35 mg) and Compound A309b (41 mg) by chiral Prep-HPLC. The chiral separation conditions are shown below.

| Column | CHIRAL Cellulose-SB | |
|---|---|---|
| Column Size | 2 cm × 25 cm, 5 um | |
| Mobile Phase A | MtBE | |
| Mobile Phase B | MeOH:DCM | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A309: ¹H NMR (400 MHz, CD₃OD) δ 9.00 (d, J=4.0 Hz, 1H), 8.13-8.02 (m, 2H), 7.98 (d, J=8.7 Hz, 1H), 7.93 (d, J=1.8 Hz, 1H), 5.57 (s, 1H), 5.47 (d, J=3.7 Hz, 2H), 4.94 (d, J=2.6 Hz, 2H), 4.61 (s, 1H), 4.00-3.80 (m, 1H), 3.69 (d, J=9.4 Hz, 1H), 3.48 (s, 1H), 3.43 (s, 3H), 3.16-2.85 (m, 2H), 2.85-2.62 (m, 1H), 1.51-1.22 (m, 6H), 1.22-1.01 (m, 3H). MS: M/e 487 (M+1)⁺.

Compound A309a (the earlier peak): ¹H NMR (400 MHz, CD₃OD) δ 8.99 (s, 1H), 8.10-8.02 (m, 2H), 7.98 (d, J=8.3 Hz, 1H), 7.93 (s, 1H), 5.57 (s, 1H), 5.47 (s, 2H), 4.94 (s, 2H), 4.62 (s, 2H), 3.82 (d, J=6.4 Hz, 1H), 3.69 (d, J=12.6 Hz, 2H), 3.44 (s, 3H), 2.87 (d, J=11.9 Hz, 1H), 2.22 (d, J=11.6 Hz, 1H), 1.46 (d, J=6.5 Hz, 3H), 1.25-1.19 (m, 6H). MS: M/e 487 (M+1)⁺.

Compound A309b (the later peak): ¹H NMR (400 MHz, CD₃OD) δ 9.00 (s, 1H), 8.10 (d, J=8.6 Hz, 1H), 8.05 (s, 1H), 7.99 (d, J=8.5 Hz, 1H), 7.92 (s, 1H), 5.57 (s, 1H), 5.46 (s, 2H), 4.95 (s, 2H), 4.27 (s, 1H), 3.96 (d, J=6.4 Hz, 1H), 3.43 (s, 4H), 3.08 (d, J=11.2 Hz, 1H), 2.93 (d, J=11.9 Hz, 2H), 1.44 (t, J=6.0 Hz, 7H), 1.05 (d, J=6.3 Hz, 3H). MS: M/e 487 (M+1)⁺

### Compound A310: 2-(7-((2S,5R)-4-(1-(3-cyclopropylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 7-bromo-2-cyclopropylquinoxaline

7-bromo-2-chloroquinoxaline (2.43 g, 10 mol) and Ferric acetylacetonate (176 mg, 0.5 mmol) were dissolved in dry THF (30 mL). A cyclopropylmagnesium bromide (1M in THF) solution (11 ml, 11 mmol) was added dropwise at 0° C. After stirred for 2 hour, the reaction mixture was quenched with saturated aqueous NH₄Cl solution and extracted with EtOAc. The organic layer was washed with brine, dried over Na₂SO₄, filtered and the solvent was removed under vacuum. The resulting residue was purified by column flash column chromatography to give the titled compound (1.2 g, 48%). MS: M/e 249 (M+1)⁺.

### Step B: 1-(3-cyclopropylquinoxalin-6-yl)ethan-1-one

A mixture of 7-bromo-2-cyclopropylquinoxaline (1.2 g, 4.8 mmol), tributyl(1-ethoxyvinyl)stannane (5.2 g, 14.5 mmol) and Pd(PPh₃)₂Cl₂ (336 mg, 0.048 mmol) in toluene (50 mL) was stirred at 100° C. under N₂ overnight. Then to the solution was added HCl (5 mL, 4 M in 1,4-dioxane) in drops and the mixture was stirred at room temperature for 30 minutes. The reaction mixture was diluted with EtOAc, washed with brine, dried over Na₂SO₄ and concentrated to dryness. The resulting residue was purified by flash column chromatography to give the titled compound (300 mg, 29%). MS: M/e 213 (M+1)⁺.

### Step C: 1-(3-cyclopropylquinoxalin-6-yl)ethan-1-ol

To a solution of 1-(3-cyclopropylquinoxalin-6-yl)ethan-1-one (300 mg, 1.42 mmol) in EtOH (10 mL) was added NaBH₄ (27 mg, 0.71 mmol) at 0° C. and the resulting mixture was stirred at room temperature for 4 hours. The reaction mixture was concentrated to dryness. The resulting residue was treated with water, extracted with DCM. The combined organic layers were washed with brine, dried over Na₂SO₄ and concentrated to dryness. The resulting residue was purified by flash column chromatography to give the titled compound (230 mg, 77%). MS: M/e 215 (M+1)⁺.

### Step D: tert-butyl (2S,5R)-4-(1-(3-cyclopropylquinoxalin-6-yl)ethyl)-2,5-dimethylpiperazine-1-carboxylate

A mixture of 1-(3-cyclopropylquinoxalin-6-yl)ethan-1-ol (214 mg, 1 mmol), tert-butyl (2S,5R)-2,5-dimethylpiperazine-1-carboxylate (257 mg, 1.2 mmol), (cyanomethyl)trimethylphosphonium iodide (363 mg, 1.5 mmol) and DIPEA (516 mg, 4 mmol) in MeCN (4 mL) was stirred at 100° C. overnight. The mixture was concentrated to dryness. The resulting residue was purified by flash column chromatography to give the titled compound (330 mg, 80%). MS: M/e 411 (M+1)⁺.

### Step E: 2-cyclopropyl-7-(1-((2R,5S)-2,5-dimethylpiperazin-1-yl)ethyl)quinoxaline

To a stirred solution of tert-butyl (2S,5R)-4-(1-(3-cyclopropylquinoxalin-6-yl)ethyl)-2,5-dimethylpiperazine-1-carboxylate (330 mg) in CH₂Cl₂ (5 mL) was added TFA (2 mL). The resulting mixture was stirred at room temperature for 2 hours. The reaction mixture was concentrated to dryness. The resulting residue (150 mg, crude) was used in the next step directly without further purification. MS: M/e 311 (M+1)⁺.

### Step F: 7-((2S,5R)-4-(1-(3-cyclopropylquinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

A mixture of 2-cyclopropyl-7-(1-((2R,5S)-2,5-dimethylpiperazin-1-yl)ethyl)quinoxaline (150 mg, 0.48 mmol), Intermediate 2 (280 mg, 0.72 mmol) and DIPEA (309 mg, 2.4 mmol) in DMAc (4 mL) was stirred at 100° C. overnight. The mixture was diluted with EtOAc (20 mL), washed with brine (10 mL×3), dried, concentrated. The resulting residue was purified by flash column chromatography to give the titled compound (200 mg, 77%). MS: M/e 542 (M+1)⁺.

### Step G: 7-((2S,5R)-4-(1-(3-cyclopropylquinoxalin-6-yl)ethyl)-2,5-dimethylpiperazin-1-yl)-4-methyl-2,4-dihydro-5H-pyrazolo[4,3-b]pyridin-5-one

To a solution of 7-((2S,5R)-4-(1-(3-cyclopropylquinoxalin-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (200 mg) in MeOH (3 mL) was added HCl (g) (4 M in dioxane, 3 ml). The reaction was stirred at room temperature for 4 hours. The solvent was removed under reduced pressure. The resulting residue (150 mg, crude) was used directly for next step without further purification. MS: M/e 458 (M+1)⁺.

### Step H: 2-(7-((2S,5R)-4-(1-(3-cyclopropylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 7-((2S,5R)-4-(1-(3-cyclopropylquinoxalin-6-yl)ethyl)-2,5-dimethylpiperazin-1-yl)-4-methyl-2,4-dihydro-5H-pyrazolo[4,3-b]pyridin-5-one (84 mg, 0.18 mmol) and K₂CO₃ (74 mg, 0.54 mmol) in DMF (2 mL) was added 2-iodoacetonitrile (34 mg, 0.20 mmol). The resulting mixture was stirred at room temperature overnight. The mixture was diluted with H₂O (10 mL), extracted with EtOAc (5 mL×3). The combined organic layers were washed with brine (10 mL), dried over Na₂SO₄, concentrated to dryness. The resulting residue was purified by Prep-TLC (CH₂Cl₂/MeOH=13/1) to give the titled compound (2 mg, 2%). ¹H NMR (400 MHz, CD₃OD) δ 8.75 (d, J=7.7 Hz, 1H), 8.03-7.96 (m, 1H), 7.89 (dd, J=16.8, 10.2 Hz, 3H), 5.56 (s, 1H), 5.47 (d, J=3.1 Hz, 2H), 3.98-3.70 (m, 1H), 3.68 (d, J=12.3 Hz, 1H), 3.45 (d, J=16.8 Hz, 4H), 3.15-3.05 (m, 1H), 2.91 (s, 1H), 2.83 (s, 1H), 2.42-2.18 (m, 2H), 1.43 (dd, J=10.9, 6.6 Hz, 5H), 1.24-1.17 (m, 6H), 1.04 (d, J=6.6 Hz, 2H) ppm. MS: M/e 497 (M+1)⁺.

Compound A311: 2-(7-((2S,5R)-4-(1-(3-(difluoromethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile.

### Step A: 7-bromo-2-vinylquinoline

To a solution of 7-bromo-2-chloroquinoxaline (3.0 g, 12.32 mmol), tributyl(vinyl)stannane (4.3 g, 13.55 mmol) and Pd(PPh₃)₄ (2.85 g, 2.464 mmol) in toluene (50 mL) was degassed 3 times under N₂ atmosphere.

The reaction was stirred 90° C. for 12 hours. The reaction mixture was diluted with water, extracted with EA (60 mL×2), washed with brine, dried over Na₂SO₄, filtered, and concentrated to dryness. The resulting residue was purified by flash column chromatography to give the titled compound (1.5 g, 52%). MS: M/e 235 (M+1)⁺

### Step B: 7-bromoquinoxaline-2-carbaldehyde

To a solution of 7-bromo-2-vinylquinoline (1.4 g, 5.983 mmol) in THF (30 mL) and H₂O (30 mL) was added O₅O₄ (76 mg, 0.300 mmol), followed NaIO₄ (3.84 g, 17.950 mmol). The mixture solution was stirred at room temperature for 24 hours. The reaction mixture was diluted with water, extracted with EA (60 mL×2), washed with brine, dried over Na₂SO₄, filtered, and concentrated to dryness. The resulting residue was purified by flash column chromatography (DCM:MeOH=20:1) to give the titled compound (1.0 g, 71%). MS: M/e 237 (M+1)⁺

### Step C: 7-bromo-2-(difluoromethyl)quinoxaline

To a solution of 7-bromoquinoxaline-2-carbaldehyde (1.0 g, 4.237 mmol) in DCM (50 mL) at 0° C. was added DAST (2.05 g, 12.712 mmol). The mixture was stirred at 0° C. for 2 hours. The reaction mixture was quenched with water and extracted with DCM (45 mL×2). The combined organic layers were washed with brine, dried over Na₂SO₄, filtered and concentrated to dryness. The resulting residue was purified by flash column chromatography (PE:EA=5:1) to give the titled compound (660 mg, 60%). MS: M/e 259 (M+1)⁺

### Step D: 1-(3-(difluoromethyl)quinoxalin-6-yl)ethan-1-one

To a solution of 7-bromo-2-(difluoromethyl)quinoxaline (660 mg, 2.558 mmol), tributyl(1-ethoxyvinyl)stannane (1.85 g, 5.116 mmol), Pd(PPh₃)₂Cl₂ (360 mg, 0.512 mmol) in toluene (35 mL) was stirred at 90° C. under N₂ for 4 hours. The reaction mixture was quenched with saturated NaHCO₃ aq·(20 mL), extracted with EA (30 mL×2), combined, washed brine (20 mL×2), dried and concentrated to dryness. The resulting oil was diluted with THF (20 mL). Then to the solution was added HCl (4 mL, 4 M in 1,4-dioxane) in drops and the mixture was stirred at room temperature For 1 hour. The reaction mixture was diluted with EA (30 mL), treated with saturated NaHCO₃ aq. to pH-8, washed with brine (20 mL×3), dried over Na₂SO₄ and concentrated to dryness. The resulting residue was purified by flash column chromatography (PE:EA=8:1) to give the titled compound (500 mg, 88%). MS: M/e 223 (M+1)⁺

### Step E: 1-(3-(difluoromethyl)quinoxalin-6-yl)ethan-1-ol

To a solution of 1-(3-(difluoromethyl)quinoxalin-6-yl)ethan-1-one (500 mg, 2.251 mmol) in CH₃OH (10 mL) was added NaBH₄ (86 mg, 2.252 mmol). The reaction was stirred at room temperature for 1 hours. The reaction mixture was quenched with saturated NH₄Cl, extracted with EA (30 mL×2), washed with brine, dried over Na₂SO₄, filtered, and concentrated to dryness. The resulting residue was purified by flash column chromatography (PE:EA=5:1) to give the titled compound (448 mg, 89%). MS: M/e 225 (M+1)⁺

### Step F: 2-(7-((2S,5R)-4-(1-(3-(difluoromethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

A solution of Intermediate 10 (80 mg, 0.267 mmol), 1-(3-(difluoromethyl)quinoxalin-6-yl)ethan-1-ol (120 mg, 0.533 mmol), (cyanomethyl)trimethylphosphonium iodide (130 mg. 0.533 mmol) and DIPEA (103 mg, 0.801 mmol) in CH₃CN (2 ml). The mixture solution was degassed 3 times under N₂ atmosphere. Then the mixture solution was stirred at 105° C. for 24 hours. The reaction mixture was concentrated under reduced pressure. The resulting residue was purified by Prep-TLC (DCM:MeOH=15:1) and Prep-HPLC (Method A) to give the titled compound (25 mg, 19%). ¹H NMR (400 MHz, CD₃OD) δ 9.13 (d, J=4.3 Hz, 1H), 8.23-8.09 (m, 3H), 7.93 (d, J=1.8 Hz, 1H), 6.99 (td, J=54.5, 3.6 Hz, 1H), 5.57 (s, 1H), 5.47 (d, J=3.7 Hz, 2H), 4.89-4.86 (m, 0.5H), 4.67-4.23 (m, 1.5H), 4.00 (q, J=6.4 Hz, 0.5H), 3.85 (q, J=6.5 Hz, 0.5H), 3.74-3.64 (m, 1H), 3.52-3.40 (m, 3.5H), 3.09 (dd, J=11.8, 3.9 Hz, 0.5H), 3.00-2.81 (m, 1.5H), 2.21 (d, J=12.3 Hz, 0.5H), 1.52-1.39 (m, 4.5H), 1.22 (t, J=7.1 Hz, 3H), 1.06 (d, J=6.5 Hz, 1.5H) ppm. MS: M/e 507 (M+1)⁺.

### Compound A312: 2-(7-((2S,5R)-4-(1-(3-(1,1-difluoroethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 1-(7-bromoquinoxalin-2-yl)ethan-1-one

To a solution of 7-bromo-2-chloroquinoxaline (1 g, 4 mol) in toluene (10 mL) was added tributyl(1-ethoxyvinyl)stannane (1.8 g, 5 mol) and Pd(PPh₃)₄ (701 mg, 1 mmol). The reaction mixture was protected by N₂ atmosphere and stirred at 100° C. overnight. The mixture was cooled down to rt, added HCl/Dioxane (4M, 10 ml) and stirred at room temperature for 30 mins. The mixture was concentrated in vacuo. The residue was added H₂O and adjusted pH 7-8 by NaHCO₃ aqueous solution. The resulting mixture was extracted with EA, and then concentrated by using a rotary evaporator, to give a residue. The resulting residue was purified by flash column chromatography to give the titled compound (500 mg, 50%). M/e 251 (M+1)⁺.

### Step B: 7-bromo-2-(1,1-difluoroethyl)quinoxaline

To a solution of 1-(7-bromoquinoxalin-2-yl)ethan-1-one (186 mg, 0.74 mmol) in DAST (2 ml) were added MeOH (1 drop). The mixture was sealed and stirred at 80° C. overnight. The reaction was diluted with DCM and washed with water. The organic layer was separated, dried by Na₂SO₄, filtered, and concentrated to dryness. The resulting residue was purified by flash column chromatography to give the titled compound (170 mg, 85%). MS: M/e 273 (M+1)⁺.

### Step C: 1-(3-(1,1-difluoroethyl)quinoxalin-6-yl)ethan-1-one

To a solution of 7-bromo-2-(1,1-difluoroethyl)quinoxaline (170 mg, 0.625 mol) in toluene (10 mL) was added tributyl(1-ethoxyvinyl)stannane (361 mg, 1 mol) and Pd(PPh₃)₄ (140 mg, 0.2 mmol). The reaction mixture was protected by N₂ atmosphere and stirred at 100° C. overnight. The mixture was cooled down to RT, added HCl/1,4-dioxane (4M, 10 ml) and stirred at room temperature for 30 mins. The mixture was concentrated in vacuo. The residue was added H₂O and adjusted pH 7-8 by NaHCO₃ aqueous solution. The resulting mixture was extracted with EA, and then concentrated by using a rotary evaporator, to give a residue. The resulting residue was purified by flash column chromatography to give the titled compound (100 mg, 68%). M/e 237 (M+1)⁺.

### Step D: 1-(3-(1,1-difluoroethyl)quinoxalin-6-yl)ethan-1-ol

NaBH₄ (38 mg, 1 mmol) was added to 1-(3-(1,1-difluoroethyl)quinoxalin-6-yl)ethan-1-one (100 mg, 0.42 mol) in EtOH (20 ml) at 0° C. for 1 hour. The reaction was quenched by adding water. The mixture was extracted with EtOAc and washed with brine. The organic layer was separated, dried by Na₂SO₄, filtered and concentrated to dryness. The resulting residue was purified by flash column chromatography to give the titled compound (50 mg, 50%). MS: M/e 239 (M+1)⁺.

### Step D: 2-(7-((2S,5R)-4-(1-(3-(1,1-difluoroethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 1-(3-(1,1-difluoroethyl)quinoxalin-6-yl)ethan-1-ol (50 mg, 0.2 mmol), Intermediate 10 (40 mg, 0.13 mmol) and (cyanomethyl)trimethylphosphonium iodide (120 mg, 1 mmol) in CH₃CN (5 mL) was added DIPEA (250 mg, 2 mmol). The mixture solution was degassed 3 times under N₂ atmosphere. Then the mixture solution was stirred at 105° C. for 24 hours. The reaction was quenched with saturated NH₄Cl (20 mL) at room temperature. The resulting mixture was extracted with EA (35 mL×2). The combined organic layers were dried over Na₂SO₄ and concentrated under reduced pressure. The resulting residue was purified by Prep-HPLC (Method A) to the titled compound (10.68 mg, 15.9%). ¹H NMR (400 MHz, CD₃OD) δ 9.24 (d, J=3.9 Hz, 1H), 8.25-8.06 (m, 3H), 7.99 (d, J=0.6 Hz, 1H), 5.61 (d, J=1.2 Hz, 2H), 5.41 (d, J=5.3 Hz, 1H), 4.48 (s, 1H), 4.04-3.92 (m, 1H), 3.83 (d, J=6.5 Hz, 1H), 3.55 (d, J=12.7 Hz, 1H), 3.28 (s, 3H), 3.01-2.94 (m, 1H), 2.86-2.67 (m, 2H), 2.21 (d, J=4.6 Hz, 1H), 2.16 (d, J=4.6 Hz, 1H), 2.12 (d, J=4.6 Hz, 1H), 1.38 (dd, J=10.9, 6.5 Hz, 3H), 1.31 (d, J=6.5 Hz, 1.5H), 1.11 (dd, J=9.5, 6.5 Hz, 3H), 0.98 (d, J=6.5 Hz, 1.5H). MS: M/e 521 (M+1)⁺.

### Compound A313: 2-(7-((2S,5R)-2,5-dimethyl-4-(1-(3-(methyl-d₃)quinoxalin-6-yl)ethyl)piperazin-1-yl)-4-methyl-5-oxo-4,5-dihydro-2H-pyrazolo[4,3-b]pyridin-2-yl)acetonitrile

### Step A: 7-bromo-2-(methyl-d3)quinoxaline

A solution of 7-bromo-2-methylquinoxaline (850 mg, 3.81 mmol) and PhCOOH (465 mg, 3.81 mmol) in D₂O (4 ml) and DMF (8 ml) was stirred at 150° C. for 2 days. The solution was diluted with EA (20 ml) and washed with brine (10 ml×2). The organic layer was dried over Na₂SO₄ and concentrated to dryness. The resulting residue was purified by flash column chromatography with 0-15% EA in PE to give the titled compound (700 mg, 81%). ¹H NMR (400 MHz, DMSO-d₆) δ 8.90 (s, 1H), 8.23 (d, J=2.0 Hz, 1H), 8.01 (d, J=8.9 Hz, 1H), 7.92 (dd, J=8.9, 2.1 Hz, 1H) ppm.

### Step B: 1-(3-(methyl-d₃)quinoxalin-6-yl)ethan-1-one

A solution of 7-bromo-2-(methyl-d₃)quinoxaline (300 mg, 1.33 mmol), tributyl(1-ethoxyvinyl)stannane (719 mg, 1.99 mmol) and Pd(PPh₃)₂Cl₂ (93 mg, 0.13 mmol) in toluene. (6 ml) was stirred at 100° C. overnight. The mixture was cooled to 0° C. HCl (4M in dioxane, 2 ml) was added to the above solution and stirred at 0° C. for 20 min. The solution was diluted with EA (15 ml) and then washed with brine (10 ml). The organic layer was dried over Na₂SO₄ and concentrated to dryness. The resulting residue was purified by flash column chromatography with 10-30% EA in PE to give the titled compound (200 mg, 79%). MS: M/e 190 (M+1)⁺.

### Step C: 1-(3-(methyl-d₃)quinoxalin-6-yl)ethan-1-ol

A solution of 1-(3-(methyl-d₃)quinoxalin-6-yl)ethan-1-one (200 mg, 1.05 mmol) and NaBH₄ (20 mg, 0.53 mmol) in EtOH (7 ml) was stirred at 0° C. for 10 min. The solution was diluted with EA (20 ml) and then washed with brine (10 ml×2). The organic layer was dried over Na₂SO₄ and concentrated to dryness. The resulting residue was purified by flash column chromatography with 20-60% EA in PE to give the titled compound (200 mg, 100%). MS: M/e 192 (M+1)⁺.

### Step D: 2-(7-((2S,5R)-2,5-dimethyl-4-(1-(3-(methyl-d₃)quinoxalin-6-yl)ethyl)piperazin-1-yl)-4-methyl-5-oxo-4,5-dihydro-2H-pyrazolo[4,3-b]pyridin-2-yl)acetonitrile

A solution of Intermediate 10 (222 mg, 0.74 mmol), 1-(3-(methyl-d₃)quinoxalin-6-yl)ethan-1-ol (200 mg, 1.05 mmol), (cyanomethyl)trimethylphosphonium iodide (542 mg, 2.23 mmol) and DIPEA (958 mg, 7.43 mmol) in CH₃CN (6 ml) was stirred at 100° C. overnight. The reaction was diluted with EtOAc (20 ml) and washed with brine (10 ml). The organic layer was concentrated under reduced pressure. The resulting residue was purified by flash column chromatography with 0-10% MeOH in DCM to give the titled Compound A313, which was further separated into Compound A313a (17 mg) and Compound A313b (10 mg) by chiral Prep-HPLC. The chiral separation conditions are shown below.

| Column | CHIRAL Cellulose-SB | |
|---|---|---|
| Column Size | 2 cm × 25 cm, 5 um | |
| Mobile Phase A | MtBE | |
| Mobile Phase B | Hex:EtOH | |
| Flow Rate | 20 | mL/min |
| Wave Length | UV 220 nm | |
| Temperature | 25° | C. |

| | |
|---|---|
| Prep-HPLC Equipment | Prep-HPLC-Gilson |

Compound A313: ¹H NMR (400 MHz, DMSO-d₆) δ 8.82 (d, J=5.3 Hz, 1H), 8.20 (s, 0.15H), 8.08-7.98 (m, 2H), 7.94 (d, J=11.7 Hz, 1H), 7.86 (t, J=7.9 Hz, 1H), 5.62 (s, 2H), 5.41 (d, J=6.7 Hz, 1H), 4.48 (s, 1H), 3.89 (q, J=6.3 Hz, 0.5H), 3.75 (q, J=6.6 Hz, 0.5H), 3.60-3.50 (m, 1H), 3.34 (s, 4H), 2.98-2.92 (m, 0.5H), 2.89-2.64 (m, 2H), 2.10 (d, J=11.7 Hz, 0.5H), 1.38 (t, J=6.5 Hz, 3H), 1.30 (d, J=6.5 Hz, 1H), 1.11 (t, J=5.6 Hz, 3.5H), 0.96 (d, J=6.4 Hz, 1.5H) ppm. MS: M/e 474 (M+1)⁺

Compound A313a (the earlier peak): ¹H NMR (400 MHz, DMSO-d₆) δ 8.81 (s, 1H), 8.02 (d, J=8.7 Hz, 1H), 7.99 (s, 1H), 7.93 (s, 1H), 7.85 (d, J=8.4 Hz, 1H), 5.62 (s, 2H), 5.41 (s, 1H), 3.76 (q, J=6.4 Hz, 1H), 3.62-3.50 (m, 2H), 3.33-3.32 (m, 2H), 3.28 (s, 3H), 2.73 (d, J=8.3 Hz, 1H), 2.10 (d, J=11.5 Hz, 1H), 1.37 (d, J=6.5 Hz, 3H), 1.11 (t, J=5.6 Hz, 6H) ppm. MS: M/e 474 (M+1)⁺.

Compound A313b (the later peak): ¹H NMR (400 MHz, DMSO-d₆) δ 8.83 (s, 1H), 8.05 (d, J=8.6 Hz, 1H), 8.00 (s, 1H), 7.96 (s, 1H), 7.87 (d, J=8.6 Hz, 1H), 5.62 (s, 2H), 5.40 (s, 1H), 3.89 (q, J=6.6 Hz, 1H), 3.33-3.32 (m, 2H), 3.28 (s, 3H), 3.26 (s, 1H), 2.95 (d, J=7.7 Hz, 1H), 2.86-2.75 (m, 2H), 1.35 (d, J=6.4 Hz, 3H), 1.30 (d, J=6.5 Hz, 3H), 0.96 (d, J=6.5 Hz, 3H) ppm. MS: M/e 474 (M+1)⁺.

### Compound A314: 2-(7-((2S,5R)-2,5-dimethyl-4-(1-(3-(trifluoromethyl)quinoxalin-6-yl)ethyl)piperazin-1-yl)-4-methyl-5-oxo-4,5-dihydro-2H-pyrazolo[4,3-b]pyridin-2-yl)acetonitrile

### Step A: mixture of 7-bromo-2-(trifluoromethyl)quinoxaline and 6-bromo-2-(trifluoromethyl)quinoxaline

To a solution of 3,3-dibromo-1,1,1-trifluoropropan-2-one (5.77 g, 21.4 mmol) and CH₃COONa (8.44 g, 106.9 mmol) in CH₃OH (60 mL) and H₂O (60 mL) was stirred at 90° C. for 30 mins. Then 4-bromobenzene-1,2-diamine (2.0 g, 10.7 mmol) was added. The mixture was stirred at room temperature for 12 hours. After filtered, the mixture was extracted with EA (35 mL×2), washed with brine, dried over Na₂SO₄, filtered, and concentrated to dryness. The resulting residue was purified by flash column chromatography (PE:EA=5:1) to give the titled compound (3.2 g). The titled compound (1.6 g) was further separated into 6-bromo-2-(trifluoromethyl)quinoxaline (620 mg, the earlier peak) and 7-bromo-2-(trifluoromethyl)quinoxaline (680 mg, the later peak) by chiral Prep-SFC. The chiral separation conditions are shown below.

| Column | CHIRALPAK IG | |
|---|---|---|
| Column Size | 3 cm × 25 cm, 5 um | |
| Mobile Phase A | CO₂ | |
| Mobile Phase B | MeOH | |
| Flow Rate | 100 | mL/min |
| Wave Length | UV 220 nm | |
| Temperature | 25° | C. |

| | |
|---|---|
| Prep-SFC Equipment | Prep-SFC-150 |

mixture of 7-bromo-2-(trifluoromethyl)quinoxaline and 6-bromo-2-(trifluoromethyl)quinoxaline: ¹H NMR (400 MHz, CDCl₃) δ 9.18 (s, 1H), 8.47-8.38 (m, 1H), 8.10 (d, J=9.0 Hz, 1H), 8.00 (t, J=7.7 Hz, 1H).

6-bromo-2-(trifluoromethyl)quinoxaline (the earlier peak): ¹H NMR (400 MHz, CDCl3) δ 9.18 (s, 1H), 8.42 (d, J=2.0 Hz, 1H), 8.11 (d, J=9.0 Hz, 1H), 7.99 (dd, J=9.0, 2.0 Hz, 1H) ppm. MS: M/e 277 (M+1)⁺

7-bromo-2-(trifluoromethyl)quinoxaline (the later peak): ¹H NMR (400 MHz, CDCl₃) δ 9.19 (s, 1H), 8.43 (d, J=1.8 Hz, 1H), 8.10 (d, J=9.0 Hz, 1H), 8.01 (dd, J=9.0, 1.9 Hz, 1H). MS: M/e 277 (M+1)⁺

### Step B: 1-(3-(trifluoromethyl)quinoxalin-6-yl)ethan-1-one

To a solution of 7-bromo-2-(trifluoromethyl)quinoxaline (360 mg, 1.304 mmol), tributyl(1-ethoxyvinyl)stannane (942 mg, 2.609 mmol), Pd(PPh₃)₂Cl₂ (183 mg, 0.261 mmol) in toluene (20 mL) was stirred at 90° C. under N₂ for 12 hours. The reaction mixture was quenched with saturated NaHCO₃ aq·(30 mL), extracted with EA (50 mL×2), combined, washed brine (30 mL×2), dried and concentrated to dryness. The resulting oil was diluted with THF (30 mL). Then to the solution was added HCl (3 mL, 4 M in 1,4-dioxane) in drops and the mixture was stirred at room temperature For 1 hour. The reaction mixture was diluted with EA (30 mL), treated with saturated NaHCO₃ aq. to pH-8, washed with brine (30 mL×3), dried over Na₂SO₄ and concentrated to dryness. The resulting residue was purified by flash column chromatography (PE:EA=6:1) to give the titled compound (300 mg, 96%). MS: M/e 241 (M+1)⁺

### Step C: 1-(3-(trifluoromethyl)quinoxalin-6-yl)ethan-1-ol

To a solution of 1-(3-(trifluoromethyl)quinoxalin-6-yl)ethan-1-one (300 mg, 1.25 mmol) in CH₃OH (15 mL) was added NaBH₄ (48 mg, 1.25 mmol). The reaction was stirred at room temperature for 1 hours. The reaction mixture was quenched with saturated NH₄Cl, extracted with EA (35 mL×2), washed with brine, dried over Na₂SO₄, filtered, and concentrated to dryness. The resulting residue was purified by flash column chromatography (PE:EA=5:1) to give the titled compound (290 mg, 96%). MS: M/e 243 (M+1)⁺

### Step D: 2-(7-((2S,5R)-2,5-dimethyl-4-(1-(3-(trifluoromethyl)quinoxalin-6-yl)ethyl)piperazin-1-yl)-4-methyl-5-oxo-4,5-dihydro-2H-pyrazolo[4,3-b]pyridin-2-yl)acetonitrile

A solution of Intermediate 10 (70 mg, 0.233 mmol), 1-(3-(trifluoromethyl)quinoxalin-6-yl)ethan-1-ol (85 mg, 0.358 mmol), (cyanomethyl)trimethylphosphonium iodide (113 mg. 0.466 mmol) and DIPEA (90 mg, 0.699 mmol) in CH₃CN (1 ml). The mixture solution was degassed 3 times under N₂ atmosphere. Then the mixture solution was stirred at 105° C. for 24 hours. The reaction mixture was concentrated under reduced pressure. The resulting residue was purified by Prep-TLC (DCM:MeOH=15:1) and further purified by Prep-HPLC (Method A) to give the titled compound (17 mg, 14%). ¹H NMR (400 MHz, CD₃OD) δ 9.23 (d, J=4.7 Hz, 1H), 8.33-8.15 (m, 3H), 7.93 (d, J=0.9 Hz, 1H), 5.57 (s, 1H), 5.47 (d, J=3.6 Hz, 2H), 4.98-4.88 (m, 0.5H), 4.73-4.27 (m, 1.5H), 4.03 (q, J=6.5 Hz, 0.5H), 3.88 (q, J=6.4 Hz, 0.5H), 3.76-3.65 (m, 1H), 3.52-3.48 (m, 0.5H), 3.47 (s, 3H), 3.09 (dd, J=11.6, 3.8 Hz, 0.5H), 2.97-2.84 (m, 1.5H), 2.20 (d, J=12.7 Hz, 0.5H), 1.47 (dd, J=12.9, 6.5 Hz, 4.5H), 1.23 (t, J=6.5 Hz, 3H), 1.07 (d, J=6.5 Hz, 1.5H) ppm. MS: M/e 525 (M+1)⁺.

### Compound A315: 2-(7-((2S,5R)-2,5-dimethyl-4-(1-(2-(trifluoromethyl)quinoxalin-6-yl)ethyl)piperazin-1-yl)-4-methyl-5-oxo-4,5-dihydro-2H-pyrazolo[4,3-b]pyridin-2-yl)acetonitrile

### Step A: 1-(2-(trifluoromethyl)quinoxalin-6-yl)ethan-1-one

To a solution of 6-bromo-2-(trifluoromethyl)quinoxaline (440 mg, 1.594 mmol), tributyl(1-ethoxyvinyl)stannane (1.15 g, 3.189 mmol), Pd(PPh₃)₂Cl₂ (224 mg, 0.319 mmol) in toluene (25 mL) was stirred at 90° C. under N₂ for 12 hours. The reaction mixture was quenched with saturated NaHCO₃ aq·(30 mL), extracted with EA (50 mL×2), combined, washed brine (30 mL×2), dried and concentrated to dryness. The resulting oil was diluted with THF (30 mL). Then to the solution was added HCl (3 mL, 4 M in 1,4-dioxane) in drops and the mixture was stirred at room temperature for 1 hour. The reaction mixture was diluted with EtOAc (30 mL), treated with saturated NaHCO₃ aq. to pH-8, washed with brine (30 mL×3), dried over Na₂SO₄ and concentrated to dryness. The resulting residue was purified by flash column chromatography (PE:EA=6:1) to give the titled compound (360 mg, 94%). MS: M/e 241 (M+1)⁺

### Step B: 1-(2-(trifluoromethyl)quinoxalin-6-yl)ethan-1-ol

To a solution of 1-(2-(trifluoromethyl)quinoxalin-6-yl)ethan-1-one (360 mg, 1.5 mmol) in CH₃OH (15 mL) was added NaBH₄ (57 mg, 1.5 mmol). The reaction was stirred at room temperature for 1 hours. The reaction mixture was quenched with saturated NH₄Cl, extracted with EA (35 mL×2), washed with brine, dried over Na₂SO₄, filtered, and concentrated to dryness. The resulting residue was purified by flash column chromatography (PE:EA=5:1) to give the titled compound (300 mg, 83%). MS: M/e 243 (M+1)⁺

### Step C: 2-(7-((2S,5R)-2,5-dimethyl-4-(1-(2-(trifluoromethyl)quinoxalin-6-yl)ethyl)piperazin-1-yl)-4-methyl-5-oxo-4,5-dihydro-2H-pyrazolo[4,3-b]pyridin-2-yl)acetonitrile

A solution of 2-(7-((2S,5R)-2,5-dimethylpiperazin-1-yl)-4-methyl-5-oxo-4,5-dihydro-2H-pyrazolo[4,3-b]pyridin-2-yl)acetonitrile (70 mg, 0.233 mmol), 1-(2-(trifluoromethyl)quinoxalin-6-yl)ethan-1-ol (85 mg, 0.358 mmol), (cyanomethyl)trimethylphosphonium iodide (113 mg. 0.466 mmol) and DIPEA (90 mg, 0.699 mmol) in CH₃CN (1 ml). The mixture solution was degassed 3 times under N₂ atmosphere. Then the mixture solution was stirred at 105° C. for 24 hours. The reaction mixture was concentrated under reduced pressure.

The resulting residue was purified by Prep-TLC (DCM:MeOH=15:1) and Prep-HPLC (Method A) to give the titled compound (19 mg, 16%). ¹H NMR (400 MHz, CD₃OD) δ 9.25 (d, J=3.6 Hz, 1H), 8.29-8.13 (m, 3H), 7.93 (d, J=1.5 Hz, 1H), 5.57 (s, 1H), 5.47 (d, J=3.7 Hz, 2H), 4.98-4.92 (m, 0.5H), 4.68-4.30 (m, 1.5H), 4.03 (d, J=6.5 Hz, 0.5H), 3.88 (d, J=6.5 Hz, 0.5H), 3.70 (d, J=10.3 Hz, 1H), 3.52-2.43 (m, 0.5H), 3.47 (s, 3H), 3.13-3.05 (m, 0.5H), 2.98-2.85 (m, 1.5H), 2.25-2.19 (m, 0.5H), 1.47 (dd, J=12.9, 6.5 Hz, 4.5H), 1.23 (t, J=6.9 Hz, 3H), 1.07 (d, J=6.5 Hz, 1.5H) ppm. MS: M/e 525 (M+1)⁺.

### Compound A316: 2-(7-((2S,5R)-4-(1-(2-hydroxy-3-methylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 6-bromo-2-methoxy-3-methylquinoxaline

To a solution of 6-bromo-2-chloro-3-methylquinoxaline (740 mg, 2.89 mmol) in MeOH (20 mL) was added CH₃ONa (312 mg, 5.78 mmol) at room temperature. The reaction mixture was stirred at 60° C. overnight. The solvent was removed under reduced pressure. The resulting residue was purified by flash column chromatography to give the titled compound (700 mg, 96%). MS: M/e 253 (M+1)⁺.

### Step B: 1-(2-methoxy-3-methylquinoxalin-6-yl)ethan-1-one

A mixture of 6-bromo-2-methoxy-3-methylquinoxaline (700 mg, 2.78 mmol), tributyl (1-ethoxyvinyl)stannane (1.2 g, 3.33 mmol) and Pd(PPh₃)₂Cl₂ (175 mg, 0.25 mmol) in toluene (10 mL) was stirred at 100° C. under N₂ for 4 hours. The reaction mixture was quenched with saturated NaHCO₃ aq·(50 mL), extracted with EA (25 mL×3), combined, washed brine (25 mL×3), dried and concentrated to dryness. The resulting oil was diluted with THF (20 mL). Then to the solution was added HCl (6 mL, 4 M in 1,4-dioxane) in drops and the mixture was stirred at room temperature For 1 hour. The reaction mixture was diluted with EA (50 mL), treated with saturated NaHCO₃ aq. to pH-8, washed with brine (20 mL×3), dried over Na₂SO₄ and concentrated to dryness. The resulting residue was purified by flash column chromatography to give the titled compound (420 mg, 70%). MS: M/e 217 (M+1)⁺.

### Step C: 1-(2-methoxy-3-methylquinoxalin-6-yl)ethan-1-ol

To a solution of 1-(2-methoxy-3-methylquinoxalin-6-yl)ethan-1-one (420 mg, 1.9 mmol) in MeOH (10 mL) was added NaBH₄ (72 mg, 1.9 mmol) at room temperature and the resulting mixture was stirred at room temperature for 2 hours. The reaction mixture was concentrated to dryness. The resulting residue was treated with saturated NaHCO₃ aq·, extracted with EA (20 mL×3). The combined organic layers were washed with brine (20 mL×3), dried over Na₂SO₄ and concentrated to dryness. The resulting residue was purified by flash column chromatography to give the titled compound (320 mg, 76%). MS: M/e 219 (M+1)⁺.

### Step D: 2-(7-((2S,5R)-4-(1-(2-methoxy-3-methylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 1-(2-methoxy-3-methylquinoxalin-6-yl)ethan-1-ol (280 mg, 1.28 mmol), 2-(7-((2S,5R)-2,5-dimethylpiperazin-1-yl)-4-methyl-5-oxo-4,5-dihydro-2H-pyrazolo[4,3-b]pyridin-2-yl)acetonitrile (257 mg, 0.85 mmol) and (cyanomethyl)trimethylphosphonium iodide (311 mg, 1.28 mmol) in CH₃CN (5 mL) was added DIPEA (548 mg, 4.25 mmol). The mixture was sealed in a bottle and heated at 100° C. for 16 hours. The mixture was cooled to room temperature, diluted with water, extracted with EA (50 mL×2), washed with brine, dried over Na₂SO₄, filtered, and concentrated to dryness. The resulting residue was purified by flash column chromatography (DCM:MeOH=15:1) to give the titled compound (95 mg, 22%). MS: M/e 501 (M+1)⁺.

### Step E: 2-(7-((2S,5R)-4-(1-(2-hydroxy-3-methylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 2-(7-((2S,5R)-4-(1-(2-methoxy-3-methylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile (55 mg, 0.11 mmol) in DCM (2 mL) was added BBr₃ (1.1 mL, 1.1 mmol, 1 M in DCM). The resulting mixture was stirred at 0° C. for 2 hours. n-Hexane was added to the mixture. A yellow solid was precipitated, then filtered. The resulting solid was dissolved in saturated aq·NaHCO₃ (5 mL) and extracted with DCM (10 mL). The organic layers were concentrated, and The resulting residue was purified by Prep-HPLC (Method A) to give the titled compound (1.52 mg, 3%). ¹H NMR (400 MHz, DMSO-d₆) δ 7.99 (s, 1H), 7.61-7.58 (m, 1H), 7.45-7.43 (m, 1H), 7.24-7.19 (m, 1H), 5.62 (s, 2H), 5.41-5.39 (m, 1H), 3.65-3.51 (m, 3H), 3.27 (s, 3H), 2.78-2.69 (m, 2H), 2.33 (s, 3H), 2.15-2.13 (m, 1H), 1.26-1.24 (m, 5H), 1.12-1.05 (m, 4H), 0.92-0.91 (m, 2H) ppm. MS: M/e 487 (M+1)⁺.

### Compound A317: 2-(7-((2S,5R)-2,5-dimethyl-4-((S)-1-(3-methylquinoxalin-6-yl)ethyl)piperazin-1-yl)-6-fluoro-4-methyl-5-oxo-4,5-dihydro-2H-pyrazolo[4,3-b]pyridin-2-yl)acetonitrile

To a mixture of 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile (47 mg, 0.1 mmol, Compound A269d) in CH₃CN (2 mL) was added Select F reagent (53 mg, 0.15 mmol). The mixture was stirred at room temperature for 6 hours. The reaction was diluted with water, extracted with DCM (60 mL×2), washed with brine, dried over Na₂SO₄, filtered, and concentrated to dryness. The resulting residue was purified by Prep-TLC (DCM:MeOH=15:1) to give the titled compound (17 mg, 34%) as a single diastereoisomer. ¹H NMR (40-MHz, CD₃OD) δ 8.79 (s, 1H), 8.06 (d, J=8.8 Hz, 1H), 8.02-7.91 (m, 3H), 5.46 (s, 2H), 4.80-4.67 (m, 1H), 4.07-3.95 (m, 1H), 3.83-3.74 (m, 1H), 3.69-3.61 (m, 1H), 3.49 (s, 3H), 3.20-3.12 (m, 1H), 2.84-2.75 (m, 2H), 2.77 (s, 3H), 1.51-1.40 (m, 6H), 1.08 (d, J=6.4 Hz, 3H) ppm. MS: M/e 489 (M+1)⁺.

### Compound A318: 2-(cyanomethyl)-7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridine-6-carbonitrile

### Step A: 2-(6-bromo-7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile

To a mixture of 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile (47 mg, 0.1 mmol, Compound A269d) (47 mg, 0.1 mmol) in CH₃CN (2 mL) was added a solution of NBS (18 mg, 0.1 mmol) in CH₃CN (1 mL). The mixture was stirred at room temperature for 4 hours. The reaction was diluted with water, extracted with DCM (60 mL×2), washed with brine, dried over Na₂SO₄, filtered, and concentrated to dryness. The resulting residue was purified by Prep-TLC (DCM:MeOH=15:1) to give the titled compound (50 mg, 90%) as a single diastereoisomer. MS: M/e 549 (M+1)⁺.

### Step B: 2-(cyanomethyl)-7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridine-6-carbonitrile

To a mixture of 2-(6-bromo-7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile (50 mg, 0.09 mmol) in DMF (2 mL) was added Zn(CN)₂ (31 mg, 0.27 mmol) and Pd(PPh₃)₄ (31 mg, 0.027 mmol). The mixture was stirred at 100° C. for 16 hours under N₂. The reaction was cooled to room temperature, diluted with water, extracted with DCM (60 mL×2), washed with brine, dried over Na₂SO₄, filtered, and concentrated to dryness. The resulting residue was purified by Prep-TLC (DCM:MeOH=15:1) to give the titled compound (10 mg, 22%) as a single diastereoisomer. ¹H NMR (400 MHz, CD₃OD) δ 8.79 (s, 1H), 8.07 (d, J=8.4 Hz, 1H), 8.02-7.92 (m, 3H), 5.50 (s, 2H), 4.89-4.86 (m, 1H), 4.01-3.85 (m, 2H), 3.42 (s, 3H), 3.31-3.28 (m, 1H), 3.21-3.10 (m, 1H), 3.04-2.88 (m, 2H), 2.77 (s, 3H), 1.66 (d, J=6.4 Hz, 3H), 1.43 (d, J=6.4 Hz, 3H), 1.00 (d, J=6.0 Hz, 3H) ppm. MS: M/e 496 (M+1)⁺.

### Compound A319: 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-d]pyrimidin-2-yl)acetonitrile

### Step A: 7-((2S,5R)-2,5-dimethyl-4-((S)-1-(3-methylquinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one

To a solution of Intermediate 9 (200 mg, 0.8 mmol) in CH₃CN (10 mL) was added phosphorus oxychloride (135 mg, 0.88 mmol). The reaction mixture was stirred at room temperature for 1 hour. Then 7-((S)-1-((2R,5S)-2,5-dimethylpiperazin-1-yl)ethyl)-2-methylquinoxaline (273 mg, 0.96 mmol) and DIPEA (310 mg, 2.4 mmol) were added. The reaction mixture was stirred at room temperature for 2 hours. The reaction mixture was diluted with water and extracted with EA (80 mL×2). The combined organic layers were washed with brine, dried over Na₂SO₄, filtered and concentrated to dryness. The resulting residue was purified by flash column chromatography to give the titled compound (180 mg, 43%). MS: M/e 517 (M+1)⁺.

### Step B: 7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-d]pyrimidin-5-one

To a stirred solution of 7-((2S,5R)-2,5-dimethyl-4-((S)-1-(3-methylquinoxalin-6-yl)ethyl)piperazin-1-yl)-4-methyl-2-(tetrahydro-2H-pyran-2-yl)-2,4-dihydro-5H-pyrazolo[4,3-d]pyrimidin-5-one (180 mg) in MeOH(2 mL) was added HCl in 1,4-dioxane solution (2 mL, 8 mmol, 4M). The resulting mixture was stirred at room temperature for 2 hours. The reaction mixture was concentrated to dryness. The resulting residue (160 mg, crude) was used in the next step directly without further purification. MS: M/e 433 (M+1)⁺.

### Step C: 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-d]pyrimidin-2-yl)acetonitrile

To a solution of 7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-d]pyrimidin-5-one (120 mg, 0.28 mmol) and K₂CO₃ (76 mg, 0.56 mmol) in DMF (10 mL) was added 2-iodoacetonitrile (70 mg, 0.42 mmol). The reaction mixture was stirred at room temperature overnight. The reaction mixture was diluted with water and extracted with EA (80 mL×2). The combined organic layers were washed with brine, dried over Na₂SO₄, filtered and concentrated to dryness. The resulting residue was purified by flash column chromatography to give the titled compound (70 mg, 43%). ¹H NMR (400 MHz, CD₃OD) 8.79 (s, 1H), 8.06 (d, J=8.7 Hz, 1H), 7.98 (s, 1H), 7.96-7.87 (m, 2H), 6.01-5.87 (m, 0.5H), 5.55 (s, 1H), 5.45 (s, 1H), 5.36 (s, 1H), 4.76-4.64 (m, 0.5H), 3.93 (q, J=6.4 Hz, 1H), 3.75-3.70 (m, 0.5H), 3.40 (s, 3H), 3.37-3.35 (m, 0.5H), 3.03-2.90 (m, 3H), 2.77 (s, 3H), 1.56-1.54 (m, 3H), 1.43 (d, J=6.5 Hz, 3H), 0.97 (d, J=6.5 Hz, 3H). MS: M/e 472 (M+1)⁺.

### Compound A322: 2-(7-((2S,5R)-4-(1-(benzo[d]thiazol-5-yl)ethyl)-2,5-diethylpiperazin-1-yl)-4-methyl-5-oxo-4,5-dihydro-2H-pyrazolo[4,3-b]pyridin-2-yl)acetonitrile

### Step A: 1-(benzo[d]thiazol-5-yl)ethan-1-one

A mixture of 5-bromobenzo[d]thiazole (200 mg, 0.93 mmol), tributyl(1-ethoxyvinyl)stannane (677 mg, 1.86 mmol) and Pd(PPh₃)₂Cl₂ (66 mg, 0.09 mmol) in toluene (8 mL) was stirred at 100° C. under N₂ for 16 hours. The reaction mixture was quenched by HCl (1 mL, 4 M in 1,4-dioxane) in drops and the mixture was stirred at room temperature for 0.5 hour. The reaction mixture was concentrated. The resulting residue was purified by flash column chromatography to give the titled compound (60 mg, 36%). MS: M/e 178 (M+1)⁺.

### Step B: 1-(benzo[d]thiazol-5-yl)ethan-1-ol

To a solution of 1-(benzo[d]thiazol-5-yl)ethan-1-one (60 mg, 0.34 mmol) in MeOH (3 mL) was added NaBH₄ (13 mg, 0.34 mmol) at room temperature and the resulting mixture was stirred at room temperature for 5 min. The reaction mixture was diluted with DCM and washed with water, dried over Na₂SO₄ and concentrated to give the titled compound (70 mg). MS: M/e 180 (M+1)⁺.

### Step C: 2-(7-((2S,5R)-4-(1-(benzo[d]thiazol-5-yl)ethyl)-2,5-diethylpiperazin-1-yl)-4-methyl-5-oxo-4,5-dihydro-2H-pyrazolo[4,3-b]pyridin-2-yl)acetonitrile

To a solution of Intermediate 5 (50 mg, 0.15 mmol) in CH₃CN (2 mL) and was added 1-(benzo[d]thiazol-5-yl)ethan-1-ol (41 mg, 0.23 mmol), (cyanomethyl)trimethylphosphonium iodide (110 mg, 0.45 mmol) and DIPEA (197 mg, 1.5 mmol). The resulting mixture was stirred at 105° C. overnight. The reaction solvent was removed under reduce pressure, The reaction was diluted with DCM and washed with water. The organic layer was separated, dried by Na₂SO₄, filtered, and concentrated to dryness. The resulting residue was purified by flash column chromatography (DCM:MeOH) to give the titled compound (12 mg) ¹H NMR (400 MHz, CD₃OD) δ 9.25 (d, J=4.8 Hz, 1H), 8.15-8.00 (m, 2H), 7.92 (d, J=3.1 Hz, 1H), 7.60 (t, J=8.4 Hz, 1H), 5.56 (s, 1H), 5.46 (d, J=5.1 Hz, 2H), 4.83-4.18 (m, 1H), 4.02-3.52 (m, 2H), 3.43 (s, 3H), 3.26-2.66 (m, 3H), 2.49-2.33 (m, 1H), 2.26-1.53 (m, 4H), 1.41 (dd, J=12.6, 6.5 Hz, 3H), 1.01 (dt, J=27.8, 7.4 Hz, 3H), 0.62 (dt, J=42.4, 7.4 Hz, 3H)′ppm. MS: M/e 490 (M+1)⁺.

### Compound A323: 2-(7-((2S,5R)-4-(1-(3H-spiro[benzo[b][1,4]dioxine-2,1′-cyclopropan]-7-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 2-(benzyloxy)-5-bromobenzaldehyde

To a solution of 5-bromo-2-hydroxybenzaldehyde (10 g, 50 mol) in CH₃CN (100 mL) was added BnBr (10 g, 60 mol) and K₂CO₃ (13.9 g, 100 mmol). The reaction mixture was protected by N₂ atmosphere and stirred at 60° C. overnight. The mixture was cooled down to RT and added H₂O. The resulting mixture was extracted with EA, and then concentrated by using a rotary evaporator to give a residue. The resulting residue was purified by flash column chromatography to give the titled compound (13.36 g, 92%). ¹H NMR (400 MHz, CDCl₃) δ 10.45 (s, 1H), 7.94 (d, J=2.5 Hz, 1H), 7.59 (dd, J=8.8, 2.5 Hz, 1H), 7.45-7.31 (m, 6H), 6.94 (d, J=8.9 Hz, 1H), 5.17 (s, 2H). MS: M/e 291 (M+1)⁺.

### Step B: 2-(benzyloxy)-5-bromophenyl formate

To a solution of 2-(benzyloxy)-5-bromobenzaldehyde (13.36 g, 46 mol) in DCM (100 mL) was added m-CPBA (11.7 g, 69 mol). The reaction mixture was stirred at RT overnight. The mixture was added H₂O and extracted with EA. The organic phase was dried with Na₂SO₄ and concentrated by using a rotary evaporator to give a residue. The resulting residue was purified by flash column chromatography to give the titled compound (11.8 g, 84.2%). MS: M/e 307 (M+1)⁺.

### Step C: 2-(benzyloxy)-5-bromophenol

To a solution of 2-(benzyloxy)-5-bromophenyl formate (11.8 g, 44 mol) in MeOH (50 mL) and H₂O (50 ml) was added K₂CO₃ (9 g, 65 mol). The reaction mixture was stirred at RT for 4 h. The mixture was filtered, The filter cake was dried to give the titled compound (8 g, 62.5%). MS: M/e 279 (M+1)⁺.

### Step D: methyl 2-(2-(benzyloxy)-5-bromophenoxy)-4-bromobutanoate

To a solution of 2-(benzyloxy)-5-bromophenol (2.78 g, 10 mol) in DMF (30 mL) was added methyl 2,4-dibromobutanoate (5.2 g, 20 mol) and K₂CO₃ (5.6 g, 40 mmol). The reaction mixture was stirred at RT overnight. The mixture was added H₂O and extracted with EA. The organic phase was dried with Na₂SO₄ and concentrated by using a rotary evaporator to give a residue. The resulting residue was purified by flash column chromatography to give the titled compound (3.7 g, 82%). MS: M/e 457 (M+1)⁺.

### Step E: methyl 1-(2-(benzyloxy)-5-bromophenoxy)cyclopropane-1-carboxylate

To a solution of methyl 2-(2-(benzyloxy)-5-bromophenoxy)-4-bromobutanoate (3.7 g, 8.1 mol) in THF (15 mL) was added t-BuOK (1.12 g, 10 mol). The reaction mixture was stirred at RT overnight. The mixture was added H₂O and extracted with EA. The organic phase was dried with Na₂SO₄ and concentrated by using a rotary evaporator to give a residue. The resulting residue was purified by flash column chromatography to give the titled compound (2.5 g, 843.3%). ¹H NMR (400 MHz, CDCl₃) δ 7.40-7.31 (m, 5H), 7.05 (d, J=2.3 Hz, 1H), 6.98 (dd, J=8.6, 2.3 Hz, 1H), 6.75 (d, J=8.6 Hz, 1H), 5.10 (s, 2H), 3.73 (s, 3H), 1.63 (dd, J=8.4, 5.2 Hz, 2H), 1.37 (dd, J=8.4, 5.1 Hz, 2H). MS: M/e 377 (M+1)⁺.

### Step F: (1-(2-(benzyloxy)-5-bromophenoxy)cyclopropyl)methanol

To a solution of methyl 1-(2-(benzyloxy)-5-bromophenoxy)cyclopropane-1-carboxylate (800 mg, 2.11 mol) in THF (10 mL) was added LiAlH₄ (170 mg, 4.5 mol) at 0° C. The reaction mixture was stirred at RT for 1 h. The mixture was added H₂O (0.2 ml), 15% NaOH aqueous solution (0.2 ml) and H₂O (0.6 ml) and filtered. The filtrate was dried over Na₂SO₄ and concentrated in vacuo. The resulting residue was purified by flash column chromatography to give the titled compound (450 mg, 61.3%). MS: M/e 349 (M+1)⁺.

### Step G: 4-bromo-2-(1-(hydroxymethyl)cyclopropoxy)phenol

To a solution of (1-(2-(benzyloxy)-5-bromophenoxy)cyclopropyl)methanol (600 mg, 1.71 mol) in DCM (15 mL) was added TMSI (1M. 3.5 ml, 3.5 mol). The reaction mixture was stirred at RT for 1 h. The mixture was added H₂O and extracted with EA. The organic phase was dried with Na₂SO₄ and concentrated by using a rotary evaporator to give a residue. The resulting residue was purified by flash column chromatography to give the titled compound (300 mg, 68%). MS: M/e 259 (M+1)⁺.

### Step H: 7-bromo-3H-spiro[benzo[b][1,4]dioxine-2,1′-cyclopropane]

To a solution of 4-bromo-2-(1-(hydroxymethyl)cyclopropoxy)phenol (300 mg, 1.2 mol) in THF (15 mL) was added PPh₃ (365 mg, 1.4 mol) and DIAD (283 mg, 1.4 mmol). The reaction mixture was stirred at RT for 2 h. The mixture was added H₂O and extracted with EA. The organic phase was dried with Na₂SO₄ and concentrated by using a rotary evaporator to give a residue. The resulting residue was purified by flash column chromatography to give the titled compound (250 mg, 86.8%). MS: M/e 241 (M+1)⁺.

### Step I: 1-(3H-spiro[benzo[b][1,4]dioxine-2,1′-cyclopropan]-7-yl)ethan-1-one

To a solution of 7-bromo-3H-spiro[benzo[b][1,4]dioxine-2,1′-cyclopropane](250 mg, 1 mol) in toluene (10 mL) was added tributyl(1-ethoxyvinyl)stannane (541 mg, 1.5 mol) and Pd(PPh₃)₄ (140 mg, 0.2 mmol). The reaction mixture was protected by N₂ atmosphere and stirred at 100° C. overnight. The mixture was cooled down to rt, added HCl/Dioxane (4M, 10 ml) and stirred at room temperature for 30 mins. The mixture was concentrated in vacuo. The residue was added H₂O and adjusted pH 7-8 by NaHCO₃ aqueous solution. The resulting mixture was extracted with EA, and then concentrated by using a rotary evaporator, to give a residue. The resulting residue was purified by flash column chromatography to give the titled compound (120 mg, 58.8%). M/e 205 (M+1)⁺.

### Step J: 1-(3H-spiro[benzo[b][1,4]dioxine-2,1′-cyclopropan]-7-yl)ethan-1-ol

NaBH₄ (38 mg, 1 mmol) was added to 1-(3H-spiro[benzo[b][1,4]dioxine-2,1′-cyclopropan]-7-yl)ethan-1-one (120 mg, 0.58 mol) in EtOH (20 ml) at 0° C. for 1 hour. The reaction was quenched by adding water. The mixture was extracted with EA and washed with brine. The organic layer was separated, dried by Na₂SO₄, filtered and concentrated to dryness. The resulting residue was purified by flash column chromatography to give the titled compound (40 mg, 33.3%). MS: M/e 207 (M+1)⁺.

### Step K: 2-(7-((2S,5R)-4-(1-(3H-spiro[benzo[b][1,4]dioxine-2,1′-cyclopropan]-7-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 1-(3H-spiro[benzo[b][1,4]dioxine-2,1′-cyclopropan]-7-yl)ethan-1-ol (40 mg, 0.2 mmol), Intermediate 10 (30 mg, 0.1 mmol) and (cyanomethyl)trimethylphosphonium iodide (120 mg, 1 mmol) in CH₃CN (5 mL) was added DIPEA (250 mg, 2 mmol). The mixture solution was degassed 3 times under N₂ atmosphere. Then the mixture solution was stirred at 105° C. for 24 hours. The reaction was quenched with saturated NH₄Cl (20 mL) at room temperature. The resulting mixture was extracted with EA (35 mL×2). The combined organic layers were dried over Na₂SO₄ and concentrated under reduced pressure. The resulting residue was purified by Prep-HPLC (Method A) to give the titled compound (2.03 mg, 4.51%). ¹H NMR (400 MHz, CD₃OD) δ 7.92 (d, J=1.2 Hz, 1H), 6.87-6.79 (m, 3H), 5.55 (d, J=1.8 Hz, 1H), 5.47 (d, J=1.7 Hz, 2H), 4.57 (s, 1H), 4.28 (d, J=18.9 Hz, 1H), 4.15-4.09 (m, 2H), 3.55 (m, 2H), 3.45-3.36 (m, 4H), 2.95 (dd, J=11.6, 4.2 Hz, 1H), 2.80 (dd, J=11.8, 2.3 Hz, 0.5H), 2.70 (dd, J=12.1, 4.0 Hz, 0.5H), 1.37 (d, J=6.5 Hz, 2H), 1.32-1.27 (m, 3H), 1.21 (d, J=6.6 Hz, 1.5H), 1.11 (d, J=6.4 Hz, 1.5H), 1.03-0.96 (m, 3H), 0.85-0.80 (m, 2H). MS: M/e 489 (M+1)⁺.

### Compound A324: 2-(7-((2S,5R)-4-(1-(2,3-dihydro-[1,4]dioxino[2,3-b]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 2-(7-((2S,5R)-4-(1-(2,3-dihydro-[1,4]dioxino[2,3-b]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile

A solution of Intermediate 10 (50 mg, 0.17 mmol), 1-(2,3-dihydro-[1,4]dioxino[2,3-b]pyridin-6-yl)ethan-1-ol from Step D of Compound A159 (30 mg, 0.17 mmol), (cyanomethyl)trimethylphosphonium iodide (121 mg, 0.5 mmol) and DIPEA (108 mg, 0.83 mmol) in MeCN (3 ml) was stirred at 100° C. overnight. After completed, the solution was concentrated under reduced pressure. The resulting residue was purified Prep-HPLC (Method A) to give the titled compound (30 mg, 39%). ¹H NMR (400 MHz, CD₃OD) δ 7.92 (s, 1H), 7.68 (s, 1H), 7.36 (s, 1H), 5.56 (s, 1H), 5.47 (s, 2H), 4.68-4.50 (m, 1H), 4.46-4.40 (m, 2H), 4.30-4.25 (m, 2H), 3.72-3.61 (m, 1H), 3.61-3.55 (m, 1H), 3.54-3.46 (m, 1H), 3.43 (s, 3H), 3.42-3.32 (m, 1H), 3.14-2.85 (m, 1H), 2.83-2.76 (m, 1H), 1.36 (dd, J=13.3, 6.6 Hz, 3H), 1.33-1.18 (m, 3H), 1.16-0.98 (m, 3H). MS: M/e 464 (M+1)⁺

### Compound A325: 2-(7-((2S,5R)-4-(1-(3,3-dimethyl-2,3-dihydro-[1,4]dioxino[2,3-b]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 2-chloro-6-(hydroxymethyl)pyridin-3-ol

A solution of 2-chloropyridin-3-ol (2 g, 15.50 mmol), formaldehyde (37%, 12.6 g, 155 mmol), NaHCO₃ (3.9 g, 46.5 mmol) in water (50 mL) was stirred at 90° C. overnight. The reaction mixture was quenched with con. HCl and extracted with CH₂Cl₂/IPA (3/1, 10 mL×3). The combined organic layers were washed with brine, dried over Na₂SO₄, concentrated to give the titled compound (2.1 g, 85%). MS: M/e 160 (M+1)⁺.

### Step B: 1-((2-chloro-6-(hydroxymethyl)pyridin-3-yl)oxy)propan-2-one

A solution of 2-chloro-6-(hydroxymethyl)pyridin-3-ol (2.1 g, 13.21 mmol), 1-chloropropan-2-one (1.46 g, 15.85 mmol), KI (219 mg, 1.32 mmol) and K₂CO₃ (3.65 g, 26.42 mmol) in ACN (100 mL) was stirred at 60° C. for 1 h. The mixture was diluted with CH₂Cl₂ (20 mL), then washed with H₂O, brine, dried over Na₂SO₄, concentrated and purified by flash column chromatography to give the titled compound (2.7 g, 95%). MS: M/e 216 (M+1)⁺.

### Step C: 6-chloro-5-(2-oxopropoxy)picolinaldehyde

A mixture of 1-((2-chloro-6-(hydroxymethyl)pyridin-3-yl)oxy)propan-2-one (2.7 g, 12.56 mmol) and Dess-Martin Periodinane (6.4 g, 15.07 mmol) in DCM (30 mL) was stirred at r.t for 3 h. The reaction mixture was diluted with DCM and washed with water, concentrated to dryness. The resulting residue was purified by flash column chromatography to give the titled compound (2.6 g, 97%). MS: M/e 214 (M+1)⁺.

### Step D: 1-((2-chloro-6-(1-hydroxyethyl)pyridin-3-yl)oxy)-2-methylpropan-2-ol

To a stirred solution of 6-chloro-5-(2-oxopropoxy)picolinaldehyde (2.6 g, 12.21 mmol) in dry THF (200 mL) was added dropwise MeMgBr (1.0 M, 30.5 mL, 30.5 mmol) at 0° C. After then, the mixture was stirred for half an hour. The reaction was quenched with aq·NH₄Cl, extracted with EtOAc (100 mL×3). The combined organic layers were washed with brine, dried over Na₂SO₄, concentrated and purified by flash column chromatography to give the titled compound (2.6 g, 87%). MS: M/e 246 (M+1)⁺.

### Step E: 1-(3,3-dimethyl-2,3-dihydro-[1,4]dioxino[2,3-b]pyridin-6-yl)ethan-1-ol

A mixture of 1-((2-chloro-6-(1-hydroxyethyl)pyridin-3-yl)oxy)-2-methylpropan-2-ol (1 g, 4.08 mmol), Pd(OAc)₂ (91 mg, 0.41 mmol) BINAP (254 mg, 0.41 mmol) and Cs₂CO₃ (2.66 g, 8.16 mmol) in toluene (30 mL) was stirred at 100° C. under N₂ overnight. The reaction mixture was extracted with EtOAc (100 mL×3). The combined organic layers were washed with brine, dried over Na₂SO₄, concentrated and purified by flash column chromatography to give the titled compound (550 mg, 64%). MS: M/e 210 (M+1)⁺.

### Step F: 2-(7-((2S,5R)-4-(1-(3,3-dimethyl-2,3-dihydro-[1,4]dioxino[2,3-b]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile

A solution of Intermediate 10 (50 mg, 0.17 mmol), 1-(3,3-dimethyl-2,3-dihydro-[1,4]dioxino[2,3-b]pyridin-6-yl)ethan-1-ol (35 mg, 0.17 mmol), (cyanomethyl)trimethylphosphonium iodide (121 mg, 0.5 mmol) and DIPEA (108 mg, 0.83 mmol) in MeCN (3 ml) was stirred at 100° C. overnight. After completed, the solution was concentrated under reduced pressure. The resulting residue was purified by Prep-HPLC (Method A) to give the titled compound (28 mg, 34%). ¹H NMR (400 MHz, CD₃OD) δ 8.33 (s, 1H), 7.93 (d, J=3.2 Hz, 1H), 7.32 (dd, J=8.0, 4.9 Hz, 1H), 7.12 (d, J=8.0 Hz, 1H), 5.58 (d, J=5.1 Hz, 1H), 5.48 (d, J=2.0 Hz, 2H), 3.96 (s, 2H), 3.71-3.59 (m, 2H), 3.49 (d, J=9.6 Hz, 1H), 3.44 (s, 3H), 3.13-2.79 (m, 2H), 2.77-2.27 (m, 1H), 1.47-1.39 (m, 6H), 1.39-1.36 (m, 3H), 1.36-1.25 (m, 3H), 1.25-0.98 (m, 3H). MS: M/e 492 (M+1)⁺.

### Compound A326: 2-(7-((2S,5R)-4-(1-(2,2-dimethyl-2,3-dihydro-[1,4]dioxino[2,3-b]pyridin-7-yl)ethyl)-2,5-dimethylpiperazin-1-yl)-4-methyl-5-oxo-4,5-dihydro-2H-pyrazolo[4,3-b]pyridin-2-yl)acetonitrile

### Step A: methyl 2,2-dimethyl-2,3-dihydro-[1,4]dioxino[2,3-b]pyridine-7-carboxylate

A solution of methyl 6-chloro-5-hydroxynicotinate (2 g, 10.70 mmol), 2,2-dimethyloxirane (3.85 g, 53.48 mmol) in DMAc (30 mL) was stirred at 140° C. overnight. The mixture was diluted with CH₂Cl₂ (20 mL), then washed with H₂O, brine, dried over Na₂SO₄, concentrated and purified by flash column chromatography to give the titled compound (1.85 mg, 77%). MS: M/e 224 (M+1)⁺.

### Step B: 2,2-dimethyl-2,3-dihydro-[1,4]dioxino[2,3-b]pyridine-7-carboxylic acid

To a stirred solution of methyl 2,2-dimethyl-2,3-dihydro-[1,4]dioxino[2,3-b]pyridine-7-carboxylate (1.85 g, 0.58 mmol) in MeOH (10 mL) was added aq·NaOH (2.0 M, 2 mL). After then, the mixture was stirred for 2 hours. The mixture was concentrated to give the aqueous layer, which was acidified to pH=3-4 with aq·HCl, then extracted with CH₂Cl₂/IPA (3/1, 20 mL×4). The combined organic layers were washed with brine, dried over Na₂SO₄, concentrated to give the titled compound (1.67 g, 96.3%). MS: M/e 210 (M+1)⁺.

### Step C: N-methoxy-N,2,2-trimethyl-2,3-dihydro-[1,4]dioxino[2,3-b]pyridine-7-carboxamide

A mixture of 2,2-dimethyl-2,3-dihydro-[1,4]dioxino[2,3-b]pyridine-7-carboxylic acid (500 mg, 2.369 mmol), N,O-dimethylhydroxylamine hydrochloride (278 mg, 2.87 mmol), HATU (1.1 g, 2.87 mmol) and DIEPA (617 mg, 4.78 mmol) in CH₂Cl₂ (20 mL) was a stirred overnight. The reaction mixture was washed with brine, dried over Na₂SO₄, concentrated and purified by flash column chromatography to give the titled compound (550 mg, 91%). MS: M/e 253 (M+1)⁺.

### Step D: 1-(2,2-dimethyl-2,3-dihydro-[1,4]dioxino[2,3-b]pyridin-7-yl)ethan-1-one

To a stirred solution of N-methoxy-N,2,2-trimethyl-2,3-dihydro-[1,4]dioxino[2,3-b]pyridine-7-carboxamide (200 mg, 0.79 mmol) in dry THF (10 mL) was added dropwise MeMgBr (3.0 M, 0.26 mL, 0.79 mmol) at 0° C. After then, the mixture was stirred for half an hour. The reaction was quenched with aq·NH₄Cl, extracted with EtOAc (10 mL×3). The combined organic layers were washed with brine, dried over Na₂SO₄, concentrated and purified by flash column chromatography to give the titled compound (80 mg, 48%). MS: M/e 208 (M+1)⁺.

### Step E: 1-(2,2-dimethyl-2,3-dihydro-[1,4]dioxino[2,3-b]pyridin-7-yl)ethan-1-ol

To a stirred solution of 1-(2,2-dimethyl-2,3-dihydro-[1,4]dioxino[2,3-b]pyridin-7-yl)ethan-1-one (80 mg, 0.39 mmol) in MeOH (10 mL) was added NaBH₄ (29 mg, 0.77 mmol). After then, the mixture was stirred for 10 min. The reaction mixture was quenched with aq·NH₄Cl and extracted with CH₂Cl₂/IPA (3/1, 10 mL×3). The combined organic layers were washed with brine, dried over Na₂SO₄, concentrated to give the titled compound (50 mg, 62%). MS: M/e 210 (M+1)⁺.

### Step F: 2-(7-((2S,5R)-4-(1-(2,2-dimethyl-2,3-dihydro-[1,4]dioxino[2,3-b]pyridin-7-yl)ethyl)-2,5-dimethylpiperazin-1-yl)-4-methyl-5-oxo-4,5-dihydro-2H-pyrazolo[4,3-b]pyridin-2-yl)acetonitrile

A solution of Intermediate 10 (50 mg, 0.17 mmol), 1-(2,2-dimethyl-2,3-dihydro-[1,4]dioxino[2,3-b]pyridin-7-yl)ethan-1-ol (35 mg, 0.17 mmol), (cyanomethyl)trimethylphosphonium iodide (121 mg, 0.5 mmol) and DIPEA (108 mg, 0.83 mmol) in CH₃CN (3 ml) was stirred at 100° C. overnight. After completed, the solution was concentrated under reduced pressure. The resulting residue was purified by Prep-HPLC (Method A) to give the titled compound (40 mg, 49%). ¹H NMR (400 MHz, CD₃OD) δ 7.93 (s, 1H), 7.68 (s, 1H), 7.33 (s, 1H), 5.56 (s, 1H), 5.48 (s, 2H), 4.13 (d, J=3.6 Hz, 2H), 3.72-3.46 (m, 3H), 3.43 (s, 3H), 3.05-2.58 (m, 4H), 1.38 (d, J=6.2 Hz, 3H), 1.36-1.32 (m, 6H), 1.30-1.18 (m, 3H), 1.15-1.00 (m, 3H). MS: M/e 492 (M+1)⁺.

### Compound A328: 2-(7-((2S,5R)-4-(1-(2-(1-(difluoromethoxy)ethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile

### Step A: 1-(2-bromo-5-fluorophenyl)ethan-1-ol

To a solution of 2-bromo-5-fluorobenzaldehyde (2 g, 10 mmol) in THF (20 mL) at 0° C. was added methyl magnesium bromide (4 mL, 3M, 12 mol). The reaction mixture was stirred at 0° C. for 2 hours, stirred at room temperature for 2 hours. The reaction was quenched by adding aqueous NH₄Cl. The resulting mixture was extracted with EA, and then concentrated by using a rotary evaporator, to give a residue. The resulting residue was purified by flash column chromatography (PE:EA=5:1) to give the titled compound (1.8 g, 83%). MS: M/e 219 (M+1)⁺.

### Step B: 1-bromo-2-(1-(difluoromethoxy)ethyl)-4-fluorobenzene

To a solution of 1-(2-bromo-5-fluorophenyl)ethan-1-ol (1.6 g, 7.3 mmol), KOAc (2.8 g, 29.2 mmol) in CH₂Cl₂ (2.1 mL), H₂O (2.1 mL) were added TMSCF₂Br (2.9 g, 14.6 mmol) at room temperature. After being stirred at room temperature for 2 hours, the reaction mixture was diluted with CH₂Cl₂ (50 mL) and washed with brine. The organic layer was concentrated and purified by combi flash to give the title compound (280 mg, 14%).

### Step C: 1-(2-(1-(difluoromethoxy)ethyl)-4-fluorophenyl)ethan-1-one

A mixture of 1-bromo-2-(1-(difluoromethoxy)ethyl)-4-fluorobenzene (320 mg, 1.2 mmol), tributyl(1-ethoxyvinyl)stannane (500 mg, 1.4 mmol) and Pd(PPh₃)₂Cl₂ (70 mg, 0.1 mmol) in toluene (5 mL) was stirred at 100° C. under N₂ for 4 hours. The reaction mixture was quenched with saturated NaHCO₃ aq·(50 mL), extracted with EA (25 mL×3), combined, washed brine (25 mL×3), dried and concentrated to dryness. The resulting oil was diluted with THF (20 mL). Then to the solution was added HCl (2 mL, 4 M in 1,4-dioxane) in drops and the mixture was stirred at room temperature for 1 hour. The reaction mixture was diluted with EA (50 mL), treated with saturated NaHCO₃ aq. to pH-8, washed with brine (20 mL×3), dried over Na₂SO₄ and concentrated to dryness. The resulting residue was purified by flash column chromatography to give the titled compound (240 mg, 87%).

### Step D: 1-(2-(1-(difluoromethoxy)ethyl)-4-fluorophenyl)ethan-1-ol

To a solution of 1-(2-(1-(difluoromethoxy)ethyl)-4-fluorophenyl)ethan-1-one (240 mg, 1.0 mmol) in MeOH (5 mL) was added NaBH₄ (57 mg, 1.5 mmol) at room temperature and the resulting mixture was stirred at room temperature for 2 hours. The reaction mixture was concentrated to dryness. The resulting residue was treated with saturated NaHCO₃ aq·, extracted with EA (20 mL×3). The combined organic layers were washed with brine (20 mL×3), dried over Na₂SO₄ and concentrated to dryness. The resulting residue was purified by flash column chromatography to give the titled compound (200 mg, 83%).

### Step E: 2-(7-((2S,5R)-4-(1-(2-(1-(difluoromethoxy)ethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile

To a solution of 1-(2-(1-(difluoromethoxy)ethyl)-4-fluorophenyl)ethan-1-ol (60 mg, 0.3 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (65 mg, 0.2 mmol) and (cyanomethyl)trimethyl phosphonium iodide (97 mg, 0.4 mmol) in CH₃CN (2 mL) was added DIPEA (103 mg, 0.8 mmol). The mixture was sealed in a bottle and heated at 100° C. for 16 hours. Then the mixture was cooled to room temperature, diluted with water, extracted with EA (50 mL×2), washed with brine, dried over Na₂SO₄, filtered, and concentrated to dryness. The resulting residue was purified by flash column chromatography (DCM:MeOH=15:1) and Prep-HPLC (Method A) to give the titled compound (4.3 mg, 3%). 1HNMR (400 MHz, DMSO-d₆) δ 8.05 (s, 1H), 7.75-7.63 (m, 1H), 7.27-7.21 (m, 2H), 6.97-6.59 (m, 1H), 5.68 (s, 2H), 5.48-5.45 (m, 1H), 3.95-3.88 (m, 1H), 3.56-3.54 (m, 1H), 3.34 (s, 3H), 2.91-2.80 (m, 1H), 2.09-1.95 (m, 1H), 1.55-1.54 (m, 3H), 1.38-1.31 (m, 6H), 1.17-1.11 (m, 3H), 0.99-0.92 (m, 2H) ppm. MS: M/e 503 (M+1)⁺.

### Compound A329: 2-(7-((2S,5R)-4-(1-(4-fluoro-2-(1-methoxycyclopropyl)phenyl)ethyl)-2,5-dimethylpiperazin-1-yl)-4-methyl-5-oxo-4,5-dihydro-2H-pyrazolo[4,3-b]pyridin-2-yl)acetonitrile

### Step A: ((1-(2-bromo-5-fluorophenyl)vinyl)oxy)(tert-butyl)dimethylsilane

To a solution of 1-(2-bromo-5-fluorophenyl)ethan-1-one (4.0 g, 18.43 mmol) and Et₃N (7.45 g, 73.72 mmol) in DCM (100 mL) was added TBSOTf (9.74 g, 36.86 mmol). The reaction was stirred at room temperature overnight. The reaction mixture was diluted with water, extracted with DCM (100 mL×2), washed with brine, dried over Na₂SO₄, filtered, and concentrated to dryness. The resulting residue was purified by flash column chromatography (PE:EA=20:1) to give the titled compound (4 g, 64%). ¹H NMR (400 MHz, DMSO-d₆) δ 7.67 (dd, J=8.8, 5.4 Hz, 1H), 7.24 (dd, J=9.3, 3.1 Hz, 1H), 7.17 (d, J=3.2 Hz, 1H), 4.65 (d, J=1.5 Hz, 1H), 4.57 (d, J=1.5 Hz, 1H), 0.88 (s, 9H), 0.13 (s, 6H).

### Step B: (1-(2-bromo-5-fluorophenyl)cyclopropoxy)(tert-butyl)dimethylsilane

To a solution of diethylzinc (2 ml, 4.0 mmol, 2M) in DCM (30 mL) at 0° C. was degassed 3 times under N₂ atmosphere. Then added TFA (456 mg, 4 mmol) very slowly. 10 mins later, added I₂CH₂ (1072 mg, 4.0 mmol). 5 mins later, added ((1-(2-bromo-5-fluorophenyl)vinyl)oxy)(tert-butyl)dimethylsilane (660 mg, 2.0 mmol). The mixture solution was stirred at room temperature for 12 hours. The reaction mixture was diluted with water, extracted with DCM (100 mL×2), washed with brine, dried over Na₂SO₄, filtered, and concentrated to dryness. The resulting residue was purified by flash column chromatography (PE:EA=20:1) to give the titled compound (500 mg, 73%). ¹H NMR (400 MHz, CDCl₃) δ 7.52 (dd, J=8.7, 5.4 Hz, 1H), 7.06 (dd, J=9.1, 3.1 Hz, 1H), 6.86 (ddd, J=11.2, 8.3, 3.0 Hz, 1H), 1.13 (dd, J=7.4, 5.4 Hz, 2H), 0.92 (dd, J=7.3, 5.5 Hz, 2H), 0.78 (s, 9H), 0.12 (d, J=2.9 Hz, 6H).

### Step C: 1-(2-bromo-5-fluorophenyl)cyclopropan-1-ol

To a solution of (1-(2-bromo-5-fluorophenyl)cyclopropoxy)(tert-butyl)dimethylsilane (500 mg, 1.453 mmol) in THF (30 mL) was added TBAF (4.3 mL, 1M, 4.36 mmol). The mixture was stirred at room temperature for 4 hours. The reaction mixture was quenched with water and extracted with EA (45 mL×2). The combined organic layers were washed with brine, dried over Na₂SO₄, filtered and concentrated to dryness. The resulting residue was purified by flash column chromatography (PE:EA=10:1) to give the titled compound (310 mg, 93%). ¹H NMR (400 MHz, CDCl₃) δ 7.53 (dd, J=8.7, 5.2 Hz, 1H), 7.13 (dd, J=9.0, 3.0 Hz, 1H), 6.91 (td, J=8.3, 3.1 Hz, 1H), 1.28-1.24 (m, 2H), 0.98 (dd, J=7.2, 5.7 Hz, 2H), 0.91 (s, 1H).

### Step D: 1-bromo-4-fluoro-2-(1-methoxycyclopropyl)benzene

To a solution of 1-(2-bromo-5-fluorophenyl)cyclopropan-1-ol (300 mg, 1.304 mmol) in THF (30 mL) at 0° C. was added NaH (156 mg, 60%, 3.912 mmol). Then added CH₃I (370 mg, 2.608 mmol). The mixture was stirred at room temperature for 1 hours. The reaction mixture was quenched with water and extracted with EA (30 mL×2). The combined organic layers were washed with brine, dried over Na₂SO₄, filtered and concentrated to dryness. The resulting residue was purified by flash column chromatography (PE:EA=10:1) to give the titled compound (200 mg, 63%).

### Step E: 1-(4-fluoro-2-(1-methoxycyclopropyl)phenyl)ethan-1-one

To a solution of 1-bromo-4-fluoro-2-(1-methoxycyclopropyl)benzene, tributyl(1-ethoxyvinyl)stannane (592 mg, 1.639 mmol), Pd(PPh₃)₂Cl₂ (115 mg, 0.164 mmol) in toluene (20 mL) was stirred at 90° C. under N₂ for 4 hours. The reaction mixture was quenched with saturated NaHCO₃ aq·(20 mL), extracted with EA (30 mL×2), combined, washed brine (20 mL×2), dried and concentrated to dryness. The resulting oil was diluted with THF (20 mL). Then to the solution was added HCl (4 mL, 4 M in 1,4-dioxane) in drops and the mixture was stirred at room temperature for 1 hour. The reaction mixture was diluted with EA (30 mL), treated with saturated NaHCO₃ aq. to pH-8, washed with brine (20 mL×3), dried over Na₂SO₄ and concentrated to dryness. The resulting residue was purified by flash column chromatography (PE:EA=10:1) to give the titled compound (150 mg, 88%). ¹H NMR (400 MHz, CDCl₃) δ 7.37 (dd, J=8.4, 5.8 Hz, 1H), 7.01 (td, J=8.2, 2.5 Hz, 1H), 6.83 (dd, J=9.8, 2.5 Hz, 1H), 3.07 (s, 3H), 2.61 (s, 3H), 1.25-1.19 (m, 2H), 1.10-1.05 (m, 2H).

### Step F: 1-(4-fluoro-2-(1-methoxycyclopropyl)phenyl)ethan-1-ol

To a solution of 1-(4-fluoro-2-(1-methoxycyclopropyl)phenyl)ethan-1-one (150 mg, 0.721 mmol) in CH₃OH (10 mL) was added NaBH₄ (110 mg, 2.885 mmol). The reaction was stirred at room temperature for 1 hours. The reaction mixture was quenched with saturated NH₄Cl, extracted with EA (30 mL×2), washed with brine, dried over Na₂SO₄, filtered, and concentrated to dryness. The resulting residue was purified by flash column chromatography (PE:EA=5:1) to give the titled compound (120 mg, 79%).

### Step G: 2-(7-((2S,5R)-4-(1-(4-fluoro-2-(1-methoxycyclopropyl)phenyl)ethyl)-2,5-dimethylpiperazin-1-yl)-4-methyl-5-oxo-4,5-dihydro-2H-pyrazolo[4,3-b]pyridin-2-yl)acetonitrile

A solution of Intermediate 10 (86 mg, 0.286 mol), 1-(4-fluoro-2-(1-methoxycyclopropyl)phenyl)ethan-1-ol (120 mg, 0.571 mol), (cyanomethyl)trimethylphosphonium iodide (139 mg. 0.571 mol) and DIPEA (111 mg, 0.858 mol) in CH₃CN (2 ml). The mixture solution was degassed 3 times under N₂ atmosphere. Then the mixture solution was stirred at 105° C. for 24 hours. The reaction mixture was concentrated under reduced pressure. The resulting residue was purified by Prep-TLC (DCM:MeOH=15:1) and Prep-HPLC (Method A) to give the titled compound (38 mg, 27%). ¹H NMR (400 MHz, DMSO-d₆) δ 7.99 (d, J=2.5 Hz, 1H), 7.84-7.66 (m, 1H), 7.19 (d, J=9.5 Hz, 1H), 7.08 (t, J=8.5 Hz, 1H), 5.62 (d, J=5.8 Hz, 2H), 5.40 (d, J=9.0 Hz, 1H), 4.42-4.26 (m, 1H), 3.60-3.43 (m, 1H), 3.28 (s, 3H), 3.25-3.04 (m, 2H), 3.00 (d, J=5.4 Hz, 3H), 2.93-2.75 (m, 2H), 2.72-2.56 (m, 1H), 1.31 (d, J=6.7 Hz, 1H), 1.22 (d, J=6.3 Hz, 3H), 1.18-1.12 (m, 2H), 1.11-1.03 (m, 4H), 0.99 (d, J=6.3 Hz, 3H) ppm. MS: M/e 493 (M+1)⁺

### Compound A331: 2-(7-((2S,5R)-4-(1-(4,4-difluorochroman-6-yl)ethyl)-2,5-dimethylpiperazin-1-yl)-4-methyl-5-oxo-4,5-dihydro-2H-pyrazolo[4,3-b]pyridin-2-yl)acetonitrile

### Step A: 6-bromospiro[chromane-4,2′-[1,3]dithiolane]

To a stirred solution of 6-bromo-chroman-4-one (2.0 g, 8.8 mmol) in DCM (40 mL) at 0° C. are added 1,2-ethanedithiol (1.6 g, 17.6 mmol) and boron trifluoride etherate (640 mg, 4.4 mmol). The reaction mixture is stirred at room temperature for 16 hours before it is poured into 1.0 N NaOH solution. The mixture is extracted with DCM and the organic layers are separated, concentrated and purified by silica chromatography (10% EtOAc in PE) to give the titled compound (2.3 g, 86%).

### Step B: 6-bromo-4,4-difluorochromane

A suspension of N-iodosuccinimide (3.4 g, 15 mmol) in DCM (50 mL) is cooled down to −70° C. Hydrogen fluoride pyridine complex (4.2 g, 30 mmol, 70%) is added dropwise. A solution of 6-bromospiro[chromane-4,2′-[1,3]dithiolane] (2.3 g, 7.6 mmol) in DCM (cooled at −70° C.) is added dropwise and the mixture is stirred at −70° C. for 30 min. Then the reaction solution is poured into a mixture of hexane and DCM and the resulting mixture is filtered. The filtrates were washed with Sodium bisulfite aqueous. The organic phases were concentrated and purified by silica chromatography (10% EtOAc in PE) to give the titled compound (1.3 g, 68%).

### Step C: 1-(4,4-difluorochroman-6-yl)ethan-1-one

A mixture of 6-bromo-4,4-difluorochromane (500 mg, 2 mmol), tributyl(1-ethoxyvinyl)stannane (1.4 g, 4 mmol) and Pd(PPh₃)₂Cl₂ (140 mg, 0.2 mmol) in toluene (10 mL) was stirred at 100° C. under N₂ for 16 hrs.

The mixture was cooled and diluted with EA (50 mL), washed with brine (20 mL×2). The mixture was filtered through a celite pad and the organic layer was treated with HCl/Dioxane (4M, 5 mL), and washed with brine (20 mL×2), NaHCO₃ (20 mL×2), dried over Na₂SO₄, filtered and concentrated to dryness. The resulting residue was purified by flash column chromatography to give the titled compound (320 mg, 75%).

### Step D: 1-(4,4-difluorochroman-6-yl)ethan-1-ol

To a solution of 5-acetyl-2-methylisoindoline-1,3-dione (320 mg, 1.5 mmol) in MeOH (10 mL) was added NaBH₄ (80 mg, 2.1 mmol) at room temperature and the mixture was stirred at RT for 2 hours. The mixture was treated with NaHCO₃ (50 mL), extracted with EA (50 mL×2). The combined extracts was washed with brine (50 mL×2), dried over Na₂SO₄ and concentrated to dryness and purified by flash column chromatography to give the title compound (280 mg, 86%).

### Step E: 2-(7-((2S,5R)-4-(1-(4,4-difluorochroman-6-yl)ethyl)-2,5-dimethylpiperazin-1-yl)-4-methyl-5-oxo-4,5-dihydro-2H-pyrazolo[4,3-b]pyridin-2-yl)acetonitrile

To a solution of 1-(4,4-difluorochroman-6-yl)ethan-1-ol (60 mg, 0.28 mmol), 2-(7-((2S,5R)-2,5-dimethylpiperazin-1-yl)-4-methyl-5-oxo-4,5-dihydro-2H-pyrazolo[4,3-b]pyridin-2-yl)acetonitrile (71 mg, 0.18 mmol) and (cyanomethyl)trimethyl phosphonium iodide (68 mg, 0.28 mmol) in CH₃CN (5 mL) was added DIPEA (116 mg, 0.9 mmol). The mixture was sealed in a bottle and heated at 100° C. for 16 hours. The mixture was cooled to room temperature, diluted with water, extracted with EA (10 mL×2), washed with brine, dried over Na₂SO₄, filtered, and concentrated to dryness. The resulting residue was purified by Prep-TLC(DCM:MeOH=15:1) and Prep-HPLC (Method A) to give the titled compound (0.88 mg, 1%). ¹H NMR (400 MHz, CDCl₃) δ 8.80-8.50 (m, 1H), 7.59-7.43 (m, 2H), 7.05-7.00 (m, 1H), 5.75-5.65 (m, 1H), 5.21-5.15 (m, 2H), 4.50-4.25 (m, 4H), 4.01-3.95 (m, 1H), 3.55-3.25 (s, 5H), 3.07-2.95 (m, 1H), 2.75-2.62 (m, 1H), 2.52-2.48 (m, 2H), 2.02-1.88 (m, 3H), 1.57-1.55 (m, 3H), 1.30-1.18 (m, 3H) ppm. MS: M/e 497 (M+1)⁺.

### Compound A332: 2-(7-((2S,5R)-5-ethyl-4-((4-fluorophenyl)(5-(trifluoromethyl)pyridin-2-yl)methyl)-2-methylpiperazin-1-yl)-4-methyl-5-oxo-4,5-dihydro-2H-pyrazolo[4,3-b]pyridin-2-yl)acetonitrile

### Step A: tert-butyl (2S,5R)-5-ethyl-4-((4-fluorophenyl)(5-(trifluoromethyl)pyridin-2-yl)methyl)-2-methylpiperazine-1-carboxylate

A mixture of tert-butyl (2S,5R)-5-ethyl-2-methylpiperazine-1-carboxylate (912 mg, 4 mmol), (4-fluorophenyl)(5-(trifluoromethyl)pyridin-2-yl)methanol (1.3 g, 4.8 mmol), (cyanomethyl)trimethylphosphonium iodide (1.9 g, 8 mmol) and DIPEA (2 g, 16 mmol) in CH₃CN (15 mL) was heated to 100° C. overnight under N₂ atmosphere. The mixture was cooled to room temperature, diluted with water, extracted with EtOAc. The combined organic layers were washed with brine, dried over Na₂SO₄ and concentrated to dryness. The resulting residue was purified by flash column chromatography (EtOAc/PE=1/3) to give the titled compound (700 mg, 36%). MS: M/e 482 (M+1)⁺.

### Step B: (2R,5S)-2-ethyl-1-((4-fluorophenyl)(5-(trifluoromethyl)pyridin-2-yl)methyl)-5-methylpiperazine

To a solution of tert-butyl (2S,5R)-5-ethyl-4-((4-fluorophenyl)(5-(trifluoromethyl)pyridin-2-yl)methyl)-2-methylpiperazine-1-carboxylate (700 mg, 1.5 mmol) in DCM (10 mL) were added TFA (3 mL) at room temperature. The resulting mixture was stirred at room temperature for 3 hours. The reaction solvent was removed under vacuum. The resulting residue (1.4 g, crude) was used in the next step directly without further purification. MS: M/e 382 (M+1)⁺.

### Step C: 7-((2S,5R)-5-ethyl-4-((4-fluorophenyl)(5-(trifluoromethyl)pyridin-2-yl)methyl)-2-methylpiperazin-1-yl)-4-methyl-2-(tetrahydro-2H-pyran-2-yl)-2,4-dihydro-5H-pyrazolo[4,3-b]pyridin-5-one

A mixture of Intermediate 2 (76 mg, 0.2 mmol), (2R,5S)-2-ethyl-1-((4-fluorophenyl)(5-(trifluoromethyl)pyridin-2-yl)methyl)-5-methylpiperazine (106 mg, 0.28 mmol) and DIPEA (129 mg, 1 mmol) in CH₃CN (4 ml) was heated to 90° C. overnight under N₂ atmosphere. The solvent was removed under vacuum. The resulting residue was purified by Prep-TLC (DCM/MeOH=13/1) to give the titled compound (40 mg, 33%). MS: M/e 613 (M+1)⁺.

### Step D: 7-((2S,5R)-5-ethyl-4-((4-fluorophenyl)(5-(trifluoromethyl)pyridin-2-yl)methyl)-2-methylpiperazin-1-yl)-4-methyl-2,4-dihydro-5H-pyrazolo[4,3-b]pyridin-5-one

To a solution of 7-((2S,5R)-5-ethyl-4-((4-fluorophenyl)(5-(trifluoromethyl)pyridin-2-yl)methyl)-2-methylpiperazin-1-yl)-4-methyl-2-(tetrahydro-2H-pyran-2-yl)-2,4-dihydro-5H-pyrazolo[4,3-b]pyridin-5-one (40 mg, 0.065 mmol) in MeOH (2 mL) was added HCl (g) in dioxane solution (2 mL, 8 mmol, 4M) at room temperature. The resulting mixture was stirred at room temperature for 2 hours. The reaction solvent was removed under vacuum to give the titled compound (40 mg, crude), which was used in the next step directly without further purification. MS: M/e 529 (M+1)⁺.

### Step E: 2-(7-((2S,5R)-5-ethyl-4-((4-fluorophenyl)(5-(trifluoromethyl)pyridin-2-yl)methyl)-2-methylpiperazin-1-yl)-4-methyl-5-oxo-4,5-dihydro-2H-pyrazolo[4,3-b]pyridin-2-yl)acetonitrile

To a solution of 7-((2S,5R)-5-ethyl-4-((4-fluorophenyl)(5-(trifluoromethyl)pyridin-2-yl)methyl)-2-methylpiperazin-1-yl)-4-methyl-2,4-dihydro-5H-pyrazolo[4,3-b]pyridin-5-one (30 mg, 0.056 mmol) and K₂CO₃ (24 mg, 0.17 mmol) in DMF (2 mL) were added 2-iodoacetonitrile (19 mg, 0.11 mmol). The resulting mixture was stirred at room temperature overnight. The reaction mixture was diluted with EtOAc (20 mL) and washed with brine (20 mL×3). The organic layers were concentrated under reduced pressure. The resulting residue was purified by Prep-TLC (DCM/MeOH=13/1) to give the titled compound (12 mg, 37%). ¹H NMR (400 MHz, CD₃OD) δ 8.79-8.72 (m, 1H), 8.16-7.96 (m, 2H), 7.92 (s, 1H), 7.66-7.53 (m, 2H), 7.07 (q, J=8.9 Hz, 2H), 5.60 (s, 1H), 5.44 (s, 2H), 5.03-4.94 (m, 1H), 3.58-3.43 (m, 1H), 3.43 (s, 3H), 3.13-2.99 (m, 1H), 2.88 (d, J=8.5 Hz, 2H), 2.58 (s, 1H), 2.49-2.34 (m, 1H), 1.73-1.54 (m, 2H), 1.44 (dd, J=9.8, 6.6 Hz, 3H), 0.70 (dd, J=16.7, 7.5 Hz, 3H). MS: M/e 568 (M+1)⁺.

### Compound A335: 2-(but-2-yn-1-yl)-7-((2S,5R)-4-(1-(3-(difluoromethyl)quinoxalin-6-yl)ethyl)-2,5-dimethylpiperazin-1-yl)-4-methyl-2,4-dihydro-5H-pyrazolo[4,3-b]pyridin-5-one

### Step A: tert-butyl (2R,5S)-4-(2-(but-2-yn-1-yl)-4-methyl-5-oxo-4,5-dihydro-2H-pyrazolo[4,3-b]pyridin-7-yl)-2,5-dimethylpiperazine-1-carboxylate

To a stirred solution of the compound of Step C for Intermediate 10 (361 mg, 1 mmol) in DMF/H₂O (5 mL/2 mL) was added K₂CO₃ (276 mg, 2 mmol), followed by 1-bromobut-2-yne (266 mg, 2 mmol). After the addition, the reaction mixture was stirred overnight. The reaction mixture was treated with H₂O (50 mL) and extracted with EtOAc (15 mL×2). The combined organic layers were washed with brine, dried over Na₂SO₄, concentrated and purified by flash column chromatography to give the titled compound (192 mg, 46.5%). MS: M/e 414 (M+1)⁺.

### Step B: 2-(but-2-yn-1-yl)-7-((2S,5R)-2,5-dimethylpiperazin-1-yl)-4-methyl-2,4-dihydro-5H-pyrazolo[4,3-b]pyridin-5-one

To a stirred solution of tert-butyl (2R,5S)-4-(2-(but-2-yn-1-yl)-4-methyl-5-oxo-4,5-dihydro-2H-pyrazolo[4,3-b]pyridin-7-yl)-2,5-dimethylpiperazine-1-carboxylate (192 mg, 0.46 mmol) in CH₂Cl₂ (5 mL) was added TFA (2 mL). After then, the reaction was stirred for 2 hours. The reaction mixture was concentrated, basified to pH=9-10 with aq·K₂CO₃, extracted with CH₂Cl₂/IPA (3/1, 30 mL×2). The combined organic layers were washed with brine, dried over Na₂SO₄, concentrated to give the titled compound (135 mg, 93%). MS: M/e 314 (M+1)⁺.

### Step C: 2-(but-2-yn-1-yl)-7-((2S,5R)-4-(1-(3-(difluoromethyl)quinoxalin-6-yl)ethyl)-2,5-dimethylpiperazin-1-yl)-4-methyl-2,4-dihydro-5H-pyrazolo[4,3-b]pyridin-5-one

A mixture of 2-(but-2-yn-1-yl)-7-((2S,5R)-2,5-dimethylpiperazin-1-yl)-4-methyl-2,4-dihydro-5H-pyrazolo[4,3-b]pyridin-5-one (104.8 mg, 0.335 mmol), 1-(3-(difluoromethyl)quinoxalin-6-yl)ethan-1-ol (75 mg, 0.335 mmol), (cyanomethyl)trimethylphosphonium iodide (243 mg, 1 mmol) and DIPEA (432 mg, 3.35 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Prep-HPLC (Method A) to give the titled compound (58 mg). ¹H NMR (400 MHz, CD₃OD) δ 9.13 (d, J=4.8 Hz, 1H), 8.21-8.09 (m, 3H), 7.90 (s, 1H), 7.15-6.83 (m, 1H), 5.53 (s, 1H), 5.03 (s, 2H), 4.77-4.18 (m, 2H), 4.04-3.80 (m, 1H), 3.72-3.61 (m, 1H), 3.44 (s, 3H), 3.11-2.81 (m, 2H), 2.18 (d, J=12.0 Hz, 0.5H), 1.85 (s, 3H), 1.50-1.40 (m, 4.5H), 1.20 (t, J=6.4 Hz, 3H), 1.05 (d, J=6.4 Hz, 2H) ppm. MS: M/e 520 (M+1)⁺.

### Compound A337: 2-(7-((2S,5R)-4-(1-(2,3-dihydrofuro[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile

### Step A: 2-(2-chloropyridin-3-yl)ethan-1-ol

To a solution of methyl 2-(2-chloropyridin-3-yl)acetate (500 mg, 2.5 mmol) in THF (10 mL) was slowly added a solution of LiAlH₄ (1M, 3.7 mL, 3.7 mmol). The solution was stirred at room temperature for 2 hours. The mixture was poured into water, extracted with EtOAc, washed with water, dried over Na₂SO₄ and concentrated to dryness. The resulting residue was purified by flash column chromatography to give the titled compound (330 mg, 83%). MS: M/e 158 (M+1)⁺.

### Step B: 2,3-dihydrofuro[2,3-b]pyridine

A mixture of 2-(2-chloropyridin-3-yl)ethan-1-ol (300 mg, 1.9 mmol) in toluene (15 mL) was added NaH (60%, 0.15 g, 3.8 mol). The reaction mixture was stirred at 50° C. for 18 hours. The brown residue was dissolved in EtOAc, washed with water, dried over Na₂SO₄ and concentrated to dryness. The resulting residue was purified by flash column chromatography to give the titled compound (190 mg, 82%). MS: M/e 122 (M+1)⁺.

### Step C: 2,3-dihydrofuro[2,3-b]pyridine 7-oxide

To a solution of 2,3-dihydrofuro[2,3-b]pyridine (0.19 g, 1.57 mmol) in DCM (20 mL) was added 3-chlorobenzoperoxoic acid (540 mg, 3.1 mmol). The mixture solution was stirred at room temperature for 2 days. The reaction was concentrated to dryness. The resulting residue was purified by flash column chromatography to give the titled compound (0.14 g, 60%). MS: M/e 138 (M+1)⁺.

### Step D: 2,3-dihydrofuro[2,3-b]pyridine-6-carbonitrile

To a solution of 2,3-dihydrofuro[2,3-b]pyridine 7-oxide (0.12 g, 0.87 mmol) in CH₃CN (5 mL) were added TMSCN (434 mg, 4.4 mmol), Et₃N (354 mg, 3.5 mol). The mixture was stirred at 90° C. overnight. The reaction was concentrated to dryness. The resulting residue was purified by flash column chromatography to give the titled compound (80 mg, 62%). MS: M/e 147 (M+1)⁺.

### Step E: 1-(2,3-dihydrofuro[2,3-b]pyridin-6-yl)ethan-1-one

A mixture of 2,3-dihydrofuro[2,3-b]pyridine-6-carbonitrile (80 mg, 0.54 mmol) in THF (3 mL) at 0° C. was added methyl magnesium bromide (0.55 mL, 3M, 1.54 mol). The reaction mixture was stirred at room temperature for 30 mins. The reaction was quenched by adding saturated solution of NH₄Cl. The resulting mixture was extracted with EtOAc, and then concentrated by using a rotary evaporator, to give the titled compound (80 mg). MS: M/e 164 (M+1)⁺.

### Step F: 1-(2,3-dihydrofuro[2,3-b]pyridin-6-yl)ethan-1-ol

To a solution of 1-(2,3-dihydrofuro[2,3-b]pyridin-6-yl)ethan-1-one (80 mg, 0.49 mmol) in MeOH (5 mL) was added NaBH₄ (19 mg, 0.49 mmol) at room temperature and the resulting mixture was stirred at room temperature for 5 min. The reaction mixture was diluted with DCM and washed with water, dried over Na₂SO₄ and concentrated to give the titled compound (70 mg). MS: M/e 166 (M+1)⁺.

### Step G: 2-(7-((2S,5R)-4-(1-(2,3-dihydrofuro[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile

To a solution of Intermediate 5 (50 mg, 0.15 mmol) in CH₃CN (2 mL) and was added 1-(2,3-dihydrofuro[2,3-b]pyridin-6-yl)ethan-1-ol (50 mg, 0.3 mmol), (cyanomethyl)trimethylphosphonium iodide (110 mg, 0.45 mmol) and DIPEA (197 mg, 1.5 mmol). The resulting mixture was stirred at 105° C. overnight.

The reaction solvent was removed under reduce pressure, The reaction was diluted with DCM and washed with water. The organic layer was separated, dried by Na₂SO₄, filtered, and concentrated to dryness. The resulting residue was purified by flash column chromatography (DCM/MeOH) to give the titled compound (16 mg). ¹H NMR (400 MHz, CD₃OD) δ 7.92 (d, J=2.5 Hz, 1H), 7.64-7.59 (m, 1H), 7.04 (dd, J=22.9, 7.4 Hz, 1H), 5.55 (s, 1H), 5.47 (d, J=2.9 Hz, 2H), 4.64 (td, J=8.7, 3.5 Hz, 3H), 3.79-3.49 (m, 2H), 3.43 (s, 3H), 3.29-3.21 (m, 3H), 3.16-2.29 (m, 3H), 2.15-1.48 (m, 4H), 1.31 (dd, J=12.8, 6.6 Hz, 3H), 0.97 (dt, J=24.1, 7.4 Hz, 3H), 0.78-0.65 (m, 3H) ppm. MS: M/e 476 (M+1)⁺.

### Compound A339: 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)propyl)phenyl)-2-methylpropanenitrile

### Step A: 2-(4-(1-hydroxyprolyl)phenyl)-2-methylpropanenitrile

2-(4-bromophenyl)-2-methylpropanenitrile (448 mg, 2 mmol) dissolved in tetrahydrofuran (5 mL) and cooled to −78° C. A solution of n-butyllithium in hexane (1.6 M, 1.9 mL, 3 mmol) added drop wise to cold reaction mixture and stirred at −78° C. for 1 hour. Propionaldehyde (174 mg, 3 mmol) was added drop wise at −78° C. Once addition was complete, reaction was warmed up to 0° C. for 1 hour. Saturated aqueous ammonium chloride and water added. The reaction mixture was extracted with EtOAc and organic layers was washed with brine, dried over magnesium sulfate, filtered and concentrated. The resulting residue was purified by flash column chromatography to give the titled compound (270 mg, 66%). MS: M/e 204 (M+1)⁺.

### Step B: 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)propyl)phenyl)-2-methylpropanenitrile

A mixture of 2-(4-(1-hydroxypropyl)phenyl)-2-methylpropanenitrile (30 mg, 0.15 mmol), Intermediate 5 (50 mg, 0.15 mmol), (cyanomethyl)trimethylphosphonium iodide (72 mg, 0.3 mmol) and DIPEA (77 mg, 0.6 mmol) in MeCN (3 mL) was stirred at 100° C. overnight. The reaction mixture was diluted with EtOAc, washed with brine, dried over Na₂SO₄ and concentrated to dryness. The resulting residue was purified by Prep-TLC to give the titled compound (8 mg, 10%). ¹H NMR (400 MHz, CD₃OD) δ 7.92 (s, 1H), 7.57-7.47 (m, 2H), 7.41 (t, J=8.5 Hz, 2H), 5.54 (s, 1H), 5.47 (s, 2H), 3.65-3.40 (m, 5H), 3.19 (s, 1H), 2.99 (s, 1H), 2.73-2.30 (m, 2H), 1.94 (s, 2H), 1.86-1.60 (m, 9H), 1.49 (s, 2H), 1.06-0.94 (m, 3H), 0.76-0.55 (m, 6H) ppm. MS: M/e 514 (M+1)⁺.

### Compound A341: 2-(7-((2S,5R)-4-(1-(4-((2,2-dimethylmorpholino)methyl)phenyl)ethyl)-2,5-dimethylpiperazin-1-yl)-4-methyl-5-oxo-4,5-dihydro-2H-pyrazolo[4,3-b]pyridin-2-yl)acetonitrile

### Step A: 4-(4-bromobenzyl)-2,2-dimethylmorpholine

A mixture of 1-bromo-4-(bromomethyl)benzene (500 mg, 2 mmol), 2,2-dimethylmorpholine (253 mg, 2.2 mmol) and DIPEA (774 mg, 3 mmol) in acetonitrile (10 ml) was stirred at 50° C. for 2 hours. The reaction mixture was concentrated to dryness. The resulting residue was purified by flash column chromatography to give the titled compound (610 mg, crude). MS: M/e 284 (M+1)⁺.

### Step B: 1-(4-((2,2-dimethylmorpholino)methyl)phenyl)ethan-1-one

A mixture of 4-(4-bromobenzyl)-2,2-dimethylmorpholine (600 mg, 2.12 mmol), tributyl(1-ethoxyvinyl)stannane (1.15 g, 3.2 mmol) and Pd(PPh₃)₂Cl₂ (147 mg, 0.21 mmol) in toluene (5 mL) was stirred at 100° C. under N₂ overnight. To the resulting solution was added TFA (1 mL) in drops and the mixture was stirred at room temperature for 30 minutes. The mixture was diluted with EtOAc (20 mL), treated with saturated Na₂CO₃ aq. to pH ˜8, washed with brine (20 mL×3), dried and concentrated to dryness. The resulting residue was purified by flash column chromatography to give the titled compound (70 mg, 13%). MS: M/e 248 (M+1)⁺.

### Step C: 1-(4-((2,2-dimethylmorpholino)methyl)phenyl)ethan-1-ol

To a solution of 1-(4-((2,2-dimethylmorpholino)methyl)phenyl)ethan-1-one (70 mg, 0.28 mmol) in MeOH (3 mL) was added NaBH₄ (10 mg, 0.28 mmol) at 0° C. and the mixture was stirred at room temperature for 30 minutes. The mixture was treated with water, extracted with DCM. The combined organic layers were washed with brine, dried over Na₂SO₄ and concentrated to dryness. The resulting residue (70 mg, crude) was used in the next step directly without further purification. MS: M/e 250 (M+1)⁺.

### Step D: 2-(7-((2S,5R)-4-(1-(4-((2,2-dimethylmorpholino)methyl)phenyl)ethyl)-2,5-dimethylpiperazin-1-yl)-4-methyl-5-oxo-4,5-dihydro-2H-pyrazolo[4,3-b]pyridin-2-yl)acetonitrile

A mixture of 1-(4-((2,2-dimethylmorpholino)methyl)phenyl)ethan-1-ol (70 mg, 0.28 mmol), Intermediate 10 (76 mg, 0.25 mmol), (cyanomethyl)trimethylphosphonium iodide (121 mg, 0.5 mmol) and DIPEA (129 mg, 1 mmol) in MeCN (2 mL) was stirred at 100° C. overnight. The reaction mixture was diluted with EtOAc, washed with brine, dried over Na₂SO₄ and concentrated to dryness. The resulting residue was purified by Prep-HPLC (Method A) to give the titled compound (2 mg, 2%, FA salt). ¹H NMR (400 MHz, CD₃OD) δ 8.23 (s, 1H), 7.94 (s, 1H), 7.48-7.34 (m, 4H), 5.57 (s, 1H), 5.48 (s, 2H), 4.85-4.37 (m, 2H), 3.94-3.77 (m, 2H), 3.69 (d, J=18.9 Hz, 4H), 3.44 (s, 3H), 3.19-3.03 (m, 1H), 2.94 (dd, J=22.1, 11.4 Hz, 1H), 2.57 (s, 2H), 2.49-2.31 (m, 3H), 1.49-1.05 (m, 15H) ppm. MS: M/e 532 (M+1)⁺.

### Compound A343: 2-(7-((2S,5R)-4-(1-(4-(((2S,6R)-2,6-dimethylmorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile

### Step A: methyl 4-(((2S,6R)-2,6-dimethylmorpholino)methyl)benzoate

A solution of methyl 4-formylbenzoate (802 mg, 5 mmol), (2S,6R)-2,6-dimethylmorpholine (575 mg, 5 mmol) and AcOH (0.5 mL) in CH₂Cl₂ (10 mL) was stirred for 2 hours, then NaBH(OAc)₃ (1.59 g, 7.5 mmol) was added. After then, the mixture was stirred overnight. The mixture was diluted with CH₂Cl₂ (20 mL), then washed with H₂O, brine, dried over Na₂SO₄, concentrated and purified by flash column chromatography to give the titled compound (480 mg, 36.5%). MS: M/e 264 (M+1)⁺.

### Step B: 4-(((2S,6R)-2,6-dimethylmorpholino)methyl)benzoic acid

To a stirred solution of methyl 4-(((2S,6R)-2,6-dimethylmorpholino)methyl)benzoate (480 mg, 1.83 mmol) in MeOH (5 mL) was added aq·NaOH (2.0 M, 2 mL). After then, the mixture was stirred for 2 hours. The mixture was concentrated to give the aqueous layer, which was acidified to pH=3-4 with aq·HCl, then extracted with CH₂Cl₂/IPA (3/1, 20 mL×4). The combined organic layers were washed with brine, dried over Na₂SO₄, concentrated to give the titled compound (420 mg, 92.2%). MS: M/e 250 (M+1)⁺.

### Step C: 4-(((2S,6R)-2,6-dimethylmorpholino)methyl)-N-methoxy-N-methylbenzamide

A mixture of 4-(((2S,6R)-2,6-dimethylmorpholino)methyl)benzoic acid (420 mg, 1.62 mmol), N,O-dimethylhydroxylamine hydrochloride (189 mg, 1.94 mmol), HATU (741 mg, 1.94 mmol) and DIEPA (418 mg, 3.24 mmol) in CH₂Cl₂ (10 mL) was a stirred overnight. The reaction mixture was washed with brine, dried over Na₂SO₄, concentrated and purified by flash column chromatography to give the titled compound (130 mg, 22.7%). MS: M/e 293 (M+1)⁺.

### Step D: 1-(4-(((2S,6R)-2,6-dimethylmorpholino)methyl)phenyl)ethan-1-one

To a stirred solution of 4-(((2S,6R)-2,6-dimethylmorpholino)methyl)-N-methoxy-N-methylbenzamide (130 mg, 0.43 mmol) in dry THF (10 mL) was added dropwise MeMgBr (3.0 M, 0.17 mL, 0.52 mmol) at 0° C. After then, the mixture was stirred for half an hour. The reaction was quenched with aq·NH₄Cl, extracted with EtOAc (10 mL×3). The combined organic layers were washed with brine, dried over Na₂SO₄, concentrated and purified by flash column chromatography to give the titled compound (20 mg, 18%). MS: M/e 248 (M+1)⁺.

### Step E: 1-(4-(((2S,6R)-2,6-dimethylmorpholino)methyl)phenyl)ethan-1-ol

To a stirred solution of 1-(4-(((2S,6R)-2,6-dimethylmorpholino)methyl)phenyl)ethan-1-one (20 mg, 0.078 mmol) in MeOH (10 mL) was added NaBH₄ (3 mg, 0.078 mmol). After then, the mixture was stirred for 10 min. The reaction mixture was quenched with aq·NH₄Cl and extracted with CH₂Cl₂/IPA (3/1, 10 mL×3). The combined organic layers were washed with brine, dried over Na₂SO₄, concentrated to give the titled compound (20 mg, 99%). MS: M/e 250 (M+1)⁺.

### Step F: 2-(7-((2S,5R)-4-(1-(4-(((2S,6R)-2,6-dimethylmorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile

A mixture of Intermediate 5 (32.8 mg, 0.1 mmol), 1-(4-(((2S,6R)-2,6-dimethylmorpholino)methyl)phenyl)ethan-1-ol (20 mg, 0.08 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to give the residue, which was purified by Prep-HPLC (Method A) to give the titled compound (3 mg). ¹H NMR (400 MHz, CD₃OD) δ 7.92 (d, J=2.4 Hz, 1H), 7.41-7.25 (m, 4H), 5.54 (d, J=5.2 Hz, 1H), 5.46 (d, J=3.6 Hz, 2H), 3.77-3.55 (m, 3H), 3.52 (s, 2H), 3.50-3.45 (m, 1H), 3.43 (s, 3H), 3.26-2.82 (m, 2H), 2.74 (t, J=10.6 Hz, 2H), 2.65-2.32 (m, 2H), 2.15-1.92 (m, 1H), 1.86-1.47 (m, 6H), 1.38-1.29 (m, 3H), 1.10 (t, J=5.6 Hz, 6H), 1.05-0.91 (m, 3H), 0.62 (dt, J=35.2, 7.2 Hz, 3H) ppm. MS: M/e 560 (M+1)⁺.

### Compound A345: 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-3-fluorophenyl)-2-methylpropanenitrile

### Step A: 2-(4-bromo-3-fluorophenyl)-2-methylpropanenitrile

To a stirred suspension of 60% sodium hydride (360 mg, 9 mmol) in dry dimethylformamide (5 mL) under nitrogen, dropwise, a solution of 2-(4-bromo-3-fluorophenyl)acetonitrile (642 mg, 3 mmol) in dry dimethylformamide (5 mL). The reaction mixture stirred for 30 minutes. A solution of iodomethane (1.06 g, 7.5 mmol) in dry dimethylformamide (1 mL) was added dropwise. The reaction mixture was stirred at room temperature overnight. The reaction mixture was quenched with water and extracted with ethyl acetate (20 ml*3). The combined organic extracts were washed with saturated sodium chloride solution, then dried over magnesium sulfate, filtered and evaporated in vacuo. The resulting residue was purified by flash column chromatography to give the titled compound (330 mg, 46%). MS: M/e 242 (M+1)⁺.

### Step B: 2-(4-acetyl-3-fluorophenyl)-2-methylpropanenitrile

A mixture of 2-(4-bromo-3-fluorophenyl)-2-methylpropanenitrile (120 mg, 0.5 mmol), tributyl(1-ethoxyvinyl)stannane (270 mg, 0.75 mmol) and Pd(PPh₃)₂Cl₂ (35 mg, 0.05 mmol) in toluene (3 mL) was stirred at 100° C. under N₂ overnight. To the resulting solution was added HCl (0.5 mL, con.) in drops and the mixture was stirred at room temperature for 30 minutes. The mixture was diluted with EtOAc (10 mL), treated with saturated NaHCO₃ aq. to pH ˜8, washed with brine (20 mL), dried and concentrated to dryness. The resulting residue was purified by flash column chromatography to give the titled compound (40 mg, 39%). MS: M/e 206 (M+1)⁺.

### Step C: 2-(3-fluoro-4-(1-hydroxyethyl)phenyl)-2-methylpropanenitrile

To a solution of 2-(4-acetyl-3-fluorophenyl)-2-methylpropanenitrile (40 mg, 0.2 mmol) in MeOH (2 mL) was added NaBH₄ (7 mg, 0.16 mmol) at room temperature and the mixture was stirred at room temperature for 30 minutes. The resulting residue was treated with saturated NaHCO₃ aq·(10 mL), extracted with DCM (10 mL×3). The combined organic layers were washed with brine (10 mL), dried over Na₂SO₄ and concentrated to dryness. The resulting residue (40 mg, crude) was used in the next step. MS: M/e 208 (M+1)⁺.

### Step D: 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-3-fluorophenyl)-2-methylpropanenitrile

A mixture of 2-(3-fluoro-4-(1-hydroxyethyl)phenyl)-2-methylpropanenitrile (25 mg, 0.12 mmol), Intermediate 5 (33 mg, 0.1 mmol), (cyanomethyl)trimethylphosphonium iodide (48 mg, 0.2 mmol) and DIPEA (51 mg, 0.4 mmol) in MeCN (1 mL) was stirred at 100° C. overnight. The reaction mixture was diluted with EtOAc (20 mL), washed with brine (10 mL), dried over Na₂SO₄ and concentrated to dryness.

The resulting residue was purified by Prep-HPLC (Method A) to give the titled compound (2 mg, 4%). ¹H NMR (400 MHz, CD₃OD) δ 7.92 (d, J=2.3 Hz, 1H), 7.64 (dt, J=30.7, 7.8 Hz, 1H), 7.37 (t, J=9.0 Hz, 1H), 7.24 (t, J=11.3 Hz, 1H), 5.55 (s, 1H), 5.47 (d, J=3.2 Hz, 2H), 4.22-3.99 (m, 1H), 3.54-3.40 (m, 4H), 3.12 (s, 1H), 2.93 (d, J=4.1 Hz, 1H), 2.69 (d, J=9.2 Hz, 1H), 2.47-2.32 (m, 1H), 2.13-1.90 (m, 1H), 1.73 (d, J=5.8 Hz, 5H), 1.62-1.50 (m, 2H), 1.40-1.25 (m, 6H), 1.06-0.93 (m, 3H), 0.80-0.59 (m, 3H) ppm. MS: M/e 518 (M+1)⁺.

### Compound A347: 2-(7-((2S,5R)-4-(1-(5-(difluoromethyl)pyridin-2-yl)ethyl)-2,5-diethylpiperazin-1-yl)-4-methyl-5-oxo-4,5-dihydro-2H-pyrazolo[4,3-b]pyridin-2-yl)acetonitrile

### Step A: 2-bromo-5-(difluoromethyl)pyridine

To a stirred solution of 6-bromonicotinaldehyde (1.86 g, 10 mmol) in CH₂Cl₂ (40 mL) was added dropwise a solution of DAST (3.2 g, 20 mmol) in CH₂Cl₂ (5 mL) at 0° C. After the addition, the reaction mixture was stirred overnight. The reaction mixture was quenched with aq·NaHCO₃, extracted with CH₂Cl₂ (40 mL×2). The combined organic layers were washed with brine, dried over Na₂SO₄ and concentrated. The resulting residue was purified by flash column chromatography to give the titled compound (1.6 g, 76.9%). MS: M/e 208/210 (M+1)⁺.

### Step B: 1-(5-(difluoromethyl)pyridin-2-yl)ethan-1-one

To a stirred solution of 2-bromo-5-(difluoromethyl)pyridine (1.6 g, 7.7 mmol) in toluene (20 mL) was added tributyl(1-ethoxyvinyl)stannane (2.6 g, 7.2 mmol) and Pd(PPh₃)₂Cl₂ (270 mg, 0.385 mmol). After the addition, the reaction mixture was stirred at 100° C. overnight under N₂. The reaction mixture was cooled to RT and washed with aq·HCl (2.0 M, 5 mL) for 10 minutes. Then extracted with EtOAc (30 mL×2). The combined organic layers were washed with brine, dried over Na₂SO₄ and concentrated to dryness. The resulting residue was purified by flash column chromatography to give the titled compound (400 mg, 30%). MS: M/e 172 (M+1)⁺.

### Step C: 1-(5-(difluoromethyl)pyridin-2-yl)ethan-1-ol

To a stirred solution of 1-(5-(difluoromethyl)pyridin-2-yl)ethan-1-one (400 mg, 2.34 mmol) in MeOH (5 mL) was added NaBH₄ (89 mg, 2.34 mmol). After then, the mixture was stirred for 10 min. The reaction mixture was poured into H₂O (10 mL) and extracted with EtOAc (10 mL×3). The combined organic layers were washed with brine, dried over Na₂SO₄ and concentrated to give the titled compound (80 mg, 20%). MS: M/e 174 (M+1)⁺.

### Step D: 2-(7-((2S,5R)-4-(1-(5-(difluoromethyl)pyridin-2-yl)ethyl)-2,5-diethylpiperazin-1-yl)-4-methyl-5-oxo-4,5-dihydro-2H-pyrazolo[4,3-b]pyridin-2-yl)acetonitrile

A mixture of Intermediate 5 (32.8 mg, 0.1 mmol), 1-(5-(difluoromethyl)pyridin-2-yl)ethan-1-ol (26 mg, 0.15 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄ and concentrated to give the titled Compound A347 (crude), which was further purified by Prep-HPLC (Method A) to give Compound A347a (6 mg) and Compound A347b (8 mg).

Compound A347a (the earlier peak): ¹H NMR (400 MHz, CD₃OD) δ 8.64 (s, 1H), 8.02 (d, J=8.0 Hz, 1H), 7.93 (s, 1H), 7.80 (d, J=8.0 Hz, 1H), 6.91 (t, J=55.6 Hz, 1H), 5.56 (s, 1H), 5.47 (s, 2H), 3.88-3.79 (m, 1H), 3.57-3.51 (m, 1H), 3.43 (s, 3H), 3.32-3.30 (m, 2H), 3.20-3.13 (m, 1H), 2.82-2.75 (m, 1H), 2.27-2.20 (m, 1H), 2.03-1.88 (m, 1H), 1.79-1.63 (m, 2H), 1.60-1.46 (m, 1H), 1.37 (d, J=6.4 Hz, 3H), 1.03 (t, J=6.8 Hz, 3H), 0.65 (t, J=6.8 Hz, 3H) ppm. MS: M/e 484 (M+1)⁺.

Compound A347b (the later peak): ¹H NMR (400 MHz, CD₃OD) δ 8.65 (s, 1H), 8.03 (d, J=8.0 Hz, 1H), 7.92 (s, 1H), 7.75 (d, J=8.0 Hz, 1H), 6.91 (t, J=55.6 Hz, 1H), 5.56 (s, 1H), 5.46 (s, 2H), 4.06-3.98 (m, 1H), 3.43 (s, 3H), 3.41-3.35 (m, 1H), 3.32-3.30 (m, 2H), 3.06-2.91 (m, 2H), 2.36 (s, 1H), 2.17-2.04 (m, 1H), 1.91-1.76 (m, 1H), 1.64-1.53 (m, 2H), 1.39 (d, J=6.4 Hz, 3H), 0.95 (t, J=6.8 Hz, 3H), 0.73 (t, J=6.8 Hz, 3H) ppm. MS: M/e 484 (M+1)⁺

### Compound A348: 2-(7-((2S,5R)-2,5-diethyl-4-(1-(5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile

### Step A: 1-(5-(trifluoromethyl)pyridin-2-yl)ethan-1-ol

To a stirred solution of 5-(trifluoromethyl)picolinaldehyde (525 mg, 3 mmol) in THF (10 mL) was added dropwise MeMgBr (3.0 M, 1 mL, 3 mmol) at 0° C. After stirred for 30 min, the reaction was quenched with aq·NH₄Cl, extracted with EtOAc (30 mL×3). The combined organic layers were washed with brine, dried over Na₂SO₄ and concentrated to dryness. The resulting residue was purified by flash column chromatography to give the titled compound (420 mg, 73.2%). MS: M/e 192 (M+1)⁺.

### Step B: 2-(7-((2S,5R)-2,5-diethyl-4-(1-(5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile

A mixture of Intermediate 5 (32.8 mg, 0.1 mmol), 1-(5-(trifluoromethyl)pyridin-2-yl)ethan-1-ol (29 mg, 0.15 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄ and concentrated to give the titled compound Compound A348 (crude), which was further purified by Prep-HPLC (Method A) to give Compound A348a (8 mg) and Compound A348b (7 mg).

Compound A348a (the earlier peak): ¹H NMR (400 MHz, CD₃OD) δ 8.79 (s, 1H), 8.14 (d, J=8.4 Hz, 1H), 7.93 (s, 1H), 7.88 (d, J=8.4 Hz, 1H), 5.56 (s, 1H), 5.47 (s, 2H), 3.92-3.81 (m, 1H), 3.58-3.51 (m, 1H), 3.44 (s, 3H), 3.32-3.30 (m, 2H), 3.19-3.12 (m, 1H), 2.83-2.76 (m, 1H), 2.26-2.20 (m, 1H), 2.01-1.46 (m, 4H), 1.37 (d, J=6.4 Hz, 3H), 1.03 (t, J=6.8 Hz, 3H), 0.66 (t, J=6.8 Hz, 3H) ppm. MS: M/e 502 (M+1)⁺.

Compound A348b (the later peak): ¹H NMR (400 MHz, CD₃OD) δ 8.80 (s, 1H), 8.14 (d, J=8.0 Hz, 1H), 7.93 (s, 1H), 7.82 (d, J=8.0 Hz, 1H), 5.57 (s, 1H), 5.47 (s, 2H), 4.12-4.02 (m, 1H), 3.43 (s, 3H), 3.39-3.33 (m, 1H), 3.32-3.30 (m, 2H), 3.04-2.93 (m, 2H), 2.37 (s, 1H), 2.16-1.77 (m, 2H), 1.66-1.51 (m, 2H), 1.40 (d, J=6.4 Hz, 3H), 0.95 (t, J=6.8 Hz, 3H), 0.75 (t, J=6.8 Hz, 3H) ppm. MS: M/e 502 (M+1)⁺

### Compound A350: 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-b]pyridin-2-yl)acetonitrile

### Step A: 5-bromo-2-cyclopropylpyridine

To a solution 2,5-dibromopyridine (2.37 g, 10 mmol) and Pd(PPh₃)₄ (1.05 g, 1 mmol) in THF (10 mL) was added cycloropylzinc chloride (0.5M in THF, 22 mL, 11 mmol) and the mixture was stirred under argon atmosphere at 70° C. overnight. Cooled to 23° C., poured into sat. NaHCO₃ solution, extracted with EtOAc, washed with brine, dried over Na₂SO₄. Removal of the solvent in vacuum left a brown oil, which was purified by silica gel chromatography to give the titled compound (1.4 g, crude). MS: M/e 198 (M+1)⁺.

### Step B: 1-(6-cyclopropylpyridin-3-yl)ethan-1-one

A mixture of 5-bromo-2-cyclopropylpyridine (600 mg, 3 mmol), tributyl(1-ethoxyvinyl)stannane (1.6 g, 4.5 mmol) and Pd(PPh₃)₂Cl₂ (210 mg, 0.3 mmol) in toluene (10 mL) was stirred at 100° C. under N₂ overnight. To the resulting solution was added TFA (0.5 mL) in drops and the mixture was stirred at room temperature for 30 minutes. The mixture was diluted with EtOAc (20 mL), treated with saturated NaHCO₃ aq. to pH ˜8, washed with brine (20 mL×3), dried over Na₂SO₄ and concentrated to dryness. The resulting residue was purified by flash column chromatography to give the titled compound (80 mg, 16%). MS: M/e 162 (M+1)⁺.

### Step C: 1-(6-cyclopropylpyridin-3-yl)ethan-1-ol

To a solution of 1-(6-cyclopropylpyridin-3-yl)ethan-1-one (80 mg, 0.5 mmol) in MeOH (2 mL) was added NaBH₄ (15 mg, 0.4 mmol) at room temperature and the mixture was stirred at room temperature for 1 hour. The mixture was treated with water, extracted with DCM. The combined organic layers were washed with brine, dried over Na₂SO₄ and concentrated to dryness. The resulting residue was purified by Prep-TLC to give the titled compound (20 mg, 25%). MS: M/e 164 (M+1)⁺.

### Step D: 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-b]pyridin-2-yl)acetonitrile

A mixture of 1-(6-cyclopropylpyridin-3-yl)ethan-1-ol (30 mg, 0.18 mmol), Intermediate 5 (40 mg, 0.12 mmol), (cyanomethyl)trimethylphosphonium iodide (58 mg, 0.24 mmol) and DIPEA (62 mg, 0.48 mmol) in CH₃CN (3 mL) was stirred at 100° C. overnight. The reaction mixture was diluted with EtOAc, washed with brine, dried over Na₂SO₄ and concentrated to dryness. The resulting residue was purified by Prep-TLC to give the titled compound (1.4 mg, 3%). ¹H NMR (400 MHz, CD₃OD) δ 8.32 (d, J=14.8 Hz, 1H), 7.92 (s, 1H), 7.72 (s, 1H), 7.26-7.14 (m, 1H), 5.55 (s, 1H), 5.47 (s, 2H), 3.85-3.60 (m, 1H), 3.54-3.42 (m, 4H), 3.28-3.11 (m, 1H), 3.03-2.82 (m, 1H), 2.74-2.24 (m, 2H), 2.08 (s, 1H), 1.99-1.76 (m, 1H), 1.74-1.49 (m, 3H), 1.42-1.27 (m, 5H), 1.08-0.89 (m, 6H), 0.67 (dt, J=13.9, 6.7 Hz, 3H) ppm. MS: M/e 474 (M+1)⁺.

### Compound A351: 2-(7-((2S,5R)-4-(1-(6-(difluoromethyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile

### Step A: 1-(6-(difluoromethyl)pyridin-3-yl)ethan-1-one

A mixture of 5-bromo-2-(difluoromethyl)pyridine (1 g, 4.83 mmol), tributyl(1-ethoxyvinyl)stannane (2.1 g, 5.79 mmol) and Pd(PPh₃)₂Cl₂ (339 mg, 0.48 mmol) in toluene (10 mL) was stirred at 100° C. under N₂ overnight. The solution was added HCl (0.3 mL, 4 M in 1,4-dioxane) and the mixture was stirred at room temperature for 0.5 hour. The reaction mixture was diluted with EA and concentrated to dryness. The resulting residue was purified by flash column chromatography to give the titled compound (520 mg, 63%). MS: M/e 172 (M+1)⁺.

### Step B: 1-(6-(difluoromethyl)pyridin-3-yl)ethan-1-ol

To a solution of 1-(6-(difluoromethyl)pyridin-3-yl)ethan-1-one (150 mg, 0.87 mmol) in MeOH (15 mL) was added NaBH₄ (33 mg, 0.87 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (120 mg, 79%). MS: M/e 174 (M+1)⁺.

### Step C: 2-(7-((2S,5R)-4-(1-(6-(difluoromethyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile

A solution of Intermediate 5 (50 mg, 0.15 mmol), 1-(6-(difluoromethyl)pyridin-3-yl)ethan-1-ol (26 mg, 0.15 mmol), (cyanomethyl)trimethylphosphonium iodide (111 mg, 0.46 mmol) and DIPEA (98 mg, 0.76 mmol) in CH₃CN (3 ml) was stirred at 100° C. overnight. After completed, the solution was concentrated under reduced pressure to give the titled Compound A351, which was further separated into Compound A351a (24 mg) and Compound A351b (20 mg) by Prep-HPLC (Method A).

Compound A351a (the earlier peak): ¹H NMR (400 MHz, CD₃OD) δ 8.63 (s, 1H), 8.04 (d, J=7.7 Hz, 1H), 7.92 (s, 1H), 7.70 (d, J=7.7 Hz, 1H), 6.73 (t, J=55.1 Hz, 1H), 5.56 (s, 1H), 5.46 (s, 2H), 4.87-4.12 (m, 3H), 3.94 (d, J=6.5 Hz, 1H), 3.43 (s, 3H), 3.02 (d, J=12.1 Hz, 1H), 2.92 (d, J=10.8 Hz, 1H), 2.44-2.27 (m, 1H), 2.16-2.04 (m, 1H), 1.89-1.78 (m, 1H), 1.65-1.51 (m, 2H), 1.40 (d, J=6.5 Hz, 3H), 0.95 (t, J=7.2 Hz, 3H), 0.74 (t, J=7.1 Hz, 3H). MS: M/e 484 (M+1)⁺.

Compound A351b (the later peak): ¹H NMR (400 MHz, CD₃OD) δ 8.67 (s, 1H), 8.05 (d, J=7.8 Hz, 1H), 7.93 (s, 1H), 7.68 (d, J=7.9 Hz, 1H), 6.72 (t, J=55.3 Hz, 1H), 5.56 (s, 1H), 5.47 (s, 2H), 4.85-4.23 (m, 2H), 3.78 (d, J=6.1 Hz, 1H), 3.52 (d, J=13.0 Hz, 1H), 3.43 (s, 3H), 3.18 (d, J=9.0 Hz, 1H), 2.73 (d, J=10.9 Hz, 1H), 2.28 (d, J=11.9 Hz, 1H), 1.99-1.83 (m, 1H), 1.76-1.62 (m, 2H), 1.62-1.48 (m, 1H), 1.37 (d, J=6.1 Hz, 3H), 1.03 (t, J=6.7 Hz, 3H), 0.63 (t, J=7.1 Hz, 3H). MS: M/e 484 (M+1)⁺.

### Compound A352: 2-(7-((2S,5R)-4-(1-(3,3-dimethyl-2,3-dihydrofuro[3,2-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile

### Step A: 5-hydroxy-6-iodopicolinic acid

To a stirred solution of 5-hydroxypyridine-2-carboxylic acid (1 g, 6.8 mmol, 95%) in NH₃·H₂O (30 mL) were added a solution of KI (5.97 g, 34 mmol) and 12 (1.82 g, 6.8 mmol) in H₂O (40 mL) dropwise at room temperature. The resulting mixture was stirred for overnight at room temperature. The mixture was acidified to pH=3 with HCl (1M). The resulting mixture was extracted with EA (3×150 mL). The combined water layers were concentrated under reduced pressure to give the titled compound (1 g, 55%). MS: M/e 266 (M+1)⁺.

### Step B: methyl 5-hydroxy-6-iodopicolinate

To a stirred solution of 5-hydroxy-6-iodopyridine-2-carboxylic acid (1 g, 3.7 mmol) in MeOH was added SOCl₂ (1.3 mL, 18 mmol) dropwise at 0° C. The resulting mixture was stirred for 2 hours at 70° C. The mixture was allowed to cool down to room temperature. The resulting mixture was concentrated under reduced pressure. The residue was purified by reverse flash chromatography to give the titled compound (600 mg, 53%). MS: M/e 280 (M+1)⁺.

### Step C: methyl 6-iodo-5-((2-methylallyl)oxy)picolinate

A solution of methyl 5-hydroxy-6-iodopyridine-2-carboxylate (500 mg, 1.6 mmol), 3-bromo-2-methylprop-1-ene (250 mg, 1.8 mmol) and K₂CO₃ (609 mg, 4.1 mmol) in acetone was stirred at 60° C. overnight under nitrogen atmosphere. The resulting mixture was concentrated under reduced pressure. The residue was purified by flash column chromatography to give the titled compound (500 mg, 89%). MS: M/e 334 (M+1)⁺.

### Step D: methyl 3,3-dimethyl-2,3-dihydrofuro[3,2-b]pyridine-5-carboxylate

A solution of methyl 6-iodo-5-[(2-methylprop-2-en-1-yl)oxy]pyridine-2-carboxylate (500 mg, 1.5 mmol), n-Bu₃SnH (689 mg, 2.25 mmol) and AIBN (26 mg, 0.15 mmol) in benzene was stirred at 80° C. overnight under nitrogen atmosphere. The mixture was allowed to cool down to room temperature. The resulting mixture was added KF (10%, 5 mL) at room temperature. The resulting mixture was stirred for 3.5 hours at room temperature. The resulting mixture was extracted with EA (3×50 mL). The combined organic layers were dried over anhydrous Na₂SO₄. After filtration, the filtrate was concentrated under reduced pressure. The residue was purified by flash column chromatography to give the titled compound (240 mg, 77%). MS: M/e 208 (M+1)⁺.

### Step E: 3,3-dimethyl-2,3-dihydrofuro[3,2-b]pyridine-5-carboxylic acid

To a solution of methyl 3,3-dimethyl-2,3-dihydrofuro[3,2-b]pyridine-5-carboxylate (0.2 g, 0.97 mmol) in MeOH (4 mL)/water (1 mL) was added LiOH·H₂O (58 mg, 1.5 mmol). The mixture solution was stirred at room temperature for 5 hours. The reaction was quenched with water and acidified to pH=5-6 with citric acid, extracted with DCM (20 mL×2). The combined organic layers were washed with brine, dried over Na₂SO₄ and concentrated to give the titled compound (0.18 g, 96%). MS: M/e 194 (M+1)⁺.

### Step F: N-methoxy-N,3,3-trimethyl-2,3-dihydrofuro[3,2-b]pyridine-5-carboxamide

To a solution of 3,3-dimethyl-2,3-dihydrofuro[3,2-b]pyridine-5-carboxylic acid (0.14 g, 0.72 mmol) in DMF (2 mL) were added HATU (548 mg, 1.4 mmol), DIPEA (465 mg, 3.6 mol) and N,N-dimethyl-hydroxylamine hydrochloride salt (210 mg, 2.2 mol). The mixture was stirred at room temperature for overnight. The reaction was diluted with DCM and washed with water. The organic layer was separated, dried by Na₂SO₄, filtered, and concentrated to dryness. The resulting residue was purified by flash column chromatography to give the titled compound (120 mg, 70%). MS: M/e 237 (M+1)⁺.

### Step G: 1-(3,3-dimethyl-2,3-dihydrofuro[3,2-b]pyridin-5-yl)ethan-1-one

A mixture of N-methoxy-N,3,3-trimethyl-2,3-dihydrofuro[3,2-b]pyridine-5-carboxamide (120 mg, 0.51 mmol) in THF (5 mL) at 0° C. was added methyl magnesium bromide (0.33 mL, 3M, 1.02 mol). The reaction mixture was stirred at room temperature for 30 mins. The reaction was quenched by adding aqueous NH₄Cl.

The resulting mixture was extracted with EA, and then concentrated by using a rotary evaporator, to give the titled compound (110 mg). MS: M/e 192 (M+1)⁺.

### Step H: 1-(3,3-dimethyl-2,3-dihydrofuro[3,2-b]pyridin-5-yl)ethan-1-ol

To a solution of 1-(3,3-dimethyl-2,3-dihydrofuro[3,2-b]pyridin-5-yl)ethan-1-one (110 mg, 0.57 mmol) in MeOH (5 mL) was added NaBH₄ (21 mg, 0.57 mmol) at room temperature and the resulting mixture was stirred at room temperature for 5 min. The reaction mixture was diluted with DCM and washed with water, dried over Na₂SO₄ and concentrated to give the titled compound (100 mg). MS: M/e 194 (M+1)⁺.

### Step I: 2-(7-((2S,5R)-4-(1-(3,3-dimethyl-2,3-dihydrofuro[3,2-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile

To a solution of Intermediate 5 (30 mg, 0.09 mmol) in CH₃CN (1 mL) and was added 1-(3,3-dimethyl-2,3-dihydrofuro[3,2-b]pyridin-5-yl)ethan-1-ol (26 mg, 0.14 mmol), (cyanomethyl)trimethylphosphonium iodide (67 mg, 0.27 mmol) and DIPEA (118 mg, 0.9 mmol). The reaction was stirred at 105° C. overnight. The reaction solvent was removed under reduce pressure. The reaction mixture was diluted with DCM and washed with water. The organic layer was separated, dried over Na₂SO₄, filtered and concentrated to dryness, The resulting residue was purified by flash column chromatography and Prep-HPLC (Method A) to give the titled compound (3 mg). ¹H NMR (400 MHz, CD₃OD) δ 7.92 (d, J=3.1 Hz, 1H), 7.32 (dd, J=23.9, 8.4 Hz, 1H), 7.14 (dd, J=8.3, 5.3 Hz, 1H), 5.55 (s, 1H), 5.47 (d, J=3.5 Hz, 2H), 4.59 (s, 1H), 4.35 (s, 2H), 4.00-3.67 (m, 1H), 3.50 (d, J=14.1 Hz, 1H), 3.43 (s, 3H), 3.30-2.65 (m, 3H), 2.35 (dd, J=23.5, 11.2 Hz, 1H), 2.14-1.50 (m, 4H), 1.38 (d, J=1.8 Hz, 6H), 1.33 (d, J=6.6 Hz, 3H), 0.99 (dt, J=27.2, 7.4 Hz, 3H), 0.69 (dt, J=20.0, 7.3 Hz, 3H) ppm. MS: M/e 504 (M+1)⁺.

### Compound A354: 2-(7-((2S,5R)-2,5-diethyl-4-(1-(3-fluoro-5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile

### Step A: 3-fluoro-N-methoxy-N-methyl-5-(trifluoromethyl)picolinamide

A mixture of 3-fluoro-5-(trifluoromethyl)picolinic acid (418 mg, 2 mmol), N,O-dimethylhydroxylamine hydrochloride (234 mg, 2.4 mmol), HATU (917 mg, 2.4 mmol) and DIEPA (516 mg, 4 mmol) in CH₂Cl₂ (15 mL) was a stirred overnight. The reaction mixture was washed with brine, dried over Na₂SO₄ and concentrated to dryness. The resulting residue was purified by flash column chromatography to give the titled compound (430 mg, 85.3%). MS: M/e 253 (M+1)⁺.

### Step B: 1-(3-fluoro-5-(trifluoromethyl)pyridin-2-yl)ethan-1-one

To a stirred solution of 3-fluoro-N-methoxy-N-methyl-5-(trifluoromethyl)picolinamide (430 mg, 1.7 mmol) in dry THF (10 mL) was added dropwise MeMgBr (3.0 M, 0.6 mL, 1.79 mmol) at 0° C. After the, the mixture was stirred for half an hour. The reaction was quenched with aq·NH₄Cl, extracted with EtOAc (10 mL×3). The combined organic layers were washed with brine, dried over Na₂SO₄ and concentrated to dryness to give the titled compound (crude), which was used to the next step directly. MS: M/e 208 (M+1)⁺.

### Step C: 1-(3-fluoro-5-(trifluoromethyl)pyridin-2-yl)ethan-1-ol

To a stirred solution of 1-(3-fluoro-5-(trifluoromethyl)pyridin-2-yl)ethan-1-one (crude, 1.7 mmol) in MeOH (10 mL) was added NaBH₄ (65 mg, 1.7 mmol). After then, the mixture was stirred for 10 min. The reaction mixture was poured into H₂O (10 mL) and extracted with EtOAc (10 mL×3). The combined organic layers were washed with brine, dried over Na₂SO₄ and concentrated to dryness. The resulting residue was purified by flash column chromatography to give the titled compound (150 mg, 42%). MS: M/e 210 (M+1)⁺.

### Step D: 2-(7-((2S,5R)-2,5-diethyl-4-(1-(3-fluoro-5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile

A mixture of Intermediate 5 (32.8 mg, 0.1 mmol), 1-(3-fluoro-5-(trifluoromethyl)pyridin-2-yl)ethan-1-ol (31.3 mg, 0.15 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to give the titled Compound A354 (crude), which was purified by Prep-HPLC (Method A) to give Compound A354a (3 mg) and Compound A354b (4 mg).

Compound A354a (the earlier peak): ¹H NMR (400 MHz, CD₃OD) δ 8.74 (s, 1H), 7.99 (d, J=9.6 Hz, 1H), 7.92 (s, 1H), 5.52 (s, 1H), 5.47 (s, 2H), 4.35-4.25 (m, 1H), 3.57-3.47 (m, 1H), 3.43 (s, 3H), 3.35-3.25 (m, 2H), 3.15-2.85 (m, 2H), 2.46-2.37 (m, 1H), 1.93-1.79 (m, 1H), 1.70-1.50 (m, 3H), 1.46 (d, J=6.4 Hz, 3H), 0.99 (t, J=7.2 Hz, 3H), 0.66 (t, J=7.2 Hz, 3H) ppm. MS: M/e 520 (M+1)⁺.

Compound A354b (the later peak): ¹H NMR (400 MHz, CD₃OD) δ 8.73 (s, 1H), 7.99 (d, J=9.6 Hz, 1H), 7.92 (s, 1H), 5.53 (s, 1H), 5.47 (s, 2H), 4.51-4.41 (m, 1H), 3.43 (s, 3H), 3.38-3.25 (m, 4H), 3.17-3.08 (m, 1H), 2.86-2.77 (m, 1H), 1.83-1.56 (m, 4H), 1.52 (d, J=6.4 Hz, 3H), 0.91-0.76 (m, 6H) ppm. MS: M/e 520 (M+1)⁺

### Compound A362: 2-(7-((2S,5R)-2,5-diethyl-4-(1-(3-fluoro-5-(trifluoromethoxy)pyridin-2-yl)ethyl)piperazin-1-yl)-4-methyl-5-oxo-4,5-dihydro-2H-pyrazolo[4,3-b]pyridin-2-yl)acetonitrile

### Step A: methyl 5-(bromodifluoromethoxy)-3-fluoropicolinate

To a stirred solution of methyl 3-fluoro-5-hydroxypicolinate (1.71 g, 10 mmol) in NMP (20 mL) was added NaH (60%, 0.8 g, 20 mmol) at 0° C. After stirred for 30 min, dibromodifluoromethane (6.3 g, 30 mmol) was added and the mixture was stirred overnight. The reaction mixture was quenched with H₂O (30 mL), extracted with EtOAc (20 mL×3). The combined organic layers were washed with brine, dried over Na₂SO₄ and concentrated to dryness. The resulting residue was purified by flash column chromatography to give the titled compound (1.9 g, 63.3%). MS: M/e 300/302 (M+1)⁺.

### Step B: methyl 3-fluoro-5-(trifluoromethoxy)picolinate

To a solution of methyl 5-(bromodifluoromethoxy)-3-fluoropicolinate (1.9 g, 6.33 mmol) in CH₂Cl₂ (30 mL) was added AgBF₄ (1.84 g, 9.5 mmol) at −70° C. under N₂. After the addition, the reaction mixture was stirred overnight at RT. The reaction mixture was washed with H₂O, brine, dried over Na₂SO₄ and concentrated to dryness. The resulting residue was purified by flash column chromatography to give the titled compound (1.4 g, 92.5%). MS: M/e 240 (M+1)⁺.

### Step C: 3-fluoro-5-(trifluoromethoxy)picolinic acid

To a stirred solution of methyl 3-fluoro-5-(trifluoromethoxy)picolinate (478 mg, 2 mmol) in MeOH (5 mL) was added aq·NaOH (2.0 M, 2 mL). After the addition, the mixture was stirred for an hour. The reaction mixture was concentrated to give the residue, which was dissolved in H₂O and acidified to pH=3-4 with citric acid and extracted with EtOAc (15 mL×3). The combined organic layers were washed with brine, dried over Na₂SO₄ and concentrated to give the titled compound (400 mg, 88.9%). MS: M/e 226 (M+1)⁺.

Compound A362, and its separated isomers Compound A362a (12 mg) and A362b (14 mg) were prepared according to the similar procedures as described for Compound A354, Compound A354a and A354b using 3-fluoro-5-(trifluoromethoxy)picolinic acid under appropriate conditions that could be recognized by one skilled in the art.

Compound A362a (the earlier peak): ¹H NMR (400 MHz, CD₃OD) δ 8.45 (s, 1H), 7.92 (s, 1H), 7.73 (d, J=10.0 Hz, 1H), 5.52 (s, 1H), 5.47 (s, 2H), 4.23 (q, J=6.4 Hz, 1H), 3.54-3.46 (m, 1H), 3.42 (s, 3H), 3.32-3.30 (m, 2H), 3.12-3.05 (m, 1H), 2.92-2.83 (m, 1H), 2.43-2.33 (m, 1H), 1.94-1.79 (m, 1H), 1.68-1.47 (m, 3H), 1.45 (d, J=6.8 Hz, 3H), 0.99 (t, J=7.2 Hz, 3H), 0.64 (t, J=7.2 Hz, 3H) ppm. MS: M/e 536 (M+1)⁺.

Compound A362b (the later peak): ¹H NMR (400 MHz, CD₃OD) δ 8.45 (s, 1H), 7.92 (s, 1H), 7.73 (d, J=9.6 Hz, 1H), 5.53 (s, 1H), 5.47 (s, 2H), 4.39 (dd, J=13.2, 6.4 Hz, 1H), 3.43 (s, 3H), 3.37-3.33 (M, 1H), 3.32-3.30 (m, 2H), 3.15-3.05 (m, 1H), 2.85-2.77 (m, 1H), 2.60-2.49 (m, 1H), 1.85-1.53 (m, 4H), 1.50 (d, J=6.8 Hz, 3H), 0.82 (q, J=7.6 Hz, 6H) ppm. MS: M/e 536 (M+1)⁺

### Compound A368: 2-(7-((2S,5R)-4-(1-(2,6-naphthyridin-3-yl)ethyl)-2,5-diethylpiperazin-1-yl)-4-methyl-5-oxo-4,5-dihydro-2H-pyrazolo[4,3-b]pyridin-2-yl)acetonitrile

### Step A: 3-bromo-4-(dimethoxymethyl)pyridine

To a solution of 3-bromoisonicotinaldehyde (1 g, 5.4 mmol) in MeOH (5 mL) was added 4-methylbenzenesulfonic acid (1.1 g, 6.5 mol). The reaction mixture was stirred at 75° C. for 5 hours. The reaction was quenched by adding saturated solution of NaHCO₃. The resulting mixture was extracted with DCM, and then concentrated. The resulting residue was purified by flash column chromatography to give the titled compound (1.1 g, 88%). MS: M/e 232 (M+1)⁺.

### Step B: 4-(dimethoxymethyl)nicotinaldehyde

To a solution of 3-bromo-4-(dimethoxymethyl)pyridine (1.1 g, 4.7 mmol) in THF (15 mL) was added dropwise a solution of n-BuLi (mL, 1 mol) at −78° C. The reaction was stirred for 1 hour at −78° C., then was added DMF (1 g, 14.2 mmol). The reaction was stirred for 2 hours below −60° C., quenched by saturated solution of NH₄Cl. The resulting mixture was extracted with EA, and then concentrated. The resulting residue was purified by flash column chromatography to give the titled compound (0.5 g, 58%). MS: M/e 182 (M+1)⁺.

### Step C: methyl (E)-2-acetamido-3-(4-(dimethoxymethyl)pyridin-3-yl)acrylate

To a solution of 4-(dimethoxymethyl)nicotinaldehyde (500 mg, 2.7 mmol) in DCM (15 mL) was slowly added a solution of acetylamino-(dimethoxy-phosphoryl)-acetic acid methyl ester (717 mg, 3 mmol) and 1,8-diazabicyclo[5.4.0]undec-7-ene (456 mg, 3 mmol). The solution was stirred at 0° C. for 1 hour then at room temperature for 18 hours. The mixture was poured into water, extracted with dichloromethane, dried over sodium sulfate and concentrated to give the titled compound (600 mg). MS: M/e 295 (M+1)⁺.

### Step D: methyl 2,6-naphthyridine-3-carboxylate

A mixture of methyl (E)-2-acetamido-3-(4-(dimethoxymethyl)pyridin-3-yl)acrylate (600 mg, 2 mmol) in toluene (15 mL) was added 4-methylbenzenesulfonic acid (0.36 g, 1.8 mol). The reaction mixture was stirred at 110° C. for 18 hours. The brown residue was dissolved in EA, washed with saturated solution of NaHCO₃, dried over sodium sulfate and concentrated to give the titled compound (200 mg, 51%). MS: M/e 189 (M+1)⁺.

### Step E: 2,6-naphthyridine-3-carboxylic acid

To a solution of methyl 2,6-naphthyridine-3-carboxylate (0.2 g, 1.06 mmol) in MeOH (4 mL)/water (1 mL) was added LiOH·H₂O (64 mg, 1.6 mmol). The mixture solution was stirred at room temperature for 16 hours. The reaction was quenched with water and acidified to pH=5-6 with HCl (1M), extracted with (DCM:MeOH=10:1). The combined organic layers were dried over Na₂SO₄ and concentrated to give the titled compound (0.16 g, 86%). MS: M/e 175 (M+1)⁺.

Compound A368, and its separated isomers Compound A368a (2 mg) and A368b (1.5 mg) were prepared according to the similar procedures as described for Compound A354, Compound A354a and A354b using 2,6-naphthyridine-3-carboxylic acid under appropriate conditions that could be recognized by one skilled in the art.

Compound A368a (the earlier peak): ¹H NMR (400 MHz, CD₃OD) δ 9.39 (s, 2H), 8.65 (d, J=5.8 Hz, 1H), 8.19 (s, 1H), 8.03 (d, J=5.7 Hz, 1H), 7.93 (s, 1H), 5.57 (s, 1H), 5.47 (s, 2H), 4.82-4.15 (m, 2H), 4.00 (d, J=6.4 Hz, 1H), 3.60 (d, J=12.5 Hz, 1H), 3.44 (s, 3H), 3.22 (d, J=9.0 Hz, 1H), 2.83 (d, J=9.3 Hz, 1H), 2.39 (d, J=12.6 Hz, 1H), 2.17-1.55 (m, 4H), 1.47 (d, J=6.5 Hz, 3H), 1.06 (t, J=7.2 Hz, 3H), 0.59 (t, J=7.3 Hz, 3H) ppm. MS: M/e 485 (M+1)⁺.

Compound A368b (the later peak): ¹H NMR (400 MHz, CD₃OD) δ 9.41 (d, J=4.6 Hz, 2H), 8.65 (d, J=5.9 Hz, 1H), 8.13 (s, 1H), 8.03 (d, J=5.7 Hz, 1H), 7.92 (s, 1H), 5.56 (s, 1H), 5.45 (s, 2H), 4.80-4.16 (m, 2H), 4.20 (d, J=6.5 Hz, 1H), 3.43 (s, 3H), 3.41-3.35 (m, 1H), 3.04 (d, J=2.6 Hz, 2H), 2.49 (s, 1H), 2.20-1.56 (m, 4H), 1.51 (d, J=6.5 Hz, 3H), 0.96 (t, J=7.3 Hz, 3H), 0.71 (t, J=7.4 Hz, 3H) ppm. MS: M/e 485 (M+1)⁺.

### Compound A375: 2-(7-((2S,5R)-2,5-diethyl-4-(1-(4-((tetrahydrofuran-3-yl)oxy)phenyl)ethyl)piperazin-1-yl)-4-methyl-5-oxo-4,5-dihydro-2H-pyrazolo[4,3-b]pyridin-2-yl)acetonitrile

### Step A: 1-(4-((tetrahydrofuran-3-yl)oxy)phenyl)ethan-1-one

To a solution of 1-(4-hydroxyphenyl)ethan-1-one (272 mg, 2 mmol) in CH₃CN (5 mL) was added 3-iodotetrahydrofuran (440 mg, 2.2 mmol) and K₂CO₃ (552 mg, 4 mmol). The reaction mixture was stirred at 100° C. in a sealed bottle overnight. Another portion of 3-iodotetrahydrofuran (440 mg, 2.2 mmol) was added and stirred at 100° C. for 24 hours. The mixture was cooled down to rt, diluted with H₂O, extracted with EtOAc (80 mL), washed with brine, dried over Na₂SO₄, filtered, and concentrated to dryness. The resulting residue was purified by flash column chromatography to give the titled compound (250 mg, 60%). MS: M/e 207 (M+1)⁺.

### Step B: 1-(4-((tetrahydrofuran-3-yl)oxy)phenyl)ethan-1-ol

To a solution of 1-(4-((tetrahydrofuran-3-yl)oxy)phenyl)ethan-1-one (250 mg, 1.2 mmol) in MeOH (5 mL) was added NaBH₄ (46 mg, 1.2 mmol) slowly at 0° C. The resulting mixture was stirred at room temperature for 0.5 hour. The reaction mixture was quenched with H₂O (10 mL) and extracted with EtOAc (80 mL), washed with brine, dried over Na₂SO₄, filtered, and concentrated to dryness. The resulting residue was purified by flash column chromatography to give the titled compound (180 mg, 72%). ¹H NMR (400 MHz, DMSO-d₆) δ 7.24 (d, J=8.4 Hz, 2H), 6.84 (d, J=8.8 Hz, 2H), 5.04-4.94 (m, 2H), 4.70-4.60 (m, 1H), 3.95-3.69 (m, 4H), 2.26-2.10 (m, 1H), 1.99-1.88 (m, 1H), 1.28 (d, J=6.4 Hz, 3H) ppm.

### Step C: 2-(7-((2S,5R)-2,5-diethyl-4-(1-(4-((tetrahydrofuran-3-yl)oxy)phenyl)ethyl)piperazin-1-yl)-4-methyl-5-oxo-4,5-dihydro-2H-pyrazolo[4,3-b]pyridin-2-yl)acetonitrile

To a solution of 1-(4-((tetrahydrofuran-3-yl)oxy)phenyl)ethan-1-ol (42 mg, 0.2 mmol), Intermediate 5 (32 mg, 0.1 mmol) and (cyanomethyl) trimethyl phosphonium iodide (72 mg, 0.3 mmol) in CH₃CN (1 mL) was added DIPEA (64 mg, 0.5 mmol). The mixture was sealed in a bottle and heated at 100° C. for 16 hours. The mixture was cooled to room temperature, diluted with water, extracted with EtOAC (60 mL), washed with brine, dried over Na₂SO₄, filtered, and concentrated to dryness. The resulting residue was purified by Prep-HPLC (Method A) to give the titled compound (5 mg, 10%). ¹H NMR (400 MHz, CD₃OD) δ 7.93-7.89 (m, 1H), 7.34-7.24 (m, 2H), 6.92-6.82 (m, 2H), 5.54 (s, 1H), 5.46 (s, 2H), 5.04-4.96 (m, 1H), 4.02-3.80 (m, 4H), 3.75-3.45 (m, 2H), 3.43 (s, 3H), 3.35-3.31 (m, 1H), 3.21-2.78 (m, 2H), 2.65-2.05 (m, 4H), 2.04-1.45 (m, 4H), 1.36-1.26 (m, 3H), 1.05-0.90 (m, 3H), 0.75-0.55 (m, 3H) ppm. MS: M/e 519 (M+1)⁺.

### Compound A381: 2-(7-((2S,5R)-4-(1-(6-((4,4-difluoropiperidin-1-yl)methyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile

### Step A: methyl 6-((4,4-difluoropiperidin-1-yl)methyl)nicotinate

A solution of methyl 6-formylnicotinate (825 mg, 5 mmol), 4,4-difluoropiperidine (666 mg, 5.5 mmol) and AcOH (0.5 mL) in CH₂Cl₂ (15 mL) was stirred for 2 hours, then NaBH(OAc)₃ (2.12 g, 10 mmol) was added. After then, the reaction mixture was stirred overnight. The mixture was diluted with CH₂Cl₂ (20 mL). The organic layer was washed with H₂O, brine, dried over Na₂SO₄ and concentrated to dryness. The resulting residue was purified by flash column chromatography to give the titled compound (790 mg, 58.5%). MS: M/e 271 (M+1)⁺.

### Step B: 6-((4,4-difluoropiperidin-1-yl)methyl)nicotinic acid

To a stirred solution of methyl 6-((4,4-difluoropiperidin-1-yl)methyl)nicotinate (790 mg, 2.93 mmol) in MeOH (5 mL) was added aq·NaOH (4.0 M, 2 mL). After then, the mixture was stirred for 2 hours. The mixture was concentrated to give the aqueous layer, which was acidified to pH=3-4 with aq·HCl, then extracted with CH₂Cl₂/IPA (3/1, 30 mL×6). The combined organic layers were washed with brine, dried over Na₂SO₄ and concentrated to give the titled compound (606 mg, 80.8%). MS: M/e 257 (M+1)⁺.

Compound A381, and its separated isomers Compound A381a (3 mg) and A381b (6 mg) were prepared according to the similar procedures as described for Compound A354, Compound A354a and A354b using 6-((4,4-difluoropiperidin-1-yl)methyl)nicotinic acid under appropriate conditions that could be recognized by one skilled in the art.

Compound A381a (the earlier peak): ¹H NMR (400 MHz, CD₃OD) δ 8.49 (s, 1H), 7.92 (s, 1H), 7.89 (d, J=8.0 Hz, 1H), 7.55 (d, J=8.0 Hz, 1H), 5.56 (s, 1H), 5.46 (s, 2H), 3.92-3.85 (m, 1H), 3.80 (s, 2H), 3.43 (s, 3H), 3.35-3.25 (m, 3H), 3.05-2.87 (m, 2H), 2.77-2.65 (m, 4H), 2.38 (d, J=7.6 Hz, 1H), 2.16-1.98 (m, 5H), 1.89-1.51 (m, 3H), 1.39 (d, J=6.4 Hz, 3H), 0.95 (t, J=7.2 Hz, 3H), 0.72 (t, J=7.2 Hz, 3H) ppm. MS: M/e 567 (M+1)⁺.

Compound A381b (the later peak): ¹H NMR (400 MHz, CD₃OD) δ 8.52 (s, 1H), 7.92 (s, 1H), 7.90 (d, J=8.0 Hz, 1H), 7.52 (d, J=8.0 Hz, 1H), 5.55 (s, 1H), 5.47 (s, 2H), 3.74 (s, 2H), 3.72-3.67 (m, 1H), 3.55-3.46 (m, 1H), 3.43 (s, 3H), 3.35-3.25 (m, 2H) 3.21-3.13 (m, 1H), 2.73-2.57 (m, 5H), 2.30 (d, J=12.4 Hz, 1H), 2.08-1.91 (m, 5H), 1.72-1.52 (m, 3H), 1.36 (d, J=6.4 Hz, 3H), 1.03 (t, J=7.2 Hz, 3H), 0.60 (t, J=7.2 Hz, 3H) ppm. MS: M/e 567 (M+1)⁺.

### Compound A383: 2-(7-((2S,5R)-2,5-diethyl-4-(1-(4-(morpholinomethyl)phenyl)ethyl)piperazin-1-yl)-4-methyl-5-oxo-4,5-dihydro-2H-pyrazolo[4,3-b]pyridin-2-yl)acetonitrile

### Step A: 4-(4-bromobenzyl)morpholine

A solution of 4-bromobenzaldehyde (1 g, 5.41 mmol), morpholine (0.56 g, 6.44 mmol) and STAB (2.3 g, 10.85 mmol) in DCM (15 ml) was stirred at rt overnight. The solution was washed with brine (10 ml), dried over Na₂SO₄ and concentrated to dryness. The resulting residue was purified by flash column chromatography with 15-30% EtOAc in PE to give the titled compound (1 g, 72%). MS: M/e 256,258 (M+1)⁺.

### Step B: 1-(4-(morpholinomethyl)phenyl)ethan-1-one

A solution of 4-(4-bromobenzyl)morpholine (1 g, 3.91 mmol), ethyl tributylstannanecarboxylate (2.1 g, 5.82 mmol) and Pd(PPh₃)₂Cl₂ (137 mg, 0.20 mmol) in toluene (15 ml) was stirred at 100° C. overnight. The solution was cooled to RT. HCl/1,4-dioxane (4M, 2 ml) was added and stirred at RT for 10 min. The solution was diluted with EtOAc (15 ml), washed with aq·Na₂CO₃ (10 ml×2), dried over Na₂SO₄ and concentrated to dryness. The resulting residue was purified by flash column chromatography with 0-10% MeOH in DCM to give the titled compound (530 mg, crude). MS: M/e 220 (M+1)⁺.

### Step C: 1-(4-(morpholinomethyl)phenyl)ethan-1-ol

A solution of 1-(4-(morpholinomethyl)phenyl)ethan-1-one (530 mg, 2.42 mmol) and NaBH₄ (92 mg, 2.42 mmol) in MeOH (5 ml) was stirred at RT for 15 min. The solution was diluted with EtOAc (20 ml), washed with brine (10 ml×2), dried over Na₂SO₄ and concentrated to dryness to give the titled compound (530 mg, crude). MS: M/e 222 (M+1)⁺.

### Step D: 2-(7-((2S,5R)-2,5-diethyl-4-(1-(4-(morpholinomethyl)phenyl)ethyl)piperazin-1-yl)-4-methyl-5-oxo-4,5-dihydro-2H-pyrazolo[4,3-b]pyridin-2-yl)acetonitrile

A solution of Intermediate 5 (50 mg, 0.15 mmol), 1-(4-(morpholinomethyl)phenyl)ethan-1-ol (67.4 mg, 0.30 mmol), (cyanomethyl)trimethylphosphonium iodide (111 mg, 0.46 mmol) and DIPEA (197 mg, 1.53 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A383 (20 mg), which was further separated into Compound A383a (6 mg) and Compound A383b (7 mg) by Prep-HPLC (Method A).

Compound A383a (the earlier peak): ¹H NMR (400 MHz, DMSO-d₆): δ 7.97 (s, 1H), 7.28 (q, J=8.0 Hz, 4H), 5.60 (s, 2H), 5.38 (s, 1H), 3.67 (q, J=6.3 Hz, 1H), 3.57 (t, 4H), 3.44 (s, 2H), 3.30 (m, 2H), 3.26 (s, 3H), 3.10 (d, J=12.1 Hz, 1H), 2.90 (d, J=11.5 Hz, 1H), 2.74 (d, J=9.3 Hz, 1H), 2.34 (s, 5H), 2.09-1.94 (m, 1H), 1.69-1.54 (m, 1H), 1.49-1.37 (m, 2H), 1.27 (d, J=6.3 Hz, 3H), 0.86 (t, J=7.2 Hz, 3H), 0.58 (t, J=7.2 Hz, 3H) ppm. MS: M/e 532 (M+1)⁺.

Compound A383b (the later peak): ¹H NMR (400 MHz, DMSO-d₆): δ 7.98 (s, 1H), 7.32 (d, J=7.9 Hz, 2H), 7.24 (d, J=7.9 Hz, 2H), 5.61 (s, 2H), 5.37 (s, 1H), 3.60-3.51 (m, 5H), 3.44 (s, 2H), 3.31 (m, 2H), 3.27 (s, 3H), 3.04 (d, J=9.6 Hz, 1H), 2.48 (s, 2H), 2.32 (s, 4H), 2.22 (d, J=12.0 Hz, 1H), 1.93-1.79 (m, 1H), 1.64-1.36 (m, 3H), 1.24 (d, J=6.3 Hz, 3H), 0.94 (t, J=7.2 Hz, 3H), 0.48 (t, J=6.7 Hz, 3H) ppm. MS: M/e 532 (M+1)⁺.

### Compound A390: 2-(but-2-yn-1-yl)-7-((2S,5R)-4-(1-(2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one

### Step A: 5-bromo-2,2-dimethylbenzo[d][1,3]dioxole

To a mixture of 4-bromobenzene-1,2-diol (7.6 g, 40.2 mmol) and acetone (4.7 g, 80.4 mmol) in toluene (50 mL) was added PBr₃ (4.4 g, 16.3 mmol) and the resulted mixture was stirred at room temperature for 30 minutes. The mixture was quenched with NaHCO₃ (20 mL), extracted with EtOAc (50 mL×2). The extracts were combined and washed with brine (50 mL×2), dried over Na₂SO₄, filtered and concentrated to dryness.

The resulted residue was purified by column chromatography to give the titled compound (6.6 g, 72%). ¹H NMR (400 MHz, DMSO-d6) δ 7.07 (d, J=1.6 Hz, 1H), 6.96 (dd, J=8.4, 2.0 Hz, 1H), 6.80 (d, J=8.4 Hz, 1H), 1.64 (s, 7H).

### Step B: 1-(2,2-dimethylbenzo[d][1,3]dioxol-5-yl)ethan-1-ol

To a −70° C. solution of 5-bromo-2,2-dimethylbenzo[d][1,3]dioxole (6.1 g, 26.6 mmol) in THF (60 mL) was added n-BuLi (1.6 M, 18.5 mL, 29.6 mmol) in drops under N₂. After stirring for 30 minutes at −70° C., a solution of acetaldehyde (2.4 g, 54.5 mmol) in THF (5 mL) was added. Then the mixture was allowed warm to room temperature and stirred for 30 minutes. Aqueous solution of NH₄Cl (50 mL) was added and the mixture was extracted with EtOAc (50 mL×2), the organics were combined and washed with brine (50 mL×2), dried over Na₂SO₄, filtered and concentrated to dryness. The resulted residue was purified by column chromatography to give the titled compound (4.3 g, 83%). ¹H NMR (400 MHz, DMSO-d6) δ 6.79 (s, 1H), 6.76-6.67 (m, 2H), 5.01 (d, J=4.4 Hz, 1H), 4.68-4.52 (m, 1H), 1.61 (s, 6H), 1.27 (d, J=6.4 Hz, 3H).

### Step C: tert-butyl (2S,5R)-4-(1-(2,2-dimethylbenzo[d][1,3]dioxol-5-yl)ethyl)-2,5-dimethylpiperazine-1-carboxylate

To a mixture of tert-butyl (2S,5R)-2,5-dimethylpiperazine-1-carboxylate (220 mg, 1.0 mmol), 1-(2,2-dimethylbenzo[d][1,3]dioxol-5-yl)ethan-1-ol (200 mg, 1.0 mmol) and (cyanomethyl)trimethylphosphonium iodide (610 mg, 2.5 mmol) in CH₃CN (2 mL) was added DIPEA (500 mg, 3.9 mmol). The mixture was stirred at 100° C. in a sealed tube for 16 hours. The mixture was cooled and diluted with EtOAc (20 mL), washed with brine (10 mL×3), dried over Na₂SO₄, filtered and concentrated under reduced pressure. The resulting residue was purified by flash column chromatography to give the titled compound (250 mg, 64%). MS: M/e 391 (M+1)⁺.

### Step D: (2R,5S)-1-(1-(2,2-dimethylbenzo[d][1,3]dioxol-5-yl)ethyl)-2,5-dimethylpiperazine

To a solution of tert-butyl (2S,5R)-4-(1-(2,2-dimethylbenzo[d][1,3]dioxol-5-yl)ethyl)-2,5-dimethylpiperazine-1-carboxylate (150 mg, 0.38 mmol) in EA (1.0 mL) was added HCl/Dioxane (4 M, 2 mL) and the mixture was stirred at rt for 20 hours. The mixture was concentrated to dryness to give the titled compound (125 mg, 99%). MS: M/e 291 (M+1)⁺.

### Step E: 7-((2S,5R)-4-(1-(2,2-dimethylbenzo[d][1,3]dioxol-5-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

A mixture of (2R,5S)-1-(1-(2,2-dimethylbenzo[d][1,3]dioxol-5-yl)ethyl)-2,5-dimethylpiperazine (100 mg, 0.3 mmol), Intermediate 2 (131 mg, 0.34 mmol) and DIPEA (175 mg, 1.35 mmol) in DMAc (1 mL) was stirred at 100° C. for 16 hours. The mixture was concentrated under high vacuum and diluted with EA (20 mL), washed with brine (10 mL×2), dried over Na₂SO₄, filtered and concentrated under reduced pressure. The resulting residue was purified by flash column chromatography to give the titled compound (110 mg, 70%). MS: M/e 522 (M+1)⁺.

### Step F: 7-((2S,5R)-4-(1-(2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one

To a solution of 7-((2S,5R)-4-(1-(2,2-dimethylbenzo[d][1,3]dioxol-5-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (110 mg, 0.21 mmol) in EtOAc (1.0 mL) was added HCl/1,4-Dioxane (4 M, 1 mL) and the mixture was stirred at rt for 16 hours. The mixture was concentrated to dryness to give the titled compound (76 mg, 82%). MS: M/e 438 (M+1)⁺.

### Step G: 2-(but-2-yn-1-yl)-7-((2S,5R)-4-(1-(2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one

To a mixture of 7-((2S,5R)-4-(1-(2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one (76 mg, 0.17 mmol), K₂CO₃ (71 mg, 0.51 mmol) and H₂O (100 mg, 5.5 mmol) in DMF (1 mL) was added 1-bromobut-2-yne (35 mg, 0.26 mmol) at rt and the mixture was stirred for 6 hours. The mixture was diluted with EtOAc (10 mL), washed with brine (5 mL×3), dried over Na₂SO₄, filtered and concentrated under reduced pressure. The resulting residue was purified by flash column chromatography and Prep-HPLC (Method A) to give the titled compound (19 mg, 23%). ¹H NMR (400 MHz, CD₃OD) δ 7.90 (s, 1H), 6.83 (s, 1H), 6.80-6.71 (m, 1H), 6.71-6.61 (m, 1H), 5.53 (s, 1H), 5.05 (s, 2H), 4.75-4.17 (m, 1H), 3.76-3.35 (m, 6H), 3.08-2.94 (m, 1H), 2.91-2.82 (m, 1H), 2.81-2.29 (m, 1H), 1.87 (s, 3H), 1.68-1.61 (m, 6H), 1.38-1.18 (m, 6H), 1.17-0.96 (m, 3H). MS: M/e 490 (M+1)⁺.

### Compound A393: 2-(but-2-yn-1-yl)-7-((2S,5R)-5-ethyl-4-(1-(isoquinolin-3-yl)ethyl)-2-methylpiperazin-1-yl)-4-methyl-2,4-dihydro-5H-pyrazolo[4,3-b]pyridin-5-one

### Step A: 1-(isoquinolin-3-yl)ethan-1-ol

To a solution of 1-(isoquinolin-3-yl)ethan-1-one (200 mg, 1.17 mmol) in MeOH (8 mL) was added NaBH₄ (45 mg, 1.17 mmol) at room temperature and the resulting mixture was stirred at room temperature for 5 min. The reaction mixture was diluted with DCM and washed with water, dried over Na₂SO₄ and concentrated to give the titled compound (200 mg). MS: M/e 174 (M+1)⁺.

### Step B: 2-(but-2-yn-1-yl)-7-((2S,5R)-5-ethyl-4-(1-(isoquinolin-3-yl)ethyl)-2-methylpiperazin-1-yl)-4-methyl-2,4-dihydro-5H-pyrazolo[4,3-b]pyridin-5-one

To a solution of Intermediate 5 (50 mg, 0.15 mmol) in CH₃CN (2 mL) and was added 1-(isoquinolin-3-yl)ethan-1-ol (80 mg, 0.46 mmol), (cyanomethyl)trimethylphosphonium iodide (110 mg, 0.45 mmol) and DIPEA (197 mg, 1.5 mmol). The resulting mixture was stirred at 105° C. overnight. The reaction solvent was removed under reduce pressure, The reaction was diluted with DCM and washed with water. The organic layer was separated, dried by Na₂SO₄, filtered, and concentrated to dryness. The resulting residue was purified by Prep-HPLC (Method A) to give the titled compound (27 mg). ¹H NMR (400 MHz, CD₃OD) δ 9.22 (d, J=11.3 Hz, 1H), 8.14-8.07 (m, 1H), 8.02-7.88 (m, 3H), 7.82-7.63 (m, 2H), 5.55 (s, 1H), 5.03 (dd, J=12.1, 2.2 Hz, 2H), 4.61 (s, 1H), 4.18-3.91 (m, 1H), 3.64-3.54 (m, 1H), 3.44 (s, 3H), 3.39 (d, J=10.3 Hz, 1H), 3.25-3.01 (m, 1H), 2.91 (d, J=3.9 Hz, 1H), 2.56-2.22 (m, 1H), 1.84 (d, J=9.2 Hz, 3H), 1.80-1.55 (m, 2H), 1.49 (dd, J=10.1, 6.7 Hz, 3H), 1.34 (dd, J=55.6, 6.5 Hz, 3H), 1.08-0.69 (m, 3H) ppm. MS: M/e 483 (M+1)⁺.

### Compound A394: 2-(but-2-yn-1-yl)-7-((2S,5R)-5-ethyl-2-methyl-4-(1-(quinoxalin-2-yl)ethyl)piperazin-1-yl)-4-methyl-2,4-dihydro-5H-pyrazolo[4,3-b]pyridin-5-one

### Step A: 1-(quinoxalin-2-yl)ethan-1-ol

A mixture of quinoxaline-2-carbaldehyde (1 g, 6.3 mmol) in THF (20 mL) at 0° C. was added methyl magnesium bromide (3.2 mL, 3M, 9.5 mol). The reaction mixture was stirred at room temperature for 30 mins. The reaction was quenched by adding saturated solution of NH₄Cl. The resulting mixture was extracted with EtOAc. Then the resulting organic layer was concentrated to give the titled compound (1 g), which was used to next step directly. MS: M/e 175 (M+1)⁺.

### Step B: 2-(but-2-yn-1-yl)-7-((2S,5R)-5-ethyl-2-methyl-4-(1-(quinoxalin-2-yl)ethyl) piperazin-1-yl)-4-methyl-2,4-dihydro-5H-pyrazolo[4,3-b]pyridin-5-one

To a solution of Intermediate 11 (50 mg, 0.15 mmol) in CH₃CN (2 mL) and was added 1-(quinoxalin-2-yl)ethan-1-ol (80 mg, 0.46 mmol), (cyanomethyl)trimethylphosphonium iodide (110 mg, 0.45 mmol) and DIPEA (197 mg, 1.5 mmol). The resulting mixture was stirred at 105° C. overnight. The reaction solvent was removed under reduce pressure, The reaction was diluted with DCM and washed with water. The organic layer was separated, dried by Na₂SO₄, filtered, and concentrated to dryness. The resulting residue was purified by flash column chromatography to give the titled Compound A394 (50 mg), which was further separated into Compound A394a (7 mg) and Compound A394b (8 mg) by Prep-HPLC (Method A).

Compound A394a (the earlier peak): ¹H NMR (400 MHz, CD₃OD) δ 9.18 (s, 1H), 8.08 (t, J=9.1 Hz, 2H), 7.90 (s, 1H), 7.84 (p, J=8.5 Hz, 2H), 5.53 (s, 1H), 5.09 (s, 1H), 5.04 (d, J=2.4 Hz, 2H), 4.42 (s, 1H), 4.09-4.00 (m, 1H), 3.61 (d, J=13.8 Hz, 1H), 3.44 (s, 3H), 3.22 (d, J=10.3 Hz, 1H), 2.94-2.88 (m, 1H), 2.18 (d, J=12.7 Hz, 1H), 1.86 (s, 3H), 1.80-1.57 (m, 2H), 1.53 (d, J=6.8 Hz, 3H), 1.21 (d, J=6.5 Hz, 3H), 1.08 (t, J=7.3 Hz, 3H) ppm. MS: M/e 484 (M+1)⁺.

Compound A394b (the later peak): ¹H NMR (400 MHz, CD₃OD) δ 9.12 (s, 1H), 8.14-8.06 (m, 2H), 7.92-7.79 (m, 3H), 5.56 (s, 1H), 5.02 (d, J=2.4 Hz, 2H), 4.78-4.57 (m, 2H), 4.26 (d, J=6.5 Hz, 1H), 3.44 (s, 3H), 3.37 (s, 1H), 3.16-3.06 (m, 1H), 2.87 (d, J=10.7 Hz, 1H), 2.48 (s, 1H), 1.84 (d, J=2.3 Hz, 3H), 1.73-1.61 (m, 2H), 1.55 (d, J=6.7 Hz, 3H), 1.36 (d, J=6.5 Hz, 3H), 0.78 (t, J=7.4 Hz, 3H) ppm. MS: M/e 484 (M+1)⁺.

The following compounds were prepared according to the similar procedures under appropriate conditions:

| Compound | Name | Yield | Characterization |
|---|---|---|---|
| A3 | 7-((2S,5R)-2,5-dimethyl-4-(1-(quinoxalin- | 6 mg, 46% | ¹HNMR (400 MHz, CD₃OD) δ 8.92-8.84 (m, 2H), 8.20-7.96 (m, 3H), 7.77 (s, 1H), |
| | 6-yl)ethyl)piperazin-1- | | 5.53 (s, 1H), 5.10-4.86 (m, 0.5H), 4.80-4.45 (m, 1H), 4.40-4.35 (m, 0.5H), 4.12- |
| | yl)-2,4-dimethyl-2,4-dihydro-5H- | | 4.04 (m, 0.5H), 4.02 (s, 3H), 3.92-3.80 (m, 0.5H), 3.76-3.62 (m, 1H), 3.52-3.44 |
| | pyrazolo[4,3-b]pyridin-5-one | | (m, 0.5H), 3.43 (s, 3H), 3.21-2.79 (m, 2H), 2.35-2.10 (m, 0.5H), 1.60-1.36 (m, |
| | | | 4H), 1.32-0.99 (m, 5H) ppm. MS: M/e 432 (M + 1)⁺. |
| A4 | 7-((2S,5R)-2,5-dimethyl-4-(1- | 8 mg, 53% | ¹HNMR (400 MHz, CD₃OD) δ 8.91-8.80 (m, 2H), 8.16-8.00 (m, 3H), 7.93 (d, J = |
| | (quinoxalin-6-yl)ethyl)piperazin-1- | | 1.6 Hz, 1H), 5.56 (s, 1H), 5.14-5.02 (m, 2H), 4.75-4.50 (m, 1.5H), 4.40-4.30 (m, |
| | yl)-4-methyl-2-(2,2,2- | | 0.5H), 4.05-3.77 (m, 1H), 3.73-3.64 (m, 1H), 3.52-3.45 (m, 0.5H), 3.44 (s, 3H), |
| | trifluoroethyl)-2,4-dihydro-5H- | | 3.16-2.80 (m, 2H), 2.26-2.14 (m, 0.5H), 1.55-1.38 (m, 4H), 1.28-1.16 (m, 4H), |
| | pyrazolo[4,3-b]pyridin-5-one | | 1.05 (d, J = 6.0 Hz, 1H) ppm. MS: M/e 500 (M + 1)⁺. |
| A5 | 7-((2S,5R)-2,5-dimethyl-4-(1- | 5 mg, 35% | ¹HNMR (400 MHz, CD₃OD) δ 8.90-8.83 (m, 2H), 8.16-7.99 (m, 3H), 7.81 (s, 1H), |
| | (quinoxalin-6-yl)ethyl)piperazin-1- | | 5.53 (s, 1H), 4.75-4.50 (m, 1.5H), 4.45-4.35 (m, 2.5H), 4.05-3.90 (m, 0.5H), 3.88- |
| | yl)-2-(2-methoxyethyl)-4-methyl- | | 3.75 (m, 2.5H), 3.72-3.64 (m, 1H), 3.51-3.44 (m, 0.5H), 3.44 (s, 3H), 3.31 (s, 3H), |
| | 2,4-dihydro-5H-pyrazolo[4,3- | | 3.16-3.04 (m, 0.5H), 2.97-2.80 (m, 1.5H), 2.25-2.10 (m, 0.5H), 1.52-1.39 (m, |
| | b]pyridin-5-one | | 4H), 1.26-1.16 (m, 4H), 1.06 (d, J = 6.4 Hz, 1H) ppm. MS: M/e 476 (M + 1)⁺. |
| A6 | 2-(7-((2S,5R)-2,5-dimethyl-4-(1- | 4 mg, 27% | ¹HNMR (400 MHz, CD₃OD) δ 8.92-8.82 (m, 2H), 8.19-7.99 (m, 3H), 7.85 (s, 1H), |
| | (quinoxalin-6-yl)ethyl)piperazin-1- | | 5.55 (s, 1H), 4.99 (d, J = 2.0 Hz, 2H), 4.75-4.35 (m, 2H), 4.05-3.64 (m, 2H), 3.51- |
| | yl)-4-methyl-5-oxo-4,5-dihydro-2H- | | 3.44 (m, 0.5H), 3.45 (s, 3H), 3.21-2.75 (m, 2H), 2.26-2.10 (m, 0.5H), 1.60-1.39 |
| | pyrazolo[4,3-b]pyridin-2- | | (m, 4H), 1.26-1.13 (m, 4H), 1.12-1.00 (m, 1H) ppm. MS: M/e 475 (M + 1)⁺· |
| | yl)acetamide | | |
| A7 | methyl 2-(7-((2S,5R)-2,5-dimethyl- | | ¹H NMR (400 MHz, CD₃OD) δ 8.87 (d, J = 3.8 Hz, 2H), 8.14-8.01 (m, 3H), 7.87 (s, |
| | 4-(1-(quinoxalin-6- | | 1H), 5.56 (s, 1H), 5.04 (s, 2H), 4.90 (s, 1H), 4.29 (s, 1H), 3.97 (q, J = 6.5 Hz, 1H), 3.74 |
| | yl)ethyl)piperazin-1-yl)-4-methyl-5- | | (s, 3H), 3.45 (d, J = 8.0 Hz, 4H), 3.08 (dd, J = 20.0, 11.6 Hz, 1H), 2.92 (d, J = 12.5 Hz, |
| | oxo-4,5-dihydro-2H-pyrazolo[4,3- | | 2H), 1.43 (dd, J = 6.3, 4.0 Hz, 6H), 1.04 (d, J = 6.5 Hz, 3H). MS: M/e 490. (M + 1)⁺. |
| | b]pyridin-2-yl)acetate | | |
| A8 | 2-(7-((2S,5R)-2,5-dimethyl-4-(1- | 4 mg, 28% | ¹HNMR (400 MHz, CD₃OD) δ 8.90-8.84 (m, 2H), 8.20-7.96 (m, 4H), 5.82-5.71 |
| | (quinoxalin-6-yl)ethyl)piperazin-1- | | (m, 1H), 5.57 (d, J = 2.8 Hz, 1H), 4.75-4.25 (m, 2H), 4.10-3.79 (m, 1H), 3.76-3.65 |
| | yl)-4-methyl-5-oxo-4,5-dihydro-2H- | | (m, 1H), 3.51-3.44 (m, 0.5H), 3.44 (s, 3H), 3.19-2.80 (m, 2H), 2.28-2.16 (m, 0.5H), |
| | pyrazolo[4,3-b]pyridin-2- | | 1.95-1.88 (m, 3H), 1.55-1.38 (m, 4H), 1.26-1.16 (m, 4H), 1.14-1.03 (m, 1H). |
| | yl)propanenitrile | | MS: M/e 471 (M + 1)⁺. |
| A9 | 7-((2S,5R)-2,5-dimethyl-4-(1- | 3 mg, 21% | ¹HNMR (400 MHz, CD₃OD) δ 8.90-8.84 (m, 2H), 8.15-7.99 (m, 3H), 7.83 (s, 1H), |
| | (quinoxalin-6-yl)ethyl)piperazin-1- | | 5.53 (s, 1H), 4.75-4.50 (m, 2H), 4.38-4.29 (m, 2H), 4.02-3.89 (m, 2H), 3.87-3.78 |
| | yl)-2-(2-hydroxyethyl)-4-methyl- | | (m, 0.5H), 3.73-3.62 (m, 1.5H), 3.50-3.44 (m, 0.5H), 3.44 (s, 3H), 3.15-3.04 (m, |
| | 2,4-dihydro-5H-pyrazolo[4,3- | | 0.5H), 2.97-2.80 (m, 1.5H), 2.25-2.14 (m, 0.5H), 1.51-1.37 (m, 4H), 1.26-1.17 |
| | b]pyridin-5-one | | (m, 4H), 1.05 (d, J = 6.4 Hz, 1H) ppm. MS: M/e 462 (M + 1)⁺. |
| A10 | 2-(cyclopropylmethyl)-7-((2S,5R)- | 8 mg, 56% | ¹HNMR (400 MHz, CD₃OD) δ 8.91-8.82 (m, 2H), 8.17-7.95 (m, 3H), 7.86 (s, 1H), |
| | 2,5-dimethyl-4-(1-(quinoxalin-6- | | 5.53 (s, 1H), 4.77-4.50 (m, 2H), 4.19-4.02 (m, 2H), 4.02-3.76 (m, 1H), 3.74-3.62 |
| | yl)ethyl)piperazin-1-yl)-4-methyl- | | (m, 1H), 3.51-3.44 (m, 0.5H), 3.45 (s, 3H), 3.17-2.76 (m, 2H), 2.26-2.14 (m, 0.5H), |
| | 2,4-dihydro-5H-pyrazolo[4,3- | | 1.55-1.39 (m, 4H), 1.36-1.15 (m, 5H), 1.13-1.01 (m, 1H), 0.66-0.57 (m, 2H), |
| | b]pyridin-5-one | | 0.48-0.37 (m, 2H) ppm. MS: M/e 472 (M + 1)⁺. |
| A11 | 7-((2S,5R)-2,5-dimethyl-4-(1- | 7 mg, 47% | ¹HNMR (400 MHz, CD₃OD) δ 8.91-8.83 (m, 2H), 8.15-8.00 (m, 3H), 7.86 (s, 1H), |
| | (quinoxalin-6-yl)ethyl)piperazin-1- | | 5.53 (s, 1H), 4.75-4.55 (m, 1.5H), 4.45-4.25 (m, 2.5H), 4.01-3.92 (m, 0.5H), 3.87- |
| | yl)-2-(2-(dimethylamino)ethyl)-4- | | 3.78 (m, 0.5H), 3.72-3.64 (m, 1H), 3.50-3.44 (m, 0.5H), 3.44 (s, 3H), 3.15-3.02 |
| | methyl-2,4-dihydro-5H- | | (m, 0.5H), 2.97-2.80 (m, 3.5H), 2.29 (s, 6H), 2.24-2.17 (m, 0.5H), 1.51-1.38 (m, |
| | pyrazolo[4,3-b]pyridin-5-one | | 4H), 1.26-1.17 (m, 4H), 1.05 (d, J = 6.0 Hz, 1H) ppm. MS: M/e 489 (M + 1)⁺. |
| A12 | 7-((2S,5R)-2,5-dimethyl-4-(1- | 4 mg, 28% | ¹HNMR (400 MHz, CD₃OD) δ 8.90-8.84 (m, 2H), 8.15-7.99 (m, 3H), 7.83 (s, 1H), |
| | (quinoxalin-6-yl)ethyl)piperazin-1- | | 5.54 (s, 1H), 4.75-4.56 (m, 2.5H), 4.40-4.15 (m, 1.5H), 4.05-3.76 (m, 1H), 3.74- |
| | yl)-4-methyl-2-(oxiran-2-ylmethyl)- | | 3.60 (m, 1H), 3.51-3.44 (m, 0.5H), 3.44 (s, 3H), 3.41-3.34 (m, 1H), 3.15-3.04 (m, |
| | 2,4-dihydro-5H-pyrazolo[4,3- | | 0.5H), 3.00-2.75 (m, 2.5H), 2.62-2.55 (m, 1H), 2.26-2.14 (m, 0.5H), 1.52-1.39 |
| | b]pyridin-5-one | | (m, 4H), 1.26-1.15 (m, 4H), 1.06 (d, J = 6.4 Hz, 1H) ppm. MS: M/e 474 (M + 1)⁺. |
| A13 | 7-((2S,5R)-2,5-dimethyl-4-(1- | 3 mg, 20% | ¹HNMR (400 MHz, DMSO-d₆) δ 9.03-8.96 (m, 2H), 8.50 (s, 1H), 8.22-7.99 (m, |
| | (quinoxalin-6-yl)ethyl)piperazin-1- | | 3H), 5.61 (s, 1H), 5.02-4.20 (m, 2H), 4.04-3.82 (m, 1H), 3.67 (s, 3H), 3.64-3.58 |
| | yl)-4-methyl-2-(methylsulfonyl)- | | (m, 1H), 3.41-3.38 (m, 0.5H), 3.37 (s, 3H), 3.11-2.70 (m, 2H), 2.21-2.00 (m, 0.5H), |
| | 2H-pyrazolo[4,3-b]pyridin-5(4H)- | | 1.50-1.35 (m, 4.5H), 1.22-1.14 (m, 3H), 1.08-1.01 (m, 1.5H) ppm. MS: M/e 496 |
| | one | | (M + 1)⁺. |
| A14 | 2-cyclobutyl-7-((2S,5R)-2,5- | 6 mg, 42% | ¹HNMR (400 MHz, CD₃OD) δ 8.90-8.84 (m, 2H), 8.15-7.99 (m, 3H), 7.87 (s, 1H), |
| | dimethyl-4-(1-(quinoxalin-6- | | 5.52 (s, 1H), 5.00-4.87 (m, 1.5H), 4.75-4.35 (m, 1.5H), 4.02-3.79 (m, 1H), 3.73- |
| | yl)ethyl)piperazin-1-yl)-4-methyl- | | 3.62 (m, 1H), 3.50-3.44 (m, 0.5H), 3.43 (s, 3H), 3.13-3.04 (m, 0.5H), 2.98-2.81 |
| | 2,4-dihydro-5H-pyrazolo[4,3- | | (m, 1.5H), 2.70-2.42 (m, 4H), 2.25-2.14 (m, 0.5H), 1.96-1.80 (m, 2H), 1.51-1.38 |
| | b]pyridin-5-one | | (m, 4H), 1.27-1.17 (m, 4H), 1.06 (d, J = 6.4 Hz, 1H) ppm. MS: M/e 472 (M + 1)⁺ |
| A15 | 2-(cyclopropanecarbonyl)-7- | 5 mg, 34% | ¹HNMR (400 MHz, DMSO-d₆) δ 8.97-8.88 (m, 2H), 8.44 (s, 1H), 8.17-7.92 (m, |
| | ((2S,5R)-2,5-dimethyl-4-(1- | | 3H), 5.56 (s, 1H), 5.02-4.21 (m, 2H), 4.02-3.75 (m, 1H), 3.67-3.52 (m, 1H), 3.34- |
| | (quinoxalin-6-yl)ethyl)piperazin-1- | | 3.31 (m, 0.5H), 3.30 (s, 3H), 3.16-3.02 (m, 1H), 3.01-2.73 (m, 2H), 2.14-2.06 (m, |
| | yl)-4-methyl-2,4-dihydro-5H- | | 0.5H), 1.47-1.30 (m, 4.5H), 1.22-1.04 (m, 7H), 1.03-0.97 (m, 1.5H) ppm. MS: M/e |
| | pyrazolo[4,3-b]pyridin-5-one | | 486 (M + 1)⁺. |
| A18 | 7-((2S,5R)-2,5-dimethyl-4-(1- | 1 mg, 7% | ¹HNMR (400 MHz, CD₃OD) δ 8.91-8.84 (m, 2H), 8.17-8.00 (m, 3H), 7.85-7.84 |
| | (quinoxalin-6-yl)ethyl)piperazin-1- | | (m, 1H), 5.52 (s, 1H), 4.90-4.87 (m, 0.5H), 4.80-4.55 (m, 2H), 4.51-4.35 (m, 0.5H), |
| | yl)-2-isopropyl-4-methyl-2,4- | | 4.02-3.93 (m, 0.5H), 3.87-3.77 (m, 0.5H), 3.74-3.62 (m, 1H), 3.51-3.44 (m, 0.5H), |
| | dihydro-5H-pyrazolo[4,3-b]pyridin- | | 3.44 (s, 3H), 3.10-3.03 (m, 0.5H), 2.96-2.80 (m, 1.5H), 2.26-2.14 (m, 0.5H), 1.53 |
| | 5-one | | (t, J = 6.4 Hz, 6H), 1.49-1.40 (m, 4.5H), 1.27-1.16 (m, 3H), 1.08-1.02 (m, 1.5H) |
| | | | ppm. MS: M/e 460 (M + 1)⁺. |
| A21 | 7-((2S,5R)-2,5-dimethyl-4-(1- | 3 mg, 21% | ¹HNMR (400 MHz, CD₃OD) δ 8.89-8.84 (m, 2H), 8.14-7.99 (m, 3H), 7.91 (s, 1H), |
| | (quinoxalin-6-yl)ethyl)piperazin-1- | | 5.54 (s, 1H), 5.12 (t, J = 2.8 Hz, 2H), 5.00-4.90 (m, 0.5H), 4.75-4.20 (m, 1.5H), 4.01- |
| | yl)-4-methyl-2-(prop-2-yn-1-yl)- | | 3.93 (m, 0.5H), 3.86-3.78 (m, 0.5H), 3.72-3.60 (m, 1H), 3.50-3.44 (m, 0.5H), |
| | 2,4-dihydro-5H-pyrazolo[4,3- | | 3.44 (s, 3H), 3.12-3.00 (m, 1.5H), 2.97-2.80 (m, 1.5H), 2.25-2.16 (m, 0.5H), 1.51- |
| | b]pyridin-5-one | | 1.37 (m, 4.5H), 1.26-1.16 (m, 3H), 1.04 (d, J = 6.0 Hz, 1.5H) ppm. MS: M/e 456 |
| | | | (M + 1)⁺. |
| A27 | 7-((2S,5R)-2,5-diethyl-4-(4- | 2 mg, 13% | ¹HNMR (400 MHz, CD₃OD) δ 8.19 (s, 1H), 7.49 (d, J = 7.2 Hz, 2H), 7.23 (d, J = 7.2 |
| | (trifluoromethoxy)benzyl)piperazin- | | Hz, 2H), 5.58-5.50 (m, 2H), 4.93 (s, 1H), 4.56-4.33 (m, 1H), 3.80-3.70 (m, 1H), |
| | 1-yl)-4-methyl-2-(prop-1-en-2-yl)- | | 3.69-3.60 (m, 1H), 3.54-3.45 (m, 1H), 3.45 (s, 3H), 2.91-2.65 (m, 3H), 2.56-2.44 |
| | 2,4-dihydro-5H-pyrazolo[4,3- | | (m, 1H), 2.35 (s, 3H), 2.07-1.89 (m, 1H), 1.85-1.70 (m, 1H), 1.66-1.54 (m, 2H), |
| | b]pyridin-5-one | | 1.01-0.90 (m, 3H), 0.82-0.72 (m, 3H) ppm. MS: M/e 504 (M + 1)⁺. |
| A30 | 2-(7-((2S,5R)-4-(1-(4-fluoro-3- | 15 mg, 35% | ¹H NMR (400 MHz, DMSO-d6) δ 7.99 (s, 1H), 7.43 (s, 1H), 7.39-7.29 (m, 1H), 7.15 |
| | (methoxymethyl)phenyl)ethyl)-2,5- | | (dd, J = 19.8, 10.1 Hz, 1H), 5.62 (s, 2H), 5.40 (d, J = 5.6 Hz, 1H), 4.46 (d, J = 7.8 Hz, |
| | dimethylpiperazin-1-yl)-4-methyl-5- | | 2H), 3.66-3.45 (m, 2H), 3.31 (s, 2H), 3.31-3.29 (m, 2H), 3.27 (s, 3H), 3.06-2.94 |
| | oxo-4,5-dihydro-2H-pyrazolo[4,3- | | (m, 1H), 2.86 (s, 1H), 2.76 (d, J = 9.5 Hz, 1H), 2.71-2.56 (m, 1H), 1.28-1.22 (m, |
| | b]pyridin-2-yl)acetonitrile | | 5H), 1.07 (dd, J = 10.2, 6.4 Hz, 3H), 0.90 (d, J = 6.5 Hz, 1H) ppm. MS: M/e 467 |
| | | | (M + 1)+ |
| A31 | 2-(7-((2S,5R)-4-(1-(4- | 3.5 mg, 39% | ¹H NMR (400 MHz, CD₃OD) δ 7.92 (s, 1H), 7.40 (s, 2H), 7.31 (s, 2H), 5.56 (s, 1H), |
| | (methoxymethyl)phenyl)ethyl)-2,5- | | 5.47 (d, J = 2.3 Hz, 2H), 4.93 (s, 1H), 4.61 (s, 1H), 4.45 (d, J = 5.1 Hz, 2H), 3.63 (d, J = |
| | dimethylpiperazin-1-yl)-4-methyl-5- | | 11.6 Hz, 2H), 3.43 (s, 3H), 3.38 (d, J = 3.0 Hz, 3H), 3.0-2.4 (m, 3H), 1.42-1.32 (m, |
| | oxo-4,5-dihydro-2H-pyrazolo[4,3- | | 5H), 1.21 (d, J = 6.6 Hz, 2H), 1.16 (s, 1H), 1.00 (s, 1H) ppm. MS: M/e 449 (M + 1)⁺. |
| | b]pyridin-2-yl)acetonitrile | | |
| A32 | 2-(7-((2S,5R)-4-(1-(2,6-difluoro-4- | 7 mg, 32% | ¹H NMR (400 MHz, CD₃OD) δ 7.93 (d, J = 2.1 Hz, 1H), 7.28 (ddd, J = 22.4, 12.1, 6.6 |
| | methoxyphenyl)ethyl)-2,5- | | Hz, 1H), 6.92-6.81 (m, 1H), 5.57 (d, J = 3.0 Hz, 1H), 5.48 (d, J = 1.5 Hz, 2H), 4.60 |
| | dimethylpiperazin-1-yl)-4-methyl-5- | | (s, 1H), 4.37 (s, 0.5H), 4.03 (s, 0.5H), 3.86 (d, J = 4.4 Hz, 3H), 3.61 (d, J = 12.7 Hz, |
| | oxo-4,5-dihydro-2H-pyrazolo[4,3- | | 1H), 3.44 (s, 3H), 2.94 (m, 1H), 2.80 (d, J = 9.2 Hz, 2H), 2.23 (d, J = 12.6 Hz, 1H), |
| | b]pyridin-2-yl)acetonitrile | | 1.40-1.29 (m, 5H), 1.24 (d, J = 6.6 Hz, 1H), 1.14 (d, J = 6.4 Hz, 2H), 1.02 (d, J = 6.5 |
| | | | Hz, 1H) ppm. MS: M/e 471 (M + 1)⁺. |
| A33 | 2-(7-((2S,5R)-4-(1-(5-fluoropyridin- | 6 mg | ¹H NMR (400 MHz, CD₃OD) δ 8.38 (s, 1H), 7.93 (s, 1H), 7.65 (d, J = 8.9 Hz, 2H), |
| | 2-yl)ethyl)-2,5-dimethylpiperazin-1- | | 5.57 (s, 1H), 5.47 (s, 2H), 4.54-4.50 (m, 0.5H), 3.90 (s, 0.5H), 3.65 (d, J = 14.5 Hz, |
| | yl)-4-methyl-5-oxo-4,5-dihydro-2H- | | 2H), 3.46 (d, J = 16.7 Hz, 4H), 3.11-2.77 (m, 3H), 1.34 (dd, J = 21.7, 16.1 Hz, 5H), |
| | pyrazolo[4,3-b]pyridin-2- | | 1.25-1.13 (m, 3H), 1.05 (s, 1H) ppm. MS: M/e 424(M + 1)⁺. |
| | yl)acetonitrile | | |
| A34 | 2-(7-((2S,5R)-4-(1- | 6 mg | ¹H NMR (400 MHz, CD₃OD) δ 8.78 (d, J = 15.3 Hz, 1H), 8.39 (s, 1H), 7.90 (d, J = |
| | ([1,2,4]triazolo[1,5-a]pyridin-6- | | 20.5 Hz, 2H), 7.78 (t, J = 9.2 Hz, 1H), 5.57 (s, 1H), 5.47 (s, 2H), 4.66 (d, J = 26.6 Hz, |
| | yl)ethyl)-2,5-dimethylpiperazin-1- | | 1H), 3.77-3.59 (m, 2H), 3.44 (s, 3H), 3.14-2.80 (m, 3H), 2.27 (d, J = 11.8 Hz, 1H), |
| | yl)-4-methyl-5-oxo-4,5-dihydro-2H- | | 1.43 (d, J = 11.9 Hz, 4H), 1.20 (d, J = 11.6 Hz, 4H), 1.08 (s, 1H) ppm. MS: M/e |
| | pyrazolo[4,3-b]pyridin-2- | | 446(M + 1)⁺. |
| | yl)acetonitrile | | |
| A35 | 2-(7-((2S,5R)-4-(1-(2,5-difluoro-4- | 2 mg, 11% | ¹H NMR (400 MHz, CD₃OD) δ 7.92 (s, 1H), 6.52 (d, J = 12.6 Hz, 2H), 5.55 (s, 1H), |
| | methoxyphenyl)ethyl)-2,5- | | 5.48 (s, 2H), 4.58 (d, J = 52.1 Hz, 1H), 4.03 (s, 1H), 3.78 (s, 3H), 3.55 (d, J = 10.0 Hz, |
| | dimethylpiperazin-1-yl)-4-methyl-5- | | 2H), 3.45 (s, 1H), 3.43 (s, 3H), 2.87 (d, J = 11.2 Hz, 1H), 2.25 (d, J = 12.0 Hz, 1H), |
| | oxo-4,5-dihydro-2H-pyrazolo[4,3- | | 1.50 (s, 3H), 1.17 (d, J = 3.1 Hz, 3H), 1.09 (s, 3H) ppm. MS: M/e 471(M + 1)⁺. |
| | b]pyridin-2-yl)acetonitrile | | |
| A36 | 2-(7-((2S,5R)-4-(1-(4- | 3 mg, 24% | ¹H NMR (400 MHz, CD₃OD) δ 7.92 (s, 1H), 7.20 (s, 2H), 7.05 (d, J = 10.6 Hz, 2H), |
| | cyclopropylphenyl)propyl)-2,5- | | 5.54 (s, 1H), 5.47 (s, 2H), 4.37 (s, 1H), 3.62 (s, 1H), 3.41 (d, J = 18.4 Hz, 4H), 2.92 (s, |
| | dimethylpiperazin-1-yl)-4-methyl-5- | | 1H), 2.78 (m, 1H), 2.26 (s, 1H), 1.94 (s, 1H), 1.90 (s, 1H), 1.32 (d, J = 23.0 Hz, 3H), |
| | oxo-4,5-dihydro-2H-pyrazolo[4,3- | | 1.20 (s, 2H), 1.12 (s, 2H), 0.96 (s, 3H), 0.67 (s, 5H) ppm. MS: M/e 459(M + 1)⁺. |
| | b]pyridin-2-yl)acetonitrile | | |
| A37 | 2-(7-((2S,5R)-2,5-dimethyl-4-(1- | 2 mg, 37% | ¹H NMR (400 MHz, CD₃OD) δ 7.92 (s, 1H), 7.38 (s, 2H), 7.31 (d, J = 7.7 Hz, 2H), |
| | phenylethyl)piperazin-1-yl)-4- | | 7.24 (s, 1H), 5.56 (s, 1H), 5.47 (s, 2H), 4.6 (s, 1H), 4.2 (s, 1H), 3.63 (d, J = 9.7 Hz, 2H), |
| | methyl-5-oxo-4,5-dihydro-2H- | | 3.48 (s, 1H), 3.43 (s, 3H), 2.99 (s, 2H), 1.32 (d, J = 17.3 Hz, 6H), 1.18 (d, J = 25.7 Hz, |
| | pyrazolo[4,3-b]pyridin-2- | | 3H) ppm. MS: M/e 405.2 (M + 1)⁺. |
| | yl)acetonitrile | | |
| A38 | 2-(7-((2S,5R)-4-(1-(4- | 1 mg, 18% | ¹H NMR (400 MHz, CD₃OD) δ 7.93 (s, 1H), 7.30 (s, 2H), 6.88 (s, 2H), 5.56 (s, 1H), |
| | methoxyphenyl)ethyl)-2,5- | | 5.47 (s, 2H), 4.30-4.26 (m, 0.5H), 3.90 (s, 0.5H), 3.78 (s, 3H), 3.60 (s, 2H), 3.48 (s, |
| | dimethylpiperazin-1-yl)-4-methyl-5- | | 2H), 3.43 (s, 3H), 2.75 (s, 2H), 1.33 (s, 3H), 1.29 (s, 3H), 1.17 (d, J = 22.6 Hz, 3H) |
| | oxo-4,5-dihydro-2H-pyrazolo[4,3- | | ppm. MS: M/e 435.2 (M + 1)⁺. |
| | b]pyridin-2-yl)acetonitrile | | |
| A39 | 2-(7-((2S,5R)-4-(1-(isoquinolin-7- | 14 mg, 41% | ¹H NMR (400 MHz, DMSO-d6) δ 9.29 (d, J = 11.8 Hz, 1H), 8.48 (s, 1H), 8.05 (d, J = |
| | yl)ethyl)-2,5-dimethylpiperazin-1- | | 11.6 Hz, 1H), 8.02-7.92 (m, 2H), 7.88 (s, 1H), 7.81 (s, 1H), 5.61 (s, 2H), 5.41 (s, 1H), |
| | yl)-4-methyl-5-oxo-4,5-dihydro-2H- | | 3.67-3.42 (m, 2H), 3.28 (s, 3H), 2.95 (d, J = 9.7 Hz, 3H), 2.86-2.71 (m, 2H), 1.36 |
| | pyrazolo[4,3-b]pyridin-2- | | (s, 3H), 1.25-0.92 (m, 6H) ppm. MS: M/e 456 (M + 1)+. |
| | yl)acetonitrile | | |
| A40 | 2-(7-((2S,5R)-2,5-dimethyl-4-(1- | 20 mg, 47% | ¹H NMR (400 MHz, DMSO-d6) δ 8.88 (s, 1H), 8.34 (s, 1H), 8.03-7.90 (m, 3H), 7.71 |
| | (quinolin-7-yl)ethyl)piperazin-1-yl)- | | (s, 1H), 7.50 (s, 1H), 5.62 (s, 2H), 5.40 (s, 1H), 3.92-3.48 (m, 3H), 3.28 (s, 3H), 2.94 |
| | 4-methyl-5-oxo-4,5-dihydro-2H- | | (s, 2H), 2.87-2.66 (m, 2H), 1.36 (s, 3H), 1.29-0.90 (m, 6H). MS: M/e 456 (M + 1)⁺ |
| | pyrazolo[4,3-b]pyridin-2- | | |
| | yl)acetonitrile | | |
| A41 | 2-(7-((2S,5R)-2,5-dimethyl-4-(1-(2- | 9 mg, 39% | ¹H NMR (400 MHz, CD₃OD) δ 7.92 (s, 1H), 7.65 (s, 1H), 7.58-7.35 (m, 2H), 5.56 (s, |
| | methylbenzo[d]oxazol-5- | | 1H), 5.47 (s, 2H), 4.72-4.05 (m, 2H), 3.85-3.57 (m, 2H), 3.43 (s, 3H), 3.05-2.72 |
| | yl)ethyl)piperazin-1-yl)-4-methyl-5- | | (m, 2H), 2.64 (s, 3H), 2.22 (d, J = 11.2 Hz, 1H), 1.45-1.31 (m, 5H), 1.24-1.13 (m, |
| | oxo-4,5-dihydro-2H-pyrazolo[4,3- | | 3H), 1.04-0.96 (m, 1H) ppm. MS: M/e 460 (M + 1)⁺. |
| | b]pyridin-2-yl)acetonitrile | | |
| A42 | 2-(7-((2S,5R)-2,5-dimethyl-4-(1-(2- | 4 mg, 6% | ¹H NMR (400 MHz, CD₃OD) δ 7.97-7.89 (m, 1H), 7.55-7.41 (m, 2H), 7.39-7.29 |
| | methylbenzo[d]oxazol-7- | | (m, 1H), 5.56 (s, 1H), 5.51-5.43 (m, 2H), 4.88-4.28 (m, 2H), 4.26-4.02 (m, 1H), |
| | yl)ethyl)piperazin-1-yl)-4-methyl-5- | | 3.73-3.57 (m, 1H), 3.43 (s, 3H), 3.43-3.37 (m, 1H), 3.12-3.04 (m, 0.5H), 2.89- |
| | oxo-4,5-dihydro-2H-pyrazolo[4,3- | | 2.79 (m, 1H), 2.70-2.61 (m, 3H), 2.25-2.19 (m, 0.5H), 1.47 (t, J = 6.0 Hz, 3H), 1.39 |
| | b]pyridin-2-yl)acetonitrile | | (d, J = 6.4 Hz, 1.5H), 1.24-1.12 (m, 3H), 1.03 (d, J = 6.4 Hz, 1.5H) ppm. MS: M/e |
| | | | 460 (M + 1)⁺. |
| A43 | 2-(7-((2S,5R)-4-(1-(2,3- | 9.0 mg, 27% | A43: ¹H NMR (400 MHz, CD₃OD) δ 7.93 (s, 1H), 6.95-6.70 (m, 3H), 5.56 (s, 1H), |
| | dihydrobenzo[b][1,4]dioxin-6- | | 5.48 (s, 2H), 4.70-4.49 (m, 1H), 4.27-4.19 (m, 4H), 3.70-3.35 (m, 6H), 3.05-2.65 |
| | yl)ethyl)-2,5-dimethylpiperazin-1- | | (m, 3H), 1.40-0.99 (m, 9H) ppm. MS: M/e 463 (M + 1)⁺. |
| | yl)-4-methyl-5-oxo-4,5-dihydro-2H- | | A43a, separated isomer (the earlier peak): ¹H NMR (400 MHz, DMSO-d₆) δ 8.15 (s, |
| | pyrazolo[4,3-b]pyridin-2- | | 1H), 7.98 (s, 1H), 6.87-6.75 (m, 3H), 5.61 (s, 2H), 5.40 (s, 1H), 4.48 (s, 1H), 4.21 (s, |
| | yl)acetonitrile | | 4H), 3.45 (d, J = 12.1 Hz, 2H), 3.35 (d, J = 6.4 Hz, 2H), 3.27 (s, 3H), 2.59 (dd, J = 11.8, |
| | | | 3.7 Hz, 1H), 2.15 (d, J = 11.8 Hz, 1H), 1.22 (d, J = 6.5 Hz, 3H), 1.09 (d, J = 6.5 Hz, |
| | | | 3H), 1.03 (d, J = 6.5 Hz, 3H) ppm. MS: M/e 463 (M + 1)⁺. |
| | | | A43b, separated isomer (the later peak): ¹H NMR (400 MHz, DMSO-d₆) δ 8.18 (s, 1H), |
| | | | 7.98 (s, 1H), 6.83 (d, J = 13.4 Hz, 3H), 5.62 (s, 2H), 5.39 (s, 1H), 4.23 (s, 4H), 3.47 (q, |
| | | | J = 6.5 Hz, 2H), 3.27 (s, 3H), 3.24-3.19 (m, 2H), 2.87-2.80 (m, 2H), 2.73 (d, J = 10.1 |
| | | | Hz, 1H), 1.24 (d, J = 6.6 Hz, 3H), 1.20 (d, J = 6.4 Hz, 3H), 0.89 (d, J = 6.5 Hz, 3H) |
| | | | ppm. MS: M/e 463 (M + 1)⁺. |
| A44 | 2-(7-((2S,5R)-2,5-dimethyl-4- | 18 mg, 46% | ¹H NMR (400 MHz, CD₃OD) δ 8.87 (s, 2H), 8.14-8.04 (m, 2H), 8.05-7.96 (m, 1H), |
| | (quinoxalin-6-ylmethyl)piperazin-1- | | 7.97-7.89 (m, 1H), 5.60 (s, 1H), 5.48 (s, 2H), 4.83-4.70 (m, 1H), 4.64-4.41 (m, |
| | yl)-4-methyl-5-oxo-4,5-dihydro-2H- | | 1H), 3.98 (d, J = 15.2 Hz, 1H), 3.83 (d, J = 14.4 Hz, 1H), 3.67 (d, J = 13.2 Hz, 1H), |
| | pyrazolo[4,3-b]pyridin-2- | | 3.44 (s, 3H), 3.24 (s, 1H), 3.05 (d, J = 11.2 Hz, 1H), 2.43 (d, J = 10.8 Hz, 1H), 1.35 (s, |
| | yl)acetonitrile | | 3H), 1.20 (s, 3H) ppm. MS: M/e 443 (M + 1)⁺. |
| A45 | 2-(7-((2S,5R)-2,5-dimethyl-4-(1-(2- | 7 mg, 21% | ¹H NMR (400 MHz, CD₃OD) δ 7.93 (s, 1H), 7.74-7.21 (m, 3H), 5.57 (s, 1H), 5.47 (s, |
| | methylbenzo[d]oxazol-4- | | 2H), 4.74-3.55 (m, 4H), 3.43 (s, 3H), 3.05-2.75 (m, 2H), 2.65 (s, 3H), 2.34-2.08 |
| | yl)ethyl)piperazin-1-yl)-4-methyl-5- | | (m, 1H), 1.55-0.96 (m, 9H) ppm. MS: M/e 460 (M + 1)⁺. |
| | oxo-4,5-dihydro-2H-pyrazolo[4,3- | | |
| | b]pyridin-2-yl)acetonitrile | | |
| A48 | 2-(7-((2S,5R)-2,5-diethyl-4-(1- | 12 mg, 53% | ¹H NMR (400 MHz, DMSO-d6) δ 9.28 (s, 1H), 8.49 (s, 1H), 8.11 (s, 1H), 7.98 (s, 1H), |
| | (isoquinolin-6-yl)ethyl)piperazin-1- | | 7.90 (d, J = 9.1 Hz, 1H), 7.85-7.71 (m, 2H), 5.61 (s, 2H), 5.40 (s, 1H), 4.06-3.64 |
| | yl)-4-methyl-5-oxo-4,5-dihydro-2H- | | (m, 2H), 3.27 (s, 3H), 3.16-2.79 (m, 5H), 1.51 (s, 2H), 1.38-1.31 (m, 3H), 1.24 (s, |
| | pyrazolo[4,3-b]pyridin-2- | | 2H), 1.00-0.86 (m, 3H), 0.62-0.41 (m, 3H) ppm. MS: M/e 484 (M + 1)⁺ |
| | yl)acetonitrile | | |
| A49 | 2-(7-((2S,5R)-4-(1- | 4 mg | ¹H NMR (400 MHz, CD₃OD) δ 8.75 (s, 1H), 8.39 (s, 1H), 7.93 (s, 1H), 7.76 (d, J = |
| | ([1,2,4]triazolo[1,5-a]pyridin-7- | | 15.7 Hz, 1H), 7.38 (s, 1H), 5.57 (s, 1H), 5.47 (s, 2H), 3.91 (d, J = 62.8 Hz, 1H), 3.43 |
| | yl)ethyl)-2,5-diethylpiperazin-1-yl)- | | (s, 3H), 3.18 (s, 1H), 2.94 (dd, J = 74.4, 45.6 Hz, 2H), 2.50-2.33 (m, 1H), 2.14 (s, |
| | 4-methyl-5-oxo-4,5-dihydro-2H- | | 1H), 1.87 (s, 1H), 1.61 (s, 2H), 1.40 (s, 2H), 1.28 (s, 3H), 1.00 (d, J = 30.5 Hz, 3H), |
| | pyrazolo[4,3-b]pyridin-2- | | 0.70 (d, J = 35.8 Hz, 3H) ppm. MS: M/e 474(M + 1)⁺. |
| | yl)acetonitrile | | |
| A50 | 2-(7-((2S,5R)-2,5-diethyl-4-(1- | 19 mg, 37% | ¹H NMR (400 MHz, DMSO-d6) δ 7.98 (s, 1H), 7.94-7.80 (m, 4H), 7.59 (d, J = 7.4 |
| | (naphthalen-2-yl)ethyl)piperazin-1- | | Hz, 1H), 7.49 (s, 2H), 5.61 (s, 2H), 5.47-5.27 (m, 2H), 3.75 (s, 1H), 3.27 (s, 3H), 3.10 |
| | yl)-4-methyl-5-oxo-4,5-dihydro-2H- | | (s, 2H), 2.98 (s, 3H), 1.99 (s, 2H), 1.32 (s, 3H), 0.98 (s, 3H), 0.85 (s, 2H), 0.48 (s, 3H) |
| | pyrazolo[4,3-b]pyridin-2- | | ppm. MS: M/e 483 (M + 1)⁺ |
| | yl)acetonitrile | | |
| A51 | 2-(7-((2S,5R)-2,5-diethyl-4-(1- | 23 mg, 35% | ¹H NMR (400 MHz, DMSO-d6) δ 8.94 (s, 2H), 8.15-8.02 (m, 2H), 7.98 (s, 2H), 5.61 |
| | (quinoxalin-6-yl)ethyl)piperazin-1- | | (s, 2H), 5.40 (s, 1H), 4.07-3.87 (m, 1H), 3.44 (s, 1H), 3.21-2.92 (m, 2H), 2.86-2.61 |
| | yl)-4-(methyl-d3)-5-oxo-4,5- | | (m, 1H), 2.35-2.22 (m, 1H), 2.09-1.86 (m, 1H), 1.69-1.47 (m, 3H), 1.37 (d, J = |
| | dihydro-2H-pyrazolo[4,3-b]pyridin- | | 12.0 Hz, 3H), 1.29-1.07 (m, 1H), 0.95-0.91 (m, 3H), 0.55-0.52 (m, 3H) ppm. MS: |
| | 2-yl)acetonitrile | | M/e 488 (M + 1)+ |
| A53 | 2-(7-((2S,5R)-2,5-diethyl-4-((4- | 4 mg, 23% | ¹H NMR (400 MHz, CDCl₃) δ 8.21-8.13 (m, 0.5 H), 7.69-7.37 (m, 3H), 7.25-6.51 (m, |
| | fluoro-2-methoxyphenyl)(4- | | 4.5 H), 6.02-5.59 (m, 2H), 5.16-5.10 (m, 3H), 3.90-3.78 (m, 3H), 3.56-3.36 (m, 4H), |
| | fluorophenyl)methyl)piperazin-1- | | 3.32-3.13 (m, 3H), 2.67-2.55 (m, 2H), 2.25-1.76 (m, 2H), 1.26 (m, 1H), 0.88-0.61 (m, |
| | yl)-4-methyl-5-oxo-4,5-dihydro-2H- | | 6H) ppm. MS: M/e 561 (M + 1)⁺. |
| | pyrazolo[4,3-b]pyridin-2- | | |
| | yl)acetonitrile. | | |
| A54 | 2-(7-((2S,5R)-2,5-diethyl-4- | 13 mg, 16% | MS: M/e 471 (M + 1)⁺. ¹H NMR (400 MHz, CDCl₃) δ 8.94-8.90 (m, 1H), 8.84 (s, 1H), |
| | (quinoxalin-6-ylmethyl)piperazin-1- | | 8.23-8.05 (m, 2H), 7.92-7.85 (m, 1H), 7.39 (s, 1H), 5.64 (s, 1H), 5.19-5.14 (m, 2H), |
| | yl)-4-methyl-5-oxo-4,5-dihydro-2H- | | 4.68-4.37 (m, 2H), 3.94-3.82 (m, 2H), 3.52-3.50 (m, 1H), 3.42 (s, 3H), 3.05-2.74 (m, |
| | pyrazolo[4,3-b]pyridin-2- | | 2H), 2.51-2.49 (m, 1H), 2.01-1.60 (m, 4H), 1.20-1.16 (m, 1H), 0.92 (s, 3H), 0.72 (s, |
| | yl)acetonitrile. | | 3H) ppm. |
| A55 | 2-(7-((2S,5R)-2,5-diethyl-4-(1- | 1 mg, 18% | ¹H NMR (400 MHz, CD₃OD) δ 9.10 (s, 1H), 8.09 (s, 2H), 7.93 (s, 1H), 7.84 (s, 2H), |
| | (quinoxalin-2-yl)ethyl)piperazin-1- | | 5.57 (s, 1H), 5.46 (s, 2H), 4.26 (s, 1H), 3.43 (s, 4H), 3.01 (s, 2H), 2.85 (s, 2H), 2.48 (s, |
| | yl)-4-methyl-5-oxo-4,5-dihydro-2H- | | 1H), 1.67 (s, 2H), 1.55 (s, 2H), 1.29 (s, 3H), 1.06 (s, 1H), 0.87 (s, 2H), 0.76 (s, 2H), |
| | pyrazolo[4,3-b]pyridin-2- | | 0.58 (s, 1H) ppm. MS: M/e 485 (M + 1)⁺. |
| | yl)acetonitrile | | |
| A56 | 2-(7-((2S,5R)-2,5-diethyl-4-(1- | 5 mg, 53% | ¹H NMR (400 MHz, CD₃OD) δ 8.95 (d, J = 12.4 Hz, 1H), 8.30 (d, J = 7.3 Hz, 1H), |
| | (quinolin-3-yl)ethyl)piperazin-1-yl)- | | 8.02 (s, 1H), 7.94 (d, J = 11.8 Hz, 2H), 7.76 (s, 1H), 7.63 (s, 1H), 5.56 (s, 1H), 5.46 (s, |
| | 4-methyl-5-oxo-4,5-dihydro-2H- | | 2H), 4.08 (s, 0.5H), 3.91 (s, 0.5H), 3.57 (d, J = 13.1 Hz, 1H), 3.43 (s, 3H), 3.13 (s, 1H), |
| | pyrazolo[4,3-b]pyridin-2- | | 3.08-2.92 (m, 1H), 2.76 (d, J = 11.6 Hz, 2H), 2.48-2.34 (m, 1H), 2.13 (s, 1H), 1.85 |
| | yl)acetonitrile | | (s, 2H), 1.62 (s, 1H), 1.48 (d, J = 9.4 Hz, 3H), 1.03-0.97 (m, 3H), 0.7 (s, 1H), 0.7 (s, |
| | | | 2H) ppm. MS: M/e 484 (M + 1)⁺. |
| A57 | 2-(7-((2S,5R)-2,5-diethyl-4-(1- | 7 mg, 33% | ¹H NMR (400 MHz, CD₃OD) δ 9.23 (s, 1H), 8.10 (d, J = 7.3 Hz, 1H), 7.95 (d, J = 16.9 |
| | (quinolin-2-yl)ethyl)piperazin-1-yl)- | | Hz, 3H), 7.79 (s, 1H), 7.67 (s, 1H), 5.56 (s, 1H), 5.47 (s, 2H), 4.12 (s, 0.6H), 3.94 (s, |
| | 4-methyl-5-oxo-4,5-dihydro-2H- | | 0.4H), 3.59 (s, 1H), 3.43 (s, 4H), 3.06 (s, 1H), 2.81 (s, 1H), 2.47 (s, 1H), 2.12 (s, 1H), |
| | pyrazolo[4,3-b]pyridin-2- | | 1.85 (s, 2H), 1.62 (s, 2H), 1.49 (d, J = 11.0 Hz, 3H), 1.02 (d, J = 39.6 Hz, 3H), 0.64 (d, |
| | yl)acetonitrile | | J = 33.5 Hz, 3H) ppm. MS: M/e 484 (M + 1)⁺. |
| A58 | 2-(7-((2S,5R)-2,5-diethyl-4-(1- | 6 mg, 25% | ¹H NMR (400 MHz, CD₃OD) δ 8.39-8.35(m, 1H), 8.10 (d, J = 7.3 Hz, 1H), 7.95-7.81 |
| | (isoquinolin-3-yl)ethyl)piperazin-1- | | (m, 2H), 7.79-7.75 (m, 2H), 7.67-7.60 (m, 1H), 5.56 (s, 1H), 5.47 (s, 2H), 4.12 (s, 0.6H), |
| | yl)-4-methyl-5-oxo-4,5-dihydro-2H- | | 3.94 (s, 0.4H), 3.59 (s, 1H), 3.43 (s, 4H), 3.06 (s, 1H), 2.81 (s, 1H), 2.47 (s, 1H), 2.12 |
| | pyrazolo[4,3-b]pyridine-2- | | (s, 1H), 1.85 (s, 2H), 1.62 (s, 2H), 1.49 (d, J = 11.0 Hz, 3H), 1.02 (d, J = 39.6 Hz, 3H), |
| | yl)acetonitrile | | 0.64 (d, J = 33.5 Hz, 3H) ppm. MS: M/e 484 (M + 1)⁺. |
| A59 | 2-(7-((2S,5R)-2,5-diethyl-4-(1-(1- | 3 mg | ¹H NMR (400 MHz, CD3OD) δ 7.92 (s, 1H), 7.63-7.62 (m, 1H), 7.52-7.50 (m, 1H), |
| | methyl-1H-benzo[d]imidazol-2- | | 7.31-7.26 (m, 2H), 5.57-5.54 (m, 1H), 5.46 (s, 2H), 4.45-4.32 (m, 1H), 4.03 (s, |
| | yl)ethyl)piperazin-1-yl)-4-methyl-5- | | 3H), 3.65-3.54 (m, 1H), 3.43 (s, 3H), 3.17-3.10 (m, 0.5H), 2.98-2.94 (m, 1H), 2.85- |
| | oxo-4,5-dihydro-2H-pyrazolo[4,3- | | 2.80 (m, 0.5H), 2.53-2.49 (m, 0.5H), 2.42-2.30 (m, 0.5H), 2.21-2.16 (m, 0.5H), |
| | b]pyridine-2-yl)acetonitrile | | 2.06-1.98 (m, 0.5H), 1.89-1.87 (m, 1H), 1.63-1.57 (m, 4H), 1.37-1.36 (m, 1H), |
| | | | 1.05-1.03 (m, 1H), 0.92-0.75 (m, 6H), 0.64-0.59 (m, 1H) ppm. MS: M/e 487(M + 1)⁺ |
| A60 | 2-(7-((2S,5R)-2,5-diethyl-4-(1- | 11 mg, 59% | ¹H NMR (400 MHz, DMSO-d6) δ 8.87 (s, 1H), 8.34 (s, 1H), 8.06-7.93 (m, 2H), 7.93- |
| | (quinolin-6-yl)ethyl)piperazin-1-yl)- | | 7.90 (m, 1H), 7.88-7.83 (m, 1H), 7.55-7.50 (m, 1H), 5.61 (s, 2H), 5.40 (s, 1H), 3.96- |
| | 4-methyl-5-oxo-4,5-dihydro-2H- | | 3.79 (m, 1H), 3.47-3.35 (m, 1H), 3.27 (s, 3H), 3.20-2.69 (m, 5H), 2.33-2.24 (m, |
| | pyrazolo[4,3-b]pyridin-2- | | 1H), 2.10-1.91 (m, 1H), 1.68-1.45 (m, 3H), 1.40-1.30 (m, 3H), 0.88 (s, 2H), 0.62- |
| | yl)acetonitrile | | 0.38 (m, 3H) ppm. MS: M/e 484 (M + 1)⁺ |
| A61 | 2-(7-((2S,5R)-2,5-diethyl-4-(1- | 11 mg, 36% | ¹HNMR (400 MHz, CD₃OD) δ 8.83 (s, 1H), 8.40-8.36 (m, 1H), 8.10-7.85 (m, 3H), |
| | (quinolin-7-yl)ethyl)piperazin-1-yl)- | | 7.76 (t, J = 8.0 Hz, 1H), 7.58-7.48 (m, 1H), 5.56 (s, 1H), 5.45 (s, 2H), 4.08-3.96 (m, |
| | 4-methyl-5-oxo-4,5-dihydro-2H- | | 0.5H), 3.90-3.81 (m, 0.5H), 3.60-3.52 (m, 0.5H), 3.43 (s, 3H), 3.31-3.28 (m, 2H), |
| | pyrazolo[4,3-b]pyridin-2- | | 3.26-2.85 (m, 2H), 2.78-2.66 (m, 0.5H), 2.55-2.30 (m, 1H), 2.25-1.50 (m, 4H), |
| | yl)acetonitrile | | 1.49-1.38 (m, 3H), 1.10-0.93 (m, 3H), 0.72-0.49 (m, 3H) ppm. MS: M/e 484 |
| | | | (M + 1)⁺. |
| A64 | 2-(7-((2S,5R)-2,5-diethyl-4-(1-(1- | 3 mg, 20% | 1H NMR (400 MHz, CD3OD) δ 8.23 (s, 1H), 7.93 (s, 1H), 7.79-7.55 (m, 2H), 7.42 |
| | methyl-1H-benzo[d]imidazol-6- | | (s, 1H), 5.63-5.26 (m, 4H), 3.93 (s, 3H), 3.43 (s, 3H), 3.01-2.56 (m, 3H), 2.41-2.00 |
| | yl)ethyl)piperazin-1-yl)-4-methyl-5- | | (m, 3H), 1.83-1.43 (m, 5H), 1.22-0.77 (m, 5H), 0.79-0.49 (m, 3H) ppm. MS: M/e |
| | oxo-4,5-dihydro-2H-pyrazolo[4,3- | | 487 (M + 1)+. |
| | b]pyridin-2-yl)acetonitrile | | |
| A66 | 2-(7-((2S,5R)-4-(1-(2,2- | 15 mg, 46% | ¹H NMR (400 MHz, CD₃OD) δ 7.93 (s, 1H), 7.26-7.05 (m, 3H), 5.55 (s, 1H), 5.47 (s, |
| | difluorobenzo[d][1,3]dioxol-4- | | 2H), 3.87-3.67 (m, 1H), 3.51-3.44 (m, 1H), 3.43 (s, 3H), 2.97-2.92 (m, 1H), 2.75- |
| | yl)ethyl)-2,5-diethylpiperazin-1-yl)- | | 2.68 (m, 1H), 2.39-2.36 (m, 1H), 2.08-1.64 (m, 6H), 1.54-1.40 (m, 4H), 1.03- |
| | 4-methyl-5-oxo-4,5-dihydro-2H- | | 0.94 (m, 3H), 0.75-0.61 (m, 2H) ppm. MS: M/e 513 (M + 1)⁺. |
| | pyrazolo[4,3-b]pyridin-2- | | |
| | yl)acetonitrile | | |
| A67 | 2-(7-((2S,5R)-2,5-diethyl-4-(1-(4- | 4 mg, 23% | ¹H NMR (400 MHz, CDCl₃) δ 7.42-7.32 (m, 3H), 7.19-6.99 (m, 2H), 5.65-5.59 (m, |
| | fluorophenyl)ethyl)piperazin-1-yl)- | | 1H), 5.15-5.11 (m, 2H), 4.48-4.08 (m, 1H), 3.68-3.53 (m, 1H), 3.41 (s, 3H), 3.37-2.82 |
| | 4-methyl-5-oxo-4,5-dihydro-2H- | | (m, 3H), 2.80-2.56 (m, 1H), 2.42-2.29 (m, 1H), 2.21-1.62 (m, 2H), 1.54-1.17 (m, 5H), |
| | pyrazolo[4,3-b]pyridin-2- | | 0.95-0.90 (m, 3H), 0.66-0.58 (m, 3H) ppm. MS: M/e 451 (M + 1)⁺. |
| | yl)acetonitrile | | |
| A68 | 2-(7-((2S,5R)-2,5-diethyl-4-(1- | 6 mg | ¹H NMR (400 MHz, CD₃OD δ 8.55 (d, J = 14.8 Hz, 1H), 8.44 (s, 1H), 7.92 (s, 2H), |
| | (pyridine-3-yl)ethyl)piperazin-1-yl)- | | 7.43 (s, 1H), 5.56 (s, 1H), 5.47 (s, 2H), 3.81-3.71 (m, 1H), 3.51 (d, J = 14.2 Hz, 1H), |
| | 4-methyl-5-oxo-4,5-dihydro-2H- | | 3.43 (s, 3H), 3.21-2.98 (m, 1H), 2.93-2.68 (m, 1H), 2.41-2.26 (m, 1H), 2.16-1.77 |
| | pyrazolo[4,3-b]pyridin-2- | | (m, 2H), 1.82-1.56 (m, 3H), 1.43-1.26 (m, 4H), 1.03-0.95 (m, 3H), 0.72-0.61 (m, 3H) |
| | yl)acetonitrile | | ppm. MS: M/e 434(M + 1)⁺. |
| A70 | 2-(7-((2S,5R)-2,5-diethyl-4-(4- | 1 mg, 19% | ¹H NMR (400 MHz, CD₃OD) δ 7.93 (s, 1H), 7.40-7.24 (m, 5H), 5.57 (s, 1H), 5.47 (s, |
| | fluorobenzyl)piperazin-1-yl)-4- | | 2H), 4.60-4.29 (m, 2H), 3.64-3.62 (m, 2H), 3.51-3.49 (m, 1H), 3.48 (d, J = 13.0 Hz, |
| | methyl-5-oxo-4,5-dihydro-2H- | | 1H), 3.43 (s, 3H), 2.32-2.89(m, 2H), 1.39-1.11(m, 9H) ppm. MS: M/e 437 (M + 1)⁺. |
| | pyrazolo[4,3-b]pyridin-2- | | |
| | yl)acetonitrile | | |
| A72 | 2-(7-((2S,5R)-4-(1-(4-cyclopropyl- | A72a | A72a: ¹H NMR (400 MHz, CDCl₃) δ 8.57-8.53 (m, 0.2H) 7.42-7.26 (m, 1.5H), 6.84- |
| | 2-fluorophenyl)ethyl)-2,5- | (3 mg) | 6.66 (m, 2.3H), 6.05-5.59 (m, 1H), 5.15-5.11 (m, 2H), 4.60-4.37 (m, 1H), 3.96-3.92 |
| | diethylpiperazin-1-yl)-4-methyl-5- | | (m, 1H), 3.55-3.51 (m, 1H), 3.41 (s, 3H), 3.15-2.65 (m, 3H), 2.63-1.59 (m, 4H), 1.42- |
| | oxo-4,5-dihydro-2H-pyrazolo[4,3- | | 1.25 (m, 5H), 1.23-0.73 (m, 5H), 0.71-0.62 (m, 5H) ppm. MS: M/e 491 (M + 1)⁺. |
| | b]pyridin-2-yl)acetonitrile | A72b | A72b: ¹H NMR (400 MHz, CDCl₃) δ 8.84-8.80 (m, 0.3H), 7.42-7.27 (m, 1.4H), 7.04- |
| | | (5 mg) | 6.60 (m, 2.4H), 5.63-5.59 (m, 1H), 5.12 (m, 2H), 4.52-4.09 (m, 2H), 3.72-3.52 (m, 1H), |
| | | | 3.42 (s, 3H), 3.15-2.85 (m, 3H), 2.40-1.58 (m, 4H), 1.42-1.26 (m, 5H), 1.13-0.71 (m, |
| | | | 5H), 0.90-0.56 (m, 5H) ppm. MS: M/e 491 (M + 1)⁺. |
| A73 | 2-(7-((2S,5R)-2,5-diethyl-4-(1-(2- | 15 mg, 15% | ¹H NMR (400 MHz, CDCl₃) δ 7.42-7.38 (m, 1H), 7.23-7.17 (m, 2H), 7.01-7.00 (m, |
| | fluoro-4- | | 2H), 5.65-5.59 (m, 1H), 5.15-5.11 (m, 2H), 4.46-3.97 (m, 2H), 3.53-3.48 (m, 2H), 3.41 |
| | (trifluoromethyl)phenyl)ethyl)piperazin- | | (s, 3H), 3.16-2.92 (m, 3H), 2.57-2.54 (m, 2H), 2.07-1.78 (m, 4H), 1.53-1.48 (m, 1H), |
| | 1-yl)-4-methyl-5-oxo-4,5- | | 1.26-1.06 (m, 3.5H), 1.01-0.95 (m, 4H), 0.72-0.59 (m, 4.5H) ppm. MS: M/e 473 |
| | dihydro-2H-pyrazolo[4,3-b]pyridin- | | (M + 1)⁺. |
| | 2-yl)acetonitrile | | |
| A74 | 2-(7-((2S,5R)-2,5-diethyl-4-(1-(5- | A74a | A74a (the earlier peak): ¹H NMR (400 MHz, CD₃OD) δ 8.36 (s, 1H), 7.91 (s, 1H), 7.63 |
| | fluoropyridin-2-yl)ethyl)piperazin- | (26 mg) | (d, J = 6.0 Hz, 2H), 5.54 (s, 1H), 5.45 (s, 2H), 4.55 (br s, 1H), 3.95 (q, J = 6.5 Hz, 1H), |
| | 1-yl)-4-methyl-5-oxo-4,5-dihydro- | | 3.42 (s, 3H), 3.32 (br s, 1H), 2.96-2.88 (m, 2H), 2.32 (br s, 1H), 2.19-1.74 (m, 3H), |
| | 2H-pyrazolo[4,3-b]pyridin-2- | | 1.55 (dd, J = 14.5, 7.2 Hz, 2H), 1.35 (d, J = 6.6 Hz, 3H), 0.93 (t, J = 7.5 Hz, 3H), 0.71 |
| | yl)acetonitrile | | (t, J = 7.4 Hz, 3H) ppm. MS: M/e 452(M + 1)⁺. |
| | | A74b | A74b (the later peak): ¹H NMR (400 MHz, CD₃OD) δ 8.36 (brs, 1H), 7.93 (s, 1H), 7.72- |
| | | (23 mg) | 7.58 (m, 2H), 5.56 (s, 1H), 5.47 (s, 2H), 3.78-3.76 (m, 1H), 3.48-3.46 (m, 1H), |
| | | | 3.43 (s, 3H), 3.11 (br s, 1H), 2.75-2.73 (m, 2H), 2.24-2.21 (m, 1H), 1.94-1.93 (m, |
| | | | 1H), 1.69-1.51 (m, 4H), 1.32 (d, J = 6.6 Hz, 3H), 1.00 (t, J = 7.2 Hz, 3H), 0.63 (t, J = |
| | | | 7.3 Hz, 3H) ppm. MS: M/e 452(M + 1)⁺. |
| A75 | 2-(7-((2S,5R)-4-(2,4- | 85 mg, 32% | ¹H NMR (400 MHz, CDCl₃) δ 7.43-7.40 (m, 2H), 6.86-6.79 (m, 2H), 5.61 (s, 1H), 5.20- |
| | difluorobenzyl)-2,5- | | 5.11 (m, 2H), 4.38-4.34 (m, 2H), 3.64 (s, 2H), 3.37 (s, 3H), 3.34-3.30 (m, 1H), 3.18 (s, |
| | diethylpiperazin-1-yl)-4-methyl-5- | | 1H), 2.86-2.82 (m, 1H), 2.67-2.63 (m, 1H), 2.48-2.45 (m, 1H), 1.92-1.88 (m, 1H), 1.67- |
| | oxo-4,5-dihydro-2H-pyrazolo[4,3- | | 1.63 (m, 2H), 0.91 (s, 3H), 0.73 (s, 3H) ppm. MS: M/e 455 (M + 1)⁺. |
| | b]pyridine-2-yl)acetonitrile | | |
| A76 | 2-(7-((2S,5R)-4-(1-(4-cyclopropyl- | 11 mg, 20% | ¹H NMR (400 MHz, CDCl₃) δ 7.38-7.15 (m, 2H), 6.86-6.66 (m, 2H), 5.58 (s, 1H), 5.13 |
| | 2-fluorophenyl)propyl)-2,5- | | (m, 2H), 4.36-3.80 (m, 2H), 3.50-3.41 (m, 4H), 3.18 (s, 1H), 3.02-2.90 (m, 1H), 2.61- |
| | diethylpiperazin-1-yl)-4-methyl-5- | | 2.50(m, 1H), 2.35-2.32(m, 3H), 2.08-1.86 (m, 3H), 1.70 (s, 3H), 1.46-0.96 (m, 6H), |
| | oxo-4,5-dihydro-2H-pyrazolo[4,3- | | 0.69-0.60 (m, 8H) ppm. MS: M/e 505 (M + 1)⁺. |
| | b]pyridine-2-yl)acetonitrile | | |
| A77 | 2-(7-((2S,5R)-2,5-diethyl-4-(4- | 4 mg, 26% | ¹HNMR (400 MHz, CD₃OD) δ 7.93 (s, 1H), 7.49 (d, J = 7.6 Hz, 2H), 7.23 (d, J = 6.8 |
| | (trifluoromethoxy)benzyl)piperazin- | | Hz, 2H), 5.57 (s, 1H), 5.48 (s, 2H), 4.89-4.18 (m, 2H), 3.80-3.60 (m, 2H), 3.54- |
| | 1-yl)-4-methyl-5-oxo-4,5-dihydro- | | 3.46 (m, 1H), 3.44 (s, 3H), 2.91-2.65 (m, 2H), 2.54-2.44 (m, 1H), 2.05-1.89 (m, |
| | 2H-pyrazolo[4,3-b]pyridin-2- | | 1H), 1.85-1.70 (m, 1H), 1.66-1.54 (m, 2H), 1.00-0.90 (m, 3H), 0.81-0.72 (m, |
| | yl)acetonitrile | | 3H) ppm. MS: M/e 503 (M + 1)⁺. |
| A78 | 2-(7-((2S,5R)-2,5-diethyl-4-(1-(3- | 13 mg, 20% | ¹H NMR (400 MHz, CD₃OD) δ 7.92 (s, 1H), 7.68-7.56 (m, 2H), 7.17-7.11 (m, 1H), |
| | methoxy-4- | | 5.55(s, 1H), 5.47 (s, 2H), 3.90 (s, 3H), 3.78-3.47 (m, 2H), 3.43 (s, 3H), 3.31-3.14 (m, |
| | (trifluoromethyl)phenyl)ethyl)piperazin- | | 2H), 3.02-2.50 (m, 2H), 2.42-2.25 (m, 1H), 2.25-1.52 (m, 4H), 1.34-1.30 (m, 3H), 1.05- |
| | 1-yl)-4-methyl-5-oxo-4,5- | | 0.95 (m, 3H), 0.76-0.62 (m, 3H) ppm. MS: M/e 531 (M + 1)⁺. |
| | dihydro-2H-pyrazolo[4,3-b]pyridin- | | |
| | 2-yl)acetonitrile | | |
| A80 | 2-(7-((2S,5R)-2,5-diethyl-4-(1-(2- | 0.78 mg | ¹H NMR (400 MHz, CD₃OD) δ 7.92 (s, 1H), 7.76-7.72 (m, 1H), 7.45-7.20 (m, 3H), |
| | (trifluoromethoxy)phenyl)ethyl)piperazin- | | 5.58-5.41 (m, 3H), 4.13 (d, J = 53.3 Hz, 1H), 3.55-3.37 (m, 4H), 3.05-2.62 (m, |
| | 1-yl)-4-methyl-5-oxo-4,5- | | 4H), 2.10-2.04 (m, 2H), 1.31 (d, J = 12.1 Hz, 5H), 1.02-0.98 (m, 4H), 0.68-0.64 (m, |
| | dihydro-2H-pyrazolo[4,3-b]pyridin- | | 3H) ppm. MS: M/e 517(M + 1)⁺. |
| | 2-yl)acetonitrile | | |
| A81 | 2-(7-((2S,5R)-2,5-diethyl-4-(1-(6- | 23 mg, 26% | ¹H NMR (400 MHz, CD₃OD) δ 8.05-8.01 (m, 1H), 7.93 (s, 1H), 7.72 (m, 1H), 6.76- |
| | isopropoxypyridin-3- | | 6.71 (m, 1H), 5.55(s, 1H), 5.47 (s, 2H), 5.20-5.18 (m, 1H), 3.75-3.49 (m, 2H), 3.43 (s, |
| | yl)ethyl)piperazin-1-yl)-4-methyl-5- | | 3H), 3.26-3.13 (m, 1H), 3.00-2.63 (m, 2H), 2.36 (m, 1H), 2.08-1.80 (m, 2H), 1.75-1.53 |
| | oxo-4,5-dihydro-2H-pyrazolo[4,3- | | (m, 3H), 1.33 (s, 9H), 1.05-0.95 (m, 3H), 0.73-0.64 (m, 3H) ppm. MS: M/e 492 (M + 1)⁺. |
| | b]pyridin-2-yl)acetonitrile | | |
| A82 | 2-(7-((2S,5R)-2,5-diethyl-4-(1-(4- | 5 mg, 12% | ¹H NMR (400 MHz, CD₃OD) δ 7.93 (s, 1H), 7.25-6.81 (m, 3H), 5.56 (s, 1H), 5.47 (s, |
| | fluoro-3- | | 2H), 3.87 (s, 3H), 3.75-3.30 (m, 4H), 3.25-2.97 (m, 1H), 2.96-2.51 (m, 2H), 2.50- |
| | methoxyphenyl)ethyl)piperazin-1- | | 2.25 (m, 1H), 2.24-1.57 (m, 2H), 1.56-1.41 (m, 3H), 1.38-1.20 (m, 4H), 1.20- |
| | yl)-4-methyl-5-oxo-4,5-dihydro-2H- | | 0.80 (m, 3H), 0.75-0.51 (m, 3H) ppm. MS: M/e 481(M + 1)⁺. |
| | pyrazolo[4,3-b]pyridin-2- | | |
| | yl)acetonitrile | | |
| A83 | 2-(7-((2S,5R)-4-(1-(2,6- | 2 mg, 3% for two | ¹H NMR (400 MHz, CD₃OD) δ 7.92 (s, 1H), 7.30 (s, 1H), 7.04-6.83 (m, 2H), 5.53 (s, |
| | difluorophenyl)ethyl)-2,5- | steps | 1H), 5.47 (s, 3H), 4.27-4.23 (m, 1H), 3.43 (s, 4H), 3.13 (s, 1H), 2.92 (d, J = 18.5 Hz, |
| | diethylpiperazin-1-yl)-4-methyl-5- | | 1H), 2.78 (d, J = 11.5 Hz, 1H), 2.39 (d, J = 9.5 Hz, 1H), 1.95 (s, 1H), 1.82-1.45 (m, |
| | oxo-4,5-dihydro-2H-pyrazolo[4,3- | | 6H), 0.98 (d, J = 25.9 Hz, 3H), 0.69-0.65 (m, 3H) ppm. MS: M/e 469(M + 1)⁺. |
| | b]pyridin-2-yl)acetonitrile | | |
| A85 | 2-(7-((2S,5R)-2,5-diethyl-4-(1-(4- | 8 mg, 24% | ¹H NMR (400 MHz, CD₃OD) δ 7.92 (s, 1H), 7.56-7.48 (m, 1H), 6.76-6.68 (m, 2H), |
| | fluoro-2-(2- | | 5.54 (s, 1H), 5.47 (s, 2H), 4.25-4.01 (m, 3H), 3.78-3.75 (m, 2H), 3.49-3.43 (m, |
| | methoxyethoxy)phenyl)ethyl)piperazin- | | 6H), 2.94-2.89 (m, 1H), 2.49-2.30 (m, 1H), 2.15-1.80 (m, 2H), 1.70-1.54 (m, |
| | 1-yl)-4-methyl-5-oxo-4,5- | | 3H), 1.29-1.23 (m, 5H), 1.11-0.84 (m, 5H), 0.72-0.67 (m, 2H) ppm. MS: M/e 525 |
| | dihydro-2H-pyrazolo[4,3-b]pyridin- | | (M + 1)⁺. |
| | 2-yl)acetonitrile | | |
| A86 | 2-(7-((2S,5R)-2,5-diethyl-4-(1- | 2 mg, 2% | ¹H NMR (400 MHz, CD₃OD) δ 8.49 (s, 2H), 7.93 (s, 1H), 7.50 (s, 2H), 5.56 (s, 1H), |
| | (pyridin-4-yl)ethyl)piperazin-1-yl)- | | 5.47 (s, 2H), 3.84 (s, 0.5H), 3.69 (s, 0.5H), 3.44 (s, 4H), 3.13 (s, 2H), 2.98 (s, 1H), 2.91 |
| | 4-methyl-5-oxo-4,5-dihydro-2H- | | (s, 1H), 2.74-2.68 (m, 1H), 2.04-1.93 (m, 1H), 1.85 (s, 1H), 1.69 (s, 2H), 1.56 (s, |
| | pyrazolo[4,3-b]pyridin-2- | | 3H), 0.99 (d, J = 26.4 Hz, 3H), 0.70 (d, J = 30.5 Hz, 3H) ppm. MS: M/e 434 (M + 1)⁺. |
| | yl)acetonitrile | | |
| A87 | 2-(7-((2S,5R)-4-(1-(3,5- | 1 mg, 12% | ¹H NMR (400 MHz, CD₃OD) δ 8.35 (d, J = 11.3 Hz, 2H), 7.93 (s, 1H), 5.55 (s, 1H), |
| | difluoropyridin-4-yl)ethyl)-2,5- | | 5.47 (s, 2H), 4.40 (s, 0.5H), 4.25 (s, 0.5H), 3.43 (s, 4H), 3.13 (s, 1H), 3.02-2.94 (m, |
| | diethylpiperazin-1-yl)-4-methyl-5- | | 1H), 2.88 (s, 2H), 2.35 (s, 1H), 1.65 (s, 2H), 1.53 (s, 3H), 1.29 (s, 2H), 0.98 (d, J = 27.4 |
| | oxo-4,5-dihydro-2H-pyrazolo[4,3- | | Hz, 3H), 0.81 (s, 1.5H), 0.60 (s, 1.5H) ppm. MS: M/e 470 (M + 1)⁺. |
| | b]pyridin-2-yl)acetonitrile | | |
| A88 | 2-(7-((2S,5R)-2,5-diethyl-4-(1- | 3 mg, 12% | ¹H NMR (400 MHz, CD₃OD) δ 8.82 (d, J = 24.5 Hz, 1H), 8.55 (s, 1H), 8.51 (s, 1H), |
| | (pyrazin-2-yl)ethyl)piperazin-1-yl)- | | 7.93 (s, 1H), 5.56 (s, 1H), 5.47 (s, 2H), 4.05 (s, 0.5H), 3.85 (s, 0.5H), 3.43 (s, 4H), 3.19- |
| | 4-methyl-5-oxo-4,5-dihydro-2H- | | 3.12 (m, 1H), 2.96 (s, 2H), 2.79 (s, 1H), 2.40 (s, 1H), 1.71 (s, 2H), 1.60 (s, 2H), 1.43 |
| | pyrazolo[4,3-b]pyridin-2- | | (d, J = 16.2 Hz, 3H), 1.03 (s, 1.5H), 0.93 (s, 1.5H), 0.77 (s, 1.5H), 0.66 (s, 1.5H) ppm. |
| | yl)acetonitrile | | MS: M/e 435 (M + 1)⁺. |
| A89 | 2-(7-((2S,5R)-2,5-diethyl-4-(1- | 7 mg, 13% for two | ¹H NMR (400 MHz, DMSO-d6) δ 7.98 (s, 1H), 7.30 (m, 5H), 5.61 (s, 2H), 5.39 (s, 1H), |
| | phenylethyl)piperazin-1-yl)-4- | steps | 3.62 (d, J = 52.5 Hz, 1H), 3.34 (s, 2H), 3.27 (s, 3H), 3.17-2.62 (m, 3H), 2.38-2.18 |
| | methyl-5-oxo-4,5-dihydro-2H- | | (m, 1H), 1.97-1.93 (m, 1H), 1.52 (d, J = 62.4 Hz, 3H), 1.26 (d, J = 13.5 Hz, 3H), 0.91 |
| | pyrazolo[4,3-b]pyridin-2- | | (d, J = 33.4 Hz, 3H), 0.56 (d, J = 29.3 Hz, 3H) ppm. MS: M/e 433 (M + 1)⁺. |
| | yl)acetonitrile | | |
| A90 | 2-(7-((2S,5R)-2,5-diethyl-4-(1- | 5 mg, 8% | ¹H NMR (400 MHz, CD₃OD) δ 7.84 (t, J = 5.3 Hz, 1H), 7.31 (d, J = 1.9 Hz, 1H), 7.24 |
| | (pyridin-2-yl)ethyl)piperazin-1-yl)- | | (dd, J = 12.9, 6.3 Hz, 1H), 7.03 (dd, J = 19.6, 7.9 Hz, 1H), 6.76-6.62 (m, 1H), 4.94 (s, |
| | 4-methyl-5-oxo-4,5-dihydro-2H- | | 1H), 4.85 (d, J = 3.4 Hz, 2H), 3.35-3.11 (m, 1H), 2.90 (d, J = 13.1 Hz, 1H), 2.82 (s, |
| | pyrazolo[4,3-b]pyridin-2- | | 3H), 2.49-2.45 (m, 1H), 2.34-2.07 (m, 2H), 1.73 (s, 1H), 1.63-1.45 (m, 1H), 1.41- |
| | yl)acetonitrile | | 1.18 (m, 1H), 1.13-0.86 (m, 3H), 0.77-0.70 (m, 3H), 0.42-0.36 (m, 3H), 0.07-0.03 |
| | | | (m, 3H) ppm. MS: M/e 434 (M + 1)⁺. |
| A91 | 2-(7-((2S,5R)-2,5-diethyl-4-(1-(3- | 1 mg | ¹H NMR (400 MHz, CD₃OD) δ 8.25-8.16 (m, 2H), 7.92 (s, 1H), 6.71-7.58 (m, 1H), |
| | methoxypyridin-4- | | 5.55 (s, 1H), 5.46-5.33 (m, 2H), 4.35-4.16 (m, 1H), 3.96 (s, 3H), 3.50 (m, 1H), 3.43 (s, |
| | yl)ethyl)piperazin-1-yl)-4-methyl-5- | | 3H), 2.95-2.67 (m, 3H), 2.29-2.02 (m, 2H), 1.53-1.25 (m, 4H), 1.02-0.70 (m, 6H), 0.74- |
| | oxo-4,5-dihydro-2H-pyrazolo[4,3- | | 0.69 (m, 3H) ppm. MS: M/e 464 (M + 1)⁺. |
| | b]pyridin-2-yl)acetonitrile | | |
| A92 | 2-(7-((2S,5R)-2,5-diethyl-4-(1-(3- | A92a | A92a: ¹H NMR (400 MHz, CD₃OD) δ 8.14 (s, 1H), 7.92 (s, 1H), 7.42-7.30 (m, 2H), |
| | methoxypyridin-2- | (3 mg, 6%) | 5.51 (s, 1H), 5.45 (s, 2H), 4.48-4.35 (m, 1H), 3.87 (s, 3H), 3.52-3.45 (m, 1H), 3.41 |
| | yl)ethyl)piperazin-1-yl)-4-methyl-5- | | (s, 3H), 2.85-2.68 (m, 4H), 2.32-2.18 (m 1H), 1.96-1.90 (m, 1H), 1.65-1.48 (m, |
| | oxo-4,5-dihydro-2H-pyrazolo[4,3- | | 3H), 1.34-1.27 (m, 3H), 1.01-0.80 (m, 3H), 0.70-0.60 (m, 3H) ppm. MS: M/e 464 |
| | b]pyridin-2-yl)acetonitrile | | (M + 1)⁺. |
| | | A92b | A92b: ¹H NMR (400 MHz, CD₃OD) δ 8.16 (d, J = 3.7 Hz, 1H), 7.92 (s, 1H), 7.48- |
| | | (2 mg, 4%) | 7.44 (m, 1H), 7.34-7.31 (m, 1H), 5.53 (s, 1H), 5.46 (s, 2H), 4.59-4.57 (m, 1H), 4.45 |
| | | | (br s, 1H), 3.90 (s, 3H), 3.43 (s, 3H), 3.11-3.08 (m, 1H), 2.97-2.93 (m 1H), 2.72 (br |
| | | | s, 1H), 2.52 (br s, 1H), 2.21-2.01 (m, 1H), 1.93-1.90 (m, 1H), 1.77-1.66 (m, 1H), |
| | | | 1.60-1.54 (m, 2H), 1.44 (d, J = 6.7 Hz, 3H), 0.90 (t, J = 7.5 Hz, 3H), 0.75 (t, J = 7.3 |
| | | | Hz, 3H) ppm. MS: M/e 464 (M + 1)⁺. |
| A93 | 2-(7-((2S,5R)-2,5-diethyl-4-(1-(3- | 11 mg | ¹H NMR (400 MHz, CD₃OD) δ 8.54-8.40 (m, 1H), 7.92 (s, 1H), 7.80-7.91 (m, 1H), |
| | (methoxymethyl)pyridin-2- | | 7.37-7.29 (m, 1H), 5.55 (s, 1H), 5.47 (d, J = 3.4 Hz, 2H), 4.84-4.60 (m, 2H), 4.22- |
| | yl)ethyl)piperazin-1-yl)-4-methyl-5- | | 4.05 (m, 1H), 3.65-3.47 (m, 1H), 3.46 (s, 2H), 3.43 (s, 4H), 3.41-3.32 (m, 2H), 3.17- |
| | oxo-4,5-dihydro-2H-pyrazolo[4,3- | | 2.97 (m, 1H), 2.93-2.72 (m, 2H), 2.07-1.81 (m, 1H), 1.81-1.63 (m, 1H), 1.63-1.50 (m, |
| | b]pyridin-2-yl)acetonitrile | | 2H), 1.44 (d, J = 6.6 Hz, 2H), 1.38 (d, J = 6.3 Hz, 1H), 1.03 (t, J = 7.3 Hz, 1H), 0.87 (t, |
| | | | J = 7.1 Hz, 2H), 0.68 (t, J = 7.1 Hz, 2H), 0.55 (t, J = 7.3 Hz, 1H) ppm. MS: M/e 478 |
| | | | (M + 1)⁺. |
| A94 | 2-(7-((2S,5R)-2,5-diethyl-4-(1-(4- | 2 mg, 7% | ¹H NMR (400 MHz, CD₃OD) δ 7.92 (s, 1H), 6.38-6.30 (m, 2H), 5.52 (s, 1H), 5.47 (s, |
| | fluoro-2,6- | | 2H), 4.37-4.30 (m, 1H), 3.89-3.82 (m, 7H), 3.48-3.43 (m, 4H), 2.90-2.72 (m, |
| | dimethoxyphenyl)ethyl)piperazin-1- | | 4H), 2.45-2.36 (m 1H), 1.99-1.92 (m, 1H), 1.52-1.42 (m, 5H), 1.01-0.95 (m, 3H), |
| | yl)-4-methyl-5-oxo-4,5-dihydro-2H- | | 0.75-0.60 (m, 3H) ppm. MS: M/e 511 (M + 1)⁺. |
| | pyrazolo[4,3-b]pyridin-2- | | |
| | yl)acetonitrile | | |
| A104 | 2-(7-((2S,5R)-2,5-diethyl-4-(1-(1- | 1 mg, 5% | ¹H NMR (400 MHz, CD₃OD) δ 8.43 (br s, 1H, HCOOH), 7.94 (s, 1H), 7.78-7.72 (m, |
| | methyl-2-oxo-1,2-dihydropyridin-3- | | 1H), 7.60 (d, J = 6.6 Hz, 1H), 6.44 (t, J = 6.9 Hz, 1H), 5.57 (s, 1H), 5.48 (s, 2H), 4.30- |
| | yl)ethyl)piperazin-1-yl)-4-methyl-5- | | 4.28 (m, 0.5H), 4.10-4.08 (s, 0.5H), 3.60 (s, 3H), 3.55-3.50 (m, 0.5H), 3.44 (s, |
| | oxo-4,5-dihydro-2H-pyrazolo[4,3- | | 3H), 3.25-3.18 (m, 0.5H), 3.00 (br s, 1H), 2.75-2.60 (m, 2H), 2.03-1.49 (m, 5H), |
| | b]pyridin-2-yl)acetonitrile | | 1.37-1.21 (m, 5H), 1.05-0.92 (m, 3H), 0.86-0.71 (m, 3H) ppm. MS: M/e 464 |
| | | | (M + 1)⁺. |
| A105 | 2-(7-((2S,5R)-2,5-diethyl-4-(1-(2- | 37 mg, 48% | ¹H NMR (400 MHz, CD₃OD) δ 8.04-7.84 (m, 3H), 6.99-6.96 (m, 1H), 5.57 (s, 1H), |
| | methoxypyridin-3- | | 5.47 (s, 2H), 4.23-4.21 (m, 0.5H), 4.02-3.98 (s, 0.5H), 3.94 (s, 3H), 3.48-3.44 (m, |
| | yl)ethyl)piperazin-1-yl)-4-methyl-5- | | 1H), 3.43 (s, 3H), 3.11-3.08 (m, 1H), 2.96-2.91 (m, 1H), 2.78-2.69 (m, 1H), 2.45- |
| | oxo-4,5-dihydro-2H-pyrazolo[4,3- | | 2.25 (m, 1H), 2.03-1.46 (m, 5H), 1.28-1.22 (m, 3H), 1.07-0.82 (m, 3H), 0.74- |
| | b]pyridin-2-yl)acetonitrile | | 0.67 (m, 3H) ppm. MS: M/e 464 (M + 1)⁺. |
| A106 | 2-(7-((2S,5R)-2,5-diethyl-4-(1-(2- | A106a(0.1 | A106a (the earlier peak): ¹H NMR (400 MHz, CD₃OD) δ 7.93 (s, 1H), 7.57 (d, J = 7.2 |
| | methoxyphenyl)ethyl)piperazin-1- | 7 mg) | Hz, 1H), 7.21 (t, J = 7.6 Hz, 1H), 6.94 (t, J = 7.2 Hz, 2H), 5.54 (s, 1H), 5.47 (s, 2H), |
| | yl)-4-methyl-5-oxo-4,5-dihydro-2H- | | 4.20 (s, 1H), 3.83 (s, 3H), 3.50 (d, J = 14.4 Hz, 1H), 3.43 (s, 3H), 3.19-3.09 (m, 1H), |
| | pyrazolo[4,3-b]pyridin-2- | | 2.93-2.73 (m, 1H), 2.69-2.40 (m, 2H), 2.08-1.96 (m, 1H), 1.72-1.54 (m, 4H), 1.25 |
| | yl)acetonitrile | | (d, J = 6.0 Hz, 3H), 1.02 (t, J = 7.2 Hz, 3H), 0.67 (t, J = 7.2 Hz, 3H) ppm. MS: M/e 463 |
| | | | (M + 1)⁺. |
| | | A106b(0.3 | A106b (the later peak, FA salt): ¹H NMR (400 MHz, CD₃OD) δ 8.47 (s, 2H, HCOOH), |
| | | 2 mg) | 7.92 (s, 1H), 7.48 (d, J = 6.8 Hz, 1H), 7.24 (t, J = 7.6 Hz, 1H), 7.01-6.95 (m, 2H), 5.54 |
| | | | (s, 1H), 5.46 (s, 2H), 4.44-4.34 (m, 1H), 3.83 (s, 3H), 3.61 (d, J = 3.6 Hz, 1H), 3.43 |
| | | | (s, 3H), 2.96 (s, 1H), 2.50 (d, J = 13.2 Hz, 1H), 2.11-1.97 (m, 1H), 1.88-1.75 (m, |
| | | | 1H), 1.66-1.43 (m, 3H), 1.31 (d, J = 6.4 Hz, 3H), 0.96 (t, J = 7.2 Hz, 3H), 0.71 (t, J = |
| | | | 7.2 Hz, 3H) ppm. MS: M/e 463 (M + 1)⁺. |
| A107 | 2-(7-((2S,5R)-2,5-diethyl-4-(1-(2- | 30 mg | ¹H NMR (400 MHz, DMSO-d6) δ 7.98 (s, 1H), 7.61-7.54 (m, 1H), 7.36-7.27 (m, 2H), |
| | (methoxymethyl)phenyl)ethyl)piperazin- | | 7.21 (dd, J = 14.2, 7.4 Hz, 1H), 5.62 (s, 2H), 5.40 (s, 1H), 4.56-4.44 (m, 2H), 4.05-3.80 |
| | 1-yl)-4-methyl-5-oxo-4,5- | | (m, 1H), 3.42-3.30 (m, 1.5H), 3.29 (d, J = 3.0 Hz, 3H), 3.27 (s, 3H), 3.15-3.02 (m, 1H), |
| | dihydro-2H-pyrazolo[4,3-b]pyridin- | | 2.95 (d, J = 11.5 Hz, 0.5H), 2.76 (s, 0.5H), 2.58-2.50 (m, 1.5H), 2.32 (d, J = 8 Hz, |
| | 2-yl)acetonitrile | | 0.5H), 2.14 (d, J = 12.0 Hz, 0.5H), 2.10-1.79 (m, 1H), 1.69-1.36 (m, 3H), 1.22 (dd, |
| | | | J = 12.4, 6.3 Hz, 3H), 1.00-0.80 (m, 3H), 0.65-0.45 (m, 3H) ppm. MS: M/e 477 (M + 1)⁺. |
| A108 | 3-(1-((2R,5S)-4-(2-(cyanomethyl)- | 9 mg | ¹H NMR (400 MHz, DMSO-d6) δ 8.02-7.84 (m, 3H), 7.75 (t, J = 7.4 Hz, 1H), 7.53 |
| | 4-methyl-5-oxo-4,5-dihydro-2H- | | (t, J = 7.8 Hz, 1H), 7.45-7.30 (m, 2H), 5.62 (s, 2H), 5.39 (s, 1H), 3.62 (q, J = 6.4 Hz, |
| | pyrazolo[4,3-b]pyridin-7-yl)-2,5- | | 1H), 3.41-3.34 (m, 1.5H), 3.33-3.30 (m, 0.5H), 3.27 (s, 3H), 3.14-3.02 (m, 1H), 3.00- |
| | diethylpiperazin-1- | | 2.88 (m, 0.5H), 2.81-2.63 (m, 0.5H), 2.58-2.53 (m, 0.5H), 2.21 (d, J = 12 Hz, 0.5H), |
| | yl)ethyl)benzamide | | 2.10-1.80 (m, 1H), 1.72-1.38 (m, 3H), 1.31-1.21 (m, 4H), 1.00-0.80 (m, 3H), 0.65- |
| | | | 0.25 (m, 3H) ppm. MS: M/e 476 (M + 1)⁺. |
| A109 | N-(3-(1-((2R,5S)-4-(2- | 4 mg, FA salt | ¹H NMR (400 MHz, DMSO-d6) δ 9.91 (d, J = 9.9 Hz, 1H), 8.38 (s, 1H, HCOOH), 7.98 |
| | (cyanomethyl)-4-methyl-5-oxo-4,5- | | (s, 1H), 7.65-7.44 (m, 2H), 7.26-7.21 (m, 1H), 7.01 (dd, J = 13.5, 7.8 Hz, 1H), 5.62 |
| | dihydro-2H-pyrazolo[4,3-b]pyridin- | | (s, 2H), 5.39 (d, J = 9.1 Hz, 1H), 3.64 (q, J = 6.3 Hz, 0.5H), 3.50 (q, J = 6.1 Hz, 0.5H), |
| | 7-yl)-2,5-diethylpiperazin-1- | | 3.34 (s, 2H), 3.27 (s, 3H), 3.09 (d, J = 9.0 Hz, 0.5H), 3.03 (d, J = 9.0 Hz, 0.5H), 2.89 |
| | yl)ethyl)phenyl)acetamide | | (d, J = 9.0 Hz, 0.5H), 2.77-2.67 (m, 0.5H), 2.54 (s, 0.5H), 2.49-2.44 (m, 0.5H), 2.40- |
| | | | 2.22 (m, 1H), 2.03 (d, J = 3.6 Hz, 3H), 2.02-1.85 (m, 1H), 1.70-1.36 (m, 3H), 1.24 |
| | | | (dd, J = 11.0, 6.5 Hz, 3H), 1.00-0.82 (m, 3H), 0.66-0.32 (m, 3H) ppm. MS: M/e 490 |
| | | | (M + 1)+ |
| A110 | 2-(7-((2S,5R)-2,5-diethyl-4-(1-(3- | A110a | A110a: ¹H NMR (400 MHz, CD₃OD) δ 7.92 (s, 1H), 7.60 (d, J = 8.1 Hz, 1H), 7.43- |
| | methyl-2- | (6 mg) | 7.31 (m, 2H), 5.56 (s, 1H), 5.46 (s, 2H), 4.89 (s, 1H), 3.82-3.77 (m, 1H), 3.43 (s, 3H), |
| | (trifluoromethyl)phenyl)ethyl)piperazin- | | 3.33-3.31 (m, 2H), 3.02-3.00 (m, 1H), 2.88-2.85 (m, 1H), 2.48 (s, 3H), 2.40 (br s, |
| | 1-yl)-4-methyl-5-oxo-4,5- | | 1H), 2.12-2.08 (m, 1H), 1.81-1.76 (m, 1H), 1.60-1.48 (m, 2H), 1.34 (d, J = 6.4 Hz, |
| | dihydro-2H-pyrazolo[4,3-b]pyridin- | | 3H), 0.96 (t, J = 7.4 Hz, 3H), 0.70 (t, J = 7.4 Hz, 3H) ppm. MS: M/e 515 (M + 1)⁺. |
| | 2-yl)acetonitrile | A110b | 110b: ¹H NMR (400 MHz, CD₃OD) δ 7.93 (s, 1H), 7.57 (d, J = 8.0 Hz, 1H), 7.44 (s, |
| | | (8 mg) | 1H), 7.37 (d, J = 7.8 Hz, 1H), 5.55 (s, 1H), 5.47 (s, 2H), 4.95 (s, 1H), 3.66-3.64 (m, |
| | | | 1H), 3.53-3.49 (m, 1H), 3.43 (s, 3H), 3.37 (s, 1H), 3.16-3.14 (m, 1H), 2.67-2.64 |
| | | | (m, 1H), 2.47 (s, 3H), 2.34-2.32 (m, 1H), 2.03-2.01 (m, 1H), 1.58-1.50 (m, 3H), |
| | | | 1.31 (d, J = 6.5 Hz, 3H), 1.03 (t, J = 7.3 Hz, 3H), 0.63 (t, J = 7.3 Hz, 3H) ppm. MS: |
| | | | M/e 515 (M + 1)⁺. |
| A112 | 2-(7-((2S,5R)-2,5-diethyl-4-(1-(4- | 2 mg, 4% | ¹H NMR (400 MHz, CD₃OD) δ 8.17-8.08 (m, 1H), 7.93 (s, 1H), 7.77-7.68 (m, 1H), |
| | fluoro-2- | | 7.49 (t, J = 8.2 Hz, 1H), 5.56 (s, 1H), 5.47 (s, 2H), 4.83-4.55 (m, 1H), 3.52 (d, J = |
| | (methylsulfonyl)phenyl)ethyl)piperazin- | | 13.3 Hz, 1H), 3.43 (s, 3H), 3.24 (d, J = 3.2 Hz, 3H), 3.11 (d, J = 11.4 Hz, 1H), 3.10- |
| | 1-yl)-4-methyl-5-oxo-4,5- | | 2.75 (m, 2H), 2.37 (s, 1H), 2.25-2.10 (m, 1H), 1.86 (s, 1H), 1.75-1.50 (m, 3H), 1.37- |
| | dihydro-2H-pyrazolo[4,3-b]pyridin- | | 1.33 (m, 3H), 1.10-0.90 (m, 3H), 0.76-0.61 (m, 3H) ppm. MS: M/e 529 (M + 1)⁺. |
| | 2-yl)acetonitrile | | |
| A113 | 2-(7-((2S,5R)-2,5-diethyl-4-(1-(6- | 6 mg, 6% | ¹H NMR (400 MHz, CD₃OD) δ 8.87-8.85 (d, J = 8 Hz, 1H), 7.93 (s, 1H), 7.38 (s, 1H), |
| | fluoro-4-(trifluoromethyl)pyridin-3- | | 5.58 (s, 1H), 5.47 (s, 2H), 4.25-3.52 (m, 2H), 3.43 (s, 3H), 3.30-3.10 (m, 2H), 2.93- |
| | yl)ethyl)piperazin-1-yl)-4-methyl-5- | | 2.32 (m, 2H), 2.21-2.17 (m, 1H), 1.96-1.77 (m, 1H), 1.71-1.64 (m, 2H), 1.52-1.46 (m, |
| | oxo-4,5-dihydro-2H-pyrazolo[4,3- | | 1H), 1.37-1.32 (m, 3H), 1.04-0.99 (m, 3H), 0.77-0.60 (m, 3H) ppm. MS: M/e 520 |
| | b]pyridin-2-yl)acetonitrile | | (M + 1)⁺. |
| A114 | 2-(7-((2S,5R)-2,5-diethyl-4-(1-(5- | 2 mg, 6% | ¹H NMR (400 MHz, CD₃OD) δ 7.93 (s, 1H), 7.82 (d, J = 5.0 Hz, 1H), 7.52 (dd, J = 8.0, |
| | methyl-2- | | 4.8 Hz, 1H), 7.21 (d, J = 7.4 Hz, 1H), 5.57 (s, 1H), 5.47 (s, 2H), 4.17 (s, 0.5H), 4.03 (s, |
| | (trifluoromethyl)phenyl)ethyl)piperazin- | | 0.5H), 3.50 (m, 1H), 3.43 (s, 3H), 3.33 (s, 1H), 3.23-3.06 (m, 1H), 2.90 (s, 2H), 2.65 |
| | 1-yl)-4-methyl-5-oxo-4,5- | | (s, 1H), 2.42 (d, J = 10.2 Hz, 3H), 2.09-1.81 (m, 1H), 1.72 (s, 1H), 1.56 (s, 2H), 1.29 |
| | dihydro-2H-pyrazolo[4,3-b]pyridin- | | (t, J = 6.9 Hz, 3H), 1.05 (s, 3H), 0.70 (s, 1.5H), 0.57 (s, 1.5H) ppm. MS: M/e 515 |
| | 2-yl)acetonitrile | | (M + 1)⁺. |
| A116 | 2-(7-((2S,5R)-4-(1-(2-chloro-4- | 27 mg, 28% | ¹H NMR (400 MHz, CD₃OD)δ 7.93 (d, J = 2.4 Hz, 1H), 7.79-7.64 (m, 1H), 7.18 (dd, |
| | fluorophenyl)ethyl)-2,5- | | J = 11.0, 4.5 Hz, 1H), 7.14-7.08 (m, 1H), 5.56 (s, 1H), 5.47 (d, J = 3.3 Hz, 2H), 4.23 |
| | diethylpiperazin-1-yl)-4-methyl-5- | | (dd, J = 61.9, 6.6 Hz, 1H), 3.49 (d, J = 13.3 Hz, 1H), 3.43 (s, 3H), 3.30-3.25 (m, 2H), |
| | oxo-4,5-dihydro-2H-pyrazolo[4,3- | | 3.19-2.99 (m, 1H), 2.91-2.67 (m, 1H), 2.42-2.23 (m, 1H), 2.06-2.02 (d, J = 54.4 |
| | b]pyridin-2-yl)acetonitrile | | Hz, 1H), 1.84-1.50 (m, 3H), 1.28 (dd, J = 9.4, 6.7 Hz, 3H), 1.04-0.98 (m, 3H), 0.76- |
| | | | 0.71 (m, 3H) ppm. MS: M/e 485 (M + 1)⁺. |
| A117 | 2-(7-((2S,5R)-2,5-diethyl-4-(1-(2- | 0.2 mg, 1% | ¹H NMR (400 MHz, CD₃OD) δ 7.92 (s, 1H), 7.57-7.49 (m, 1H), 7.41 (s, 1H), 7.30 (s, |
| | methyl-6- | | 1H), 5.56 (s, 1H), 5.47 (s, 2H), 4.24 (s, 1H), 3.48 (s, 1H), 3.43 (s, 3H), 3.21-3.15 (m, |
| | (trifluoromethyl)phenyl)ethyl)piperazin- | | 1H), 3.13 (s, 1H), 3.04 (s, 1H), 2.83 (s, 3H), 2.80 (s, 2H), 2.03 (d, J = 5.5 Hz, 2H), 1.60 |
| | 1-yl)-4-methyl-5-oxo-4,5- | | (s, 2H), 1.45 (dd, J = 6.7, 3.8 Hz, 3H), 0.90 (t, J = 6.5 Hz, 3H), 0.54 (s, 3H) ppm. MS: |
| | dihydro-2H-pyrazolo[4,3-b]pyridin- | | M/e 515 (M + 1)⁺. |
| | 2-yl)acetonitrile | | |
| A118 | 2-(7-((2S,5R)-2,5-diethyl-4-(1-(3- | 2 mg, 18% | ¹H NMR (400 MHz, CD3OD)δ 8.83 (d, J = 14.6 Hz, 2H), 8.10 (s, 1H), 8.00 (s, 1H), |
| | (trifluoromethyl)pyridin-4- | | 5.65-5.33 (m, 3H), 4.10 (dd, J = 14.2, 7.1 Hz, 1H), 3.54 (d, J = 6.6 Hz, 1H), 3.47 (s, |
| | yl)ethyl)piperazin-1-yl)-4-methyl-5- | | 3H), 3.13 (s, 1H), 2.96-2.79 (m, 1H), 2.32-2.06 (m, 2H), 1.92 (s, 1H), 1.73 (s, 1H), |
| | oxo-4,5-dihydro-2H-pyrazolo[4,3- | | 1.70-1.52 (m, 3H), 1.24 (t, J = 7.1 Hz, 3H), 1.05 (s, 3H), 0.80-0.62 (m, 3H) ppm. |
| | b]pyridin-2-yl)acetonitrile | | MS: M/e 502 (M + 1)⁺. |
| A120 | 4-(1-((2R,5S)-4-(2-(cyanomethyl)- | separated isomer | A120a: ¹H NMR (400 MHz, CD₃OD) δ 7.92 (s, 1H), 7.86 (d, J = 8.1 Hz, 2H), 7.48 (d, |
| | 4-methyl-5-oxo-4,5-dihydro-2H- | A120a (14 | J = 8.2 Hz, 2H), 5.55 (s, 1H), 5.46 (s, 2H), 3.82 (d, J = 6.3 Hz, 1H), 3.43 (s, 3H), 3.27 |
| | pyrazolo[4,3-b]pyridin-7-yl)-2,5- | mg, 34%) | (s, 2H), 3.03 (d, J = 12.2 Hz, 1H), 2.91-2.84 (m, 1H), 2.41 (s, 1H), 2.19-2.08 (m, |
| | diethylpiperazin-1- | | 1H), 1.90-1.79 (m, 1H), 1.58-1.50 (m, 2H), 1.35 (d, J = 6.4 Hz, 3H), 1.29 (s, 1H), |
| | yl)ethyl)benzamide | | 0.96 (t, J = 7.4 Hz, 3H), 0.69 (t, J = 7.3 Hz, 3H) ppm. MS: M/e 476 (M + 1)⁺. |
| | | separated isomer | A120b: ¹H NMR (400 MHz, CD₃OD) δ 7.93 (s, 1H), 7.84 (d, J = 8.1 Hz, 2H), 7.51 (d, |
| | | A120b (6 | J = 8.2 Hz, 2H), 5.55 (s, 1H), 5.47 (s, 2H), 3.68 (d, J = 6.4 Hz, 1H), 3.51 (d, J = 13.4 |
| | | mg, 15%) | Hz, 1H), 3.43 (s, 3H), 3.15 (d, J = 11.1 Hz, 1H), 2.66 (d, J = 6.8 Hz, 1H), 2.60-2.39 |
| | | | (m, 1H), 2.34 (d, J = 12.1 Hz, 1H), 2.06-1.92 (m, 1H), 1.74-1.63 (m, 2H), 1.59- |
| | | | 1.53 (m, 1H), 1.33 (t, J = 6.9 Hz, 4H), 1.03 (t, J = 7.3 Hz, 3H), 0.63 (t, J = 7.3 Hz, 3H) |
| | | | ppm. MS: M/e 476 (M + 1)⁺. |
| A121 | 2-(7-((2S,5R)-2,5-diethyl-4-(1-(3- | separated isomer | A121a: ¹H NMR (400 MHz, CD₃OD) δ 7.92 (s, 1H), 7.38 (s, 1H), 7.34-7.22 (m, 3H), |
| | (hydroxymethyl)phenyl)ethyl)piperazin- | A121a (4 | 5.55 (s, 1H), 5.46 (s, 2H), 3.76-3.71 (m, 1H), 3.43 (s, 3H), 3.05-2.98 (m, 1H), 2.89- |
| | 1-yl)-4-methyl-5-oxo-4,5- | mg, 14%) | 2.84 (m, 1H), 2.44 (s, 1H), 2.18-2.08 (m, 1H), 1.87-1.77 (m, 1H), 1.57-1.47 (m, |
| | dihydro-2H-pyrazolo[4,3-b]pyridin- | | 2H), 1.41-1.24 (m, 6H), 0.96 (t, J = 7.4 Hz, 3H), 0.68 (t, J = 7.3 Hz, 3H) ppm. MS: |
| | 2-yl)acetonitrile | | M/e 463 (M + 1)+ |
| | | A121b (4 | A121b: ¹H NMR (400 MHz, CD₃OD) δ 7.92 (s, 1H), 7.40 (s, 1H), 7.30 (s, 2H), 7.23 |
| | | mg, 14%) | (d, J = 6.4 Hz, 1H), 5.54 (s, 1H), 5.47 (s, 2H), 4.60 (s, 2H), 3.60 (q, J = 6.6 Hz, 1H), |
| | | | 3.49 (d, J = 13.5 Hz, 1H), 3.43 (s, 3H), 3.14 (d, J = 10.0 Hz, 1H), 2.65-2.56 (m, 1H), |
| | | | 2.39 (d, J = 12.0 Hz, 1H), 2.05-1.95 (m, 1H), 1.70-1.52 (m, 3H), 1.31 (d, J = 6.5 |
| | | | Hz, 5H), 1.02 (t, J = 7.4 Hz, 3H), 0.62 (t, J = 7.3 Hz, 3H) ppm. MS: M/e 463 (M + 1)+ |
| A122 | 2-(7-((2S,5R)-2,5-diethyl-4-(1-(4- | 2 mg, 8% | ¹H NMR (400 MHz, CD₃OD) δ 7.92 (d, J = 2.1 Hz, 1H), 7.49 (dd, J = 10.9, 8.7 Hz, |
| | (trifluoromethoxy)phenyl)ethyl)piperazin- | | 2H), 7.24 (t, J = 9.3 Hz, 2H), 5.55 (d, J = 3.8 Hz, 1H), 5.47 (d, J = 3.2 Hz, 2H), 3.84- |
| | 1-yl)-4-methyl-5-oxo-4,5- | | 3.61 (m, 2H), 3.49 (d, J = 11.4 Hz, 1H), 3.43 (s, 3H), 3.10-3.06 (m, 1H), 2.90-2.61 |
| | dihydro-2H-pyrazolo[4,3-b]pyridin- | | (m, 1H), 2.42-2.29 (m, 1H), 2.17-1.77 (m, 2H), 1.72-1.48 (m, 3H), 1.33 (dd, J = |
| | 2-yl)acetonitrile | | 10.3, 6.5 Hz, 3H), 1.02-0.97 (m, 3H), 0.68-0.64 (m, 3H) ppm. MS: M/e 517(M + 1)⁺. |
| A123 | 2-(7-((2S,5R)-2,5-diethyl-4-(1- | 2 mg, 3% | ¹H NMR (400 MHz, CD₃OD) δ 7.94 (s, 1H), 7.86-7.61 (m, 1H), 6.51-6.48 (m, 1H), |
| | (quinoxalin-6-yl)ethyl)piperazin-1- | | 6.41-6.08 (m, 1H), 5.58 (s, 1H), 5.48 (s, 2H), 4.28-4.15 (m, 1H), 3.53-3.48 (s, |
| | yl)-5-oxo-4-(2,2,2-trifluoroethyl)- | | 1H), 3.44 (s, 3H), 3.25-3.00 (m, 2H), 2.85-2.66 (m, 2H), 2.05-1.98 (m, 1H), 1.68- |
| | 4,5-dihydro-2H-pyrazolo[4,3- | | 1.51 (m, 4H), 1.33-1.24 (m, 4H), 1.04-0.79 (m, 6H) ppm. MS: M/e 450.3 (M + 1)⁺. |
| | b]pyridin-2-yl)acetonitrile | | |
| A125 | 2-(4-methyl-5-oxo-7-(5-(1- | A125a | A125a: ¹H NMR (400 MHz, CD₃OD) δ 8.87 (d, J = 5.9 Hz, 2H), 8.09 (d, J = 8.2 Hz, |
| | (quinoxalin-6-yl)ethyl)-2,5- | (2 mg) | 2H), 8.00 (d, J = 8.8 Hz, 1H), 7.93 (s, 1H), 5.71 (s, 1H), 5.47 (s, 2H), 5.35 (s, 1H), 4.08 |
| | diazabicyclo[2.2.2]octan-2-yl)-4,5- | | (q, J = 6.2 Hz, 1H), 3.85 (s, 1H), 3.43 (s, 3H), 3.22 (d, J = 8.7 Hz, 2H), 3.06 (d, J = 9.9 |
| | dihydro-2H-pyrazolo[4,3-b]pyridin- | | Hz, 2H), 2.18-1.94 (m, 3H), 1.62 (s, 1H), 1.44 (d, J = 6.4 Hz, 3H) ppm. |
| | 2-yl)acetonitrile | A125b | A125b: ¹H NMR (400 MHz, CD₃OD) δ 8.86 (d, J = 3.6 Hz, 2H), 8.12-8.04 (m, 2H), |
| | | (2 mg) | 7.99 (d, J = 8.8 Hz, 1H), 7.93 (s, 1H), 5.74 (s, 1H), 5.47 (s, 2H), 5.33 (s, 1H), 4.07 (q, |
| | | | J = 6.4 Hz, 1H), 3.68 (s, 1H), 3.45 (s, 3H), 3.26 (d, J = 9.4 Hz, 2H), 3.12 (s, 1H), 2.93 |
| | | | (d, J = 10.6 Hz, 1H), 2.25 (s, 1H), 1.97 (s, 2H), 1.66 (d, J = 5.7 Hz, 1H), 1.47 (d, J = |
| | | | 6.4 Hz, 3H) ppm. MS: M/e 455(M + 1)⁺. |
| A128 | 2-(7-((2S,5R)-4-(1-(4- | 20 mg, 41% | ¹HNMR (400 MHz, CD₃OD) δ 7.94-7.90 (m, 1H), 7.56-7.43 (m, 4H), 6.89-6.60 |
| | (difluoromethyl)phenyl)ethyl)-2,5- | | (m, 1H), 5.57-5.52 (m, 1H), 5.50-5.42 (m, 2H), 3.84-3.62 (m, 1H), 3.54-3.46 (m, |
| | diethylpiperazin-1-yl)-4-methyl-5- | | 0.5H), 3.43 (s, 3H), 3.31-3.23 (m, 2.5H), 3.20-3.10 (m, 0.5H), 3.06-2.98 (m, 0.5H), |
| | oxo-4,5-dihydro-2H-pyrazolo[4,3- | | 2.92-2.83 (m, 0.5H), 2.70-2.61 (m, 0.5H), 2.42-2.26 (m, 1H), 2.18-2.06 (m, |
| | b]pyridin-2-yl)acetonitrile | | 0.5H), 2.02-1.75 (m, 1H), 1.75-1.47 (m, 2.5H), 1.38-1.28 (m, 3H), 1.03 (t, J = 7.6 |
| | | | Hz, 1.5H), 0.96 (t, J = 7.6 Hz, 1.5H), 0.69 (t, J = 7.6 Hz, 1.5H), 0.62 (t, J = 7.6 Hz, |
| | | | 1.5H) ppm. MS: M/e 483 (M + 1)⁺. |
| A130 | 2-(7-((2S,5R)-2,5-diethyl-4-(1- | 3 mg, 13% | ¹H NMR (400 MHz, CD₃OD) δ 8.80 (d, J = 4.9 Hz, 2H), 7.92 (s, 1H), 7.41 (t, J = 5.0 |
| | (pyrimidin-2-yl)ethyl)piperazin-1- | | Hz, 1H), 5.53 (s, 1H), 5.46 (s, 2H), 4.54-4.46 (m, 0.5H), 4.42-4.32 (m, 0.5H), 4.10 |
| | yl)-4-methyl-5-oxo-4,5-dihydro-2H- | | (d, J = 6.7 Hz, 1H), 3.43 (s, 3H), 3.39 (s, 1H), 3.04 (dd, J = 12.2, 4.3 Hz, 1H), 2.97- |
| | pyrazolo[4,3-b]pyridin-2- | | 2.91 (m, 1H), 2.79 (s, 1H), 2.47 (s, 1H), 1.95-1.86 (m, 1H), 1.73 (s, 1H), 1.58-1.51 |
| | yl)acetonitrile | | (m, 2H), 1.49 (s, 3H), 0.91 (t, J = 7.4 Hz, 3H), 0.75 (t, J = 7.4 Hz, 3H) ppm. MS: M/e |
| | | | 435 (M + 1)⁺. |
| A131 | 2-(7-((2S,5R)-2,5-diethyl-4-(1- | 2 mg, 10% | ¹H NMR (400 MHz, CD₃OD) δ 9.07 (s, 1H), 8.75 (d, J = 5.5 Hz, 1H), 7.94 (s, 1H), |
| | (pyrimidin-4-yl)ethyl)piperazin-1- | | 7.78 (d, J = 4.8 Hz, 1H), 5.57 (s, 1H), 5.48 (s, 2H), 3.75 (s, 1H), 3.55 (d, J = 10.8 Hz, |
| | yl)-4-methyl-5-oxo-4,5-dihydro-2H- | | 1H), 3.44 (s, 3H), 3.13 (s, 2H), 2.80 (s, 2H), 2.27 (d, J = 13.5 Hz, 1H), 1.78 (d, J = 7.1 |
| | pyrazolo[4,3-b]pyridin-2- | | Hz, 2H), 1.56 (d, J = 36.0 Hz, 2H), 1.37 (d, J = 6.4 Hz, 3H), 1.02 (t, J = 7.4 Hz, 3H), |
| | yl)acetonitrile | | 0.71 (t, J = 7.2 Hz, 3H) ppm. MS: M/e 435 (M + 1)⁺. |
| A132 | 2-(7-((2S,5R)-4-(1-(2,4- | A132a | A132a (the earlier peak): ¹H NMR (400 MHz, CD₃OD) δ 7.92 (s, 1H), 7.56 (dd, J = |
| | difluorophenyl)ethyl)-2,5- | (2 mg) | 15.2, 8.3 Hz, 1H), 6.95 (dt, J = 19.9, 8.8 Hz, 2H), 5.55 (s, 1H), 5.46 (s, 2H), 4.15 (q, J = |
| | diethylpiperazin-1-yl)-4-methyl-5- | | 6.4 Hz, 1H), 3.27-3.24 (m, 1H), 3.43 (s, 3H), 2.98-2.85 (m, 3H), 2.41 (s, 1H), 2.11- |
| | oxo-4,5-dihydro-2H-pyrazolo[4,3- | | 2.00 (m, 1H), 1.84-1.73 (m, 1H), 1.57-1.48 (m, 2H), 1.36 (t, J = 5.9 Hz, 4H), 0.95 |
| | b]pyridin-2-yl)acetonitrile | | (t, J = 7.4 Hz, 3H), 0.75 (t, J = 7.3 Hz, 3H) ppm. MS: M/e 469(M + 1)⁺. |
| | | A132b | A132b (the later peak): ¹H NMR (400 MHz, CD₃OD) δ 7.93 (s, 1H), 7.64 (dd, J = 15.3, |
| | | (2 mg) | 8.3 Hz, 1H), 6.92 (ddd, J = 21.9, 14.1, 5.4 Hz, 2H), 5.55 (s, 1H), 5.47 (s, 2H), 4.00 (d, |
| | | | J = 6.6 Hz, 1H), 3.48 (d, J = 13.9 Hz, 1H), 3.43 (s, 3H), 3.35-3.25 (m, 2H), 3.12 (d, J = |
| | | | 10.3 Hz, 1H), 2.68 (d, J = 8.3 Hz, 1H), 2.35 (d, J = 12.2 Hz, 1H), 1.94 (s, 1H), 1.67 (tt, |
| | | | J = 14.2, 7.2 Hz, 2H), 1.59-1.48 (m, 1H), 1.32 (d, J = 6.6 Hz, 3H), 1.02 (t, J = 7.3 Hz, |
| | | | 3H), 0.65 (t, J = 7.3 Hz, 3H) ppm. MS: M/e 469(M + 1)⁺. |
| A135 | 2-(7-((2S,5R)-2,5-diethyl-4-(1-(6- | A135a | A135a (the earlier peak): ¹H NMR (400 MHz, CD₃OD) δ 8.72 (s, 1H), 8.09 (d, J = 7.9 |
| | (trifluoromethyl)pyridin-3- | (10 mg) | Hz, 1H), 7.94 (s, 1H), 7.82 (d, J = 8.0 Hz, 1H), 5.57 (s, 1H), 5.47 (s, 2H), 4.01-3.98 |
| | yl)ethyl)piperazin-1-yl)-4-methyl-5- | | (m 1H), 3.43 (s, 3H), 3.35-3.31 (m, 2H), 3.03-2.92 (m, 2H), 2.75 (br s, 1H), 2.37- |
| | oxo-4,5-dihydro-2H-pyrazolo[4,3- | | 2.35 (m, 1H), 2.15-2.05 (m, 1H), 1.86-1.82 (m, 1H), 1.62-1.50 (m, 2H), 1.41 (d, J = |
| | b]pyridin-2-yl)acetonitrile | | 6.5 Hz, 3H), 0.95-0.92 (m, 3H), 0.77-0.72 (m, 3H) ppm. MS: M/e 502 (M + 1)⁺. |
| | | A135b | A135b (the later peak): ¹H NMR (400 MHz, CD₃OD) δ 8.75 (s, 1H), 8.11 (d, J = 7.1 |
| | | (11 mg) | Hz, 1H), 7.93 (s, 1H), 7.80 (d, J = 8.1 Hz, 1H), 5.56 (s, 1H), 5.47 (s, 2H), 3.83-3.78 |
| | | | (m, 1H), 3.55-3.51 (m, 1H), 3.43 (s, 3H), 3.17-3.13 (m, 1H), 2.79-2.73 (m, 3H), |
| | | | 2.28-2.24 (m, 1H), 1.95-1.85 (m, 1H), 1.75-1.52 (m, 3H), 1.38 (d, J = 6.6 Hz, 3H), |
| | | | 1.03 (t, J = 7.5 Hz, 3H), 0.63 (t, J = 7.3 Hz, 3H) ppm. MS: M/e 502 (M + 1)⁺. |
| A138 | 2-(7-((2S,5R)-4-(1-(3,4-dihydro-2H- | 12 mg, 41% | ¹H NMR (400 MHz, CD₃OD) δ 7.92 (d, J = 2.2 Hz, 1H), 6.66 (s, 1H), 6.60 (s, 1H), |
| | benzo[b][1,4]oxazin-6-yl)ethyl)-2,5- | | 5.54 (s, 1H), 5.46 (s, 1H), 4.18 (dd, J = 8.9, 5.0 Hz, 2H), 3.43 (s, 3H), 3.36-3.32 (m, |
| | diethylpiperazin-1-yl)-4-methyl-5- | | 2H), 3.21-2.99 (m, 1H), 2.97-2.80 (m, 1H), 2.65-2.37 (m, 2H), 2.21-1.88 (m, |
| | oxo-4,5-dihydro-2H-pyrazolo[4,3- | | 3H), 1.65-1.46 (m, 3H), 1.29 (s, 3H), 1.25 (d, J = 6.5 Hz, 2H), 1.07-0.84 (m, 4H), |
| | b]pyridin-2-yl)acetonitrile | | 0.76-0.62 (m, 3H) ppm. MS: M/e 490 (M + 1)⁺ |
| A139 | 2-(7-((2S,5R)-2,5-diethyl-4-(1- | A139a (4 | A139a (the earlier peak): ¹H NMR (400 MHz, CD₃OD) δ 8.90-8.78 (m, 2H), 8.18- |
| | (quinoxalin-6-yl)ethyl)piperazin-1- | mg, 7%) | 7.90 (m, 4H), 6.28-5.90 (m, 1H), 5.55 (s, 1H), 5.46(s, 2H), 4.35-4.20 (m, 2H), 4.18- |
| | yl)-4-(2,2-difluoroethyl)-5-oxo-4,5- | | 4.01 (m, 1H), 3.40-3.33 (m, 2H), 3.20-2.83 (m, 2H), 2.51-2.30 (m, 1H), 2.28- |
| | dihydro-2H-pyrazolo[4,3-b]pyridin- | | 2.15 (m, 1H), 2.00-1.80 (m, 1H), 1.70-1.53 (m, 3H), 1.46 (d, J = 6.5 Hz, 3H), 0.99 |
| | 2-yl)acetonitrile | | (t, J = 7.4 Hz, 3H), 0.69 (t, J = 7.4 Hz, 3H) ppm. MS: M/e 535(M + 1)⁺. |
| | | A139b (4 | A139b (the later peak): ¹H NMR (400 MHz, CD₃OD) δ 8.85-8.78 (m, 2H), 8.20- |
| | | mg, 7%) | 7.95 (m, 4H), 6.28-5.90 (m, 1H), 5.56 (s, 1H), 5.45(s, 2H), 4.35-4.20 (m, 2H), 4.02- |
| | | | 3.85 (m, 1H), 3.60-3.50 (m, 1H), 3.33-3.18 (m, 1H), 2.80-2.60 (m, 2H), 2.51- |
| | | | 2.25 (m, 1H), 2.08-1.90 (m, 1H), 1.85-1.54 (m, 4H), 1.50-1.30 (m, 3H), 1.25- |
| | | | 0.97 (m, 3H), 0.69 (t, J = 7.4 Hz, 3H) ppm. MS: M/e 535(M + 1)⁺. |
| A140 | 2-(7-((2S,5R)-2,5-diethyl-4-(1-(5- | 4 mg, 8% | ¹HNMR (400 MHz, CD₃OD) δ 8.14-8.06 (m, 1H), 7.95-7.91 (m, 1H), 7.58-7.39 |
| | isopropoxypyridin-2- | | (m, 2H), 5.55 (s, 1H), 5.50-5.44 (m, 2H), 4.75-4.58 (m, 1H), 3.94-3.81 (m, 0.5H), |
| | yl)ethyl)piperazin-1-yl)-4-methyl-5- | | 3.75-3.66 (m, 0.5H), 3.55-3.46 (m, 0.5H), 3.43 (s, 3H), 3.31-3.26 (m, 2.5H), 3.20- |
| | oxo-4,5-dihydro-2H-pyrazolo[4,3- | | 3.10 (m, 0.5H), 3.06-2.98 (m, 0.5H), 2.92-2.83 (m, 0.5H), 2.72-2.63 (m, 0.5H), |
| | b]pyridin-2-yl)acetonitrile | | 2.41-2.25 (m, 1H), 2.19-1.46 (m, 4H), 1.41-1.27 (m, 9H), 1.02 (t, J = 7.6 Hz, |
| | | | 1.5H), 0.95 (t, J = 7.6 Hz, 1.5H), 0.71 (t, J = 7.6 Hz, 1.5H), 0.65 (t, J = 7.6 Hz, 1.5H) |
| | | | ppm. MS: M/e 492 (M + 1)⁺. |
| A141 | 2-(7-((2S,5R)-4-(1-(2-ethoxy-4- | 2 mg | ¹H NMR (400 MHz, CD₃OD) δ 7.92 (d, J = 3.2 Hz, 1H), 7.57-7.41 (m, 1H), 6.75- |
| | fluorophenyl)ethyl)-2,5- | | 6.59 (m, 2H), 5.54 (s, 1H), 5.47 (d, J = 3.2 Hz, 2H), 4.33-4.10 (m, 1H), 4.09-3.99 |
| | diethylpiperazin-1-yl)-4-methyl-5- | | (m, 2H), 3.47 (d, J = 13.2 Hz, 1H), 3.43 (s, 3H), 3.27-3.05 (m, 1H), 3.00-2.85 (m, |
| | oxo-4,5-dihydro-2H-pyrazolo[4,3- | | 1H), 2.61 (d, J = 8.8 Hz, 1H), 2.45-2.35 (m, 1H), 2.14-1.93 (m, 1H), 1.86-1.47 (m, |
| | b]pyridin-2-yl)acetonitrile | | 4H), 1.45-1.38 (m, 3H), 1.28-1.20 (m, 3H), 1.06-0.91 (m, 3H), 0.77-0.63 (m, 3H) |
| | | | ppm. MS: M/e 495 (M + 1)⁺. |
| A142 | 2-(7-((2S,5R)-2,5-diethyl-4-(1-(4- | 2 mg | ¹H NMR (400 MHz, CD3OD) δ 7.92 (d, J = 2.8 Hz, 1H), 7.58-7.42 (m, 1H), 6.78- |
| | fluoro-2- | | 6.58 (m, 2H), 5.54 (s, 1H), 5.47 (d, J = 3.2 Hz, 2H), 4.64-4.52 (m, 1H), 4.30-4.08 |
| | isopropoxyphenyl)ethyl)piperazin- | | (m, 1H), 3.52-3.43 (m, 1H), 3.43 (s, 3H), 3.29-3.23 (m, 1H), 3.14-2.83 (m, 2H), 2.65- |
| | 1-yl)-4-methyl-5-oxo-4,5-dihydro- | | 2.34 (m, 2H), 2.15-1.92 (m, 1H), 1.86-1.46 (m, 3H), 1.36-1.31 (m, 6H), 1.27-1.19 |
| | 2H-pyrazolo[4,3-b]pyridin-2- | | (m, 3H), 1.06-0.92 (m, 3H), 0.76-0.63 (m, 3H) ppm. MS: M/e 509 (M + 1)⁺. |
| | yl)acetonitrile | | |
| A146 | 2-(7-((2S,5R)-2,5-diethyl-4- | A146a (7 | MS: M/e 459 (M + 1)⁺. |
| | (1,2,3,4-tetrahydronaphthalen-1- | mg, 20%) | A146a: ¹H NMR (400 MHz, CD₃OD) δ 7.91 (s, 1H), 7.46-7.45 (m, 1H), 7.11-7.04 (m, |
| | yl)piperazin-1-yl)-4-methyl-5-oxo- | | 3H), 5.54 (s, 1H), 5.45 (s, 2H), 4.53-4.49 (m, 1H), 3.85 (s, 1H), 3.42-3.38 (m, 4H), |
| | 4,5-dihydro-2H-pyrazolo[4,3- | | 2.94-2.87 (m, 2H), 2.68-2.51 (m, 3H), 2.24-1.32 (m, 4H), 1.71-1.62 (m, 5H), 0.85-0.83 |
| | b]pyridin-2-yl)acetonitrile | | (m, 3H), 0.76-0.72 (m, 3H) ppm. |
| | | A146b (6 | A146b: ¹H NMR (400 MHz, CD₃OD) δ 7.94 (s, 1H), 7.53-7.49 (m, 1H), 7.10-7.05 (m, |
| | | mg, 18%). | 3H), 5.55-5.49 (m, 2H), 5.34-5.31 (m, 1H), 3.87-3.85 (m, 2H), 3.63-3.61 (m, 1H), 3.39 |
| | | | (s, 3H), 3.01-2.85 (m, 2H), 2.74-2.61 (m, 3H), 2.21-1.32 (m, 9H), 0.96-0.92 (m, 3H), |
| | | | 0.74-0.70 (m, 3H) ppm. |
| A148 | 2-(7-((2S,5R)-4-(1-(3,5- | 5 mg, 10% | ¹HNMR (400 MHz, CD₃OD) δ 8.38 (s, 1H), 7.95-7.90 (m, 1H), 7.62-7.52 (m, 1H), |
| | difluoropyridin-2-yl)ethyl)-2,5- | | 5.54-5.50 (m, 1H), 5.48-5.44 (m, 2H), 4.38-4.30 (m, 0.5H), 4.22-4.10 (m, 0.5H), |
| | diethylpiperazin-1-yl)-4-methyl-5- | | 3.54-3.46 (m, 0.5H), 3.43 (s, 3H), 3.31-3.26 (m, 2.5H), 3.15-3.02 (m, 1H), 2.90- |
| | oxo-4,5-dihydro-2H-pyrazolo[4,3- | | 2.76 (m, 1H), 2.54-2.31 (m, 1H), 1.96-1.75 (m, 1H), 1.71-1.51 (m, 3H), 1.50- |
| | b]pyridin-2-yl)acetonitrile | | 1.39 (m, 3H), 0.99 (t, J = 7.6 Hz, 1.5H), 0.90-0.76 (m, 3H), 0.66 (t, J = 7.6 Hz, 1.5H) |
| | | | ppm. MS: M/e 470 (M + 1)⁺. |
| A150 | 2-(7-((2S,5R)-4-(chroman-4-yl)-2,5- | A150a | A150a: ¹H NMR (400 MHz, CD₃OD) δ 7.94 (s, 1H), 7.31 (d, J = 8 Hz, 1H), 7.11 (t, J = |
| | diethylpiperazin-1-yl)-4-methyl-5- | (2 mg) | 4 Hz, 1H), 6.84 (t, J = 4 Hz, 1H), 6.74-6.72 (d, J = 8 Hz, 1H), 5.51-5.48 (m, 2 H), |
| | oxo-4,5-dihydro-2H-pyrazolo[4,3- | | 5.23-5.19 (m, 1H), 4.45-4.41 (m, 1H), 4.25-4.15 (m, 2H), 3.94-3.90 (m, 1H), 3.57-3.53 |
| | b]pyridin-2-yl)acetonitrile | | (m, 1H), 3.33-3.29 (s, 3H), 2.98-2.94 (m, 1H), 2.65-2.55 (m, 2H), 2.25-1.97 (m, 2H), |
| | | | 1.64-1.51 (m, 4H), 1.31-1.27 (m, 1H), 0.95 (t, J = 8 Hz, 3H), 0.71 (t, J = 8 Hz, 3H) |
| | | | ppm. MS: M/e 461 (M + 1)⁺. |
| | | A150b | A150b: ¹H NMR (400 MHz, CD₃OD) δ 7.93 (s, 1H), 7.40 (d, J = 8 Hz, 1H), 7.10 (t, J = |
| | | (3 mg) | 4 Hz, 1H), 6.84 (t, J = 4 Hz, 1H), 6.73 (d, J = 8 Hz, 1H), 5.56 (s, 1 H), 5.47 (s, 2H), |
| | | | 4.43-4.41 (m, 1H), 4.15-4.11 (m, 1H), 3.90-3.89 (m, 1H), 3.41-3.37 (m, 4H), 3.31 (s, |
| | | | 1H), 2.99-2.95 (m, 1H), 2.71-2.64 (m, 2H), 2.18-2.22 (m, 1H), 1.96-1.92 (m, 2H), 1.80- |
| | | | 1.62 (m, 3H), 1.29-1.25 (m, 1H), 0.91 (t, J = 8 Hz, 3H), 0.79 (t, J = 8 Hz, 3H) ppm. |
| | | | MS: M/e 461 (M + 1)⁺. |
| A151 | 2-(7-((2S,5R)-4-(1-(3- | 1 mg, | ¹H NMR (400 MHz, DMSO-d6) δ 8.14 (s, 1H), 7.99 (d, J = 2.1 Hz, 1H), 7.42-7.32 (m, |
| | ((dimethylamino)methyl)phenyl)ethyl)- | HCOOH | 3H), 7.28 (s, 1H), 5.62 (s, 2H), 5.40 (s, 1H), 4.04-3.40 (m, 4H), 3.27 (s, 3H), 3.20- |
| | 2,5-diethylpiperazin-1-yl)-4- | salt | 3.00 (m, 2H), 2.92 (d, J = 11.9 Hz, 0.5H), 2.75 (d, J = 11.9 Hz, 0.5H), 2.54 (s, 1H), |
| | methyl-5-oxo-4,5-dihydro-2H- | | 2.48-2.32 (m, 6H), 2.30 (s, 0.5H), 2.23 (d, J = 12.5 Hz, 0.5H), 2.06-1.99 (m, 0.5H), |
| | pyrazolo[4,3-b]pyridin-2- | | 1.88-1.81 (m, 0.5H), 1.66-1.54 (m, 1H), 1.52-1.40 (m, 2H), 1.27 (dd, J = 15.5, 6.6 Hz, |
| | yl)acetonitrile | | 3H), 0.99-0.83 (m, 3H), 0.63-0.44 (m, 3H) ppm. MS: M/e 490 (M + 1)+. |
| A153 | 2-(7-((2S,5R)-2,5-diethyl-4-(1-(4- | A153a | A153a (the earlier peak): ¹H NMR (400 MHz, DMSO-d6) δ 7.98 (s, 1H), 7.51 (t, J = |
| | fluoro-2- | (10 mg) | 7.9 Hz, 1H), 6.87 (dd, J = 11.5, 2.3 Hz, 1H), 6.76 (td, J = 9.6, 7.3 Hz, 1H), 5.61 (s, 2H), |
| | methoxyphenyl)ethyl)piperazin-1- | | 5.37 (s, 1H), 4.82 (s, 1H), 3.99 (q, J = 6.5 Hz, 1H), 3.84 (t, J = 5.9 Hz, 2H), 3.80 (s, |
| | yl)-4-(2-hydroxyethyl)-5-oxo-4,5- | | 3H), 3.58 (t, J = 5.6 Hz, 2H), 3.33 (s, 3H), 3.00 (d, J = 10.3 Hz, 1H), 2.53-2.51 (m, |
| | dihydro-2H-pyrazolo[4,3-b]pyridin- | | 0.5H), 2.48-2.44 (m, 0.5H), 2.24 (d, J = 12.1 Hz, 1H), 1.93 (s, 1H), 1.66-1.34 (m, |
| | 2-yl)acetonitrile | | 3H), 1.15 (d, J = 6.4 Hz, 3H), 0.95 (t, J = 6.8 Hz, 3H), 0.59 (t, J = 6.8 Hz, 3H) ppm. |
| | | | M/e 511 (M + 1)⁺ |
| | | A153b | A153b (the later peak): ¹H NMR (400 MHz, DMSO-d6) δ 7.98 (s, 1H), 7.43 (t, J = 8.0 |
| | | (5 mg) | Hz, 1H), 6.89 (dd, J = 11.4, 2.4 Hz, 1H), 6.78 (td, J = 11.4, 2.4 Hz, 1H), 5.61 (s, 2H), |
| | | | 5.37 (s, 1H), 4.82 (s, 1H), 4.16 (q, J = 6.5 Hz, 1H), 3.84 (t, J = 6.5 Hz, 2H), 3.80 (s, |
| | | | 3H), 3.57 (t, J = 5.8 Hz, 2H), 3.40-3.27 (m, 2H), 3.10 (d, J = 10.7 Hz, 1H), 2.86 (d, J = |
| | | | 11.3 Hz, 1H), 2.75 (d, J = 8.3 Hz, 1H), 2.31 (s, 1H), 2.06-1.94 (m, 1H), 1.63-1.54 (m, |
| | | | 1H), 1.45-1.38 (m, 2H), 1.20 (d, J = 6.4 Hz, 3H), 0.86 (t, J = 6.9 Hz, 3H), 0.64 (t, J = |
| | | | 7.2 Hz, 3H) ppm. M/e 511 (M + 1)⁺ |
| A155 | 2-(7-((2S,5R)-2,5-diethyl-4-(1- | Separated isomer | A155a (the earlier peak): ¹H NMR (400 MHz, DMSO-d6) δ 9.14 (s, 1H), 8.09 (dd, J = |
| | (quinoxalin-2-yl)ethyl)piperazin-1- | A155a | 15.7, 8.3 Hz, 2H), 7.99 (s, 1H), 7.90-7.80 (m, 2H), 5.62 (s, 2H), 5.39 (s, 1H), 3.96 (q, |
| | yl)-4-methyl-5-oxo-4,5-dihydro-2H- | (22 mg) | J = 6.9 Hz, 1H), 3.46 (d, J = 12.3 Hz, 1H), 3.34 (s, 1H), 3.27 (s, 3H), 3.12 (d, J = 9.7 |
| | pyrazolo[4,3-b]pyridin-2- | | Hz, 1H), 2.72 (d, J = 9.6 Hz, 1H), 2.55-2.51 (m, 1H), 2.16 (d, J = 12.0 Hz, 1H), 1.89- |
| | yl)acetonitrile | | 1.73 (m, 1H), 1.68-1.47 (m, 3H), 1.44 (d, J = 6.6 Hz, 3H), 0.97 (t, J = 7.1 Hz, 3H), 0.46 |
| | | | (t, J = 7.1 Hz, 3H) ppm. MS: M/e 485 (M + 1)+. |
| | | separated isomer | A155b (the later peak): ¹H NMR (400 MHz, DMSO-d6) δ 9.11 (s, 1H), 8.09 (t, J = 7.6 |
| | | A155b | Hz, 2H), 7.98 (s, 1H), 7.91-7.77 (m, 2H), 5.61 (s, 2H), 5.40 (s, 1H), 4.21 (q, J = 6.7 |
| | | (27 mg) | Hz, 1H), 3.34 (s, 2H), 3.27 (s, 4H), 2.87 (dd, J = 37.2, 10.8 Hz, 2H), 2.49-2.42 (m, |
| | | | 1H), 1.95-1.81 (m, 1H), 1.68-1.53 (m, 3H), 1.49 (d, J = 6.4 Hz, 3H), 0.70 (q, J = 7.0 |
| | | | Hz, 6H) ppm. MS: M/e 485 (M + 1)+. |
| A157 | 2-(7-((2S,5R)-2,5-diethyl-4-(1-(2- | 16 mg, 34% | ¹H NMR (400 MHz, CD₃OD) δ 7.93 (d, J = 2.8 Hz, 1H), 7.81-7.68 (m, 1H), 7.30- |
| | methoxy-4- | | 7.17 (m, 2H), 5.55 (s, 1H), 5.47 (d, J = 3.2 Hz, 2H), 4.44-4.15 (m, 1H), 3.90 (s, 3H), |
| | (trifluoromethyl)phenyl)ethyl)piperazin- | | 3.56-3.46 (m, 1H), 3.43 (s, 3H), 3.28-3.09 (m, 1H), 3.03-2.87 (m, 1H), 2.65 (d, J = |
| | 1-yl)-4-methyl-5-oxo-4,5- | | 14.4 Hz, 1H), 2.45-2.27 (m, 1H), 2.13-1.49 (m, 5H), 1.34-1.20 (m, 3H), 1.08- |
| | dihydro-2H-pyrazolo[4,3-b]pyridin- | | 0.91 (m, 3H), 0.77-0.62 (m, 3H) ppm. MS: M/e 531 (M + 1)⁺. |
| | 2-yl)acetonitrile | | |
| A160 | 2-(7-((2S,5R)-2,5-diethyl-4-(1-(4- | 5 mg, 11% | ¹H NMR (400 MHz, CD₃OD) δ 7.93 (s, 1H), 7.26 (s, 2H), 6.86 (s, 2H), 5.54 (s, 1H), |
| | isopropoxyphenyl)ethyl)piperazin- | | 5.47 (s, 2H), 4.58 (s, 1H), 3.74-3.58 (m, 1H), 3.50 (s, 1H), 3.43 (s, 4H), 3.13 (s, 1H), |
| | 1-yl)-4-methyl-5-oxo-4,5-dihydro- | | 2.89-2.74 (m, 1H), 2.64-2.50 (m, 1H), 2.50-2.35 (m, 1H), 2.01-1.86 (m, 1H), |
| | 2H-pyrazolo[4,3-b]pyridin-2- | | 1.60 (s, 2H), 1.30 (t, J = 5.3 Hz, 9H), 0.96 (s, 4H), 0.62 (s, 3H) ppm. MS: M/e 491 |
| | yl)acetonitrile | | (M + 1)⁺. |
| A163 | 2-(7-((2S,5R)-2,5-diethyl-4-(1-(4- | 12 mg, 16% | ¹H NMR (400 MHz, CD₃OD) δ 7.93 (s, 1H), 6.92 (dd, J = 17.1, 7.9 Hz, 1H), 6.36- |
| | fluoro-2- | | 6.15 (m, 2H), 5.57 (d, J 9.5 Hz, 1H), 5.47 (s, 2H), 3.80-3.64 (m, 1H), 3.43 (s, 3H), |
| | (methylamino)phenyl)ethyl)piperazin- | | 3.12-3.01 (m, 1H), 2.82 (s, 1H), 2.76 (s, 2H), 2.72-2.58 (m, 1H), 2.56-2.47 (m, |
| | 1-yl)-4-methyl-5-oxo-4,5- | | 1H), 1.90-1.77 (m, 1H), 1.64-1.55 (m, 2H), 1.54-1.47 (m, 1H), 1.41 (d, J = 5.8 Hz, |
| | dihydro-2H-pyrazolo[4,3-b]pyridin- | | 3H), 1.29 (s, 3H), 1.05-0.95 (m, 3H), 0.73-0.62 (m, 3H) ppm. MS: M/e 480 (M + 1)+ |
| | 2-yl)acetonitrile | | |
| A165 | N-(2-(1-((2R,5S)-4-(2- | 1 mg, 2% | ¹H NMR (400 MHz, CD₃OD) δ 8.13-7.91 (m, 2H), 7.27-7.22 (m, 2H), 7.15-7.08 (m, |
| | (cyanomethyl)-4-methyl-5-oxo-4,5- | | 1H), 5.60 (d, J = 5.9 Hz, 1H), 5.48 (s, 2H), 3.88-3.84 (m, 1H), 3.51 (d, J = 22.1 Hz, |
| | dihydro-2H-pyrazolo[4,3-b]pyridin- | | 1H), 3.44 (s, 3H), 2.87-2.68 (m, 2H), 2.49 (d, J = 12.8 Hz, 1H), 2.20 (d, J = 11.1 Hz, |
| | 7-yl)-2,5-diethylpiperazin-1- | | 3H), 2.03 (s, 1H), 1.87 (s, 1H), 1.77-1.50 (m, 3H), 1.43 (dd, J = 14.6, 6.8 Hz, 3H), |
| | yl)ethyl)phenyl)acetamide | | 1.06 (t, J = 7.4 Hz, 2H), 0.97-0.93 (m, 2H), 0.72-0.60 (m, 3H) ppm. MS: M/e 490 |
| | | | (M + 1)⁺. |
| A167 | 2-(4-(cyclopropylmethyl)-7- | 22 mg, 26% | ¹H NMR (400 MHz, CD₃OD) δ 8.87 (d, J = 5.8 Hz, 2H), 8.09 (d, J = 6.6 Hz, 2H), 8.03 |
| | ((2S,5R)-2,5-diethyl-4-(1- | | (d, J = 8.2 Hz, 1H), 7.99 (d, J = 3.0 Hz, 1H), 5.56 (s, 1H), 5.45 (d, J = 5.7 Hz, 2H), 4.08- |
| | (quinoxalin-6-yl)ethyl)piperazin-1- | | 3.91 (m, 1H), 3.84 (d, J = 7.0 Hz, 2H), 3.65-3.42 (m, 1H), 3.27-3.16 (m, 1H), 3.12- |
| | yl)-5-oxo-4,5-dihydro-2H- | | 2.94 (m, 1H), 2.82-2.71 (m, 1H), 2.44-2.32 (m, J = 12.4 Hz, 1H), 2.03-1.86 (m, |
| | pyrazolo[4,3-b]pyridin-2- | | 1H), 1.76-1.57 (m, 3H), 1.49-1.42 (m, 3H), 1.37-1.21 (m, 3H), 1.07 (t, J = 7.1 Hz, |
| | yl)acetonitrile | | 1H), 0.99 (t, J = 7.0 Hz, 1H), 0.69 (t, J = 7.2 Hz, 1H), 0.58-0.49 (m, 3H), 0.49-0.39 |
| | | | (m, 3H) ppm. MS: M/e 525 (M + 1)+ |
| A173 | methyl 4-(2-(cyanomethyl)-4- | A173a | A173a: ¹H NMR (400 MHz, DMSO-d6) δ 8.94 (d, J = 2.8 Hz, 2H), 8.09 (d, J = 8.7 Hz, |
| | methyl-5-oxo-4,5-dihydro-2H- | (17 mg) | 1H), 7.99 (s, 1H), 7.91 (s, 1H), 7.88 (d, J = 8.7 Hz, 1H), 5.60 (s, 2H), 5.50 (s, 1H), 4.51 |
| | pyrazolo[4,3-b]pyridin-7-yl)-1-(1- | | (d, J = 10.9 Hz, 1H), 4.25 (q, J = 6.6 Hz, 1H), 4.18 (d, J = 10.6 Hz, 1H), 3.50 (s, 3H), |
| | (quinoxalin-6-yl)ethyl)piperazine-2- | | 3.42-3.38 (m, 1H), 3.38-3.35 (m, 1H), 3.32-3.30 (m, 1H), 3.30-3.28 (m, 1H), 3.27 (s, |
| | carboxylate | | 3H), 2.93 (d, J = 12.3 Hz, 1H), 1.47 (d, J = 6.6 Hz, 3H) ppm. MS: M/e 487 (M + 1)+. |
| | | A173b | A173b: ¹H NMR (400 MHz, DMSO-d6) δ 8.94 (dd, J = 7.1, 1.7 Hz, 2H), 8.10 (d, J = |
| | | (26 mg) | 8.7 Hz, 1H), 8.05 (s, 1H), 8.01 (s, 1H), 7.98 (d, J = 8.5 Hz, 1H), 5.63 (s, 2H), 5.51 (s, |
| | | | 1H), 4.90 (d, J = 13.8 Hz, 1H), 4.27 (q, J = 6.7 Hz, 1H), 4.10 (t, J = 3.6 Hz, 1H), 3.84 |
| | | | (d, J = 9.0 Hz, 1H), 3.63-3.56 (m, 4H), 3.29 (s, 3H), 3.06 (t, J = 11.0 Hz, 2H), 2.46- |
| | | | 2.38 (m, 1H), 1.34 (d, J = 6.6 Hz, 3H) ppm. MS: M/e 487 (M + 1)+. |
| A174 | 2-(7-((2S,5R)-2,5-diethyl-4-(1-(1- | 4 mg, 9% | ¹H NMR (400 MHz, CD₃OD) δ 7.92 (s, 1H), 7.30-7.15 (m, 1H), 7.10-6.95 (m, 1H), |
| | ethyl-1H-imidazol-2- | | 5.59-5.51 (m, 1H), 5.49-5.40 (m, 2H), 4.45-4.10 (m, 4H), 3.59-3.34 (m, 4H), |
| | yl)ethyl)piperazin-1-yl)-4-methyl-5- | | 3.08-2.61 (m, 2H), 2.48-2.20 (m, 1H), 1.98-1.62 (m, 2H), 1.61-1.26 (m, 9H), 1.05- |
| | oxo-4,5-dihydro-2H-pyrazolo[4,3- | | 0.76 (m, 3H), 0.74-0.62 (m , 3H) ppm. MS: M/e 451 (M + 1)⁺. |
| | b]pyridin-2-yl)acetonitrile | | |
| A175 | 2-(7-((2S,5R)-4-(1-(2-cyclopropyl- | 24 mg, 32% | ¹H NMR (400 MHz, CD₃OD) δ 7.93 (s, 1H), 7.64-7.44 (m, 1H), 6.88 (td, J = 8.5, 2.7 |
| | 4-fluorophenyl)ethyl)-2,5- | | Hz, 1H), 6.69 (dd, J = 19.5, 10.6 Hz, 1H), 5.56 (d, J = 4.6 Hz, 1H), 5.47 (s, 2H), 4.28 |
| | diethylpiperazin-1-yl)-4-methyl-5- | | (s, 1H), 3.49 (d, J = 13.1 Hz, 1H), 3.43 (s, 3H), 3.19-3.06 (m, 1H), 2.90-2.64 (m, |
| | oxo-4,5-dihydro-2H-pyrazolo[4,3- | | 2H), 2.63-1.80 (m, 4H), 1.62 (s, 3H), 1.32 (dd, J = 16.0, 6.4 Hz, 3H), 1.09-0.89 (m, |
| | b]pyridin-2-yl)acetonitrile | | 5H), 0.75-0.53 (m, 5H) ppm. MS: M/e 491 (M + 1)⁺ |
| A176 | 2-(7-((2S,5R)-2,5-diethyl-4-(1-(1- | 8 mg, 16% | ¹HNMR (400 MHz, CD₃OD) δ 7.92 (d, J = 5.2 Hz, 1H), 7.48 (d, J = 8.0 Hz, 1H), 7.34 |
| | ethyl-1H-indol-2-yl)ethyl)piperazin- | | (t, J = 7.2 Hz, 1H), 7.11 (t, J = 7.2 Hz, 1H), 6.99 (t, J = 7.2 Hz, 1H), 6.40 6.33 (m, 1H), |
| | 1-yl)-4-methyl-5-oxo-4,5-dihydro- | | 5.57-5.41 (m, 3H), 4.67-4.02 (m, 3H), 3.56-3.37 (m, 1H), 3.42 (s, 3H), 3.33-3.27 |
| | 2H-pyrazolo[4,3-b]pyridin-2- | | (m, 2H), 3.10-2.84 (m, 1.5H), 2.83-2.71 (m, 0.5H), 2.68-2.54 (m, 1H), 2.03-1.60 |
| | yl)acetonitrile | | (m, 3H), 1.59-1.46 (m, 4H), 1.41 (t, J = 7.2 Hz, 1.5H), 1.34 (t, J = 7.2 Hz, 1.5H), 1.02 |
| | | | (t, J = 7.6 Hz, 1.5H), 0.78 (t, J = 8.0 Hz, 1.5H), 0.75-0.64 (m, 3H) ppm. MS: M/e 500 |
| | | | (M + 1)⁺. |
| A178 | 2-(4-methyl-7-((3S)-3-methyl-4-(1- | 25 mg | ¹H NMR (400 MHz, DMSO-d6) δ 8.93 (dt, J = 5.1, 2.1 Hz, 2H), 8.16 (s, 1H), 8.08 (d, |
| | (quinoxalin-6-yl)ethyl)piperazin-1- | | J = 8.6 Hz, 1H), 8.05-7.89 (m, 3H), 5.62 (d, J = 9.5 Hz, 2H), 5.47 (d, J = 12.6 Hz, |
| | yl)-5-oxo-4,5-dihydro-2H- | | 1H), 4.27 (q, J = 6.6 Hz, 1H), 3.99 (d, J = 11.3 Hz, 0.5H), 3.81 (s, 0.5H), 3.69 (s, 1H), |
| | pyrazolo[4,3-b]pyridin-2- | | 3.61 (d, J = 9.8 Hz, 0.5H), 3.54-3.49 (m, 0.5H), 3.45-3.41 (m, 0.5H), 3.27 (d, J = 11.7 |
| | yl)acetonitrile | | Hz, 3H), 3.23-3.19 (m, 0.5H), 3.14 (s, 0.5H), 3.00 (s, 0.5H), 2.59-2.52 (m, 1H), 2.43- |
| | | | 2.35 (m, 1H), 1.46-1.42 (m, 3H), 1.19-1.13 (m, 3H) ppm. MS: M/e 443 (M + 1)+. |
| A184 | 2-(7-(3-(hydroxymethyl)-4-(1- | 2 mg | ¹H NMR (400 MHz, DMSO-d6) δ 8.93 (d, J = 5.8 Hz, 2H), 8.13-7.92 (m, 4H), 5.61 |
| | (quinoxalin-6-yl)ethyl)piperazin-1- | | (s, 2H), 5.46 (d, J = 21.7 Hz, 1H), 4.45 (dd, J = 11.1, 6.6 Hz, 1H), 3.92 (d, J = 12.1 Hz, |
| | yl)-4-methyl-5-oxo-4,5-dihydro-2H- | | 0.5H), 3.82-3.71 (m, 2.5H), 3.66-3.43 (m, 3H), 3.42-3.32 (m, 1H), 3.28 (d, J = 9.8 |
| | pyrazolo[4,3-b]pyridin-2- | | Hz, 3H), 3.04-2.96 (m, 1H), 2.67 (s, 0.5H), 2.60-2.51 (m, 1H), 2.48-2.44 (m, 0.5H), |
| | yl)acetonitrile | | 1.46 (dd, J = 15.6, 6.6 Hz, 3H) ppm. MS: M/e 459 (M + 1)⁺. |
| A185 | 2-(7-((2S,5R)-2,5-diethyl-4-(1-(3- | A185a | A185a (the earlier peak): ¹H NMR (400 MHz, CDCl₃) δ 8.41 (s, 1H), 8.04 (d, J = 4 Hz, |
| | ethyl-3H-imidazo[4,5-b]pyridin-2- | (7 mg) | 1H), 7.39 (s, 1H), 7.26 (s, 1H), 5.60 (s, 1H), 5.14-5.09 (m, 2H), 4.79-4.75 (m, 1H), |
| | yl)ethyl)piperazin-1-yl)-4-methyl-5- | | 4.47-4.32 (m, 3H), 3.55-3.51 (m, 1H), 3.42 (s, 3H), 3.04-2.85 (m, 2H), 2.45-2.41 (m, |
| | oxo-4,5-dihydro-2H-pyrazolo[4,3- | | 1H), 2.33-1.82 (m, 3H), 1.65 (s, 3H), 1.52-1.48 (m, 4H), 0.99-0.94 (m, 3H), 0.92-0.88 |
| | b]pyridin-2-yl)acetonitrile | | (m, 1H), 0.65-0.61 (m, 3H) ppm. MS: M/e 502 (M + 1)⁺. |
| | | A185b | A185b (the later peak): ¹H NMR (400 MHz, CDCl₃) δ 8.42 (s, 1H), 8.07-8.03 (m, 1H), |
| | | (9 mg) | 7.39 (s, 1H), 7.27 (s, 1H), 5.63 (s, 1H), 5.13 (s, 2H), 4.73-4.69 (m, 1H), 4.54-4.46 (m, |
| | | | 3H), 3.43-3.39 (m, 5H), 2.99-2.95 (m, 2H), 2.56-2.52 (m, 1H), 1.85-1.83 (m, 2H), 1.79- |
| | | | 1.69 (m, 4H), 1.57-1.45 (m, 4H), 0.79-0.74(m, 6H) ppm. MS: M/e 502 (M + 1)⁺. |
| A186 | 2-(but-2-yn-1-yl)-7-((2S,5R)-2,5- | 21 mg, 21% | ¹H NMR (400 MHz, CDCl₃) δ 7.82 (s, 1H), 7.45 (s, 1H), 7.43-7.41 (m, 1H), 7.34-7.31 |
| | diethyl-4-(1-(1-ethyl-1H- | | (m, 2H), 5.56 (s, 1H), 4.94-4.90 (m, 2H), 4.67-4.46 (m, 4H), 3.43 (s, 3H), 3.32-3.29 |
| | benzo[d]imidazol-2- | | (m, 1H), 2.94 (s, 2H), 2.42 (s, 1H), 1.88 (s, 3H), 1.86-1.82 (m, 1H), 1.80-1.65 (m, 7H), |
| | yl)ethyl)piperazin-1-yl)-4-methyl- | | 1.54-1.51 (m, 3H), 0.80-0.79 (m, 3H), 0.72 (t, J = 8 Hz, 3H) ppm. MS: M/e 514 (M + 1)⁺. |
| | 2,4-dihydro-5H-pyrazolo[4,3- | | |
| | b]pyridin-5-one | | |
| A187 | 2-(7-((2S,5R)-4-(1-(5-chloro-1- | A187a | A187a (the earlier peak): ¹H NMR (400 MHz, CD₃OD) δ 7.93 (s, 1H), 6.88 (s, 1H), |
| | ethyl-1H-imidazol-2-yl)ethyl)-2,5- | (1 mg) | 5.51 (s, 1H), 5.48 (s, 2H), 4.55-4.01 (m, 4H), 3.55-3.44 (m, 1H), 3.43 (s, 3H), 3.05- |
| | diethylpiperazin-1-yl)-4-methyl-5- | | 2.95 (m, 1H), 2.94-2.30 (m, 2H), 2.47-2.29 (m, 1H), 1.80-1.50 (m, 4H), 1.48- |
| | oxo-4,5-dihydro-2H-pyrazolo[4,3- | | 1.25 (m, 6H), 1.05-0.85 (m, 3H), 0.80-0.65 (m, 3H) ppm. MS: M/e 485 (M + 1)⁺. |
| | b]pyridin-2-yl)acetonitrile | A187b | A187b (the later peak): ¹H NMR (400 MHz, CD₃OD) δ 7.92 (s, 1H), 6.86 (s, 1H), 5.55 |
| | | (1 mg) | (s, 1H), 5.47 (s, 2H), 4.45-4.10 (m, 4H), 3.50-3.34 (m, 5H), 2.98-2.70 (m, 2H), |
| | | | 2.55-2.48 (m, 1H), 1.90-1.60 (m, 2H), 1.58-1.30 (m, 8H), 0.89-0.75 (m, 6H) ppm. |
| | | | MS: M/e 485 (M + 1)⁺. |
| A188 | 2-(7-((2S,5R)-2,5-diethyl-4-(1-(4- | 1 mg | ¹H NMR (400 MHz, CD₃OD) δ 7.92 (s, 1H), 7.68-7.47 (m, 1H), 7.28-7.12 (m, 1H), |
| | fluoro-2-(1- | | 7.02-6.80 (m, 1H), 5.62-5.47 (m, 1H), 5.46 (s, 2H), 5.28-5.16 (m, 1H), 4.05-3.90 |
| | hydroxyethyl)phenyl)ethyl)piperazin- | | (m, 1H), 3.57-3.33 (m, 5H), 3.23-2.98 (m, 1H), 2.80-2.60 (m, 2H), 2.52-2.25 (m, |
| | 1-yl)-4-methyl-5-oxo-4,5- | | 1H), 2.18-1.51 (m , 4H), 1.49-1.20 (m, 7H), 1.08-0.80 (m, 3H), 0.75-0.51 (m, 3H) |
| | dihydro-2H-pyrazolo[4,3-b]pyridin- | | ppm. MS: M/e 495 (M + 1)⁺. |
| | 2-yl)acetonitrile | | |
| A190 | 2-(7-((2S,5R)-2,5-diethyl-4-(1- | A190a | A190a: ¹H NMR (400 MHz, CD₃OD) δ 8.88 (d, J = 2.9 Hz, 2H), 8.11 (s, 2H), 8.05 (s, |
| | (quinoxalin-6-yl)ethyl)piperazin-1- | (9 mg, 30%) | 1H), 8.01 (d, J = 8.6 Hz, 1H), 5.55 (s, 1H), 5.44 (s, 2H), 5.19 (s, 1H), 4.07 (d, J = 6.2 |
| | yl)-4-isopropyl-5-oxo-4,5-dihydro- | | Hz, 1H), 3.32 (s, 2H), 3.09 (d, J = 12.5 Hz, 1H), 2.95 (d, J = 7.9 Hz, 1H), 2.90-2.58 |
| | 2H-pyrazolo[4,3-b]pyridin-2- | | (m, 1H), 2.42 (s, 1H), 2.24-2.14 (m, 1H), 1.90-1.81 (m, 1H), 1.66-1.58 (m, 2H), |
| | yl)acetonitrile | | 1.48-1.42 (m, 9H), 0.98 (t, J = 7.3 Hz, 3H), 0.68 (t, J = 7.3 Hz, 3H) ppm. MS: M/e |
| | | | 513 (M + 1)⁺ |
| | | A190b | A190b: ¹H NMR (400 MHz, CD₃OD) δ 8.87 (d, J = 5.2 Hz, 2H), 8.12-8.04 (m, 4H), |
| | | (6 mg, 20%) | 5.54 (s, 1H), 5.45 (s, 2H), 5.19 (s, 1H), 3.92 (d, J = 6.5 Hz, 1H), 3.56 (d, J = 13.2 Hz, |
| | | | 1H), 3.23 (d, J = 11.3 Hz, 2H), 2.75 (d, J = 9.4 Hz, 1H), 2.36 (d, J = 12.1 Hz, 1H), 1.76- |
| | | | 1.60 (m, 3H), 1.51-1.39 (m, 11H), 1.06 (t, J = 7.2 Hz, 3H), 0.56 (t, J = 7.3 Hz, 3H) |
| | | | ppm. MS: M/e 513 (M + 1)⁺ |
| A191 | 2-(7-((2S,5R)-4-(1-(4-chloro-1- | A191a | A191a: ¹H NMR (400 MHz, CD₃OD) δ 7.93 (s, 1H), 7.09 (s, 1H), 5.53 (s, 1H), 5.48 (s, |
| | ethyl-1H-imidazol-2-yl)ethyl)-2,5- | (2 mg) | 2H), 4.40-4.00 (m, 4H), 3.55-3.45 (m, 1H), 3.43 (s, 3H), 3.05-2.95 (m, 1H), 2.80- |
| | diethylpiperazin-1-yl)-4-methyl-5- | | 2.65 (m, 2H), 2.42-2.25 (m, 1H), 1.95-1.49 (m, 4H), 1.48-1.30 (m, 6H), 1.05- |
| | oxo-4,5-dihydro-2H-pyrazolo[4,3- | | 0.90 (m, 3H), 0.80-0.60 (m, 3H) ppm. MS: M/e 485.1 (M + 1)⁺. |
| | b]pyridin-2-yl)acetonitrile | A191b | A191b: ¹H NMR (400 MHz, CD₃OD) δ 7.92 (s, 1H), 7.11 (s, 1H), 5.56 (s, 1H), 5.47 |
| | | (2 mg) | (s, 2H), 4.30-4.08 (m, 3H), 3.51-3.34 (m, 4H), 2.98-2.60 (m, 3H), 2.48-2.40 (m, |
| | | | 1H), 1.95-1.60 (m, 2H), 1.58-1.30 (m, 9H), 0.89-0.70 (m, 6H) ppm. MS: M/e 485.1 |
| | | | (M + 1)⁺. |
| A192 | 2-(7-((2S,5R)-2,5-diethyl-4-(1-(1- | 0.68 mg | ¹H NMR (400 MHz, CD₃OD) δ 7.93 (d, J = 5.3 Hz, 1H), 7.66 (s, 1H), 7.57 (s, 1H), |
| | propyl-1H-benzo[d]imidazol-2- | | 7.30 (s, 2H), 5.53 (d, J = 18.4 Hz, 1H), 5.47 (d, J = 6.9 Hz, 2H), 4.59-4.50 (m, 1H), |
| | yl)ethyl)piperazin-1-yl)-4-methyl-5- | | 4.46 (s, 2H), 3.46 (s, 1H), 3.43 (s, 4H), 3.13 (s, 1H), 3.01 (s, 1H), 2.81 (s, 2H), 1.94 (s, |
| | oxo-4,5-dihydro-2H-pyrazolo[4,3- | | 2H), 1.89-1.78 (m, 2H), 1.64 (s, 2H), 1.62-1.58 (m, 3H), 1.29 (s, 3H), 1.05 (dd, J = |
| | b]pyridin-2-yl)acetonitrile | | 14.0, 6.9 Hz, 3H), 0.77 (s, 3H) ppm. MS: M/e 514 (M + 1)⁺ |
| A193 | 2-(7-((2S,5R)-2,5-diethyl-4-(1-(1- | 0.53 mg, 5% | ¹H NMR (400 MHz, CD₃OD) δ 7.93 (d, J = 3.2 Hz, 1H), 7.75 (s, 1H), 7.69-7.59 (m, |
| | isopropyl-1H-benzo[d]imidazol-2- | | 1H), 7.26 (s, 2H), 5.58 (s, 1H), 5.52 (s, 1H), 5.47 (d, J = 6.9 Hz, 2H), 4.43 (s, 1H), 3.46 |
| | yl)ethyl)piperazin-1-yl)-4-methyl-5- | | (s, 2H), 3.43 (s, 3H), 3.13 (s, 1H), 3.04 (s, 1H), 2.97-2.91 (m, 1H), 2.80 (s, 1H), 1.73- |
| | oxo-4,5-dihydro-2H-pyrazolo[4,3- | | 1.69 (m, 3H), 1.67 (d, J = 6.8 Hz, 2H), 1.64 (d, J = 6.8 Hz, 2H), 1.59 (d, J = 7.2 Hz, |
| | b]pyridin-2-yl)acetonitrile | | 3H), 1.29 (s, 3H), 1.02 (s, 3H), 0.76-0.68 (m, 3H) ppm. MS: M/e 515 (M + 1)⁺ |
| A195 | 2-(7-((2S,5R)-4-(1-(2-fluoro-4- | 16 mg, 18% | ¹H NMR (400 MHz, CDCl₃) δ 7.79-7.70 (m, 1H), 7.44-7.39 (m, 2H), 7.32-7.26 (m, |
| | (trifluoromethyl)phenyl)ethyl)-2,5- | | 1H), 5.65-5.61 (m, 1H), 5.16-1.12 (m, 2H), 4.74-4.62 (m, 1H), 4.13-4.03 (m, 1H), 3.98- |
| | dimethylpiperazin-1-yl)-4-methyl-5- | | 3.95 (m, 1H), 3.40-3.36 (m, 1H), 3.35 (s, 3H), 3.31-3.30 (m, 0.5H), 2.83-2.76 (m, |
| | oxo-4,5-dihydro-2H-pyrazolo[4,3- | | 1.5H), 2.15-2.12 (m, 1H), 1.37-1.35 (m, 4H), 1.33-1.30 (m, 4H), 1.21-1.14 (m, 1H) |
| | b]pyridin-2-yl)acetonitrile | | ppm. MS: M/e 491 (M + 1)⁺. |
| A197 | 2-(7-((2S,5R)-2,5-diethyl-4-(1-(2- | separated isomer | A197a (the earlier peak): ¹H NMR (400 MHz, CD₃OD) δ 8.01 (d, J = 8.0 Hz, 1H), 7.93 |
| | (trifluoromethyl)phenyl)ethyl)piperazin- | A197a | (s, 1H), 7.63-7.60 (m, 2H), 7.41-7.39 (m, 1H), 5.55 (s, 1H), 5.47 (s, 2H), 4.06-4.05 |
| | 1-yl)-4-methyl-5-oxo-4,5- | (12 mg) | (m, 1H), 3.51-3.50 (m, 1H), 3.43 (s, 3H), 3.22-3.19 (m, 1H), 2.73-2.63 (m, 2H), |
| | dihydro-2H-pyrazolo[4,3-b]pyridin- | | 2.19-2.16 (m, 1H), 1.93 (br s, 1H), 1.74-1.50 (m, 3H), 1.31 (d, J = 6.4 Hz, 3H), 1.05- |
| | 2-yl)acetonitrile | | 1.03 (m, 3H), 0.58-0.56 (m, 3H) ppm. MS: M/e 501 (M + 1)⁺. |
| | | separated isomer | A197b (the later peak): ¹H NMR (400 MHz, CD₃OD) δ 8.04 (d, J = 8.0 Hz, 1H), 7.92 |
| | | A197b | (s, 1H), 7.63-7.60 (m, 2H), 7.41-7.39 (m, 1H), 5.56 (s, 1H), 5.46 (s, 2H), 4.20-4.19 |
| | | (5 mg) | (m, 1H), 3.43 (s, 3H), 3.10-3.09 (m, 2H), 2.92-2.75 (m, 2H), 2.32-2.21 (m, 2H), |
| | | | 1.89-1.86 (m, 1H), 1.57-1.53 (m, 2H), 1.29 (d, J = 6.4 Hz, 3H), 1.01-0.99 (m, 3H), |
| | | | 0.72-0.70 (m, 3H) ppm. MS: M/e 501 (M + 1)⁺. |
| A198 | 4-(2-(cyanomethyl)-4-methyl-5- | separated isomer | A198a (the earlier peak): ¹H NMR (400 MHz, DMSO-d6) δ 8.94 (d, J = 5.8 Hz, 2H), |
| | oxo-4,5-dihydro-2H-pyrazolo[4,3- | A198a | 8.08 (d, J = 8.6 Hz, 1H), 7.98 (d, J = 3.3 Hz, 2H), 7.92 (d, J = 8.6 Hz, 1H), 7.58 (s, 1H), |
| | b]pyridin-7-yl)-1-(1-(quinoxalin-6- | (8 mg) | 7.28 (s, 1H), 5.60 (s, 2H), 5.37 (s, 1H), 4.26 (q, J = 6.7 Hz, 1H), 3.95 (d, J = 10.9 Hz, |
| | yl)ethyl)piperazine-2-carboxamide | | 1H), 3.91-3.83 (m, 1H), 3.79 (dd, J = 12.6, 6.7 Hz, 1H), 3.57 (t, J = 9.9 Hz, 1H), 3.24 = |
| | | | (s, 3H), 3.20 (s, 1H), 3.15 (dd, J = 6.4, 3.9 Hz, 1H), 2.33 (t, J = 9.2 Hz, 1H), 1.53 (d, J = |
| | | | 6.8 Hz, 3H) ppm. MS: M/e 472 (M + 1)⁺. |
| | | separated isomer | A198b (the later peak): ¹H NMR (400 MHz, DMSO-d6) δ 8.94 (d, J = 5.0 Hz, 2H), |
| | | A198b | 8.22-7.99 (m, 5H)(contained 1 eq HCOOH), 7.79 (s, 1H), 7.27 (s, 1H), 5.63 (s, 2H), |
| | | (13 mg) | 5.49 (s, 1H), 4.32-4.20 (m, 2H), 3.88-3.78 (m, 1H), 3.76-3.67 (m, 1H), 3.49 (d, J = |
| | | | 3.9 Hz, 1H), 3.29 (s, 3H), 3.28-3.23 (m, 1H), 2.69-2.62 (m, 1H), 2.60-2.55 (m, 1H), |
| | | | 1.44 (d, J = 6.7 Hz, 3H) ppm. MS: M/e 472 (M + 1)⁺. |
| A199 | 2-(7-((2S,5R)-4-(1-(4-cyclopropyl- | separated isomer | A199a (the earlier peak): ¹H NMR (400 MHz, CD₃OD) δ 7.93 (s, 1H), 7.41 (d, J = 8.0 |
| | 2-methoxyphenyl)ethyl)-2,5- | A199a | Hz, 1H), 6.67 (s, 2H), 5.54 (s, 1H), 5.47 (s, 2H), 4.15 (s, 1H), 3.81 (s, 3H), 3.49 (s, 1H), |
| | diethylpiperazin-1-yl)-4-methyl-5- | (0.4 mg) | 3.43 (s, 4H), 2.79 (s, 2H), 2.65 (s, 1H), 2.47-2.40 (m, 1H), 1.90 (s, 1H), 1.60 (s, 4H), |
| | oxo-4,5-dihydro-2H-pyrazolo[4,3- | | 1.23 (s, 4H), 1.01 (s, 3H), 0.97-0.87 (m, 3H), 0.68 (s, 3H) ppm. MS: M/e 503 (M + 1)⁺. |
| | b]pyridin-2-yl)acetonitrile | separated isomer | A199b (the later peak): ¹H NMR (400 MHz, CD₃OD) δ 7.92 (d, J = 4.3 Hz, 1H), 7.32 |
| | | A199b | (d, J = 4.3 Hz, 1H), 6.70 (s, 2H), 5.53 (s, 1H), 5.47 (d, J = 4.2 Hz, 2H), 4.33 (s, 1H), |
| | | (0.2 mg) | 3.82 (d, J = 3.9 Hz, 3H), 3.48 (s, 1H), 3.43 (d, J = 4.2 Hz, 4H), 2.93 (s, 2H), 2.78 (s, |
| | | | 1H), 2.50 (s, 1H), 1.80 (s, 1H), 1.53 (d, J = 53.8 Hz, 4H), 0.95 (m, 7H), 0.70 (s, 6H) |
| | | | ppm. MS: M/e 503 (M + 1)⁺. |
| A201 | 2-(7-((2S,5R)-4-(1-(2,4-difluoro-6- | separated isomer | A201a (the earlier peak): ¹H NMR (400 MHz, CD₃OD) δ 7.91 (s, 1H), 6.68-6.65 (d, J = |
| | methoxyphenyl)ethyl)-2,5- | A201a | 10.8 Hz, 1H), 6.53-6.51 (t, J = 10.1 Hz, 1H), 5.53 (s, 1H), 5.46 (s, 2H), 4.38 (m, 1H), |
| | diethylpiperazin-1-yl)-4-methyl-5- | (1.4 mg) | 3.84 (s, 3H), 3.43 (s, 3H), 3.28-3.16 (m, 2H), 2.98-2.93 (m, 1H), 2.90-2.65 (m, 2H), |
| | oxo-4,5-dihydro-2H-pyrazolo[4,3- | | 2.37-2.35 (d, J = 8.3 Hz, 1H), 2.03-1.92 (m, 1H), 1.82-1.71 (m, 1H), 1.57-1.46 (m, 2H), |
| | b]pyridin-2-yl)acetonitrile | | 1.46-1.44 (d, J = 6.8 Hz, 3H), 0.97-0.93 (t, J = 7.5 Hz, 3H), 0.76-0.72 (t, J = 7.3 Hz, |
| | | | 3H) ppm. MS: M/e 499 (M + 1)⁺. |
| | | separated isomer | A201b (the later peak): ¹H NMR (400 MHz, CD₃OD) δ 7.92 (s, 1H), 6.64-6.62 (d, J = |
| | | A201b | 10.8 Hz, 1H), 6.52-6.46 (m, 1H), 5.52 (s, 1H), 5.47 (s, 2H), 4.32-4.26 (m, 1H), 3.84 (s, |
| | | (0.9 mg) | 3H), 3.50-3.32 (m, 5H), 3.07-3.02 (m, 1H), 2.85-2.58 (m, 2H), 2.39-2.36 (d, J = 12.0 |
| | | | Hz, 1H), 1.96-1.92 (m, 1H), 1.65-1.45 (m, 3H), 1.43-1.41 (d, J = 6.8 Hz, 3H), 1.01- |
| | | | 0.97 (t, J = 7.4 Hz, 3H), 0.63-0.59 (t, J = 7.4 Hz, 3H) ppm. MS: M/e 499 (M + 1)⁺. |
| A203 | 2-(7-((2S,5R)-2,5-diethyl-4-(1-(1- | separated isomer | A203a: ¹H NMR (400 MHz, CDCl₃) δ 8.15 (s, 1H), 7.67 (s, 1H), 7.56-7.51 (m, 1H), |
| | ethyl-5-(trifluoromethyl)-1H- | A203a | 7.40-7.36 (m, 1H), 5.58 (s, 1H), 5.13 (s, 2H), 4.80 (s, 1H), 4.34-4.31 (m, 3H), 3.54- |
| | benzo[d]imidazol-2- | (34 mg, 36%) | 3.50 (m, 1H), 3.42 (s, 3H), 3.03 (s, 1H), 2.81 (s, 1H), 2.43-2.39 (m, 1H), 1.82-1.63 (m, |
| | yl)ethyl)piperazin-1-yl)-4-methyl-5- | | 8H), 1.49-1.45 (m, 3H), 0.99-0.96 (m, 3H), 0.64-0.60 (m, 3H) ppm. MS: M/e 569 |
| | oxo-4,5-dihydro-2H-pyrazolo[4,3- | | (M + 1)⁺. |
| | b]pyridin-2-yl)acetonitrile | separated isomer | A203b: ¹H NMR (400 MHz, CDCl₃) δ 8.05 (s, 1H), 7.56-7.54 (m, 1H), 7.48-7.46 (m, |
| | | A203b | 1H), 7.39 (s, 1H), 5.62 (s, 1H), 5.13 (s, 2H), 4.63-4.50 (m, 1H), 4.48-4.44 (m, 3H), 3.53 |
| | | (48 mg, 51%) | (s, 3H), 3.37-3.33 (m, 1H), 2.96 (s, 2H), 2.49 (s, 1H), 1.80-1.76 (m, 2H), 1.56-1.52 (m, |
| | | | 6H), 1.52-1.48 (m, 3H), 0.75-0.72 (m, 6H) ppm. MS: M/e 569 (M + 1)⁺. |
| A204 | 2-(7-((2S,5R)-2,5-diethyl-4-(1-(1- | separated isomer | Compound A204a: ¹H NMR (400 MHz, CDCl₃) δ 7.85 (d, J = 8 Hz, 1H), 7.66 (s, 1H), |
| | ethyl-6-(trifluoromethyl)-1H- | A204a | 7.54 (d, J = 8 Hz, 1H), 7.40 (s, 1H), 5.60 (s, 1H), 5.13-5.09 (m, 2H), 4.81 (s, 1H), 4.36- |
| | benzo[d]imidazol-2- | (60 mg, 42%) | 4.34 (m, 3H), 3.55-3.51 (m, 1H), 3.42 (s, 3H), 3.04 (s, 1H), 2.84 (s, 1H), 2.41 (s, 1H), |
| | yl)ethyl)piperazin-1-yl)-4-methyl-5- | | 1.76-1.51 (m, 8H), 1.49-1.45 (m, 3H), 0.98-0.94 (m, 3H), 0.65-0.63 (m, 3H) ppm. MS: |
| | oxo-4,5-dihydro-2H-pyrazolo[4,3- | | M/e 569 (M + 1)⁺. |
| | b]pyridin-2-yl)acetonitrile | separated isomer | A204b: ¹H NMR (400 MHz, CDCl₃) δ 7.84 (d, J = 8 Hz, 1H), 7.67 (s, 1H), 7.53 (d, J = |
| | | A204b | 12 Hz, 1H), 7.39 (s, 1H), 5.62 (s, 1H), 5.13 (s, 2H), 4.67-4.63 (m, 1H), 4.50-4.43 (m, |
| | | (64 mg, 46%) | 3H), 3.44 (s, 3H), 3.36-3.33 (m, 1H), 2.97 (s, 2H), 2.48 (s, 1H), 1.78-1.65 (m, 8H), 1.54 |
| | | | (t, J = 8 Hz, 3H), 0.77-0.71 (m, 6H) ppm. MS: M/e 569 (M + 1)⁺. |
| A207 | 2-(7-((2S,5R)-2,5-diethyl-4-(1-(1- | 8 mg, 17% | ¹H NMR (400 MHz, CDCl₃) δ 7.41 (d, J = 4 Hz, 1H), 7.31-2.27 (m, 1H), 6.52-6.47 (m, |
| | ethyl-6-oxo-1,6-dihydropyridin-2- | | 2H), 5.64-5.62 (m, 1H), 5.14 (s, 2H), 4.51-4.30 (m, 2H), 3.93-3.72 (m, 2H), 3.42 (s, |
| | yl)ethyl)piperazin-1-yl)-4-methyl-5- | | 3H), 3.30-3.27 (m, 1H), 3.03-2.98 (m, 0.5H), 2.85 (s, 1H), 2.78-2.73 (m, 0.5H), 2.62- |
| | oxo-4,5-dihydro-2H-pyrazolo[4,3- | | 2.46 (m, 1H), 1.91 (s, 1H), 1.68-1.63 (m, 2H), 1.36-1.32 (m, 3H), 1.32-1.27 (m, 5H), |
| | b]pyridin-2-yl)acetonitrile | | 1.00-0.96 (m, 1H), 0.84-0.80 (m, 4H), 0.73-0.69 (m, 1H) ppm. MS: M/e 478 (M + 1)⁺. |
| A208 | 2-(7-((2S,5R)-2,5-diethyl-4-(1-(1- | A208 | ¹H NMR (400 MHz, CD₃OD) δ 8.43 (s, 1H, HCOOH salt), 7.94 (s, 1H), 7.30 (s, 1H), |
| | ethyl-2-methyl-1H-imidazol-4- | (2 mg, 4%) | 5.55 (d, J = 4.9 Hz, 1H), 5.48 (d, J = 2.2 Hz, 2H), 4.32 (s, 1H), 4.07 (dq, J = 11.4, 7.2 |
| | yl)ethyl)piperazin-1-yl)-4-methyl-5- | | Hz, 3H), 3.87 (q, J = 6.5 Hz, 1H), 3.56-3.38 (m, 4H), 3.05 (dd, J = 20.2, 7.8 Hz, 1H), |
| | oxo-4,5-dihydro-2H-pyrazolo[4,3- | | 2.88-2.69 (m, 2H), 2.54 (d, J = 2.9 Hz, 3H), 1.97-1.52 (m, 4H), 1.50-1.39 (m, 6H), |
| | b]pyridin-2-yl)acetonitrile | | 1.04-0.71 (m, 6H) ppm. MS: M/e 465 (M + 1)⁺. |
| A210 | 2-(7-((2S,5R)-2,5-diethyl-4-(1-(1- | 5 mg, 10% | ¹H NMR (400 MHz, CD₃OD) δ 7.93 (s, 1H), 6.58-6.50 (m, 1H), 5.59-5.50 (m, 1H), |
| | ethyl-3-(trifluoromethyl)-1H- | | 5.49-5.40 (m, 2H), 4.49-3.95 (m, 4H), 3.56-3.34 (m, 4H), 3.10-2.30 (m, 4H), |
| | pyrazol-5-yl)ethyl)piperazin-1-yl)- | | 1.98-1.52 (m, 4H), 1.51-1.26 (m, 6H), 1.05-0.66 (m, 6H) ppm. MS: M/e 519 |
| | 4-methyl-5-oxo-4,5-dihydro-2H- | | (M + 1)⁺. |
| | pyrazolo[4,3-b]pyridin-2- | | |
| | yl)acetonitrile | | |
| A211 | 2-(7-((2S,5R)-2,5-diethyl-4-(1-(1- | 10 mg, 21% | Compound A211: ¹H NMR (400 MHz, CD₃OD) δ 7.93 (s, 1H), 6.78-6.60 (m, 1H), |
| | ethyl-3-(trifluoromethyl)-1H- | | 5.54 (s, 1H), 5.47 (s, 2H), 4.49-4.20 (m, 3H), 4.10-3.75 (m, 1H), 3.52-3.34 (m, |
| | pyrazol-5-yl)ethyl)piperazin-1-yl)- | | 4H), 3.20-2.35 (m, 4H), 1.98-1.52 (m, 4H), 1.50-1.28 (m, 6H), 1.05-0.60 (m, 6H) |
| | 4-methyl-5-oxo-4,5-dihydro-2H- | | ppm. MS: M/e 519 (M + 1)⁺. |
| | pyrazolo[4,3-b]pyridin-2- | | |
| | yl)acetonitrile | | |
| A212 | 2-(7-((2S,5R)-4-(1-(3-chloro-1- | 17 mg, 21% | ¹H NMR (400 MHz, DMSO-d6) δ 7.92 (s, 1H), 6.16 (d, J = 14.1 Hz, 1H), 5.55 (d, J = |
| | ethyl-1H-pyrazol-5-yl)ethyl)-2,5- | | 2.7 Hz, 2H), 5.32 (d, J = 10.7 Hz, 1H), 4.20-3.94 (m, 3H), 3.25 (s, 3H), 3.20 (s, 3H), |
| | diethylpiperazin-1-yl)-4-methyl-5- | | 2.86-2.47 (m, 2H), 2.36-2.24 (m, 1H), 1.75-1.60 (m, 1H), 1.50-1.32 (m, 2.5H), |
| | oxo-4,5-dihydro-2H-pyrazolo[4,3- | | 1.32-1.20 (m, 6.5H), 0.83 (t, J = 7.6 Hz, 1.5H), 0.69-0.58 (m, 4.5H) ppm. MS: M/e 485 |
| | b]pyridin-2-yl)acetonitrile | | (M + 1)+. |
| A213 | 2-(7-((2S,5R)-2,5-diethyl-4-(1-(5- | A213a | A213a (the earlier peak): ¹H NMR (400 MHz, DMSO-d6) δ 7.93 (s, 1H), 7.19 (s, 1H), |
| | ethyl-1-methyl-1H-pyrazol-4- | (10 mg) | 5.57 (s, 2H), 5.33 (s, 1H), 3.69 (s, 3H), 3.61 (q, J = 6.4 Hz, 1H), 3.28 (s, 3H), 3.22 (s, |
| | yl)ethyl)piperazin-1-yl)-4-methyl-5- | | 2H), 3.08 (d, J = 12.5 Hz, 1H), 2.79-2.57 (m, 4H), 2.44-2.38 (m, 1H), 1.91-1.76 (m, |
| | oxo-4,5-dihydro-2H-pyrazolo[4,3- | | 1H), 1.60-1.46 (m, 1H), 1.40-1.27 (m, 2H), 1.23 (d, J = 6.5 Hz, 3H), 1.07 (t, J = 7.5 Hz, |
| | b]pyridin-2-yl)acetonitrile | | 3H), 0.80 (t, J = 7.2 Hz, 3H), 0.62 (t, J = 7.2 Hz, 3H) ppm. MS: M/e 465 (M + 1)⁺ |
| | | A213b | A213b (the later peak): ¹H NMR (400 MHz, DMSO-d6) δ 7.91 (s, 1H), 7.15 (s, 1H), |
| | | (12 mg) | 5.55 (s, 2H), 5.29 (s, 1H), 3.65 (s, 3H), 3.48 (q, J = 6.6 Hz, 1H), 3.25 (s, 3H), 3.20 (s, |
| | | | 1H), 3.20 (s, 2H), 2.88 (s, 1H), 2.69-2.59 (m, 1H), 2.59-2.49 (m, 1H), 2.41-2.37 (m, |
| | | | 2H), 1.79-1.66 (m, 1H), 1.47-1.33 (m, 3H), 1.16 (d, J = 6.4 Hz, 3H), 1.01 (t, J = 7.5 |
| | | | Hz, 3H), 0.84 (t, J = 7.3 Hz, 3H), 0.55 (t, J = 7.2 Hz, 3H) ppm. MS: M/e 465 (M + 1)⁺. |
| A214 | 2-(7-((2S,5R)-2,5-diethyl-4-(1-(1- | 2 mg, 17% | ¹H NMR (400 MHz, CD₃OD δ 7.94 (d, J = 7.0 Hz, 1H), 7.79-7.63 (m, 2H), 7.51- |
| | (2-hydroxyethyl)-1H- | | 7.34 (m, 2H), 5.55 (s, 1H), 5.47 (d, J = 5.4 Hz, 2H), 4.64 (dd, J = 18.4, 13.2 Hz, 3H), |
| | benzo[d]imidazol-2- | | 4.00-3.92 (m, 2H), 3.61 (d, J = 16.0 Hz, 1H), 3.56-3.46 (m, 1H), 3.43 (d, J = 3.1 |
| | yl)ethyl)piperazin-1-yl)-4-methyl-5- | | Hz, 3H), 3.18 (dd, J = 22.7, 15.4 Hz, 1H), 3.01 (s, 1H), 2.78 (s, 1H), 2.49 (s, 1H), 1.98- |
| | oxo-4,5-dihydro-2H-pyrazolo[4,3- | | 1.78 (m, 1H), 1.77-1.53 (m, 6H), 1.30 (d, J = 7.1 Hz, 3H), 0.78 (dd, J = 13.0, 5.6 |
| | b]pyridin-2-yl)acetonitrile | | Hz, 3H) ppm. MS: M/e 517 (M + 1)⁺. |
| A215 | 2-(7-((2S,5R)-2,5-diethyl-4-(1-(1- | separated isomer | A215a (the earlier peak): ¹H NMR (400 MHz, CDCl₃) δ 8.57 (s, 1H), 7.73 (s, 1H), 7.41 |
| | ethyl-1H-imidazo[4,5-b]pyridin-2- | A215a | (s, 1H), 7.27 (s, 1H), 5.60 (s, 1H), 5.16-5.15 (m, 2H), 4.77-4.73 (m, 1H), 4.47-4.33 (m, |
| | yl)ethyl)piperazin-1-yl)-4-methyl-5- | (2 mg) | 3H), 3.59-3.54 (m, 2H), 3.42 (s, 3H), 3.04-2.85 (m, 2H), 2.50-2.43 (m, 1H), 2.03-1.57 |
| | oxo-4,5-dihydro-2H-pyrazolo[4,3- | | (m, 7H), 1.50-1.46 (m, 3H), 0.95 (s, 3H), 0.68-0.63 (m, 3H) ppm. MS: M/e 502 (M + 1)⁺. |
| | b]pyridin-2-yl)acetonitrile | separated isomer | A215b (the later peak): ¹H NMR (400 MHz, CDCl₃) δ 8.54 (s, 1H), 7.73-7.71 (m, 1H), |
| | | A215b | 7.39 (s, 1H), 7.25 (s, 1H), 5.62 (s, 1H), 5.13 (s, 2H), 4.60-4.56 (m, 1H), 4.47-4.46 (m, |
| | | (2 mg) | 3H), 3.56-3.52 (m, 1H), 3.43-3.39 (m, 4H), 3.02-2.98 (m, 2H), 2.55-2.51 (m, 1H), 2.03- |
| | | | 1.66 (m, 7H), 1.51-1.49 (m, 3H), 0.76-0.73 (m, 6H) ppm. MS: M/e 502 (M + 1)⁺. |
| A216 | 2-(7-((2S,5R)-2,5-diethyl-4-(1-(1- | 2 mg, 4% | ¹H NMR (400 MHz, CD₃OD) δ 7.92 (s, 1H), 7.38-7.30 (m, 1H), 5.59-5.50 (m, 1H), |
| | ethyl-4-methyl-1H-pyrazol-3- | | 5.49-5.40 (m, 2H), 4.14-3.98 (m, 2H), 3.98-3.75 (m, 1H), 3.51-3.34 (m, 4H), |
| | yl)ethyl)piperazin-1-yl)-4-methyl-5- | | 3.20-2.30 (m, 4H), 2.14 (s, 3H), 2.10-1.68 (m, 2H), 1.60-1.46 (m, 3H), 1.45-1.30 |
| | oxo-4,5-dihydro-2H-pyrazolo[4,3- | | (m, 6H), 1.02-0.80 (m, 3H), 0.75-0.56 (m, 3H) ppm. MS: M/e 465 (M + 1)⁺. |
| | b]pyridin-2-yl)acetonitrile | | |
| A217 | 2-(7-((2S,5R)-2,5-diethyl-4-(1-(2- | 8 mg, 15% | ¹H NMR (400 MHz, CD₃OD) δ 8.57-8.56 (m, 1H), 8.49-8.47 (m, 1H), 7.95 (s, 1H), |
| | (trifluoromethyl)pyridin-3- | | 7.71 (s, 1H), 5.58 (s, 1H), 5.48 (s, 2H), 4.27-4.09 (m, 1H), 3.52-3.48 (m, 1H), 3.44 |
| | yl)ethyl)piperazin-1-yl)-4-methyl-5- | | (s, 3H), 3.21-3.13 (m, 1H), 2.92-2.67 (m, 2H), 2.29-2.03 (m, 2H), 1.88-1.49 (m, |
| | oxo-4,5-dihydro-2H-pyrazolo[4,3- | | 4H), 1.35-1.31 (m, 3H), 1.08-1.02 (m, 3H), 0.74-0.58 (m, 3H) ppm. MS: M/e 502 |
| | b]pyridin-2-yl)acetonitrile | | (M + 1)⁺. |
| A219 | 2-(7-((2S,5R)-2,5-diethyl-4-(1-(1- | 2 mg, 4% | ¹H NMR (400 MHz, CD₃OD) δ 8.45 (s, 1H), 7.94 (d, J = 3.4 Hz, 1H), 7.14 (d, J = 21.3 |
| | ethyl-2-methyl-1H-imidazol-5- | | Hz, 1H), 5.56 (s, 1H), 5.48 (d, J = 7.2 Hz, 2H), 4.36-4.07 (m, 4H), 3.59-3.38 (m, |
| | yl)ethyl)piperazin-1-yl)-4-methyl-5- | | 4H), 2.99-2.59 (m, 3H), 2.54 (d, J = 1.0 Hz, 3H), 1.81-1.59 (m, 3H), 1.52-1.30 (m, |
| | oxo-4,5-dihydro-2H-pyrazolo[4,3- | | 8H), 1.00 (t, J = 7.3 Hz, 1H), 0.88-0.74 (m, 5H) ppm. MS: M/e 465 (M + 1)⁺. |
| | b]pyridin-2-yl)acetonitrile | | |
| A221 | 2-ethyl-7-((2S,5R)-5-ethyl-2- | separated isomer | A221a (the earlier peak): ¹H NMR (400 MHz, CD₃OD) δ 8.88 (d, J = 3.2 Hz, 2H), 8.12 |
| | methyl-4-(1-(quinoxalin-6- | A221a | (d, J = 8.7 Hz, 1H), 8.08-8.00 (m, 2H), 7.81 (s, 1H), 5.55 (s, 1H), 4.71 (s, 1H), 4.28 |
| | yl)ethyl)piperazin-1-yl)-4-methyl- | (4 mg) | (q, J = 7.3 Hz, 2H), 4.07 (q, J = 6.4 Hz, 1H), 3.43 (s, 3H), 3.35 (d, J = 3.0 Hz, 2H), 3.03 |
| | 2,4-dihydro-5H-pyrazolo[4,3- | | (dd, J = 11.9, 3.7 Hz, 1H), 2.92 (d, J = 10.0 Hz, 1H), 2.43 (s, 1H), 1.62 (dd, J = 14.6, |
| | b]pyridin-5-one | | 7.2 Hz, 2H), 1.53-1.40 (m, 9H), 0.71 (t, J = 7.4 Hz, 3H) ppm. MS: M/e 460 (M + 1)⁺. |
| | | separated isomer | A221b (the later peak): ¹H NMR (400 MHz, CD₃OD) δ 8.90-8.83 (m, 2H), 8.06 (dd, |
| | | A221b | J = 16.2, 7.1 Hz, 3H), 7.83 (s, 1H), 5.53 (s, 1H), 5.14 (s, 1H), 4.43 (s, 1H), 4.31 (q, J = |
| | | (5 mg) | 7.3 Hz, 2H), 3.93 (q, J = 6.6 Hz, 1H), 3.58 (d, J = 11.0 Hz, 1H), 3.46 (d, J = 15.9 Hz, |
| | | | 3H), 3.22 (d, J = 9.9 Hz, 1H), 2.82 (dd, J = 12.0, 4.1 Hz, 1H), 2.25 (d, J = 11.7 Hz, 1H), |
| | | | 1.82-1.70 (m, 1H), 1.62 (dd, J = 13.8, 7.3 Hz, 1H), 1.52 (t, J = 7.3 Hz, 3H), 1.45 (d, J = |
| | | | 6.6 Hz, 3H), 1.23 (d, J = 6.6 Hz, 3H), 1.08 (t, J = 7.3 Hz, 3H) ppm. MS: M/e 460 |
| | | | (M + 1)⁺. |
| A222 | 2-(2,2-difluoroethyl)-7-((2S,5R)-5- | separated isomer | A222a (the earlier peak): ¹H NMR (400 MHz, CD₃OD) δ 8.88 (dd, J = 5.0, 1.8 Hz, |
| | ethyl-2-methyl-4-(1-(quinoxalin-6- | A222a | 2H), 8.12 (d, J = 8.6 Hz, 1H), 8.08-7.99 (m, 2H), 7.88 (s, 1H), 6.40-6.03 (m, 1H), |
| | yl)ethyl)piperazin-1-yl)-4-methyl- | (6 mg) | 5.58 (s, 1H), 4.67 (td, J = 14.3, 3.7 Hz, 3H), 4.08 (q, J = 6.5 Hz, 1H), 3.44 (s, 3H), 3.36 |
| | 2,4-dihydro-5H-pyrazolo[4,3- | | (d, J = 3.5 Hz, 2H), 3.03 (dd, J = 11.6, 3.9 Hz, 1H), 2.93 (d, J = 11.8 Hz, 1H), 2.44 (s, |
| | b]pyridin-5-one | | 1H), 1.68-1.58 (m, 2H), 1.45 (t, J = 6.5 Hz, 6H), 0.70 (t, J = 7.4 Hz, 3H) ppm. MS: |
| | | | M/e 496 (M + 1)⁺. |
| | | separated isomer | A222b (the later peak): ¹H NMR (400 MHz, CD₃OD) δ 8.87 (dd, J = 6.1, 1.8 Hz, 2H), |
| | | A222b | 8.06 (dd, J = 16.6, 7.0 Hz, 3H), 7.89 (s, 1H), 6.22 (td, J = 55.1, 27.7 Hz, 1H), 5.56 (s, |
| | | (5 mg). | 1H), 5.03 (s, 1H), 4.70 (td, J = 14.4, 3.3 Hz, 2H), 4.45 (s, 1H), 3.93 (q, J = 6.6 Hz, 1H), |
| | | | 3.59 (d, J = 11.4 Hz, 1H), 3.44 (s, 3H), 3.22 (d, J = 10.3 Hz, 1H), 2.82 (dd, J = 12.0, |
| | | | 4.0 Hz, 1H), 2.25 (d, J = 12.0 Hz, 1H), 1.83-1.70 (m, 1H), 1.62 (dd, J = 13.3, 7.2 Hz, |
| | | | 1H), 1.45 (d, J = 6.6 Hz, 3H), 1.22 (d, J = 6.5 Hz, 3H), 1.06 (t, J = 7.4 Hz, 3H) ppm. |
| | | | MS: M/e 496 (M + 1)⁺. |
| A223 | 2-(7-((2S,5R)-4-(1- | 1 mg, 11% for two | ¹HNMR (400 MHz, CD₃OD) δ 7.93 (s, 1H), 5.57 (s, 1H), 5.49 (s, 2H), 4.82-4.66 (m, |
| | cyclohexylethyl)-2,5- | steps | 0.5H), 4.50-4.30 (m, 0.5H), 3.66-3.50 (m, 1H), 3.44 (s, 3H), 3.20-3.04 (m, 2H), |
| | dimethylpiperazin-1-yl)-4-methyl-5- | | 2.86-2.65 (m, 0.5H), 2.56-2.30 (m, 2H), 2.07-1.95 (m, 1.5H), 1.81-1.64 (m, 4H), |
| | oxo-4,5-dihydro-2H-pyrazolo[4,3- | | 1.41-0.95 (m, 15H) ppm. MS: M/e 411 (M + 1)⁺. |
| | b]pyridin-2-yl)acetonitrile | | |
| A226 | 2-(7-((2S,5R)-4-(1-(6-chloro-4- | 6 mg, 11% | ¹H NMR (400 MHz, CD₃OD) δ 9.02 (d, J = 8 Hz, 1H), 7.93 (s, 1H), 7.72 (s, 1H), 5.58 |
| | (trifluoromethyl)pyridin-3-yl)ethyl)- | | (s, 1H), 5.47 (s, 2H), 4.25-4.04 (m, 1H), 3.56-3.52 (m, 1H), 3.44 (s, 3H), 3.30-3.10 (m, |
| | 2,5-diethylpiperazin-1-yl)-4-methyl- | | 1H), 2.78-2.32 (m, 2H), 2.17-2.14 (m, 2H), 1.75-1.71 (m, 1H), 1.71-1.68 (m, 2H), 1.52- |
| | 5-oxo-4,5-dihydro-2H-pyrazolo[4,3- | | 1.46 (m, 1H), 1.37-1.32 (m, 3H), 1.04-0.99 (m, 3H), 0.77-0.60 (m, 3H) ppm. MS: M/e |
| | b]pyridin-2-yl)acetonitrile. | | 536 (M + 1)⁺. |
| A227 | 2-(7-((2S,5R)-2,5-diethyl-4-(1-(1- | separated isomer | Compound A227a (the earlier peak): ¹H NMR (400 MHz, CD₃OD) δ 7.93 (s, 1H), |
| | ethyl-4-methyl-1H-pyrazol-5- | A227a | 7.24(s, 1H), 5.56 (s, 1H), 5.47 (s, 2H), 4.54-3.45 (m, 1H), 4.25-3.85 (m, 2H), 3.51- |
| | yl)ethyl)piperazin-1-yl)-4-methyl-5- | (1 mg) | 3.34 (m, 4H), 3.20-2.15 (m, 1H), 2.80-2.55 (m, 3H), 2.30-2.20 (m, 1H), 2.14 (s, |
| | oxo-4,5-dihydro-2H-pyrazolo[4,3- | | 3H), 2.02-1.50 (m, 4H), 1.50-1.30 (m, 6H), 1.08-0.95 (m, 3H), 0.75-0.56 (m, 3H) |
| | b]pyridin-2-yl)acetonitrile | | ppm. MS: M/e 465 (M + 1)⁺. |
| | | separated isomer | A227b (the later peak): ¹H NMR (400 MHz, CD₃OD) δ 7.93 (s, 1H), 7.27(s, 1H), 5.58 |
| | | A227b | (s, 1H), 5.47 (s, 2H), 4.50-4.25 (m, 2H), 4.15-3.98 (m, 1H), 3.42 (s, 3H), 3.10-3.00 |
| | | (1 mg) | (m, 1H), 2.90-2.60 (m, 4H), 2.30-2.20 (m, 1H), 2.09-2.00 (m, 4H), 1.90-1.50 (m, |
| | | | 2H), 1.50-1.30 (m, 7H), 1.08-0.85 (m, 3H), 0.75-0.56 (m, 3H) ppm. MS: M/e 465 |
| | | | (M + 1)⁺. |
| A228 | 7-((2S,5R)-4-(1-(1-(2,2- | 8 mg, 37% | Compound A228: ¹H NMR (400 MHz, CD₃OD) δ 7.93 (d, J = 3.0 Hz, 1H), 7.50 (d, J = |
| | difluoroethyl)-1H-pyrazol-5- | | 3.5 Hz, 1H), 6.42-6.14 (m, 2H), 5.55 (d, J = 14.7 Hz, 1H), 5.48 (d, J = 4.7 Hz, 2H), |
| | yl)ethyl)-2,5-diethylpiperazin-1-yl)- | | 4.79-4.64 (m, 2H), 4.19 (d, J = 7.0 Hz, 1H), 4.09 (d, J = 6.5 Hz, 1H), 3.43 (s, 4H), |
| | 4-methyl-2,4-dihydro-5H- | | 1.91-1.64 (m, 3H), 1.46 (d, J = 6.7 Hz, 2H), 1.41 (d, J = 6.7 Hz, 2H), 1.07-0.82 (m, |
| | pyrazolo[4,3-b]pyridin-5-one | | 5H), 0.82-0.72 (m, 5H) ppm. MS: M/e 487 (M + 1)⁺ |
| A230 | 2-(7-((2S,5R)-2,5-diethyl-4-(1-(1- | separated isomer | A230a (the earlier peak): ¹H NMR (400 MHz, CDCl₃) δ 7.97 (d, J = 8 Hz, 1H), 7.63(d, |
| | ethyl-7-(trifluoromethyl)-1H- | A230a | J = 8 Hz, 1H), 7.37 (s, 1H), 7.33-7.28 (m, 1H), 5.58 (s, 1H), 5.14 (s, 2H), 4.70-4.32 (m, |
| | benzo[d]imidazol-2- | (5 mg, 8%) | 4H), 3.54-3.50 (m, 1H), 3.42 (s, 3H), 3.01-2.82 (m, 2H), 2.48-2.44 (m, 1H), 1.87-1.82 |
| | yl)ethyl)piperazin-1-yl)-4-methyl-5- | | (m, 2H), 1.66-1.61 (m, 6H), 1.39-1.37 (m, 3H), 0.97-0.95 (m, 3H), 0.69-0.64 (m, 3H) |
| | oxo-4,5-dihydro-2H-pyrazolo[4,3- | | ppm. MS: M/e 569 (M + 1)⁺. |
| | b]pyridin-2-yl)acetonitrile | separated isomer | Compound A230b(the later peak): ¹H NMR (400 MHz, CDCl₃) δ 7.96 (d, J = 8 Hz, |
| | | A230b | 1H), 7.61 (d, J = 4 Hz, 1H), 7.37 (s, 1H), 7.33-7.29 (m, 1H), 5.59 (s, 1H), 5.12 (s, 2H), |
| | | (8 mg, 13%) | 4.60-4.56 (m, 2H), 4.35-4.33 (m, 1H), 3.40-3.37 (m, 4H), 3.06-3.04 (m, 1H), 2.79-2.76 |
| | | | (m, 2H), 1.79-1.75 (m, 1H), 1.72-1.70 (m, 4H), 1.58-1.56 (m, 4H), 1.44-1.40 (m, 3H), |
| | | | 0.83 (t, J = 8 Hz, 3H), 0.47-0.43 (m, 3H) ppm. MS: M/e 569 (M + 1)⁺. |
| A234 | 2-(7-((2S,5R)-4-(1-(5- | 6 mg, 9% | ¹H NMR (400 MHz, CD₃OD) δ 7.93 (s, 1H), 7.47 (s, 1H), 7.27 (dt, J = 52.6, 26.2 Hz, |
| | (difluoromethyl)-1-methyl-1H- | | 1H), 5.55 (d, J = 4.6 Hz, 1H), 5.47 (d, J = 2.3 Hz, 2H), 3.99 (d, J = 7.3 Hz, 3H), 3.43 |
| | pyrazol-4-yl)ethyl)-2,5- | | (s, 4H), 3.16-2.45 (m, 4H), 2.05-1.74 (m, 2H), 1.70-1.45 (m, 3H), 1.34 (dd, J = |
| | diethylpiperazin-1-yl)-4-methyl-5- | | 20.3, 14.0 Hz, 4H), 0.97 (dt, J = 22.9, 7.3 Hz, 3H), 0.72 (dd, J = 18.6, 11.4 Hz, 3H) |
| | oxo-4,5-dihydro-2H-pyrazolo[4,3- | | ppm. MS: M/e 487 (M + 1)⁺. |
| | b]pyridin-2-yl)acetonitrile | | |
| A235 | 2-(7-((2S,5R)-2,5-diethyl-4-(1-(6- | 19 mg, 35% | ¹H NMR (400 MHz, CDCl₃) δ 9.04 (s, 1H), 7.93 (s, 1H), 7.55 (d, J = 4 Hz, 1H), 5.58 |
| | methyl-4-(trifluoromethyl)pyridin- | | (s, 1H), 5.47-5.46 (m, 1H), 4.24-4.03 (m, 1H), 3.54-3.50 (m, 0.5H), 3.42 (s, 3H), 3.30- |
| | 3-yl)ethyl)piperazin-1-yl)-4-methyl- | | 3.28 (m, 2.5H), 3.13-3.09 (m, 0.5H), 2.92-2.78 (m, 1H), 2.62 (s, 3H), 2.31-2.27 (m, |
| | 5-oxo-4,5-dihydro-2H-pyrazolo[4,3- | | 0.5H), 2.17-2.13 (m, 1H), 1.92-1.88(m, 1H), 1.66-1.61 (m, 2H), 1.53-1.49 (m, 1H), |
| | b]pyridin-2-yl)acetonitrile | | 1.38-1.34 (m, 3H), 1.28-1.24 (m, 1H), 1.09-1.05 (m, 3H), 0.76-0.58 (m, 3H) ppm. MS: |
| | | | M/e 516 (M + 1)⁺. |
| A237 | 2-(7-((2S,5R)-4-(1-(1-(2,2- | 6 mg, 26% | ¹H NMR (400 MHz, CD₃OD) δ 7.93 (d, J = 4.1 Hz, 1H), 7.69 (d, J = 5.9 Hz, 1H), 6.94 |
| | difluoroethyl)-1H-imidazol-5- | | (d, J = 21.1 Hz, 1H), 5.48 (t, J = 6.5 Hz, 3H), 4.77-4.55 (m, 2H), 4.26-3.95 (m, 2H), |
| | yl)ethyl)-2,5-diethylpiperazin-1-yl)- | | 3.57-3.47 (m, 1H), 3.43 (s, 3H), 2.94-2.58 (m, 5H), 1.75-1.59 (m, 3H), 1.45 (dd, |
| | 4-methyl-5-oxo-4,5-dihydro-2H- | | J = 24.7, 6.7 Hz, 4H), 0.99 (t, J = 7.1 Hz, 2H), 0.85-0.72 (m, 4H) ppm. MS: M/e 487 |
| | pyrazolo[4,3-b]pyridin-2- | | (M + 1)+ |
| | yl)acetonitrile | | |
| A239 | 2-(7-((2S,5R)-2,5-diethyl-4-(1-(1- | 50 mg, 2% | ¹H NMR (400 MHz, CD₃OD) δ 7.93 (d, J = 3.8 Hz, 1H), 7.07 (d, J = 12.7 Hz, 1H), 5.53 |
| | ethyl-2-(trifluoromethyl)-1H- | | (d, J = 17.1 Hz, 1H), 5.48 (d, J = 6.0 Hz, 2H), 4.50-4.07 (m, 4H), 3.53 (d, J = 10.6 Hz, |
| | imidazol-5-yl)ethyl)piperazin-1-yl)- | | 1H), 3.43 (s, 3H), 2.98 (d, J = 8.4 Hz, 1H), 2.82-2.71 (m, 2H), 2.57 (dd, J = 11.9, 5.4 |
| | 4-methyl-5-oxo-4,5-dihydro-2H- | | Hz, 1H), 1.72-1.60 (m, 3H), 1.53 (d, J = 6.7 Hz, 2H), 1.45 (s, 2H), 1.41-1.28 (m, 4H), |
| | pyrazolo[4,3-b]pyridin-2- | | 1.01 (t, J = 7.3 Hz, 1H), 0.87 (t, J = 7.4 Hz, 1H), 0.73 (dt, J = 41.2, 7.4 Hz, 3H) ppm. |
| | yl)acetonitrile | | MS: M/e 519 (M + 1)⁺ |
| A241 | 2-(7-((2S,5R)-2,5-diethyl-4-(4- | 8 mg, 15% | ¹H NMR (400 MHz, DMSO) δ 8.06 (s, 1H), 7.98-7.88 (m, 1H), 7.70-7.59 (m, 2H), |
| | fluoro-2- | | 5.69 (s, 2H), 5.49 (s, 1H), 4.62-4.29 (m, 1H), 3.89 (d, J = 14.5 Hz, 1H), 3.77 (d, J = |
| | (trifluoromethyl)benzyl)piperazin-1- | | 14.7 Hz, 1H), 3.40-3.37 (m, 2H), 3.34 (s, 3H), 2.84 (d, J = 8.6 Hz, 1H), 2.71 (d, J = |
| | yl)-4-methyl-5-oxo-4,5-dihydro-2H- | | 10.2 Hz, 1H), 2.46 (d, J = 11.7 Hz, 1H), 1.94 (dt, J = 15.5, 7.9 Hz, 1H), 1.69-1.51 (m, |
| | pyrazolo[4,3-b]pyridin-2- | | 3H), 0.92 (t, J = 7.3 Hz, 3H), 0.74 (t, J = 7.3 Hz, 3H) ppm. MS: M/e 505 (M + 1)⁺. |
| | yl)acetonitrile | | |
| A242 | 2-(7-((2S,5R)-2,5-dimethyl-4-(1- | 6 mg, 54% | ¹HNMR (400 MHz, CD₃OD) δ 7.93-7.91 (m, 1H), 5.58-5.48 (m, 3H), 5.08-4.98 (m, |
| | (tetrahydro-2H-pyran-4- | | 0.5H), 4.82-4.65 (m, 0.5H), 4.52-4.15 (m, 1H), 4.05-3.85 (m, 2H), 3.72-3.52 (m, |
| | yl)ethyl)piperazin-1-yl)-4-methyl-5- | | 1H), 3.44 (s, 3H), 3.43-3.34 (m, 2H), 3.21-3.02 (m, 1.5H), 2.90-2.80 (m, 0.5H), |
| | oxo-4,5-dihydro-2H-pyrazolo[4,3- | | 2.72-2.60 (m, 0.5H), 2.55-2.25 (m, 1.5H), 2.10-1.96 (m, 0.5H), 1.95-1.84 (m, |
| | b]pyridin-2-yl)acetonitrile | | 0.5H), 1.71-1.45 (m, 2H), 1.38-1.26 (m, 4H), 1.22 (d, J = 6.4 Hz, 1H), 1.15 (d, J = |
| | | | 6.8 Hz, 2H), 1.07 (d, J = 7.2 Hz, 2H), 1.00 (t, J = 6.8 Hz, 2H) ppm. MS: M/e 413 |
| | | | (M + 1)⁺. |
| A246 | 2-(7-((2S,5R)-4-(4-fluoro-2- | 21 mg, 20% | ¹H NMR (400 MHz, CD₃OD) δ 7.98-7.91 (m, 2H), 7.46-7.34 (m, 2H), 5.59 (s, 1H), |
| | (trifluoromethyl)benzyl)-2,5- | | 5.48 (s, 2H), 4.76 (s, 1H), 4.47 (s, 1H), 3.80-3.60 (m, 2H), 3.62 (dd, J = 13.1, 3.4 Hz, |
| | dimethylpiperazin-1-yl)-4-methyl-5- | | 1H), 3.44 (s, 3H), 3.17 (s, 1H), 3.00 (dd, J = 11.7, 4.2 Hz, 1H), 2.34 (d, J = 11.8 Hz, |
| | oxo-4,5-dihydro-2H-pyrazolo[4,3- | | 1H), 1.31 (d, J = 6.6 Hz, 3H), 1.15 (d, J = 6.5 Hz, 3H) ppm. MS: M/e 477 (M + 1)⁺. |
| | b]pyridin-2-yl)acetonitrile | | |
| A248 | 2-(7-((2S,5R)-4-(1-(2-omethoxy-4- | 2 mg | ¹H NMR (400 MHz, CD₃OD) δ 7.93 (d, J = 2.4 Hz, 1H), 7.62-7.46 (m, 1H), 6.82- |
| | fluorophenyl)ethyl)-2,5- | | 6.61 (m, 2H), 5.56 (d, J = 3.6 Hz, 1H), 5.48 (d, J = 1.6 Hz, 2H), 4.43-4.14 (m, 1H), |
| | dimethylpiperazin-1-yl)-4-methyl-5- | | 4.09-3.98 (m, 0.5H), 3.82 (d, J = 5.2 Hz, 3H), 3.67-3.52 (m, 1H), 3.43 (s, 3H), 3.40- |
| | oxo-4,5-dihydro-2H-pyrazolo[4,3- | | 3.37 (m, 0.5H), 3.06-2.65 (m, 3.5H), 2.32-2.18 (m, 0.5H), 1.40-1.35 (m, 1.5H), |
| | b]pyridin-2-yl)acetonitrile | | 1.30-1.21 (m, 4.5H), 1.17-0.96 (m, 3H) ppm. MS: M/e 453 (M + 1)⁺. |
| A249 | 2-(7-((2S,5R)-4-(1-(4-fluoro-2- | 15 mg, 46% | ¹H NMR (400 MHz, CD₃OD) δ 7.92 (s, 1H), 7.47-7.33 (m, 1H), 7.21-7.08 (m, 2H), |
| | (trifluoromethoxy)phenyl)ethyl)- | | 5.55 (s, 1H), 5.48 (d, J = 1.6 Hz, 2H), 4.72-4.44 (m, 1H), 4.28-4.08 (m, 1H), 3.66- |
| | 2,5-dimethylpiperazin-1-yl)-4- | | 3.51 (m, 1H), 3.43 (s, 3H), 3.40-3.34 (m, 1H), 3.03-2.74 (m, 2H), 2.11 (d, J = 11.6 |
| | methyl-5-oxo-4,5-dihydro-2H- | | Hz, 1H), 1.50 (dd, J = 18.0, 6.8 Hz, 3H), 1.34 (d, J = 6.4 Hz, 1.5H), 1.13 (dd, J = 14.4, |
| | pyrazolo[4,3-b]pyridin-2- | | 6.4 Hz, 3H), 1.04 (d, J = 6.4 Hz, 1.5H) ppm. MS: M/e 507 (M + 1)⁺. |
| | yl)acetonitrile | | |
| A250 | 2-(7-((2S,5R)-4-(1-(2,3-dihydro- | 8 mg | ¹H NMR (400 MHz, CD₃OD) δ 7.93 (s, 1H), 7.68 (s, 1H), 7.37 (s, 1H), 5.56 (s, 1H), |
| | [1,4]dioxino[2,3-b]pyridin-7- | | 5.48 (s, 2H), 4.69-4.56 (m, 1H), 4.55-4.40 (m, 2H), 4.34-4.22 (m, 2H), 3.69-3.48 |
| | yl)ethyl)-2,5-dimethylpiperazin-1- | | (m, 3H), 3.43 (s, 3H), 3.01-2.74 (m, 2H), 2.23 (d, J = 12.5 Hz, 1H), 1.43-1.27 (m, |
| | yl)-4-methyl-5-oxo-4,5-dihydro-2H- | | 5H), 1.26-1.05 (m, 3H), 0.99 (d, J = 20.7, 6.4 Hz, 1H). MS: M/e 464 (M + 1)⁺. |
| | pyrazolo[4,3-b]pyridin-2- | | |
| | yl)acetonitrile | | |
| A251 | 2-(7-((2S,5R)-4-(1-(4-chloro-2- | 5 mg, 23% | ¹H NMR (400 MHz, CD₃OD) δ 8.05 (dd, J = 17.0, 8.7 Hz, 1H), 7.93 (s, 1H), 7.66 (t, J = |
| | (trifluoromethyl)phenyl)ethyl)-2,5- | | 8.5 Hz, 2H), 5.56 (s, 1H), 5.48 (s, 2H), 4.10-3.91 (m, 1H), 3.69-3.60 (m, 1H), 3.43 |
| | dimethylpiperazin-1-yl)-4-methyl-5- | | (s, 3H), 3.38 (s, 1H), 3.22-2.57 (m, 3H), 1.43-1.25 (m, 5H), 1.19-0.97 (m, 5H). |
| | oxo-4,5-dihydro-2H-pyrazolo[4,3- | | MS: M/e 507 (M + 1)⁺ |
| | b]pyridin-2-yl)acetonitrile | | |
| A252 | 2-(7-((2S,5R)-4-(1-(2- | 12 mg, 37% | ¹H NMR (400 MHz, CD₃OD) δ 7.94 (s, 1H), 7.88-7.44 (m, 2H), 7.30 (d, J = 35.5 Hz, |
| | (difluoromethyl)-4- | | 2H), 5.57 (s, 1H), 5.48 (s, 2H), 3.63 (s, 1H), 3.44 (s, 3H), 2.83 (t, J = 51.0 Hz, 3H), |
| | fluorophenyl)ethyl)-2,5- | | 2.10-1.96 (m, 3H), 1.39 (d, J = 4.7 Hz, 6H), 0.90 (t, J = 6.7 Hz, 3H). MS: M/e 473 |
| | dimethylpiperazin-1-yl)-4-methyl-5- | | (M + 1)⁺ |
| | oxo-4,5-dihydro-2H-pyrazolo[4,3- | | |
| | b]pyridin-2-yl)acetonitrile | | |
| A253 | 2-(7-((2S,5R)-4-(1-(3- | 23 mg, 36% | ¹H NMR (400 MHz, CD₃OD) δ 7.93 (s, 1H), 7.33 (dt, J = 13.4, 7.7 Hz, 1H), 7.19 (dd, |
| | fluorophenyl)ethyl)-2,5- | | J = 18.4, 9.8 Hz, 2H), 7.03-6.90 (m, 1H), 5.56 (s, 1H), 5.48 (d, J = 1.9 Hz, 2H), 4.63 |
| | dimethylpiperazin-1-yl)-4-methyl-5- | | (s, 1H), 3.60 (t, J = 19.8 Hz, 2H), 3.43 (s, 3H), 3.14-2.67 (m, 3H), 2.23 (d, J = 10.8 |
| | oxo-4,5-dihydro-2H-pyrazolo[4,3- | | Hz, 1H), 1.41-1.22 (m, 6H), 1.16-1.00 (m, 3H). MS: M/e 423 (M + 1)⁺. |
| | b]pyridin-2-yl)acetonitrile | | |
| A255 | 2-(7-((2S,5R)-2,5-dimethyl-4-(1- | 12 mg, 37% | ¹H NMR (400 MHz, CD₃OD) δ 8.89 (s, 2H), 8.19-8.02 (m, 2H), 8.00 (d, J = 8.4 Hz, |
| | (quinoxalin-6-yl)propyl)piperazin-1- | | 1H), 7.93 (s, 1H), 5.56 (s, 1H), 5.47 (d, J = 2.1 Hz, 2H), 3.88-3.52 (m, 3H), 3.43 (s, |
| | yl)-4-methyl-5-oxo-4,5-dihydro-2H- | | 3H), 1.95-1.64 (m, 2H), 1.41 (d, J = 7.5 Hz, 2H), 1.22 (d, J = 6.0 Hz, 4H), 1.06 (s, |
| | pyrazolo[4,3-b]pyridin-2- | | 2H), 0.93-0.85 (m, 2H), 0.72 (dd, J = 17.1, 7.6 Hz, 3H) ppm. MS: M/e 471 (M + 1)⁺ |
| | yl)acetonitrile | | |
| A256 | 2-(7-((2S,5R)-4-(1-(4-fluoro-2- | 1.2 mg, 8% | ¹H NMR (400 MHz, CD₃OD) δ 8.03-7.90 (m, 2H), 7.42 (dd, J = 13.6, 5.7 Hz, 2H), |
| | (trifluoromethyl)phenyl)propyl)-2,5- | | 5.55 (s, 1H), 5.47 (s, 2H), 4.06-3.81 (m, 1H), 3.62 (dd, J = 18.7, 15.8 Hz, 1H), 3.49- |
| | dimethylpiperazin-1-yl)-4-methyl-5- | | 3.36 (m, 4H), 3.15-2.88 (m, 1H), 2.83 (d, J = 12.3 Hz, 1H), 2.10-1.99 (m, 1H), 2.00- |
| | oxo-4,5-dihydro-2H-pyrazolo[4,3- | | 1.65 (m, 2H), 1.42 (d, J = 6.5 Hz, 1H), 1.30-0.95 (m, 6H), 0.80-0.65 (m, 3H) ppm. |
| | b]pyridin-2-yl)acetonitrile | | MS: M/e 505 (M + 1)⁺. |
| A264 | 2-(7-((2S,5R)-5-ethyl-2-methyl-4- | 5 mg, 23% | ¹H NMR (400 MHz, CD₃OD) δ 8.95-8.80 (m, 2H), 8.14-8.00 (m, 3H), 5.49 (d, J = |
| | (1-(quinoxalin-6-yl)ethyl)piperazin- | | 7.5 Hz, 1H), 5.40 (d, J = 12.1 Hz, 2H), 4.09-3.91 (m, 1H), 3.65-3.46 (m, 1H), 3.40- |
| | 1-yl)-3-methyl-5-oxo-4,5-dihydro- | | 3.33 (m, 2H), 3.08-2.77 (m, 2H), 2.49 (d, J = 4.6 Hz, 3H), 2.45-2.22 (m, 1H), 1.63 |
| | 2H-pyrazolo[4,3-b]pyridin-2- | | (d, J = 7.5 Hz, 2H), 1.49-1.41 (m, 5H), 1.24 (d, J = 6.6 Hz, 2H), 1.13-0.69 (m, 3H). |
| | yl)acetonitrile | | MS: M/e 471 (M + 1)⁺ |
| A272 | 2-(7-((2S,5R)-4-(1-(2,6-dichloro-4- | 14 mg | ¹HNMR (400 MHz, CD₃OD) δ 7.92 (s, 1H), 7.36-7.30 (m, 1H), 7.24-7.15 (m, 1H), |
| | fluorophenyl)ethyl)-2,5- | | 5.56 (s, 1H), 5.48 (s, 2H), 4.79-4.23 (m, 2.5H), 3.64-3.50 (m, 1.5 H), 3.43 (s, 3H), |
| | dimethylpiperazin-1-yl)-4-methyl-5- | | 3.42-3.37 (m, 0.5H), 3.04-2.92 (m, 1H), 2.88-2.76 (m, 1H), 2.16-2.06 (m, 0.5H), |
| | oxo-4,5-dihydro-2H-pyrazolo[4,3- | | 1.56-1.42 (m, 3H), 1.38-1.31 (m, 1H), 1.19-1.04 (m, 5H) ppm. MS: M/e 491 |
| | b]pyridin-2-yl)acetonitrile | | (M + 1)⁺. |
| A273 | 2-(7-((2S,5R)-4-(1-(3,4-difluoro-2- | 6 mg | ¹H NMR (400 MHz, CD₃OD) δ 7.93 (s, 1H), 7.32 (s, 1H), 6.94 (d, J = 7.3 Hz, 1H), |
| | methoxyphenyl)ethyl)-2,5- | | 5.57 (s, 1H), 5.48 (s, 2H), 4.64 (s, 1H), 3.90 (s, 1H), 3.90 (d, J = 4.3 Hz, 3H), 3.61 (s, |
| | dimethylpiperazin-1-yl)-4-methyl-5- | | 2H), 3.44 (s, 3H), 2.82 (s, 2H), 2.27 (s, 1H), 1.36 (d, J = 6.5 Hz, 3H), 1.29-1.22 (m, |
| | oxo-4,5-dihydro-2H-pyrazolo[4,3- | | 3H), 1.11 (d, J = 48.1 Hz, 3H). MS: M/e 471 (M + 1)⁺. |
| | b]pyridin-2-yl)acetonitrile | | |
| A277 | 2-(7-((2S,5R)-4-(1-(4-chloro-2- | 12 mg, 37% | ¹H NMR (400 MHz, CD₃OD) δ 8.03 (dd, J = 8.5, 4.9 Hz, 1H), 7.93 (s, 1H), 7.73-7.58 |
| | (trifluoromethyl)phenyl)ethyl)-2,5- | | (m, 2H), 5.57 (s, 1H), 5.47 (s, 2H), 4.61 (s, 1H), 4.19-4.00 (m, 1H), 3.60-3.45 (m, |
| | diethylpiperazin-1-yl)-4-methyl-5- | | 1H), 3.43 (s, 3H), 3.28-3.18 (m, 1H), 3.18-3.00 (m, 1H), 2.91-2.61 (m, 1H), 2.36- |
| | oxo-4,5-dihydro-2H-pyrazolo[4,3- | | 2.07 (m, 2H), 1.95-1.45 (m, 3H), 1.36-1.26 (m, 3H), 1.03 (s, 3H), 0.78-0.54 (m, |
| | b]pyridin-2-yl)acetonitrile | | 3H). MS: M/e 535 (M + 1)⁺ |
| A283 | 2-(7-((2S,5R)-4-(1-(2- | 100 mg, 78% | ¹H NMR (400 MHz, DMSO-d6) δ 8.59 (d, J = 4.4 Hz, 1H), 7.99 (s, 1H), 7.96 (d, J = |
| | methoxyquinoxalin-6-yl)ethyl)-2,5- | | 7.3 Hz, 1H), 7.86 (s, 1H), 7.83 (s, 1H), 5.61 (s, 2H), 5.40 (d, J = 5.9 Hz, 1H), 5.00-4.30 |
| | dimethylpiperazin-1-yl)-4-methyl-5- | | (m, 1H), 4.04 (d, J = 2.5 Hz, 3H), 3.86 (q, J = 6.6 Hz, 0.5H), 3.72 (q, J = 6.4 Hz, 0.5H), |
| | oxo-4,5-dihydro-2H-pyrazolo[4,3- | | 3.62-3.44 (m, 1.5H), 3.28 (s, 3H), 3.25 (s, 1H), 2.95 (d, J = 8.0 Hz, 0.5H), 2.82 (d, J = |
| | b]pyridin-2-yl)acetonitrile | | 12.3 Hz, 1H), 2.71 (d, J = 8.2 Hz, 0.5H), 2.10 (d, J = 11.3 Hz, 0.5H), 1.35 (t, J = 5.7 |
| | | | Hz, 3H), 1.29 (d, J = 6.6 Hz, 1.5H), 1.10 (t, J = 5.7 Hz, 3H), 0.96 (d, J = 6.4 Hz, 1.5H) |
| | | | ppm. MS: M/e 487 (M + 1)⁺. |
| A285 | 2-(7-((2S,5R)-4-(1-(2- | 2.3 mg | ¹H NMR (400 MHz, DMSO-d6) δ 7.99 (s, 1H), 7.97-7.87 (m, 1H), 7.45-7.35 (m, 2H), |
| | (dimethylphosphoryl)-4- | | 5.62 (s, 2H), 5.45-5.36 (m, 1H), 4.52 (dd, J = 6.5 Hz, 1H), 3.60-3.40 (m, 2H), 3.28 (s, |
| | fluorophenyl)ethyl)-2,5- | | 3H), 3.26-3.18 (m, 1H), 3.03-2.89 (m, 1H), 2.82-2.76 (m, 1H), 1.99 (d, J = 12.3 Hz, |
| | dimethylpiperazin-1-yl)-4-methyl-5- | | 1H), 1.81-1.71 (m, 6H), 1.29-1.22 (m, 5H), 1.09-1.04 (m, 4H) ppm. MS: M/e 499 |
| | oxo-4,5-dihydro-2H-pyrazolo[4,3- | | (M + 1)⁺. |
| | b]pyridin-2-yl)acetonitrile | | |
| A288 | 2-(7-((2S,5R)-4-(1-(3,4-difluoro-2- | 2.7 mg | ¹H NMR (400 MHz, CD₃OD) δ 7.93-7.92 (m, 1H), 7.65-7.45 (m, 2H), 5.56 (s, 1H), |
| | (trifluoromethyl)phenyl)ethyl)-2,5- | | 5.47-5.46 (m, 2H), 4.29-4.27 (m, 0.5H), 4.11-4.09 (m, 0.5H), 3.52-3.49 (m, 1H), |
| | diethylpiperazin-1-yl)-4-methyl-5- | | 3.43 (s, 3H), 3.24-3.19 (m, 0.5H), 2.95-2.94 (m, 0.5H), 2.78-2.75 (m, 1H), 2.42- |
| | oxo-4,5-dihydro-2H-pyrazolo[4,3- | | 2.28 (m, 1H), 2.03-1.54 (m, 5H), 1.37-1.23 (m, 5H), 1.05-0.99 (m, 2H), 0.92- |
| | b]pyridin-2-yl)acetonitrile | | 0.90 (m, 1H), 0.81-0.78 (m, 1H), 0.69-0.65 (m, 1H). MS: M/e 537 (M + 1)⁺. |
| A289 | 2-(1-((2R,5S)-4-(2-(cyanomethyl)- | 8 mg, 37% | ¹H NMR (400 MHz, CD₃OD) δ 7.92 (s, 1H), 7.69-7.63 (m, 1H), 7.35-7.10 (m, 2H), |
| | 4-methyl-5-oxo-4,5-dihydro-2H- | | 5.56 (s, 1H), 5.47 (s, 2H), 4.72-4.38 (m, 1H), 4.23 (s, 1H), 3.43 (s, 3H), 3.05 (d, J = |
| | pyrazolo[4,3-b]pyridin-7-yl)-2,5- | | 11.8 Hz, 1H), 2.87 (d, J = 11.7 Hz, 1H), 2.46 (s, 1H), 2.08-1.99 (m, 1H), 1.95-1.67 |
| | diethylpiperazin-1-yl)ethyl)-5- | | (m, 2H), 1.50 (s, 1H), 1.41 (d, J = 5.8 Hz, 3H), 1.37-1.29 (m, 1H), 1.06-0.99 (m, |
| | fluorobenzamide | | 1H), 0.94 (s, 3H), 0.73 (s, 3H). MS: M/e 494 (M + 1)⁺ |
| A290 | methyl 2-(1-((2R,5S)-4-(2- | 5 mg | ¹H NMR (400 MHz, CD₃OD) δ 7.91 (s, 1H), 7.78 (dd, J = 8.7, 5.7 Hz, 1H), 7.43-7.36 |
| | (cyanomethyl)-4-methyl-5-oxo-4,5- | | (m, 1H), 7.29 (td, J = 8.6, 2.8 Hz, 1H), 5.55 (s, 1H), 5.46 (s, 2H), 4.52 (d, J = 6.2 Hz, |
| | dihydro-2H-pyrazolo[4,3-b]pyridin- | | 1H), 3.90 (s, 3H), 3.43 (s, 3H), 3.25 (d, J = 12.5 Hz, 1H), 2.96 (d, J = 11.9 Hz, 1H), |
| | 7-yl)-2,5-diethylpiperazin-1- | | 2.84 (d, J = 8.4 Hz, 1H), 2.35 (d, J = 9.0 Hz, 1H), 2.15-1.96 (m, 2H), 1.82-1.72 (m, |
| | yl)ethyl)-5-fluorobenzoate | | 1H), 1.34 (d, J = 6.5 Hz, 5H), 0.90 (dd, J = 8.6, 6.3 Hz, 4H), 0.71 (t, J = 7.4 Hz, 3H) |
| | | | ppm. MS: M/e 509 (M + 1)⁺ |
| A291 | 2-(1-((2R,5S)-4-(2-(cyanomethyl)- | 7 mg | ¹H NMR (400 MHz, CD₃OD) δ 7.92 (s, 1H), 7.83-7.46 (m, 2H), 7.24-7.06 (m, 2H), |
| | 4-methyl-5-oxo-4,5-dihydro-2H- | | 5.55 (s, 1H), 5.46 (s, 2H), 4.14 (d, J = 6.5 Hz, 1H), 3.88-3.69 (m, 1H), 3.43 (s, 3H), |
| | pyrazolo[4,3-b]pyridin-7-yl)-2,5- | | 3.01 (s, 1H), 2.90 (d, J = 4.6 Hz, 3H), 2.45-2.32 (m, 1H), 2.11-2.02 (m, 1H), 1.85- |
| | diethylpiperazin-1-yl)ethyl)-5- | | 1.78 (m, 1H), 1.68 (d, J = 6.8 Hz, 1H), 1.54-1.47 (m, 1H), 1.35 (dd, J = 13.9, 6.4 Hz, |
| | fluoro-N-methylbenzamide | | 4H), 1.07-0.82 (m, 5H), 0.73 (t, J = 7.2 Hz, 3H) ppm. MS: M/e 508 (M + 1)+ |
| A292 | 2-(1-((2R,5S)-4-(2-(cyanomethyl)- | 8 mg | ¹H NMR (400 MHz, CD₃OD) δ 7.92 (s, 1H), 7.77 (s, 1H), 7.19 (d, J = 8.4 Hz, 1H), |
| | 4-methyl-5-oxo-4,5-dihydro-2H- | | 6.97 (s, 1H), 5.56 (s, 1H), 5.46 (s, 2H), 3.83 (d, J = 5.7 Hz, 1H), 3.43 (s, 3H), 3.12 (s, |
| | pyrazolo[4,3-b]pyridin-7-yl)-2,5- | | 3H), 3.00 (s, 1H), 2.97-2.83 (m, 4H), 2.63-2.39 (m, 1H), 2.15-2.02 (m, 1H), 1.87- |
| | diethylpiperazin-1-yl)ethyl)-5- | | 1.78 (m, 1H), 1.77-1.39 (m, 3H), 1.31 (s, 4H), 1.08-0.85 (m, 4H), 0.77 (t, J = 6.8 |
| | fluoro-N,N-dimethylbenzamide | | Hz, 3H) ppm. MS: M/e 522 (M + 1)⁺ |
| A293 | 2-(7-((2S,5R)-2,5-diethyl-4-(1-(4- | 1 mg | ¹H NMR (400 MHz, CD₃OD) δ 7.92 (s, 2H), 7.43 (d, J = 7.5 Hz, 2H), 5.55 (s, 1H), |
| | fluoro-2- | | 5.47 (s, 2H), 4.01 (m, 1H), 3.50-3.40 (m, 4H), 3.26-2.94 (m, 2H), 2.69 (s, 1H), 2.30 |
| | (trifluoromethyl)phenyl)propyl)pipe | | (m, 1H), 2.14 (d, J = 12.1 Hz, 1H), 2.02 (m, 1H), 1.87 (s, 1H), 1.69 (m, 2H), 1.59 (m, |
| | razin-1-yl)-4-methyl-5-oxo-4,5- | | 1H), 1.49 (m, 1H), 1.03 (s, 3H), 0.89 (d, J = 6.9 Hz, 1H), 0.75 (t, J = 7.3 Hz, 2H), 0.62 |
| | dihydro-2H-pyrazolo[4,3-b]pyridin- | | (m, 3H). MS: M/e 533 (M + 1)⁺. |
| | 2-yl)acetonitrile | | |
| A299 | 2-allyl-7-((2S,5R)-2,5-dimethyl-4- | 15 mg, 27% | (21 mg, 37%). ¹H NMR (400 MHz, CD₃OD) δ 8.88-8.87 (m, 2H), 8.08-8.03 (m, |
| | ((S)-1-(quinoxalin-6- | | 3H), 7.79 (s, 1H), 6.08-6.03 (m, 1H), 5.53 (s, 1H), 5.23-5.18 (m, 2H), 4.95-4.88 |
| | yl)ethyl)piperazin-1-yl)-4-methyl- | | (m, 3H), 4.32 (br s, 1H), 3.97-3.92 (m, 1H), 3.45-3.41 (m, 4H), 3.09-3.07 (m, 1H), |
| | 2,4-dihydro-5H-pyrazolo[4,3- | | 2.93-2.90 (m, 2H), 1.48-1.44 (m, 6H), 1.09-1.03 (m, 3H) ppm. MS: M/e 458 |
| | b]pyridin-5-one, or 2-allyl-7- | | (M + 1)⁺. |
| | ((2S,5R)-2,5-dimethyl-4-((R)-1- | | |
| | (quinoxalin-6-yl)ethyl)piperazin-1- | | |
| | yl)-4-methyl-2,4-dihydro-5H- | | |
| | pyrazolo[4,3-b]pyridin-5-one | | |
| A300 | 2-(but-3-en-1-yl)-7-((2S,5R)-2,5- | 15 mg, 27% | ¹H NMR (400 MHz, CD₃OD) δ 8.88-8.87 (m, 2H), 8.11-8.03 (m, 3H), 7.79 (s, 1H), |
| | dimethyl-4-((S)-1-(quinoxalin-6- | | 5.81-5.77 (m, 1H), 5.52 (s, 1H), 5.05-4.98 (m, 3H), 4.33-4.29 (m, 3H), 3.99-3.95 |
| | yl)ethyl)piperazin-1-yl)-4-methyl- | | (m, 1H), 3.46-3.43 (m, 4H), 3.09-3.07 (m, 1H), 2.93-2.90 (m, 2H), 2.67-2.62 (m, |
| | 2,4-dihydro-5H-pyrazolo[4,3- | | 2H), 1.48-1.44 (m, 6H), 1.09-1.03 (m, 3H) ppm. MS: M/e 472 (M + 1)⁺. |
| | b]pyridin-5-one, or 2-(but-3-en-1- | | |
| | yl)-7-((2S,5R)-2,5-dimethyl-4-((R)- | | |
| | 1-(quinoxalin-6-yl)ethyl)piperazin- | | |
| | 1-yl)-4-methyl-2,4-dihydro-5H- | | |
| | pyrazolo[4,3-b]pyridin-5-one | | |
| A305 | 2-(7-((2S,5R)-4-(1-(isoquinolin-3- | 56 mg | ¹H NMR (400 MHz, DMSO-d₆) δ 9.21 (d, J = 9.4 Hz, 1H), 8.09 (d, J = 8.6 Hz, 1H), |
| | yl)ethyl)-2,5-dimethylpiperazin-1- | | 7.99-7.86 (m, 3H), 7.77 (s, 1H), 7.65 (s, 1H), 5.57 (s, 1H), 5.47 (d, J = 3.3 Hz, 2H), |
| | yl)-4-methyl-5-oxo-4,5-dihydro-2H- | | 4.68-4.25 (m, 1H), 4.10-3.78 (m, 1H), 3.76-3.62 (m, 1H), 3.51 (d, J = 14.2 Hz, |
| | pyrazolo[4,3-b]pyridin-2- | | 1H), 3.43 (s, 3H), 3.16-2.18 (m, 3H), 1.49-1.44 (m, 3H), 1.43-1.25 (m, 3H), 1.22- |
| | yl)acetonitrile | | 1.03 (m, 3H) ppm. MS: M/e 456 (M + 1)⁺. |
| A306 | 2-(7-((2S,5R)-2,5-dimethyl-4-(1-(2- | 32 mg, 41% a mixture of | ¹H NMR (400 MHz, CD₃OD) δ 8.23 (dd, J = 8.1, 5.6 Hz, 1H), 8.01-7.80 (m, 3H), |
| | methylquinolin-7- | diastereomers | 7.70 (d, J = 8.6 Hz, 1H), 7.41 (dd, J = 8.2, 5.2 Hz, 1H), 5.56 (s, 1H), 5.46 (d, J = 3.6 |
| | yl)ethyl)piperazin-1-yl)-4-methyl-5- | | Hz, 2H), 4.97-4.89 (m, 1H), 4.57 (s, 1H), 3.93-3.75 (m, 1H), 3.71-3.65 (m, 1H), |
| | oxo-4,5-dihydro-2H-pyrazolo[4,3- | | 3.62-3.45 (m, 1H), 3.43 (s, 3H), 3.11-2.80 (m, 2H), 2.72 (s, 3H), 1.57-1.43 (m, |
| | b]pyridin-2-yl)acetonitrile | | 3H), 1.44-1.22 (m, 3H), 1.22-1.00 (m, 3H). MS: M/e 470 (M + 1)⁺. |
| A307 | 7-(1-((2R,5S)-4-(2-(cyanomethyl)- | 10 mg, 9% as a mixture of | ¹H NMR (400 MHz, CD₃OD) δ 8.35 (d, J = 11.0 Hz, 1H), 8.04-7.91 (m, 4H), 7.85 |
| | 4-methyl-5-oxo-4,5-dihydro-2H- | diastereomers | (d, J = 8.5 Hz, 1H), 7.68-7.56 (m, 1H), 5.56 (s, 1H), 5.46 (d, J = 3.6 Hz, 2H), 4.61 (s, |
| | pyrazolo[4,3-b]pyridin-7-yl)-2,5- | | 1H), 4.25-3.85 (m, 1H), 3.75 (d, J = 6.6 Hz, 1H), 3.68 (d, J = 9.8 Hz, 1H), 3.43 (s, |
| | dimethylpiperazin-1-yl)ethyl)-2- | | 3H), 3.06 (d, J = 8.4 Hz, 1H), 2.91-2.84 (m, 1H), 2.20 (d, J = 10.8 Hz, 1H), 1.48- |
| | naphthonitrile | | 1.39 (m, 5H), 1.20 (t, J = 7.3 Hz, 3H), 1.04 (d, J = 6.4 Hz, 1H) ppm. MS: M/e |
| | | | 480(M + 1)⁺. |
| A308 | 2-(7-((2S,5R)-2,5-dimethyl-4-((S)- | 70 mg, 43% as a single | ¹H NMR (400 MHz, CD₃OD) δ 8.79 (s, 1H), 8.06 (d, J = 8.5 Hz, 1H), 7.99 (s, 1H), |
| | 1-(3-methylquinoxalin-6- | diastereoisomer | 7.94 (d, J = 8.3 Hz, 1H), 7.74 (s, 1H), 5.49 (s, 1H), 5.43 (s, 2H), 5.09-4.86 (m, 1H), |
| | yl)ethyl)piperazin-1-yl)-5-oxo-4,5- | | 4.56-4.16 (m, 1H), 3.95 (d, J = 6.5 Hz, 1H), 3.48 (d, J = 9.7 Hz, 1H), 3.11-3.04 (m, |
| | dihydro-2H-pyrazolo[4,3-b]pyridin- | | 1H), 2.96-2.87 (m, 2H), 2.77 (s, 3H), 1.46 (d, J = 6.5 Hz, 3H), 1.43 (d, J = 6.6 Hz, |
| | 2-yl)acetonitrile | | 3H), 1.06 (d, J = 6.4 Hz, 3H). MS: M/e 457 (M + 1)⁺. |
| A320 | 2-(7-((2S,5R)-2,5-dimethyl-4-(1-(2- | 1 mg, 6% as a mixture of | ¹H NMR (400 MHz, CDCl₃) δ7.94 (d, J = 19.0 Hz, 1H), 7.82-7.73 (m, 1H), 7.41 (dd, |
| | methylbenzo[d]thiazol-5- | diastereomers | J = 18.6, 11.5 Hz, 2H), 6.96 (s, 1H), 5.61 (d, J = 5.1 Hz, 1H), 5.12 (d, J = 3.8 Hz, 2H), |
| | yl)ethyl)piperazin-1-yl)-4-methyl-5- | | 3.76-3.56 (m, 2H), 3.38 (d, J = 24.7 Hz, 4H), 3.03-2.71 (m, 5H), 2.55-2.29 (m, |
| | oxo-4,5-dihydro-2H-pyrazolo[4,3- | | 1H), 2.06 (s, 1H), 1.57 (s, 3H), 1.18-1.12 (m, 3H), 0.87 (d, J = 7.1 Hz, 3H) ppm. MS: |
| | b]pyridin-2-yl)acetonitrile | | M/e 476 (M + 1)⁺. |
| A321 | 2-(7-((2S,5R)-2,5-dimethyl-4-(1-(2- | 13 mg, 22% as a mixture of | ¹H NMR (400 MHz, CD₃OD) δ 8.66 (d, J = 13.8 Hz, 1H), 7.93 (s, 1H), 7.88-7.77 (m, |
| | methyl-[1,2,4]triazolo[1,5- | diastereomers | 1H), 7.66 (t, J = 10.0 Hz, 1H), 5.57 (s, 1H), 5.47 (d, J = 2.0 Hz, 2H), 4.64 (s, 1H), 3.92- |
| | alpyridin-6-yl)ethyl)piperazin-1-yl)- | | 3.46 (m, 3H), 3.44 (s, 3H), 3.13-2.92 (m, 1H), 2.86 (d, J = 11.4 Hz, 1H), 2.54 (d, J = |
| | 4-methyl-5-oxo-4,5-dihydro-2H- | | 2.7 Hz, 3H), 2.27 (d, J = 11.9 Hz, 1H), 1.44-1.40 (m, 3H), 1.32-1.20 (m, 3H), 1.20- |
| | pyrazolo[4,3-b]pyridin-2- | | 1.04(m, 3H) ppm. MS: M/e 460 (M + 1)⁺. |
| | yl)acetonitrile | | |
| A327 | 2-(7-((2S,5R)-2,5-diethyl-4-(1-(4- | 19 mg 25% as a mixture of | Compound A327: ¹H NMR (400 MHz, CD₃OD) δ 7.92 (s, 1H), 7.73-7.59 (m, 1H), |
| | fluoro-2-(1- | diastereomers | 7.15-7.06 (m, 1H), 7.05-6.97 (m, 1H), 5.57 (s, 1H), 5.46 (s, 2H), 4.06-3.96 (m, |
| | methoxyethyl)phenyl)ethyl)piperazin- | | 1H), 3.43 (s, 3H), 3.24 (d, J = 11.4 Hz, 3H), 3.19-3.07 (m, 1H), 2.91-2.70 (m, 2H), |
| | 1-yl)-4-methyl-5-oxo-4,5- | | 2.48-2.32 (m, 1H), 2.22-2.07 (m, 1H), 1.67-1.54 (m, 2H), 1.38-1.27 (m, 10H), |
| | dihydro-2H-pyrazolo[4,3-b]pyridin- | | 1.08-0.92 (m, 3H), 0.74-0.55 (m, 3H). MS: M/e 509 (M + 1)⁺. |
| | 2-yl)acetonitrile | | |
| A330 | 2-(7-((2S,5R)-2,5-dimethyl-4-(1-(3- | 21 mg as a mixture of | ¹H NMR (400 MHz, DMSO-d₆) δ 7.92 (s, 1H), 7.73 (d, J = 20.5 Hz, 1H), 7.70-7.62 |
| | (trifluoromethyl)phenyl)ethyl)piperazin- | diastereomers | (m, 1H), 7.57-7.44 (m, 2H), 5.57 (s, 1H), 5.47 (s, 2H), 4.75-4.12 (m, 2H), 3.82- |
| | 1-yl)-4-methyl-5-oxo-4,5- | | 3.56 (m, 2H), 3.43 (s, 3H), 3.40-3.35 (m, 1H), 3.03-2.06 (m, 2H), 1.48-1.36 (m, |
| | dihydro-2H-pyrazolo[4,3-b]pyridin- | | 3H), 1.35-1.18 (m, 3H), 1.16-0.98 (m, 3H) ppm. MS: M/e 473 (M + 1)⁺ |
| | 2-yl)acetonitrile | | |
| A333 | 2-(7-((2S,5R)-4-(di-p-tolylmethyl)- | 5 mg, 5% | ¹H NMR (400 MHz, CD₃OD) δ 7.91 (s, 1H), 7.39 (d, J = 7.8 Hz, 4H), 7.09 (t, J = 8.6 |
| | 2,5-dimethylpiperazin-1-yl)-4- | | Hz, 4H), 5.55 (s, 1H), 5.45 (s, 2H), 4.47 (s, 1H), 3.59 (d, J = 9.3 Hz, 1H), 3.43 (s, 3H), |
| | methyl-5-oxo-4,5-dihydro-2H- | | 3.32 (s, 1H), 3.20 (s, 1H), 2.83 (d, J = 10.6 Hz, 2H), 2.48 (d, J = 12.1 Hz, 1H), 2.27 (d, |
| | pyrazolo[4,3-b]pyridin-2- | | J = 4.8 Hz, 6H), 1.39 (d, J = 6.6 Hz, 3H), 1.01 (d, J = 6.4 Hz, 3H). MS: M/e 495 (M + 1)⁺. |
| | yl)acetonitrile | | |
| A334 | 2-(but-2-yn-1-yl)-7-((2S,5R)-2,5- | A334a | A334a (the earlier peak): ¹H NMR (400 MHz, CD₃OD) δ 7.90 (s, 1H), 7.81 (t, J = 7.2 |
| | diethyl-4-(1-(2-fluoro-4- | (15 mg) | Hz, 1H), 7.55 (d, J = 8.0 Hz, 1H), 7.46 (d, J = 10.0 Hz, 1H), 5.53 (s, 1H), 5.03 (s, 2H), |
| | (trifluoromethyl)phenyl)ethyl)piperazin- | | 4.42-4.30 (m, 1H), 3.44 (s, 3H), 3.38-3.32 (m, 1H), 3.08-2.95 (m, 2H), 2.66 (s, 1H), |
| | 1-yl)-4-methyl-2,4-dihydro-5H- | | 2.53-2.42 (m, 1H), 2.12-1.97 (m, 1H), 1.91-1.75 (m, 4H), 1.66-1.49 (m, 3H), 1.43 |
| | pyrazolo[4,3-b]pyridin-5-one | | (d, J = 6.4 Hz, 3H), 0.95 (t, J = 7.2 Hz, 3H), 0.78 (t, J = 7.2 Hz, 3H) ppm. MS: M/e 532 |
| | | | (M + 1)⁺. |
| | | A334b | A334b (the later peak): ¹H NMR (400 MHz, CD₃OD) δ 7.93-7.82 (m, 2H), 7.50 (d, J = |
| | | (15 mg) | 8.0 Hz, 1H), 7.41 (d, J = 10.0 Hz, 1H), 5.52 (s, 1H), 5.04 (s, 2H), 4.13-4.04 (m, |
| | | | 1H), 3.54-3.45 (m, 1H), 3.44 (s, 3H), 3.30-3.31 (m, 1H), 3.17-3.08 (m, 1H), 2.76- |
| | | | 2.67 (m, 1H), 2.29 (d, J = 12.0 Hz, 1H), 2.01-1.89 (m, 1H), 1.85 (s, 3H), 1.76-1.46 |
| | | | (m, 4H), 1.34 (d, J = 6.4 Hz, 3H), 1.02 (t, J = 7.2 Hz, 3H), 0.65 (t, J = 7.2 Hz, 3H) ppm. |
| | | | MS: M/e 532 (M + 1)⁺. |
| A336 | 2-(but-2-yn-1-yl)-7-((2S,5R)-2,5- | 65 mg, 54% as a single | ¹H NMR (400 MHz, CD₃OD)δ 8.78 (s, 1H), 8.09-7.86 (m, 4H), 5.53 (s, 1H), 5.03 (d, |
| | dimethyl-4-((S)-1-(3- | diastereoisomer | J = 2.0 Hz, 2H), 4.98-4.89 (m, 1H), 4.39-4.17 (m, 1H), 3.94 (q, J = 6.4 Hz, 1H), |
| | methylquinoxalin-6- | | 3.44 (s, 3H), 3.41 (d, J = 2.8 Hz, 1H), 3.10-3.02 (m, 1H), 2.95-2.85 (m, 2H), 2.76 |
| | yl)ethyl)piperazin-1-yl)-4-methyl- | | (s, 3H), 1.85 (s, 3H), 1.42 (d, J = 5.2 Hz, 6H), 1.04 (d, J = 6.4 Hz, 3H) ppm. MS: M/e |
| | 2,4-dihydro-5H-pyrazolo[4,3- | | 484 (M + 1)⁺ |
| | b]pyridin-5-one | | |
| A338 | 2-(4-(1-((2R,5S)-4-(2- | 7.5 mg, 10% | ¹H NMR (400 MHz, CD₃OD) δ7.92 (s, 1H), 7.55-7.40 (m, 4H), 5.55 (s, 1H), 5.47 (s, |
| | (cyanomethyl)-4-methyl-5-oxo-4,5- | | 2H), 3.86-3.40 (m, 1H), 3.56-3.39 (m, 4H), 3.16 (d, J = 9.0 Hz, 1H), 3.08-2.85 (m, |
| | dihydro-2H-pyrazolo[4,3-b]pyridin- | | 1H), 2.70-2.32 (m, 2H), 2.17-1.92 (m, 1H), 1.87-1.63 (m, 8H), 1.54 (d, J = 6.8 Hz, |
| | 7-yl)-2,5-diethylpiperazin-1- | | 2H), 1.32 (dd, J = 15.8, 9.5 Hz, 3H), 1.06-0.95 (m, 3H), 0.74-0.58 (m, 3H) ppm. MS: |
| | yl)ethyl)phenyl)-2- | | M/e 500 (M + 1)⁺. |
| | methylpropanenitrile | | |
| A340 | 2-(7-((2S,5R)-2,5-diethyl-4-(1-(2- | 2 mg, 4% as a mixture of | ¹H NMR (400 MHz, CD₃OD) δ 7.92 (s, 1H), 7.50-7.35 (m, 1H), 7.04-6.95 (m, 1H), |
| | fluoro-4- | diastereomers | 6.88 (t, J = 11.4 Hz, 1H), 5.54 (s, 1H), 5.47 (s, 2H), 4.17-3.95 (m, 1H), 3.46 (d, J = |
| | methylphenyl)ethyl)piperazin-1-yl)- | | 24.2 Hz, 4H), 3.30-3.20 (m, 2H), 2.93 (s, 1H), 2.70-2.35 (m, 2H), 2.33 (d, J = 5.0 Hz, |
| | 4-methyl-5-oxo-4,5-dihydro-2H- | | 3H), 2.10-1.90 (m, 1H), 1.86-1.42 (m, 3H), 1.32 (dd, J = 18.8, 6.2 Hz, 3H), 1.06-0.92 |
| | pyrazolo[4,3-b]pyridin-2- | | (m, 3H), 0.77-0.62 (m, 3H) ppm. MS: M/e 465 (M + 1)⁺ |
| | yl)acetonitrile | | |
| A342 | 2-(7-((2S,5R)-4-(1-(4-((2,2- | 2 mg, 2% as a mixture of | ¹H NMR (400 MHz, CD₃OD) δ 7.92 (s, 1H), 7.45-7.29 (m, 4H), 5.55 (s, 1H), 5.47 (s, |
| | dimethylmorpholino)methyl)phenyl) | diastereomers | 2H), 3.87-3.48 (m, 6H), 3.43 (s, 3H), 3.19 (s, 1H), 3.08-2.91 (m, 1H), 2.72-2.47 |
| | ethyl)-2,5-diethylpiperazin-1-yl)-4- | | (m, 3H), 2.45-2.22 (m, 3H), 2.12-1.78 (m, 2H), 1.73-1.48 (m, 3H), 1.37 (dd, J = |
| | methyl-5-oxo-4,5-dihydro-2H- | | 20.6, 5.8 Hz, 3H), 1.23 (d, J = 5.7 Hz, 6H), 1.08-0.93 (m, 3H), 0.72-0.51 (m, 3H) |
| | pyrazolo[4,3-b]pyridin-2- | | ppm. MS: M/e 560 (M + 1)⁺ |
| | yl)acetonitrile | | |
| A344 | 2-(7-((2S,5R)-4-(1-(4-((4,4- | 6 mg as a mixture of | ¹H NMR (400 MHz, CD3OD) δ 7.92 (s, 1H), 7.41-7.28 (m, 4H), 5.54 (d, J = 4.0 Hz, |
| | difluoropiperidin-1- | diastereomers | 1H), 5.46 (d, J = 3.2 Hz, 2H), 3.81-3.71 (m, 1H), 3.61 (s, 2H), 3.53-3.44 (m, 1H), |
| | yl)methyl)phenyl)ethyl)-2,5- | | 3.43 (s, 3H), 3.27-2.83 (m, 2H), 2.67-2.55 (m, 5H), 2.48-2.34 (m, 1H), 2.16-1.91 |
| | diethylpiperazin-1-yl)-4-methyl-5- | | (m, 6H), 1.86-1.47 (m, 4H), 1.39-1.30 (m, 3H), 0.99 (dt, J = 25.6, 7.2 Hz, 3H), 0.63 |
| | oxo-4,5-dihydro-2H-pyrazolo[4,3- | | (dt, J = 32.8, 7.2 Hz, 3H) ppm. MS: M/e 566 (M + 1)⁺ |
| | b]pyridin-2-yl)acetonitrile | | |
| A346 | 2-(7-((2S,5R)-2,5-diethyl-4-(1-(5- | 12 mg as a mixture of | ¹H NMR (400 MHz, CD₃OD) δ 8.35 (s, 1H), 7.94 (d, J = 5.2 Hz, 1H), 7.70 (d, J = 8.0 |
| | methylpyridin-2-yl)ethyl)piperazin- | diastereomers | Hz, 1H), 7.51 (t, J = 8.0 Hz, 1H), 5.57 (d, J = 4.8 Hz, 1H), 5.48 (d, J = 4.0 Hz, 2H), |
| | 1-yl)-4-methyl-5-oxo-4,5-dihydro- | | 4.07-3.90 (m, 1H), 3.62-3.53 (m, 0.5H), 3.44 (s, 3H), 3.41-3.22 (m, 3H), 3.12-2.87 |
| | 2H-pyrazolo[4,3-b]pyridin-2- | | (m, 1.5H), 2.56-2.41 (m, 1H), 2.37 (s, 3H), 2.14-1.53 (m, 4H), 1.41 (s, 3H), 1.10- |
| | yl)acetonitrile | | 0.91 (m, 3H), 0.78-0.64 (m, 3H) ppm. MS: M/e 448 (M + 1)⁺. |
| A349 | 2-(7-((2S,5R)-2,5-diethyl-4-(1-(6- | A349a | A349a (the earlier peak): ¹H NMR (400 MHz, CD₃OD) δ 8.37 (s, 1H), 7.92 (s, 1H), |
| | methylpyridin-3-yl)ethyl)piperazin- | (17 mg) | 7.78 (d, J = 7.7 Hz, 1H), 7.31 (d, J = 7.9 Hz, 1H), 5.56 (s, 1H), 5.46 (s, 2H), 3.82 (d, J = |
| | 1-yl)-4-methyl-5-oxo-4,5-dihydro- | | 6.1 Hz, 1H), 3.43 (s, 3H), 3.27 (s, 3H), 3.00 (d, J = 11.7 Hz, 1H), 2.89 (d, J = 11.6 |
| | 2H-pyrazolo[4,3-b]pyridin-2- | | Hz, 1H), 2.53 (s, 3H), 2.38 (s, 1H), 2.16-2.02 (m, 1H), 1.88-1.75 (m, 1H), 1.59- |
| | yl)acetonitrile | | 1.49 (m, 2H), 1.37 (d, J = 6.2 Hz, 3H), 0.95 (t, J = 7.0 Hz, 3H), 0.72 (t, J = 7.3 Hz, 3H). |
| | | | MS: M/e 448 (M + 1)⁺ |
| | | A349b(29 | A349b (the later peak): ¹H NMR (400 MHz, CD₃OD) δ 8.41 (s, 1H), 7.92 (s, 1H), 7.79 |
| | | mg) | (d, J = 8.0 Hz, 1H), 7.28 (d, J = 7.6 Hz, 1H), 5.55 (s, 1H), 5.47 (s, 2H), 3.67 (d, J = 5.5 |
| | | | Hz, 1H), 3.50 (d, J = 13.9 Hz, 1H), 3.43 (s, 3H), 3.29-3.25 (m, 2H), 3.15 (d, J = 10.8 |
| | | | Hz, 1H), 2.68 (d, J = 10.8 Hz, 1H), 2.52 (s, 3H), 2.32 (d, J = 12.9 Hz, 1H), 1.97-1.86 |
| | | | (m, 1H), 1.72-1.61 (m, 2H), 1.59-1.50 (m, 1H), 1.33 (d, J = 6.0 Hz, 3H), 1.02 (t, J = |
| | | | 6.9 Hz, 3H), 0.63 (t, J = 7.3 Hz, 3H). MS: M/e 448 (M + 1)⁺ |
| A353 | 2-(7-((2S,5R)-2,5-diethyl-4-(1-(6- | A353a | A353a (the earlier peak): ¹H NMR (400 MHz, CD₃OD) δ 8.19 (s, 1H), 8.03 (t, J = 7.4 |
| | fluoropyridin-3-yl)ethyl)piperazin- | (22 mg) | Hz, 1H), 7.93 (s, 1H), 7.06 (d, J = 8.6 Hz, 1H), 5.56 (s, 1H), 5.47 (s, 2H), 4.85-3.90 |
| | 1-yl)-4-methyl-5-oxo-4,5-dihydro- | | (m, 2H), 3.72 (d, J = 5.4 Hz, 1H), 3.50 (d, J = 13.9 Hz, 1H), 3.43 (s, 3H), 3.15 (d, J = |
| | 2H-pyrazolo[4,3-b]pyridin-2- | | 10.1 Hz, 1H), 2.70 (d, J = 13.1 Hz, 1H), 2.31 (d, J = 12.7 Hz, 1H), 1.95-1.85 (m, 1H), |
| | yl)acetonitrile | | 1.74-1.62 (m, 2H), 1.59-1.51 (m, 1H), 1.34 (d, J = 6.3 Hz, 3H), 1.02 (t, J = 7.0 Hz, |
| | | | 3H), 0.64 (t, J = 7.2 Hz, 3H). MS: M/e 452 (M + 1)⁺. |
| | | A353b(19 | A353b (the later peak): ¹H NMR (400 MHz, CD₃OD) δ 8.16 (s, 1H), 8.02 (t, J = 7.9 Hz, |
| | | mg) | 1H), 7.92 (s, 1H), 7.09 (d, J = 8.7 Hz, 1H), 5.56 (s, 1H), 5.46 (s, 2H), 4.86-4.16 (m, |
| | | | 2H), 3.88 (d, J = 6.8 Hz, 1H), 3.43 (s, 3H), 3.30-3.25 (m, 1H), 3.00 (d, J = 12.0 Hz, |
| | | | 1H), 2.89 (d, J = 11.4 Hz, 1H), 2.37 (d, J = 6.1 Hz, 1H), 2.15-2.03 (m, 1H), 1.88- |
| | | | 1.77 (m, 1H), 1.55 (d, J = 7.4 Hz, 2H), 1.37 (d, J = 6.2 Hz, 3H), 0.95 (t, J = 6.9 Hz, |
| | | | 3H), 0.74 (t, J = 7.0 Hz, 3H). MS: M/e 452 (M + 1)⁺. |
| A355 | 2-(7-((2S,5R)-4-(1-(5,6- | A355a | A355a (the earlier peak): ¹H NMR (400 MHz, CD₃OD) δ 8.48 (s, 1H), 7.93 (s, 1H), |
| | dimethylpyrazin-2-yl)ethyl)-2,5- | (9 mg) | 5.56 (s, 1H), 5.47 (s, 2H), 4.87-3.85 (m, 2H), 3.84-3.71 (m, 1H), 3.54 (d, J = 13.2 |
| | diethylpiperazin-1-yl)-4-methyl-5- | | Hz, 1H), 3.43 (s, 3H), 3.15 (d, J = 10.4 Hz, 1H), 2.79 (d, J = 12.8 Hz, 1H), 2.55 (s, 3H), |
| | oxo-4,5-dihydro-2H-pyrazolo[4,3- | | 2.53 (s, 3H), 2.30 (d, J = 12.0 Hz, 1H), 2.03-1.86 (m, 1H), 1.84-1.60 (m, 2H), 1.59- |
| | b]pyridin-2-yl)acetonitrile | | 1.45 (m, 1H), 1.37 (d, J = 6.0 Hz, 3H), 1.02 (t, J = 7.2 Hz, 3H), 0.68 (t, J = 7.2 Hz, |
| | | | 3H). MS: M/e 463 (M + 1)⁺. |
| | | A355b(9 | A355b (the later peak): ¹H NMR (400 MHz, CD₃OD) δ 8.41 (s, 1H), 7.92 (s, 1H), 5.56 |
| | | mg) | (s, 1H), 5.47 (s, 2H), 4.85-4.10 (m, 2H), 4.05-3.92 (m, 1H), 3.43 (s, 3H), 3.39-3.32 |
| | | | (m, 1H), 2.98 (s, 2H), 2.56 (s, 3H), 2.54 (s, 3H), 2.43 (s, 1H), 2.11-1.95 (m, 1H), 1.90- |
| | | | 1.70 (m, 1H), 1.65-1.48 (m, 2H), 1.42 (d, J = 6.4 Hz, 3H), 0.94 (t, J = 7.2 Hz, 3H), |
| | | | 0.76 (t, J = 7.2 Hz, 3H). MS: M/e 463 (M + 1)⁺. |
| A356 | 2-(7-((2S,5R)-2,5-diethyl-4-(1-(5- | 45 mg, 33% as a mixture of | ¹H NMR (400 MHz, CD₃OD) δ 8.72-8.58 (m, 1H), 8.51-8.42 (m, 1H), 7.96-7.88 |
| | methylpyrazin-2-yl)ethyl)piperazin- | diastereomers | (m, 1H), 5.56 (s, 1H), 5.47 (d, J = 3.6 Hz, 2H), 4.71-4.15 (m, 1H), 4.09-3.78 (m, |
| | 1-yl)-4-methyl-5-oxo-4,5-dihydro- | | 1H), 3.59-3.31 (m, 5H), 3.19-2.75 (m, 2H), 2.60-2.52 (m, 3H), 2.49-2.23 (m, |
| | 2H-pyrazolo[4,3-b]pyridin-2- | | 1H), 2.06-1.48 (m, 4H), 1.46-1.35 (m, 3H), 1.10-0.86 (m, 3H), 0.82-0.62 (m, |
| | yl)acetonitrile | | 3H). MS: M/e 449 (M + 1)⁺. |
| A357 | 2-(7-((2S,5R)-2,5-diethyl-4-(1- | 50 mg as a mixture of | A357a (the earlier peak): ¹H NMR (400 MHz, CD₃OD) δ 8.53 (s, 1H), 7.93 (s, 1H), |
| | (5,6,7,8-tetrahydroquinoxalin-2- | diastereomers, | 5.56 (s, 1H), 5.47 (s, 2H), 4.86-3.86 (m, 2H), 3.77 (q, J = 6.4 Hz, 1H), 3.54 (d, J = |
| | yl)ethyl)piperazin-1-yl)-4-methyl-5- | A357a | 13.6 Hz, 1H), 3.43 (s, 3H), 3.15 (d, J = 10.8 Hz, 1H), 3.04-2.85 (m, 4H), 2.79 (dd, J = |
| | oxo-4,5-dihydro-2H-pyrazolo[4,3- | (15 mg) | 12.0, 3.2 Hz, 1H), 2.29 (d, J = 12.0 Hz, 1H), 2.03-1.87 (m, 5H), 1.80-1.61 (m, |
| | b]pyridin-2-yl)acetonitrile | | 2H), 1.58-1.44 (m, 1H), 1.37 (d, J = 6.8 Hz, 3H), 1.02 (t, J = 7.2 Hz, 3H), 0.68 (t, J = |
| | | | 7.2 Hz, 3H). MS: M/e 489 (M + 1)⁺. |
| | | A357b | A357b (the later peak): ¹H NMR (400 MHz, CD₃OD) δ 8.46 (s, 1H), 7.92 (s, 1H), 5.56 |
| | | (15 mg) | (s, 1H), 5.47 (s, 2H), 4.86-4.07 (m, 2H), 3.97 (q, J = 6.4 Hz, 1H), 3.43 (s, 3H), 3.39- |
| | | | 3.32 (m, 1H), 2.96 (dd, J = 9.6, 3.2 Hz, 6H), 2.50-2.35 (m, 1H), 2.15-1.87 (m, |
| | | | 5H), 1.88-1.72 (m, 1H), 1.66-1.51 (m, 2H), 1.42 (d, J = 6.8, 3H), 0.94 (t, J = 7.6 Hz, |
| | | | 3H), 0.76 (t, J = 7.2 Hz, 3H). MS: M/e 489 (M + 1)⁺. |
| A358 | 2-(7-((2S,5R)-2,5-diethyl-4-(1-(4- | 2 mg, 4% as a mixture of | ¹H NMR (400 MHz, CD₃OD) δ 8.30-8.15 (m, 1H), 7.93 (s, 1H), 6.46 (t, J = 10.7 Hz, |
| | fluoro-6-isopropoxypyridin-3- | diastereomers | 1H), 5.56 (s, 1H), 5.47 (s, 2H), 5.24 (s, 1H), 4.13-3.86 (m, 1H), 3.43 (s, 4H), 3.13 (s, |
| | yl)ethyl)piperazin-1-yl)-4-methyl-5- | | 1H), 2.92 (s, 1H), 2.69 (s, 1H), 2.52-2.37 (m, 1H), 1.98 (s, 1H), 1.65 (s, 2H), 1.53 (s, |
| | oxo-4,5-dihydro-2H-pyrazolo[4,3- | | 2H), 1.42-1.28 (m, 9H), 1.08-0.89 (m, 3H), 0.83-0.63 (m, 3H) ppm. MS: M/e 510 |
| | b]pyridin-2-yl)acetonitrile | | (M + 1)⁺. |
| A359 | 2-(7-((2S,5R)-2,5-diethyl-4-(1-(3- | 9 mg, 19% as a mixture of | ¹H NMR (400 MHz, CD₃OD) δ 8.25 (s, 1H), 7.92 (s, 1H), 7.45 (d, J = 10.7 Hz, 1H), |
| | fluoro-5-methylpyridin-2- | diastereomers | 5.53 (s, 1H), 5.47 (s, 2H), 4.49-4.12 (m, 2H), 3.57-3.39 (m, 4H), 3.18-3.01 (m, 1H), |
| | yl)ethyl)piperazin-1-yl)-4-methyl-5- | | 2.87 (d, J = 12.0 Hz, 1H), 2.38 (s, 4H), 1.90 (d, J = 6.8 Hz, 1H), 1.76-1.27 (m, 7H), |
| | oxo-4,5-dihydro-2H-pyrazolo[4,3- | | 0.95 (dt, J = 49.9, 6.8 Hz, 3H), 0.70 (dt, J = 52.2, 6.6 Hz, 3H) ppm. MS: M/e 466 |
| | b]pyridin-2-yl)acetonitrile | | (M + 1)⁺. |
| A360 | 2-(7-((2S,5R)-4-(1-(5- | separated isomer | A360a (the earlier peak): ¹H NMR (400 MHz, CD₃OD) δ 8.46 (s, 1H), 7.93 (s, 1H), |
| | chloropyridin-2-yl)ethyl)-2,5- | A360a(20 | 7.87 (d, J = 8.1 Hz, 1H), 7.66 (d, J = 8.3 Hz, 1H), 5.56 (s, 1H), 5.47 (s, 2H), 4.86-4.16 |
| | diethylpiperazin-1-yl)-4-methyl-5- | mg) | (m, 2H), 3.77 (s, 1H), 3.52 (d, J = 13.1 Hz, 1H), 3.43 (s, 3H), 3.12 (s, 1H), 2.76 (d, J = |
| | oxo-4,5-dihydro-2H-pyrazolo[4,3- | | 12.2 Hz, 1H), 2.24 (d, J = 12.3 Hz, 1H), 1.94 (s, 1H), 1.70 (s, 2H), 1.52 (s, 1H), 1.34 |
| | b]pyridin-2-yl)acetonitrile | | (d, J = 6.0 Hz, 3H), 1.03 (d, J = 7.0 Hz, 3H), 0.67 (s, 3H). MS: M/e 468 (M + 1)⁺. |
| | | separated isomer | A360b (the later peak): ¹H NMR (400 MHz, CD₃OD) δ 8.47 (s, 1H), 7.92 (s, 1H), 7.87 |
| | | A360b | (d, J = 8.3 Hz, 1H), 7.61 (d, J = 8.4 Hz, 1H), 5.56 (s, 1H), 5.46 (s, 2H), 4.58 (s, 2H), |
| | | (16 mg) | 3.95 (d, J = 6.8 Hz, 1H), 3.43 (s, 3H), 3.35 (s, 1H), 2.99-2.91 (m, 2H), 2.36 (s, 1H), |
| | | | 2.12-2.03 (m, 1H), 1.85-1.78 (m, 1H), 1.59-1.52 (m, 2H), 1.37 (d, J = 6.3 Hz, 3H), |
| | | | 0.94 (t, J = 6.9 Hz, 3H), 0.74 (t, J = 7.2 Hz, 3H). MS: M/e 468 (M + 1)⁺ |
| A361 | 2-(7-((2S,5R)-2,5-diethyl-4-(1-(5- | 23 mg | ¹H NMR (400 MHz, CD₃OD) δ 8.47 (s, 1H), 7.93 (d, J = 2.0 Hz, 1H), 7.84-7.70 (m, |
| | (trifluoromethoxy)pyridin-2- | | 2H), 5.56 (s, 1H), 5.47 (d, J = 3.2 Hz, 2H), 4.05-3.78 (m, 1H), 3.58-3.48 (m, 0.5H), |
| | yl)ethyl)piperazin-1-yl)-4-methyl-5- | | 3.43 (s, 3H), 3.39-3.34 (m, 0.5H), 3.33-3.31 (m, 2H), 3.19-3.10 (m, 0.5H), 3.03- |
| | oxo-4,5-dihydro-2H-pyrazolo[4,3- | | 2.92 (m, 1H), 2.81-2.74 (m, 0.5H), 2.41-2.33 (m, 0.5H), 2.27-2.18 (m, 0.5H), 2.15- |
| | b]pyridin-2-yl)acetonitrile | | 1.47 (m, 4H), 1.43-1.33 (m, 3H), 1.07-0.90 (m, 3H), 0.78-0.61 (m, 3H) ppm. |
| | | | MS: M/e 518 (M + 1)⁺. |
| A363 | 2-(7-((2S,5R)-4-(1-(5,6- | 7 mg | ¹H NMR (400 MHz, CD₃OD) δ 7.93 (d, J = 3.3 Hz, 1H), 7.57 (d, J = 7.9 Hz, 1H), 7.37 |
| | dimethylpyridin-2-yl)ethyl)-2,5- | | (dd, J = 14.4, 7.8 Hz, 1H), 5.56 (s, 1H), 5.47 (d, J = 3.4 Hz, 2H), 4.78-4.21 (m, 1H), |
| | diethylpiperazin-1-yl)-4-methyl-5- | | 3.84 (dd, J = 55.9, 6.6 Hz, 1H), 3.53 (d, J = 12.9 Hz, 1H), 3.43 (s, 3H), 3.34 (s, 1H), |
| | oxo-4,5-dihydro-2H-pyrazolo[4,3- | | 3.10 (dd, J = 55.7, 11.5 Hz, 1H), 2.85 (dd, J = 59.3, 11.9 Hz, 1H), 2.49 (s, 3H), 2.37 (d, |
| | b]pyridin-2-yl)acetonitrile | | J = 27.7 Hz, 1H), 2.31 (s, 3H), 2.19-1.92 (m, 1H), 1.91-1.51 (m, 3H), 1.34 (t, J = |
| | | | 6.7 Hz, 3H), 1.00 (dt, J = 30.6, 7.3 Hz, 3H), 0.77-0.60 (m, 3H) ppm. MS: M/e 462 |
| | | | (M + 1)⁺. |
| A364 | 2-(7-((2S,5R)-2,5-diethyl-4-(1-(6- | 5 mg as a mixture of | ¹H NMR (400 MHz, CD₃OD) δ 7.92 (d, J = 3.3 Hz, 1H), 7.47-7.38 (m, 1H), 6.94 (dd, |
| | methoxy-5-methylpyridin-2- | diastereomers | J = 41.6, 7.3 Hz, 1H), 5.54 (d, J = 5.5 Hz, 1H), 5.47 (d, J = 3.3 Hz, 2H), 4.75-4.25 |
| | yl)ethyl)piperazin-1-yl)-4-methyl-5- | | (m, 2H), 3.94 (s, 3H), 3.91-3.65 (m, 1H), 3.52 (d, J = 12.6 Hz, 1H), 3.43 (s, 3H), 3.19- |
| | oxo-4,5-dihydro-2H-pyrazolo[4,3- | | 2.44 (m, 3H), 2.15 (d, J = 4.9 Hz, 3H), 2.03-1.45 (m, 4H), 1.37 (dd, J = 25.5, 6.6 |
| | b]pyridin-2-yl)acetonitrile | | Hz, 3H), 0.97 (dt, J = 22.2, 7.5 Hz, 3H), 0.75 (dt, J = 22.0, 7.3 Hz, 3H) ppm. MS: M/e |
| | | | 478 (M + 1)⁺ |
| A365 | 2-(7-((2S,5R)-2,5-diethyl-4-(1-(6- | 21 mg, 16% as a mixture of | ¹H NMR (400 MHz, CD₃OD) δ 7.98-7.89 (m, 1H), 7.89-7.77 (m, 1H), 7.67-7.57 |
| | methylpyridazin-3- | diastereomers | (m, 1H), 5.56 (s, 1H), 5.52-5.42 (m, 2H), 4.72-3.48 (m, 3H), 3.43 (s, 3H), 3.30- |
| | yl)ethyl)piperazin-1-yl)-4-methyl-5- | | 3.27 (m, 1H), 3.21-2.75 (m, 2H), 2.73-2.62 (m, 3H), 2.36-2.13 (m, 1H), 2.12- |
| | oxo-4,5-dihydro-2H-pyrazolo[4,3- | | 1.51 (m, 4H), 1.46-1.35 (m, 3H), 1.07-0.90 (m, 3H), 0.80-0.58 (m, 3H). MS: M/e |
| | b]pyridin-2-yl)acetonitrile | | 449 (M + 1)⁺ |
| A366 | 2-(7-((2S,5R)-2,5-diethyl-4-(1-(3- | 2 mg, 2% as a mixture of | ¹H NMR (400 MHz, CD₃OD) δ 9.11 (d, J = 8.3 Hz, 1H), 8.03-7.82 (m, 4H), 7.65 (s, |
| | methylisoquinolin-7- | diastereomers | 1H), 5.55 (s, 1H), 5.46 (s, 2H), 4.05-3.80 (m, 1H), 3.55 (d, J = 12.4 Hz, 1H), 3.43 (s, |
| | yl)ethyl)piperazin-1-yl)-4-methyl-5- | | 3H), 3.23 (s, 1H), 3.09-2.90 (m, 1H), 2.67 (s, 4H), 2.37 (d, J = 11.3 Hz, 1H), 2.22- |
| | oxo-4,5-dihydro-2H-pyrazolo[4,3- | | 1.81 (m, 2H), 1.60 (s, 3H), 1.49-1.39 (m, 3H), 1.08-0.94 (m, 3H), 0.68-0.52 (m, 3H) |
| | b]pyridin-2-yl)acetonitrile | | ppm. MS: M/e 498 (M + 1)⁺. |
| A367 | 2-(7-((2S,5R)-2,5-diethyl-4-(1-(1- | 9 mg, 18% as a mixture of | ¹H NMR (400 MHz, CD₃OD) δ 8.24 (d, J = 7.9 Hz, 1H), 7.91 (dd, J = 15.9, 8.6 Hz, |
| | methylisoquinolin-3- | diastereomers | 2H), 7.85-7.73 (m, 2H), 7.67 (d, J = 7.5 Hz, 1H), 5.57 (s, 1H), 5.47 (d, J = 4.8 Hz, |
| | yl)ethyl)piperazin-1-yl)-4-methyl-5- | | 2H), 4.39-3.87 (m, 2H), 3.73-3.35 (m, 6H), 3.18-2.90 (m, 4H), 2.75-2.47 (m, 1H), |
| | oxo-4,5-dihydro-2H-pyrazolo[4,3- | | 2.13 (s, 1H), 1.92-1.44 (m, 6H), 1.28 (s, 1H), 1.14-0.94 (m, 3H), 0.77-0.60 (m, 3H) |
| | b]pyridin-2-yl)acetonitrile | | ppm. MS: M/e 498 (M + 1)⁺. |
| A369 | 2-(7-((2S,5R)-4-(4- | 21 mg, 73% | ¹H NMR (400 MHz, CD₃OD) δ 7.95 (s, 1H), 7.32 (t, J = 8.1 Hz, 2H), 7.20 (s, 2H), 5.63 |
| | cyclopropylbenzoyl)-2,5- | | (d, J = 12.9 Hz, 1H), 5.49 (s, 2H), 4.87-4.35 (m, 3H), 3.99-3.47 (m, 3H), 3.44 (s, |
| | diethylpiperazin-1-yl)-4-methyl-5- | | 3H), 1.99-1.60 (m, 5H), 1.03 (d, J = 7.4 Hz, 5H), 0.75 (dd, J = 35.3, 22.7 Hz, 5H). |
| | oxo-4,5-dihydro-2H-pyrazolo[4,3- | | MS: M/e 473 (M + 1)⁺. |
| | b]pyridin-2-yl)acetonitrile | | |
| A370 | 2-(7-((2S,5R)-2,5-diethyl-4-(5- | 22 mg, 81% | ¹H NMR (400 MHz, CD₃OD) δ 8.48 (s, 1H), 7.95 (d, J = 2.0 Hz, 1H), 7.80 (d, J = 8.0 |
| | methylpicolinoyl)piperazin-1-yl)-4- | | Hz, 1H), 7.52 (d, J = 7.9 Hz, 1H), 5.64 (d, J = 10.9 Hz, 1H), 5.49 (s, 2H), 4.78 (s, 2H), |
| | methyl-5-oxo-4,5-dihydro-2H- | | 4.60 (d, J = 13.7 Hz, 1H), 3.68 (dd, J = 17.9, 14.2 Hz, 1H), 3.60-3.51 (m, 1H), 3.45 |
| | pyrazolo[4,3-b]pyridin-2- | | (d, J = 1.8 Hz, 3H), 3.37 (d, J = 3.3 Hz, 1H), 2.43 (s, 3H), 1.94-1.64 (m, 4H), 1.08- |
| | yl)acetonitrile | | 0.96 (m, 3H), 0.82-0.64 (m, 3H). MS: M/e 448 (M + 1)⁺ |
| A371 | 2-(7-((2S,5R)-2,5-diethyl-4-(2,6- | 16 mg | ¹H NMR (400 MHz, CD₃OD) δ 9.49 (d, J = 9.8 Hz, 2H), 8.78 (d, J = 5.7 Hz, 1H), 8.31 |
| | naphthyridine-3-carbonyl)piperazin- | | (d, J = 5.1 Hz, 1H), 8.10 (d, J = 5.8 Hz, 1H), 7.96 (d, J = 2.9 Hz, 1H), 5.65 (d, J = 9.8 |
| | 1-yl)-4-methyl-5-oxo-4,5-dihydro- | | Hz, 1H), 5.49 (d, J = 2.7 Hz, 2H), 4.68 (d, J = 13.8 Hz, 1H), 4.63-4.29 (m, 1H), 4.00 |
| | 2H-pyrazolo[4,3-b]pyridin-2- | | (s, 1H), 3.84-3.56 (m, 2H), 3.45 (d, J = 2.7 Hz, 3H), 3.43-3.33 (m, 1H), 2.05-1.67 |
| | yl)acetonitrile | | (m, 4H), 1.06 (dt, J = 24.4, 7.2 Hz, 3H), 0.76 (dt, J = 52.8, 7.3 Hz, 3H) ppm. MS: M/e |
| | | | 485 (M + 1)⁺. |
| A372 | 2-(7-((2S,5R)-2,5-diethyl-4- | 55 mg | ¹H NMR (400 MHz, CD₃OD) δ 9.32 (s, 1H), 8.19 (d, J = 8.2 Hz, 1H), 8.10-8.03 (m, |
| | (isoquinoline-3-carbonyl)piperazin- | | 2H),7.95 (d, J = 3.1 Hz, 1H), 7.87 (t, J = 7.5 Hz, 1H), 7.78 (d, J = 7.4 Hz, 1H), 5.65 (d, |
| | 1-yl)-4-methyl-5-oxo-4,5-dihydro- | | J = 13.4 Hz, 1H), 5.49 (d, J = 2.7 Hz, 2H), 4.67 (d, J = 13.7 Hz, 1H), 4.62-4.20 (m, |
| | 2H-pyrazolo[4,3-b]pyridin-2- | | 1H), 4.01 (s, 1H), 3.82-3.53 (m, 2H), 3.45 (d, J = 3.0 Hz, 3H), 3.32 (s, 1H), 2.03- |
| | yl)acetonitrile | | 1.57 (m, 4H), 1.06 (dt, J = 27.0, 7.3 Hz, 3H), 0.72 (dt, J = 60.9, 7.4 Hz, 3H) ppm. MS: |
| | | | M/e 484 (M + 1)⁺. |
| A373 | 2-(7-((2S,5R)-2,5-diethyl-4- | 33 mg | ¹H NMR (400 MHz, CD₃OD) δ 8.96 (d, J = 4.3 Hz, 1H), 8.46 (d, J = 8.4 Hz, 1H), 8.10 |
| | (quinoline-7-carbonyl)piperazin-1- | | (dd, J = 15.0, 10.4 Hz, 2H), 7.95 (d, J = 3.7 Hz, 1H), 7.76-7.58 (m, 2H), 5.65 (d, J = |
| | yl)-4-methyl-5-oxo-4,5-dihydro-2H- | | 13.0 Hz, 1H), 5.49 (d, J = 2.6 Hz, 2H), 4.68 (d, J = 13.5 Hz, 1H), 4.63-4.09 (m, 1H), |
| | pyrazolo[4,3-b]pyridin-2- | | 3.76 (dd, J = 26.1, 15.5 Hz, 1H), 3.58 (d, J = 13.7 Hz, 2H), 3.44 (d, J = 3.6 Hz, 3H), |
| | yl)acetonitrile | | 3.35 (d, J = 32.6 Hz, 1H), 2.03-1.65 (m, 4H), 1.14-1.00 (m, 3H), 0.73 (dt, J = 69.7, |
| | | | 7.3 Hz, 3H) ppm. MS: M/e 484 (M + 1)⁺. |
| A374 | 2-(7-((2S,5R)-2,5-diethyl-4- | 40 mg | ¹H NMR (400 MHz, CD₃OD) δ 9.11 (d, J = 7.4 Hz, 1H), 8.23-8.11 (m, 2H), 8.02- |
| | (quinoxaline-2-carbonyl)piperazin- | | 7.89 (m, 3H), 5.67 (d, J = 7.2 Hz, 1H), 5.50 (s, 2H), 4.85-4.75 (m, 1H), 4.70 (d, J = |
| | 1-yl)-4-methyl-5-oxo-4,5-dihydro- | | 13.6 Hz, 1H), 4.63-4.36 (m, 1H), 4.31-3.94 (m, 1H), 3.81-3.49 (m, 2H), 3.45 (s, |
| | 2H-pyrazolo[4,3-b]pyridin-2- | | 3H), 2.10-1.73 (m, 4H), 1.06 (dt, J = 22.3, 7.3 Hz, 3H), 0.85 (dt, J = 45.4, 7.4 Hz, 3H) |
| | yl)acetonitrile | | ppm. MS: M/e 485 (M + 1)⁺. |
| A376 | 2-(7-((2S,5R)-2,5-diethyl-4-(1-(4- | 7 mg, 13% | ¹H NMR (400 MHz, CD₃OD) δ 7.93-7.89 (m, 1H), 7.32-7.24 (m, 2H), 6.94-6.82 |
| | ((tetrahydrofuran-3- | | (m, 2H), 5.54 (s, 1H), 5.46 (s, 2H), 4.01-3.45 (m, 8H), 3.43 (s, 3H), 3.35-3.31 (m, |
| | yl)methoxy)phenyl)ethyl)piperazin- | | 1H), 3.21-2.37 (m, 4H), 2.21-1.44 (m, 7H), 1.38-1.20 (m, 3H), 1.05-0.90 (m, |
| | 1-yl)-4-methyl-5-oxo-4,5-dihydro- | | 3H), 0.75-0.55 (m, 3H) ppm. MS: M/e 533 (M + 1)⁺. |
| | 2H-pyrazolo[4,3-b]pyridin-2- | | |
| | yl)acetonitrile | | |
| A377 | 2-(7-((2S,5R)-2,5-diethyl-4-(1-(4- | 5 mg, 11% | ¹H NMR (400 MHz, CD₃OD) δ 7.93-7.89 (m, 1H), 7.32-7.22 (m, 2H), 6.92-6.82 |
| | methoxyphenyl)ethyl)piperazin-1- | | (m, 2H), 5.54 (s, 1H), 5.46 (s, 2H), 3.81-3.75 (m, 3H), 3.71-3.45 (m, 2H), 3.43 (s, |
| | yl)-4-methyl-5-oxo-4,5-dihydro-2H- | | 3H), 3.35-3.31 (m, 1H), 3.21-2.79 (m, 2H), 2.65-2.31 (m, 2H), 2.15-1.45 (m, 4H), |
| | pyrazolo[4,3-b]pyridin-2- | | 1.37-1.24 (m, 3H), 1.05-0.90 (m, 3H), 0.75-0.55 (m, 3H) ppm. MS: M/e 463 |
| | yl)acetonitrile | | (M + 1)⁺ |
| A378 | 2-(7-((2S,5R)-2,5-diethyl-4-(1-(4- | 4 mg, 8% | ¹H NMR (400 MHz, CD₃OD) δ 7.93-7.90 (m, 1H), 7.33-7.22 (m, 2H), 6.78-6.68 |
| | (oxetan-3- | | (m, 2H), 5.54 (s, 1H), 5.46 (s, 2H), 5.31-5.20 (m, 1H), 5.06-4.97 (m, 2H), 4.72- |
| | yloxy)phenyl)ethyl)piperazin-1-yl)- | | 4.62 (m, 2H), 3.81-3.45 (m, 4H), 3.43 (s, 3H), 3.35-3.31 (m, 1H), 3.16-2.80 (m, |
| | 4-methyl-5-oxo-4,5-dihydro-2H- | | 1H), 2.65-2.35 (m, 1H), 2.15-1.45 (m, 4H), 1.36-1.24 (m, 3H), 1.05-0.90 (m, |
| | pyrazolo[4,3-b]pyridin-2- | | 3H), 0.75-0.55 (m, 3H) ppm. MS: M/e 505 (M + 1)⁺ |
| | yl)acetonitrile | | |
| A379 | 2-(7-((2S,5R)-2,5-diethyl-4-(1-(4- | 1 mg, 2% | ¹H NMR (400 MHz, CD₃OD) δ 7.93-7.89 (m, 1H), 7.34-7.22 (m, 2H), 6.97-6.87 |
| | (oxetan-3- | | (m, 2H), 5.54 (s, 1H), 5.46 (s, 2H), 4.65-4.54 (m, 2H), 4.20 (t, J = 4.2 Hz, 2H), 3.78- |
| | ylmethoxy)phenyl)ethyl)piperazin- | | 3.45 (m, 4H), 3.43 (s, 3H), 3.35-3.31 (m, 1H), 3.19-2.79 (m, 2H), 2.65-2.31 (m, |
| | 1-yl)-4-methyl-5-oxo-4,5-dihydro- | | 2H), 2.19-1.44 (m, 5H), 1.36-1.24 (m, 3H), 1.07-0.90 (m, 3H), 0.75-0.55 (m, |
| | 2H-pyrazolo[4,3-b]pyridin-2- | | 3H) ppm. MS: M/e 519 (M + 1)⁺. |
| | yl)acetonitrile | | |
| A380 | 2-(7-((2S,5R)-2,5-diethyl-4-(1-(4- | 10 mg, 12% | ¹H NMR (400 MHz, CD₃OD) δ 7.92 (s, 1H), 7.35-7.25 (m, 2H), 6.97-6.88 (m, 2H), |
| | ((tetrahydro-2H-pyran-4- | | 5.54 (s, 1H), 5.46 (s, 2H), 4.60-4.45 (m, 1H), 4.01-3.88 (m, 2H), 3.80-3.45 (m, |
| | yl)oxy)phenyl)ethyl)piperazin-1-yl)- | | 4H), 3.43 (s, 3H), 3.35-3.31 (m, 1H), 3.21-2.79 (m, 2H), 2.66-2.34 (m, 2H), 2.14- |
| | 4-methyl-5-oxo-4,5-dihydro-2H- | | 1.90 (m, 3H), 1.85-1.45 (m, 5H), 1.38-1.26 (m, 3H), 1.05-0.90 (m, 3H), 0.75- |
| | pyrazolo[4,3-b]pyridin-2- | | 0.55 (m, 3H) ppm. MS: M/e 533 (M + 1)⁺. |
| | yl)acetonitrile | | |
| A382 | 2-(7-((2S,5R)-4-(1-(4-(4,4- | 5 mg as a mixture of | ¹H NMR (400 MHz, CD₃OD) δ 7.92 (d, J = 1.6 Hz, 1H), 7.57-7.49 (m, 2H), 7.48- |
| | difluoropiperidine-1- | diastereomers | 7.41 (m, 2H), 5.55 (d, J = 3.2 Hz, 1H), 5.46 (d, J = 3.6 Hz, 2H), 3.94-3.45 (m, 5H), |
| | carbonyl)phenyl)ethyl)-2,5- | | 3.43 (s, 3H), 3.30-3.25 (m, 2H), 3.20-2.85 (m, 2H), 2.73-2.64 (m, 1H), 2.51-2.33 |
| | diethylpiperazin-1-yl)-4-methyl-5- | | (m, 1H), 2.17-1.51 (m, 8H), 1.35 (dd, J = 13.6, 6.4 Hz, 3H), 0.99 (dt, J = 24.8, 7.2 |
| | oxo-4,5-dihydro-2H-pyrazolo[4,3- | | Hz, 3H), 0.66 (dt, J = 33.6, 7.2 Hz, 3H) ppm. MS: M/e 580 (M + 1)⁺. |
| | b]pyridin-2-yl)acetonitrile | | |
| A384 | 2-(7-((2S,5R)-4-(1-(3,4- | 30 mg, 43% as a mixture of | ¹H NMR (400 MHz, CD₃OD) δ 7.92 (d, J = 2.3 Hz, 1H), 7.17-7.03 (m, 3H), 5.54 (s, |
| | dimethylphenyl)ethyl)-2,5- | diastereomers | 1H), 5.46 (d, J = 3.2 Hz, 2H), 4.86-3.83 (m, 2H), 3.72-3.45 (m, 2H), 3.43 (s, 3H), |
| | diethylpiperazin-1-yl)-4-methyl-5- | | 3.28-3.11 (m, 1H), 3.01-2.83 (m, 1H), 2.62-2.44 (m, 1H), 2.25 (t, J = 6.0 Hz, 6H), |
| | oxo-4,5-dihydro-2H-pyrazolo[4,3- | | 2.15-1.98 (m, 1H), 1.87-1.55 (m, 2H), 1.53-1.46 (m, 1H), 1.30 (dd, J = 16.6, 6.5 |
| | b]pyridin-2-yl)acetonitrile | | Hz, 3H), 0.98 (dt, J = 24.1, 7.5 Hz, 3H), 0.65 (dt, J = 18.2, 7.3 Hz, 3H). MS: M/e 461 |
| | | | (M + 1)⁺. |
| A385 | 2-(7-((2S,5R)-4-(1-(4,5- | 2 mg, 4% as a mixture of | ¹H NMR (400 MHz, CD₃OD) δ 8.20 (s, 1H), 7.93 (d, J = 3.7 Hz, 1H), 7.40 (m, 1H), |
| | dimethylpyridin-2-yl)ethyl)-2,5- | diastereomers | 5.56 (s, 1H), 5.47 (d, J = 3.6 Hz, 2H), 3.91 (s, 1H), 3.59-3.34 (m, 4H), 3.29-3.11 (m, |
| | diethylpiperazin-1-yl)-4-methyl-5- | | 2H), 3.05-2.92 (m, 1H), 2.86-2.69 (m, 1H), 2.35 (d, J = 7.0 Hz, 3H), 2.29 (d, J = 2.9 |
| | oxo-4,5-dihydro-2H-pyrazolo[4,3- | | Hz, 3H), 2.08 (s, 1H), 1.89-1.50 (m, 4H), 1.42-1.32 (m, 3H), 1.00 (m , 3H), 0.69 |
| | b]pyridin-2-yl)acetonitrile | | (m, 3H) ppm. MS: M/e 462 (M + 1)⁺ |
| A386 | 2-(6-(1-((2R,5S)-4-(2- | 13 mg, 24% as a mixture of | ¹H NMR (400 MHz, CD₃OD) δ 8.66 (s, 1H), 8.02 (d, J = 8.4 Hz, 1H), 7.93 (d, J = 3.2 |
| | (cyanomethyl)-4-methyl-5-oxo-4,5- | diastereomers | Hz, 1H), 7.74-7.65 (m, 1H), 5.57 (s, 1H), 5.47 (d, J = 3.8 Hz, 2H), 4.07-3.81 (m, 1H), |
| | dihydro-2H-pyrazolo[4,3-b]pyridin- | | 3.61-3.34 (m, 5H), 3.23-2.75 (m, 2H), 2.47-2.20 (m, 1H), 2.17-1.92 (m, 1H), 1.90- |
| | 7-yl)-2,5-diethylpiperazin-1- | | 1.64 (m, 8H), 1.63-1.51 (m, 2H), 1.45-1.35 (m, 3H), 1.08-0.94 (m, 3H), 0.78-0.62 |
| | yl)ethyl)pyridin-3-yl)-2- | | (m, 3H) ppm. MS: M/e 501 (M + 1)⁺. |
| | methylpropanenitrile | | |
| A387 | 2-(7-((2S,5R)-2,5-diethyl-4-(1-(3- | 2 mg, 4% as a mixture of | ¹H NMR (400 MHz, CD₃OD) δ 8.04 (d, J = 17.5 Hz, 1H), 7.93 (d, J = 7.3 Hz, 1H), 7.43- |
| | fluoro-5-isopropoxypyridin-2- | diastereomers | 7.29 (m, 1H), 5.54 (d, J = 6.1 Hz, 1H), 5.47 (d, J = 5.5 Hz, 2H), 4.70 (dd, J = 11.4, |
| | yl)ethyl)piperazin-1-yl)-4-methyl-5- | | 5.6 Hz, 1H), 4.48 (s, 1H), 3.66-3.36 (m, 4H), 3.31 (s, 2H), 3.22-2.85 (m, 2H), 2.55 (s, |
| | oxo-4,5-dihydro-2H-pyrazolo[4,3- | | 1H), 2.05-1.80 (m, 1H), 1.78-1.50 (m, 3H), 1.39 (dd, J = 15.2, 5.0 Hz, 9H), 1.09-0.84 |
| | b]pyridin-2-yl)acetonitrile | | (m, 3H), 0.82-0.68 (m, 3H) ppm. MS: M/e 510 (M + 1)⁺. |
| A388 | 2-(7-((2S,5R)-2,5-diethyl-4-(1- | 24 mg as a mixture of | ¹H NMR (400 MHz, CD₃OD) δ 8.43 (d, J = 6.8 Hz, 1H), 7.93 (d, J = 2.1 Hz, 1H), 7.83 |
| | (imidazo[1,2-a]pyridin-2- | diastereomers | (d, J = 10.9 Hz, 1H), 7.58-7.51 (m, 1H), 7.40-7.32 (m, 1H), 6.95 (t, J = 6.8 Hz, 1H), |
| | yl)ethyl)piperazin-1-yl)-4-methyl-5- | | 5.55 (d, J = 3.4 Hz, 1H), 5.47 (d, J = 3.3 Hz, 2H), 4.07 (dd, J = 62.3, 6.7 Hz, 1H), 3.57- |
| | oxo-4,5-dihydro-2H-pyrazolo[4,3- | | 3.45 (m, 1H), 3.43 (s, 3H), 3.38 (s, 1H), 3.20-3.02 (m, 1H), 2.96-2.82 (m, 1H), |
| | b]pyridin-2-yl)acetonitrile | | 2.79-2.64 (m, 1H), 2.20-1.59 (m, 4H), 1.52 (dd, J = 24.9, 6.6 Hz, 3H), 1.29 (s, 1H), |
| | | | 1.05-0.91 (m, 3H), 0.86-0.68 (m, 3H). MS: M/e 473 (M + 1)⁺. |
| A389 | 2-(7-((2S,5R)-2,5-diethyl-4-(1- | 0.7 mg, 1.48% as a mixture of | ¹H NMR (400 MHz, CD₃OD) δ 8.44 (m, 1H), 7.92 (d, J = 2.5 Hz, 1H), 7.59 (t, J = 9.7 |
| | (pyrazolo[1,5-a]pyridin-2- | diastereomers | Hz, 1H), 7.19 (dd, J = 14.7, 5.9 Hz, 1H), 6.83 (q, J = 6.6 Hz, 1H), 6.56 (s, 1H), 5.54 (d, |
| | yl)ethyl)piperazin-1-yl)-4-methyl-5- | | J = 5.0 Hz, 1H), 5.47 (d, J = 3.3 Hz, 2H), 4.60 (s, 1H), 4.11-3.94 (m, 1H), 3.50 (d, J = |
| | oxo-4,5-dihydro-2H-pyrazolo[4,3- | | 14.5 Hz, 1H), 3.43 (s, 3H), 3.14 (d, J = 10.0 Hz, 1H), 2.97 (m, 1H), 2.76 (m, 1H), 2.62- |
| | b]pyridin-2-yl)acetonitrile | | 2.52 (m, 1H), 2.00 (m, 2H), 1.81 (m, 1H), 1.59 (m, 1H), 1.51 (d, J = 6.7 Hz, 2H), 1.45 |
| | | | (d, J = 6.7 Hz, 1H), 1.01 (t, J = 7.4 Hz, 2H), 0.95 (t, J = 7.5 Hz, 1H), 0.77 (t, J = 7.4 |
| | | | Hz, 1H), 0.68 (t, J = 7.5 Hz, 2H) ppm. MS: M/e 473 (M + 1)⁺. |
| A391 | 2-(but-2-yn-1-yl)-7-((2S,5R)-4-(1- | 28 mg, 40% as a mixture of | ¹H NMR (400 MHz, CD₃OD) δ 7.90 (s, 1H), 7.29 (s, 1H), 7.22-7.08 (m, 2H), 5.53 (s, |
| | (2,2-difluorobenzo[d][1,3]dioxol-5- | diastereomers | 1H), 5.04 (s, 2H), 4.72-4.30 (m, 1H), 3.75-3.38 (m, 6H), 3.11-2.09 (m, 3H), 1.86 |
| | yl)ethyl)-2,5-dimethylpiperazin-1- | | (s, 3H), 1.40-1.19 (m, 6H), 1.16-0.97 (m, 3H). MS: M/e 498 (M + 1)⁺. |
| | yl)-4-methyl-2,4-dihydro-5H- | | |
| | pyrazolo[4,3-b]pyridin-5-one | | |
| A395 | 2-(but-2-yn-1-yl)-7-((2S,5R)-5- | 8 mg as a mixture of | ¹H NMR (400 MHz, CD₃OD) δ 8.83 (t, J = 4.6 Hz, 1H), 8.37 (t, J = 8.2 Hz, 1H), 8.04- |
| | ethyl-2-methyl-4-(1-(quinolin-7- | diastereomers | 7.84 (m, 3H), 7.78 (t, J = 8.0 Hz, 1H), 7.52 (td, J = 8.1, 4.4 Hz, 1H), 5.54 (d, J = 6.9 |
| | yl)ethyl)piperazin-1-yl)-4-methyl- | | Hz, 1H), 5.02 (dd, J = 13.0, 2.2 Hz, 2H), 4.71-4.49 (m, 2H), , 3.95 (dd, J = 58.3, 6.4 |
| | 2,4-dihydro-5H-pyrazolo[4,3- | | Hz, 1H), 3.57 (d, J = 11.8 Hz, 1H), 3.44 (s, 3H), 3.23-2.77 (m, 2H), 2.51-2.20 (m, |
| | b]pyridin-5-one | | 1H), 1.84 (d, J = 10.2 Hz, 3H), 1.77-1.56 (m, 2H), 1.49-1.16 (m, 6H), 1.05-0.69 (m, |
| | | | 3H) ppm. MS: M/e 483 (M + 1)⁺. |
| A392 | 2-(but-2-yn-1-yl)-7-((2S,5R)-4-(1- | separated isomer | A392a (the earlier peak): ¹H NMR (400 MHz, CD₃OD) δ 7.89 (s, 1H), 7.28 (s, 1H), |
| | (2,2-difluorobenzo[d][1,3]dioxol-5- | A392a(28 | 7.21-7.08 (m, 2H), 5.55 (s, 1H), 5.08-4.97 (m, 2H), 4.86-4.32 (m, 2H), 3.80 (q, J = |
| | yl)ethyl)-5-ethyl-2-methylpiperazin- | mg) | 6.4 Hz, 1H), 3.44 (s, 3H), 3.30-3.23 (m, 1H), 2.95 (dd, J = 11.6, 4.0 Hz, 1H), 2.83 |
| | 1-yl)-4-methyl-2,4-dihydro-5H- | | (dd, J = 11.6, 2.0 Hz, 1H), 2.43 (d, J = 8.4 Hz, 1H), 1.85 (t, J = 2.4 Hz, 3H), 1.65-1.44 |
| | pyrazolo[4,3-b]pyridine-5-one | | (m, 2H), 1.38 (d, J = 6.4 Hz, 3H), 1.33 (d, J = 6.4 Hz, 3H), 0.77 (t, J = 7.6 Hz, 3H). |
| | | | MS: M/e 512 (M + 1)⁺. |
| | | separated isomer | A392b (the later peak): ¹H NMR (400 MHz, CD₃OD) δ 7.90 (s, 1H), 7.31 (s, 1H), 7.18 |
| | | A392b(29 | (d, J = 8.4 Hz, 1H), 7.11 (d, J = 8.4 Hz, 1H), 5.54 (s, 1H), 5.22-4.88 (m, 3H), 4.71- |
| | | mg) | 4.22 (m, 1H), 3.64 (q, J = 6.4 Hz, 1H), 3.50 (d, J = 13.6 Hz, 1H), 3.44 (s, 3H), 3.12 (d, |
| | | | J = 10.0 Hz, 1H), 2.73 (dd, J = 12.0, 4.0 Hz, 1H), 2.23 (d, J = 12.4 Hz, 1H), 1.86 (t, J = |
| | | | 2.4 Hz, 3H), 1.77-1.60 (m, 1H), 1.59-1.44 (m, 1H), 1.31 (d, J = 6.4 Hz, 3H), 1.21 |
| | | | (d, J = 6.8 Hz, 3H), 1.04 (t, J = 7.2 Hz, 3H). MS: M/e 512 (M + 1)⁺. |

The following compounds can also be prepared by the similar procedures disclosed herein.

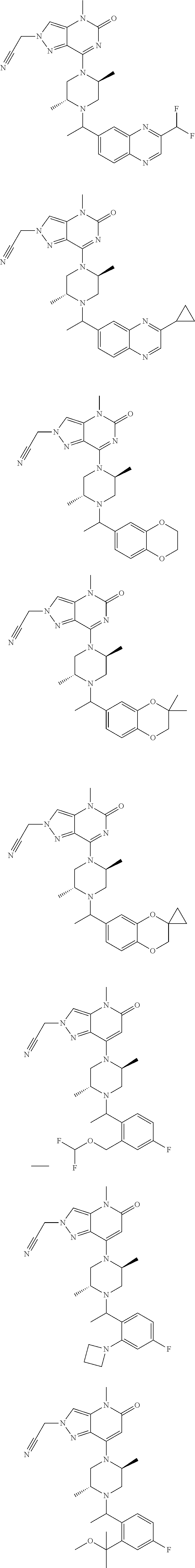

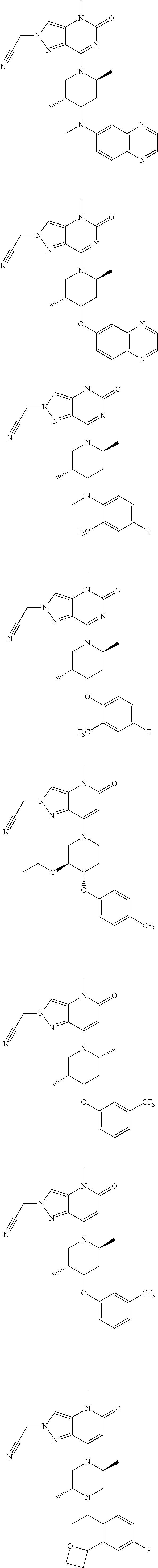

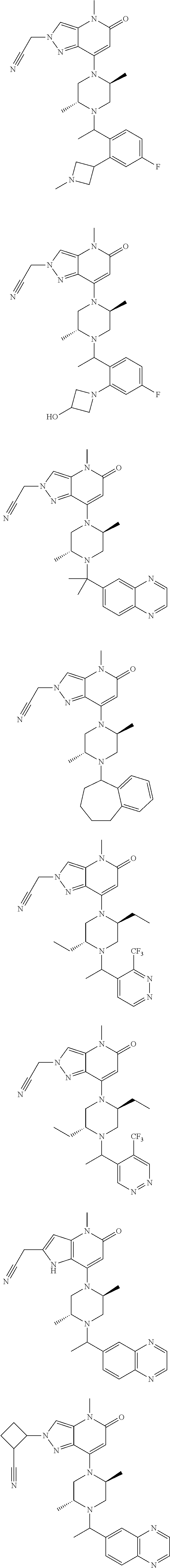

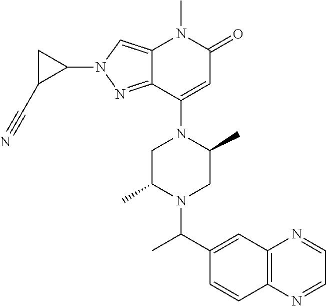 [SMILES: CC(c1ccc2nccnc2c1)N1C[C@H](C)N(c2cc(=O)n(C)c3cn(C4CC4C#N)nc23)C[C@H]1C]

### Assay

### 1) Biochemical DGK IC₅₀ Assays

Enzymatic reactions of DGKζ, DGKα and DGKδ were performed using ADP-Glo assay with lipid micelle substrate. Full length DGKζ (in-house protein M1-V929 with SEQ ID No: 2) was expressed in baculovirus expression system. Full length DGKα (D21-10BG, SignalChem) and full length DGKδ (D23-10G, SignalChem) were purchased. Lipid micelle was prepared by dissolving DAG (Sigma, 317505-10MG) and PS (Sigma, P7769-100MG) with chloroform which was furtherly removed by rotary evaporation. The resulted product was resuspended in buffer containing 25 mM HEPES pH 7.0, 0.5 mM EDTA and 160 mM Octyl β-D glucopyranoside by vigorous mixing and ultrasonic (IID, Scientz)

| | Final concentration in reaction | | |
|---|---|---|---|
| Enzyme | Enzyme (nM) | ATP (μM) | DAG (μM) |
| DGKα | 7.5 | 140 | 80 |
| DGKδ | 10 | 140 | 80 |
| DGKζ | 0.5 | 140 | 80 |

The inhibition activities testing for the compound disclosed herein were carried out at room temperature in assay buffer containing 50 mM HEPES, 10 mM MgCl₂, 0.01% BSA, 0.1 mM Na₃VO₄, 0.005% Tween-20 and 0.01 mM CaCl₂. Compounds in DMSO were dispensed into wells of a black 384 well plate (Corning 4514) using D300e digital dispenser (Tecan). The ranges of compounds final concentration were 1.55-10000 nM or 23.3-150000 nM. 3 μL 2× enzyme solution was added to wells. After incubation for 1 hour, 3 μL 2× substrates solution containing 160 μM DAG and 280 μM ATP was added to the wells to initiate reaction. After 1 hour reaction, 5 μL ADP-Glo reagent (Promga V9101) was added and incubated for 40 minutes. 10 μL Kinase Detection reagent was added and incubated for 30 minutes. Luminescence was measured on a microplate reader (PHERAstar FSX, BMG labtech). The IC₅₀s are calculated based on inhibition of enzyme activity in the presence of increasing concentrations of compounds. Selected compounds had no inhibitory activity on DGKδ. IC₅₀s of the compounds disclosed herein for DGKζ and DGKα are shown in Table 1.

### Baculovirus Expression of Human DGKζ

Human His-TEV-DGK-zeta-pFastBac1 and human baculovirus samples was generated using the Bac-to-Bac baculovirus expression system (Invitrogen) according to the manufacturer's protocol. The DNA used for expression of DGK-zeta, have SEQ ID Nos: 1. Baculovirus amplification was achieved using infected SF9 cells at 1:2000 virus/cells ratios, and grown for 96 hours at 27° C. post-transfected.

The expression scaled up for each protein was carried out in the flask 3 L from CORNING. 4 L of 3×10⁶ cells/mL Sf9 cells (Expression System, Invitrogen) grown in SF900™ II SFM insect medium (Expression System) was infected with virus stock at 1:200 virus/cells ratio, and grown for 48 hours at 27° C. post-transfection. The infected cell culture was harvested by centrifugation at 6000 rpm for 15 min 4° C. in a SORVALL LYNX6000 centrifuge. The cell pellets were stored at −80° C.

### Purification of Human DGK-Zeta

Full length human DGK produced as described above, was purified from Sf9 baculovirus-infected insect cell paste. The cells were lysed using sonication method, and the lysates were clarified by centrifugation. The clarified lysates were purified to ˜90% homogeneity, using two successive column chromatography steps on an AKTA Purifier system. The two steps column chromatography included nickel affinity resin capture (i.e. Ni-NTA Agarose, Qiagen), followed by size exclusion chromatography (i.e. Hiload16/60 Superdex200 prep grade, GE Healthcare. The protein was delivered and stored at −80° C. The formulation buffers were identical for the protein: 25 mM Tris, 150 mmol/L NaCl, 2 mM DTT, pH8.0.

SEQ ID No: 1 is Nucleotide sequence encoding His-TEV-hDGKζ-(M1-V929):
ATGCATCACCATCACCATCACCATCACGAGAACCTGTACTTCCAGGGATCCATGGAACCCCG
TGATGGTAGCCCCGAAGCTCGTAGCTCCGATTCCGAGTCCGCCAGCGCTTCCTCCTCCGGTAGCG
AACGTGACGCTGGTCCCGAGCCCGACAAAGCTCCCCGTCGTCTGAATAAGCGCCGTTTTCCCGG
TCTCCGTCTGTTCGGCCACCGCAAGGCCATCACTAAGTCCGGTCTCCAGCATCTGGCTCCTCCTC
CTCCTACCCCCGGTGCTCCTTGCTCCGAATCCGAGCGCCAGATTCGCTCCACTGTGGATTGGTCC
GAAAGCGCCACCTATGGTGAGCATATCTGGTTCGAGACCAACGTCTCCGGCGACTTCTGTTATGT
CGGTGAGCAATACTGTGTGGCTCGTATGCTGCAGAAGTCCGTGTCCCGCCGTAAATGCGCCGCTT
GCAAAATCGTGGTCCATACCCCTTGCATCGAGCAACTGGAGAAAATCAACTTCCGCTGCAAGCC
CAGCTTTCGTGAGTCCGGTTCCCGCAACGTGCGCGAACCTACTTTCGTGCGCCACCACTGGGTG
CATCGTCGTCGCCAAGACGGCAAATGCCGCCACTGCGGCAAAGGTTTTCAGCAGAAATTCACCT
TCCACAGCAAGGAGATCGTCGCCATCAGCTGCAGCTGGTGCAAACAAGCTTACCATTCCAAAGT
GAGCTGCTTCATGCTCCAGCAGATCGAAGAGCCTTGCTCTCTGGGTGTGCATGCTGCTGTCGTGA
TTCCCCCTACTTGGATTCTGCGTGCTCGCCGTCCCCAGAACACTCTGAAGGCCTCCAAAAAGAA
GAAGCGCGCCAGCTTCAAGCGTAAGAGCTCCAAAAAGGGTCCCGAAGAGGGCCGTTGGCGTCC
CTTCATCATCCGCCCTACTCCTTCCCCTCTGATGAAGCCTCTGCTGGTCTTCGTCAACCCTAAGAG
CGGCGGCAACCAAGGTGCTAAAATCATCCAGTCCTTCCTCTGGTATCTGAACCCTCGTCAAGTGT
TCGACCTCAGCCAAGGCGGTCCTAAGGAGGCTCTGGAGATGTACCGCAAGGTCCACAATCTGCG
CATCCTCGCTTGTGGTGGCGATGGCACCGTGGGCTGGATTCTGTCCACTCTGGACCAACTGCGTC
TGAAACCTCCCCCCCCCGTGGCTATTCTGCCTCTCGGTACCGGCAACGATCTGGCTCGTACTCTG
AATTGGGGTGGTGGCTACACCGATGAGCCCGTGTCCAAGATTCTGTCCCACGTCGAAGAAGGCA
ATGTCGTCCAACTGGACCGTTGGGACCTCCACGCCGAACCCAACCCCGAGGCTGGCCCCGAGG
ACCGTGACGAGGGCGCTACTGACCGTCTGCCCCTCGACGTCTTCAATAATTACTTCTCTCTGGGC
TTTGACGCTCACGTGACTCTGGAATTTCATGAAAGCCGCGAGGCCAACCCCGAGAAGTTCAATT
CCCGTTTCCGCAACAAGATGTTCTACGCTGGCACCGCCTTCAGCGACTTCCTCATGGGCTCCAGC
AAGGACCTCGCTAAGCATATCCGCGTGGTGTGCGATGGCATGGATCTGACCCCTAAGATCCAAGA
TCTGAAGCCCCAATGTGTCGTGTTTCTGAACATCCCCCGCTACTGCGCTGGTACTATGCCTTGGG
GCCATCCCGGTGAACACCATGACTTCGAACCTCAGCGTCATGATGACGGCTATCTGGAGGTGATC
GGTTTCACCATGACCTCCCTCGCTGCTCTGCAAGTGGGTGGCCACGGCGAACGTCTGACTCAAT
GCCGCGAGGTGGTGCTGACCACCAGCAAAGCCATCCCCGTCCAAGTGGATGGTGAGCCTTGCAA
GCTGGCCGCCTCCCGTATCCGTATCGCTCTCCGCAATCAAGCTACCATGGTCCAGAAGGCCAAAC
GCCGCAGCGCTGCTCCTCTCCACAGCGACCAACAACCCGTCCCCGAACAGCTGCGCATCCAAGT
GTCCCGTGTCAGCATGCATGACTACGAGGCTCTGCACTACGACAAGGAACAGCTGAAGGAAGCC
AGCGTGCCTCTGGGTACTGTGGTCGTGCCCGGTGACAGCGATCTGGAGCTCTGCCGTGCCCACA
TCGAGCGTCTGCAGCAAGAGCCCGACGGTGCTGGTGCCAAGAGCCCTACTTGCCAAAAACTCTC
CCCCAAGTGGTGTTTCCTCGACGCTACCACCGCCAGCCGCTTCTACCGCATTGATCGCGCCCAAG
AGCATCTGAACTATGTCACCGAGATCGCTCAAGACGAGATCTACATCCTCGACCCCGAACTCCTC
GGTGCTAGCGCCCGTCCCGACCTCCCCACTCCTACCTCCCCTCTGCCCACTTCCCCTTGTTCCCCC
ACCCCTCGTAGCCTCCAAGGTGATGCTGCCCCTCCTCAAGGTGAGGAGCTCATTGAGGCCGCTA
AGCGTAACGATTTCTGCAAGCTCCAAGAGCTGCATCGTGCTGGTGGCGACCTCATGCACCGCGA
TGAGCAGAGCCGCACTCTGCTGCACCACGCTGTGTCCACTGGTAGCAAGGACGTGGTGCGCTAT
CTGCTGGACCACGCTCCTCCCGAGATCCTCGACGCTGTGGAAGAAAACGGCGAGACTTGCCTCC
ACCAAGCTGCTGCTCTGGGTCAACGTACCATCTGCCACTACATCGTCGAAGCTGGTGCTTCTCTG
ATGAAGACCGACCAGCAAGGTGATACTCCCCGTCAGCGCGCCGAGAAAGCCCAAGACACCGAA
CTGGCTGCCTATCTGGAGAACCGTCAGCACTACCAGATGATTCAGCGTGAAGACCAAGAGACCG
CCGTGTAA
SEQ ID No: 2 is Amino acid sequence of His-TEV-hDGKζ-(M1-V929):
MEPRDGSPEARSSDSESASASSSGSERDAGPEPDKAPRRLNKRRFPGLRLFGHRKAITKSGLQH
LAPPPPTPGAPCSESERQIRSTVDWSESATYGEHIWFETNVSGDFCYVGEQYCVARMLQKSVSRRKC
AACKIVVHTPCIEQLEKINFRCKPSFRESGSRNVREPTFVRHHWVHRRRQDGKCRHCGKGFQQKFTF
HSKEIVAISCSWCKQAYHSKVSCFMLQQIEEPCSLGVHAAVVIPPTWILRARRPQNTLKASKKKKRA
SFKRKSSKKGPEEGRWRPFIIRPTPSPLMKPLLVFVNPKSGGNQGAKIIQSFLWYLNPRQVEDLSQGG
PKEALEMYRKVHNLRILACGGDGTVGWILSTLDQLRLKPPPPVAILPLGTGNDLARTLNWGGGYTD
EPVSKILSHVEEGNVVQLDRWDLHAEPNPEAGPEDRDEGATDRLPLDVFNNYFSLGFDAHVTLEFH
ESREANPEKFNSRFRNKMFYAGTAFSDFLMGSSKDLAKHIRVVCDGMDLTPKIQDLKPQCVVFLNIP
RYCAGTMPWGHPGEHHDFEPQRHDDGYLEVIGFTMTSLAALQVGGHGERLTQCREVVLTTSKAIPV
QVDGEPCKLAASRIRIALRNQATMVQKAKRRSAAPLHSDQQPVPEQLRIQVSRVSMHDYEALHYDK
EQLKEASVPLGTVVVPGDSDLELCRAHIERLQQEPDGAGAKSPTCQKLSPKWCFLDATTASRFYRID
RAQEHLNYVTEIAQDEIYILDPELLGASARPDLPTPTSPLPTSPCSPTPRSLQGDAAPPQGEELIEAAK
RNDFCKLQELHRAGGDLMHRDEQSRTLLHHAVSTGSKDVVRYLLDHAPPEILDAVEENGETCLHQA
AALGQRTICHYIVEAGASLMKTDQQGDTPRQRAEKAQDTELAAYLENRQHYQMIQREDQETAV*

TABLE 1

| | DGKζ ADP-Glo assay | DGKα ADP-Glo assay |
|---|---|---|
| Compound | IC₅₀(nM) | IC₅₀(nM) |
| A1 | >150000 | 1800 |
| A2 | 71 | 7700 |
| A2a | 52 | 4800 |
| A2b | 13000 | 19000 |
| A3 | 3800 | 3200 |
| A4 | 36000 | 51000 |
| A5 | 3500 | 21000 |
| A6 | >150000 | >150000 |
| A7 | 1030 | >10000 |
| A8 | 570 | 31000 |
| A9 | 30000 | 27000 |
| A10 | 18000 | 110000 |
| A11 | >150000 | >150000 |
| A12 | 2200 | >150000 |
| A13 | 3700 | 5900 |
| A14 | 12000 | 52000 |
| A15 | 34000 | 5900 |
| A16 | 4200 | 9600 |
| A17 | 13000 | 9000 |
| A18 | 3700 | 5700 |
| A19 | 24000 | >150000 |
| A20 | 39000 | >150000 |
| A21 | 310 | 5300 |
| A22 | 94 | 200 |
| A22a | 13000 | 1100 |
| A22b | 72 | 190 |
| A23 | 57 | 120 |
| A24 | 4300 | 150 |
| A25 | 510 | 54 |
| A26 | 98000 | 4.3 |
| A27 | >10000 | 4.3 |
| A28 | 6.7 | 310 |
| A28a | 7.6 | 490 |
| A28b | 463 | 983 |
| A29 | 110 | 23000 |
| A29a | 2220 | >10000 |
| A29b | 57 | 4060 |
| A30 | 110 | 5100 |
| A31 | 8100 | 1200 |
| A32 | 230 | 1200 |
| A33 | >10000 | 160 |
| A34 | 5800 | 8300 |
| A35 | 1200 | 870 |
| A36 | 970 | 550 |
| A37 | 5400 | 140 |
| A38 | 610 | 899 |
| A39 | 1000 | 510 |
| A40 | 270 | 2300 |
| A41 | 1000 | >10000 |
| A42 | 3390 | >10000 |
| A43 | 65 | 2900 |
| A43a | 5080 | 37700 |
| A43b | 50 | 6570 |
| A44 | 610 | 8100 |
| A45 | 1420 | >10000 |
| A46a | >10000 | >10000 |
| A46b | 21 | 2570 |
| A46c | 135 | 3340 |
| A46d | 9 | 11400 |
| A46e | 6160 | 26000 |
| A46f | 2140 | 59600 |
| A47 | 9.3 | 4200 |
| A48 | 73 | 75 |
| A49 | 200 | 130 |
| A50 | 720 | 790 |
| A51 | 7.8 | 347 |
| A52 | 72 | 31 |
| A53 | 7.8 | 1200 |
| A54 | 60 | 580 |
| A55 | 9 | 22 |
| A56 | 27 | 560 |
| A57 | 50 | 114 |
| A58 | 2.8 | 7.3 |
| A59 | 28 | 1880 |
| A60 | 17 | 125 |
| A61 | 8.6 | 177 |
| A62 | 48 | 40 |
| A63 | 43 | 83 |
| A64 | 230 | 2800 |
| A65 | 12 | 1230 |
| A66 | 409 | 9840 |
| A67 | 130 | 10 |
| A68 | 3600 | 140 |
| A69a | 25 | 130 |
| A69b | 4900 | 3000 |
| A70 | 640 | 38 |
| A71a | 2100 | 42000 |
| A71b | 130 | 40000 |
| A72a | 850 | 1200 |
| A72b | 15 | 83 |
| A73 | 630 | 630 |
| A74 | 830 | 3.8 |
| A74a | 224 | 2.2 |
| A74b | 5450 | 187 |
| A75 | 200 | 42 |
| A76 | 290 | 200 |
| A77 | 600 | 9.2 |
| A78 | 640 | 2410 |
| A79 | 115 | 2480 |
| A80 | 663 | >10000 |
| A81 | 60 | 461 |
| A82 | 90 | 1020 |
| A83 | 170 | 81 |
| A84 | 128 | 2890 |
| A85 | 1180 | 5280 |
| A86 | 1940 | 22 |
| A87 | 211 | 114 |
| A88 | 9440 | 118 |
| A89 | 296 | 4.3 |
| A90 | 1900 | 13 |
| A91 | 533 | >10000 |
| A92a | >10000 | >10000 |
| A92b | 2840 | >10000 |
| A93 | 3660 | >10000 |
| A94 | 98 | 9130 |
| A95 | 53 | 12200 |
| A96 | 255 | >10000 |
| A97 | 1 | 999 |
| A97a | 8.6 | 3380 |
| A97b | 1610 | 5120 |
| A98 | 410 | 27000 |
| A98a | 15 | 1060 |
| A98b | 1760 | >10000 |
| A99 | 2790 | >10000 |
| A100 | 200 | 3780 |
| A101 | 707 | >10000 |
| A102 | 288 | >10000 |
| A103 | 107 | 208 |
| A103a | 241 | 403 |
| A103b | 2690 | 809 |
| A104 | >10000 | >10000 |
| A105 | 258 | 8920 |
| A106a | 2190 | >10000 |
| A106b | 183 | >10000 |
| A107 | 272 | >10000 |
| A108 | >10000 | 3830 |
| A109 | 7850 | 6170 |
| A110a | 151 | 1160 |
| A110b | 1230 | 9030 |
| A111 | 96 | >10000 |
| A112 | 220 | >10000 |
| A113 | 1470 | >10000 |
| A114 | 6600 | >10000 |
| A115a | >10000 | >10000 |
| A115b | 378 | 105000 |
| A116 | 146 | 5760 |
| A117 | 3870 | >10000 |
| A118 | 185 | >150000 |
| A119a | 15 | 8090 |
| A119b | 964 | 4560 |
| A120a | 2930 | 176 |
| A120b | >10000 | 2260 |
| A121a | 3020 | 1300 |
| A121b | >10000 | 4160 |
| A122 | 635 | 10.5 |
| A123 | 1740 | 6030 |
| A124 | >10000 | >10000 |
| A125a | >10000 | >10000 |
| A125b | 5270 | >10000 |
| A126 | 7.2 | 449 |
| A127 | 260 | 56 |
| A127a | 84 | 37 |
| A127b | 3710 | 177 |
| A128 | 112 | 31 |
| A129 | 33 | 2350 |
| A130 | 9080 | 15 |
| A131 | 8990 | 287 |
| A132a | 5.9 | 8.9 |
| A132b | 1780 | 330 |
| A133 | 9390 | >10000 |
| A134 | 35 | 1940 |
| A135a | 97 | 398 |
| A135b | 1300 | 428 |
| A136 | 317 | 61900 |
| A137 | 106 | >10000 |
| A138 | 480 | 1280 |
| A139a | 112 | >10000 |
| A139b | 5740 | >10000 |
| A140 | 189 | 82 |
| A141 | 696 | 6630 |
| A142 | 1220 | 6580 |
| A143 | 253 | >10000 |
| A144 | 42 | 105000 |
| A145 | 55 | 20300 |
| A146a | 283 | 440 |
| A146b | 4690 | >10000 |
| A148 | 190 | 34 |
| A149a | 4160 | 4120 |
| A149b | 2250 | 191 |
| A150a | 2750 | 5830 |
| A150b | 543 | 82.5 |
| A151 | 2040 | >10000 |
| A152 | 1770 | 4380 |
| A153a | 9340 | >10000 |
| A153b | 545 | >10000 |
| A154 | 104 | 9210 |
| A155a | 1380 | 728 |
| A155b | 7.6 | 32 |
| A156 | 18 | 1750 |
| A157 | 1030 | 2990 |
| A158 | 381 | >10000 |
| A159 | 30 | 190 |
| A160 | 247 | 52 |
| A161a | >10000 | >10000 |
| A161b | 93 | 78200 |
| A162a | 4480 | >10000 |
| A162b | 1050 | >10000 |
| A163 | 4420 | >10000 |
| A164 | 72 | 8970 |
| A165 | 2840 | >10000 |
| A166 | 2380 | >10000 |
| A167 | 1020 | >10000 |
| A168 | 28 | 6080 |
| A169 | 701 | >10000 |
| A170 | 887 | >10000 |
| A171a | 65 | 8340 |
| A171b | 1360 | >10000 |
| A172a | 4320 | >10000 |
| A172b | 14 | 20400 |
| A173a | 502 | >10000 |
| A173b | >10000 | >10000 |
| A174 | 57900 | 105000 |
| A175 | 305 | >10000 |
| A176 | 93 | 5670 |
| A177a | 70 | 8210 |
| A177b | 3600 | >10000 |
| A178 | 6900 | >10000 |
| A179a | 7880 | >10000 |
| A179b | >10000 | >10000 |
| A180a | 15 | 730 |
| A180b | 1860 | >10000 |
| A181 | >10000 | >10000 |
| A182 | 230 | >10000 |
| A183a | 110 | 7740 |
| A183b | 2080 | >10000 |
| A184 | 6010 | >10000 |
| A185a | >10000 | >10000 |
| A185b | 34 | 3990 |
| A186 | 59 | 6370 |
| A187a | 5920 | 5740 |
| A187b | 199 | 6320 |
| A188 | 786 | >10000 |
| A189 | 3100 | >10000 |
| A190a | 172 | >10000 |
| A190b | >10000 | >10000 |
| A191a | 6780 | >10000 |
| A191b | 1180 | >10000 |
| A192 | 59 | >10000 |
| A193 | 5730 | >10000 |
| A194a | 155 | 6350 |
| A194b | 1190 | 5090 |
| A195 | 1900 | 1190 |
| A196a | 1350 | >10000 |
| A196b | 38 | 93200 |
| A197a | 2050 | >10000 |
| A197b | 169 | >10000 |
| A198a | >10000 | >10000 |
| A198b | >10000 | >10000 |
| A199a | 2460 | >10000 |
| A199b | 803 | >10000 |
| A200a | 102 | 7080 |
| A200b | 5380 | >10000 |
| A201a | 43 | >10000 |
| A201b | >10000 | >10000 |
| A202a | 176 | 700 |
| A202b | 3030 | 1280 |
| A203a | 2210 | >10000 |
| A203b | 233 | >10000 |
| A204a | 3290 | >10000 |
| A204b | 298 | >10,000 |
| A205 | 151 | 63700 |
| A206 | 2270 | 2590 |
| A207 | 4050 | >10000 |
| A208 | >10000 | >10000 |
| A209 | 50 | 21600 |
| A210 | 369 | 11200 |
| A211 | 234 | 2760 |
| A212 | 1130 | >10000 |
| A213a | 1360 | >10000 |
| A213b | >10000 | >10000 |
| A214 | 3430 | >10000 |
| A215a | >10000 | >10000 |
| A215b | 1140 | >10000 |
| A216 | 2370 | >10000 |
| A217 | 378 | >10000 |
| A218a | 166 | >150000 |
| A218b | 3960 | >10000 |
| A219 | >10000 | >10000 |
| A220 | 67 | 8690 |
| A221a | 216 | 1090 |
| A221b | >10000 | 2370 |
| A222a | 452 | 5250 |
| A222b | >10000 | 7420 |
| A223 | >10000 | >10000 |
| A224a | 122 | 1090 |
| A224b | 4940 | 4150 |
| A225 | 99 | 7450 |
| A226 | 2290 | 8430 |
| A227a | >10000 | >10000 |
| A227b | 691 | >10000 |
| A228 | 9200 | >10000 |
| A229 | 166 | 24100 |
| A230a | 1440 | >10000 |
| A230b | 494 | 6200 |
| A231a | >10000 | 1120 |
| A231b | >10000 | 303 |
| A232 | 3110 | 8490 |
| A233 | 33 | 6980 |
| A234 | 159 | 7330 |
| A235 | 5380 | >10000 |
| A236a | >10000 | >10000 |
| A236b | 178 | 77400 |
| A237 | 1350 | >10000 |
| A238a | 2390 | >10000 |
| A238b | 256 | 1240 |
| A239 | 391 | >10000 |
| A240 | 2010 | 948 |
| A241 | 270 | 6430 |
| A242 | >10,000 | >10000 |
| A243 | 1350 | >10000 |
| A244 | >10000 | >10000 |
| A245 | 87 | >10000 |
| A246 | 1070 | 9720 |
| A247 | 283 | >10000 |
| A248 | 579 | >10000 |
| A249 | 1970 | 5790 |
| A250 | 3010 | >10000 |
| A251 | 3270 | >10000 |
| A252 | 1370 | >10000 |
| A253 | 8980 | 1950 |
| A254 | 307 | >10000 |
| A255 | 194 | >10000 |
| A256 | 315 | >10000 |
| A257 | 652 | >10000 |
| A258 | >10000 | >10000 |
| A259 | 380 | >10000 |
| A260 | 394 | >10000 |
| A261 | 2350 | >10000 |
| A262 | 150 | >10000 |
| A263 | 85 | 41800 |
| A264 | 215 | >10000 |
| A265 | 736 | >10000 |
| A266 | 298 | >10000 |
| A267a | >10000 | >10000 |
| A267b | 70 | >150000 |
| A268 | 133 | 16300 |
| A269 (RII) | 35 | >10000 |
| A269a | 365 | >10000 |
| A269b | 6850 | >10000 |
| A269c | >10000 | >10000 |
| A269d | 21 | 51700 |
| A270 | 169 | 40100 |
| A271 | >10000 | >10000 |
| A272 | 411 | >10000 |
| A273 | 5550 | >10000 |
| A274 | >10000 | >10000 |
| A275a | 7110 | >10000 |
| A275b | 4390 | >10000 |
| A276 | 131 | >150000 |
| A277 | 2110 | >10000 |
| A278 | 64 | 55100 |
| A279 | >10000 | >10000 |
| A280 | 34 | 26000 |
| A281a | 77 | 6650 |
| A281b | 1120 | 11200 |
| A282a | 5350 | 182 |
| A282b | 6960 | 10500 |
| A283 | 881 | 12400 |
| A284 | >10000 | 6480 |
| A285 | >10000 | >10000 |
| A286a | 2610 | >10000 |
| A286b | 17 | 51500 |
| A287a | 9680 | >10000 |
| A287b | 567 | >10000 |
| A288 | 263 | 3400 |
| A289 | 1130 | >10000 |
| A290 | 425 | 56400 |
| A291 | 5450 | 115000 |
| A292 | 1910 | >150000 |
| A293 | 237 | 9950 |
| A294 | 3860 | 69600 |
| A295 | 563 | 5690 |
| A296 | 189 | 3900 |
| A297 | >10000 | 8180 |
| A298 | 8910 | >150000 |
| A299 | 90 | 66100 |
| A300 | 524 | 54000 |
| A301 | 131 | >150000 |
| A302 | 68 | 35400 |
| A303 | 19 | 102000 |
| A304 | 763 | 3660 |
| A305 | 1190 | 1490 |
| A306 | 495 | 18200 |
| A307 | 1750 | 2470 |
| A308 | 72 | 88300 |
| A309 | 880 | 108000 |
| A309a | >10000 | 118000 |
| A309b | 369 | 87000 |
| A310 | 127 | 94700 |
| A311 | 41 | 6630 |
| A312 | 238 | 66000 |
| A313 | 41 | 48000 |
| A313a | 8290 | 116000 |
| A313b | 51 | 61600 |
| A314 | 689 | 84300 |
| A315 | 3590 | 61000 |
| A316 | >10000 | 67000 |
| A317 | 21 | 8650 |
| A318 | 15 | 6490 |
| A319 | 23 | 49800 |
| A320 | 159 | 11600 |
| A321 | 4020 | >150000 |
| A322 | 59 | 189 |
| A323 | 7 | 10700 |
| A324 | 4020 | >150000 |
| A325 | 89 | 103000 |
| A326 | 1990 | 105000 |
| A327 | 19 | >150000 |
| A328 | 143 | 83000 |
| A329 | 91 | >150000 |
| A330 | 606 | 7360 |
| A331 | 46 | 31800 |
| A332 | 169 | 123 |
| A333 | 210 | 10600 |
| A334a | 978 | 308 |
| A334b | >10000 | 893 |
| A335 | 142 | 610 |
| A336 | 60 | 1570 |
| A337 | 283 | 203 |
| A338 | 160 | 527 |
| A339 | 361 | 5360 |
| A340 | 248 | 485 |
| A341 | 4250 | 1620 |
| A342 | 353 | 229 |
| A343 | 675 | 246 |
| A344 | 198 | 108 |
| A345 | 205 | 852 |
| A346 | 556 | 29 |
| A347a | 4,800 | 173 |
| A347b | 223 | 8 |
| A348a | 3,880 | 177 |
| A348b | 138 | 9 |
| A349a | 462 | 490 |
| A349b | 4240 | 1580 |
| A350 | 249 | 945 |
| A351a | 167 | 274 |
| A351b | 3250 | 1190 |
| A352 | 6 | 5400 |
| A353a | 190 | 32 |
| A353b | 2490 | 797 |
| A354a | 3130 | 996 |
| A354b | 260 | 130 |
| A355a | 6420 | 10900 |
| A355b | 176 | 2150 |
| A356 | 1570 | 635 |
| A357a | 2540 | 2450 |
| A357b | 12 | 646 |
| A358 | 373 | 5830 |
| A359 | 299 | 117 |
| A360a | 3800 | 142 |
| A360b | 134 | 2.7 |
| A361 | 554 | 5.1 |
| A362a | 2510 | 922 |
| A362b | 300 | 33 |
| A363 | 55 | 166 |
| A364 | 288 | 2520 |
| A365 | 4710 | 794 |
| A366 | 644 | 366 |
| A367 | 3590 | 19300 |
| A368a | 6650 | 4900 |
| A368b | 382 | 91 |
| A369 | 1090 | 294 |
| A370 | >10000 | 363 |
| A371 | >10000 | 717 |
| A372 | 3450 | 127 |
| A373 | 3220 | 21800 |
| A374 | 51 | 618 |
| A375 | 944 | 200 |
| A376 | 602 | 206 |
| A377 | 173 | 94 |
| A378 | 774 | 140 |
| A379 | 571 | 294 |
| A380 | 667 | 161 |
| A381a | 290 | 459 |
| A381b | 2330 | 2090 |
| A382 | 499 | 79 |
| A383a | 158 | 245 |
| A383b | 4590 | 2790 |
| A384 | 99 | 1750 |
| A385 | 498 | 481 |
| A386 | 418 | 689 |
| A387 | 4890 | 75000 |
| A388 | 1250 | 1390 |
| A389 | 201 | 43 |
| A390 | 490 | 4580 |
| A391 | 1650 | 1180 |
| A392a | 1480 | 1320 |
| A392b | >10000 | 1070 |
| A393 | 795 | 108 |
| A394a | 5400 | 454 |
| A394b | 125 | 30 |
| A395 | 272 | 222 |

### 2) Cell Culture and DGKα/ζ Knockout Jurkat Cell Line Construction

Jurkat cells and human PBMC were maintained in RPMI 1640 medium (Gibco) supplemented with 10% fetal bovine serum (FBS, Thermo Scientific), 100 units/mL penicillin and 0.1 mg/mL streptomycin (Gibco) in a humidified 37° C. environment with 5% CO₂. HepG2-OS8 cells, which express the single chain variable fragment (scFv) of an anti-human CD3 mAb OKT3 fused to the C-terminal domain (113-220) of mouse CD8a which includes hinge, transmembrane and cytoplasmic domains, were maintained in MEM medium (Gibco) supplemented with 10% fetal bovine serum (FBS, Thermo Scientific), 100 units/mL penicillin and 0.1 mg/mL streptomycin (Gibco) in a humidified 37° C. environment with 5% CO₂.

Jurkat cells were infected with the lentivirus expressing spCas9 and sgRNA targeting human DGKα or DGKζ. Cell clones that stably knockout with DGKα/ζ were established and maintained in the RPMI 1640 complete medium. Knockout efficiency of eSPCas9-Lenticrispr DGKα or DGK sgRNA in single cell clone was determined using genomic sequencing and immunoblotting method. Selected compounds did not induce DGKζ a or DGK independent IL-2 production in DGKα/ζ KO jurkat cells.

### 3) Non-stimulated Phosphorylated ERK Detection Assay

Cellular non-stimulated phospho-ERK were measured using a AlphaLISA-based method (Beaudet, Lucille, et al. Nature Methods. 2008, 5.12: an8-an9). Jurkat cells were subcultured in T75 flasks. The next day, growth medium was replaced to serum free RPMI 1640 for 4 hours or overnight. The cells were then seeded into 96-well plates and treated with compounds. After 2 h compound treatment, lysis buffer (PerkinElmer) was added to each well. Plates were then incubated at room temperature with shaking for 30 minutes. A total of 10 μL of cell lysate from each well of a 96-well plate was transferred to a 384-well white assay plate. Phosphor-ERK was quantitated using the AlphaLISA kit (Cat #ALSU-PERK-A10K) as described by the manufacturer manual (PerkinElmer). AlphaLISA signals were measured using a PHERAstar FSX reader (BMG Labtech). Selected compounds did not elevate ERK phosphorylation in Jurkat cells without TCR activation.

### 4) IL-2 Production Assay in Human PBMC

Frozen human PBMC were thawed in RPMI 1640 medium and incubated at 37° C. overnight. OS8 overexpressing HepG2 cells were seeded into 384-well plates overnight. The next day, PBMC were added into the 384-well plates and then treated with compounds. PBMC and HepG2-OS8 cells were co-cultured for 48 hours at 37° C. Culture supernatant was collected for subsequent measurement of IL-2 concentration by a TR-FRET-based method (Degorce, Frangois, et al. Current chemical genomics. 2009, 3: 22) as described by the manufacturer manual (Cisbio). FRET signals were measured using a PHERAstar FSX reader (BMG Labtech). Selected compounds showed good potency in Human PBMC assay.

### 5) Explorative Acute Toxicity Study in BALB/c Mice

The test article was dissolved in vehicle formulation (DMA: 30% solutol HS-15(w/v): saline=20:20:60) and injected through tail vein into BALB/c mice at the doses of 2 and/or 10 mg/kg. Continuous clinical observation within 2 hours post injection was performed. Selected compounds were well tolerant at the doses of 2 and/or 10 mg/kg.

It is to be understood that, if any prior art publication is referred to herein, such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

What is claimed is:

1. A compound of formula (I), 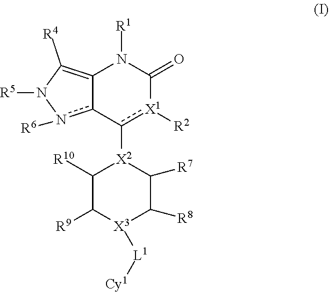
or a stereoisomer or pharmaceutically acceptable salt thereof
wherein
X¹ is C or N,
each of X² and X³ is independently selected form —N— or —CH—;
the symbol [symbol] is a single or double bond,
R¹ is hydrogen, or alkyl optionally substituted with deuterium, halogen, hydroxy, alkoxy or cycloalkyl;
R² is hydrogen, halogen, alkyl or cyano, provided that R² is absent when X¹ is N and the bond [symbol] attached to X¹ is a double bond;
R⁴ is hydrogen, halogen, or alkyl, wherein the alkyl is optionally substituted with halogen or —OR^{4a}, wherein R^{4a} is hydrogen, alkyl, alkenyl, alkynyl, cycloalkyl or heterocycloalkyl, wherein alkyl, alkenyl, alkynyl, cycloalkyl or heterocycloalkyl is optionally substituted with —C₁₋₆alkyl, —C₁₋₆ alkoxy or —C₃₋₈cycloalkyl;
R⁵ is hydrogen, alkyl, alkenyl, alkynyl, cycloalkyl, R^{5a}—C(O)—, R^{5a}—C(O)O—, R^{5a}—O—C(O)—, R^{5a}—C(O)NR^{5b}—, R^{5a}—NR^{5b}—C(O)—, R^{5a}—SO₂— or heterocyclyl, wherein said alkyl or alkenyl is unsubstituted or substituted with halogen, cyano, —C(O)OR^{5c}, —C(O)R^{5c}, —C(O)NR^{5c}R^{5d}, heterocyclyl, alkoxy, hydroxy, cycloalkyl, or NR^{5c}R^{5d}; and wherein each of said cycloalkyl and heterocyclyl is unsubstituted or substituted with alkyl, cyano or halogen substituted alkyl, cyano, —C(O)OR^{5c}, —C(O)R^{5c}, —C(O)NR^{5c}R^{5d}, heterocyclyl, alkoxy, hydroxy, cycloalkyl, NR^{5c}R^{5d}, or R^{5c}—SO₂—, wherein R^{5a} and R^{5b} are each independently hydrogen, alkyl, or cycloalkyl; and wherein R^{5c} and R^{5d} are hydrogen or alkyl;
R⁶ is hydrogen, halogen, alkyl which is unsubstituted or substituted with halogen or cyano, provided that R⁶ is absent when the bond [symbol] attached to the nitrogen to which R⁶ is attached is a double bond;
each of R⁷, R⁹, R⁸, and R¹⁰ is independently hydrogen, alkyl, alkoxy, or —C(O)R^{7a}, wherein said alkyl is unsubstituted or substituted with halogen, cyano, hydroxy, alkoxy or —C(O)R^{7a}, and wherein R^{7a} is hydrogen, alkyl, or alkoxy, provided that at least one of R⁷ and R⁹ is not hydrogen;
or R⁷ and R⁹ are each hydrogen and R⁸ and R¹⁰ together form a bridge containing at least one —CH₂— moiety in addition to the two bridgehead atoms; or R⁸ and R¹⁰ are each hydrogen and R⁷ and R⁹ together form a bridge containing at least one —CH₂— moiety in addition to the two bridgehead atoms;
L¹ is a direct bond, —O—, —N(R^{L})—, -alkylene- or —C(O)—, wherein said -alkylene- is unsubstituted or substituted with —C(O)NR^{L1}, and R^{L} is hydrogen or alkyl, wherein R^{L1} is hydrogen, alkyl, or alkoxyalkyl;
Cy¹ is aryl, heterocyclyl, heteroaryl, or cycloalkyl, each of which is unsubstituted or substituted with one, two or three substituents R^{3a}, wherein R^{3a} is selected from deuterium, hydroxy, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, cycloalkyl, heterocyclyl or heterocyclyloxy, wherein said alkyl moiety in the group alkyl or alkoxy is unsubstituted or substituted with deuterium, halogen, alkoxy, hydroxy, cyano or heterocyclyl; said cycloalkyl or heterocyclyl is unsubstituted or substituted with alkoxy, alkyl, halogen, or hydroxy, wherein R^{3b} and R^{3c} are each independently hydrogen or alkyl.

2. The compound according to claim 1, wherein the compound of formula (I) is 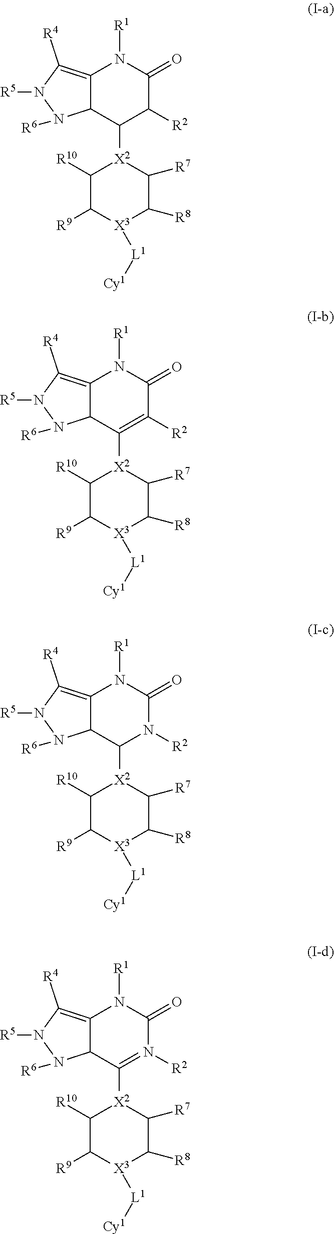 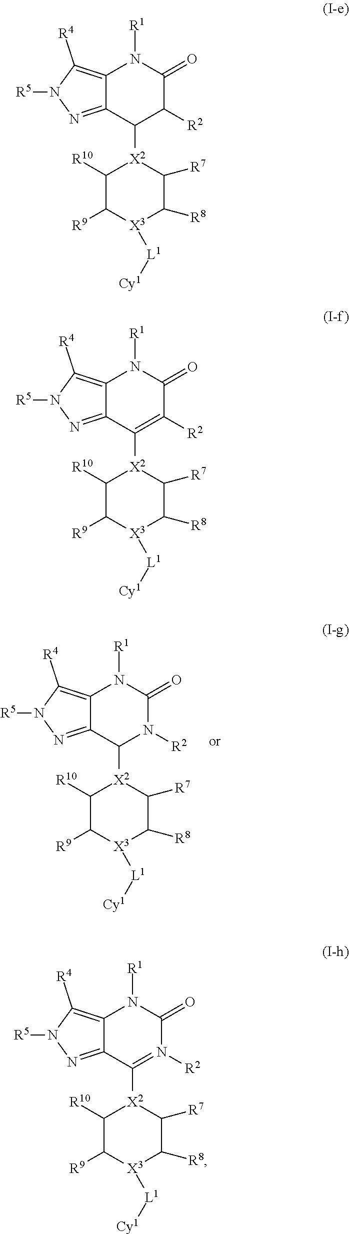
or a stereoisomer or pharmaceutically acceptable salt thereof.

3. (canceled)

4. The compound according to claim 1, wherein R¹ is hydrogen, methyl, methyl-d3, ethyl, isopropyl, 2-hydroxyethyl, 2-methoxyethyl, 2,2,2-trifluoroethyl, 2,2-difluoroethyl, or cyclopropylmethyl; preferably R¹ is hydrogen, methyl, ethyl or methyl-d3; more preferably R¹ is methyl or methyl-d3.

5. The compound according to claim 1, wherein R² is hydrogen, halogen, C₁₋₄alkyl or cyano; preferably R² is hydrogen, F, Br, Cl or CN.

6. (canceled)

7. The compound according to claim 1, wherein R⁴ is hydrogen, fluoro, chloro, bromo, methyl, trifluoromethyl, ethyl, or 2,2,2-trifluoroethyl; preferably R⁴ is hydrogen.

8. (canceled)

9. (canceled)

10. The compound according to claim 1, wherein R⁵ is hydrogen, CN—CH₂—, —CH₂C(O)—OMe, —CH(CH₃)CN, oxiran-2-ylmethyl-, prop-2-yn-1-yl, but-2-yn-1-yl, prop-1-en-2-yl, methyl, isopropyl, —CH₂CH₂—OMe, —CH₂C(O)NH₂, —CH₂CH₂—OH, cyclopropyl-CH₂—, —CH₂CH₂N(CH₃)₂, CH₃—SO₂—, cyclopropyl, cyclobutyl, cyclopropyl-C(O)—, 1-cyanocyclopropyl, 2-cyanocyclopropyl, 2-cyanocyclobutyl, 3-(cyanomethyl)-1-(ethylsulfonyl)azetidine-3-yl, or 1-cyano-2-cyclopentyleth-2-yl; preferably, R⁵ is hydrogen, CN—CH₂—, —CH₂C(O)—OMe, —CH(CH₃)CN, oxiran-2-ylmethyl-, prop-2-yn-1-yl, but-2-yn-1-yl, or prop-1-en-2-yl; more preferably, R⁵ is CN—CH₂—, prop-2-yn-1-yl, but-2-yn-1-yl, or prop-1-en-2-yl; more preferably R⁵ is CN—CH₂—, —CH(CH₃)CN, prop-2-yn-1-yl, but-2-yn-1-yl, or prop-1-en-2-yl.

11. The compound according to claim 1, wherein R⁶ is absent.

12. (canceled)

13. The compound according to claim 1, wherein R⁷ and R⁹ are each independently hydrogen, methyl, ethyl, methoxymethyl, 2-methoxyethyl, cyanomethyl, hydroxyethyl, hydroxymethyl, methoxycarbonyl, difluoromethyl, provided that at least one of R⁷ and R⁹ is not hydrogen.

14. The compound according to claim 1, wherein R⁷ is methyl, and R⁹ is methyl; or R⁷ is ethyl, and R⁹ is ethyl; or R⁷ is methyl, and R⁹ is ethyl; or R⁷ is methyl, and R⁹ is methoxycarbonyl; or R⁷ is hydrogen, and R⁹ is methyl; or R⁷ is hydrogen, and R⁹ is ethyl.

15. (canceled)

16. The compound according to claim 1, wherein R⁸ and R¹⁰ are each hydrogen.

17. The compound according to claim 1, wherein L¹ is a direct bond, —O—, —N(R^{L})—, -alkylene- or —C(O)—, wherein R^{L} is hydrogen or alkyl; preferably L¹ is C₁₋₄alkylene, preferably C₁₋₂alkylene; more preferably L¹ is a direct bond, —CH₂—, —CH(CH₃)—, —CH(CH₂CH₃)—, —CH(CHF₂)—, —N(H)—, —N(CH₃)—, —O—, —CH(C(O)—NHCH₂CH₂OCH₃)— or —C(CH₃)₂—;
much more preferably L¹ is —CH₂— or CH(CH₃)—.

18. The compound according to claim 1, wherein X² is N, and X³ is N; or X² is N, and X³ is CH; or X² is CH, and X³ is N; or X² is CH, and X³ is N; preferably X² is N, and X³ is N.

19. (canceled)

20. (canceled)

21. The compound according to claim 1, wherein Cy¹ is phenyl, which is unsubstituted or substituted with one R^{3a} at position 4 and optionally substituted with R^{3a} on another position.

22. The compound according to claim 1, wherein Cy¹ is 2,3-dihydrobenzo[b][1,4]dioxin-6-yl, 3,3-dimethyl-2,3-dihydrobenzo[b][1,4]dioxin-6-yl, 2,2-difluorobenzo[d][1,3]dioxol-4-yl, 3,4-dihydro-2H-benzo[b][1,4]oxazin-6-yl, or

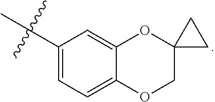 [SMILES: *c1ccc2c(c1)OC1(CC1)CO2]

23. The compound according to claim 1, wherein Cy¹ is a monocyclic 5- to 9-membered heterocyclyl or a bicyclic 7- to 10-membered heterocyclyl which is unsubstituted or substituted with one, two or three R^{3a}.

24. (canceled)

25. The compound according to claim 1, wherein Cy¹ is a monocyclic 5- to 9-membered heteroaryl or a bicyclic 7- to 10-membered heteroaryl which is unsubstituted or substituted with one, two or three R^{3a}.

26-29. (canceled)

30. The compound according to claim 25, wherein Cy¹ is quinoxalinyl, e.g., quinoxalin-2-yl, quinoxalin-3-yl, quinoxalin-4-yl, quinoxalin-5-yl, quinoxalin-6-yl, quinoxalin-7-yl, quinoxalin-8-yl, preferably quinoxalin-6-yl, which is which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with deuterium, halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R³⁴)—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, methyl-d3, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, 1-hydroxyazetidin-3-yl, (2,2-dimethylmorpholino)methyl, 4-((2S,6R)-2,6-dimethylmorpholino)methyl, or (4,4-difluoropiperidin-1-yl)methyl.

31. The compound according to claim 25, wherein Cy¹ is quinoxalin-6-yl, which is unsubstituted or substituted with one, two or three R^{3a}, wherein R^{3a} is deuterium, methyl, ethyl, trifluoromethyl, methoxy, isopropoxy, difluoromethoxy, fluoro, chloro, cyano, amino, or cyclopropyl.

32-34. (canceled)

35. A pharmaceutical composition comprising a compound according to claim 1, or a stereoisomer or pharmaceutically acceptable salt thereof, and a pharmaceutically acceptable excipient.

36. A method of treating a disease, comprising administering to a patient in need of treatment a therapeutically effective amount of a compound according to claim 1, or a stereoisomer or pharmaceutically acceptable salt thereof, wherein the disease is cancer.


SEQUENCE LISTING (2 sequences)

SEQ ID NO: 1 - DNA, 2841 bp, synthetic construct
  misc_feature 1..2841: note = His-TEV-hDGK-(M1-V929)
  source 1..2841: mol_type = other DNA
atgcatcaccatcaccatcaccatcacgagaacctgtacttccagggatccatggaaccccgtgatggtagccccgaagctcgtagctccgattccgagtccgccagcgcttcctcctccggtagcgaacgtgacgctggtcccgagcccgacaaagctccccgtcgtctgaataagcgccgttttcccggtctccgtctgttcggccaccgcaaggccatcactaagtccggtctccagcatctggctcctcctcctcctacccccggtgctccttgctccgaatccgagcgccagattcgctccactgtggattggtccgaaagcgccacctatggtgagcatatctggttcgagaccaacgtctccggcgacttctgttatgtcggtgagcaatactgtgtggctcgtatgctgcagaagtccgtgtcccgccgtaaatgcgccgcttgcaaaatcgtggtccataccccttgcatcgagcaactggagaaaatcaacttccgctgcaagcccagctttcgtgagtccggttcccgcaacgtgcgcgaacctactttcgtgcgccaccactgggtgcatcgtcgtcgccaagacggcaaatgccgccactgcggcaaaggttttcagcagaaattcaccttccacagcaaggagatcgtcgccatcagctgcagctggtgcaaacaagcttaccattccaaagtgagctgcttcatgctccagcagatcgaagagccttgctctctgggtgtgcatgctgctgtcgtgattccccctacttggattctgcgtgctcgccgtccccagaacactctgaaggcctccaaaaagaagaagcgcgccagcttcaagcgtaagagctccaaaaagggtcccgaagagggccgttggcgtcccttcatcatccgccctactccttcccctctgatgaagcctctgctggtcttcgtcaaccctaagagcggcggcaaccaaggtgctaaaatcatccagtccttcctctggtatctgaaccctcgtcaagtgttcgacctcagccaaggcggtcctaaggaggctctggagatgtaccgcaaggtccacaatctgcgcatcctcgcttgtggtggcgatggcaccgtgggctggattctgtccactctggaccaactgcgtctgaaacctcccccccccgtggctattctgcctctcggtaccggcaacgatctggctcgtactctgaattggggtggtggctacaccgatgagcccgtgtccaagattctgtcccacgtcgaagaaggcaatgtcgtccaactggaccgttgggacctccacgccgaacccaaccccgaggctggccccgaggaccgtgacgagggcgctactgaccgtctgcccctcgacgtcttcaataattacttctctctgggctttgacgctcacgtgactctggaatttcatgaaagccgcgaggccaaccccgagaagttcaattcccgtttccgcaacaagatgttctacgctggcaccgccttcagcgacttcctcatgggctccagcaaggacctcgctaagcatatccgcgtggtgtgcgatggcatggatctgacccctaagatccaagatctgaagccccaatgtgtcgtgtttctgaacatcccccgctactgcgctggtactatgccttggggccatcccggtgaacaccatgacttcgaacctcagcgtcatgatgacggctatctggaggtgatcggtttcaccatgacctccctcgctgctctgcaagtgggtggccacggcgaacgtctgactcaatgccgcgaggtggtgctgaccaccagcaaagccatccccgtccaagtggatggtgagccttgcaagctggccgcctcccgtatccgtatcgctctccgcaatcaagctaccatggtccagaaggccaaacgccgcagcgctgctcctctccacagcgaccaacaacccgtccccgaacagctgcgcatccaagtgtcccgtgtcagcatgcatgactacgaggctctgcactacgacaaggaacagctgaaggaagccagcgtgcctctgggtactgtggtcgtgcccggtgacagcgatctggagctctgccgtgcccacatcgagcgtctgcagcaagagcccgacggtgctggtgccaagagccctacttgccaaaaactctcccccaagtggtgtttcctcgacgctaccaccgccagccgcttctaccgcattgatcgcgcccaagagcatctgaactatgtcaccgagatcgctcaagacgagatctacatcctcgaccccgaactcctcggtgctagcgcccgtcccgacctccccactcctacctcccctctgcccacttccccttgttcccccacccctcgtagcctccaaggtgatgctgcccctcctcaaggtgaggagctcattgaggccgctaagcgtaacgatttctgcaagctccaagagctgcatcgtgctggtggcgacctcatgcaccgcgatgagcagagccgcactctgctgcaccacgctgtgtccactggtagcaaggacgtggtgcgctatctgctggaccacgctcctcccgagatcctcgacgctgtggaagaaaacggcgagacttgcctccaccaagctgctgctctgggtcaacgtaccatctgccactacatcgtcgaagctggtgcttctctgatgaagaccgaccagcaaggtgatactccccgtcagcgcgccgagaaagcccaagacaccgaactggctgcctatctggagaaccgtcagcactaccagatgattcagcgtgaagaccaagagaccgccgtgtaa

SEQ ID NO: 2 - protein, 929 aa, synthetic construct
  REGION 1..929: note = His-TEV-hDGK-(M1-V929)
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAV